(19) United States
(12) Patent Application Publication
(10) Pub. No.: US 2025/0282857 A1
(43) Pub. Date: Sep. 11, 2025

(54) TGF-BETA INHIBITORS AND USE THEREOF

(71) Applicant: SCHOLAR ROCK, INC., Cambridge, MA (US)

(72) Inventors: Si Tuen LEE-HOEFLICH, Cambridge, MA (US); Christopher BRUECKNER, Cambridge, MA (US); Constance MARTIN, Cambridge, MA (US); Ryan FAUCETTE, Cambridge, MA (US)

(21) Appl. No.: 18/283,904

(22) PCT Filed: Mar. 25, 2022
(86) PCT No.: PCT/US2022/022063

§ 371 Date: Sep. 25, 2023

Related U.S. Application Data
(60) Provisional application No. 63/166,824, filed on Mar. 26, 2021.
(60) Provisional application No. 63/202,260, filed on Jun. 3, 2021.
(60) Provisional application No. 63/302,999, filed on Jan. 25, 2022.
(60) Provisional application No. 63/313,386, filed on Feb. 24, 2022.

(51) Int. Cl. C07K 16/22; A61K 39/395; A61K 45/06; A61P 35/00; G01N 33/574

(52) U.S. Cl. CPC C07K 16/22; A61K 39/3955; A61K 45/06; A61P 35/00; G01N 33/57488; G01N 2333/495; G01N 2333/522

(57) ABSTRACT

The present disclosure provides TGFβ inhibitor therapy for treating immunosuppressive conditions, such as cancer. Methods of predicting and monitoring therapeutic response are disclosed. Related compositions, methods and therapeutic use are also disclosed.

### RELATED APPLICATIONS

This application is a U.S. national stage application under 35 U.S.C. § 371 of International Application No. PCT/US2022/022063, filed on Mar. 25, 2022, which claims the benefit of and priority to U.S. Provisional Applications 63/166,824 filed Mar. 26, 2021; 63/202,260 filed Jun. 3, 2021; 63/302,999 filed Jan. 25, 2022; and 63/313,386 filed Feb. 24, 2022, each entitled “TGF-BETA INHIBITORS AND USE THEREOF.” The contents of all aforementioned applications are expressly incorporated herein by reference in their entirety.

### SEQUENCE LISTING

The ASCII-format Sequence Listing named “15094-0013-00304_SL.txt,” which was created on Sep. 22, 2023 with a size of 251,422 bytes, is filed herewith and incorporated by reference in its entirety.

### FIELD

The instant application relates generally to TGFβ inhibitors and therapeutic use thereof, as well as related assays for diagnosing, monitoring, prognosticating, and treating disorders, including cancer.

### BACKGROUND

Transforming growth factor beta 1 (TGFβ1) is a member of the TGFβ superfamily of growth factors, along with two other structurally related isoforms, namely, TGF32 and TGF33, each of which is encoded by a separate gene. These TGFβ isoforms function as pleiotropic cytokines that regulate cell proliferation, differentiation, immunomodulation (e.g., adaptive immune response), and other diverse biological processes both in homeostasis and in disease contexts. The three TGFβ isoforms signal through the same cell-surface receptors and trigger similar canonical downstream signal transduction events that include the SMAD2/3 pathway.

TGFβ has been implicated in the pathogenesis and progression of a number of disease conditions, such as cancer, fibrosis, and immune disorders. In many cases, such conditions are associated with dysregulation of the extracellular matrix (ECM). For these and other reasons, TGFβ has been an attractive therapeutic target for the treatment of immune disorders, various proliferative disorders, and fibrotic conditions. However, observations from preclinical studies, including in rats and dogs, have revealed serious toxicities associated with systemic inhibition of TGFβs in vivo, and to date, there are no TGFβ therapeutics available in the market which are deemed both safe and efficacious.

Dose-limiting toxicities noted with inhibition of the TGFβ pathway have remained a major concern in the development of anti-TGFβ therapies. These include cardiovascular abnormalities, skin lesions, epithelial oral hyperplasia, and gingival bleeding (Vitsky 2009; Lonning 2011; Stauber 2014; Mitra 2020). Although many of these toxicities are either reversible or manageable, the cardiovascular lesions such as inflammation, hemorrhage or hyperplasia in the valves, aortic arch and associated arteries of the heart, are not reversible and therefore continue to be key safety issues when developing TGFβ inhibitors (Stauber 2014; Anderton 2011; Mitra 2020).

Previously, Applicant described a class of monoclonal antibodies that have a novel mechanism of action to modulate growth factor signaling (see, for example, WO 2014/182676, the contents of which are herein incorporated by reference in their entirety). These antibodies were designed to exploit the fact that TGF31 is expressed as latent pro-protein complex comprised of prodomain and growth factor, which requires an activation step that releases the growth factor from the latent complex. Rather than taking the traditional approach of directly targeting the mature growth factor itself post-activation (such as neutralizing antibodies), the novel class of inhibitory antibodies specifically targeted the inactive pro-proprotein complex itself so as to preemptively block the activation step, upstream of ligand-receptor interaction. Without being bound by theory, it was reasoned that this unique mechanism of action should provide advantages for achieving both spatial and temporal benefits in that they act at the source, that is, by targeting the latent proTGFβ1 complex within a disease microenvironment before activation takes place.

Using this approach, further monoclonal antibodies that specifically bind and inhibit the activation step of TGFβ1 (that is, release of mature growth factor from the latent complex) in an isoform-selective manner have been generated (see, WO 2017/156500, the contents of which are herein incorporated by reference in their entirety). Data presented for those antibodies support the notion that isoform-specific inhibition (as opposed to pan-inhibition) of TGFβ may render improved safety profiles of antagonizing TGFβ in vivo. Taking this into consideration, the instant inventors have sought to develop TGFβ1 inhibitors that are both i) isoform-specific; and, ii) capable of broadly targeting multiple TGFβ1 signaling complexes that are associated with different presenting molecules, as therapeutic agents for conditions driven by multifaceted TGFβ1 effects and dysregulation thereof. A non-limiting example of such an isoform-specific inhibitor is a TGFβ1-selective antibody, e.g., Ab4, Ab5, Ab6, Ab21, Ab22, Ab23, Ab24, Ab25, Ab26, Ab27, Ab28, Ab29, Ab30, Ab31, Ab32, Ab33, or Ab34 disclosed herein.

Examples of such antibodies were subsequently described in WO 2018/129329 and PCT/US2019/041373, the contents of each of which are herein incorporated by reference in their entirety. These isoform-specific inhibitory agents demonstrated both efficacy and safety in vivo.

For example, PCT/US2019/041373 discloses that isoform-selective, high affinity antibodies capable of targeting large latent complexes (LLCs) of TGFβ1 may be effective to treat TGFβ1-related indications, such as diseases involving abnormal gene expression (e.g., TGFB1, Acta2, Col1a1, Col3a1, Fn1, Itga11, Lox, Lox12, CCL2 and Mmp2), diseases involving ECM dysregulation (e.g., fibrosis, myelofibrosis and solid tumor), diseases characterized by increased immunosuppressive cells (e.g., Tregs, MDSCs and/or M2 macrophages), diseases involving mesenchymal transition, diseases involving proteases, diseases related to abnormal stem cell proliferation and/or differentiation.

In multiple preclinical tumor models, such TGFβ1 inhibitors were shown to overcome tumor primary resistance (i.e., present before treatment initiation) to an immunotherapy (e.g., checkpoint inhibitors), where the tumor is infiltrated with immunosuppressive cell types, such as regulatory T cells, M2-type macrophages, and/or myeloid-derived suppressive cells (tumor-associated MDSCs). Upon treatment, a reduction in the number of tumor-associated immunosuppressive cells (e.g., MDSCs) and a corresponding increase in the number of anti-tumor effector T cells were observed. In multiple preclinical models, (including tumors co-expressing TGFβ1/3 isoforms), significant and durable antitumor effects were achieved, coupled with survival benefits, when used in conjunction with a checkpoint blockade therapy, suggesting that inhibition of TGFβ1 alone was sufficient to sensitize immunosuppressive tumors to cancer immunotherapy such as checkpoint inhibitors. See, Martin et al. Science Translational Medicine (2020), 12(536): eaay8456.

As of the filing date of this application, the prevailing view of the field as a whole appears to be that it is necessary or advantageous to inhibit multiple isoforms of TGFβ to achieve therapeutic effects, while managing toxicities by careful dosing regimen. Consistent with this premise, numerous groups are developing TGFβ inhibitors that target more than one isoform. These include low molecular weight antagonists of TGFβ receptors, e.g., ALK5 antagonists, such as Galunisertib (LY2157299 monohydrate); monoclonal antibodies (such as neutralizing antibodies) that inhibit all three isoforms (“pan-inhibitor” antibodies) (see, for example, WO 2018/134681); monoclonal antibodies that preferentially inhibit two of the three isoforms (e.g., antibodies against TGFβ1/2 (for example WO 2016/161410) and TGFβ1/3 (for example WO 2006/116002 and WO 2020/051333); integrin inhibitors such as antibodies that bind to αVβ3, αVβ5, αVβ6, αVβ8, α5β1, αIIbβ3, or α8β1 integrins and inhibit downstream activation of TGFβ. e.g., selective inhibition of TGFβ1 and/or TGF33 (e.g., PLN-74809), and engineered molecules (e.g., fusion proteins) such as ligand traps (for example, WO 2018/029367; WO 2018/129331 and WO 2018/158727).

Whilst immune checkpoint inhibitors have become one of the most remarkable success stories of cancer therapy in recent years, these therapies are effective in only a small portion of patient populations (Hedge et al., Immunity. 2020 Jan. 14; 52(1):17-35). As single agents, many immune checkpoint inhibitors typically have response rates of only about 10-35%. An unmet need in cancer immunotherapy has been the limited availability of reliable predictable biomarkers (see, for example, Zhang et al., Front. Med. 2019, 13(1): 32-44, “Monitoring checkpoint inhibitors: predictive biomarkers in immunotherapy” and Arora et al., Adv. Ther. 2019, 36: 2638-2678, “Existing and emerging biomarkers for immune checkpoint immunotherapy in solid tumors). Although traditional tumor biopsy offers valuable information on the disease, possible limitations with biopsy include being invasive, not always feasible for sample collection/access, and potentially not being representative of the whole tumoral landscape. Alternatives to biopsies are being actively explored, including gene expression profiling and noninvasive imaging techniques. Certain serum markers may be useful for diagnostic purposes, but less so for prognostic purposes (see, for example, Zhang et. al.). This has led to the suggestion that blood-based evaluation is likely a poor surrogate of what happens in the tumor microenvironment (TME) (Galon & Bruni, Nature Reviews Drug Discovery, 2019 March; 18(3):197-218 “Approaches to treat immune hot, altered and cold tumors with combination immunotherapies”). There remains a need for better guidance as to both selection of suitable” TGFβ inhibitors tailored to certain patient populations and related therapeutic regimen which may provide improved cancer therapy.

### SUMMARY

The present disclosure relates to compositions comprising TGFβ inhibitors and methods for selecting suitable subjects to treat with TGFβ inhibitors, as well as related methods of treatments and monitoring treatment parameters such as efficacy and target engagement. The disclosure provides better and more targeted therapeutics and treatment modalities, including improved ways of identifying candidates for treatment and/or monitoring treatment efficacy, e.g., patients or patient populations who are likely to benefit from the TGFβ inhibitor therapy. Related methods, including therapeutic regimens, and methods for manufacturing such inhibitors are encompassed herein. The selection of particular subjects and TGFβ inhibitors for therapeutic use is aimed to achieve in vivo efficacy.

More specifically, the present disclosure provides, inter alia, i) enhanced methods for analysis aimed to provide better characterization of the cellular architecture within and surrounding a tumor; ii) improved methods for determining circulatory TGFβ levels aimed to achieve greater accuracy; iii) improved methods for assessing circulating MDSC levels, including identification of LRRC33 as a novel marker for circulating MDSC cells and improved surface markers for identification of mMDSC and gMDSC sub-populations; and iv) additional biomarkers, including p-Smad2, which are useful for predicting and monitoring therapeutic efficacy, as well as determining target engagement. Thus, one or more of these features may be employed as part of diagnostic and/or therapeutic regimen for subjects (e.g., patients) with a condition associated with TGFβ1 dysregulation, such as cancer.

In some embodiments, the pharmaceutical composition and/or treatment regimen disclosed herein comprises a TGFβ inhibitor and one or more additional therapies such as a genotoxic therapy and/or a checkpoint inhibitor therapy. In some embodiments, the one or more additional therapies may be administered in conjunction with the TGFβ inhibitor, either as a separate molecular entity administered separately, as a single formulation (e.g., an admixture), or as part of a single molecular entity, e.g., an engineered multifunctional construct that functions as both a checkpoint inhibitor and a TGFβ inhibitor. In some embodiments, the checkpoint inhibitor therapy is an antibody or an antigen-binding fragment that targets PD-1, PD-L1, CTLA-4, or LAG3. In some embodiments, the genotoxic therapy is a chemotherapy or a radiation therapy. In some embodiments, the TGFβ inhibitor and the one or more additional therapies are administered concurrently, separately, or sequentially. In some embodiments, the TGFβ inhibitor is a TGFβ1 inhibitor, such as apitegromab or Ab6. In some embodiments, the TGFβ inhibitor is any one of the antibodies or antigen-binding fragments disclosed in PCT/JP2015/006323, the content of which is hereby incorporated by reference in its entirety. In some embodiments, the TGFβ inhibitor is GYM329. In some embodiments, the TGFβ inhibitor is an inhibitor of TGFβ1 and TGF32, such as NIS793/XOMA-089 or GC1008. In some embodiments, the TGFβ inhibitor is an inhibitor of TGFβ1 and TGF33, such as M7824 (bintrafusp alpha) or AVID200. In some embodiments, the TGFβ inhibitor is a pan TGFβ inhibitor (i.e., an agent that inhibits TGFβ1, TGF32, and TGFβ3), including GC1008 or derivative thereof, SAR439459, LY3022859, or an agent that blocks a ligand-binding domain of a TGFβ receptor. In some embodiments, the TGFβ inhibitor is an agent that binds the RGD motif present in latent TGFβ1 and/or TGF33, e.g., a low molecular weight (small molecule) compound or an antibody or antigen-binding fragment. In some embodiments, the TGFβ inhibitor is an RNA-based inhibitor of TGFβ1 expression. In some embodiments, the TGFβ inhibitor is a soluble ligand trap such as M7824 (bintrafusp alpha) or AVID200.

In various embodiments, the disclosure provides a method of treating, predicting, and/or monitoring therapeutic efficacy of a TGFβ inhibitor treatment in a subject by measuring or monitoring circulating TGFβ levels (e.g., circulating TGFβ1 levels, e.g., circulating latent TGFβ1 levels). Without being bound by theory, the instant inventors have discovered that administering a TGFβ inhibitor to a subject increases TGFβ levels in the subject's blood, possibly due to an accumulation of latent TGFβ as a result of inhibiting the TGFβ activation pathway. Thus, the disclosure contemplates the use of circulating TGFβ, e.g., from a blood sample obtained from a subject, as a biomarker to determine and monitor therapeutic efficacy and/or target engagement, and to guide decisions on treatment. The terms circulating and circulatory (as in “circulating TGFβ” and “circulatory TGFβ”) may be used interchangeably.

In some embodiments, the disclosure provides a method of treating, predicting, and/or monitoring therapeutic efficacy of a TGFβ inhibitor treatment in a subject, the method comprising (i) determining a level of circulating TGFβ in the subject prior to administering a TGFβ inhibitor; (ii) administering to the subject a therapeutically effective amount of the TGFβ inhibitor; and (iii) determining a level of circulating TGFβ in the subject after administration, wherein an increase in circulating TGFβ after the administration as compared to before the administration indicates therapeutic efficacy. In some embodiments, the increase is at least 1.5-fold, at least 2-fold, at least 2.5-fold, at least 3-fold, at least 4-fold, at least 5-fold, or more. In some embodiments, the treatment alters the level of circulating TGFβ. In some embodiments, continued treatment is contingent on an observed increase in circulating TGFβ.

In some embodiments, the disclosure encompasses a method of determining target engagement and/or therapeutic efficacy of a TGFβ inhibitor treatment in a subject, wherein the treatment comprises (i) determining the circulating TGFβ level in a sample obtained from the subject prior to administering the TGFβ inhibitor; (ii) administering a first dose of the TGFβ inhibitor to the subject; and (iii) determining the circulating TGFβ level in a sample obtained from the subject after the administration, wherein an increase in the circulating TGFβ level after the administration as compared to before the administration is indicative of target engagement and/or therapeutic efficacy. In some embodiments, the method comprises administering to the subject a second dose of the TGFβ inhibitor if an increase in the circulating TGFβ level after the administration as compared to before the administration is observed. In some embodiments, the treatment is continued if an increase in the circulating TGFβ level after the administration as compared to before the administration is observed. In some embodiments, the increase is at least 1.5-fold, at least 2-fold, at least 2.5-fold, at least 3-fold, at least 4-fold, at least 5-fold, or more.

In any one of the previous embodiments, the treatment may further comprise administering to the subject one or more additional therapies, e.g., a genotoxic therapy and/or immunotherapy, wherein the one or more additional therapies are administered concurrently (e.g., simultaneously), separately, or sequentially. For instance, the additional therapy may comprise a checkpoint inhibitor therapy.

In some embodiments, the disclosure provides an improved method for measuring a circulating TGFβ level from a blood sample or a sample derived from blood, the method comprising processing the sample at a temperature of 2-8 DC in a sample tube comprising an anticoagulant. In some embodiments, the anticoagulant is citrate-theophylline-adenosine-dipyridamole (CTAD). In some embodiments, the tube is coated with a citrate-theophylline-adenosine-dipyridamole (CTAD) solution, a 0.11 M buffered trisodium citrate solution, 15 M theophylline, 3.7 M adenosine, and 0.198 M dipyridamole, wherein the solution has a pH of 5.0. In some embodiments, the sample processing comprises one or more centrifugation steps at a speed of greater than 100×g and/or one or more centrifugation steps at a speed of below 15000×g. In some embodiments, the sample processing comprises a centrifugation protocol comprising: i) a first step of 10 minutes at 150×g and a second step of 20 minutes at 2500×g; or ii) a first step of 10 minutes at 2500×g and a second step of 20 minutes at 2500×g; or iii) a first step of 10 minutes at 1500×g and a second step of 5 minutes at 12000×g. In some embodiments, the method comprises analyzing a level of plasma factor 4 (PF4) in the same sample from which the circulating TGFβ level is determined, such thatthe PF4 level provides quality control for the sample. In some embodiments, a sample is only used to determine a circulating TGFβ level if the PF4 level in the same sample is below a concentration indicative of plasma activation. In some embodiments, a PF4 level of greaterthan 500 ng/ml is indicative of plasma activation. Thus, in some embodiments, a sample is used to assess a circulating TGFβ level only if the sample has a PF4 level of 500 ng/ml or less.

In some embodiments, the disclosure provides a method of treating, predicting, and/or monitoring therapeutic efficacy of a TGFβ inhibitor treatment in a subject, the method comprising (i) determining a level of phosphorylated Smad2 (P-Smad2) nuclear translocation in a tumor sample obtained from the subject prior to administering a TGFβ inhibitor (pre-treatment tumor sample); (ii) administering to the subject one or more doses of the TGFβ inhibitor; and (iii) determining a level of P-Smad2 nuclear translocation in a tumor sample obtained from the subject after the administration (post-treatment tumor sample); wherein a decrease in P-Smad2 nuclear translocation after the administration as compared to before the administration indicates therapeutic efficacy. In some embodiments, the treatment is continued if a decrease in P-Smad2 nuclear translocation is observed in the post-treatment tumor sample. In some embodiments, the decrease is by at least 1.3-fold, at least 1.5-fold, at least 2-fold, at least 3-fold, at least 4-fold, at least 5-fold. In some embodiments, the level of P-Smad2 nuclear translocation is determined by nuclear masking.

In various embodiments, the disclosure provided herein involves the use of circulating MDSC levels as a predictive biomarker to improve the diagnosis, monitoring, patient selection, prognosis, and/or continued treatment of a subject being administered a TGFβ inhibitor (e.g., a TGFβ1-selective inhibitor such as Ab6) by monitoring circulating MDSC levels. In some embodiments, the disclosure also encompasses methods of determining therapeutic efficacy and therapeutic agents (e.g., compositions) or regiments for use in subjects with cancer by measuring levels of circulating MDSCs. Without being bound by theory, the instant inventors have discovered that reversal of or overcoming an immunosuppressive phenotype, e.g., in a cancer or related condition that manifests dysregulation of the ECM, such as by administration of a TGFβ inhibitor, can be indicated by analyzing circulating MDSC levels, e.g., in a sample obtained from a subject, e.g., in blood or a blood component, e.g., prior to the time point when a reduction in tumor volume or other biomarkers might be used to confirm treatment efficacy. In some embodiments, circulatory MDSCs are characterized by cell-surface expression of LRRC33. In some embodiments, sub-populations of circulatory MDSCs are measured, such as m-MDSCs and/or g-MDSCs. The terms circulating and circulatory (as in “circulating MDSCs” and “circulatory MDSCs”) may be used interchangeably.

Tumor-associated MDSC cells may contribute to TGFβ31-mediated immunosuppression in the tumor microenvironment. Previously, Applicant showed that MDSCs were indeed enriched in solid tumors and that inhibition of TGF31 in conjunction with a checkpoint inhibitor treatment significantly reduced intratumoral MDSCs, which correlated with slowed tumor growth and, in some cases, achieved complete regression in multiple preclinical tumor models (PCT/US2019/041373). In these efficacy studies, effectiveness of such combination therapy was observed over the course of weeks to months (for example, 6-12 weeks) by monitoring tumor growth. Tumor biopsy may reveal an immune profile of a tumor microenvironment (TME); however, in addition to being invasive, biopsy-based information may be inaccurate or skewed because tumor-infiltrating lymphocytes (TILs) may not be uniformly present within the whole tumor, and therefore, depending on which portion of the tumor is sampled by biopsy, results may vary. To overcome the limitations of biopsy-based analyses, data presented herein now establish the correlation between tumor-associated (e.g., intratumoral) MDSC levels and circulatory MDSC levels, raising the possibility that MDSCs measured in blood samples (e.g., whole blood or a blood component, e.g., PBMCs) may serve as a surrogate to more accurately predict patient populations that are likely to benefit from certain therapeutic regimens. Furthermore, evidence suggests the degree of tumor burden (e.g., the size of tumor) correlates with the relative level of circulating MDSCs in the subject bearing the tumor. Therefore, by monitoring circulating MDSC levels in a subject after receiving the therapy, response to the therapy (e.g., therapeutic effects) may be evaluated without the need for invasive biopsies, and results may be obtained sooner than conventional methods. Additionally, more recent findings presented herein identify, inter alia, LRRC33 as a novel cell-surface marker for MDSCs in circulation (e.g., blood samples). This observation raises the possibility that surface LRRC33 expression may be used as a blood-based predictive biomarker.

In various embodiments, the methods disclosed herein employ circulating MDSCs as an early biomarker to predict the efficacy of combination therapy comprising a TGFβ inhibitor. Data disclosed herein show that after TGF31 inhibitor treatment, there is a marked reduction in circulating MDSC levels relative to baseline, which can be a reduction in mMDSC levels and/or gMDSC levels. Such circulating MDSC levels can be measured in blood or a blood component, which can be detected well before antitumor efficacy outcome can readily be obtained, in some cases shortening the timeline by weeks. Thus, the disclosure provides the use of circulating MDSCs as a predictive biomarker for the patient's responsiveness to a cancer therapy, e.g., a combination therapy. In related aspects of the disclosure provided herein, the level of circulating MDSC cells may be determined within 1-10 weeks, e.g., 3-6 weeks, following administration of a dose of a TGFβ inhibitor, optionally within 3 weeks or at about 3 weeks following administration of the dose of TGFβ inhibitor. In some embodiments, the level of circulating MDSC cells may be determined within 2 weeks following administration of the dose of TGFβ inhibitor. In some embodiments, the level of circulating MDSC cells may be determined at about 10 days following administration of the dose of TGFβ inhibitor.

Cancer immunotherapy may harness or enhance the body's immunity to combat cancer. Without being bound by theory, it is contemplated that low levels of circulating MDSCs in subjects with cancer indicate that the body has retained or restored disease-fighting immunity (e.g., antitumor activity), more specifically, lymphocytes such as CD8+ T cells, which can be mobilized to attack malignant cells. Thus, reduced levels of circulating MDSCs upon TGFβ inhibitor treatment may indicate pharmacodynamic effects of TGFβ inhibition (e.g., TGFβ1 inhibition) and serve as an early predictive biomarker for therapeutic efficacy when treated with a cancer therapy such as checkpoint inhibitors.

Where cancer patients receive a combination therapy comprising a cancer therapy (such as checkpoint inhibitor) and a TGFβ inhibitor that is not selective for TGFβ1 (non-selective TGFβ inhibitor), there may be a greater risk of toxicity. To mitigate or manage such risk, non-selective TGFβ inhibitor may be administered infrequently or intermittently, for example on an “as-needed” basis. For example, circulating MDSC levels may be monitored periodically in order to determine that the effects of overcoming immunosuppression are sufficiently maintained, so as to ensure antitumor effects of the cancer therapy. During the course of cancer treatment, if MDSC levels become elevated, this may indicate that the patient may benefit from additional dose(s) of a TGFβ inhibitor. Such approach may help reduce unnecessary risk and adverse events associated with over-exposure to a TGFβ inhibitor, particularly a non-TGFβ1 selective inhibitor. In some embodiments, the TGFβ inhibitor targets TGFβ1/2 signaling. In some embodiments, the TGFβ inhibitor targets TGFβ1/3 signaling. In some embodiments, the TGFβ inhibitor targets TGFβ1/2/3 signaling.

In some embodiments, disclosed herein are methods of treating cancer (also described herein in the context of compositions for use in treating cancer or cancer treatments). Also disclosed are methods of predicting, determining, or monitoring therapeutic efficacy in subjects with cancer, e.g., monitoring a patient's responsiveness to treatment and/or making continued treatment decisions based on the monitored parameters. In some embodiments, the cancer is an immune excluded cancer and/or a myeloproliferative disorder, wherein the myeloproliferative disorder may be myelofibrosis. In some embodiments, the cancer is a TGFβ1-positive cancer. The TGFβ1-positive cancer may co-express TGFβ1, TGF32, and/or TGF33. The TGFβ1-positive cancer may be a TGFβ1-dominant tumor. The TGFβ1-positive cancer may be a TGFβ1-dominant tumor and may co-express TGFβ1, TGF32, and/or TGF33. For instance, the TGFβ1-positive cancer may be a TGFβ1-dominant tumor and may co-express TGFβ1 and TGF32. As another example, The TGFβ1-positive cancer may be a TGFβ1-dominant tumor and may co-express TGFβ1 and TGF33. Such cancer includes advanced cancer, e.g., metastatic cancer (e.g., metastatic solid tumors) and cancer with a locally advanced tumor (e.g., locally advanced solid tumors). In some embodiments, the treatment comprises administering to the subject a TGFβ inhibitor in an amount sufficient to reduce circulating MDSC levels.

In some embodiments, the disclosure encompasses a method of predicting or determining therapeutic efficacy in a subject having cancer comprising the steps of determining circulating MDSC levels in the subject prior to administering a TGFβ inhibitor (alone or in conjunction with a cancer therapy), administering to the subject a therapeutically effective amount of the TGFβ inhibitor (alone or in conjunction with a cancer therapy), and determining circulating MDSC levels in the subject after the administration, wherein a reduction in circulating MDSC levels after administration, as compared to circulating MDSC levels before administration, predicts therapeutic efficacy.

In some embodiments, the disclosure encompasses a method of determining therapeutic efficacy of a TGFβ inhibitor treatment in a subject, wherein the treatment comprises (i) determining the circulating MDSC level in a sample obtained from the subject prior to administering the TGFβ inhibitor; (ii) administering a first dose of the TGFβ inhibitor to the subject; and (iii) determining the circulating MDSC level in a sample obtained from the subject after the administration, wherein a reduction in the circulating MDSC level after the administration as compared to before the administration is indicative of therapeutic efficacy. In some embodiments, the method comprises administering to the subject a second dose of the TGFβ inhibitor if a reduction in the circulating MDSC level after the administration as compared to before the administration is observed. In some embodiments, the treatment is continued if a reduction in the circulating MDSC level after the administration as compared to before the administration is observed.

In some embodiments, the disclosure encompasses a combination therapy comprising a dose of a TGFβ inhibitor and a checkpoint inhibitor therapy and/or a genotoxic therapy for use in the treatment of cancer, wherein the treatment comprises concurrent (e.g., simultaneous), separate, or sequential administration of a dose of the TGFβ inhibitor, wherein a reduction in circulating MDSC level after the administration as compared to before the administration has been determined.

In some embodiments, the disclosure encompasses a TGFβ inhibitor for use in the treatment of cancer in a subject, wherein the subject is administered a dose of the TGFβ inhibitor, and wherein the TGFβ inhibitor reduces or reverses immune suppression in the cancer, wherein said reduced or reversed immune suppression has been determined by a reduction in the circulating MDSC level in the subject measured after the administration of the TGFβ inhibitor as compared to the circulating MDSC level measured in the subject prior to administering the dose of the TGFβ inhibitor.

In some embodiments, the disclosure encompasses a method of treating advanced cancer in a human subject comprising the steps of selecting a subject with advanced cancer and administering a combination therapy comprising a TGFβ inhibitor and a checkpoint inhibitor therapy, wherein the advanced cancer comprises a locally advanced tumor and/or metastatic cancer with primary resistance to a checkpoint inhibitor therapy, wherein the subject has elevated circulating MDSC levels. In some embodiments, the combination therapy reduces the circulating MDSC level in the subject. In some embodiments, continued treatment is contingent on an observed reduction in the subject's circulating MDSC level.

In various embodiments, the circulating MDSC level may be a level of mMDSC and/or gMDSC. In some embodiments, mMDSCs are identified by cell surface markers of CD11b+, HLA−DR−/low, CD14+, CD15−, CD33+/high, and CD66b−. In some embodiments, gMDSCs are identified by cell surface markers of CD11 b+, HLA−DR−, CD14−, CD15+, CD33+/low, and CD66b+. In some embodiments, the reduction in a circulating MDSC level (e.g., circulating mMDSC and/or circulating gmMDSC) may be a reduction of at least 10%.

In some embodiments, the disclosure encompasses a method of treating, predicting, determining, and/or monitoring therapeutic efficacy of a cancer treatment in a subject administered a TGFβ inhibitor alone or in combination with one or more additional cancer therapies (e.g., a checkpoint inhibitor therapy and/or a genotoxic therapy), the method comprising the steps of: (i) obtaining a pre-treatment biopsy sample from the subject, (ii) determining a level of tumor-associated CD8+ cells in the pre-treatment biopsy sample, (iii) administering the treatment to the subject, (iv) obtaining a post-treatment biopsy sample from the subject, and (v) determining a level of tumor-associated CD8+ cells in the post-treatment biopsy sample, wherein the levels of tumor-associated CD8+ cells in the biopsy samples are determined by immunohistochemical analysis of individual tumor nests within the tumor.

In some embodiments, a subject having an immune inflamed tumor characterized by a pre-treatment biopsy sample having greater than 5% CD8+ cells in individual tumor nests is selected for the treatment. In some embodiments, the immune inflamed tumor is characterized by having greater than 5% CD8+ cells in greater than 50% of the individual tumor nests detected.

In some embodiments, a subject having an immune excluded tumor characterized by a pre-treatment biopsy sample having less than 5% intratumor CD8+ cells and greater than 5% margin CD8+ cells is selected for treatment. In some embodiments, the immune excluded tumor is characterized by having less than 5% CD8+ cells in individual tumor nests. In some embodiments, the immune excluded tumor is characterized by having less than 5% CD8+ cells in greater than 50% of the individual tumor nests detected.

In some embodiments, therapeutic efficacy can be monitored by comparing the levels of CD8+ cells in the pre-treatment biopsy sample and the post-treatment biopsy sample such that a change in CD8+ level in the post-treatment biopsy sample as compared to the CD8+ level in the pre-treatment indicates therapeutic efficacy. In some embodiments, an increase of intratumor CD8+ cells (e.g., an increase of CD8+ cells inside tumor nests) in the post-treatment biopsy sample as compared to the pre-treatment biopsy sample indicates therapeutic efficacy.

In some embodiments, any one of the biomarkers disclosed herein may be used in conjunction with one or more of the other biomarkers provided herein. For example, circulating TGFβ may be monitored in combination with one or more biomarkers disclosed herein, e.g., circulating MDSC, tumor-associated CD8+ cell, intratumor or circulating cytokines, and/or p-Smad2 nuclear translocation. In some embodiments, treatment efficacy and/or continued treatment may be contingent on observed changes in two or more sets of biomarkers.

In various embodiments, the methods and compositions for use of the present disclosure comprise treating or selecting for treatment a subject having a cancer, wherein the cancer may be a highly metastatic cancer and/or a solid cancer. In some embodiments, the subject has melanoma, triple-negative breast cancer, HER2-positive breast cancer colorectal cancer (e.g., microsatellite stable-colorectal cancer, lung cancer (e.g., non-small cell lung cancer or small cell lung cancer), pancreatic cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer (e.g., gastric cancer), or thyroid cancer.

In some embodiments, the methods and compositions for use of the present disclosure comprise treating or selecting for treatment a subject having a cancer that is resistant to immunotherapy. The subject may be treatment-naïve (e.g., has not previously received a cancer therapy), may have primary resistance to an immunotherapy (i.e., resistance is present before treatment initiation), or may have acquired resistance to an immunotherapy (i.e., resistance as a result of at least one dose of treatment). In some embodiments, the immunotherapy is a checkpoint inhibitor therapy, e.g., an anti-PD-1 or anti-PD-L1 antibody.

In some embodiments, the methods and compositions for use according to the present disclosure encompass providing treatment to a treatment-naïve subject. In some embodiments, the methods and compositions for use according to the present disclosure encompass providing treatment to a subject who has previously received a cancer therapy or who is currently receiving cancer therapy. A previous cancer therapy may be the same cancer therapy to be administered according to the invention. The cancer therapy may be checkpoint inhibitor (CPI) therapy. In some embodiments, the methods and compositions for use according to the present disclosure encompass providing treatment to a cancer subject wherein the cancer is or is suspected of being immune suppressive (e.g., having a tumor with an immune excluded or immunosuppressive phenotype).

In some embodiments, the methods and compositions for use according to the present disclosure encompass providing treatment to a subject having a cancer with a high response rate to checkpoint inhibitor therapy (e.g., overall response rate of greater than 30%, greater 40%, greater than 50%, or greater). Examples of cancer with high response rates to checkpoint inhibitor therapy include, but are not limited to, microsatellite instability-colorectal cancer (MSI-CRC), renal cell carcinoma (RCC), melanoma (e.g., metastatic melanoma), Hodgkin's lymphoma, NSCLC, cancer with high microsatellite instability (MSI-H), cancer with mismatch repair deficiency (dMMR), primary mediastinal large B-cell lymphoma (PMBCL), and Merkel cell carcinoma (e.g., as reported in Haslam et al., JAMA Network Open. 2019; 2(5): e192535).

In some embodiments, the methods and compositions for use according to the present disclosure encompass providing treatment to a subject having a cancer with a low response rate to checkpoint inhibitor therapy (e.g., overall response rate of 30% or less, 20% or less, or 10%, or less). In some embodiments, the subject may be treatment-naïve. In some embodiments, the subject may be resistant to checkpoint inhibitor therapy. Examples of cancer with low response rates to checkpoint inhibitor therapy include, but are not limited to, ovarian cancer, gastric cancer, and triple-negative breast cancer.

In various embodiments, the methods and compositions for use according to the present disclosure encompass providing treatment to a subject having a solid cancer. In some embodiments, the solid cancer is selected from melanoma (e.g., metastatic melanoma), renal cell carcinoma, breast cancer, e.g., triple-negative breast cancer, HER2-positive breast cancer, colorectal cancer, e.g., microsatellite stable-colorectal cancer and colon adenocarcinoma, lung cancer (e.g., metastatic non-small cell lung cancer, small cell lung cancer), esophageal cancer, pancreatic cancer, bladder cancer, kidney cancer, e.g., transitional cell carcinoma, renal sarcoma, and renal cell carcinoma (RCC), including clear cell RCC, papillary RCC, chromophobe RCC, collecting duct RCC, or unclassified RCC, uterine cancer, e.g., uterine corpus endometrial carcinoma, prostate cancer, stomach cancer (e.g., gastric cancer), head and neck cancer, e.g., head and neck squamous cell cancer, urothelial carcinoma, hepatocellular carcinoma, thyroid cancer, or tenosynovial giant cell tumor (TGCT).

In some embodiments, a TGFβ inhibitor of the present disclosure may be used to treat, including to improve rates or ratios of complete verses partial responses among the responders of a cancer therapy. Typically, even in cancer types where response rates to a cancer therapy (e.g., a checkpoint inhibitor therapy) are relatively high (e.g., ≥30% responders), complete response rates are low. The TGFβ inhibitors of the present disclosure may therefore be used to increase the fraction of complete responders within the responder population. In preferred embodiments, the TGFβ inhibitor is Ab6.

In various embodiments, the TGFβ inhibitor of the present disclosure does not inhibit TGF32 signaling at a therapeutically effective dose. In some embodiments, the TGFβ inhibitor does not inhibit TGF33 signaling at a therapeutically effective dose. In some embodiments, the TGFβ inhibitor does not inhibit TGF32 signaling and TGF33 signaling at a therapeutically effective dose.

In various embodiments, the TGFβ inhibitor is a TGFβ1-selective inhibitor. In some embodiments, the TGFβ inhibitor may bind TGFβ1 with an affinity of 0.5 nM or greater (KD<0.5 nM) with a dissociation rate of no more than 10.0E-4 (1/s) as measured by SPR. More preferably, the TGFβ inhibitor is an activation inhibitor of TGFβ1. For example, the activation inhibitor may be a monoclonal antibody or an antigen-binding fragment thereof that binds the latent lasso region of a latent TGFβ1 complex. In some embodiments, the TGFβ inhibitor is Ab4, Ab5, Ab21, Ab22, Ab23, Ab24, Ab25, Ab26, Ab27, Ab28, Ab29, Ab30, Ab31, Ab32, Ab33, or Ab34. Most preferably, the TGFβ inhibitor is Ab6 or a variant thereof (e.g., a variant of Ab6 as used herein is one that retains at least 80%, 90%, 95% or greater sequence similarity to Ab6 and/or retains one or more binding and/or therapeutic properties of Ab6, so as to achieve a desired therapeutic effect).

In various embodiments, the methods and compositions for use disclosed herein comprise use of a TGFβ inhibitor disclosed herein in conjunction (e.g., in combination) with a checkpoint inhibitor and/or a genotoxic therapy, wherein the checkpoint inhibitor is an anti-PD-1 antibody, anti-PD-L1 antibody, anti-CTLA-4-antibody, anti-LAG3 antibody, or an antigen-binding fragment thereof; and/or the genotoxic therapy is a chemotherapy or a radiation therapy, wherein optionally, the chemotherapy is a PARP inhibitor therapy.

In various embodiments, the methods and compositions for use disclosed herein comprise use of a TGFβ inhibitor disclosed herein in conjunction with at least one additional therapy. In some embodiments, the at least one additional therapy is a cancer therapy, such as an immunotherapy, a genotoxic therapy, including chemotherapy and radiation therapy (including radiotherapeutic agents), an engineered immune cell therapy (e.g., CAR-T therapy), a cancer vaccine therapy, and/or an oncolytic viral therapy. In some embodiments, the at least one additional therapy is chemotherapy or radiation therapy (including radiotherapeutic agents). In some embodiments, the at least one additional cancer therapy is a checkpoint inhibitor therapy. In some embodiments, the checkpoint inhibitor may comprise an agent targeting programmed cell death protein 1 (PD-1) or programmed cell death protein 1 ligand (PD-L1). For instance, the checkpoint inhibitor may comprise an anti-PD-1 or anti-PD-L1 antibody. In some embodiments, the TGFβ inhibitors disclosed herein may be used in conjunction with at least one additional therapy selected from: a PD-1 antagonist (e.g., a PD-1 antibody), a PDL1 antagonist (e.g., a PDL1 antibody), a PD-L1 or PDL2 fusion protein, a CTLA4 antagonist (e.g., a CTLA4 antibody), a GITR agonist e.g., a GITR antibody), an anti-ICOS antibody, an anti-ICOSL antibody, an anti-B7H3 antibody, an anti-B7H4 antibody, an anti-TIM3 antibody, an anti-LAG3 antibody, an anti-OX40 antibody (OX40 agonist), an anti-CD27 antibody, an anti-CD70 antibody, an anti-CD47 antibody, an anti-41BB antibody, an anti-PD-1 antibody, an anti-CD20 antibody, an anti-CD3 antibody, an anti-PD-1/anti-PDL1 bispecific or multispecific antibody, an anti-CD3/anti-CD20 bispecific or multispecific antibody, an anti-HER2 antibody, an anti-CD79b antibody, an anti-CD47 antibody, an antibody that binds T cell immunoglobulin and ITIM domain protein (TIGIT), an anti-ST2 antibody, an anti-beta7 integrin (e.g., an anti-alpha4-beta7 integrin and/or alphaE beta7 integrin), a CDK inhibitor, an oncolytic virus, an indoleamine 2,3-dioxygenase (IDO) inhibitor, and/or a PARP inhibitor.

### BRIEF DESCRIPTION OF THE FIGURES

FIG. 1 shows tumor MDSC levels measured in MBT-2 tumors.

FIG. 2 shows tumor volume and circulating G-MDSC and M-MDSC levels in MBT-2 mice.

FIG. 3 shows tumor volume in MBT-2 mice across treatment groups.

FIG. 4 shows baseline level of circulating MDSCs in non-tumor bearing mice.

FIG. 5 shows levels of circulating MDSCs in tumor-bearing mice.

FIG. 6 shows a comparison of circulating MDSC levels in non-tumor bearing mice and tumor-bearing mice.

FIG. 7A shows a comparison of circulating M-MDSC and G-MDSC levels on days 3-10; FIG. 7B shows time-course of changes in circulating M-MDSC and G-MDSC levels from days 3-10.

FIG. 8 is a plot of circulating MDSC level and tumor volume on day 10 across treatment groups.

FIG. 9A shows tumor MDSC levels in different treatment groups; FIG. 9B shows a comparison of circulating G-MDSC levels and tumor MDSC levels on day 10 across treatment groups.

FIG. 10 shows correlation of tumor MDSC levels to circulating MDSC levels.

FIG. 11 shows tumor G-MDSC and tumor CD8+ cells across all treatment groups.

FIG. 12A shows circulating gMDSC and mMDSC levels in whole blood of mice bearing MBT2 tumors; FIG. 12B shows intratumoral gMDSC and mMDSC levels in mice bearing MBT2 tumors.

FIG. 13A shows circulating TGF31 levels (pg/mL) in MBT-2 mice; FIG. 13B shows plasma levels of Ab6 (μg/mL, left) and TGF31 (pg/mL, right); FIG. 13C shows correlation of plasma levels of Ab6 (μg/mL) and TGF31 (pg/mL) in MBT-2 mice treated with AB6 alone or in combination with an anti-PD1 antibody.

FIG. 14 shows plasma platelet factor 4 levels (ng/mL) in MBT-2 mice (right) and sample outliers as determined by interquartile range (left).

FIG. 15 shows identified sample outliers (left) and outlier-corrected levels (pg/mL) of circulatory TGFβ1 (right).

FIG. 16 shows circulatory TGFβ levels in NHP following a single dose of Ab6.

FIG. 17 shows circulatory TGFβ levels in rats following a single dose of Ab6.

FIG. 18 shows an exemplary sample collection and processing method for evaluating circulating TGFβ1 levels in blood.

FIG. 19A shows circulating TGFβ1 levels in blood samples as evaluated under various sample processing conditions.

FIG. 19B shows platelet factor 4 (PF4) levels in blood samples as evaluated under various sample processing conditions.

FIG. 20 shows correlation of circulating TGFβ1 levels and PF4 levels in blood samples as evaluated under various sample processing conditions.

FIG. 21A shows PF4 levels in blood samples as evaluated under various sample processing conditions;

FIG. 21B and FIG. 21C show exemplary outlier analysis based on measurement of PF4 levels.

FIG. 22 shows PF4 vs. TGFβ1 levels pre-dose and 1 hour post-dose.

FIG. 23A shows fold change in TGFβ levels over time in subjects treated with 80-240 mg of Ab6; FIG. 23B shows fold change in TGFβ levels over time in subjects treated with 800 mg of Ab6; FIG. 23C shows fold change in TGFβ levels over time in subjects treated with 1600 mg of Ab6.

FIG. 24 shows a P-Smad2 IHC analysis of melanoma samples.

FIG. 25 shows pSmad-2 signaling in MBT2 tumors following treat with Ab6-mlgG1.

FIG. 26A shows tissue compartment data of bladder cancer samples; FIG. 26B shows tissue compartment data of melanoma samples.

FIG. 27 shows density of CD8+ cells in bladder cancer samples as analyzed based on tumor nest.

FIG. 28 shows immune phenotype analysis of a single bladder cancer sample based on density of CD8+ cells measured in tumor nests.

FIG. 29A shows average percentages of CD8+ cells and immune phenotyping in bladder cancer and melanoma samples, as analyzed by tumor compartments (left) and tumor nests (right); underlined phenotype reflects differences between analyses; FIG. 29B shows average percentages of CD8+ cells and immune phenotyping in bladder cancer and melanoma samples, as analyzed by tumor compartments (left) and tumor nests (right); underlined phenotype reflects differences between analyses; FIG. 29C shows tumor nest data and immune phenotyping for individual tumor nests identified from bladder cancer samples; FIG. 29D shows tumor nest CD8+ data and immune phenotyping for bladder cancer and melanoma samples; FIG. 29E shows percent CD8+ cells in tumor, tumor margin, and stroma compartments of commercially available bladder cancer samples.

FIG. 30A shows representative CD8+ staining in bladder cancer samples; FIG. 30B shows subdivision of CD8+ staining in the tumor margin compartment; FIG. 30C shows subdivision of CD8+ staining in the tumor margin compartment of a bladder sample.

FIG. 31 shows comparison of compartment CD8+ ratio and absolute percent CD8 positivity.

FIG. 32 shows comparison of CD8+ cell density and absolute percent CD8 positivity.

FIG. 33 shows tumor depth of bladder samples.

FIG. 34 shows CD8 density in a melanoma sample.

FIGS. 35A-c show exemplary analysis of MDSC by signal filtering.

FIGS. 36A-C shows identification of tumor MDSC populations in various solid cancer samples.

FIGS. 37A-C shows analysis of gMDSC and mMDSC populations in various solid cancer samples.

FIG. 38 shows a schematic of an exemplary pathology analysis of tumor tissue sample.

FIG. 39 shows a schematic of an exemplary pathology analysis of tumor tissue sample.

FIG. 40 shows a schematic of an exemplary TGFβ inhibitor treatment regimen.

FIG. 41 illustrates identification of three binding regions (Region 1, Region 2, and Region 3) following statistical analyses. Region 1 overlaps with a region called “Latency Lasso” within the prodomain of proTGFβ1, while Regions 2 and 3 are within the growth factor domain. FIG. 41 discloses SEQ ID NO: 172.

FIG. 42 depicts various domains and motifs of proTGFβ1, relative to the three binding regions involved in Ab6 binding. Sequence alignment among the three isoforms is also provided. FIG. 42 discloses SEQ ID NOS 142, 173-174, 138, 175-176, 141, and 177-178, respectively, in order of appearance.

### DETAILED DESCRIPTION OF CERTAIN EMBODIMENTS

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

Advanced cancer, advanced malignancy: The term “advanced cancer” or “advanced malignancy” as used herein has the meaning understood in the pertinent art, e.g., as understood by oncologists in the context of diagnosing or treating subjects/patients with cancer. Advanced malignancy with a solid tumor can be locally advanced or metastatic. The term “locally advanced cancer” is used to describe a cancer (e.g., tumor) that has grown outside the organ it started in but has not yet spread to distant parts of the body. Thus, the term includes cancer that has spread from where it started to nearby tissue or lymph nodes. By contrast, “metastatic cancer” is a cancer that has spread from the part of the body where it started (the primary site) to other parts (e.g., distant parts) of the body.

Affinity: Affinity is the strength of binding of a molecule (such as an antibody) to its ligand (such as an antigen). It is typically measured and reported by the equilibrium dissociation constant (K_{D}). In the context of antibody-antigen interactions, K_{D} is the ratio of the antibody dissociation rate (“off rate” or K_{off}), how quickly it dissociates from its antigen, to the antibody association rate (“on rate” or Kₒₙ) of the antibody, how quickly it binds to its antigen. For example, an antibody with an affinity of ≤5 nM has a K_{D} value that is 5 nM or lower (i.e., 5 nM or higher affinity) determined by a suitable in vitro binding assay. Suitable in vitro assays can be used to measure K_{D} values of an antibody for its antigen, such as Biolayer Interferometry (BLI) and Solution Equilibrium Titration (e.g., MSD-SET). In a preferred embodiment, affinity is measured by surface plasmon resonance (e.g., Biacore®). An antibody with a suitable affinity in a surface plasmon resonance assay may have, e.g., a K_{D} of at most about 1 nM, e.g., at most about 0.5 nM, e.g., at most about 0.5, 0.4, 0.3, 0.2, 0.15 nM, or less.

Antibody: The term “antibody” encompasses any naturally-occurring, recombinant, modified or engineered immunoglobulin or immunoglobulin-like structure or antigen-binding fragment or portion thereof, or derivative thereof, as further described elsewhere herein. Thus, the term refers to an immunoglobulin molecule that specifically binds to a target antigen, and includes, for instance, chimeric, humanized, fully human, and multispecific antibodies (including bispecific antibodies). An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. Antibodies, or antigen binding portions thereof, can be produced in hybridomas, by recombinant DNA techniques, or by enzymatic or chemical cleavage of intact antibodies. The term antibodies, as used herein, includes monoclonal antibodies, multispecific antibodies such as bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), respectively. In some embodiments, the term also encompasses peptibodies.

Antigen: The term “antigen” broadly includes any molecules comprising an antigenic determinant within a binding region(s) to which an antibody or a fragment specifically binds. An antigen can be a single-unit molecule (such as a protein monomer or a fragment) or a complex comprised of multiple components. An antigen provides an epitope, e.g., a molecule or a portion of a molecule, or a complex of molecules or portions of molecules, capable of being bound by a selective binding agent, such as an antigen binding protein (including, e.g., an antibody). Thus, a selective binding agent may specifically bind to an antigen that is formed by two or more components in a complex. In some embodiments, the antigen is capable of being used in an animal to produce antibodies capable of binding to that antigen. An antigen can possess one or more epitopes that are capable of interacting with different antigen binding proteins, e.g., antibodies. In the context of the present disclosure, a suitable antigen is a complex (e.g., multimeric complex comprised of multiple components in association) containing a proTGF dimer in association with a presenting molecule. Each monomer of the proTGF dimer comprises a prodomain and a growth factor domain, separated by a furin cleavage sequence. Two such monomers form the proTGF dimer complex. This in turn is covalently associated with a presenting molecule via disulfide bonds, which involve a cysteine residue present near the N-terminus of each of the proTGF monomer. This multi-complex formed by a proTGF dimer bound to a presenting molecule is generally referred to as a large latent complex. An antigen complex suitable for screening antibodies or antigen-binding fragments, for example, includes a presenting molecule component of a large latent complex. Such presenting molecule component may be a full-length presenting molecule or a fragment(s) thereof. Minimum required portions of the presenting molecule typically contain at least 50 amino acids, but more preferably at least 100 amino acids of the presenting molecule polypeptide, which comprises two cysteine residues capable of forming covalent bonds with the proTGFβ1 dimer.

Antigen-binding portion/fragment: The terms “antigen-binding portion” or “antigen-binding fragment” of an antibody, as used herein, refers to one or more fragments of an antibody that retain the ability to specifically bind to an antigen (e.g., TGFβ1). Antigen binding portions include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments, a monovalent fragment consisting of the VL, VH, CL and CH1 domains; (ii) F(ab′)2 fragments, a bivalent fragment comprising two Fab fragments linked by a disulfide bridge at the hinge region; (iii) Fd fragments consisting of the VH and CH1 domains; (iv) Fv fragments consisting of the VL and VH domains of a single arm of an antibody; (v) single-chain Fv (scFv) molecules (see, e.g., Bird et al., (1988) Science 242:423-426; and Huston et al., (1988) Proc. Nat'l. Acad. Sci. USA 85:5879-5883); (vi) dAb fragments (see, e.g., Ward et al., (1989) Nature 341: 544-546); and (vii) minimal recognition units consisting of the amino acid residues that mimic the hypervariable region of an antibody (e.g., an isolated complementarity determining region (CDR)). Other forms of single chain antibodies, such as diabodies are also encompassed. The term antigen binding portion of an antibody includes a “single chain Fab fragment” otherwise known as an “scFab,” comprising an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids.

Bias: In the context of the present disclosure, the term “bias” (as in “biased binding”) refers to skewed or uneven affinity towards or against a subset of antigens to which an antibody is capable of specifically binding. For example, an antibody is said to have bias when the affinity for one antigen complex and the affinity for another antigen complex are not equivalent. Context-independent antibodies according to the present disclosure have equivalent affinities towards such antigen complexes (i.e., unbiased or uniform).

Binding region: As used herein, a “binding region” is a portion of an antigen that, when bound to an antibody or a fragment thereof, can form an interface of the antibody-antigen interaction. Upon antibody binding, a binding region becomes protected from surface exposure, which can be detected by suitable techniques, such as HDX-MS. Antibody-antigen interaction may be mediated via multiple (e.g., two or more) binding regions. A binding region can comprise an antigenic determinant, or epitope.

Biolayer Interferometry (BLI): BLI is a label-free technology for optically measuring biomolecular interactions, e.g., between a ligand immobilized on the biosensor tip surface and an analyte in solution. BLI provides the ability to monitor binding specificity, rates of association and dissociation, or concentration, with precision and accuracy. BLI platform instruments are commercially available, for example, from ForteBio and are commonly referred to as the Octet@System.

Cancer The term “cancer” as used herein refers to the physiological condition in multicellular eukaryotes that is typically characterized by unregulated cell proliferation and malignancy. The term broadly encompasses, solid and liquid malignancies, including tumors, blood cancers (e.g., leukemias, lymphomas and myelomas), as well as myelofibrosis.

Cell-associated proTGFβ1: The term refers to TGFβ1 or its signaling complex (e.g., pro/latent TGFβ1) that is membrane-bound (e.g., tethered to cell surface). Typically, such cell is an immune cell. TGFβ1 that is presented by GARP or LRRC33 is a cell-associated TGFβ1. GARP and LRRC33 are transmembrane presenting molecules that are expressed on cell surface of certain cells. GARP-proTGFβ1 and LRRC33− may be collectively referred to as “cell-associated” (or “cell-surface”) proTGFβ1 complexes, that mediate cell proTGFβ1-associated (e.g., immune cell-associated) TGFβ1 activation/signaling. The term also includes recombinant, purified GARP-proTGFβ1 and LRRC33-proTGFβ1 complexes in solution (e.g., in vitro assays) which are not physically attached to cell membranes. Average KD values of an antibody (or its fragment) to a GARP-proTGFβ1 complex and an LRRC33-proTGFβ1 complex may be calculated to collectively represent affinities for cell-associated (e.g., immune cell-associated) proTGFβ1 complexes. See, for example, Table 5, column (G). Human counterpart of a presenting molecule or presenting molecule complex may be indicated by an “h” preceding the protein or protein complex, e.g., “hGARP,” “hGARP-proTGFβ1,” hLRRC33” and “hLRRC33-proTGFβ1.” In addition to blocking release of active TGFβ1 growth factor from cell-tethered complexes, cell-associated proTGFβ1 may be a target for internalization (e.g., endocytosis) and/or cell killing such as ADCC, ADCP, or ADC-mediated depletion of the target cells expressing such cell surface complexes.

Checkpoint inhibitor: In the context of this disclosure, checkpoint inhibitors refer to immune checkpoint inhibitors and carries the meaning as understood in the art. A “checkpoint inhibitor therapy” or “checkpoint blockade therapy” is one that targets a checkpoint molecule to partially or fully alter its function. Typically, a checkpoint is a receptor molecule on a T cell or NK cell, or a corresponding cell surface ligand on an antigen-presenting cell (APC) or tumor cell. Without being bound by theory, immune checkpoints are activated in immune cells to prevent inflammatory immunity developing against the “self”. Therefore, changing the balance of the immune system via checkpoint inhibition may allow it to be fully activated to detect and eliminate the cancer. The best known inhibitory receptors implicated in control of the immune response are cytotoxic T-lymphocyte antigen-4 (CTLA-4), programmed cell death protein 1 (PD-1), programmed cell death receptor ligand 1 (PD-L1), T-cell immunoglobulin domain and mucin domain-3 (TIM3), lymphocyte-activation gene 3 (LAG3), killer cell immunoglobulin-like receptor (KIR), glucocorticoid-induced tumor necrosis factor receptor (GITR) and V-domain immunoglobulin (Ig)-containing suppressor of T-cell activation (VISTA). Non-limiting examples of checkpoint inhibitors include: Nivolumab, Pembrolizumab, cemiplimab, BMS-936559, Atezolizumab, Avelumab, Durvalumab, Ipilimumab, Tremelimumab, IMP-321 (Eftilagimod alpha or ImmuFact®), BMS-986016 (Relatlimab), budigalimab (ABBV-181), and Lirilumab. Keytruda® is one example of anti-PD-1 antibodies, while Opdivo® is one example of an anti-PD-L1 antibody. Therapies that employ one or more of immune checkpoint inhibitors may be referred to as checkpoint blockade therapy (CBT) or checkpoint inhibitor therapy (CPI).

Clinical benefit: As used herein, the term “clinical benefits” is intended to include both efficacy and safety of a therapy. Thus, therapeutic treatment that achieves a desirable clinical benefit is both efficacious (e.g., achieves therapeutically beneficial effects) and safe (e.g., with tolerable or acceptable levels of toxicities or adverse events).

Combination therapy: “Combination therapy” refers to treatment regimens for a clinical indication that comprise two or more therapeutic agents. Thus, the term refers to a therapeutic regimen in which a first therapy comprising a first composition (e.g., active ingredient) is administered in conjunction with at least a second therapy comprising a second composition (active ingredient) to a patient, intended to treat the same or overlapping disease or clinical condition. The term may further encompass a therapeutic regimen in which a first therapy comprising a first composition (e.g., active ingredient) is administered in conjunction with a second therapy comprising a second composition (e.g., active ingredient such as a checkpoint inhibitor), a third therapy comprising a third composition (e.g., active ingredient such as a chemotherapy), or more (e.g., additional distinct active ingredients). The first, second, and (optionally additional) compositions may act on the same cellular target, or discrete cellular targets. The phrase “in conjunction with,” in the context of combination therapies, means that therapeutic effects of a first therapy overlaps temporally and/or spatially with therapeutic effects of a second and additional therapy in the subject receiving the combination therapy. The first, second, and/or additional compositions may be administered concurrently (e.g., simultaneously), separately, or sequentially. Thus, the combination therapies may be formulated as a single formulation for concurrent administration, or as separate formulations, for sequential, concurrent, or simultaneous administration of the therapies. When a subject who has been treated with a first therapy to treat a disease is administered with a second and additional therapies to treat the same disease, the second and additional therapies may be referred to as an add-on therapy or adjunct therapy.

Combinatory or combinatorial epitope: A combinatorial epitope is an epitope that is recognized and bound by a combinatorial antibody at a site (i.e., antigenic determinant) formed by non-contiguous portions of a component or components of an antigen, which, in a three-dimensional structure, come together in close proximity to form the epitope. Thus, antibodies of the disclosure may bind an epitope formed by two or more components (e.g., portions or segments) of a pro/latent TGFβ1 complex. A combinatory epitope may comprise amino acid residue(s) from a first component of the complex, and amino acid residue(s) from a second component of the complex, and so on. Each component may be of a single protein or of two or more proteins of an antigenic complex. A combinatory epitope is formed with structural contributions from two or more components (e.g., portions or segments, such as amino acid residues) of an antigen or antigen complex.

Compete or cross-compete; cross-block: The term “compete” when used in the context of antigen binding proteins (e.g., an antibody or antigen binding portion thereof) that compete for the same epitope means competition between antigen binding proteins as determined by an assay in which the antigen binding protein being tested prevents or inhibits (e.g., reduces) specific binding of a reference antigen binding protein to a common antigen (e.g., TGFβ1 or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay; solid phase direct biotin-avidin EIA; solid phase direct labeled assay, and solid phase direct labeled sandwich assay. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more when the competing antibody is present in excess. In some embodiments, an SPR (e.g., Biacore) assay is used to determine competition. In some embodiments, a BLI (e.g., Octet®) assay is used to determine competition

In some embodiments, a first antibody or antigen-binding portion thereof and a second antibody or antigen-binding portion thereof “cross-block” with each other with respect to the same antigen, for example, as assayed by Biolayer Interferometry (such as Octet®) or by surface plasmon resonance (such as Biacore System), using standard test conditions, e.g., according to the manufacturer's instructions (e.g., binding assayed at room temperature, ˜20-25° C.). In some embodiments, the first antibody or fragment thereof and the second antibody or fragment thereof may have the same epitope. In other embodiments, the first antibody or fragment thereof and the second antibody or fragment thereof may have non-identical but overlapping epitopes. In yet further embodiments, the first antibody or fragment thereof and the second antibody or fragment thereof may have separate (different) epitopes which are in close proximity in a three-dimensional space, such that antibody binding is cross-blocked via steric hindrance. “Cross-block” means that binding of the first antibody to an antigen prevents binding of the second antibody to the same antigen, and similarly, binding of the second antibody to an antigen prevents binding of the first antibody to the same antigen.

Antibody binning (sometimes referred to as epitope binning or epitope mapping) may be carried out to characterize and sort a set (e.g., “a library”) of monoclonal antibodies made against a target protein or protein complex (i.e., antigen). Such antibodies against the same target are tested against all other antibodies in the library in a pairwise fashion to evaluate if antibodies block one another's binding to the antigen. Closely related binning profiles indicate that the antibodies have the same or closely related (e.g., overlapping) epitope and are “binned” together. Binning provides useful structure-function profiles of antibodies that share similar binding regions within the same antigen because biological activities (e.g., intervention; potency) effectuated by binding of an antibody to its target is likely to be carried over to another antibody in the same bin. Thus, among antibodies within the same epitope bin, those with higher affinities (lower KD) typically have greater potency.

In some embodiments, an antibody that binds the same epitope as Ab6 binds a proTGFβ1 complex such that the epitope of the antibody includes one or more amino acid residues of Region 1, Region 2 and Region 3, identified as the binding region of Ab6.

Complementary determining region: As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al., (1987; 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al., (1989) Nature 342: 877-883) found that certain sub-portions within Kabat CDRs adopt nearly identical peptide backbone conformations, despite having great diversity at the level of amino acid sequence. These sub-portions were designated as L1, L2 and L3 or H1, H2 and H3, or L-CDR1, L-CDR2 and L-CDR3 or H-CDR1, H-CDR2 and H-CDR3, where the “L” and the “H” designate the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding (see, for example: Lu X et al., MAbs. 2019 January; 11(1):45-57). The methods used herein may utilize CDRs defined according to any of these systems, although certain embodiments use Kabat or Chothia defined CDRs.

Conformational epitope: A conformational epitope is an epitope that is recognized and bound by a conformational antibody in a three-dimensional conformation, but not in an unfolded peptide of the same amino acid sequence. A conformational epitope may be referred to as a conformation-specific epitope, conformation-dependent epitope, or conformation-sensitive epitope. A corresponding antibody or fragment thereof that specifically binds such an epitope may be referred to as conformation-specific antibody, conformation-selective antibody, or conformation-dependent antibody. Binding of an antigen to a conformational epitope depends on the three-dimensional structure (conformation) of the antigen or antigen complex.

Constant region: An immunoglobulin constant domain refers to a heavy or light chain constant domain. Human IgG heavy chain and light chain constant domain amino acid sequences are known in the art.

Context-biased: As used herein, “context-biased antibodies” refer to a type of conformational antibodies that binds an antigen with differential affinities when the antigen is associated with (i.e., bound to or attached to) an interacting protein or a fragment thereof. Thus, a context-biased antibody that specifically binds an epitope within proTGFβ1 may bind LTBP1-proTGFβ1, LTBP3-proTGFβ1, GARP-proTGFβ1 and LRRC33-proTGFβ1 with different affinities. For example, an antibody is said to be “matrix-biased” if it has higher affinities for matrix-associated proTGFβ1 complexes (e.g., LTBP1-proTGFβ1 and LTBP3-proTGFβ1) than for cell-associated proTGFβ1 complexes (e.g., GARP-proTGFβ1 and LRRC33-proTGFβ1). Relative affinities of [matrix-associated complexes]: [cell-associated complexes] may be obtained by taking average K_{D} values of the former, taking average K_{D} values of the latter, and calculating the ratio of the two, as exemplified herein.

Context-independent: According to the present disclosure, “a context-independent antibody” that binds proTGFβ1 has equivalent affinities across the four known presenting molecule-proTGFβ1 complexes, namely, LTBP1-proTGFβ1, LTBP3-proTGFβ1, GARP-proTGFβ1 and LRRC33-proTGFβ1. Context-independent antibodies disclosed in the present application may also be characterized as unbiased. Typically, context-independent antibodies show equivalent (i.e., no more than five-fold bias in) affinities, such that relative ratios of measured KD values between matrix-associated complexes and cell-associated complexes are no greater than 5 as measured by a suitable in vitro binding assay, such as surface plasmon resonance, Biolayer Interferometry (BLI), and/or solution equilibrium titration (e.g., MSD-SET). In a preferred embodiment, surface plasmon resonance is used.

ECM-associated TGFβ1/proTGFβ1: The term refers to TGFβ1 or its signaling complex (e.g., pro/latent TGFβ1) that is a component of (e.g., deposited into) the extracellular matrix. TGFβ1 that is presented by LTBP1 or LTBP3 is an ECM-associated TGFβ1, namely, LTBP1-proTGFβ1 and LTBP3-proTGFβ1, respectively. LTBPs are critical for correct deposition and subsequent bioavailability of TGFβ in the ECM, where fibrillin (Fbn) and fibronectin (FN) are believed to be the main matrix proteins responsible for the association of LTBPs with the ECM. Such matrix-associated latent complexes are enriched in connective tissues, as well as certain disease-associated tissues, such as tumor stroma and fibrotic tissues. Human counterpart of a presenting molecule or presenting molecule complex may be indicated by an “h” preceding the protein or protein complex, e.g., “hLTBP1,” “hLTBP1-proTGFβ1,” hLTBP3” and “hLTBP3-proTGFβ1.”

Effective amount: The terms “effective” and “therapeutically effective” refer to the ability or an amount to sufficiently produce a detectable change in a parameter of a disease, e.g., a slowing, pausing, reversing, diminution, or amelioration in a symptom or downstream effect of the disease. The term encompasses but does not require the use of an amount that completely cures a disease. An “effective amount” (or therapeutically effective amount, or therapeutic dose) may be a dosage or dosing regimen that achieves a statistically significant clinical benefit (e.g., efficacy) in a patient population. For example, Ab6 has been shown to be efficacious at doses as low as 3 mg/kg and as high as 30 mg/kg in preclinical models. The term “minimum effective dose” or “minimum effective amount” refers to the lowest amount, dosage, or dosing regimen that achieves a detectable change in a parameter of a disease, e.g., a statistically significant clinical benefit. References herein to a dose of an agent (e.g., a dose of a TGFβ1 inhibitor) may be a therapeutically effective dose, as described herein. In a clinical setting, such as human clinical trials, the term “pharmacological active dose (PAD)” may be used to refer to effective dosage. Effective amounts may be expressed in terms of doses being administered or in terms of exposure levels achieved as a result of administration (e.g., serum concentrations).

Effective tumor control: The term “effective tumor control” may be used to refer to a degree of tumor regression achieved in response to treatment, where, for example, the tumor is regressed by a defined fraction (such as <25%) of an endpoint tumor volume. For instance, in a particular model, if the endpoint tumor volume is set at 2,000 mm³, effective tumor control is achieved if the tumor is reduced to less than 500 mm³ assuming the threshold of <25%. Therefore, effective tumor control encompasses complete regression. Clinically, effective tumor control can be measured by objective response, which includes partial response (PR) and complete response (CR) as determined by art-recognized criteria, such as RECIST v1.1 and corresponding iRECIST (iRECIST v1.1). In some embodiments, effective tumor control in clinical settings also includes stable disease, where tumors that are typically expected to grow at certain rates are prevented from such growth by the treatment, even though shrinkage is not achieved.

Effector T cells: Effector T cells, as used herein, are T lymphocytes that actively respond immediately to a stimulus, such as co-stimulation and include, but are not limited to, CD4+ T cells (also referred to as T helper or Th cells) and CD8+ T cells (also referred to as cytotoxic T cells). Th cells assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including Th1, Th2, Th3, Th17, Th9, or TFh, which secrete different cytokines to facilitate different types of immune responses. Signaling from the APC directs T cells into particular subtypes. Cytotoxic (Killer). Cytotoxic T cells (TC cells, CTLs, T-killer cells, killer T cells), on the other hand, destroy virus-infected cells and cancer cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Cytotoxic effector cell (e.g., CD8+ cells) markers include, e.g., perforin and granzyme B.

Endpoint: In studies aimed to assess effectiveness (e.g., clinical benefit or improvements) of a therapy, such as in clinical trials for a cancer therapy, endpoints represent the measures of predetermined parameters indicative of treatment effects. In oncology, suitable endpoints may include overall survival, disease-free survival (DFS), event-free survival (EFS), progression-free survival (PFS), objective response rate (ORR), complete response (CR), partial response (PR), time to progression (TTP), as well as patient-reported outcomes (e.g., symptom assessment) and biomarker assessment such as blood or body fluid-based assessments.

Epithelial hyperplasia: The term “epithelial hyperplasia” refers to an increase in tissue growth resulting from proliferation of epithelial cells. As used herein, epithelial hyperplasia refers to the undesired toxicity resulting from TGFβ inhibition which may include, but is not limited to, abnormal growth of epithelial cells in the oral cavity, esophagus, breast, and ovary.

Epitope: The term “epitope” may be also referred to as an antigenic determinant, is a molecular determinant (e.g., polypeptide determinant) that can be specifically bound by a binding agent, immunoglobulin, or T-cell receptor. Epitope determinants include chemically active surface groupings of molecules, such as amino acids, sugar side chains, phosphoryl, or sulfonyl, and, in certain embodiments, may have specific three-dimensional structural characteristics, and/or specific charge characteristics. An epitope recognized by an antibody or an antigen-binding fragment of an antibody is a structural element of an antigen that interacts with CDRs (e.g., the complementary site) of the antibody or the fragment. An epitope may be formed by contributions from several amino acid residues, which interact with the CDRs of the antibody to produce specificity. An antigenic fragment can contain more than one epitope. In certain embodiments, an antibody may specifically bind an antigen when it recognizes its target antigen in a complex mixture of proteins and/or macromolecules. For example, antibodies are said to “bind to the same epitope” if the antibodies cross-compete (one prevents the binding or modulating effect of the other).

Extended Latency Lasso: The term “Extended Latency Lasso” as used herein refers to a portion of the prodomain that comprises Latency Lasso and Alpha-2 Helix, e.g., LASPPSQGEVPPGPLPEAVLALYNSTR (SEQ ID NO: 127). In some embodiments, Extended Latency Lasso further comprises a portion of Alpha-1 Helix, e.g., LVKRKRIEA (SEQ ID NO: 132) or a portion thereof.

Fibrosis: The term “fibrosis” or “fibrotic condition/disorder” refers to the process or manifestation characterized by the pathological accumulation of extracellular matrix (ECM) components, such as collagens, within a tissue or organ.

Finger-1 (of TGFβ1 Growth Factor): As used herein, “Finger-1” is a domain within the TGFβ1 growth factor domain. In its unmutated form, Finger-1 of human proTGFβ1 contains the following amino acid sequence: CVRQLYIDFRKDLGWKWIHEPKGYHANFC (SEQ ID NO: 124). In the 3D structure, the Finger-1 domain comes in close proximity to Latency Lasso.

Finger-2 (of TGFβ1 Growth Factor): As used herein, “Finger-2” is a domain within the TGFβ1 growth factor domain. In its unmutated form, Finger-2 of human proTGFβ1 contains the following amino acid sequence: CVPQALEPLPIVYYVGRKPKVEQLSNMIVRSCKCS (SEQ ID NO: 125). Finger-2 includes the “binding region 6”, which spatially lies in close proximity to Latency Lasso.

GARP-proTGFβ1 complex: As used herein, the term “GARP-TGFβ1 complex” refers to a protein complex comprising a pro-protein form or latent form of a transforming growth factor-β1 (TGFβ1) protein and a glycoprotein-A repetitions predominant protein (GARP) or fragment or variant thereof. In some embodiments, a pro-protein form or latent form of TGFβ1 protein may be referred to as “pro/latent TGFβ1 protein”. In some embodiments, a GARP-TGFβ1 complex comprises GARP covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a GARP-TGFβ1 complex comprises GARP non-covalently linked with pro/latent TGFβ1. In some embodiments, a GARP-TGFβ1 complex is a naturally-occurring complex, for example a GARP-TGFβ1 complex in a cell. The term “hGARP” denotes human GARP.

High-affinity: As used herein, the term “high-affinity” as in “a high-affinity proTGFβ1 antibody” refers to in vitro binding activities having a K_{D} value of ≤5 nM, more preferably ≤1 nM. Thus, a high-affinity, context-independent proTGFβ1 antibody encompassed by the disclosure herein has a K_{D} value of ≤5 nM, more preferably ≤1 nM, towards each of the following antigen complexes: LTBP1-proTGFβ1, LTBP3-proTGFβ1, GARP-proTGFβ1 and LRRC33-proTGFβ1.

Human antibody: The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Humanized antibody: The term “humanized antibody” refers to antibodies, which comprise heavy and light chain variable region sequences from a non-human species (e.g., a mouse) but in which at least a portion of the VH and/or VL sequence has been altered to be more “human-like,” i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody, or a variant, derivative, analog or fragment thereof, which immunospecifically binds to an antigen of interest and which comprises an FR region having substantially the amino acid sequence of a human antibody and a CDR region having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the FR regions are those of a human immunoglobulin consensus sequence. In an embodiment a humanized antibody also comprises at least a portion of an immunoglobulin Fc region, typically that of a human immunoglobulin. In some embodiments a humanized antibody contains the light chain as well as at least the variable domain of a heavy chain. The antibody also may include the CH1, hinge, CH2, CH3, and CH4 regions of the heavy chain. In some embodiments a humanized antibody only contains a humanized light chain. In some embodiments a humanized antibody only contains a humanized heavy chain. In specific embodiments a humanized antibody only contains a humanized variable domain of a light chain and/or humanized heavy chain.

Immune-excluded or immuno-excluded tumor As used herein, tumors characterized as “immune excluded” are devoid of or substantially devoid of intratumoral anti-tumor lymphocytes. For example, tumors with poorly infiltrated T cells may have T cells that surround the tumor, e.g., the external perimeters of a tumor mass and/or near the vicinity of vasculatures (“perivascular”) of a tumor, which nevertheless fail to effectively swarm into the tumor to exert cytotoxic function against cancer cells. In other situations, tumors fail to provoke a strong immune response (so-called “immune desert” tumors) such that few T cells are present near and in the tumor environment. In contrast to immune-excluded tumors, tumors that are infiltrated with anti-tumor lymphocytes are sometimes characterized as “hot” or “inflamed” tumors; such tumors tend to be more responsive to and therefore are the target of immune checkpoint blockade therapies (“CBTs”). Typically, however, only a fraction of patients responds to a CBT due to immune exclusion that renders the tumor resistant to the CBT.

Immune safety (assessment): As used herein, the term refers to safety assessment related to immune responses (immune activation), Acceptable immune safety criteria include no significant cytokine release as determined by in vitro or in vivo cytokine release testing (e.g., assays); and no significant platelet aggregation, activation as determined with human platelets. Statistical significance in these studies may be determined against a suitable control as reference. For example, for a test molecule which is a human monoclonal antibody, a suitable control may be an immunoglobulin of the same subtype, e.g., an antibody of the same subtype known to have a good safety profile in a human.

Immunosuppression, immune suppression, immunosuppressive: The terms refer to the ability to suppress immune cells, such as T cells, NK cells and B cells. The gold standard for evaluating immunosuppressive function is the inhibition of T cell activity, which may include antigen-specific suppression and non-specific suppression. Regulatory T cells (Tregs) and MDSCs may be considered immunosuppressive cells. M2-polarized macrophages (e.g., disease-localized macrophages such as TAMs and FAMs) may also be characterized as immunosuppressive.

Immunological memory: Immunological memory refers to the ability of the immune system to quickly and specifically recognize an antigen that the body has previously encountered and initiate a corresponding immune response. Generally, these are secondary, tertiary, and other subsequent immune responses to the same antigen. Immunological memory is responsible for the adaptive component of the immune system, special T and B cells—the so-called memory T and B cells. Antigen-naïve T cells expand and differentiate into memory and effector T cells after they encounter their cognate antigen within the context of an MHC molecule on the surface of a professional antigen presenting cell (e.g., a dendritic cell). The single unifying theme for all memory T cell subtypes is that they are long-lived and can quickly expand to large numbers of effector T cells upon re-exposure to their cognate antigen. By this mechanism they provide the immune system with “memory” against previously encountered pathogens. Memory T cells may be either CD4+ or CD8+ and usually express CD45RO. In a preclinical setting, immunological memory may be tested in a tumor rechallenge paradigm.

Inhibit or inhibition of: The term “inhibit” or “inhibition of,” as used herein, means to reduce by a measurable amount, and can include but does not require complete prevention or inhibition.

Isoform-non-specific: The term “isoform non-specific” refers to an agent's ability to bind to more than one structurally related isoforms. An isoform-non-specific TGFβ inhibitor exerts its inhibitory activity toward more than one isoform of TGFβ, such as TGFβ1/3, TGFβ1/2, TGFβ2/3, and TGFβ1/2/3.

Isoform-specific: The term “isoform specificity” refers to an agent's ability to discriminate one isoform over other structurally related isoforms (i.e., isoform selectivity). An isoform-specific TGFβ inhibitor exerts its inhibitory activity towards one isoform of TGFβ but not the other isoforms of TGFβ at a given concentration. For example, an isoform-specific TGFβ1 antibody selectively binds TGFβ1. A TGFβ1-specific inhibitor (antibody) preferentially targets (binds thereby inhibits) the TGFβ1 isoform over TGFβ2 or TGFβ3 with substantially greater affinity. For example, the selectivity in this context may refer to at least a 10-fold, 100-fold, 500-fold, 1000-fold, or greater difference in respective affinities as measured by an in vitro binding assay such as BLI (Octet®) or preferably SPR (Biacore®). In some embodiments, the selectivity is such that the inhibitor when used at a dosage effective to inhibit TGFβ1 in vivo does not inhibit TGFβ2 and TGFβ3. For such an inhibitor to be useful as a therapeutic, dosage to achieve desirable effects (e.g., therapeutically effective amounts) must fall within the window within which the inhibitor can effectively inhibit the TGFβ1 isoform without inhibiting TGFβ2 or TGFβ3. In some embodiments, a TGFβ31-selective inhibitor is a pharmacological agent that interferes with the function or activities of TGFβ1, but not of TGFβ2 and/or TGFβ3, irrespective of the mechanism of action.

Isolated: An “isolated” antibody as used herein, refers to an antibody that is substantially free of other antibodies having different antigenic specificities. In some embodiments, an isolated antibody is substantially free of other unintended cellular material and/or chemicals.

Large Latent Complex: The term “large latent complex” (“LLC”) in the context of the present disclosure refers to a complex comprised of a proTGFβ1 dimer bound to so-called a presenting molecule. Thus, a large latent complex is a presenting molecule-proTGFβ1 complex, such as LTBP1-proTGFβ1, LTBP3-proTGFβ1, GARP-proTGFβ1 and LRRC33-proTGFβ1. Such complexes may be formed in vitro using recombinant, purified components capable of forming the complex. For screening purposes, presenting molecules used for forming such LLCs need not be full length polypeptides; however, the portion of the protein capable of forming disulfide bonds with the proTGFβ1 dimer complex via the cysteine residues near its N-terminal regions is typically required.

Latency associated peptide (LAP): LAP is so-called the “prodomain” of proTGFβ1. As described in more detail herein, LAP is comprised of the “Straight Jacket” domain and the “Arm” domain. Straight Jacket itself is further divided into the Alpha-1 Helix and Latency Lasso domains.

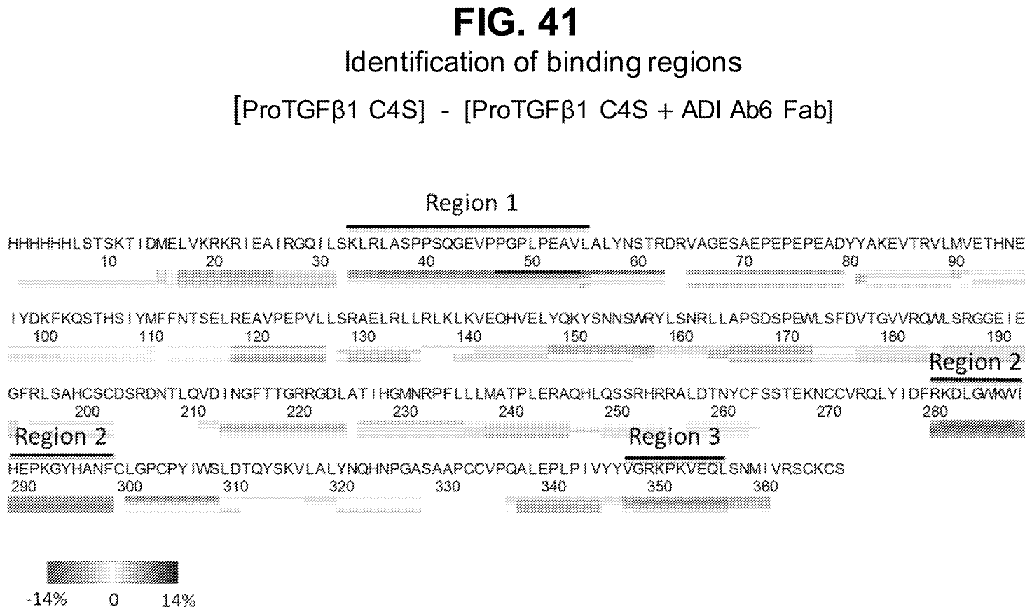

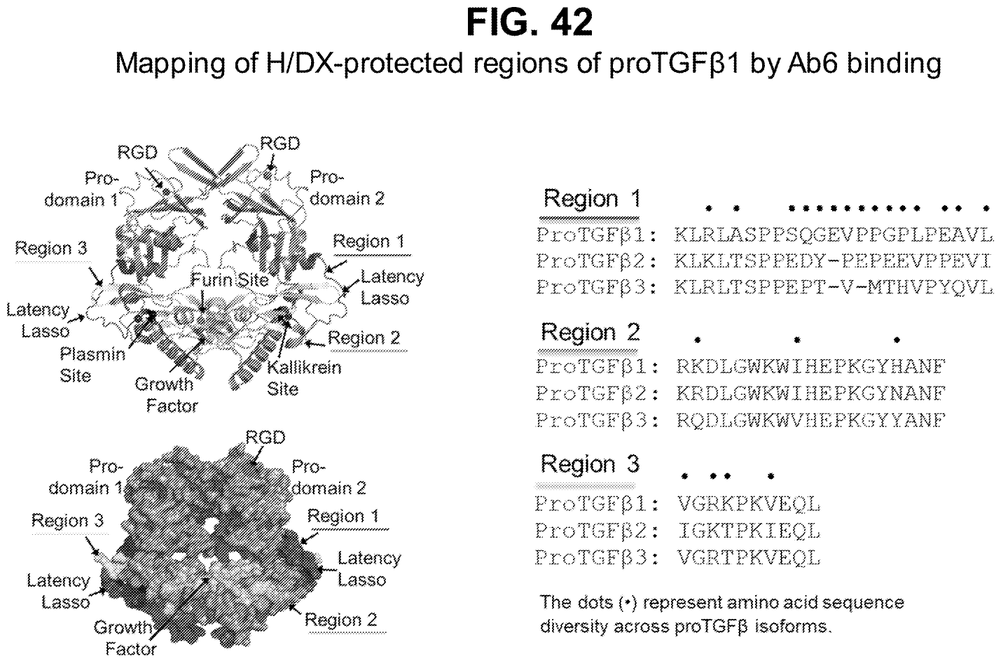

Latency Lasso: As used herein, “Latency Lasso,” sometimes also referred to as Latency Loop, is a domain flanked by Alpha-1 Helix and the Arm within the prodomain of proTGFb1. In its unmutated form, Latency Lasso of human proTGFβ1 comprises the amino acid sequence: LASPPSQGEVPPGPL (SEQ ID NO: 126) which is spanned by Region 1 identified in FIG. 41. As used herein, the term Extended Latency Lasso region” refers to the Latency Lasso together with its immediate C-terminal motif referred to as Alpha-2 Helix (α2-Helix) of the prodomain. The proline residue that is at the C-terminus of the Latency Lasso provides the perpendicular “turn” like an “elbow” that connects the lasso loop to the α2-Helix. Certain high affinity TGFβ1 activation inhibitors bind at least in part to Latency Lasso or a portion thereof to confer the inhibitory potency (e.g., the ability to block activation), wherein optionally the portion of the Latency Lasso is ASPPSQGEVPPGPL (SEQ ID NO: 170). In some embodiments, the antibodies of the present disclosure bind a proTGFβ1 complex at ASPPSQGEVPPGPL (SEQ ID NO: 170) or a portion thereof. Certain high affinity TGFβ1 activation inhibitors bind at least in part to Extended Latency Lasso or a portion thereof to confer the inhibitory potency (e.g., the ability to block activation), wherein optionally the portion of the Extended Latency Lasso is KLRLASPPSQGEVPPGPLPEAVL (SEQ ID NO: 142).

Localized: In the context of the present disclosure, the term “localized” (as in “localized tumor”, “disease-localized” etc.) refers to anatomically isolated or isolatable abnormalities, such as solid malignancies, as opposed to systemic disease. Certain leukemia, for example, may have both a localized component (for instance the bone marrow) and a systemic component (for instance circulating blood cells) to the disease.

LRRC33-proTGFβ1 complex: As used herein, the term “LRRC33-TGFβ1 complex” refers to a complex between a pro-protein form or latent form of transforming growth factor-31 (TGFβ1) protein and a Leucine-Rich Repeat-Containing Protein 33 (LRRC33; also known as Negative Regulator of Reactive Oxygen Species or NRROS) or fragment or variant thereof. In some embodiments, a LRRC33-TGFβ1 complex comprises LRRC33 covalently linked with pro/latent TGFβ1 via one or more disulfide bonds. In nature, such covalent bonds are formed with cysteine residues present near the N-terminus (e.g., amino acid position 4) of a proTGFβ1 dimer complex. In other embodiments, a LRRC33-TGFβ1 complex comprises LRRC33 non-covalently linked with pro/latent TGFβ1. In some embodiments, a LRRC33-TGFβ1 complex is a naturally-occurring complex, for example a LRRC33-TGFβ1 complex in a cell. The term “hLRRC33” denotes human LRRC33. In vivo, LRRC33 and LRRC33-containing complexes on cell surface may be internalized. LRRC33 is expressed on a subset of myeloid cells, including M2-polarized macrophages (such as TAMs) and MDSCs. MDSCs that express LRRC33 on cell surface include tumor-associated MDSCs and circulatory MDSCs. LRRC33-expressing tumor-associated MDSCs may include gMDSCs. LRRC33-expressing MDSCs in circulation may include g-MDSCs.

LTBP1-proTGFβ1 complex: As used herein, the term “LTBP1-TGFβ1 complex” refers to a protein complex comprising a pro-protein form or latent form of transforming growth factor-31 (TGFβ1) protein and a latent TGF-beta binding protein 1 (LTBP1) or fragment or variant thereof. In some embodiments, a LTBP1-TGFβ1 complex comprises LTBP1 covalently linked with pro/latentTGFI31 via one or more disulfide bonds. In nature, such covalent bonds are formed with cysteine residues present near the N-terminus (e.g., amino acid position 4) of a proTGFβ1 dimer complex. In other embodiments, a LTBP1-TGFβ1 complex comprises LTBP1 non-covalently linked with pro/latent TGFβ1. In some embodiments, a LTBP1-TGFβ1 complex is a naturally-occurring complex, for example a LTBP1-TGFβ1 complex in a cell. The term “hLTBP1” denotes human LTBP1.

LTBP3-proTGFβ1 complex: As used herein, the term “LTBP3-TGFβ1 complex” refers to a protein complex comprising a pro-protein form or latent form of transforming growth factor-31 (TGFβ1) protein and a latent TGF-beta binding protein 3 (LTBP3) or fragment or variant thereof. In some embodiments, a LTBP3-TGFβ1 complex comprises LTBP3 covalently linked with pro/latentTGFI31 via one or more disulfide bonds. In nature, such covalent bonds are formed with cysteine residues present near the N-terminus (e.g., amino acid position 4) of a proTGFβ1 dimer complex. In other embodiments, a LTBP3-TGFβ1 complex comprises LTBP1 non-covalently linked with pro/latent TGFβ1. In some embodiments, a LTBP3-TGFβ1 complex is a naturally-occurring complex, for example a LTBP3-TGFβ1 complex in a cell. The term “hLTBP3” denotes human LTBP3.

M2 or M2-like macrophage: M2 macrophages represent a subset of activated or polarized macrophages and include disease-associated macrophages in both fibrotic and tumor microenvironments. Cell-surface markers for M2-polarized macrophages typically include CD206 and CD163 (i.e., CD206+/CD163+). M2-polarized macrophages may also express cell-surface LRRC33. Activation of M2 macrophages is promoted mainly by IL-4, IL-13, IL-10 and TGFβ; they secrete the same cytokines that activate them (IL-4, IL-13, IL-10 and TGFβ). These cells have high phagocytic capacity and produce ECM components, angiogenic and chemotactic factors. The release of TGFβ by macrophages may perpetuate the myofibroblast activation, EMT and EndMT induction in the disease tissues, such as fibrotic tissue and tumor stroma. For example, M2 macrophages play a role in TGFβ-driven lung fibrosis and are also enriched in a number of tumors.

Matrix-associated proTGFβ1: LTBP1 and LTBP3 are presenting molecules that are components of the extracellular matrix (ECM). LTBP1-proTGFβ1 and LTBP3-proTGFβ1 may be collectively referred to as “ECM-associated” (or “matrix-associated”) proTGFβ1 complexes, that mediate ECM-associated TGFβ1 activation/signaling. The term also includes recombinant, purified LTBP1-proTGFβ1 and LTBP3-proTGFβ1 complexes in solution (e.g., in vitro assays) which are not physically attached to a matrix or substrate.

Maximally tolerated dose (MTD): The term MTD generally refers to, in the context of safety/toxicology considerations, the highest amount of a test article (such as a TGFβ1 inhibitor) evaluated with no-observed-adverse-effect level (NOAEL). For example, the NOAEL for Ab6 in rats was the highest dose evaluated (100 mg/kg), suggesting that the MTD for Ab6 is >100 mg/kg, based on a four-week toxicology study. The NOAEL for Ab6 in non-human primates was the highest dose evaluated (300 mg/kg), suggesting that the MTD for Ab6 in the non-human primates is >300 mg/kg, based on a four-week toxicology study.

Meso-Scale Discovery: “Meso-Scale Discovery” or “MSD” is a type of immunoassays that employs electrochemiluminescence (ECL) as a detection technique. Typically, high binding carbon electrodes are used to capture proteins (e.g., antibodies). The antibodies can be incubated with particular antigens, which binding can be detected with secondary antibodies that are conjugated to electrochemiluminescent labels. Upon an electrical signal, light intensity can be measured to quantify analytes in the sample.

Myelofibrosis: “Myelofibrosis,” also known as osteomyelofibrosis, is a relatively rare bone marrow proliferative disorder (e.g., cancer), Myelofibrosis is generally characterized by the proliferation of an abnormal clone of hematopoietic stem cells in the bone marrow and other sites results in fibrosis, or the replacement of the marrow with scar tissue. The term myelofibrosis encompasses primary myelofibrosis (PMF), also be referred to as chronic idiopathic myelofibrosis (cIMF) (the terms idiopathic and primary mean that in these cases the disease is of unknown or spontaneous origin), as well as secondary types of myelofibrosis, such as myelofibrosis that develops secondary to polycythemia vera (PV) or essential thrombocythaemia (ET). Myelofibrosis is a form of myeloid metaplasia, which refers to a change in cell type in the blood-forming tissue of the bone marrow, and often the two terms are used synonymously. The terms agnogenic myeloid metaplasia and myelofibrosis with myeloid metaplasia (MMM) are also used to refer to primary myelofibrosis. Myelofibrosis is characterized by mutations that cause upregulation or overactivation of the downstream JAK pathway.

Myeloid cells: In hematopoiesis, myeloid cells are blood cells that arise from a progenitor cell for granulocytes, monocytes, erythrocytes, or platelets (the common myeloid progenitor, that is, CMP or CFU-GEMM), or in a narrower sense also often used, specifically from the lineage of the myeloblast (the myelocytes, monocytes, and their daughter types), as distinguished from lymphoid cells, that is, lymphocytes, which come from common lymphoid progenitor cells that give rise to B cells and T cells. Certain myeloid cell types, their general morphology, typical cell surface markers, and their immune-suppressive ability in both mouse and human, are summarized below. In some embodiments, a human neutrophil can be identified by at least one (e.g., all) of the cell surface markers CD11b⁺, CD14⁻, CD15⁺, and CD66b⁺. In some embodiments, a human neutrophil is LOX-1⁻. In some embodiments, a human neutrophil is HLA-DR^{-/med}. In some embodiments, a classical human monocyte can be identified by at least one (e.g., all) of the cell surface markers CD14⁺ CD15⁻ CD16⁻ HLA-DR⁺. In some embodiments, a classical human monocyte is CD33⁺ and/or CD11b⁺. In some embodiments, a classical human monocyte is CD16⁻. In some embodiments, an intermediate human monocyte can be identified by at least one (e.g., all) of the cell surface markers CD14⁺ D15⁻ CD16S HLA-DR⁺. In some embodiments, a non-classical human monocyte can be identified by at least one (e.g., all) of the cell surface markers CD14⁻ CD15⁻ CD16⁺ HLA-DR⁺. In some embodiments, a human M1 macrophage can be identified by at least one (e.g., all) of the cell surface markers CD15⁻ CD16⁺ CD80⁺ HLA-DR^{+/hugh} CD33⁺. In some embodiments, a human M macrophage is CD66b⁻. In some embodiments, a human Ms macrophage is CD11b⁺. In some embodiments, a human M macrophage is D14. In some embodiments, a human M2 macrophage can be identified by at least one (e.g., all) ofthe cell surface markers CD11b⁺ and CD15⁻. In some embodiments, a human M2 macrophage is CD206⁺. In some embodiments, a human M2 macrophage is CD163⁺. In some embodiments, a human M2 macrophage is HLA-DR⁺. In some embodiments, a human M2 macrophage is CD14⁻. In some embodiments, a human M2 macrophage is CD33⁺. In some embodiments, a human M2 macrophage is CD66b⁻.

| | | | |
|---|---|---|---|
| | | | Immune |
| Myeloid cells | Typical Morphology | Select surface phenotype | suppression |

Mouse

| | | | |
|---|---|---|---|
| Neutrophils | Round shape with a | CD11b⁺ Ly6G^{hi} Ly6C^{lo} | − |
| | segmented nucleus | | |
| Monocytes | Round shape with an | CD11b⁺ Ly6G⁻ Ly6C^{hi} | − |
| | indented nucleus | | |
| Macrophages | Round shape with | CD11b⁺ F4/80^{hi} Ly6G⁻ Ly6C^{lo} | − |
| | pseudopodia | CD80⁺ (M1) | |
| | | F4/80⁺ CD206⁺ CD163⁺ | − |
| | | LRRC33+ (M2) | |
| Dendritic | Dendritic shape | CD11b⁺ CD11c⁺ Ly6G⁻ Ly6C^{-/lo} | − |
| cells | with polypodia | (classical) | |
| | | CD11b⁻ CD11c⁺ Ly6G⁻ Ly6C⁻ | − |
| | | (classical) | |
| | | CD11b⁻ CD11c^{lo} Ly6G⁻ Ly6C⁺ | − |
| | | PDCA-1⁺ (plasmacytoid) | |
| Fibrocytes | Spindle shape | CD11b⁺ Coll⁺ Ly6G⁻ Ly6C⁺ | − |
| G-MDSCs | Round shape with a | CD11b⁺ Ly6G⁺ Ly6C^{lo} | + |
| (PMN-MDSCs) | banded nucleus | LRRC33+ | |
| M-MDSCs | Round shape with an | CD11b⁺ Ly6G⁻ Ly6C^{hi} | + |
| | indented nucleus | LRRC33+ | |

Human

| | | | |
|---|---|---|---|
| Neutrophils | Round shape with a | CD11b⁺ CD14⁻ CD15⁺ CD66b⁺ | − |
| | segmented nucleus | LOX-1⁻ | |
| Monocytes | Round shape with an | CD14⁺ CD15⁻ CD16⁻ HLA-DR⁺ | − |
| | indented nucleus | (classical) | |
| | | CD14⁺ CD15⁻ CD16⁺ HLA-DR⁺ | − |
| | | (intermediate) | |
| | | CD14⁻ CD15⁻ CD16⁺ HLA-DR⁺ | − |
| | | (non-classical) | |
| Macrophages | Round shape with | CD15⁻ CD16⁺ CD80⁺ HLA-DR⁺ | − |
| | pseudopodia | CD33⁺ (M1) | |
| | | CD11b⁺ CD15⁻ CD206⁺ CD163⁺ | +/− |
| | | HLA-DR⁺ (M2) | |
| Dendritic | Dendritic shape | CD14⁻ CD16⁻ CD1C⁺ CD83⁺ | − |
| cells | with polypodia | (classical) | |
| | | CD14⁻ CD16⁻ CD141⁺ CD83⁺ | − |
| | | (classical) | |
| | | CD14⁻ CD16⁻ CD303⁺ CD83⁺ | − |
| | | (plasmacytoid) | |
| Fibrocytes | Spindle shape | CD11b⁺ Coll⁺ CD13⁺ CD34⁺ | − |
| | | CD45RO⁺ HLA-DR⁺ | |
| G-MDSCs | Round shape with an | CD11b⁺ CD33⁺ CD14⁻ CD15⁺ | + |
| (PMN-MDSCs) | annular nucleus | CD66b⁺ LOX-1⁺ HLA-DR^{-/lo} | |
| M-MDSCs | Round shape with an | CD11b⁺ CD33⁺ CD14⁺ CD15⁻ | + |
| | indented nucleus | HLA-DR^{-/lo} | |

Myeloid-derived suppressor cell: Myeloid-derived suppressor cells (MDSCs) are a heterogeneous population of cells generated during various pathologic conditions. MDSCs include at least two categories of cells termed i) “granulocytic” (G-MDSC) or polymorphonuclear (PMN-MDSC), which are phenotypically and morphologically similar to neutrophils; and ii) monocytic (M-MDSC) which are phenotypically and morphologically similar to monocytes. MDSCs are characterized by a distinct set of genomic and biochemical features, and can be distinguished by specific surface molecules. In certain embodiments, suitable cell surface markers for identifying MDSCs may include one or more of CD11b, CD33, CD14, CD15, HLA-DR and CD66b. For example, human G-MDSCs/PMN-MDSCs typically express the cell-surface markers CD11b, CD33, CD15 and CD66b. In some embodiments, human G-MDSCs may express low levels of the CD33 cell surface marker. In some embodiments, human G-MDSCs/PMN-MDSCs may express LOX-1 and/or Arginase. By comparison, human M-MDSCs typically express the cell surface markers CD11 b, CD33 and CD14. Additionally, both human G-MDSCs/PMN-MDSCs and M-MDSCs may also exhibit low levels or undetectable levels of HLA-DR. In some embodiments, human G-MDSCs may be HLA-DR⁻. In certain embodiments, G-MDSCs may be differentiated from M-MDSCs based on the presence or absence of certain cell surface marker (e.g., CD14, CD15, and/or CD66b). In some embodiments, G-MDSCs may be identified by the presence or elevated expression of surface markers CD11 b, CD33, CD15, CD66b, and/or LOX-1, and the absence of CD14, whereas M-MDSCs may be identified by the presence or elevated expression of surface markers CD11 b, CD33, and/or CD14, and the absence of CD15. In some embodiments, M-MDSCs may be CD66b⁻. In addition to such cell-surface markers, MDSCs may be characterized by the ability to suppress immune cells, such as T cells, NK cells and B cells. Immune suppressive functions of MDSCs may include inhibition of antigen-non-specific function and inhibition of antigen-specific function. MDSCs, including tumor-associated MDSCs and MDSCs in circulation, can express cell surface LRRC33 and/or LRRC33-proTGFβ1. In some embodiments, a signal intensity of a cell surface marker may be categorized, or binned, as “low”, “medium”, or “high” based on normalization of signal intensity to reduce background and bleed through signals. In some embodiments, a signal intensity of a cell surface marker may be categorized based on cutoff thresholds provided in Table 38A. In some embodiments, a signal intensity of a cell surface marker may be determined by binary intensity selection. In some embodiments, the binary intensity selection comprises categorizing a signal intensity measured for a particular cell surface marker as “positive” or “negative.” In some embodiments, a signal intensity of a cell surface marker may be categorized based on the cutoff thresholds provided in Table 38B. In some embodiments, signal intensities of a set of surface markers may be determined by sequential application of signal filtering, where the signal intensity threshold for one or more surface markers is determined before the threshold is determined for one or more additional surface markers.

Myofibroblast: Myofibroblasts are cells with certain phenotypes of fibroblasts and smooth muscle cells and generally express vimentin, alpha-smooth muscle actin (α-SMA; human gene ACTA2) and paladin. In many disease conditions involving extracellular matrix dysregulations (such as increased matrix stiffness), normal fibroblast cells become de-differentiated into myofibroblasts in a TGFβ-dependent manner. Aberrant overexpression of TGFβ is common among myofibroblast-driven pathologies. TGFβ is known to promote myofibroblast differentiation, cell proliferation, and matrix production. Myofibroblasts or myofibroblast-like cells within the fibrotic microenvironment may be referred to as fibrosis-associated fibroblasts (or “FAFs”), and myofibroblasts or myofibroblast-like cells within the tumor microenvironment may be referred to as cancer-associated fibroblasts (or “CAFs”).

Pan-TGFβ inhibitor/pan-inhibition of TGFβ: The term “pan-TGFβ inhibitor” refers to any agent that is capable of inhibiting or antagonizing all three isoforms of TGFβ. Such an inhibitor may be a small molecule inhibitor of TGFβ isoforms, such as those known in the art. The term includes pan-TGFβ antibody which refers to any antibody capable of binding to each of TGFβ isoforms, i.e., TGFβ1, TGFβ2, and TGFβ3. In some embodiments, a pan-TGFβ antibody binds and neutralizes activities of all three isoforms, i.e., TGFβ1, TGFβ2, and TGFβ3. The antibody 1D11 (or the human analog fresolimumab (GC1008)) is a well-known example of a pan-TGFβ antibody that neutralizes all three isoforms of TGFβ. Examples of small molecule pan-TGFβ inhibitors include galunisertib (LY2157299 monohydrate), which is an antagonist for the TGFβ receptor I kinase/ALK5 that mediates signaling of all three TGFβ isoforms.

Perivascular (infiltration): The prefix “peri-” means “around” “surrounding” or “near,” hence “perivascular” literally translates to around the blood vessels. As used herein in the context of tumor cell infiltrates, the term “perivascular infiltration” refers to a mode of entry for tumor-infiltrating immune cells (e.g., lymphocytes) via the vasculature of a solid tumor.

Potency: The term “potency” as used herein refers to activity of a drug, such as an inhibitory antibody (or fragment) having inhibitory activity, with respect to concentration or amount of the drug to produce a defined effect. For example, an antibody capable of producing certain effects at a given dosage is more potent than another antibody that requires twice the amount (dosage) to produce equivalent effects. Potency may be measured in cell-based assays, such as TGFβ activation/inhibition assays, whereby the degree of TGFβ activation, such as activation triggered by integrin binding, can be measured in the presence or absence of test article (e.g., inhibitory antibodies) in a cell-based system. Typically, among those capable of binding to the same or overlapping binding regions of an antigen (e.g., cross-blocking antibodies), antibodies with higher affinities (lower K_{D} values) tend to show higher potency than antibodies with lower affinities (greater K_{D} values).

Preclinical model: The term “preclinical model” refers to a cell line or an animal that exhibits certain characteristics of a human disease which is used to study the mechanism of action, efficacy, pharmacology, and toxicology of a drug, procedure, or treatment before it is tested on humans. Typically, cell-based preclinical studies are referred to as “in vitro” studies, whereas animal-based preclinical studies are referred to as “in vivo” studies. For example, in vivo mouse preclinical models encompassed by the current disclosure include the MBT2 bladder cancer model, the Cloudman S91 melanoma model, and the EMT6 breast cancer model.

Predictive biomarker Predictive biomarkers provide information on the probability or likelihood of response to a particular therapy. Typically, a predictive biomarker is measured before and after treatment, and the changes or relative levels of the marker in samples collected from the subject indicates or predicts therapeutic benefit.

Presenting molecule: Presenting molecules in the context of the present disclosure refer to proteins that form covalent bonds with latent pro-proteins (e.g., proTGFβ1) and tether (“present”) the inactive complex to an extracellular niche (such as ECM or immune cell surface) thereby maintaining its latency until an activation event occurs. Known presenting molecules for proTGFβ1 include: LTBP1, LTBP3, GARP and LRRC33, each of which can form a presenting molecule-proTGFβ1 complex (i.e., LLC), namely, LTBP1-proTGFβ1, LTBP3-proTGFβ1, GARP-proTGFβ1 and LRRC33-proTGFβ1, respectively. In nature, LTBP1 and LTBP3 are components of the extracellular matrix (ECM); therefore, LTBP1-proTGFβ1 and LTBP3-proTGFβ1 may be collectively referred to as “ECM-associated” (or “matrix-associated”) proTGFβ1 complexes, that mediate ECM-associated TGFβ1 signaling/activities. GARP and LRRC33, on the other hand, are transmembrane proteins expressed on cell surface of certain cells; therefore, GARP-proTGFβ1 and LRRC33-proTGFβ1 may be collectively referred to as “cell-associated” (or “cell-surface”) proTGFβ1 complexes, that mediate cell-associated (e.g., immune cell-associated) TGFβ1 signaling/activities.

ProTGFβ1: The term “proTGFβ1” as used herein is intended to encompass precursor forms of inactive TGFβ1 complex that comprises a prodomain sequence of TGFβ1 within the complex. Thus, the term can include the pro-, as well as the latent-forms of TGFβ1. The expression “pro/latent TGFβ1” may be used interchangeably. The “pro” form of TGFβ1 exists prior to proteolytic cleavage at the furin site. Once cleaved, the resulting form is said to be the “latent” form of TGFβ1. The “latent” complex remains non-covalently associated until further activation trigger, such as integrin-driven activation event. The proTGFβ1 complex is comprised of dimeric TGFβ1 pro-protein polypeptides, linked with disulfide bonds. The latent dimer complex is covalently linked to a single presenting molecule via the cysteine residue at position 4 (Cys4) of each of the proTGFβ1 polypeptides. The adjective “latent” may be used generally/broadly to describe the “inactive” state of TGFβ1, prior to integrin-mediated or other activation events. The proTGFβ1 polypeptide contains a prodomain (LAP) and a growth factor domain (SEQ ID NO: 119).

Regression (tumor regression): Regression of tumor or tumor growth can be used as an in vivo efficacy measure. For example, in preclinical settings, median tumor volume (MTV) and Criteria for Regression Responses Treatment efficacy may be determined from the tumor volumes of animals remaining in the study on the last day. Treatment efficacy may also be determined from the incidence and magnitude of regression responses observed during the study. Treatment may cause partial regression (PR) or complete regression (CR) of the tumor in an animal. Complete regression achieved in response to therapy (e.g., administration of a drug) may be referred to as “complete response” and the subject that achieves complete response may be referred to as a “complete responder”. Thus, complete response excludes spontaneous complete regression. In some embodiments of preclinical tumor models, a PR response is defined as the tumor volume that is 50% or less of its Day 1 volume for three consecutive measurements during the course of the study, and equal to or greater than 13.5 mm³ for one or more of these three measurements. In some embodiments, a CR response is defined as the tumor volume that is less than 13.5 mm³ for three consecutive measurements during the course of the study. In preclinical model, an animal with a CR response at the termination of a study may be additionally classified as a tumor-free survivor (TFS). The term “effective tumor control” may be used to refer to a degree of tumor regression achieved in response to treatment, where, for example, the tumor volume is reduced to <25% of the endpoint tumor volume in response to treatment. For instance, in a particular model, if the endpoint tumor volume is 2,000 mm³, effective tumor control is achieved if the tumor is reduced to less than 500 mm³. Therefore, effective tumor control encompasses complete regression, as well as partial regression that reaches the threshold reduction.

Resistance (to therapy): Resistance to a particular therapy (such as CBT) may be due to the innate characteristics of the disease such as cancer (“primary resistance”, i.e., present before treatment initiation), or due to acquired phenotypes that develop over time following the treatment (“acquired resistance”). Patients who do not show therapeutic response to a therapy (e.g., those who are non-responders or poorly responsive to the therapy) are said to have primary resistance or acquired resistance to the therapy. Patients who have never previously received a treatment and do not show a therapeutic response to the treatment are said to have primary resistance. Patients who initially show therapeutic response to a therapy but later lose effects (e.g., progression or recurrence despite continued therapy) are said to have acquired resistance to the therapy. In the context of immunotherapy, such resistance can indicate immune escape.

Response Evaluation Criteria in Solid Tumors (RECIST) and iRECIST: RECIST is a set of published rules that define when tumors in cancer patients improve (“respond”), stay the same (“stabilize”), or worsen (“progress”) during treatment. The criteria were published in February 2000 by an international collaboration including the European Organisation for Research and Treatment of Cancer (EORTC), National Cancer Institute of the United States, and the National Cancer Institute of Canada Clinical Trials Group. Subsequently, a revised version of the RECIST guideline (RECIST v 1.1) has been widely adapted (see: Eisenhauera et al., (2009), “New response evaluation criteria in solid tumours: Revised RECIST guideline (version 1.1)” Eur J Cancer 45: 228-247, incorporated herein).

Response criteria are as follows: Complete response (CR): Disappearance of all target lesions; Partial response (PR): At least a 30% decrease in the sum of the LD of target lesions, taking as reference the baseline sum LD; Stable disease (SD): Neither sufficient shrinkage to qualify for PR nor sufficient increase to qualify for PD, taking as reference the smallest sum LD since the treatment started; Progressive disease (PD): At least a 20% increase in the sum of the LD of target lesions, taking as reference the smallest sum LD recorded since the treatment started or the appearance of one or more new lesions.

On the other hand, iRECIST provides a modified set of criteria that takes into account immune-related response (see: www.ncbi.nlm.nih.gov/pmc/articles/PMC5648544/contents of which are incorporated herein by reference). The RECIST and iRECIST criteria are standardized, may be revised from time to time as more data become available, and are well understood in the art.

Response rate: The term response rate (as in “low response rates”) as used herein carries the ordinary meaning as understood by the skilled person in medicine, such as oncologists. A response rate is the proportion (e.g., fraction or percentage) of subjects in a patient population who shows clinical improvement upon receiving a treatment (e.g., pharmacological intervention) and may include complete response and partial response. In oncology, clinical improvement may include tumor shrinkage (e.g., partial response) or disappearance (e.g., complete response). When used as a clinical endpoint for clinical trials of cancer treatments, this is typically expressed as the objective response rate (ORR). The FDA defines ORR as the proportion of patietns with tumor size reduction of a predefined amount and for a minimum time period. See: “Clinical Trial Endpoints for the Approval of Cander Drugs and Biologics—Guidance for Industry” published by U.S. Department of Health and Human Services, Food and Drug Administration, Oncology Center of Excellence, Center for Drug Evaluation and Research (CDER), Center for Biologics Evaluation and Research (CBER), the contents of which is incorporated herein by reference.

Solid tumor The term “solid tumor” refers to proliferative disorders resulting in an abnormal growth or mass of tissue that usually does not contain cysts or liquid areas. Solid tumors may be benign (non-cancerous), or malignant (cancerous). Solid tumors include tumors of advanced malignancies, such as locally advanced solid tumors and metastatic cancer. Solid tumors are typically comprised of multiple cell types, including, without limitation, cancerous (malignant) cells, stromal cells such as CAFs, and infiltrating leukocytes, such as macrophages, MDSCs and lymphocytes. Solid tumors to be treated with an isoform-selective inhibitor of TGFβ1, such as those described herein, are typically TGFβ31-positive (TGFβ1+) tumors, which may include multiple cell types that produce TGFβ1. In certain embodiments, the TGFβ1+ tumor may also co-express TGFβ3 (i.e., TGFβ3-positive). For example, certain tumors are TGFβ1/3-co-dominant. In some embodiments, such tumors are caused by cancer of epithelial cells, e.g., carcinoma. In certain embodiments, such tumors include ovarian cancer, breast cancer, bladder cancer, pancreatic cancer, e.g., pancreatic adenocarcinoma, prostate cancer, e.g., prostate adenocarcinoma, melanoma, e.g., skin cutaneous melanoma, lung cancer, e.g., lung squamous cell carcinoma and lung adenocarcinoma, liver cancer (e.g., liver hepatocellular carcinoma), uterine cancer, e.g., uterine corpus endometrial carcinoma, kidney cancer, e.g., renal clear cell carcinoma, head and neck cancer, e.g., head and neck squamous cell carcinoma, colon cancer, e.g., colon adenocarcinoma, esophageal carcinoma, and tenosynovial giant cell tumor (TGCT). In some embodiments, a solid tumor treated herein, such as one or more of those listed above, exhibits elevated TGFβ1 expression as compared to other tumor types and exhibits a reduced responsiveness to mainline therapy, e.g., genotoxic therapy. Accordingly, TGFβ inhibitors (e.g., Ab6) may be used in conjunction with one or more genotoxic therapies (e.g., chemotherapy and/or radiation therapy, including radiotherapeutic agents) to treat such cancer in a subject.

Specific binding: As used herein, the term “specific binding” or “specifically binds” means that an antibody, or antigen binding portion thereof, exhibits a particular affinity for a particular structure (e.g., an antigenic determinant or epitope) in an antigen (e.g., a K_{D} measured by Biacore®). In some embodiments, an antibody, or antigen binding portion thereof, specifically binds to a target, e.g., TGFβ1, if the antibody has a K_{D} for the target of at least about 10⁻⁸ M, 10⁻⁹ M, 10⁻¹⁰ M, 10⁻¹¹ M, 10⁻¹² M, or less. In some embodiments, the term “specific binding to an epitope of proTGFβ1”, “specifically binds to an epitope of proTGFβ1”, “specific binding to proTGFβ1”, or “specifically binds to proTGFβ1” as used herein, refers to an antibody, or antigen binding portion thereof, that binds to proTGFβ1 and has a dissociation constant (K_{D}) of 1.0×10⁻⁸ M or less, as determined by suitable in vitro binding assays, such as surface plasmon resonance and Biolayer Interferometry (BLI). In preferred embodiments, kinetic rate constants (e.g., K_{D}) are determined by surface plasmon resonance (e.g., a Biacore system). In one embodiment, an antibody, or antigen binding portion thereof, can specifically bind to both human and a non-human (e.g., mouse) orthologues of proTGFβ1. In some embodiments, an antibody may also “selectively” (i.e., “preferentially”) bind a target antigen if it binds that target with a comparatively greater strength than the strength of binding shown to other antigens, e.g., a 10-fold, 100-fold, 1000-fold, or greater comparative affinity for a target antigen (e.g., TGFβ1) than for a non-target antigen (e.g., TGFβ2 and/or TGFβ3). In preferred embodiments, an isoform-selective inhibitor exhibits no detectable binding or potency towards other isoforms or counterparts. In some embodiments, an antibody that binds specifically to a set of antigens may have high affinity toward said antigens but may not distinguish said antigens from one another (i.e., the antibody is specific but not selective). In some embodiments, an antibody that binds to an antigen with a particularly high affinity as compared to other antigens may be considered selective for said antigen. For instance, an antibody that binds to antigen X with 1000-fold higher affinity as compared to antigen Y may be considered an antibody that is selective for antigen X over antigen Y. In the context of the present disclosure, “an antibody that specifically binds an antigen with high affinity” generally refers to a K_{D} of 1.0×10⁻⁸ M or less.

Subject: The term “subject” in the context of therapeutic applications refers to an individual who receives or is in need of clinical care or intervention, such as treatment, diagnosis, etc. Suitable subjects include vertebrates, including but not limited to mammals (e.g., human and non-human mammals). Where the subject is a human subject, the term “patient” may be used interchangeably. In a clinical context, the term “a patient population” or “patient subpopulation” is used to refer to a group of individuals that falls within a set of criteria, such as clinical criteria (e.g., disease presentations, disease stages, susceptibility to certain conditions, responsiveness to therapy, etc.), medical history, health status, gender, age group, genetic criteria (e.g., carrier of certain mutation, polymorphism, gene duplications, DNA sequence repeats, etc.) and lifestyle factors (e.g., smoking, alcohol consumption, exercise, etc.).

Target engagement: As used herein, the term target engagement refers to the ability of a molecule (e.g., TGFβ inhibitor) to bind to its intended target in vivo (e.g., endogenous TGFβ). In case of activation inhibitors, the intended target can be a large latent complex.

TGFβ1-related indication: A “TGFβ1-related indication” is a TGFβ1-associated disorder and means any disease or disorder, and/or condition, in which at least part of the pathogenesis and/or progression is attributable to TGFβ1 signaling or dysregulation thereof. Certain TGFβ1-associated disorders are driven predominantly by the TGFβ1 isoform. Subjects having a TGFβ1-related indication may benefit from inhibition of the activity and/or levels TGFβ1. Certain TGFβ1-related indications are driven predominantly by the TGFβ1 isoform. TGFβ1-related indications include, but are not limited to: fibrotic conditions (such as organ fibrosis, and fibrosis of tissues involving chronic inflammation), proliferative disorders (such as cancer, e.g., solid tumors and myelofibrosis), disease associated with ECM dysregulation (such as conditions involving matrix stiffening and remodeling), disease involving mesenchymal transition (e.g., EndMT and/or EMT), disease involving proteases, disease with aberrant gene expression of certain markers described herein. These disease categories are not intended to be mutually exclusive.

TGFβ inhibitor: The term “TGFβ inhibitor” refers to any agent capable of antagonizing biological activities, signaling or function of TGFβ growth factor (e.g., TGFβ1, TGFβ2 and/or TGFβ3). The term is not intended to limit its mechanism of action and includes, for example, neutralizing inhibitors, receptor antagonists, soluble ligand traps, TGFβ activation inhibitors, and integrin inhibitors (e.g., antibodies that bind to αVβ1, αVβ3, αVβ5, αVβ6, αVβ8, α5β1, αIIbβ3, or α8β1 integrins, and inhibit downstream activation of TGFβ. e.g., selective inhibition of TGFβ1 and/or TGFβ3). The term encompasses TGFβ inhibitors that are isoform-selective and non-selective inhibitors. The latter include, for example, small molecule receptor kinase inhibitors (e.g., ALK5 inhibitors), antibodies (such as neutralizing antibodies) that preferentially bind two or more isoforms, and engineered constructs (e.g., fusion proteins) comprising a ligand-binding moiety. In certain embodiments, these antibodies may include or may be engineered to include a mutation or modification that causes an extended half-life of the antibody. In some embodiments, such mutations or modifications may be within the Fc domain of the antibodies (e.g., Fc-modified antibodies). In some embodiments, the mutation is so-called YTE mutation. TGFβ inhibitors also include antibodies that are capable of reducing the availability of latent proTGFβ which can be activated in the niche, for example, by inducing antibody-dependent cell mediated cytotoxicity (ADCC), and/or antibody-dependent cellular phagocytosis (ADPC), as well as antibodies that result in internalization of cell-surface complex comprising latent proTGFβ, thereby removing the precursor from the plasma membrane without depleting the cells themselves. Internalization may be a suitable mechanism of action for LRRC33-containing protein complexes (such as human LRRC33-proTGFβ1) which results in reduced levels of cells expressing LRRC33-containing protein complexes on cell surface.

The “TGFβ family” is a class within the TGFβ superfamily and in human contains three members: TGFβ1, TGFβ2, and TGFβ3, which are structurally similar. The three growth factors are known to signal via the same receptors.

TGFβ1-positive cancer/tumor The term, as used herein, refers to a cancer/tumor with aberrant TGFβ1 expression (overexpression). Many human cancer/tumor types show predominant expression of the TGFβ1 (note that “TGFβ” is sometimes used to refer to the gene as opposed to protein) isoform. In some cases, such cancer/tumor may show co-dominant expression of another isoform, such as TGFβ3. A number of epithelial cancers (e.g., carcinoma) may co-express TGFβ1 and TGFβ3. Within the tumor environment of TGFβ1-positive tumors, TGFβ1 may arise from multiple sources, including, for example, cancer cells, tumor-associated macrophages (TAMs), cancer-associated fibroblasts (CAFs), regulatory T cells (Tregs), myeloid-derived suppressor cells (MDSCs), and the surrounding extracellular matrix (ECM). In the context of the present disclosure, preclinical cancer/tumor models that recapitulate human conditions are TGFβ1-positive cancer/tumor.

Therapeutic window: The term “therapeutic window” refers to a dosage range that produces therapeutic response without causing significant/observable/unacceptable adverse effect (e.g., within adverse effects that are acceptable or tolerable) in subjects. Therapeutic window may be calculated as a ratio between minimum effective concentrations (MEC) to the minimum toxic concentrations (MTC). To illustrate, a TGFβ1 inhibitor that achieves in vivo efficacy at 10 mg/kg dosage and shows tolerability or acceptable toxicities at 100 mg/kg provides at least a 10-fold (e.g., 10×) therapeutic window. By contrast, a pan-inhibitor of TGFβ that is efficacious at 10 mg/kg but causes adverse effects at less than the effective dose is said to have “dose-limiting toxicities.” Generally, the maximally tolerated dose (MTD) may set the upper limit of the therapeutic window. For example, Ab6 was shown to be efficacious at dosage ranging between about 3-30 mg/kg/week and was also shown to be free of observable toxicities associated with pan-inhibition of TGFβ at dosage of at least 100 or 300 mg/kg/week for 4 weeks in rats or non-human primates. Based on this, Ab6 shows at minimum a 3.3-fold and up to 100-fold therapeutic window. In some embodiments, the concept of therapeutic window may be expressed in terms of safety factors.

Toxicity: As used herein, the term “toxicity” or “toxicities” refers to unwanted in vivo effects in subjects (e.g., patients) associated with a therapy administered to the subjects (e.g., patients), such as undesirable side effects and adverse events. “Tolerability” refers to a level of toxicities associated with a therapy or therapeutic regimen, which can be reasonably tolerated by patients, without discontinuing the therapy due to the toxicities. Typically, toxicity/toxicology studies are carried out in one or more preclinical models prior to clinical development to assess safety profiles of a drug candidate (e.g., monoclonal antibody therapy). Toxicity/toxicology studies may help determine the “no-observed-adverse-effect level (NOAEL)” and the “maximally tolerated dose (MTD)” of a test article, based on which a therapeutic window may be deduced. Preferably, a species that is shown to be sensitive to the particular intervention should be chosen as a preclinical animal model in which safety/toxicity study is to be carried out. In case of TGFβ inhibition, suitable species include rats, dogs, and cynos. Mice are reported to be less sensitive to pharmacological inhibition of TGFβ and may not reveal toxicities that are potentially dangerous in other species, including human, although certain studies report toxicities observed with pan-inhibition of TGFβ in mice. To illustrate in the context of the present disclosure, the NOAEL for Ab6 in rats was the highest dose evaluated (100 mg/kg), suggesting that the MTD is >100 mg/kg, based on a four-week toxicology study. The MTD of Ab6 in non-human primates is >300 mg/kg based on a four-week toxicology study.

For determining NOAELs and MTDs, preferably, a species that is shown to be sensitive to the particular intervention should be chosen as a preclinical animal model in which safety/toxicology study is to be carried out. In case of TGFβ inhibition, suitable species include, but are not limited to, rats, dogs, and cynos. Mice are reported to be less sensitive to pharmacological inhibition of TGFβ and may not reveal toxicities that are potentially serious or dangerous in other species, including human.

Treat/treatment: The term “treat” or “treatment” includes therapeutic treatments, prophylactic treatments, and applications in which one reduces the risk that a subject will develop a disorder or other risk factor. Thus the term is intended to broadly mean: causing therapeutic benefits in a patient by, for example, enhancing or boosting the body's immunity; reducing or reversing immune suppression; reducing, removing or eradicating harmful cells or substances from the body; reducing disease burden (e.g., tumor burden); preventing recurrence or relapse; prolonging a refractory period, and/or otherwise improving survival. The term includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In the context of combination therapy, the term may also refer to: i) the ability of a second therapeutic to reduce the effective dosage of a first therapeutic so as to reduce side effects and increase tolerability; ii) the ability of a second therapy to render the patient more responsive to a first therapy; and/or iii) the ability to effectuate additive or synergistic clinical benefits.

Tumor-associated macrophage (TAM): TAMs are polarized/activated macrophages with pro-tumor phenotypes (M2-like macrophages). TAMs can be either marrow-originated monocytes/macrophages recruited to the tumor site or tissue-resident macrophages which are derived from erythro-myeloid progenitors. Differentiation of monocytes/macrophages into TAMs is influenced by a number of factors, including local chemical signals such as cytokines, chemokines, growth factors and other molecules that act as ligands, as well as cell-cell interactions between the monocytes/macrophages that are present in the niche (tumor microenvironment). Generally, monocytes/macrophages can be polarized into so-called “M1” or “M2” subtypes, the latter being associated with more pro-tumor phenotype. In a solid tumor, up to 50% of the tumor mass may correspond to macrophages, which are preferentially M2-polarized. Among tumor-associated monocytes and myeloid cell populations, M1 macrophages typically express cell surface HLA-DR, CD68 and CD86, while M2 macrophages typically express cell surface HLA-DR, CD68, CD163 and CD206. Tumor-associated, M2-like macrophages (such as M2c and M2d subtypes) can express cell surface LRRC33 and/or LRRC33-proTGFβ1.

Tumor microenvironment: The term “tumor microenvironment (TME)” refers to a local disease niche, in which a tumor (e.g., solid tumor) resides in vivo. The TME may comprise disease-associated molecular signature (a set of chemokines, cytokines, etc.), disease-associated cell populations (such as TAMs, CAFs, MDSCs, etc.) as well as disease-associated ECM environments (alterations in ECM components and/or structure).

Variable region: The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

Other than in the operating examples, or where otherwise indicated, all numbers expressing quantities of ingredients or reaction conditions used herein should be understood as modified in all instances by the term “about.” The term “about” means±10% of the recited value.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, e.g., 10-20, 1-10, 30-40, etc.

### Transforming Growth Factor-Beta (TGFβ)

The Transforming Growth Factor-beta (TGFβ) activities and subsequent partial purification of the soluble growth factors were first described in the late 1970's to early 1980's, with which the TGFβ field began some 40 years ago. To date, 33 gene products have been identified that make up the large TGFβ superfamily. The TGFβ superfamily can be categorized into at least three subclasses by structural similarities: TGFβs, Growth-Differentiation Factors (GDFs) and Bone-Morphogenetic Proteins (BMPs). The TGFβ subclass is comprised of three highly conserved isoforms, namely, TGFβ1, TGFβ2 and TGFβ3, which are encoded by three separate genes in human.

The TGFβs are thought to play key roles in diverse processes, such as inhibition of cell proliferation, extracellular matrix (ECM) remodeling, and immune homeostasis. The importance of TGFβ1 for T cell homeostasis is demonstrated by the observation that TGFβ1−/− mice survive only 3-4 weeks, succumbing to multi-organ failure due to massive immune activation (Kulkarni, A. B., et al., Proc Natl Acad Sci USA, 1993. 90(2): p. 770-4; Shull, M. M., et al., Nature, 1992. 359(6397): p. 693-9). The roles of TGFβ2 and TGFβ3 are less clear. Whilst the three TGFβ isoforms have distinct temporal and spatial expression patterns, they signal through the same receptors, TGFβRI and TGFβRII, although in some cases, for example for TGFβ2 signaling, type III receptors such as betaglycan are also required (Feng, X. H. and R. Derynck, Annu Rev Cell Dev Biol, 2005. 21: p. 659-93; Massague, J., Annu Rev Biochem, 1998. 67: p. 753-91). Ligand-induced oligomerization of TGFβRI/II triggers the phosphorylation of SMAD transcription factors, resulting in the transcription of target genes, such as Col1a1, Col3a1, ACTA2, and SERPINE1 (Massague, J., J. Seoane, and D. Wotton, Genes Dev, 2005. 19(23): p. 2783-810). SMAD-independent TGFβ signaling pathways have also been described, for example in cancer or in the aortic lesions of Marfan mice (Derynck, R. and Y. E. Zhang, Nature, 2003. 425(6958): p. 577-84; Holm, T. M., et al., Science, 2011. 332(6027): p. 358-61).

The biological importance of the TGFβ pathway in humans has been validated by genetic diseases. Camurati-Engelman disease results in bone dysplasia due to an autosomal dominant mutation in the TGFB1 gene, leading to constitutive activation of TGFβ1 signaling (Janssens, K., et al., J Med Genet, 2006. 43(1): p. 1-11). Patients with Loeys/Dietz syndrome carry autosomal dominant mutations in components of the TGFβ signaling pathway, which cause aortic aneurism, hypertelorism, and bifid uvula (Van Laer, L., H. Dietz, and B. Loeys, Adv Exp Med Biol, 2014. 802: p. 95-105). As TGFβ pathway dysregulation has been implicated in multiple diseases, several drugs that target the TGFβ pathway have been developed and tested in patients, but with limited success.

Dysregulation of the TGFβ signaling has been associated with a wide range of human diseases. Indeed, in a number of disease conditions, such dysregulation may involve multiple facets of TGFβ function. Diseased tissue, such as fibrotic and/or inflamed tissues and tumors, may create a local environment in which TGFβ activation can cause exacerbation or progression of the disease, which may be at least in part mediated by interactions between multiple TGFβ-responsive cells, which are activated in an autocrine and/or paracrine fashion, together with a number of other cytokines, chemokines and growth factors that play a role in a particular disease setting.

For example, a tumor microenvironment (TME) contains multiple cell types expressing TGFβ1, such as activated myofibroblast-like fibroblasts, stromal cells, infiltrating macrophages, MDSCs and other immune cells, in addition to cancer (i.e., malignant) cells. Thus, the TME represents a heterogeneous population of cells expressing and/or responsive to TGFβ1 but in association with more than one types of presenting molecules, e.g., LTBP1, LTBP3, LRRC33 and GARP, within the niche.

Advances in immunotherapy have transformed the effective treatment landscape for a growing number of cancer patients. Most prominent are the checkpoint blockade therapies (CBT), which have now become part of standard of care regimens for an increasing number of cancers. While profound and durable responses to CBT have been observed across a growing number of cancer types, it is now clear that a significant fraction of tumors appear to be refractory to CBT even at the outset of treatment, hence pointing to primary resistance as a major challenge to enabling many patients' immune systems to target and eliminate tumor cells. Efforts to understand and address the underlying mechanisms conferring primary resistance to CBT have been undertaken in order to broaden treatment efficacy for a greater number of patients. However, this enthusiasm has been curbed by lackluster clinical trial results and failures when combining CBTs with agents known to affect the same tumor type or to modulate seemingly relevant components of the immune system. A likely reason is that a clear mechanistic rationale for the given combination is often not rooted in clinically-derived data, and has thus led to uncertain and confounding outcomes in trials intended to enhance approved single-agent therapies. It has become clear that the design of combination immunotherapy should be rooted in scientific evidence of relevance to underlying tumor and immune system biology.

Recently, a phenomenon referred to as “immune exclusion” was coined to describe a tumor environment from which anti-tumor effector T cells (e.g., CD8+ T cells) are kept away (hence “excluded”) by immunosuppressive local cues. More recently, a number of retrospective analyses of clinically-derived tumors have implicated TGFβ pathway activation in mediating primary resistance to CBT. For example, transcriptional profiling and analysis of pretreatment melanoma biopsies revealed an enrichment of TGFβ-associated pathways and biological processes in tumors that are non-responsive to anti-PD-1 CBT. In an immune-excluded tumor, effector cells, which would otherwise be capable of attacking cancer cells by recognizing cell-surface tumor antigens, are prevented from gaining access to the site of cancer cells. In this way, cancer cells evade host immunity and immuno-oncologic therapeutics, such as checkpoint inhibitors, that exploit and rely on such immunity. Indeed, such tumors show resistance to checkpoint inhibition, such as anti-PD-1 and anti-PD-L1 antibodies, presumably because target T cells are blocked from entering the tumor hence failing to exert anti-cancer effects.

A number of retrospective analyses of clinically-derived tumors points to TGFβ pathway activation in mediating primary resistance to CBT. For example, transcriptional profiling and analysis of pretreatment melanoma biopsies revealed an enrichment of TGFβ-associated pathways and biological processes in tumors that are non-responsive to anti-PD-1 CBT. More recently, similar analyses of tumors from metastatic urothelial cancer patients revealed that lack of response to PD-L1 blockade with atezolizumab was associated with transcriptional signatures of TGFβ signaling, particularly in tumors wherein CD8+ T cells appear to be excluded from entry into the tumor. The critical role of TGFβ signaling in mediating immune exclusion resulting in anti-PD-(L)1 resistance has been verified in the EMT-6 syngeneic mouse model of breast cancer. While the EMT-6 tumors are weakly responsive to treatment with an anti-PD-L1 antibody, combining this checkpoint inhibitor with 1D11, an antibody that blocks the activity of all TGFβ isoforms, resulted in a profound increase in the frequency of complete responses when compared to treatment with individual inhibitors. The synergistic antitumor activity is proposed to be due to a change in cancer-associated fibroblast (CAF) phenotype and a breakdown of the immune excluded phenotype, resulting in infiltration of activated CD8+ T cells into the tumors. Similar results were found in a murine model of colorectal cancer and metastasis using a combination of an anti-PD-L1 antibody with galunisertib, a small molecule inhibitor of the type I TGFβ receptor ALK5 kinase. Collectively, these findings suggest that inhibiting the TGFβ pathway in CBT-resistant tumors could be a promising approach to improve or increase the number of clinical responses to CBT. While recent work has implicated a relationship between TGFβ pathway activation and primary CBT resistance, TGFβ signaling has long been linked to features of cancer pathogenesis. As a potent immunosuppressive factor, TGFβ prevents antitumor T cell activity and promotes immunosuppressive macrophages. Malignant cells often become resistant to TGFβ signaling as a mechanism to evade its growth and tumor-suppressive effects. TGFβ activates CAFs, inducing extracellular matrix production and promotion of tumor progression. Finally, TGFβ induces EMT, thus supporting tissue invasion and tumor metastases.

Mammals have distinct genes that encode and express the three TGFβ growth factors, TGFβ1, TGFβ2, and TGFβ3, all of which signal through the same heteromeric TGFβ receptor complex. Despite the common signaling pathway, each TGFβ isoform appears to have distinct biological functions, as evidenced by the non-overlapping TGFβ knockout mouse phenotypes. All three TGFβ isoforms are expressed as inactive prodomain-growth factor complexes, in which the TGFβ prodomain, also called latency-associated peptide (LAP), wraps around its growth factor and holds it in a latent, non-signaling state. Furthermore, latent TGFβ is co-expressed with latent TGFβ-binding proteins and forms large latent complexes (LLCs) through disulfide linkage. Association of latent TGFβ with Latent TGFβ Binding Protein-1 (LTBP1) or LTBP3 enables tethering to extracellular matrix, whereas association to the transmembrane proteins GARP or LRRC33 enables elaboration on the surface of Tregs or macrophages, respectively. In vivo, latent TGFβ1 and latent TGFβ3 are activated by a subset of aV integrins, which bind a consensus RGD sequence on LAP, triggering a conformational change to release the growth factor. The mechanism by which latent TGFβ2 is activated is less clear as it lacks a consensus RGD motif. TGFβ1 release by proteolytic cleavage of LAP has also been implicated as an activation mechanism, but its biological relevance is less clear.

Although the pathogenic role of TGFβ activation is clear in several disease states, it is equally clear that therapeutic targeting of the TGFβ pathway has been challenging due to the pleiotropic effects that result from broad and sustained pathway inhibition. For example, a number of studies have shown that small molecule-mediated inhibition of the TGFβ type I receptor kinase ALK5 (TGFBR1) or blockade of all three highly related TGFβ growth factors with a high-affinity antibody resulted in severe cardiac valvulopathies in mice, rats and dogs. These “pan”-TGFβ approaches that block all TGFβ signaling therefore have a very narrow therapeutic window, which has proven to be an impediment to the treatment of a number of disease-relevant processes with very high unmet medical need. No TGFβ-targeting therapy has been approved to date and clinical trial results with such modalities have largely been disappointing, likely due to the use of what proved to be inefficacious dosing regimens that were required in order to accommodate safety concerns.

All references cited herein are incorporated by reference for any purpose. Where a reference and the specification conflict, the specification will control. It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

### Methods of Treatment and Biomarkers of Therapeutic Efficacy

The subject matter of the present disclosure generally relates to the disclosure of PCT/2021/012969 filed Jan. 11, 2021, the entire content of which is incorporated by reference herein.

### Circulating/Circulatory MDSCs as a Biomarker

MDSCs are a heterogeneous population of cells named for their myeloid origin and their main immune suppressive function (Gabrilovich. Cancer Immunol Res. 2017 January; 5(1): 3-8). MDSCs generally exhibit high plasticity and strong capacity to reduce cytotoxic functions of T cells and natural killer (NK) cells, including their ability to promote T regulatory cell (Treg) expansion and in turn suppress T effector cell function (Gabrilovich et al., Nat Rev Immunol. (2012) 12:253-68). MDSCs are typically classified into two subsets, monocytic (m-MDSCs) and granulocytic (G-MDSCs or PMN-MDSCs), based on their expression of surface markers (Consonni et al., Front Immunol. 2019 May 3; 10:949). Suppressive G-MDSCs can be characterized by their production of reactive oxygen species (ROS) as the major mechanism of immune suppression. In contrast, M-MDSCs mediate immune suppression primarily by upregulating the inducible nitric oxide synthase gene (iNOS) and produce nitric oxide (NO) as well as an array of immune suppressive cytokines (Youn and Garilovich, Eur J Immunol. 2010 November; 40(11): 2969-2975).

MDSCs have been implicated in various diseases, such as chronic inflammation, infection, autoimmune diseases, and graft-versus-host diseases. In recent years, MDSCs have become an immune population of interest in cancer due to their role in inducing T cell tolerance through checkpoint blockade molecules such as the programmed death-ligand 1 (PD-L1) and the cytotoxic T-lymphocyte antigen 4 (CTLA4) (Trovato et al., J Immunother Cancer. 2019 Sep. 18; 7(1):255). Furthermore, MDSCs have generally been characterized as favoring tumor progression by mechanisms in addition to immune suppression, including promoting tumor angiogenesis. Studies to date have focused on MDSCs present in tumor biopsies, given their propensity to enrich around inflamed tissue. (Passro et al., Clin Transl Oncol. 2019 Jun. 28.; Ai et al., BMC Cancer. 2018 Dec. 5; 18(1):1220; Nakamura. Front Med (Lausanne). 2019; 6: 119). However, such studies had not been reported in the literature to have elucidated a clear relationship between MDSC levels and therapeutic response. For instance, low baseline monocytic MDSC frequency was shown to correlate poorly with treatment benefits (Pico de Coaña et al., Oncotarget. 2017 Mar. 28; 8(13): 21539-21553).

Many human cancers (e.g., solid tumors) are known to show elevated levels of MDSCs in biopsies from patients, as compared to healthy controls (reviewed, for example, in Elliott et al., (2017) Frontiers in Immunology, Vol. 8, Article 86). These human cancers include but are not limited to bladder cancer, colorectal cancer, prostate cancer, breast cancer, glioblastoma, hepatocellular carcinoma, head and neck squamous cell carcinoma, lung cancer, melanoma, NSCLC, ovarian cancer, pancreatic cancer, and renal cell carcinoma. The compositions and methods according to the present disclosure may be applied to one or more of these cancers.

Previously, it was demonstrated by Applicant that immunosuppressive tumors contain elevated levels of tumor-infiltrating or intratumoral MDSCs, also referred to as tumor-associated MDSCs, and evidence indicated that this was inversely correlated with anti-tumor immunity in a TGFβ31-dependent manner. Data provided in PCT/US2019/041373, the content of which is hereby incorporated in its entirety. These data suggested that probing tumor-associated immune cells, by, for example, biopsies, can be useful for characterizing anti-tumor effects in cancer patients. Further, Applicant made a surprising finding that relatively simple and noninvasive blood tests may provide equivalent information, leading to the recognition that pharmacological effects of TGFβ1 inhibition on overcoming an immunosuppressive phenotype can be determined by measuring circulating MDSC levels.

The present disclosure includes the finding that circulating MDSC levels (including gMDSCs and/or mMDSCs) may be determined by detecting or measuring LRRC33-positive cells in a blood sample, identifying LRRC33 as a novel blood-based biomarker for circulating MDSCs. For example, LRRC33-positive cells in a blood sample collected from a patient (such as cancer patient) may be detected or measured by a FACS-based assay using an antibody that binds cell-surface LRRC33. In some embodiments, the LRRC33-expressing cells in a blood sample collected from a subject having cancer are G-MDSCs. While MDSCs are derived from bone marrow-originated monocytes, cell-surface expression of LRRC33 appears to be narrowly restricted to MDSCs, and not monocytes, in circulation. This recognition raises a new possibility of using LRRC33 as a blood-based marker for circulating MDSCs. In some embodiments, LRRC33 expression may be determined by any of the antibodies disclosed in WO/2018/208888 and WO/2018/081287, the contents of which are incorporated herein in their entirety. Applicant has now established a correlation between circulatory MDSC levels and tumor-associated MDSC levels. Together with the finding that circulatory MDSCs appear to show robust and uniform cell-surface LRRC33 expression, determination of LRRC33 levels measured in blood samples may serve as an effective surrogate to assess tumor immune-phenotype, such as immunosuppression, without the need for more invasive procedures such as tumor biopsy.

In various embodiments, the present disclosure provides methods of treating cancer, predicting, or determining efficacy, and/or confirming pharmacological response by monitoring the levels of circulating MDSCs in a sample obtained from a patient (e.g., in the blood or a blood component of a patient) receiving a TGFβ inhibitor, e.g., a TGFβ1-selective inhibitor (such as a selective pro- or latent-TGFβ1 inhibitor, e.g., Ab6), isoform-non-selective TGFβ inhibitors (such as low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors), and/or an integrin inhibitor (and integrin inhibitors (e.g., antibodies that bind to αVβ1, αVβ3, αVβ5, αVβ6, αVβ8, α5β1, αIIbβ3, or α8β1 integrins, and inhibit downstream activation of TGFβ. e.g., selective inhibition of TGFβ1 and/or TGFβ3). Exemplary integrin inhibitors include the anti-αVβ8 integrin antibodies provided in WO2020051333, the disclosure of which is incorporated by reference. In various embodiments disclosed herein, the circulating MDSCs may be measured within 1, 2, 3, 4, 5, 6, or 7 days, or within 1, 2, 3, 4, 5, 6, 7, 8, 9, or 10 weeks (e.g., preferably less than 6 weeks) following administration of a treatment to a subject, e.g., administration of a therapeutic dose of a TGFβ inhibitor.

In certain embodiments, the TGFβ treatment may be administered alone or in conjunction with an additional cancer therapy. The treatment may be administered to subjects with an immunosuppressive cancer or a myeloproliferative disorder. In some embodiments, the TGFβ inhibitor is a TGFβ1-selective antibody or antigen-binding fragment thereof encompassed in the current disclosure (e.g., Ab6). In some embodiments, the TGFβ1-selective antibody or antigen-binding fragment does not inhibit TGFβ2 and TGFβ3 at a therapeutically effective dose. In some embodiments, the TGFβ inhibitor is an isoform-non-selective TGFβ inhibitor (such as low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors). In some embodiments, the TGFβ inhibitor is an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). Exemplary integrin inhibitors include the anti-αVβ8 integrin antibodies provided in WO2020051333, the disclosure of which is incorporated by reference. In some embodiments, the additional cancer therapy may include chemotherapy, radiation therapy (including radiotherapeutic agents), cancer vaccine or immunotherapy including checkpoint inhibitor therapies such as anti-PD-1, anti-PD-L1, and anti-CTLA-4 antibodies. In some embodiments, the checkpoint inhibitor therapy is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (e.g., ABBV-181); and durvalumab (e.g., Imfinzi®). In preferred embodiments, a combination cancer therapy comprises Ab6 and at least one checkpoint inhibitor (such as those listed above). Thus, in some embodiments, a combination of Ab6 and a checkpoint inhibitor is used for the treatment of cancer in a human patient in amounts effective to treat the cancer. In some embodiments, the TGFβ treatment may further or alternatively include a second checkpoint inhibitor. In some embodiments, the TGFβ treatment may further or alternatively include a chemotherapy (e.g., a genotoxic therapy or radiation therapy).

For example, without being bound by theory, evidence suggests that overactive TGFβ pathways may correlate with unresponsiveness of a tumor to genotoxic therapies, such as chemotherapy and radiation therapy (Liu et al., Sci Transl Med. 2021 Feb. 10; 13(580):eabc4465). This is observed across multiple cancer types, e.g., cancers of the epithelia, e.g., carcinoma. In certain embodiments, such cancer types include ovarian cancer, breast cancer, bladder cancer, pancreatic cancer, e.g., pancreatic adenocarcinoma, prostate cancer, e.g., prostate adenocarcinoma, melanoma, e.g., skin cutaneous melanoma, lung cancer, e.g., lung squamous cell carcinoma and lung adenocarcinoma, liver cancer (e.g., liver hepatocellular carcinoma), uterine cancer, e.g., uterine corpus endometrial carcinoma, kidney cancer, e.g., renal clear cell carcinoma, head and neck cancer, e.g., head and neck squamous cell carcinoma, colon cancer, e.g., colon adenocarcinoma, esophageal carcinoma, and tenosynovial giant cell tumor (TGCT). In some embodiments, the cancer is a cancer having elevated TGFβ1 levels associated with ROS (e.g., elevated ROS). Without being bound by theory, ROS may induce an increase in TGFβ levels (e.g., TGFβ1 levels) which may be reduced by a TGFβ inhibitor (e.g., a TGFβ1 inhibitor) disclosed herein.

Accordingly, TGFβ inhibitors (e.g., Ab6) may be used in conjunction with one or more genotoxic therapies (e.g., chemotherapy and/or radiation therapy, including radiotherapeutic agents) to treat such a cancer in a subject. In certain embodiments, such a cancer may have elevated TGFβ levels, e.g., elevated TGFβ activity, as indicated by direct measurement and/or one or more changes in downstream gene regulation (e.g., in one or more genes involved in DNA repair). For instance, a cancer, such as one of the cancers listed above, may have elevated TGFβ signaling as indicated by upregulation of one or more genes associated with non-homologous end joining (NHEJ), e.g., Cyclin Dependent Kinase Inhibitor 1A (CDKN1A), or downregulation of one or more genes relating to alternative end joining, e.g., LIG1 (DNA ligase 1), PARP1, and/or POLQ.

The present disclosure also provides methods of using measurements of circulating MDSCs in treating cancer in subjects administered a TGFβ inhibitor alone or in conjunction with an immunotherapy. Furthermore, the descriptions presented herein provide support for the circulating MDSC population as an early predictive marker of efficacy, particularly in cancer subjects treated with a TGFβ inhibitor and checkpoint inhibitor combination therapy, e.g., at a time point before other markers of treatment efficacy, such as a reduction in tumor volume, can be detected.

In certain embodiments, a TGFβ inhibitor, e.g., a TGFβ1-selective inhibitor such as Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) is administered concurrently (e.g., simultaneously), separately, or sequentially to a checkpoint inhibitor therapy such that the amount (e.g., dose) of TGFβ1 inhibition administered is sufficient to reduce circulating MDSC levels by at least 10%, at least 15%, at least 20%, at least 25%, or more, as compared to baseline MDSC levels. Circulating MDSC levels may be measured prior to or after each treatment or each dose of the TGFβ inhibitor such that a decrease of at least 10%, at least 15%, at least 20%, at least 25%, or more in circulating MDSC levels may be indicative or predictive of treatment efficacy. In some embodiments, the level of circulating MDSCs may be used to determine disease burden (e.g., as measured by a change in relative tumor volume before and after a treatment regimen). In some embodiments, the level of circulating mMDSCs may be used to determine disease burden (e.g., as measured by a change in relative tumor volume before and after a treatment regimen).

In certain embodiments, a decrease in circulating MDSC levels (e.g., mMDSC levels) may be indicative of a decrease in disease burden (e.g., a decrease in relative tumor volume). For instance, circulating MDSC levels (e.g., mMDSC levels) may be measured prior to and after the administration of a dose of TGF inhibitor (such as isoform-selective inhibitors, e.g., Ab6, isoform-non-selective TGFβ inhibitors, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ, e.g., selective inhibition of TGFβ1 and/or TGFβ3) and a reduction in the circulating MDSC levels may be indicative or predictive of pharmacological effects, e.g., of a reduction in disease burden (e.g., a reduction in relative tumor size).

In certain embodiments, circulating MDSC levels (e.g., circulating mMDSC levels) may be measured prior to and following administration of a first dose of a TGFβ inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). In some embodiments, administration of a first dose of TGFβ inhibitor e.g., Ab6, isoform-non-selective TGFβ inhibitors, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor, e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3 may be used to reduce tumor volume, such that administration of the TGFβ inhibitor reduces circulating MDSC levels by at least 10%, at least 20%, at least 25%, or more, as compared to circulating MDSC levels (e.g., circulating mMDSC levels) prior to administration.

In some embodiments, reduction in circulating MDSC levels (e.g., circulating mMDSC levels) is indicative or predictive of pharmacological effects and further warrants administration of a second or more dose(s) of the TGFβ inhibitor. In some embodiments, reduction in circulating mMDSC levels is indicative or predictive of pharmacological effects and further warrants administration of a second or more dose(s) of the TGFβ inhibitor. In some embodiments, the first dose of the TGFβ inhibitor is the very first dose of TGFβ inhibitor received by the patient. In some embodiments, the first dose of the TGFβ inhibitor is the first dose of a given treatment regimen comprising more than one dose of TGFβ inhibitor. In another embodiment, circulating MDSC levels (e.g., circulating mMDSC levels) may be measured prior to and after combination treatment comprising a TGFβ inhibitor (e.g., Ab6) and a checkpoint inhibitor therapy, administered concurrently (e.g., simultaneously), separately, or sequentially, and a reduction in the circulating MDSC levels is indicative or predictive of therapeutic efficacy. In some embodiments, the reduction of circulating MDSC levels following the combination treatment of a TGFβ inhibitor, such as a TGFβ1 inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3), and a checkpoint inhibitor therapy, may warrant continuation of treatment. In some embodiments, the reduction of circulating mMDSC levels following the combination treatment of a checkpoint inhibitor therapy and a TGFβ inhibitor, such as a TGFβ1 inhibitor, such as a TGFβ1-selective inhibitor, e.g., Ab6, or an isoform-non-selective inhibitor, e.g., a low molecular weight ALK5 antagonist, a neutralizing antibody that bind two or more of TGFβ1/2/3, e.g., GC1008 and variants, an antibody that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3), may warrant continuation of treatment. In some embodiments, the reduction of circulating gMDSC levels following the combination treatment of a checkpoint inhibitor therapy and a TGFβ inhibitor, such as a TGFβ1 inhibitor, such as a TGFβ31-selective inhibitor, e.g., Ab6, or an isoform-non-selective inhibitor, e.g., a low molecular weight ALK5 antagonist, a neutralizing antibody that bind two or more of TGFβ1/2/3, e.g., GC1008 and variants, an antibody that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3), may warrant continuation of treatment.

In certain embodiments of the present disclosure, levels of circulating MDSCs may be used to predict, determine, and monitor pharmacological effects of treatment comprising a dose of TGFβ inhibitor, such as a TGFβ31-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) administered alone or in conjunction with another cancer therapy such as a checkpoint inhibitor. In certain embodiments, a baseline circulating MDSC level may be measured before administering an initial treatment (e.g., a first dose of a TGFβ inhibitor). In certain embodiments, a low baseline circulating MDSC level is predictive of better response to the TGFβ inhibitor treatment. In certain embodiments, a low baseline circulating mMDSC level is predictive of better response to the TGFβ inhibitor treatment. In certain embodiments, a low baseline circulating mMDSC level is predictive of better response to the TGFβ inhibitor treatment. In certain embodiments, a patient administered the TGFβ inhibitor treatment has a low baseline circulating mMDSC level. In certain embodiments, circulating MDSCs may be measured within six weeks following administration of the initial treatment. In certain embodiments, circulating MDSC levels may be measured within thirty days following administration of the initial dose of TGFβ inhibitor. In some embodiments, MDSC levels may be measured within or at about three weeks following administration of the initial dose of TGFβ inhibitor. In some embodiments, MDSC levels may be measured within or at about two weeks following administration of the initial dose of TGFβ inhibitor. In some embodiments, MDSC levels may be measured within or at about ten days following administration of the initial dose of TGFβ inhibitor. In certain embodiments, the MDSCs are circulating mMDSCs. In certain embodiments, the MDSCs are circulating gMDSCs.

In certain embodiments, circulating MDSC levels may be used to select, inform treatment, and/or predict response in patients who have not received a checkpoint inhibitor treatment previously. Patients diagnosed with a cancer type with reported high response rates to checkpoint inhibitor therapy (e.g., overall response rate of greater than 30%, greater 40%, greater than 50%, or greater, as reported in the art) who have not received a checkpoint inhibitor therapy previously may be tested to first determine whether their tumors exhibit an immune-excluded or immunosuppressive phenotype. In some embodiments, circulating MDSCs may be used in conjunction with immunohistochemistry, flow cytometry, and/or in vivo imaging methods known in the art to determine the immune phenotype of the tumor. Patients with cancers exhibiting an immune-excluded or immunosuppressive phenotype may be selected to receive a TGFβ inhibitor, such as a TGFβ31-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) and checkpoint inhibitor combination therapy (e.g., an anti-PD1 or anti-PD-L1 antibody). In some embodiments, the circulating MDSCs are circulating mMDSCs. In some embodiments, the circulating MDSCs are circulating gMDSCs. In some embodiments, patients exhibiting an immune-excluded or immunosuppressive phenotype are treated with a TGFβ inhibitor.

In some embodiments, circulating MDSC levels (e.g., circulating mMDSC levels) may be further monitored as an early predictor of treatment response. In certain embodiments, patients diagnosed with a cancer type with reported low response rates to checkpoint inhibitor therapy (e.g., overall response rate of 30% or less, 20% or less, or 10%, or less, as reported in the art) who have not received a checkpoint inhibitor therapy previously may be treated with a combination of a TGFβ inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) and a checkpoint inhibitor therapy. In some embodiments, treatment response in these patients may be predicted by monitoring circulating MDSC levels. In some embodiments, treatment response in these patients may be predicted by monitoring circulating mMDSC levels. In some embodiments, treatment response in these patients may be predicted by monitoring circulating gMDSC levels. In some embodiments, treatment is continued based on the circulating MDSC levels.

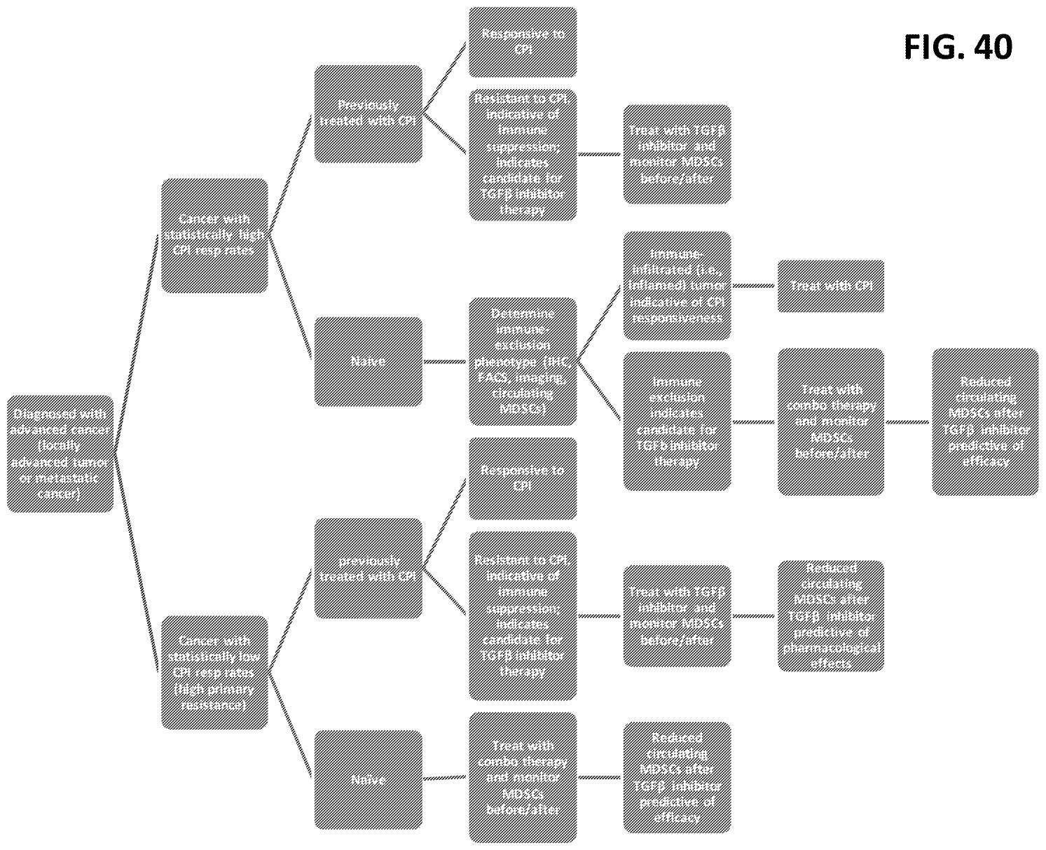

In certain embodiments, circulating MDSC levels may be used for selecting, informing treatment in, and predicting response in patients who are resistant to checkpoint inhibitor therapy or who do not tolerate checkpoint inhibitor therapy (e.g., due to adverse effects). These patients may have primary resistance (i.e., have never shown response to checkpoint inhibitor therapy) or have acquired resistance (i.e., have responded checkpoint inhibitor therapy initially and developed resistance over time). In some embodiments, resistance to checkpoint inhibitor therapy in patients is indicative of immune suppression or exclusion, thus these patients may be selected as candidates for receiving a TGFβ inhibitor therapy, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). In certain embodiments, patients with either primary resistance or acquired resistance to checkpoint inhibitor may be administered a TGFβ inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3), and their response to treatment may be monitored and/or predicted by circulating MDSC levels. In some embodiments, a reduction of at least 10%, at least 15%, at least 20%, at least 25%, or more in circulating MDSC levels may be indicative of response to the TGFβ inhibitor therapy. In some embodiments, a reduction of at least 10%, at least 15%, at least 20%, at least 25%, or more in circulating MDSC levels may indicate pharmacological effects of a treatment, e.g., with a TGFβ inhibitor. In certain embodiments, a decrease in circulating MDSC levels may be indicative of a decrease in tumor size. In certain embodiments, a decrease in circulating mMDSC levels may be indicative of a decrease in tumor size. In certain embodiments, a decrease in circulating gMDSC levels may be indicative of a decrease in tumor size. A chart summarizing exemplary treatment regimens is provided in FIG. 40.

Most TGFβ inhibitors currently in development are not isoform-selective. These include pan-inhibitors of TGFβ, and inhibitors that target TGFβ1/2 and TGFβ1/3. Approaches taken to manage possible toxicities associated with such inhibitors include careful dosing regimens to hit a narrow window in which both efficacy and acceptable safety profiles may be achieved. This may include sparing of an isoform non-selective inhibitor, which may include infrequent dosing and/or reducing dosage per administration. For instance, in lieu of weekly dosing of a biologic TGFβ inhibitor, monthly dosing may be considered. Another example is to dose only in an initial phase of a combination immunotherapy so as to avoid or minimize toxicities associated with TGFβ inhibition.

Because a combination therapy comprising a cancer therapy (such as checkpoint inhibitor therapy) and an isoform-non-selective TGFβ inhibitor may result in a greater risk of toxicity as compared to a TGFβ1-selective inhibitor (e.g. Ab6), in order to mitigate or manage such risk, the isoform-non-selective TGFβ inhibitor may be administered infrequently or intermittently, for example on an “as-needed” basis. In such treatment paradigm, circulating MDSC levels may be monitored periodically in order to determine that the effects of overcoming immunosuppression are sufficiently maintained, so as to ensure antitumor effects of the cancer therapy. During the course of cancer treatment, if MDSCs become elevated, it indicates that the patient benefits from additional doses of a TGFβ inhibitor. Such approach may help reduce unnecessary risk and adverse events associated with TGFβ inhibition, non-isoform-selective inhibitors in particular. In some embodiments, the TGFβ inhibitor targets TGFβ1/2. In some embodiments, the TGFβ inhibitor targets TGFβ1/3. In some embodiments, the TGFβ inhibitor targets TGFβ1/2/3. In some embodiments, the TGFβ inhibitor selectively targets TGFβ1.

Accordingly, the present disclosure provides a TGFβ inhibitor for use in an intermittent dosing regimen for cancer immunotherapy in a patient, wherein the intermittent dosing regimen comprises the following steps: measuring circulating MDSCs (e.g., circulating mMDSCs and/or circulating gMDSCs) in a first sample collected from the patient prior to a TGFβ inhibitor treatment; administering a TGFβ inhibitor to the patient treated with a cancer therapy, wherein the cancer therapy is optionally a checkpoint inhibitor therapy and/or a chemotherapy; measuring circulating MDSCs (e.g., circulating mMDSCs and/or circulating gMDSCs) in a second sample collected from the patient after the TGFβ inhibitor treatment; continuing with the cancer therapy if the second sample shows reduced levels of circulating MDSCs as compared to the first sample. In some embodiments, the intermittent dosing regimen further comprises measuring circulating MDSCs (e.g., circulating mMDSCs and/or circulating gMDSCs) in a third sample; and, administering to the patient an additional dose of a TGFβ inhibitor, if the third sample shows elevated levels of circulating MDSC levels as compared to the second sample. In some embodiments, the TGFβ inhibitor is an isoform-non-selective inhibitor. In some embodiments, the sample is blood or a blood component sample. In some embodiments, the isoform-non-selective inhibitor inhibits TGFβ1/2/3, TGFβ1/2 or TGFβ1/3. Baseline circulating MDSC levels are likely to be elevated in cancer patients as compared to healthy individuals, and subjects with immunosuppressive cancers may have even more elevated circulating MDSC levels. As such, decreases in circulating MDSC levels (e.g., decreases in circulating mMDSC levels) in patients treated with a TGFβ inhibitor therapy such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3), either alone or in combination with a checkpoint inhibitor therapy, may be indicative of a reduction or reversal of immune suppression in the cancer.

In certain embodiments, a TGFβ inhibitor, such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) is administered to a subject with cancer such that the dose of the TGFβ inhibitor is sufficient to reduce or reverse immune suppression in the cancer as indicated by a reduction of circulating MDSC levels (e.g., a reduction of circulating mMDSC levels) and/or a change in the levels of tumor-associated immune cells measured after administering the TGFβ inhibitor treatment as compared to levels measured before administration. In some embodiments, levels of circulating MDSC and/or tumor-associated immune cells are measured before and after administration of a TGFβ inhibitor treatment such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3) in combination with a checkpoint inhibitor therapy, and a reduction of circulating MDSC levels and/or change(s) in the levels of tumor-associated immune cells measured aftertreatment as compared to levels measure before treatment indicates reduction or reversal of immune suppression in the cancer. In some embodiments, a reduction of circulating mMDSC levels and/or change(s) in the levels of tumor-associated immune cells measured after treatment as compared to levels measure before treatment indicates reduction or reversal of immune suppression in the cancer. In some embodiments, a reduction of circulating gMDSC levels and/or change(s) in the levels of tumor-associated immune cells measured after treatment as compared to levels measure before treatment indicates reduction or reversal of immune suppression in the cancer. In some embodiments, treatment is continued in a patient exhibiting a reversal of immune suppression.

Circulating MDSC levels may be determined in a sample such as a whole blood sample or a blood component (e.g., PBMCs). In some embodiments, the sample is fresh whole blood or a blood component of a sample that has not been previously frozen. In certain embodiments, circulating MDSCs may be collected by drawing peripheral blood into heparinized tubes. From peripheral blood, peripheral blood mononuclear cells may be isolated using, e.g., elutriation, magnetic beads separation, or density gradient centrifugation methods (e.g., Ficoll-Paque®) known in the art. In some embodiments, MDSCs may be separated from peripheral blood mononuclear cells by surface marker selection (e.g., using immunofluorescence, e.g., flow cytometry/FACS analysis or immunohistochemistry). G-MDSCs and M-MDSCs may be further distinguished using surface markers provided herein.

### Tumor-Associated Immune Cell Markers

Immune cell markers may be used to determine whether a cancer has an immune-excluded phenotype, and/or may be used in determining treatment efficacy or treatment regimen, alone or in combination with other circulating biomarkers such as circulating MDSCs. If the tumor is determined to have an immune-excluded phenotype, cancer therapy (such as CBT) alone may not be efficacious. Without being bound by theory, the tumor may lack sufficient cytotoxic cells within the tumor environment for effective CBT treatment alone. Thus, an alternative and/or add-on therapy with a TGFβ inhibitor (such as those described herein) may reduce immuno-suppression, thereby providing an improved treatment alone or rendering the resistant tumor more responsive to a cancer therapy. In some embodiments, immune cell markers are measured in biopsies (e.g., core needle biopsies). In some embodiments, patients having an immune-excluded tumor are administered a treatment comprising one or more TGFβ inhibitor (e.g., TGFβ1 inhibitor, e.g., Ab6). In some embodiments, patients having an immune-excluded tumor are administered a treatment comprising one or more TGFβ inhibitor (e.g., TGFβ1 inhibitor, e.g., Ab6) inhibitor and monitored for improvement in condition (e.g., increased immune cell penetration into a tumor, reduced tumor volume, etc.). In some embodiments, a patient exhibiting an improvement in condition after a first round of treatment is administered one or more additional rounds of treatment. In some embodiments, subjects are administered one or more additional treatment in combination with the one or more TGFβ inhibitor (e.g., TGFβ1 inhibitor, e.g., Ab6).

Tumor-associated immune cells that may be used to indicate the immune contexture of a tumor/cancer microenvironment include, but are not limited to, cytotoxic T cells and tumor-associated macrophages (TAMs), as well as tumor-associated MDSCs. Biomarkers to detect cytotoxic T cell levels may include, but are not limited to, the CD8 glycoprotein, granzyme B, perforin, and IFNγ, of which the latter three markers may also be indicative of activated cytotoxic T cells. To measure the level of TAMs, protein markers such as HLA-DR, CD68, CD163, CD206, and other biomarkers, any method known in the art may be used. In certain embodiments, increased levels of cytotoxic T cells, e.g., activated cytotoxic T cells, detected within the tumor microenvironment may be indicative of reduction or reversal of immune suppression. For example, an increase in CD8 expression and perforin, granzyme B, and/or IFNγ expression by tumor-associated immune cells may be indicative of reduction or reversal of immune suppression in the cancer. In certain embodiments, decreased levels of TAMs or tumor-associated MDSCs detected within the tumor microenvironment may be indicative of reduced or reversal of immune suppression. For example, a decrease of HLA-DR, CD68, CD163, and CD206 expression by tumor-associated immune cells may indicate reduced or reversal of immune suppression in the cancer. In certain embodiments, tumor-associated immune cells, e.g., CD8+ T cells, may be used in combination with one or more additional biomarkers to indicate immune contexture of a tumor/cancer microenvironment. In certain embodiments, the immune contexture of a tumor may be characterized by the density, location, organization, and/or functional orientation of tumor-infiltrating immune cells. In certain embodiments, such markers may be used to determine the immune phenotype of a tumor, e.g., to determine if a tumor is immune excluded, inflamed, or desert.

In various embodiments, cytotoxic T cells, e.g., in a patient sample, may be used to determine whether a cancer has an immune-excluded phenotype, and/or may be used in determining treatment efficacy or treatment regimen, alone or in combination with other biomarkers such as circulating MDSCs. For example, CD8 expression and/or the distribution of CD8 expression in a tumor sample may be used. For instance, CD8 expression may be examined in a sample to determine distribution in the tumor (i.e., tumor compartment), stroma (i.e., stroma compartment), and margin (i.e., margin compartment; identified, e.g., by assessing the region approximately 10-100 μm, or 25-75 μm, or 30-60 μm, e.g., 50 μm, between tumor and stroma). In certain embodiments, tumor, stroma, and/or margin compartments within the tumor may be identified using histological methods (e.g., pathologist assessment, pathologist-trained machine learning algorithms, and/or immunohistochemistry). In certain embodiments, CD8+ T cells in a tumor compartment may be referred to as “tumor-associated CD8+ cells”. In certain embodiments, CD8+ T cells in a stroma compartment may be referred to as “stroma-associated CD8+ cells”. In certain embodiments, CD8+ T cells in a margin compartment may be referred to as “margin-associated CD8+ cells”. In some embodiments, CD8 distribution may be determined in a tumor nest (e.g., a mass of cells extending from a common center seen in a cancerous growth), the stroma surrounding the tumor nest, and the margin between the tumor nest and its surrounding stroma (identified, e.g., by assessing the region approximately 10-100 μm, or 25-75 μm, or 30-60 μm, e.g., 50 μm, between the tumor nest and the surrounding stroma).

In certain embodiments, tumor nests may be identified using histological methods (e.g., pathologist assessment, pathologist-trained machine learning algorithms, and/or immunohistochemistry). In certain embodiments, one or more tumor nests may be found within a tumor compartment. In certain embodiments, a tumor may comprise multiple (e.g., at least 5, at least 10, at least 20, at least 25, at least 50, or more) tumor nests.

By default, unless otherwise indicated by context, the term “stroma” or “stroma compartment” refers to the stroma surrounding the tumor, and the term “margin” or “margin compartment” refers to the margin between the tumor and the stroma surround the tumor. In some embodiments, the structural interface between the tumor/tumor nest and the surrounding stroma is determined by imaging analysis. A margin can then be defined as the region surrounding the interface in either direction by a predetermined distance, for example, 10-100 μm. In some embodiments, this distribution may be used prior to administering a TGFβ inhibitor, such as a TGFβ1 inhibitor (e.g., Ab6) to select a patient for treatment and/or predict and/or determine the likelihood of a therapeutic response (e.g., an anti-tumor response) to an anti-cancer therapy comprising an anti-TGFβ inhibitor. For instance, if no or few cytotoxic T cells (e.g., less than 5% CD8+ T cells) are seen in a tumor sample, including in stroma and margin, this may indicate a patient who would not benefit from TGF inhibitor therapy (without being bound by theory, this may be because there are few immune cells to recruit to the tumor). Similarly, if a high density of cytotoxic T cells (e.g., greater than 5% CD8+ T cells) is observed in tumor as well as stroma and margin, this patient may also have limited benefit from TGF inhibitor therapy (without being bound by theory, this may be because immune cells have already infiltrated the tumor).

In contrast, in certain embodiments, the subject's cancer may exhibit an immune-excluded phenotype, in which cytotoxic T cells (e.g., CD8+ T cells) are observed clustered primarily in or near the margin, e.g., at the border between the margin and the tumor, and not significantly infiltrated into the tumor itself (e.g., less than 5% CD8+ T cells in the tumor compartment and greater than 10% CD8+ T cells in the margin and/or stroma compartment). In certain embodiments, the subject's cancer may exhibit an immune-excluded phenotype, in which cytotoxic T cells (e.g., CD8+ T cells) are observed clustered primarily in or near the margin, e.g., at the border between the margin and the tumor (or peri-vasculature), and not significantly infiltrated into the tumor core itself (e.g., less than 5% CD8+ T cells in the tumor compartment and greater than 5% CD8+ T cells in the margin and/or stroma compartment). In certain embodiments, the subject's cancer may exhibit an immune-excluded phenotype, in which cytotoxic T cells (e.g., CD8+ T cells) are observed clustered primarily in or near the margin, e.g., at the border between the margin and the tumor, and not significantly infiltrated into the tumor itself (e.g., less than 5%, less than 10%, less than 15%, or fewer CD8+ T cells in the tumor compartment and greater than 5%, greater than 10%, greater than 15%, or more CD8+ T cells in the margin and/or stroma compartment). In some embodiments, CD8+ content in tumor compartments may be based on any of the methods described in Ziai et al. (PLoS One. 2018; 13(1): e0190158), Massi et al. (J Immunother Cancer. 2019 Nov. 15; 7(1):308), Sharma et al. (Proc Natl Acad Sci USA. 2007 Mar. 6; 104(10):3967-72), or Echarti et al. (Cancers (Basel). 2019 September; 11(9): 1398), the contents of which are hereby incorporated in their entirety. Any of these methods may be used to determine the immune phenotype of the tumor. Tumor samples with this pattern from a patient may indicate a patient likely to benefit from TGF inhibitor therapy (without being bound by theory, this may be because the tumor is actively suppressing the immune response, preventing sufficient ingress of cytotoxic T cells, which could be partially or completely reversed by the TGF inhibitor).

In some embodiments, an immune-excluded phenotype is characterized by determining a cluster score of cytotoxic T cells (e.g., CD8+ T cells) within a tumor-associated compartment, e.g., in the tumor, in the margin near the external perimeters of a tumor mass, and/or in the vicinity of tumor vasculatures. In some embodiments, the cluster score of cytotoxic T cells (e.g., CD8+ T cells) can be determined based on the homogeneity of immune cells in a particular tumor-associated compartment, such that a compartment containing highly uniform distribution of cytotoxic T cells (e.g., CD8+ T cells) yields a high cluster score. In certain embodiments, tumors exhibiting an immune-excluded phenotype may be characterized by lower densities of cytotoxic T cells (e.g., CD8+ T cells) inside the tumor as compared to densities outside of the tumor (e.g., the external perimeters of a tumor mass and/or near the vicinity of vasculatures of a tumor). In some embodiments, the immune-excluded phenotype is characterized by cytotoxic T cells (e.g., CD8+ T cells) in the tumor stroma that are located in close vicinity (e.g., less than 100 μm) to the tumor. In some embodiments, the immune-excluded phenotype is characterized by cytotoxic T cells (e.g., CD8+ T cells) capable of infiltrating the tumor nest and locating at a close distance (e.g., less than 100 μm) to the tumor. In some embodiments, CD8+ T cells can be observed in clusters within a tumor near intratumoral blood vessels as determined for example by endothelial markers. By comparison, upon overcoming immunosuppression by TGF beta inhibitors, more uniform distribution of CD8+ T cells within the tumor can be observed, presumably as a result of the CD8+ cells being able to infiltrate from the perivascular regions and possibly proliferate in the tumor.

In certain embodiments, levels of tumor-infiltrating cytotoxic T cells (e.g., CD8+ T cells) and their activation status may be determined from a tumor biopsy sample obtained from the subject. In some embodiments, tumor biopsy samples, e.g., core needle biopsies, may be obtained at least 28 days priorto and at least 100 days following treatment administration. In some embodiments, tumor biopsy samples, e.g., core needle biopsies, may be obtained about 21 days to about 45 days following treatment administration. In some embodiments, tumor biopsy samples may be obtained via core needle biopsy. In some embodiments, treatment is continued if an increase is detected.

In certain embodiments, the immune phenotype of a subject's cancer may be determined by measuring the cell densities of cytotoxic T cells (e.g., percent of CD8+ T cells per square millimeter or other defined square distance) in a tumor biopsy sample. In certain embodiments, the immune phenotype of a subject's cancer may be determined by comparing the densities of cytotoxic T cells (e.g., CD8+ T cells) inside the tumor to that outside the tumor (e.g., to cells in the margin, e.g., at the external perimeters of a tumor mass and/or near the vicinity of vasculatures of a tumor). In some embodiments, the immune phenotype of a subject's cancer may be determined by comparing the percentage of CD8+ lymphocytes inside the tumor to that outside the tumor. In certain embodiments, the immune phenotype of a subject's cancer may be determined by comparing the cluster or dispersion of cytotoxic T cells (e.g., average number of CD8+ T cells surrounding other CD8+ T cells) in the tumor, stroma, or margin. In certain embodiments, the immune phenotype of a subject's cancer may be determined by measuring the average distance from cytotoxic T cells (e.g., CD8+ T cells) in the stroma to the tumor. In certain embodiments, the immune phenotype of a subject's cancer may be determined by measuring the average depth of cytotoxic T cell (e.g., CD8+ T cell) penetration into the tumor nest. Cell counts and density may be determined using immunostaining and computerized or manual measurement protocols. In certain embodiments, levels of cytotoxic T cells (e.g., CD8+ T cells) may be measured using immunohistochemical analysis of tumor biopsy samples. In certain embodiments, levels of cytotoxic T cells (e.g., CD8+ T cells) may be determined at least 28 days prior to and/or at least 100 days following administering a TGFβ therapy. In certain embodiments, levels of cytotoxic T cells (e.g., CD8+ T cells) may be determined up to about 45 days (e.g., about 21 days to about 45 days) following administering a TGFβ therapy. In some embodiments, levels of cytotoxic T cells (e.g., CD8+ T cells) are determined 5, 10, 15, 20, 25, 30, or more days prior to and/or at least 50, 60, 70, 80, 90, 100, 110, 120, 130, 140, or 150 days following administering a TGFβ therapy (or at any time point in between).

In some embodiments, a tumor with lower levels of cytotoxic T cells (e.g., CD8+ T cells) inside the tumor as compared to cytotoxic T cell levels (e.g., CD8+ T cells) outside the tumor (e.g., the external perimeters of a tumor and/or near the vicinity of vasculatures of a tumor) may be identified as an immune-excluded tumor. In some embodiments, immune-excluded tumors may also have higher levels of cytotoxic T cells (e.g., CD8+ T cells) in the tumor stroma as compared to inside the tumor. In certain embodiments, immune-excluded tumors may be identified by determining the ratio of cytotoxic T cell density (e.g., CD8+ T cells) inside the tumor to outside of the tumor, wherein the ratio is less than 1. In certain embodiments, immune-excluded tumors may be identified by determining the cytotoxic T cell density ratio inside the tumor to density in the tumor margin, wherein the ratio is less than 1. In certain embodiments, immune-excluded tumors may be identified by determining the cell density ratio inside the tumor to density in the tumor stroma, wherein the ratio is less than 1. In certain embodiments, immune-excluded tumors may be identified by comparing the absolute number, percentage, and/or density of cytotoxic T cells (e.g., CD8+ T cells) inside the tumor to outside the tumor (e.g., margin and/or stroma). In some embodiments, the absolute number, percentage, and/or density of cytotoxic T cells (e.g., CD8+ T cells) outside the tumor is at least 2-fold, 3-fold, 4-fold, 5-fold, 7-fold, or 10-fold greater than inside the tumor in an immune-excluded tumor. In some embodiments, an immune-excluded tumor comprises less than 5% CD8+ T cells inside the tumor and greater than 10% CD8+ T cells in the tumor margin and/or stroma. In some embodiments, immune-excluded tumors may be identified by comparing a ratio of compartmentalized cytotoxic T cell density (e.g., density of CD8+ cells inside the tumor to density in the tumor margin and/or stroma) and the ratio of whole tissue cytotoxic T cell density (e.g., CD8+ cells inside the tumor to CD8+ cells in the entire tumor tissue or biopsy), wherein the compartmentalized ratio is greater than the whole tissue ratio. In some embodiments, a tumor with increased cell density of cytotoxic T cells (e.g., CD8+ T cells) at an average distance of about 100 μm or less outside of the tumor may be identified as an immune-excluded tumor. In some embodiments, cytotoxic T cell density (e.g., CD8+ T cells) may be used in conjunction with one or more parameters, such as average CD8+ cluster score. In some embodiments, an average CD8+ clustering score of 50% or less in the tumor indicates immune exclusion.

In some embodiments, a tumor with lower levels of CD8+ T cells inside (e.g., core of) the tumor as compared to CD8+ T cells outside the tumor (e.g., peripheries of the tumor, e.g., the external perimeters of a tumor and/or near the vicinity of vasculatures of a tumor, e.g., in the tumor margin and/or stroma) may be identified as an immune-excluded tumor. In some embodiments, an immune-excluded tumor comprises less than 5%, less than 10%, or less than 15% CD8+ T cells inside the tumor and/or inside one or more tumor nests and greater than 5%, greater than 10%, or greater than 15% CD8+ T cells outside the tumor and/or outside one or more tumor nests. In some embodiments, an immune-excluded tumor comprises less than 5% CD8+ T cells inside the tumor and/or inside one or more tumor nests and greater than 5% CD8+ T cells outside of the tumor and/or outside one or more tumor nests. In some embodiments, an immune-excluded tumor comprises less than 10% CD8+ T cells inside the tumor and/or inside one or more tumor nests and greater than 10% CD8+ T cells outside of the tumor and/or outside one or more tumor nests. In some embodiments, an immune-excluded tumor comprises less than 15% CD8+ T cells inside the tumor and/or inside one or more tumor nests and greater than 15% CD8+ T cells outside of the tumor and/or outside one or more tumor nests.

In some embodiments, a tumor with higher levels of CD8+ T cells inside the tumor as compared to CD8+ T cells outside the tumor (e.g., the external perimeters of a tumor and/or near the vicinity of vasculatures of a tumor, e.g., in the tumor margin and/or stroma) may be identified as an immune-inflamed tumor. In some embodiments, an immune-inflamed tumor comprises greater than 5% CD8+ T cells inside the tumor. In some embodiments, an immune-inflamed tumor comprises greater than 10% CD8+ T cells inside the tumor and/or inside one or more tumor nests. In some embodiments, an immune-inflamed tumor comprises greater than 15% CD8+ T cells inside the tumor and/or inside one or more tumor nests.

In some embodiments, a tumor with low levels of CD8+ T cells both inside and outside the tumor may be identified as an immune desert tumor. In some embodiments, an immune desert tumor comprises less than 5% CD8+ T cells inside the tumor and less than 10% CD8+ T cells in the tumor margin and/or stroma. In some embodiments, an immune desert tumor comprises less than 5% CD8+ T cells inside the tumor (and/or inside one or more tumor nests) and less than 5% CD8+ T cells in the tumor margin and/or stroma.

In some embodiments, CD8+ content in tumor compartments may be determined based on any of the methods described in Ziai et al. (PLoS One. 2018; 13(1): e0190158), Massi et al. (J Immunother Cancer. 2019 Nov. 15; 7(1):308), Sharma et al. (Proc Natl Acad Sci USA. 2007 Mar. 6; 104(10):3967-72), or Echarti et al. (Cancers (Basel). 2019 September; 11(9): 1398), the contents of which are hereby incorporated in their entirety. In some embodiments, any of these methods may be used to determine the immune phenotype of the tumor.

In certain embodiments, the immune phenotype of a subject's cancer may be determined by average percent CD8 positivity (i.e., percentage of CD8+ lymphocytes) as measured over multiple (e.g., at least 5, at least 15, at least 25, at least 50, or more) tumor nests of a tumor (e.g., in one or more tumor biopsy samples). In certain embodiments, the immune phenotype of a given tumor nest may be determined by comparing the CD8 positivity inside the tumor nest to the CD8 positivity outside the tumor nest (e.g., in the tumor nest margin and/or the tumor nest stroma). In certain embodiments, a tumor nest may be identified as immune inflamed if the CD8 positivity inside the tumor nest is greater than 5%. In certain embodiments, a tumor nest may be identified as immune excluded if the CD8 positivity inside the tumor nest is less than 5% and the CD8 positivity in the tumor nest margin is greater than 5%. In certain embodiments, a tumor nest may be identified as an immune desert if the CD8 positivity inside the tumor nest is less than 5% and CD8 positivity in the tumor nest margin is less than 5%. In certain embodiments, a subject's cancer may be identified immune inflamed if greater than 50% of the total tumor area analyzed comprises tumor nests exhibiting immune inflamed phenotype. In certain embodiments, a subject's cancer may be identified as immune excluded if greater than 50% of the total tumor area analyzed comprises tumor nests exhibiting immune excluded phenotype. In certain embodiments, a subject's cancer may be identified as an immune desert if greater than 50% of the total tumor area analyzed comprises tumor nests exhibiting immune desert phenotype. In certain embodiments, a subject's cancer may be identified based on determination of CD8 positivity from more than one sample (e.g., at least three samples, e.g., four samples) taken from the same tumor.

In certain embodiments, tumor biopsy samples may be obtained by core needle biopsy. In certain embodiments, three to five samples (e.g., four samples) may be taken from the same tumor. In certain embodiments, the needle may be inserted along a single trajectory, wherein multiple samples (e.g., three to five samples, e.g., four samples) may be taken at different tumors depths along the same needle trajectory. In certain embodiments, samples taken at different tumor depths may be used to analyze combined CD8 positivity over multiple tumor nests. In certain embodiments, the combined CD8 positivity determined in these samples may be representative of CD8 positivity in the rest of the tumor. In certain embodiments, the combined CD8 positivity determined in these samples may be used to identify immune phenotype of a subject's cancer.

In certain embodiments, the immune phenotype of a subject's tumor may be determined by combined analysis of the absolute number, percentage, ratio, and/or density of CD8+ cells in the tumor and the combined CD8 positivity (i.e., percentage of CD8+ lymphocytes) across tumor nests throughout the tumor.

In certain embodiments, tumor compartments may be identified, determined, and/or analyzed for markers such as CD8 content manually, e.g., by a pathologist inspection of tumor samples. In some embodiments, tumor compartments may be identified, determined, and/or analyzed for markers such as CD8 content by digital analysis, e.g., by using a software or computer program for automated identification. In certain embodiments, a skilled artisan may use such a software or computer program for automated identification of tumor nests and the boundaries between a tumor nest, stroma compartment, and/or tumor margin compartment. In certain embodiments, a software or computer program may be used to evaluate the distribution of suitable markers such as CD8+ T cells in the identified tumor nest, stromal compartment, and/or tumor margin compartment. In certain embodiments, the software or computer program may be based on one or more machine learning algorithms. In certain embodiments, the one or more machine learning algorithms may be based initially on manual classification of reference samples, e.g., by a trained pathologist. In some embodiments, the software or computer program may use a neural network approach with machine learning based on reference samples categorized manually, e.g., by a pathologist. Exemplary softwares or computer programs include any software or computer program that has the capability of intaking an image (e.g., microscope images of a tumor sample comprising immune staining), processing and analyzing the image, and segmenting the tumor compartments in the image based on specific parameters (e.g., nuclear staining, fibroblast staining, CD8+ staining, other biomarkers). In certain embodiments, the softwares or computer program may be any of those provided by Visiopharm, HALO (Indica Labs), CellProfiler Analyst, Aperio Image Analysis, Zeiss ZEN Intellesis, or ImageJ. Such programs may advantageously achieve sufficient resolution for visualizing certain characteristics of individual tumor nests within a solid tumor (e.g., boundaries for tumor nest, stroma, and/or margin compartments), as opposed to analyzing substantially the entire tumor as a whole.

In certain embodiments, a subject whose cancer exhibits an immune-excluded phenotype may be more responsive to a therapy comprising administration of a TGFβ inhibitor (e.g., Ab6). In some embodiments, such a subject is identified for treatment. In some embodiments, such a subject is administered a treatment comprising a TGF inhibitor, such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibodies that bind to αVβ1, αVβ3, αVβ5, αVβ6, αVβ8, α5β1, αIIbβ3, or α8β1 integrins, and inhibit downstream activation of TGFβ. e.g., selective inhibition of TGFβ1 and/or TGFβ3).

In certain embodiments, a subject whose cancer exhibits an immune-excluded phenotype may be more responsive to a combination therapy comprising a TGFβ inhibitor, such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, ligand traps (e.g., TGFβ1/3 inhibitors), and/or an integrin inhibitor (e.g., an antibody that binds to αVβ1, αVβ3, αVβ5, αVβ6, αVβ8, α5β1, αIIbβ3, or a8@1 integrins, and inhibits downstream activation of TGFβ. e.g., selective inhibition of TGFβ1 and/or TGFβ3), and an additional cancer therapy, e.g., a checkpoint inhibitor. In some embodiments, the additional cancer therapy may comprise chemotherapy, radiation therapy (including radiotherapeutic agents), a cancer vaccine, or an immunotherapy comprising a checkpoint inhibitor such as an anti-PD-1, anti-PD-L1, or anti-CTLA-4 antibody. In some embodiments, the checkpoint inhibitor therapy is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (e.g., ABBV-181); and durvalumab (e.g., Imfinzi®). In certain embodiments, a subject whose cancer exhibits an immune-excluded phenotype is administered a combination therapy comprising a TGFβ inhibitor, such as a TGFβ1-selective inhibitor (e.g., Ab6), and an additional cancer therapy, e.g., a checkpoint inhibitor.

In certain embodiments, a subject whose cancer exhibits an immune-excluded phenotype may be more responsive to a combination therapy comprising a TGFβ inhibitor, such as a TGFβ1-selective inhibitor (e.g., Ab6), and a checkpoint inhibitor therapy (e.g., a PD1 or PDL1 antibody). In some embodiments, such a subject is identified for receiving the combination therapy. In some embodiments, such a subject is identified for receiving the combination therapy prior to receiving the checkpoint inhibitor therapy alone. In some embodiments, such a subject is identified for receiving the combination therapy prior to receiving either the checkpoint inhibitor therapy or the TGFβ inhibitor alone. In some embodiments, such a subject is treatment-naïve. In some embodiments, such a subject has previously received a checkpoint inhibitor therapy and is non-responsive to the checkpoint inhibitor therapy. In some embodiments, such a subject has cancer that exhibits an immune-excluded phenotype. In some embodiments, such a subject has previously received a checkpoint inhibitor therapy and is directly given a combination therapy (e.g., bypassing the need to first try treatment with a checkpoint inhibitor alone). In some embodiments, such a subject is administered a combination therapy comprising a TGFβ inhibitor, such as a TGFβ1-selective inhibitor (e.g., Ab6), and an additional cancer therapy, e.g., a PD1 or PDL1 antibody.

In some embodiments, a subject whose cancer exhibits an immune-excluded phenotype may be selected for treatment and/or monitored during and/or after administration of the therapy comprising a TGFβ inhibitor, such as a TGFβ1-selective inhibitor (e.g., Ab6). In some embodiments, patient selection and/or treatment efficacy is determined by measuring the level of cytotoxic T cells (e.g., CD8+ T cells) inside the tumor as compared to the level of cytotoxic T cells (e.g., CD8+ T cells) outside the tumor (e.g., in the margin). In certain embodiments, an increase in the levels of tumor-infiltrating cytotoxic T cells (e.g., CD8+ T cells) inside the tumor relative to outside the tumor (e.g., margin and/or stroma) following administration of a TGFβ inhibitor therapy (e.g., Ab6), alone or in combination with an additional therapy (e.g., a checkpoint inhibitor therapy), may indicate a therapeutic response (e.g., anti-tumor response). For instance, an increase of at least 10%, 15%, 20%, 25%, or more in tumor-infiltrating cytotoxic T cell levels following TGFβ inhibitor treatment (e.g., Ab6) as compared to tumor-infiltrating cytotoxic T cell levels before the treatment may be indicative of therapeutic response (e.g., anti-tumor response). In some embodiments, an increase of at least 10%, 15%, 20%, 25%, or more in total tumor area comprising immune inflamed tumor nests may be indicative of therapeutic response. In some embodiments, levels of cytolytic proteins such as perforin or granzyme B or proinflammatory cytokines such as IFNγ expressed by the tumor-infiltrating cytotoxic T cells may also be measured to determine the activation status of the tumor-infiltrating cytotoxic T cells. In some embodiments, an increase of at least 1.5-fold, or 2-fold, or 5-fold, or more in cytolytic protein levels may be indicative of therapeutic response (e.g., anti-tumor response). In some embodiments, a change of at least a 1.5-fold, 2-fold, 5-fold, or 10-fold, or more increase in IFNγ levels may be indicative of a therapeutic response (e.g., anti-tumor response). In some embodiments, treatment is continued if an increase in tumor-infiltrating cytotoxic T cells (e.g., CD8+ T cells) is detected.

In certain embodiments, a subject whose cancer exhibits an immune-inflamed phenotype may be more responsive to a therapy comprising a checkpoint inhibitor without a TGFβ inhibitor than would a subject having an immune-excluded phenotype. In some embodiments, the checkpoint inhibitor therapy is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (e.g., ABBV-181); and durvalumab (e.g., Imfinzi®). In certain embodiments, a subject whose cancer exhibits an immune-inflamed phenotype is administered a checkpoint inhibitor.

In certain embodiments, immune phenotyping of a subject's tumor may be determined from a tumor biopsy sample (e.g., core needle biopsy sample), for example histologically, using one or more parameters such as, but not limited to, distribution of cytotoxic T cells (e.g., CD8+ T cells), percentage of cytotoxic T cells (e.g., CD8+ T cells) in the tumor versus stromal compartment, and percentage of cytotoxic T cells (e.g., CD8+ T cells) in the tumor margin.

Recognizing that samples collected by a traditional needle biopsy protocol risk inadvertent bias, depending on where within the tumor the needle was inserted, the present disclosure also provides improved methods, where needle biopsy is employed for tumor analysis. According to the present disclosure, the risk of bias inherent to needle biopsy may be significantly reduced by collecting adjacent tumor samples, for example, at least three, but preferably four samples collected from adjacent tumor tissue (e.g., from the same tumor). This may be carried out from a single needle insertion point, by, for example, altering the angle and/or the depth of insertion. Taking into account that some tissue sections prepared from needle biopsy samples may not remain intact during sample processing, and the possibility that a needle may be inserted in the portion of the tumor tissue that does not accurately represent the tumor phenotype, collecting four samples may help mitigate such limitations and provides more representative tumor phenotyping for improved accuracy.

In certain embodiments, a sample may be analyzed for its distribution of cytotoxic T cells (e.g., CD8+ T cells) using a method such as CD8 immunostaining. In certain embodiments, the distribution of cytotoxic T cells (e.g., CD8+ T cells) may be relatively uniform (e.g., distribution is homogeneous throughout the sample, e.g., CD8 density across tumor nests have a variance of 10% or lower). In some embodiments, a tumor nest (or cancer nest) refers to a mass of cells extending from a common center of a cancerous growth. In some embodiments, a tumor nest may comprise cells interspersed in stroma. In certain embodiments, a sample, such as a sample with an even distribution of cytotoxic T cells (e.g., CD8 T cells), may be analyzed to determine the percentages of cytotoxic T cells (e.g., CD8+ T cells) in the tumor and in the stroma. In certain embodiments, a high percentage (e.g., greater than 5%) of cytotoxic T cells (e.g., CD8+ T cells) in the tumor and a low percentage (e.g., less than 5%) of cytotoxic T cells (e.g., CD8+ T cells) in the stroma may be indicative of an inflamed tumor phenotype. In certain embodiments, a low percentage of cytotoxic T cells (e.g., CD8+ T cells) in both the tumor and the stroma (e.g., combined tumor and stroma CD8 percentage of less than 5%) may be indicative of a poorly immunogenic tumor phenotype (e.g., an immune desert phenotype). In certain embodiments, a low percentage (e.g., less than 5%) of cytotoxic T cells (e.g., CD8+ T cell cells) in the tumor and a high percentage (e.g., greater than 5%) of cytotoxic T cells (e.g., CD8+ T cell cells) in the stroma may be indicative of an immune-excluded tumor phenotype. In certain embodiments, a tumor-to-stroma CD8 ratio may be determined by dividing CD8 percentage in the tumor over the percentage in the stroma. In certain embodiments, a tumor-to-stroma CD8 ratio of greater than 1 may be indicative of an inflamed tumor phenotype. In certain embodiments, a tumor-to-stroma CD8 ratio of less than 1 may be indicative of an immune-excluded tumor. In certain embodiments, percentages of cytotoxic T cells may be determined by immunohistochemical analysis of CD8 immunostaining.

In certain embodiments, a sample, such as a sample with uneven distribution of cytotoxic T cells (e.g., CD8 density across tumor nests have a variance of greater than 10%), may be analyzed to determine the margin-to-stroma CD8 ratio. In certain embodiments, such ratio may be calculated by dividing CD8 density in the tumor margin over CD8 density in the tumor stroma. In certain embodiments, an immune excluded tumor exhibits a margin-to-stroma CD8 ratio of greater than 0.5 and less than 1.5.

In certain embodiments, a sample having a margin-to-stroma CD8 ratio of greater than 1.5 may be further analyzed to determine and/or confirm immune phenotyping (e.g., to determine and/or confirm whether the tumor has an immune-excluded phenotype) by evaluating tumor depth. In certain embodiments, tumor depth may be measured in increments of 20 μm-200 μm (e.g., 100 μm). In certain embodiments, tumor depth may be determined by pathological analysis and/or digital image analysis. In certain embodiments, a significant tumor depth may be indicated by a distance of about 2-fold or greater than the depth of the tumor margin. In certain embodiments, a tumor sample may have a tumor margin depth of 100 μm and a tumor depth measurement of greater than 200 μm, such sample would have a tumor depth score of greater than 2, and would therefore have significant tumor depth. In certain embodiments, significant tumor depth may be indicated by a ratio of 2 or greater as determined by dividing tumor depth by the depth of the tumor margin. In certain embodiments, tumor depth may be measured in increments corresponding to the depth of the tumor margin. For instance, the tumor depth of a tumor nest having a tumor margin of 100 μm may be measured in increments of 100 μm. In certain embodiments, a tumor sample with significant tumor depth may exhibit shallow penetration by cytotoxic T cells (e.g., the tumor sample having greater than 5% CD8 T cells but does not exhibit tumor penetration beyond one tumor depth increment). In certain embodiments, a tumor sample with significant tumor depth that exhibits shallow CD8 penetration may be indicative of an immune excluded tumor.

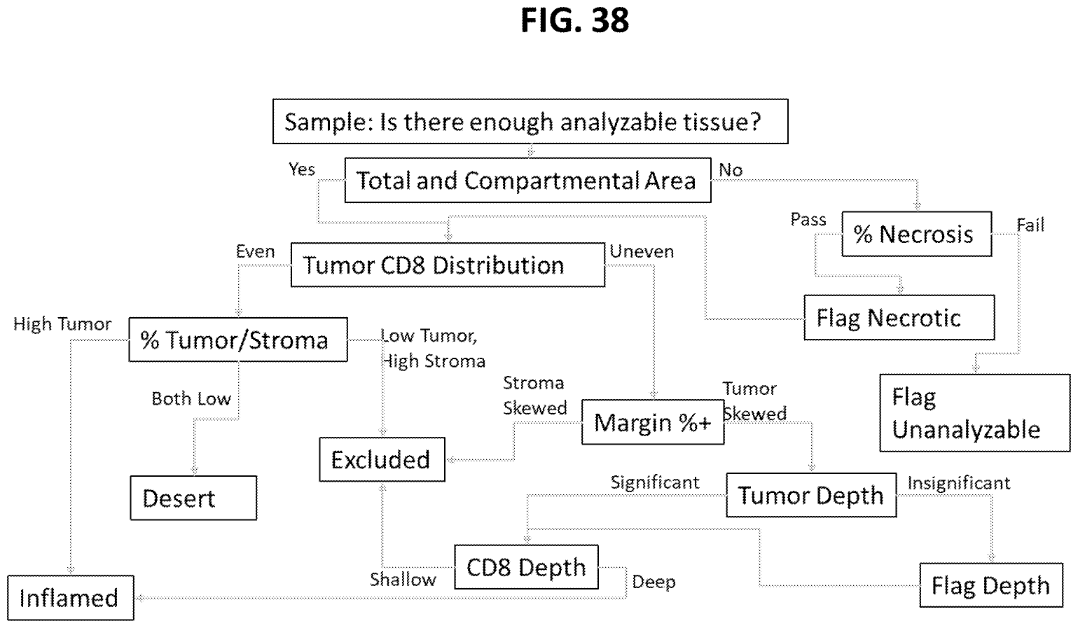

In certain embodiments, a tumor phenotype analysis may be conducted according to any part of the exemplary flow chart shown in FIG. 38, e.g., using all the steps in that figure.

In certain embodiments, a subject whose cancer exhibits an immune excluded phenotype may be selected for TGFβ inhibitor therapy (e.g., a TGFβ1 inhibitor such as Ab6). In certain embodiments, a subject whose cancer exhibits an immune excluded phenotype may be more responsive to a TGFβ inhibitor therapy (e.g., a TGFβ1 inhibitor such as Ab6). In certain embodiments, a subject whose cancer exhibits an immune-excluded phenotype may be more responsive to a combination therapy comprising a TGFβ inhibitor, such as a TGFβ1-selective inhibitor (e.g., Ab6), and a second cancer therapy, e.g., a checkpoint inhibitor therapy (e.g., a PD1 or PDL1 antibody).

In certain embodiments, a response to TGFβ inhibitor therapy (e.g., a TGFβ1 inhibitor such as Ab6) may be monitored and/or determined using parameters such as any of the ones described above. In certain embodiments, a change in a distribution of cytotoxic T cells (e.g., CD8+ T cells) in a pre-treatment tumor sample as compared to a corresponding post-treatment sample from the corresponding tumor may be indicative of a therapeutic response to treatment. In certain embodiments, a change (e.g., increase) of at least 1-fold (e.g., 1.1-fold, 1.2-fold, 1.3-fold, 1.4-fold, 1.5-fold, 1.6-fold, 1.7-fold, 1.8-fold, 1.9-fold, 2-fold, or greater) in the tumor-to-stroma CD8 density ratio between the pre-treatment and post-treatment tumor samples may be indicative of a therapeutic response. In certain embodiments, a change (e.g., increase) of 1.5-fold or greater in the tumor-to-stroma CD8 density ratio between the pre-treatment and post-treatment tumor samples may be indicative of a therapeutic response. In certain embodiments, the tumor-to-stroma CD8 density ratio may be determined by dividing CD8 cell density in the tumor nest over CD8 cell density in the tumor stroma. In certain embodiments, a change (e.g., increase) of 1.5-fold or greater in the density of cytotoxic T cells (e.g., CD8+ T cells) in the tumor margin between the pre-treatment and post-treatment tumor samples may be indicative of a therapeutic response. In certain embodiments, a change (e.g., increase) of 1.5-fold or greater in the tumor depth score of pre-treatment and post-treatment tumor samples may be indicative of a therapeutic response. In some embodiments, the TGFβ inhibitor therapy (e.g., a TGFβ1 inhibitor such as Ab6) achieves at least a 2-fold, e.g., 3-fold, 4-fold, 5-fold, 6-fold, 7-fold, 8-fold, 9-fold, 10-fold, 15-fold, 20-fold, or a greater degree of increase in the number of intratumoral T cells, e.g., when used in conjunction with a checkpoint inhibitor such as a PD-(L)1 antibody, relative to pre-treatment. In certain embodiments, treatment with a TGFβ inhibitor therapy (e.g., a TGFβ1 inhibitor such as Ab6), e.g., alone or in combination with one or more additional cancer therapies, may be continued if a therapeutic response is observed.

In certain embodiments, the pre-treatment and post-treatment samples have comparable tumor depth scores (e.g., variance of less than 0.25 in tumor depth scores of pre-treatment and post-treatment tumor samples) and the samples may be analyzed to determine therapeutic response according to one or more of the parameters described above. In certain embodiments, the pre-treatment and post-treatment samples have comparable total and compartmental areas (e.g., variance of less than 0.25 in analyzable total and compartmental area of pre-treatment and post-treatment tumor samples) and the samples may be analyzed to determine therapeutic response according to one or more of the parameters described above.

In some embodiments, percent necrosis in a tumor sample may be assessed by histological and/or digital image analysis, which may reflect the presence or activities of cytotoxic cells in the tumor. In some embodiments, percent necrosis in tumor samples may be compared in pre-treatment and post-treatment tumor samples collected from a subject administered a TGFβ inhibitor (e.g., Ab6). In some embodiments, increase of greater than 10% in percent necrosis (e.g., the proportion of necrotic area to total tissue area in a tumor sample) between pre-treatment and post-treatment samples may be indicative of a therapeutic response to TGFβ inhibitor therapy, e.g., TGFβ1 inhibitor such as Ab6. In some embodiments, an increase of 10% or greater in percent necrosis in or near the center of the tumor (e.g., the proportion of necrotic area inside the tumor margin) may be indicative of a therapeutic response.

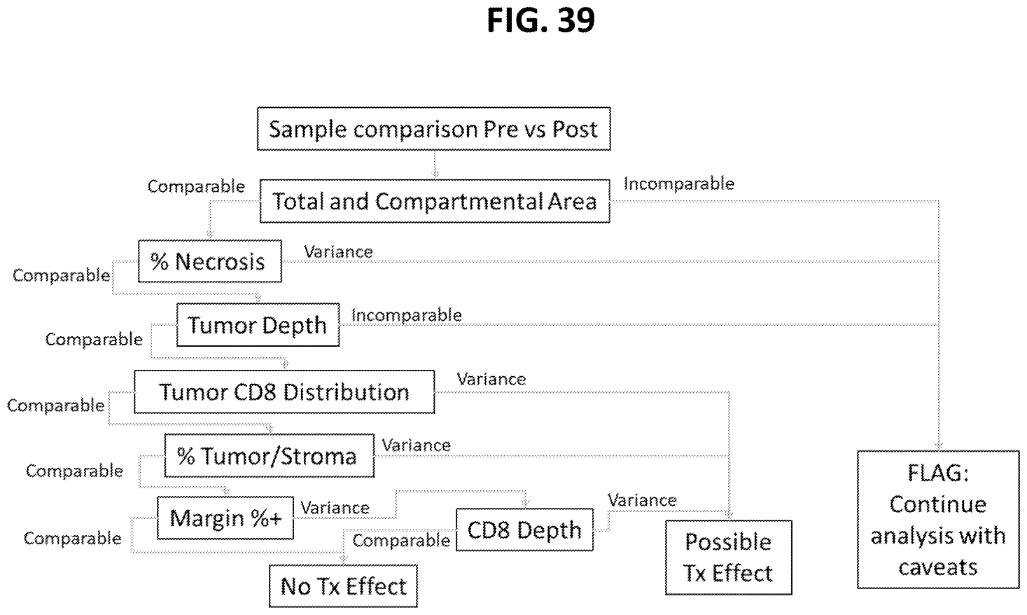

In certain embodiments, a therapeutic response may be determined according to any part of the exemplary flow chart shown in FIG. 39.

In some embodiments, an increased level of tumor-infiltrating cytotoxic T cells (e.g., CD8+ T cells), especially activated cytotoxic T cells, following TGFβ inhibitor therapy (e.g., a TGFβ1 inhibitor such as Ab6) may indicate conversion of an immune-excluded tumor microenvironment toward an immune-infiltrated or “inflamed” microenvironment. For instance, an increase of at least 1%, 2%, 3%, 4%, 5%, 6%, 7%, 8%, 9%, 10%, 15%, 20%, 25%, or more in tumor-associated cytotoxic T cell levels following TGFβ inhibitor treatment (e.g., Ab6) as compared to tumor-associated cytotoxic T cell levels before the treatment may be indicative of a reduction or reversal of immune suppression in the cancer. In some embodiments, an increase of at least 1%, 2%, 3%, 4%, 5%, 6%, 7%, 8%, 9%, 10%, 15%, 20%, 25%, or more in tumor area comprising immune inflamed tumor nests may be indicative of a reduction or reversal of immune suppression in the cancer. In some embodiments, levels of cytolytic proteins such as perforin or granzyme B or proinflammatory cytokines such as IFNγ expressed by the tumor-associated cytotoxic T cells may be measured to determine the activation status of the tumor-associated cytotoxic T cells. In some embodiments, an increase of at least 1-fold, 1.1-fold, 1.2-fold, 1.3-fold, 1.4-fold, 1.5-fold, or 2-fold, or 5-fold, or more in cytolytic protein levels may be indicative of reduction or reversal of immune suppression in the cancer. In some embodiments, a change of at least a 1.5-fold, 2-fold, 5-fold, or 10-fold, or more increase in IFNγ levels may be indicative of a reduction or reversal of immune suppression in the cancer. In some embodiments, treatment with the TGFβ inhibitor therapy (e.g., a TGFβ1 inhibitor such as Ab6) is continued if such a reduction or reversal of immune suppression in the cancer is detected.

Immunosuppressive lymphocytes associated with TMEs include TAMs and MDSCs. A significant fraction of tumor-associated macrophages is of so-called “M2” type, which has an immunosuppressive phenotype. Most of these cells are monocyte-derived cells that originate in the bone marrow. Intratumoral (e.g., tumor-associated) levels of immunosuppressive cells such as TAMs and MDSCs may also be measured to determine the status of immune suppression in a cancer. In some embodiments, a decrease of at least 10%, 15%, 20%, 25%, or more in the level of TAMs may be indicative of reduced or reversal of immune suppression. In certain embodiments, tumor-associated immune cells may be measured from a biopsy sample from the subject prior to and following TGFβ inhibitor treatment (e.g., Ab6). In certain embodiments, biopsy samples may be obtained between 28 days and 130 days following treatment administration.

The concept of “immune contexture” examines the TME from the perspective of tumor-infiltrating lymphocytes (i.e., tumor immune microenvironment or TIME). Tumor immune contexture refers to the localization (e.g., spatial organization) and/or density of the immune infiltrate in the TME. TIME is usually associated with the clinical outcome of cancer patients and has been used for estimating cancer prognosis (see, for example, Fridman et al., (2017) Nat Rev Clin Oncol. 14(12): 717-734) “The immune contexture in cancer prognosis and treatment”). Typically, tissue samples from tumors are collected (e.g., biopsy such as core needle biopsy) for TIL analyses. In some embodiments, TILs are analyzed by FACS-based methods. In some embodiments, TILs are analyzed by immunohistochemical (IHC) methods. In some embodiments, TILs are analyzed by so-called digital pathology (see, for example, Saltz et al., (2018) Cell Reports 23, 181-193. “Spatial organization and molecular correlation of tumor-infiltrating lymphocytes using deep learning on pathology images.”); (Scientific Reports 9: 13341 (2019) “A novel digital score for abundance of tumor infiltrating lymphocytes predicts disease free survival in oral squamous cell carcinoma”). In some embodiments, tumor biopsy samples may be used in various DNA- and/or RNA-based assays (e.g. RNAseq or Nanostring) to evaluate the tumor immune contexture. Without wishing to be bound by theory, it is possible that a reduction or reversal of immune suppression in a cancer/tumor, as indicated by increased cytotoxic T cells and decreased TAMs, may be predictive of therapeutic efficacy in subjects administered with TGFβ inhibitor alone (e.g., Ab6) or in conjunction with a checkpoint inhibitor therapy.

### Circulating/Circulatory Latent-TGFβ

According to the present disclosure, circulating latent TGFβ may serve as a target engagement biomarker. Where an activation inhibitor is selected as a therapeutic candidate, for example, such biomarker may be employed to evaluate or confirm in vivo target engagement by monitoring the levels of circulating TGF beta (circulating TGFβ) before and after administration. In some embodiments, a target engagement marker comprising circulating latent TGFβ (e.g., circulating latent TGFβ1) is measured in a sample. In some embodiments, circulating TGFβ1 in a blood sample (e.g., plasma and/or serum) comprises both latent and mature forms, the former of which representing vast majority of circulatory TGFβ1. In some embodiments, total circulating TGFβ (e.g., total circulating TGFβ1) may be measured, i.e., comprising both latent and mature TGFβ, for example by using an acid treatment step to liberate the mature growth factor (e.g., TGFβ1) from its latent complex and detecting with an enzyme-linked immunosorbent assay (ELISA) assay. In some embodiments, reagents such as antibodies that specifically bind the latent form of TGFβ (e.g., TGFβ1) may be employed to specifically measure circulatory latent TGFβ1. In some embodiments, a majority of the measured circulating TGFβ (e.g., circulating TGFβ1) is released from a latent complex. In some embodiments, the total circulating TGFβ (e.g., circulating TGFβ1) measured is equivalent to dissociated latent TGFβ (e.g., latent TGFβ1) in addition to any free TGFβ (e.g., TGFβ1) present prior to acid treatment, which is known to be only a small fraction of circulating TGFβ1. In some embodiments, only circulating latent TGFβ (e.g., circulating latent TGFβ1) is detectable. In some embodiments, circulating latent TGFβ (e.g., circulating latent circulating TGFβ1) is measured.

In some embodiments, circulating TGFβ (e.g., circulating latent TGFβ1) can be measured from a blood sample by any of the methods described in or adapted from Mussbacher et al., PLos One. 2017 Dec. 8; 12(12):e0188921 and Mancini et al. Transl Res. 2018 February; 192: 15-29, the contents of which are hereby incorporated by reference in their entirety.

Challenges associated with determining blood/serum TGFβ levels with accuracy have been well recognized. Platelets are a main source of TGFβ1 in circulation, and even moderate handling of blood samples, such as blood collection, pipetting of blood samples, mechanical agitation, etc., is known to cause the release of TGFβ1 from platelets in the sample, resulting in skewed readout.

Aspects of the present disclosure include improved assays for measuring circulatory TGFβ levels. Such assays comprise a sample collection step, sample processing step and measuring step.

Sample collection comprises placing a blood sample obtained from a subject (e.g., cancer patient) into a container (e.g., collection tube). Preferably, the collection tube is a sterile, evacuated glass or plastic tube containing anticoagulant. In some embodiments, such tube is about 13 mm times 75 mm in size and has a capacity of about 2.7 mL. In some embodiments, the collection tube contains an anticoagulant solution which includes a form of sodium citrate. In preferred embodiments, the anticoagulant solution is so-called CTAD. The CTAD contains buffered trisodium citrate solution, theophylline, adenosine and dipyrudamole. For example, the CTAD may contain 0.11M buffered trisodium citrate solution (pH about 5.0), 15M theophylline, 3.7M adenosine and 0.198M dipyridamole. Such collection tubes may contain an internal silicone coating to minimize contact activation. Such tubes may be equipped with a closing means (e.g., cap or stopper) aimed to protect users from blood which might splatter when the tube is opened. Such closure may be a rubber stopper, which may be recessed inside the plastic shield, preventing exposure to blood present on the stopper. Examples of commercially available collection tubes include BD Vacutainer™ CTAD Blood Collection Tubes, which is equipped with a Hemogard™ closure. The manufacture's product description suggests that upon collection of blood into the tube, the samples be centrifuged at 1500 g for 15 minutes at room/ambient temperature (18-25° C.). Surprisingly, however, Applicant has found that, contrary to the manufacturer's recommendation, sample collection and processing carried out at 2-8° C. (e.g., about 4° C.) produces superior results for purposes of measuring circulatory TGFβ1 levels.

Accordingly, in some embodiments, circulating TGFβ (e.g., circulating latent TGFβ1) in a blood sample is measured by collecting the blood sample in a collection tube that comprises (containing or coated with) an anticoagulant. In some embodiments, the collection tube comprises a citrate coating. In some embodiments, the collection tube is coated with a solution comprising 0.1-0.5 M buffered trisodium citrate. In some embodiments, the collection tube is coated with a solution comprising 10-20 M theophylline. In some embodiments, the collection tube is coated with a solution comprising 2-5 M adenosine. In some embodiments, the collection tube is coated with a solution comprising 0.1-0.25 M dipyridamole. In some embodiments, the collection tube is coated with a solution having a pH of 4.0-6.0. In some embodiments, the collection tube is coated with an anticoagulant selected from citrate-theophylline-adenosine-dipyridamole (CTAD), citrate (e.g., sodium citrate), acid-citrate-dextrose (ACD), ethylenediaminetetraacetic acid (EDTA), and heparin. In some embodiments, the collection tube is coated with CTAD. In some embodiments, the collection tube is coated with a CTAD solution comprising about 0.11M buffered trisodium citrate solution, about 15 M theophylline, about 3.7 M adenosine, and about 0.198 M dipyridamole. In some embodiments, the CTAD solution has a pH of about 5.0. In some embodiments, the collection tube is glass. In some embodiments, the collection tube has a silicone coating. In some embodiments, the collection tube has a Hemogard™ closure. In some embodiments, the collection tube has a volume capacity of 2-3 mL (e.g., 2.7 mL). In some embodiments, the collection tube is sterile. In some embodiments, the collection tube is a BD Vacutainer™ CTAD blood collection tube (Macey et al. Clin Chem. 2002 June; 48(6 Pt 1):891-9).

Sample processing refers to any handling or processing of a biological sample (e.g., blood sample) following the sample collection step discussed above. The sample processing step may include, for example, centrifugation, fractionation or separation of sample, pipetting, mechanical agitation (e.g., shaking or mixing), etc. In some embodiments, sample processing is carried out to prepare platelet-poor plasma (PPP). A PPP fraction may be prepared from a blood sample for the measurement of circulatory TGFβ1 levels. The term PPP typically refers to blood plasma that contains less than 10,000 platelets per microliter (i.e., <10×10³/μL).

In some embodiments, processing the blood sample comprises incubation and/or centrifugation at a temperature that is lower than room temperature. In some embodiments, processing the blood sample comprises incubation and/or centrifugation at a temperature that is lower than 20° C., lower than 15° C., lower than 10° C., lower than 5° C., or lower. In some embodiments, processing the blood sample comprises incubation and/or centrifugation at 2-8° C. In some embodiments, processing the blood sample comprises incubation and/or centrifugation at about 4° C. In some embodiments, processing the blood sample comprises one or more incubation steps as described in Example 4.

In some embodiments, processing the blood sample comprises one or more centrifugation steps. In some embodiments, processing the blood sample comprises one or more centrifugation steps carried out at about 4° C. In some embodiments, processing the blood sample comprises a centrifugation step at a speed of below 1500×g, below 1000×g, below 800×g, below 400×g, below 250×g, below 200×g, or lower. In some embodiments, processing the blood sample comprises a centrifugation step at a speed of about 150×g. In some embodiments, processing the blood sample comprises a centrifugation step at a speed of above 1500×g, above 2000×g, above 2500×g, above 5000×g, above 7500×g, above 10000×g, above 12000×g, or higher. In some embodiments, processing the blood sample comprises a centrifugation step at a speed of about 2500×g. In some embodiments, processing the blood sample comprises a centrifugation step at a speed of about 12000×g.

In some embodiments, processing the blood sample comprises a first centrifugation step at a speed below 1000×g, and a second centrifugation step at a speed above 2000×g, optionally with one or both steps at about 4° C. In some embodiments, processing the blood sample comprises a first centrifugation step at a speed of about 150×g, and a second centrifugation step at a speed of about 2000×g. In some embodiments, processing the blood sample comprises a first centrifugation step at a speed below 2500×g, and a second centrifugation step at a speed above 10000×g. In some embodiments, processing the blood sample comprises a first centrifugation step at a speed of about 1500×g, and a second centrifugation step at a speed of about 12000×g. In some embodiments, processing the blood sample comprises a first centrifugation step at a speed of between 1000×g to 5000×g, and a second centrifugation step at a speed of between 1000×g to 5000×g. In some embodiments, processing the blood sample comprises a first step and a second centrifugation step, wherein the two centrifugation steps are carried out at the same speed. In some embodiments, processing the blood sample comprises a first centrifugation step at a speed of about 2500×g, and a second centrifugation step at a speed of about 2500×g.

In some embodiments, processing the blood sample comprises one or more centrifugation steps carried out for at least 5 minutes, at least 10 minutes, at least 15 minutes, at least 20 minutes, at least 25 minutes, at least 30 minutes, or longer. In some embodiments, the blood sample is processed by a first centrifugation step for at least 5 minutes, at least 10 minutes, at least 15 minutes, at least 20 minutes, at least 25 minutes, at least 30 minutes, or longer, followed by a second centrifugation step for at least 5 minutes, at least 10 minutes, at least 15 minutes, at least 20 minutes, at least 25 minutes, at least 30 minutes, or longer. In some embodiments, processing the blood sample comprises a first centrifugation step for about 10 minutes, and a second centrifugation step for about 20 minutes. In some embodiments, processing the blood sample comprises a first centrifugation step for about 10 minutes, and a second centrifugation step for about 5 minutes. In some embodiments, processing the blood sample comprises transferring the supernatant portion of the sample to a separate tube after the first centrifugation step, and further processing the supernatant in a second centrifugation step. In some embodiments, the supernatant portion of the sample following the second centrifugation step is used for measuring circulating TGFβ (e.g., circulating latent TGFβ1) levels. In some embodiments, TGFβ (e.g., circulating latent TGFβ1) levels may be determined using Bio-Plex Pro™ TGF-β Assays (Strauss et al. Clin Cancer Res. 2018 Mar. 15; 24(6):1287-1295).

In some embodiments, collection, processing, and/or determination of circulating TGFβ (e.g., circulating latent TGFβ1) levels are conducted at about 4° C.

In some embodiments, processing the blood sample comprises a first centrifugation step of 100-500×g for 5-25 minutes, and a second centrifugation step of 1000-3000×g for 10-40 minutes, each step is optionally carried out at about 4° C. In some embodiments, processing the blood sample comprises a first centrifugation step of 1000-3000×g for 5-25 minutes, and a second centrifugation step of 1000-3000×g for 10-40 minutes, each step is optionally carried out at about 4° C. In some embodiments, processing the blood sample comprises a first centrifugation step of 1000-3000×g for 5-25 minutes, and a second centrifugation step of 5000-15000×g for 2-10 minutes, each step is optionally carried out at about 4° C.

In some embodiments, processing the blood sample comprises a first centrifugation step of 1500×g for 10 minutes, and a second centrifugation step of 12000×g for 5 minutes, optionally with one or both steps carried out at about 4° C. In some embodiments, the blood sample is processed by a first centrifugation step of 2500×g for 10 minutes, followed by a second centrifugation step of 2500×g for 10 minutes, optionally with one or both steps at about 4° C. In some embodiments, one or more additional centrifugation step is applied.

In various embodiments, the present disclosure provides methods of determining and monitoring the level of circulating latent TGFβ in a sample obtained from a patient, such that unwanted or inadvertent TGFβ activation associated with sample processing and preparation is reduced. In certain embodiments, the methods disclosed herein may be used to determine or monitor the level of circulating latent TGFβ1, e.g., by using sample collection methods disclosed herein and/or by normalizing to control markers of platelet activation during collection, e.g., PF4 levels.

Following the sample processing step described above, the resulting samples (e.g., PPP et al.) may be used to carry out one or more measuring steps for circulatory TGFβ. Accordingly, the present disclosure provides, in various embodiments, a method for measuring circulating TGFβ levels in a blood sample, wherein the method comprises a collection step and a processing step, each of which is carried out at 2-8° C. using a CTAD collection tube. The processing step may comprise two centrifugation steps as described above, to generate a PPP fraction from the blood sample. The resulting PPP is used to measure TGFβ levels. In some embodiments, total TGFβ levels, which include both the active and latent TGFβ forms, are measured. In some embodiments, active TGFβ (mature growth factor) levels are measured. In some embodiments, latent TGFβ levels are measured. In some embodiments, a majority of the TGFβ measured in an acidified sample is from circulating latent TGFβ. In some embodiments, the level of the TGFβ1 isoform is selectively measured. In some embodiments, the measuring step may include acidification of the sample to release TGFβ (i.e., mature growth factor) from the latent complex (i.e., proTGFβ, such as proTGFβ1). ELISA-based methods may be employed to then capture and detect/quantitate TGFβ present in the sample.

Such assay steps may be incorporated in a treatment regimen for a patient. For example, such assays may be used for providing information to aid prognosis, diagnosis, target engagement, monitoring therapeutic response, etc.

In some embodiments, circulatory TGFβ levels may serve as a predictive biomarker.

In some embodiments, circulatory TGFβ levels may serve as a predictive biomarker for therapeutic response to a checkpoint inhibitor therapy. In some embodiments, high baseline levels of circulatory TGFβ levels (e.g., in the plasma) may be predictive of poor therapeutic response to a checkpoint inhibitor therapy (e.g., pembrolizumab) (Feun et al. Cancer. 2019 Oct. 15; 125(20):3603-3614).

Accordingly, in various embodiments, the treatment regimen may include administration of a therapy that includes a TGFβ inhibitor, such as TGFβ1 inhibitor. The TGFβ inhibitors include, for example, monoclonal antibodies that bind the latent form of TGFβ (i.e., proTGFβ, such as proTGFβ1) thereby preventing the release of the growth factor, such as Ab6 and other antibodies that work by the same mechanism of action (see, for example, WO 2000/014460, WO 2000/041390, PCT/2021/012930, WO 2018/013939, WO 2020/160291). The TGFβ inhibitors include neutralizing antibodies and engineered constructs that incorporate an antigen-binding fragment thereof. Examples of neutralizing antibodies include GC1008 and its variants, and NIS-793 (XOMA089). The TGFβ inhibitors also include so-called ligand traps, which comprise the ligand binding fragment(s) of the TGFβ receptor(s). Examples of ligand traps include M7824 (bintrafusp alpha) and AVID200. The TGFβ inhibitors also include low molecular weight receptor kinase inhibitors, such as ALK5 inhibitors.

In various embodiments, the patient being administered the treatment regimen is diagnosed with, at risk of developing, or suspected to have a TGFβ-related disease, such as cancer, myeloproliferative disorders (such as myelofibrosis), fibrosis and immune disorders. Thus, in some embodiments, the present disclosure provides a TGFβ inhibitor for use in the treatment of a TGFβ-related disease in a subject, wherein the treatment comprises administration of a composition comprising a TGFβ inhibitor in an amount sufficient to treat the disease, wherein the treatment further comprises determination of circulatory TGFβ levels in accordance with the disclosure herein. In some embodiments, the treatment further comprises determination of circulatory MDSCs. In some embodiments, circulatory MDSC levels are determined by measuring cell-surface marker(s). In some embodiments, the cell-surface marker is LRRC33. In some embodiments, the patient is a cancer patient, wherein optionally the cancer comprises a solid tumor, such as locally advanced or metastatic tumor. In some embodiments, the patient previously received a cancer therapy, wherein the cancer therapy is checkpoint inhibitor, radiation therapy and/or chemotherapy. In some embodiments, the subject was unresponsive or refractory to the cancer therapy, wherein optionally the cancer therapy comprises a checkpoint inhibitor (e.g., checkpoint inhibitor-resistant). In some embodiments, the tumor is refractory to the cancer therapy. In some embodiments, the patient is naïve to a cancer therapy, e.g., a checkpoint inhibitor (i.e., a checkpoint inhibitor-naïve patient). In some embodiments, the checkpoint inhibitor-naïve patient is diagnosed with a type of cancer that has statistically shown to have low response rates (e.g., below 30%, below 25%, below 20%, below 15%, etc.) to checkpoint inhibitors, such as anti-PD-(L)1. In some embodiments, the solid tumor has an immune excluded phenotype. In some embodiments, the solid tumor has low expression of PD-L1.

In various embodiments, the present disclosure provides methods of treating a TGFβ-related disorder, comprising monitoring the level of circulating TGFβ, e.g., circulating latent TGFβ (e.g., TGFβ1) in a sample obtained from a patient (e.g., in the blood, e.g., plasma and/or serum, of a patient) receiving a TGFβ inhibitor. In certain embodiments, circulating TGFβ, e.g., circulating latent TGFβ (e.g., TGFβ1) may be measured in plasma samples collected from the subject. In certain embodiments, measuring TGFβ, e.g., circulating latent TGFβ (e.g., TGFβ1) from the plasma may reduce the risk of inadvertently activating TGFβ, such as that observed during serum preparations and/or processing. Accordingly, the present disclosure includes a TGFβ inhibitor for use in the treatment of diseases such as cancer, myelofibrosis, and fibrosis, in a subject, wherein the treatment comprises a step of measuring circulating TGFβ levels from a plasma sample collected from the subject. Such samples may be collected before and/or after administration of a TGFβ inhibitor to treat such diseases.

The level of circulating TGFβ (e.g., circulating latent TGFβ1) may be monitored alone or in conjunction with one or more of the biomarkers disclosed herein (e.g., MDSCs). In certain embodiments, circulating TGFβ (e.g., circulating latent TGFβ1) may be monitored alone or in conjunction with one or more of total platelet count, phosphorylated Smad2 level, and/or treatment duration. In certain embodiments, the TGFβ inhibitor may be administered alone or in conjunction with an additional cancer therapy. In some embodiments, the treatment may be administered to a subject afflicted with a TGFβ-related cancer or myeloproliferative disorder. In some embodiments, the TGFβ inhibitor is a TGFβ1-selective antibody or antigen-binding fragment thereof encompassed in the current disclosure (e.g., Ab6). In some embodiments, the TGFβ inhibitor is an isoform-non-selective TGFβ inhibitor (such as low molecular weight ALK5 antagonists, neutralizing antibodies that bind two or more of TGFβ1/2/3, e.g., GC1008 and variants, antibodies that bind TGFβ1/3, and ligand traps, e.g., TGFβ1/3 inhibitors). In some embodiments, the TGFβ inhibitor is an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). In some embodiments, the additional cancer therapy may comprise chemotherapy, radiation therapy (including radiotherapeutic agents), a cancer vaccine, or an immunotherapy, such as a checkpoint inhibitor therapy, e.g., an anti-PD-1, anti-PD-L1, or anti-CTLA-4 antibody. In some embodiments, the checkpoint inhibitor therapy is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181); and durvalumab (e.g., Imfinzi®).

In various embodiments, circulating TGFβ (e.g., circulating latent TGFβ1) may be measured in a sample obtained from a subject (e.g., whole blood or a blood component). In various embodiments, the circulating latent TGFβ levels (e.g., latent TGFβ1) may be measured within 1, 2, 3, 4, 5, 6, 7, 8, 10, 12, 14, 16, 18, 21, 22, 25, 28, 30, 35, 40, 45, 48, 50, or 56 days following administration of the TGFβ inhibitor to a subject, e.g., up to 56 days after administration of a therapeutic dose of a TGFβ inhibitor. In various embodiments, the circulating latent TGFβ levels (e.g., circulating latent TGFβ1) may be measured about 8 to about 672 hours following administration of a therapeutic dose of a TGFβ inhibitor. In various embodiments, the circulating latent TGFβ levels (e.g., circulating latent TGFβ1) may be measured about 72 to about 240 hours (e.g., about 72 to about 168 hours, about 84 to about 156 hours, about 96 to about 144 hours, about 108 to about 132 hours) following administration of a therapeutic dose of a TGFβ inhibitor. In various embodiments, the circulating latent TGFβ levels (e.g., circulating latent TGFβ1) may be measured about 120 hours following administration of a therapeutic dose of a TGFβ inhibitor. In some embodiments, the circulating latent TGFβ levels (e.g., circulating latent TGFβ1) may be measured by any method known in the art (e.g., ELISA). In preferred embodiments, circulating TGFβ levels are measured from a blood sample (e.g., a plasma sample).

In various embodiments, the present disclosure encompasses a method of treating cancer in a subject, wherein the treatment comprises determining a level of circulating TGFβ in the subject prior to administering a TGFβ inhibitor, administering to the subject a therapeutically effective amount of the TGFβ inhibitor, and determining a level of circulating TGFβ in the subject after administration. In some embodiments, the circulating TGFβ level is determined or has been determined by processing a blood sample from the subject below room temperature in a sample tube coated with an anticoagulant.

In various embodiments, a method of treating a cancer or other TGF-related disorder comprises administering a TGFβ inhibitor (e.g., an anti-TGFβ1 antibody) to a patient in need thereof and confirming the level of target engagement by the inhibitor. In some embodiments, determining the level of target engagement comprises determining the levels of circulating TGFβ (e.g., circulating latent TGFβ1) in a sample obtained from a patient (e.g., in the blood or a blood component of a patient) receiving the TGFβ inhibitor. In some embodiments, an increase in circulating TGFβ (e.g., circulating latent TGFβ1) after administration of the TGF inhibitor indicates target engagement. In some embodiments, the present disclosure provides a method of determining targeting engagement in a subject having cancer, comprising determining a level of circulating TGFβ (e.g., circulating latent TGFβ1) in the subject prior to administering a TGFβ inhibitor, administering to the subject a therapeutically effective amount of the TGFβ inhibitor, and determining a level of circulating TGFβ in the subject after administration. In some embodiments, an increase in circulating TGFβ levels (e.g., circulating latent TGFβ1 levels) after administration as compared to before administration indicates target engagement of the TGFβ inhibitor. In some embodiments, an increase in circulating TGFβ (e.g., circulating latent TGFβ1) after administration of the TGFβ inhibitor indicates target engagement, wherein the increase is at least 1.5-fold, at least 2-fold, at least 2.5-fold, at least 3-fold, at least 4-fold, at least 5-fold, at least 6-fold, at least 7-fold, at least 8-fold, at least 9-fold, at least 10-fold, or more over baseline level. In some embodiments, the circulating TGFβ levels are determined or have been determined by processing a blood sample from the subject below room temperature in a sample tube coated with an anticoagulant. In some embodiments, further therapeutically effective amount of the TGFβ inhibitor are administered if target engagement is detected.

In various embodiments, the present disclosure also provides methods of using circulating TGFβ levels (e.g., circulating latent TGFβ1 levels) to predict therapeutic response, as well as for informing further treatment decisions (e.g., by continuing treatment if an increase is observed). In some embodiments, an additional dose of the TGFβ inhibitor (e.g., an anti-TGFβ1 antibody) is administered if target engagement is detected. In some embodiments, the method of determining therapeutic efficacy comprises determining a level of circulating TGFβ in the subject prior to administering a TGFβ inhibitor, administering to the subject a therapeutically effective amount of the TGFβ inhibitor, and determining a level of circulating TGFβ in the subject after administration. In preferred embodiments, circulating TGFβ levels are measured from a blood sample. In some embodiments, the circulating TGFβ levels are determined or have been determined by processing the blood sample from the subject below room temperature in a sample tube coated with an anticoagulant. In some embodiments, further therapeutically effective amount of the TGFβ inhibitor are administered if efficacy is detected.

In one aspect of the current disclosure, levels of circulating TGFβ (e.g., circulating latent TGFβ1) are determined to inform treatment and predict therapeutic efficacy in subjects administered a TGFβ inhibitor such as a TGFβ1-selective inhibitor described herein. In certain embodiments, a TGFβ inhibitor (e.g., Ab6) is administered alone or concurrently (e.g., simultaneously), separately, or sequentially with an additional cancer therapy, e.g., a checkpoint inhibitor therapy, such that the amount of TGFβ1 inhibition administered is sufficient to increase the levels of circulating TGFβ (e.g., circulating latent TGFβ1) as compared to baseline levels. Circulating TGFβ levels (e.g., circulating latent TGFβ1) may be measured prior to or after each treatment such that an increase in circulating latent-TGFβ levels (e.g., latent TGFβ1) following the treatment indicates therapeutic efficacy. For instance, circulating TGFβ levels (e.g., circulating latent TGFβ1) may be measured prior to and after the administration of a TGFβ inhibitor (e.g., Ab6) and an increase in circulating TGFβ levels (e.g., latent TGFβ1) following the treatment predicts therapeutic efficacy. In some embodiments, treatment is continued if an increase in circulating TGFβ is detected. In certain embodiments, circulating TGFβ levels (e.g., circulating latent TGFβ1) may be measured prior to and following administration of a first dose of a TGFβ inhibitor such as a TGFβ1 inhibitor described herein (e.g., Ab6), and an increase in circulating TGFβ levels (e.g., circulating latent TGFβ1) following the administration predicts therapeutic efficacy and further warrants administration of a second or more dose(s) of the TGFβ inhibitor. In some embodiments, circulating TGFβ levels (e.g., circulating latent TGFβ1) may be measured prior to and after a combination treatment of TGFβ inhibitor such as a TGFβ1-selective inhibitor (e.g., Ab6), and an additional therapy (e.g., a checkpoint inhibitor therapy), administered concurrently (e.g., simultaneously), separately, or sequentially, and a change in circulating latent-TGFβ levels following the treatment predicts therapeutic efficacy. In some embodiments, treatment is continued if an increase is detected. In some embodiments, an increase in circulating TGFβ (e.g., circulating latent TGFβ1) after administration of the TGFβ inhibitor indicates therapeutic efficacy, wherein the increase is at least 1.5-fold, at least 2-fold, at least 2.5-fold, at least 3-fold, at least 4-fold, at least 5-fold, at least 6-fold, at least 7-fold, at least 8-fold, at least 9-fold, at least 10-fold, or more, as compared to baseline. In some embodiments, the increase in circulating TGFβ levels (e.g., circulating latent TGFβ1) following a combination treatment may warrant continuation of treatment. In preferred embodiments, circulating TGFβ levels are measured from a blood sample, wherein the blood sample is optionally processed below room temperature in a sample tube coated with an anticoagulant.

In certain embodiments, the current disclosure provides a method of treating a cancer in a subject, comprising administering a second dose of a TGFβ inhibitor to a subject having an elevated level of circulating TGFβ after receiving a first dose the TGFβ inhibitor, wherein the level of TGFβ has been measured by processing a blood sample from the subject below room temperature in a sample tube coated with an anticoagulant.

In certain embodiments, the current disclosure provides a method of treating a cancer in a subject comprising determining a level of circulating TGFβ in the subject prior to administering a TGFβ inhibitor, administering to the subject a first dose of TGFβ inhibitor, determining a level of circulating TGFβ in the subject after administration, and administering a second dose of the TGFβ inhibitor to the subject if the level of circulating TGFβ is elevated. In some embodiments, measuring the level of circulating TGFβ comprises processing a blood sample from the subject below room temperature in a sample tube coated with an anticoagulant. In some embodiments, the level of circulating TGFβ after the first dose of the TGFβ inhibitor is elevated by at least 1.5-fold, at least 2-fold, at least 2.5-fold, at least 3-fold, at least 4-fold, at least 5-fold, or more as compared to baseline (e.g., the level of circulating TGFβ before the first dose of the TGFβ inhibitor). In some embodiments, the circulating TGFβ is latent TGFβ. In some embodiments, the circulating TGFβ is circulating TGFβ1.

In some embodiments, the cancer comprises a solid tumor, wherein optionally the solid tumor is selected from: melanoma (e.g., metastatic melanoma), triple-negative breast cancer, HER2-positive breast cancer, colorectal cancer (e.g., microsatellite stable-colorectal cancer), lung cancer (e.g., metastatic non-small cell lung cancer, small cell lung cancer), esophageal cancer, pancreatic cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer (e.g., gastric cancer), head and neck squamous cell cancer, urothelial carcinoma (e.g., metastatic urothelial carcinoma), hepatocellular carcinoma, or thyroid cancer.

In various embodiments, the current disclosure encompasses a method of treating a TGFβ-related disorder comprising administering a therapeutically effective amount of a TGFβ inhibitor to a subject having a TGFβ-related disorder, wherein the therapeutically effective amount is an amount sufficient to increase the level of circulating TGFβ (e.g., circulating latent TGFβ1). In certain embodiments, the TGFβ inhibitor is a TGFβ activation inhibitor. In certain embodiments, the TGFβ inhibitor is a TGFβ1 inhibitor (e.g., Ab6). In certain embodiments, the circulating TGFβ is latent TGFβ1. In some embodiments, the therapeutically effective amount of the TGFβ inhibitor (e.g., Ab6) is between 0.1-30 mg/kg per dose. In some embodiments, therapeutically effective amount of the TGFβ inhibitor (e.g., Ab6) is between 1-30 mg/kg per dose. In some embodiments, the therapeutically effective amount of the TGFβ inhibitor (e.g., Ab6) is between 5-20 mg/kg per dose. In some embodiments, the therapeutically effective amount of the TGFβ inhibitor (e.g., Ab6) is between 3-10 mg/kg per dose. In some embodiments, the therapeutically effective amount of the TGFβ inhibitor (e.g., Ab6) is between 1-10 mg/kg per dose. In some embodiments, the therapeutically effective amount of the TGFβ inhibitor (e.g., Ab6) is between 2-7 mg/kg per dose. In some embodiments, the therapeutically effective amount of the TGFβ inhibitor (e.g., Ab6) is about 2-6 mg/kg per dose. In some embodiments, the therapeutically effective amount of the TGFβ inhibitor (e.g., Ab6) is about 1 mg/kg per dose. In some embodiments, doses are administered about every three weeks. In some embodiments, the TGFβ inhibitor (e.g., Ab6) is dosed weekly, every 2 weeks, every 3 weeks, every 4 weeks, monthly, every 6 weeks, every 8 weeks, bi-monthly, every 10 weeks, every 12 weeks, every 3 months, every 4 months, every 6 months, every 8 months, every 10 months, or once a year. In preferred embodiments, circulating TGFβ levels are measured from a blood sample (e.g., a plasma sample, serum sample, etc.).

In various embodiments, total circulatory TGFβ1 (e.g., circulating latent TGFβ1) in blood samples collected from patients may range between about 2-200 ng/mL at baseline, although the measured amounts vary depending on the individuals, health status, and the exact assays being employed. In certain embodiments, total circulatory TGFβ1 (e.g., circulating latent TGFβ1) in blood samples collected from patients may range between about 1 ng/mL to about 10 ng (e.g., about 1000 μg/mL to about 7000 μg/mL). In certain embodiments, the level of circulating latent TGFβ (e.g., latent TGFβ1) following administration of a TGFβ inhibitor (e.g., Ab6) is increased by at least 1.5-fold (e.g., at least 1.5-fold, at least 2-fold, at least 3-fold, at least 4-fold, at least 5-fold, at least 6-fold, at least 7-fold, at least 8-fold, at least 9-fold, at least 10-fold, or more) as compared to circulating latent TGFβ levels prior to the administration (e.g., baseline). In preferred embodiments, circulating TGFβ levels are measured from a blood sample (e.g., a plasma sample, serum sample, etc.).

In certain embodiments, circulating TGFβ levels (e.g., circulating latent TGFβ1) may be used to monitor target engagement and pharmacological activity of a TGFβ inhibitor in a subject receiving a TGFβ inhibitor therapy (e.g., a TGFβ activation inhibitor, e.g., Ab6). In certain embodiments, circulating TGFβ levels (e.g., circulating latent TGFβ1 levels) may be measured prior to and after administration of a first dose of TGFβ inhibitor (e.g., Ab6) such that an increase of at least 1.5-fold (e.g. at least 1.5-fold, at least 2-fold, at least 3-fold, at least 4-fold, at least 5-fold, at least 6-fold, at least 7-fold, at least 8-fold, at least 9-fold, at least 10-fold, or more) over baseline in circulating latent TGFβ levels following the administration indicates target engagement (e.g., binding of the TGFβ inhibitor to human large latent proTGFβ1 complex). In certain embodiments, circulating TGFβ levels (e.g., circulating latent TGFβ1) may be measured prior to and after administration of a first dose of TGFβ inhibitor (e.g., Ab6) such that an increase in circulating TGFβ levels (e.g., circulating latent TGFβ1) following the administration indicates therapeutic efficacy. In certain embodiments, treatment is continued if an increase in circulating TGFβ levels (e.g., circulating latent TGFβ1) following administration of a TGFβ inhibitor (e.g., Ab6) is detected. In preferred embodiments, circulating TGFβ levels (e.g., circulating latent TGFβ1) are measured from a blood sample (e.g., a plasma sample, serum sample, etc.).

In some embodiments, circulating TGFβ levels (e.g., circulating latent TGFβ1) may be measured prior to and after administration of a first dose of a TGFβ inhibitor (e.g., Ab6), and an increase in circulating TGFβ levels (e.g., circulating latent TGFβ1) after the administration indicates target engagement, treatment response, and/or further warrants administration of a second or more dose(s) of the TGFβ inhibitor. In another embodiment, circulating TGFβ levels (e.g., circulating latent TGFβ1) may be measured prior to and after administration of a first dose of a combination treatment comprising a checkpoint inhibitor therapy and a TGFβ inhibitor such as a TGFβ1-selective inhibitor (e.g., Ab6), and an increase in circulating TGFβ levels (e.g., circulating latent TGFβ1) after the administration indicates target engagement, treatment response, and/or further warrants continuation of treatment. In various embodiments, the combination therapy comprises a checkpoint inhibitor therapy and a TGFβ inhibitor such as a TGFβ1-selective inhibitor (e.g., Ab6), an isoform-non-selective inhibitor (e.g., low molecular weight ALK5 antagonists), neutralizing antibodies that bind two or more of TGFβ1/2/3 (e.g., GC1008 and variants), antibodies that bind TGFβ1/3, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). In preferred embodiments, circulating TGFβ levels are measured from a blood (e.g., plasma sample, serum sample, etc.).

In any of the preceding embodiments, circulating TGFβ can be circulating TGFβ1 or circulating latent TGFβ1. In various embodiments, the circulating TGFβ1 or circulating latent TGFβ1 is measured from a blood sample collected from the subject. In various embodiments, the blood sample is processed below room temperature in a sample tube containing or coated with an anticoagulant.

### Smad2 Phosphorylation

According to the present disclosure, activation of Smad2 may serve as a marker for target engagement and/or therapeutic efficacy. In certain embodiments, Smad2 activation is detected by measuring a level of Smad2 phosphorylation (p-Smad2) and/or p-Smad2 nuclear translocation. In certain embodiments, p-Smad2 levels and/or p-Smad2 nuclear translocation is measured by immunohistochemistry. In certain embodiments, p-Smad2 nuclear translocation is determined by nuclear masking analysis.

In certain embodiments, a method for determining therapeutic efficacy in a subject being treated for cancer comprises determining a level of p-Smad2 nuclear translocation in a tumor sample obtained from the subject prior to administering a therapy comprising a TGFβ inhibitor; administering to the subject one or more doses of the TGFβ inhibitor; and determining a level of p-Smad2 nuclear translocation in a tumor sample obtained from the subject after the administration; wherein a decrease in p-Smad2 nuclear translocation after the administration as compared to before the administration indicates therapeutic efficacy. In certain embodiments, the p-Smad2 nuclear translocation after the administration is decreased by at least 1.3-fold, at least 1.5-fold, at least 2-fold, at least 3-fold, at least 4-fold, at least 5-fold, or more as compared to the p-Smad2 nuclear translocation before the administration. In certain embodiments, one or more additional doses of the treatment is administered if a decrease in p-Smad2 nuclear translocation is observed.

In certain embodiments, a method for determining target engagement in a subject having cancer comprises determining a level of p-Smad2 nuclear translocation in a tumor sample obtained from the subject prior to administering a therapy comprising a TGFβ inhibitor; administering to the subject one or more doses of the TGFβ inhibitor; determining a level of p-Smad2 nuclear translocation in a tumor sample obtained from the subject after the administration; and wherein a decrease in p-Smad2 nuclear translocation after the administration as compared to before the administration indicates target engagement of the TGFβ inhibitor. In certain embodiments, the p-Smad2 nuclear translocation after the administration is decreased by at least 1.3-fold, at least 1.5-fold, at least 2-fold, at least 3-fold, at least 4-fold, at least 5-fold, or more as compared to the p-Smad2 nuclear translocation before the administration. In certain embodiments, one or more additional doses of the treatment is administered if a decrease in p-Smad2 nuclear translocation is observed.

In certain embodiments, a method for treating cancer in a subject or a TGFβ inhibitor for use in treating cancer, wherein the method or the TGFβ inhibitor for use comprises determining a level of p-Smad2 nuclear translocation in a tumor sample obtained from the subject prior to administering a therapy comprising a TGFβ inhibitor; administering to the subject a first dose of the TGFβ inhibitor; determining a level of p-Smad2 nuclear translocation in a tumor sample obtained from the subject after the administration; and administering to the subject one or more additional doses of the TGFβ inhibitor if the p-Smad2 nuclear translocation after the administration of the first dose is decreased as compared to the p-Smad2 nuclear translocation before the administration of the first dose. In certain embodiments, the p-Smad2 nuclear translocation after the administration is decreased by at least 1.3-fold, at least 1.5-fold, at least 2-fold, at least 3-fold, at least 4-fold, at least 5-fold, or more as compared to the p-Smad2 nuclear translocation before the administration. In certain embodiments, the treatment comprises administering one or more additional doses of the treatment if a decrease in p-Smad2 nuclear translocation is observed.

In any of the preceding embodiments, the TGFβ inhibitor is a TGFβ1 inhibitor, e.g., Ab6.

### Immune Safety

Cytokines play an important role in normal immune responses, but when the immune system is triggered to become hyperactive, the positive feedback loop of cytokine production can lead to a “cytokine storm” or hypercytokinemia, a situation in which excessive cytokine production causes an immune response that can damage organs, especially the lungs and kidneys, and even lead to death. Such condition is characterized by markedly elevated proinflammatory cytokines in the serum. Historically, a Phase 1 Trial of the anti-CD28 monoclonal antibody TGN1412 in healthy volunteers led to a life-threatening “cytokine storm” response resulted from an unexpected systemic and rapid induction of proinflammatory cytokines (Suntharalingam G et al., N Engl J Med. 2006 Sep. 7; 355(10):1018-28). This incident prompted heightened awareness of the potential danger associated with pharmacologic stimulation of T cells.

Whilst TGFβ-directed therapies do not target a specific T cell receptor or its ligand, Applicant of the present disclosure reasoned that it was prudent to carry out immune safety assessment, including, for example, in vitro cytokine release assays, in vivo cytokine measurements from plasma samples of non-human primate treated with a TGFβ inhibitor, and platelet assays using human platelets. Exemplary assays were previously described, for instance in PCT/US2021/012969 at Example 23.

In some embodiments, one or more of the cytokines IL-2, TNFα, IFNγ, IL-1P, CCL2 (MCP-1), and IL-6 may be assayed, e.g., by exposure to peripheral blood mononuclear cell (PBMC) constituents from heathy donors. Cytokine response after exposure to an antibody disclosed herein, e.g., Ab6, may be compared to release after exposure to a control, e.g., an IgG isotype negative control antibody. Cytokine activation may be assessed in plate-bound and/or soluble assay formats. Levels of IFNγ, IL-2, IL-1p, TNFα, IL-6, and CCL2 (MCP-1) should not exceed 10-fold, e.g., 8-, 6-, 4-, or 2-fold the activation in the negative control. In some embodiments, a positive control may also be used to confirm cytokine activation in the sample, e.g., in the PBMCs. In some embodiments, these in vitro cytokine release results may be further confirmed in vivo, e.g., in an animal model such as a monkey toxicology study, e.g., a 4-week GLP repeat-dose monkey study as described in PCT/US2021/012969 at Example 24.

Human platelets have been reported to express GARP, which can form TGFβ1 LLCs (Tran et al., 2009. Proc Natl Acad Sci USA. 106(32): 13445-13450). In some embodiments, an antibody disclosed herein, e.g., Ab6, does not significantly bind to and/or activate platelets. In some embodiments, platelet activation is evaluated in vitro, as described in Example 23. In some embodiments, platelet aggregation, binding, and activation may be assessed in human whole blood or platelet-rich plasma from healthy donors. Platelet aggregation and binding after exposure to an antibody disclosed herein, e.g., Ab6 may be compared to exposure to a negative control, e.g., saline solution, or a reference sample, e.g., a buffered solution. In certain embodiments, platelet aggregation and binding do not exceed 10% above the aggregation in the negative control. In some embodiments, platelet activation following exposure to an antibody disclosed herein, e.g., Ab6, may be compared to exposure to a positive control, e.g., adenosine diphosphate (ADP). The activation status of platelets may be determined by surface expression of activation markers e.g., CD62P (P-Selectin) and GARP detectable by flow cytometry. Platelet activation should not exceed 10% above the activation in the negative control. In some embodiments, in vitro platelet response results may be further confirmed in vivo, e.g., in an animal model such as a monkey toxicology study, e.g., a 4-week GLP repeat-dose monkey study.

In some embodiments, selection of an antibody or an antigen-binding fragment thereof for therapeutic use may include: identifying an antibody or antigen-binding fragment that meets the criteria of one or more of those described herein; carrying out an in vivo efficacy study in a suitable preclinical model to determine an effective amount of the antibody or the fragment; carrying out an in vivo safety/toxicology study in a suitable model to determine an amount of the antibody that is safe or toxic (e.g., MTD, NOAEL, or any art-recognized parameters for evaluating safety/toxicity); and, selecting the antibody or the fragment that provides at least a three-fold therapeutic window (preferably 6-fold, more preferably a 10-fold therapeutic window, even more preferably a 15-fold therapeutic window). In certain embodiments, the in vivo efficacy study is carried out in two or more suitable preclinical models that recapitulate human conditions. In some embodiments, such preclinical models comprise a TGFβ1-positive cancer, which may optionally comprise an immunosuppressive tumor. The immunosuppressive tumor may be resistant to a cancer therapy such as CBT, chemotherapy and radiation therapy (including a radiotherapeutic agent). In some embodiments, the preclinical models are selected from MBT-2, Cloudman S91 and EMT6 tumor models.

Identification of an antibody or antigen-binding fragment thereof for therapeutic use may further include carrying out an immune safety assay, which may include, but is not limited to, measuring cytokine release and/or determining the impact of the antibody or antigen-binding fragment on platelet binding, activation, and/or aggregation. In certain embodiments, cytokine release may be measured in vitro using PBMCs or in vivo using a preclinical model such as non-human primates. In certain embodiments, the antibody or antigen-binding fragment thereof does not induce a greater than 10-fold release in IL-6, IFNγ, and/or TNFα levels as compared to levels in an IgG control sample in the immune safety assessment. In certain embodiments, assessment of platelet binding, activation, and aggregation may be carried out in vitro using PBMCs. In some embodiments, the antibody or antigen-binding fragment thereof does not induce a more than 10% increase in platelet binding, activation, and/or aggregation as compared to buffer or isotype control in the immune safety assessment.

The selected antibody or the fragment may be used in the manufacture of a pharmaceutical composition comprising the antibody or the fragment. Such pharmaceutical composition may be used in the treatment of a TGFβ indication in a subject as described herein. For example, the TGFβ indication may be a proliferative disorder, e.g., a TGFβ1-positive cancer. Thus, the invention includes a method for manufacturing a pharmaceutical composition comprising a TGFβ inhibitor, wherein the method includes the step of selecting a TGFβ inhibitor which is tested for immune safety as assessed by immune safety assessment comprising cytokine release assays and optionally further comprising a platelet assay. The TGFβ inhibitor selected by the method does not trigger unacceptable levels of cytokine release (e.g., no more than 10-fold, but more preferably within 2.5-fold as compared to control such as IgG control). Similarly, the TGFβ inhibitor selected by the method does not cause unacceptable levels of platelet aggregation, platelet activation and/or platelet binding. Such TGFβ inhibitor is then manufactured at large-scale, for example 250L or greater, e.g., 1000 L, 2000 L, 3000 L, 4000 L or greater, for commercial production of the pharmaceutical composition comprising the TGFβ inhibitor.

### Cancer/Malignancies

Various cancers involve TGFβ activities, e.g., TGFβ1 activities, and may be treated with the antibodies, compositions, and methods of the present disclosure. As used herein, the term “cancer” comprises any of various malignant neoplasms, optionally associated with TGFβ1-positive cells. Such malignant neoplasms are characterized by the proliferation of anaplastic cells that tend to invade surrounding tissue and metastasize to new body sites and also refers to the pathological condition characterized by such malignant neoplastic growths. The source of TGFβ1 may vary and may include the malignant (cancer) cells themselves, as well as their surrounding or support cells/tissues, including, for example, the extracellular matrix, various immune cells, and any combinations thereof.

Examples of cancer which may be treated in accordance with the present disclosure include but are not limited to, carcinoma, lymphoma, blastoma, sarcoma, and leukemia or lymphoid malignancies. More particular examples of such cancers include, but are not limited to, anal carcinoma; bile duct cancer; brain tumor (including glioblastoma); breast cancer, e.g., HER2+ breast cancer and triple-negative breast cancer (TNBC), ductal carcinoma in situ (DCIS); cervical cancer; colorectal cancer; endometrial or uterine carcinoma; esophageal cancer; gastric or gastrointestinal cancer; gastrointestinal carcinoid tumor; gastrointestinal stromal tumors (GIST); head and neck cancer, e.g. head and neck squamous cell cancer (HNSCC); liver cancer, e.g., hepatocellular carcinoma (HCC); lung cancer, including small-cell lung cancer (SCLC), non-small cell lung cancer (NSCLC), metastatic NSCLC, adenocarcinoma of the lung, and squamous carcinoma of the lung; melanoma; ovarian cancer; pancreatic cancer (e.g., pancreatic ductal adenocarcinoma (PDAC); penile carcinoma; prostate cancer, e.g., castration-resistant prostate cancer (CRPC); renal cell carcinoma (RCC), e.g., clear cell RCC; cancer of the peritoneum; salivary gland carcinoma; thyroid cancer; urothelial carcinoma (UC) of the bladder and urinary tract, including metastatic UC (mUC); urothelial bladder cancer, muscle-invasive bladder cancer (MIBC), and non-muscle-invasive bladder cancer (NMIBC); myeloproliferative neoplasms (MPN), including chronic myeloid leukemia (CML), polycythemia vera (PV), primary myelofibrosis (PMF), essential thrombocythemia (ET), chronic neutrophilic leukemia (CNL); myelodysplastic syndromes (MDS); myeloproliferative neoplasms (MDS/MPN); acute lymphoblastic leukemia (ALL); acute myeloid leukemia (AML); chronic lymphocytic leukemia (CLL); multiple myeloma; Hodgkin's lymphoma; non-Hodgkin's lymphoma (NHL), including diffuse large B cell lymphoma (DLBCL), follicular lymphoma, hairy cell leukemia; mantle cell lymphoma; monoclonal gammopathy of undetermined significance (MGUS); plasma cell myeloma; waldenström macroglobulinemia; and mature T and NK neoplasms. In certain embodiments, a cancer which may be treated in accordance with the present disclosure includes one having high tumor mutational burden.

Affirmative identification of cancer as “TGFβ1-positive” is not required for carrying out the therapeutic methods described herein but is encompassed in some embodiments. Typically, certain cancer types are known to be or suspected, based on credible evidence, to be associated with TGFβ1 signaling.

Cancers may be localized (e.g., solid tumors) or systemic. In the context of the present disclosure, the term “localized” (as in “localized tumor”) refers to anatomically isolated or isolatable abnormalities/lesions, such as solid malignancies, as opposed to systemic disease (e.g., so-called liquid tumors or blood cancers). Certain cancers, such as certain types of leukemia (e.g., myelofibrosis) and multiple myeloma, for example, may have both a localized component (for instance the bone marrow) and a systemic component (for instance circulating blood cells) to the disease. In some embodiments, cancers may be systemic, such as hematological malignancies. Cancers that may be treated according to the present disclosure are TGFβ1-positive and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. In some embodiments, the cancer may be an advanced cancer, such as a locally advanced solid tumor and metastatic cancer.

In some embodiments, the cancer may be a cancer having elevated TGFβ1 levels associated with reactive oxygen species (ROS). In some embodiments, the cancer may be a cancer having elevated ROS levels and expressing high levels of TGFβ1.

Antibodies or antigen-binding fragments thereof encompassed by the present disclosure may be used in the treatment of cancer, including, without limitation: myelofibrosis, melanoma, adjuvant melanoma, renal cell carcinoma (RCC), including clear cell RCC, papillary RCC, chromophobe RCC, collecting duct RCC, or unclassified RCC, bladder cancer, colorectal cancer (CRC) (e.g., microsatellite-stable CRC, mismatch repair deficient colorectal cancer), colon cancer, rectal cancer, anal cancer, breast cancer, triple-negative breast cancer (TNBC), HER2-negative breast cancer, HER2-positive breast cancer, BRCA-mutated breast cancer, hematologic malignancies, non-small cell carcinoma, non-small cell lung cancer/carcinoma (NSCLC), small cell lung cancer/carcinoma (SCLC), extensive-stage small cell lung cancer (ES-SCLC), lymphoma (classical Hodgkin's and non-Hodgkin's), primary mediastinal large B-cell lymphoma (PMBCL), T-cell lymphoma, diffuse large B-cell lymphoma, histiocytic sarcoma, follicular dendritic cell sarcoma, interdigitating dendritic cell sarcoma, myeloma, chronic lymphocytic leukemia (CLL), acute myeloid leukemia (AML), small lymphocytic lymphoma (SLL), head and neck cancer (e.g., head and neck squamous cell cancer), urothelial cancer e.g., metastatic urothelial carcinoma), merkel cell carcinoma (e.g., metastatic merkel cell carcinoma), merkel cell skin cancer, cancer with high microsatellite instability (MSI-H), cancer with mismatch repair deficiency (dMMR), tumor mutation burden high cancer, mesothelioma (e.g., malignant pleural mesothelioma), gastric cancer, gastroesophageal junction cancer (GEJ), gastric adenocarcinoma, neuroendocrine tumors, gastrointestinal stromal tumors (GIST), gastric cardia adenocarcinoma, renal cancer, biliary cancer, cholangiocarcinoma, pancreatic cancer, prostate cancer, adenocarcinoma, squamous cell carcinoma, non-squamous cell carcinoma, cutaneous squamous cell carcinoma (CSCC), ovarian cancer, endometrial cancer, fallopian tube cancer, cervical cancer, peritoneal cancer, stomach cancer, brain cancers, malignant glioma, glioblastoma, gliosarcoma, neuroblastoma, thyroid cancer, adrenocortical carcinoma, oral intra-epithelial neoplasia, esophageal cancer, nasal cavity and paranasal sinus squamous cell carcinoma, nasopharynx carcinoma, salivary gland cancer, liver cancer, basal cell carcinoma; and hepatocellular cancer (HCC). However, any cancer (e.g., patients with such cancer) in which TGFβ1 is overexpressed or is at least a predominant isoform, as determined by, for example biopsy, may be treated with an isoform-selective inhibitor of TGFβ1 in accordance with the present disclosure.

In cancer, TGFβ (e.g., TGFβ1) may be either growth promoting or growth inhibitory. As an example, in pancreatic cancers, SMAD4 wild type tumors may experience inhibited growth in response to TGFβ, but as the disease progresses, constitutively activated type II receptor is typically present. Additionally, there are SMAD4-null pancreatic cancers. In some embodiments, antibodies, antigen binding portions thereof, and/or compositions of the present disclosure are designed to selectively target components of TGFβ signaling pathways that function uniquely in one or more forms of cancer. Leukemias, or cancers of the blood or bone marrow that are characterized by an abnormal proliferation of white blood cells, i.e., leukocytes, can be divided into four major classifications including acute lymphoblastic leukemia (ALL), chronic lymphocytic leukemia (CLL), acute myelogenous leukemia or acute myeloid leukemia (AML) (AML with translocations between chromosome 10 and 11 [t(10, 11)], chromosome 8 and 21 [t(8;21)], chromosome 15 and 17 [t(15;17)], and inversions in chromosome 16 [inv(16)]; AML with multilineage dysplasia, which includes patients who have had a prior myelodysplastic syndrome (MDS) or myeloproliferative disease that transforms into AML; AML and myelodysplastic syndrome (MDS), therapy-related, which category includes patients who have had prior chemotherapy and/or radiation and subsequently develop AML or MDS; d) AML not otherwise categorized, which includes subtypes of AML that do not fall into the above categories; and e) acute leukemias of ambiguous lineage, which occur when the leukemic cells cannot be classified as either myeloid or lymphoid cells, or where both types of cells are present); and chronic myelogenous leukemia (CML).

In some embodiments, any one of the above referenced TGFβ31-positive cancer may also be TGFβ3-positive. In some embodiments, tumors that are both TGFβ31-positive and TGFβ33-positive may be TGFβ1/TGFβ3 co-dominant. In some embodiments, such cancer is carcinoma comprising a solid tumor. In some embodiments, such tumors are breast carcinoma. In some embodiments, the breast carcinoma may be of triple-negative genotype (triple-negative breast cancer). In some embodiments, subjects with TGFβ1-positive cancer have elevated levels of MDSCs. For example, such tumors may comprise MDSCs recruited to the tumor site resulting in an increased number of MDSC infiltrates. In some embodiments, elevated levels of MDSCs may be detected in the blood (i.e., circulating MDSCs). In some embodiments, subjects with breast cancer show elevated levels of C-Reactive Protein (CRP), an inflammatory marker associated with recurrence and poor prognosis. In some embodiments, subjects with breast cancer show elevated levels of IL-6.

The TGFβ inhibitors of the disclosure may be used to treat patients suffering from chronic myeloid leukemia, which is a stem cell disease, in which the BCR/ABL oncoprotein is considered essential for abnormal growth and accumulation of neoplastic cells. Imatinib is an approved therapy to treat this condition; however, a significant fraction of myeloid leukemia patients show Imatinib-resistance. TGFβ inhibition achieved by the inhibitor such as those described herein may potentiate repopulation/expansion to counter BCR/ABL-driven abnormal growth and accumulation of neoplastic cells, thereby providing clinical benefit.

TGFβ inhibitors such as those described herein may be used to treat multiple myeloma. Multiple myeloma is a cancer of B lymphocytes (e.g., plasma cells, plasmablasts, memory B cells) that develops and expands in the bone marrow, causing destructive bone lesions (i.e., osteolytic lesion). Typically, the disease manifests enhanced osteoclastic bone resorption, suppressed osteoblast differentiation (e.g., differentiation arrest) and impaired bone formation, characterized in part, by osteolytic lesions, osteopenia, osteoporosis, hypercalcemia, as well as plasmacytoma, thrombocytopenia, neutropenia and neuropathy. The TGFβ inhibitor therapy described herein may be effective to ameliorate one or more such clinical manifestations or symptoms in patients. The TGFβ1 inhibitor may be administered to patients who receive additional therapy or therapies to treat multiple myeloma, including those listed elsewhere herein. In some embodiments, multiple myeloma may be treated with a TGFβ inhibitor such as an isoform-specific context-independent inhibitor, e.g., Ab6, in combination with a myostatin inhibitor (such as an antibody disclosed in WO 2017/049011, e.g., apitegromab, also known as SRK-015) or an IL-6 inhibitor. In some embodiments, the TGFβ inhibitor may be used in conjunction with traditional multiple myeloma therapies, such as bortezomib, lenalidomide, carfilzomib, pomalidomide, thalidomide, doxorubicin, corticosteroids (e.g., dexamethasone and prednisone), chemotherapy (e.g., melphalan), radiation therapy (including radiotherapeutic agents), stem cell transplantation, plitidepsin, elotuzumab, Ixazomib, masitinib, and/or panobinostat.

The types of carcinomas which may be treated by the methods of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

TGFβ inhibitors such as those described herein may be suited for treating malignancies involving cells of neural crest origin. Cancers of the neural crest lineage (i.e., neural crest-derived tumors) include, but are not limited to: melanoma (cancer of melanocytes), neuroblastoma (cancer of sympathoadrenal precursors), ganglioneuroma (cancer of peripheral nervous system ganglia), medullary thyroid carcinoma (cancer of thyroid C cells), pheochromocytoma (cancer of chromaffin cells of the adrenal medulla), and MPNST (cancer of Schwann cells). In some embodiments, antibodies and methods of the disclosure may be used to treat one or more types of cancer or cancer-related conditions that may include, but are not limited to, colon cancer, renal cancer, breast cancer, malignant melanoma, urothelial carcinoma, and glioblastoma (Schlingensiepen et al., 2008. Cancer Res. 177: 137-50; Ouhtit et al., 2013. J Cancer. 4 (7): 566-572.

### Immunological Characteristics

Under normal conditions, regulatory T cells (Tregs) represent a small subset of the overall CD4-positive lymphocyte population and play key roles for maintaining immune system in homeostasis. In nearly all cancers, however, the number of Tregs is markedly increased. While Tregs play an important role in dampening immune responses in healthy individuals, an elevated number of Tregs in cancer has been associated with poor prognosis. Elevated Tregs in cancer may dampen the host's anti-cancer immunity and may contribute to tumor progression, metastasis, tumor recurrence and/or treatment resistance. For example, human ovarian cancer ascites are infiltrated with Foxp3+ GARP+ Tregs (Downs-Canner et al., Nat Commun. 2017, 8: 14649). Similarly, Tregs positively correlated with a more immunosuppressive and more aggressive phenotype in advanced hepatocellular carcinoma (Kalathil et al., Cancer Res. 2013, 73(8): 2435-44). Tregs can suppress the proliferation of effector T cells. In addition, Tregs exert contact-dependent inhibition of immune cells (e.g., naïve CD4+ T cells) through the production of TGFβ1. To combat a tumor, therefore, it is advantageous to inhibit Tregs so sufficient effector T cells can be available to exert anti-tumor effects.

Increasing lines of evidence suggest the role of macrophages in tumor/cancer progression. The present disclosure encompasses the notion that this is in part mediated by TGFβ activation, especially TGFβ1 activation, in the tumor microenvironment. Bone marrow-derived monocytes (e.g., CD11b+) are recruited to tumor sites in response to tumor-derived cytokines/chemokines (such as CCL2, CCL3 and CCL4), where monocytes undergo differentiation and polarization to acquire pro-cancer phenotype (e.g., M2-biased or M2-like macrophages, TAMs). As previously demonstrated (WO 2018/129329), monocytes isolated from human PBMCs can be induced to polarize into different subtypes of macrophages, e.g., M1 (pro-fibrotic, anti-cancer) and M2 (pro-cancer). A majority of TAMs in many tumors are M2-biased. Among the M2-like macrophages, M2c and M2d subtypes, but not M1, are found to express elevated LRRC33 on the cell surface. Moreover, macrophages can be further skewed or activated by certain cytokine exposure, such as M-CSF, resulting in a marked increase in LRRC33 expression, which coincides with TGFβ1 expression. Increased levels of circulating M-CSF (i.e., serum M-CSF concentrations) in patients with myeloproliferative disease (e.g., myelofibrosis) have also been observed. Generally, tumors with high macrophage (TAM) and/or MDSC infiltrate are associated with poor prognosis. Similarly, elevated levels of M-CSF are also indicative of poor prognosis. Thus, in some embodiments, the TGFβ inhibitors such as those encompassed herein can be used in the treatment of cancer that is characterized by elevated levels of pro-cancer macrophages and/or MDSCs. In some embodiments, the TGFβ inhibitors such as those encompassed herein can be used in the treatment of cancer that is characterized by elevated levels of MDSCs regardless of levels of other macrophages. The LRRC33-arm of the inhibitors may at least in part mediate its inhibitory effects against disease-associated immunosuppressive myeloid cells, e.g., M2-macrophages and MDSCs.

High prevalence of tumor-associated M2-like macrophages is recapitulated in murine syngeneic tumor models, such as those described previously by the Applicant, see data disclosed in PCT/US2019/041373. In MBT-2 tumors, for example, nearly 40% of CD45-positive cells isolated from an established tumor are M2 macrophages. This is reduced by half in animals treated with a combination of an isoform-selective TGFβ1 and anti-PD-1. By comparison, no significant change in the number of tumor-associated M1 macrophages is observed in the same animals. Like M2 macrophages, tumor-associated MDSCs are also elevated in established tumors (about 10-12% of CD45+ cells) and are markedly reduced (to negligible levels) by inhibiting both PD-1 and TGFβ1 in the treated animals. As Applicant previously disclosed, a majority of tumor-infiltrating M2 macrophages and MDSCs express cell-surface LRRC33 and/or LRRC33-proTGFβ1 complex. Interestingly, cell-surface expression of LRRC33 (or LRRC33-proTGFβ1 complex) appears to be highly regulated. The TGFβ inhibitors described herein, e.g., Ab6, are capable of becoming rapidly internalized in cells expressing LRRC33 and proTGFβ1, and the rate of internalization achieved with the TGFβ inhibitor is significantly higher than that with a reference antibody that recognizes cell-surface LRRC33. Similar results are obtained from primary human macrophages. These observations show that Ab6 can promote internalization upon binding to its target, LRRC33-proTGFβ1, thereby removing the LRRC33-containing complexes from the cell surface. Thus, target engagement by a TGFβ inhibitor of the present disclosure, e.g., Ab6 may induce antibody-dependent downregulation of the target protein (e.g., cell-associated proTGFβ1 complexes). At the disease loci, this may reduce the availability of activatable latent LRRC33-proTGFβ1 levels. Therefore, the TGFβ inhibitors of the disclosure may inhibit the LRRC33 arm of TGFβ1 via dual mechanisms of action: i) blocking the release of mature growth factor from the latent complex; and, ii) removing LRRC33-proTGFβ1 complexes from cell-surface via internalization. In the tumor microenvironment, the antibodies may target cell-associated latent proTGFβ1 complexes, augmenting the inhibitory effects on the target cells, such as M2 macrophages (e.g., TAMs), MDSCs, and Tregs. Phenotypically, these are immunosuppressive cells, contributing to the immunosuppressive tumor microenvironment, which is at least in part mediated by the TGFβ1 pathway. Given that many tumors are enriched with these cells, the antibodies that are capable of targeting multiple arms of TGFβ1 function, such as those described herein, should provide a particular functional advantage.

Many human cancers are known to cause elevated levels of MDSCs in patients, as compared to healthy control (reviewed, for example, in Elliott et al., (2017) “Human tumor-infiltrating myeloid cells: phenotypic and functional diversity” Frontiers in Immunology, Vol. 8, Article 86). These human cancers include but are not limited to: bladder cancer, colorectal cancer, prostate cancer, breast cancer, glioblastoma, hepatocellular carcinoma, head and neck squamous cell carcinoma, lung cancer, melanoma, NSCL, ovarian cancer, pancreatic cancer, and renal cell carcinoma. Elevated levels of MDSCs may be detected in biological samples such as peripheral blood mononuclear cell (PBMC) and tissue samples (e.g., tumor biopsy). For example, frequency of or changes in the number of MDSCs may be measured as: percent (%) of total PBMCs, percent (%) of CD14+ cells, percent (%) of CD45+ cells; percent (%) of mononuclear cells, percent (%) of total cells, percent (%) of CD11 b+ cells, percent (%) of monocytes, percent (%) of non-lymphocytic MNCs, percent (%) of KLA-DR cells, using suitable cell surface markers (phenotype).

On the other hand, macrophage infiltration into a tumor may also signify effectiveness of a therapy. As exemplified herein, tumors effectively penetrated by effector T cells (e.g., CD8+ T cells) following the treatment with a combination of a checkpoint inhibitor and a context-independent TGFβ1 inhibitor. Intratumoral effector T cells may lead to recruitment of phagocytic monocytes/macrophages that clean up cell debris.

It was observed that the combination of anti-PD-1 and a TGFβ inhibitor resulted in robust CD8 T cell influx/expansion throughout the tumor, as compared to anti-PD-1 treatment alone. Correspondingly, robust increase in CD8 effector genes may be achieved by the combination treatment. Thus, the TGFβ1 inhibitors of the present disclosure may be used to promote effector T-cell infiltration into tumors.

In addition, extensive infiltration/expansion of the tumor by F4/80-positive macrophages is observed. This may be indicative of M1 (anti-tumor) macrophages clearing cancer cell debris generated by cytotoxic cells and is presumably a direct consequence of TGFβ1 inhibition. As previously described in further detail in PCT/US2019/041373, these tumor-infiltrating macrophages were identified predominantly as non-M2 macrophages for their lack of CD163 expression, indicating that circulating monocytes are recruited to the tumor site upon checkpoint inhibitor and TGFβ1 inhibitor treatment and differentiate into M1 macrophages, and this observation is accompanied by a marked influx of CD8+ T cells into the tumor site. Thus, the TGFβ1 inhibitors of the present disclosure may be used to increase non-M2 macrophages associated with tumor.

Recently, checkpoint blockade therapy (CBT) has become a standard of care for treating a number of cancer types. Despite the profound advances in cancer immunotherapy, primary resistance to CBT remains a major unmet need for patients; a majority of patients' cancers still fail to respond to PD-(L)1 inhibition. Retrospective analysis of urothelial cancer and melanoma tumors has recently implicated TGFβ activation as a potential driver of primary resistance, very likely via multiple mechanisms including exclusion of cytotoxic T cells from the tumor as well as their expansion within the tumor microenvironment (immune exclusion). These observations and subsequent preclinical validation have pointed to TGFβ pathway inhibition as a promising avenue for overcoming primary resistance to CBT. However, therapeutic targeting of the TGFβ pathway has been hindered by dose-limiting preclinical cardiotoxicities, most likely due to inhibition of signaling from one or more TGFβ isoforms.

Many tumors lack of primary response to CBT. In this scenario, CD8+ T cells are commonly excluded from the tumor parenchyma, suggesting that tumors may co-opt the immunomodulatory functions of TGFβ signaling to generate an immunosuppressive microenvironment. These insights from retrospective clinical tumor sample analyses provided the rationale for investigating the role of TGFβ signaling in primary resistance to CBT.

With respect to TGFβ and responses to CBT, herein we observe the prevalent expression of TGFβ1 in many human tumors, suggesting that this family member may be the key driver of this pathway's contribution to primary resistance.

Increasing evidence suggests that TGFβ may be a primary player in creating and/or maintaining immunosuppression in disease tissues, including the immune-excluded tumor environment. Therefore, TGFβ inhibition may unblock the immunosuppression and enable effector T cells (particularly cytotoxic CD8+ T cells) to access and kill target cancer cells. In addition to tumor infiltration, TGFβ inhibition may also promote CD8+ T cell expansion. Such expansion may occur in the lymph nodes and/or in the tumor (intratumorally). While the exact mechanism underlining this process has yet to be elucidated, it is contemplated that immunosuppression is at least in part mediated by immune cell-associated TGFβ1 activation involving regulatory T cells and activated macrophages. It has been reported that TGFβ directly promotes Foxp3 expression in CD4+ T cells, thereby converting them into a regulatory (immunosuppressive) phenotype (i.e., Treg). Moreover, Tregs suppress effector T cell proliferation, thereby reducing immune responses. This process is shown to be TGFβ1-dependent and likely involves GARP-associated TGFβ1 signaling. Observations in both humans and animal models have indicated that an increase in Tregs in TME is associated with poor prognosis in multiple types of cancer. In addition, Applicant has previously shown that M2-polarized macrophages exposed to tumor-derived factors such as M-CSF dramatically upregulate cell-surface expression of LRRC33, which is a presenting molecule for TGFβ1 (see, for example: PCT/US2018/031759). These so-called tumor-associated macrophages (or TAMs) are thought to contribute to the observed TGFβ1-dependent immunosuppression in TMEs and promote tumor growth.

A number of solid tumors are characterized by having tumor stroma enriched with myofibroblasts or myofibroblast-like cells. These cells produce collagenous matrix that surrounds or encases the tumor (such as desmoplasia), which at least in part may be caused by overactive TGFβ1 signaling. It is contemplated that the TGFβ1 activation is mediated via ECM-associated presenting molecules, e.g., LTBP1 and LTBP3 in the tumor stroma.

Selective inhibition of TGFβ activation, such as TGFβ1 inhibition, may be sufficient to overcome primary resistance to CBT. By targeting the prodomain of latent TGFβ1, an isoform-selective inhibitor of TGFβ1 may achieve isoform specificity and inhibit latent TGFβ1 activation.

Selective inhibition of the TGFβ pathway, such as the TGFβ1 pathway, may result in significantly improved preclinical safety versus broad inhibition of all isoform activity. Pleiotropic effects associated with broad TGFβ pathway inhibition have hindered therapeutic targeting of the TGFβ pathway. Most experimental therapeutics to date (e.g., galunisertib, LY3200882, fresolimumab) lack selectivity for a single TGFβ isoform, potentially contributing to the dose-limiting toxicities observed in nonclinical and clinical studies. Genetic data from knockout mice and human loss-of-function mutations in the TGFβ2 or TGFβ3 genes suggest that the cardiac toxicities observed with nonspecific TGFβ inhibitors may be due to inhibition of TGFβ2 or TGFβ3. The present disclosure teaches that selective inhibition of TGFβ1 activation with such an antibody has an improved safety profile and is sufficient to elicit robust antitumor responses when combined with PD-1 blockade, enabling the evaluation of the TGFβ1 inhibitor efficacy at clinically tractable dose levels.

The preclinical studies and results presented herein and in PCT/US2019/041373 demonstrate that combination treatment with a TGFβ1 inhibitor (e.g., Ab6) and a checkpoint inhibitor may have profound effects on the intratumoral immune contexture (e.g., increased levels of tumor-associated CD8+ T cells). These may include an unexpected enrichment of Treg cells by the combination treatment with anti-PD-1/TGFβ1 inhibitor.

In addition to the expected and observed impact on the disposition of cytotoxic T cells within tumors, the TGFβ inhibitor/anti-PD-1 combination treatment may also beneficially impact the immunosuppressive myeloid compartment. Therefore, a therapeutic strategy that includes targeting of these important immunosuppressive cell types may have a greater effect than targeting a single immunosuppressive cell type (i.e., only Treg cells) in the tumor microenvironment. Thus, the TGFβ1 inhibitors of the present disclosure may be used to reduce tumor-associated immunosuppressive cells, such as M2 macrophages and MDSCs.

Applicant's data also demonstrate that highly specific inhibition of TGFβ1 activation may enable the host immune system to overcome a key mechanism of primary resistance to checkpoint blockade therapy, while avoiding the previously recognized toxicities of broader TGFβ inhibition that have been a key limitation for clinical application.

Accordingly, TGFβ inhibitors such as selective TGFβ1 inhibitors may be used to counter primary resistance to CBT, thereby rendering the tumor/cancer more susceptible to the CBT. Such effects may be applicable to treating a wide spectrum of malignancy types, where the cancer/tumor is TGFβ1-positive. In some embodiments, such tumor/cancer may further express additional isoform, such as TGFβ3. Non-limiting examples of the latter may include certain types of carcinoma, such as breast cancer.

Accordingly, the disclosure provides, in some embodiments, selection criteria for identifying or selecting a patient or patient populations/sub-populations for which the TGFβ1 inhibitors are likely to achieve clinical benefit. In some embodiments, suitable phenotypes of human tumors include: i) a subset(s) are shown to be responsive to CBT (e.g., PD-(L)1 axis blockade); ii) evidence of immune exclusion; and/or, iii) evidence of TGFB1 expression and/or TGFβ signaling. Various cancer types fit the profile, including, for example, melanoma and bladder cancer.

As mentioned above, TGFβ inhibitors such as those described herein may be used in the treatment of melanoma. The types of melanoma that may be treated with such inhibitors include, but are not limited to, Lentigo maligna, Lentigo maligna melanoma, Superficial spreading melanoma, Acral lentiginous melanoma, Mucosal melanoma, Nodular melanoma, Polypoid melanoma, and Desmoplastic melanoma. In some embodiments, the melanoma is a metastatic melanoma. In some embodiments, the melanoma is a cutaneous melanoma.

More recently, immune checkpoint inhibitors have been used to effectively treat advanced melanoma patients. In particular, anti-programmed death (PD)-1 antibodies (e.g., nivolumab and pembrolizumab) have now become the standard of care for certain types of cancer such as advanced melanoma, which have demonstrated significant activity and durable response with a manageable toxicity profile. However, effective clinical application of PD-1 antagonists is encumbered by a high rate of innate resistance (˜60-70%) (see Hugo et al., (2016) Cell 165: 35-44), illustrating that ongoing challenges continue to include the questions of patient selection and predictors of response and resistance as well as optimizing combination strategies (Perrot et al., (2013) Ann Dermatol 25(2): 135-144). Moreover, studies have suggested that approximately 25% of melanoma patients who initially responded to an anti-PD-1 therapy eventually developed acquired resistance (Ribas et al., (2016) JAMA 315: 1600-9).

The number of tumor-infiltrating CD8+ T cells expressing PD-1 and/or CTLA-4 appears to be a key indicator of success with checkpoint inhibition, and both PD-1 and CTLA-4 blockade may increase the infiltrating T cells. In patients with higher presence of tumor-associated macrophages, however, anti-cancer effects of the CD8 cells may be suppressed.

It is contemplated that LRRC33-expressing cells, such as myeloid cells, including myeloid precursors, MDSCs and TAMs, may create or support an immunosuppressive environment (such as TME and myelofibrotic bone marrow) by inhibiting T cells (e.g., T cell depletion), such as CD4 and/or CD8 T cells, which may at least in part underline the observed anti-PD-1 resistance in certain patient populations. Indeed, evidence suggests that resistance to anti-PD-1 monotherapy was marked by failure to accumulate CD8+ cytotoxic T cells and reduced Teff/Treg ratio. Notably, the present inventors have recognized that there is a bifurcation among certain cancer patients, such as a melanoma patient population, with respect to LRRC33 expression levels: one group exhibits high LRRC33 expression (LRRC33^{high}), while the other group exhibits relatively low LRRC33 expression (LRRC33^{low}). Thus, the disclosure includes the notion that the LRRC33^{high} patient population may represent those who are poorly responsive to or resistant to immune checkpoint inhibitor therapy. Accordingly, agents that inhibit LRRC33, such as those described herein, may be particularly beneficial for the treatment of cancer, such as melanoma, lymphoma, and myeloproliferative disorders, that is resistant to checkpoint inhibitor therapy (e.g., anti-PD-1).

In some embodiments, a cancer/tumor is intrinsically resistant to or unresponsive to an immune checkpoint inhibitor (e.g., primary resistance). Without intending to be bound by particular theory, the inventors of the present disclosure contemplate that this may be at least partly due to upregulation of TGFβ1 signaling pathways, which may create an immunosuppressive microenvironment where checkpoint inhibitors fail to exert their effects. TGFβ1 inhibition may render such cancer more responsive to checkpoint inhibitor therapy. Non-limiting examples of cancer types which may benefit from a combination of an immune checkpoint inhibitor and a TGFβ1 inhibitor include: myelofibrosis, melanoma, renal cell carcinoma, bladder cancer, colon cancer, hematologic malignancies, non-small cell carcinoma, non-small cell lung cancer/carcinoma (NSCLC), lymphoma (classical Hodgkin's and non-Hodgkin's), head and neck cancer, urothelial cancer e.g., metastatic urothelial carcinoma), cancer with high microsatellite instability, cancer with mismatch repair deficiency, gastric cancer, renal cancer, and hepatocellular cancer. However, any cancer (e.g., patients with such cancer) in which TGFβ1 is overexpressed, is co-expressed with TGFβ3, or is the dominant isoform over TGFβ2/3, as determined by, for example biopsy, may be treated with a TGFβ inhibitor in accordance with the present disclosure.

In some embodiments, a cancer/tumor becomes resistant over time. This phenomenon is referred to as acquired resistance. Like primary resistance, in some embodiments, acquired resistance is at least in part mediated by TGFβ31-dependent pathways. TGFβ inhibitors described herein may be effective in restoring anti-cancer immunity in these cases. The TGFβ inhibitors of the present disclosure may be used to reduce recurrence of tumor. The TGFβ inhibitors of the present disclosure may be used to enhance durability of cancer therapy such as CBT. The term “durability” used in the context of therapies refers to the time between clinical effects (e.g., tumor control) and tumor re-growth (e.g., recurrence). Presumably, durability and recurrence may correlate with secondary or acquired resistance, where the therapy to which the patient initially responded stops working. Thus, the TGFβ inhibitors of the present disclosure may be used to increase the duration of time the cancer therapy remains effective. The TGFβ inhibitors of the present disclosure may be used to reduce the probability of developing acquired resistance among the responders of the therapy. The TGFβ inhibitors of the present disclosure may be used to enhance progression-free survival in patients. In some embodiments, the TGFβ inhibitors described herein may be used to improve disease-free survival time in patients. In some embodiments, the TGFβ inhibitors of the present disclosure may be effective for improving patient-reported outcomes, reduced complications, faster time to treatment completion, more durable treatment, longer time between retreatment, etc. In some embodiments, the TGFβ inhibitors of the present disclosure may be used to improve overall survival in patients.

In some embodiments, the TGFβ inhibitors of the present disclosure may be used to improve rates or ratios of complete verses partial responses among the responders of a cancer therapy. Typically, even in cancer types where response rates to a cancer therapy (such as CBT) are relatively high (e.g., ≥35%), CR rates are quite low. The TGFβ inhibitors of the present disclosure are therefore used to increase the fraction of complete responders within the responder population.

In addition, the TGFβ inhibitor may be also effective to enhance or augment the degree of partial response among partial responders.

In some embodiments, clinical endpoints for the TGFβ inhibitors described herein include those described in the 2018 Food and Drug Administration Guidelines for Clinical Trial Endpoints for the Approval of Cancer Drugs and Biologics, the content of which is incorporated herein in its entirety.

In some embodiments, combination therapy comprising an immune checkpoint inhibitor and an LRRC33 inhibitor (such as those described herein) may be used with the methods disclosed herein and may be effective to treat such cancer. In addition, high LRRC33-positive cell infiltrate in tumors, or otherwise sites/tissues with abnormal cell proliferation, may serve as a biomarker for host immunosuppression and immune checkpoint resistance. Similarly, effector T cells may be precluded from the immunosuppressive niche which limits the body's ability to combat cancer.

As previously disclosed in PCT/US2019/041373, Tregs that express GARP-presented TGFβ1 suppress effector T cell proliferation. Together, TGFβ1 is likely a key driver in the generation and maintenance of an immune inhibitory disease microenvironment (such as TME), and multiple TGFβ1 presentation contexts are relevant for tumors. In some embodiments, the combination therapy may achieve more favorable Teff/Treg ratios.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for treating cancer in a subject in need thereof, said method comprising administering the antibody, or antigen binding portion thereof, to the subject such that the cancer is treated. In certain embodiments, the cancer is colon cancer. In certain embodiments, the cancer is melanoma. In certain embodiments, the cancer is bladder cancer. In certain embodiments, the cancer is head and neck cancer. In certain embodiments, the cancer is lung cancer.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for treating solid tumors. In some embodiments, solid tumors may be desmoplastic tumors, which are typically dense and hard for therapeutic molecules to penetrate. By targeting the ECM component of such tumors, such antibodies may “loosen” the dense tumor tissue to disintegrate, facilitating therapeutic access to exert its anti-cancer effects. Thus, additional therapeutics, such as any known anti-tumor drugs, may be used in combination.

Additionally or alternatively, isoform-specific, context-independent antibodies for fragments thereof that are capable of inhibiting TGFβ1 activation, such as those disclosed herein, may be used in conjunction with the chimeric antigen receptor T-cell (“CAR-T”) technology as cell-based immunotherapy, such as cancer immunotherapy for combatting cancer.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for inhibiting or decreasing solid tumor growth in a subject having a solid tumor, said method comprising administering the antibody, or antigen binding portion thereof, to the subject such that the solid tumor growth is inhibited or decreased. In certain embodiments, the solid tumor is a colon carcinoma tumor. In some embodiments, the antibodies, or antigen binding portions thereof useful for treating a cancer is an isoform-specific, context-independent inhibitor of TGFβ1 activation. In some embodiments, such antibodies target a GARP-TGFβ1 complex, a LTBP1-TGFβ1 complex, a LTBP3-TGFβ1 complex, and a LRRC33-TGFβ1 complex. In some embodiments, such antibodies target a GARP-TGFβ1 complex, a LTBP1-TGFβ1 complex, and a LTBP3-TGFβ1 complex. In some embodiments, such antibodies target a LTBP1-TGFβ1 complex, a LTBP3-TGFβ1 complex, and a LRRC33-TGFβ1 complex. In some embodiments, such antibodies target a GARP-TGFβ1 complex and a LRRC33-TGFβ1 complex.

The disclosure includes the use of TGFβ inhibitors, such as context-independent, isoform-specific inhibitors of TGFβ1, in the treatment of cancer comprising a solid tumor in a subject. In some embodiments, such TGFβ inhibitors may inhibit the activation of TGFβ1. In some embodiments, such TGFβ inhibitors comprise an antibody or antigen-binding portion thereof that binds a proTGFβ1 complex. The binding can occur when the complex is associated with any one of the presenting molecules, e.g., LTBP1, LTBP3, GARP or LRRC33, thereby inhibiting release of mature TGFβ1 growth factor from the complex. In some embodiments, the solid tumor is characterized by having stroma enriched with CD8+ T cells making direct contact with CAFs and collagen fibers. Such a tumor may create an immuno-suppressive environment that prevents anti-tumor immune cells (e.g., effector T cells) from effectively infiltrating the tumor, limiting the body's ability to fight cancer. Instead, such cells may accumulate within or near the tumor stroma. These features may render such tumors poorly responsive to an immune checkpoint inhibitor therapy. As discussed in more detail below, TGFβ1 inhibitors disclosed herein may unblock the suppression so as to allow effector cells to reach and kill cancer cells, for example, used in conjunction with an immune checkpoint inhibitor.

TGFβ, especially TGFβ1, is contemplated to play multifaceted roles in a tumor microenvironment, including tumor growth, host immune suppression, malignant cell proliferation, vascularity, angiogenesis, migration, invasion, metastasis, and chemo-resistance. Each “context” of TGFβ1 presentation in the environment may therefore participate in the regulation (or dysregulation) of disease progression. For example, the GARP axis is particularly important in Treg response that regulates effector T cell response for mediating host immune response to combat cancer cells. The LTBP1/3 axis may regulate the ECM, including the stroma, where cancer-associated fibroblasts (CAFs) play a role in the pathogenesis and progression of cancer. The LRRC33 axis may play a crucial role in recruitment of circulating monocytes to the tumor microenvironment, subsequent differentiation into tumor-associated macrophages (TAMs), infiltration into the tumor tissue and exacerbation of the disease.

In some embodiments, TGFβ1-expressing cells infiltrate the tumor, creating or contributing to an immunosuppressive local environment. The degree by which such infiltration is observed may correlate with worse prognosis. In some embodiments, higher infiltration is indicative of poorer treatment response to another cancer therapy, such as immune checkpoint inhibitors. In some embodiments, TGFβ1-expressing cells in the tumor microenvironment comprise immunosuppressive immune cells such as Tregs and/or myeloid cells. In some embodiments, the myeloid cells include, but are not limited to, macrophages, monocytes (tissue resident or bone marrow-derived), and MDSCs.

In some embodiments, LRRC33-expressing cells in the TME are myeloid-derived suppressor cells (MDSCs). MDSC infiltration (e.g., solid tumor infiltrate) may underline at least one mechanism of immune escape, by creating an immunosuppressive niche from which host's anti-tumor immune cells become excluded. Evidence suggest that MDSCs are mobilized by inflammation-associated signals, such as tumor-associated inflammatory factors, Opon mobilization, MDSCs can influence immunosuppressive effects by impairing disease-combating cells, such as CD8+ T cells and NK cells. In addition, MDSCs may induce differentiation of Tregs by secreting TGFβ and IL-10, further adding to the immunosuppressive effects. Thus, TGFβ inhibitor such as those described herein may be administered to patients with immune evasion (e.g., compromised immune surveillance) to restore or boost the body's ability to fight the disease (such as a cancer or tumor). As described in more detail herein, this may further enhance (e.g., restore or potentiate) the body's responsiveness or sensitivity to another therapy, such as cancer therapy.

In some embodiments, elevated frequencies (e.g., number) of circulating MDSCs in patients are predictive of poor responsiveness to checkpoint blockade therapies, such as PD-1 antagonists and PD-L1 antagonists. For example, biomarker studies showed that circulating pre-treatment HLA-DR^{lo}/CD14+/CD11 b+ myeloid-derived suppressor cells (MDSC) were associated with progression and worse OS (p=0.0001 and 0.0009). In addition, resistance to PD-1 checkpoint blockade in inflamed head and neck carcinoma (HNC) associates with expression of GM-CSF and Myeloid Derived Suppressor Cell (MDSC) markers. This observation suggested that strategies to deplete MDSCs, such as chemotherapy, should be considered in combination (e.g., administered concurrently (e.g., simultaneously), separately, or sequentially) with anti-PD-1. LRRC33 or LRRC33-TGFβ complexes represent a novel target for cancer immunotherapy due to selective expression on immunosuppressive myeloid cells. Therefore, without intending to be bound by particular theory, targeting this complex may enhance the effectiveness of standard-of-care checkpoint inhibitor therapies in the patient population.

The disclosure therefore provides the use of TGFβ inhibitors, such as the isoform-specific TGFβ1 inhibitor described herein, for the treatment of cancer that comprises a solid tumor. Such treatment comprises administration of a TGFβ inhibitor encompassed by the disclosure, e.g., Ab6, to a subject diagnosed with cancer that includes at least one localized tumor (solid tumor) in an amount effective to treat the cancer. Preferably, the subject is further treated with a cancer therapy, such as CBT, chemotherapy, and/or radiation therapy (such as a radiotherapeutic agent). In some embodiments, the TGFβ inhibitor increases the rate/fraction of a primary responder patient population to the cancer therapy. In some embodiments, the TGFβ inhibitor increases the degree of responsiveness of primary responders to the cancer therapy. In some embodiments, the TGF1 inhibitor increases the ratio of complete responders to partial responders to the cancer therapy. In some embodiments, the TGFβ inhibitor increases the durability of the cancer therapy such that the duration before recurrence and/or before the cancer therapy becomes ineffective is prolonged. In some embodiments, the TGFβ inhibitor reduces occurrences or probability of acquired resistance to the cancer therapy among primary responders.

In some embodiments, cancer progression (e.g., tumor proliferation/growth, invasion, angiogenesis and metastasis) may be at least in part driven by tumor-stroma interaction. In particular, CAFs may contribute to this process by secretion of various cytokines and growth factors and ECM remodeling. Factors involved in the process include but are not limited to stromal-cell-derived factor 1 (SCD-1), MMP2, MMP9, MMP3, MMP-13, TNF-α, TGFβ1, VEGF, IL-6, M-CSF. In addition, CAFs may recruit TAMs by secreting factors such as CCL2/MCP-1 and SDF-1/CXCL12 to a tumor site; subsequently, a pro-TAM niche (e.g., hyaluronan-enriched stromal areas) is created where TAMs preferentially attach. Since TGFβ1 has been suggested to promote activation of normal fibroblasts into myofibroblast-like CAFs, administration of an isoform-specific, context-independent TGFβ1 inhibitor such as those described herein may be effective to counter cancer-promoting activities of CAFs. Data previously disclosed in PCT/US2019/041373 suggest that an isoform-specific context-independent antibody that blocks activation of TGFβ1 can inhibit UUO-induced upregulation of maker genes such as CCL2/MCP-1, α-SMA. FN1 and Col1, which are also implicated in many cancers.

In certain embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, are administered to a subject having cancer or a tumor, either alone or in combination with an additional agent, e.g., an anti-PD-1 antibody (e.g., an anti-PD-1 antagonist). Other combination therapies which are included in the disclosure are the administration of an antibody, or antigen binding portion thereof, described herein, with radiation (radiation therapy, including radiotherapeutic agents), or a chemotherapeutic agent (chemotherapy). Exemplary additional agents to use with an anti-TGFβ inhibitor include, but are not limited to, a PD-1 antagonist (e.g., a PD-1 antibody), a PDL1 antagonist (e.g., a PDL1 antibody), a PD-L1 or PDL2 fusion protein, a CTLA4 antagonist (e.g., a CTLA4 antibody), a GITR agonist e.g., a GITR antibody), an anti-ICOS antibody, an anti-ICOSL antibody, an anti-B7H3 antibody, an anti-B7H4 antibody, an anti-TIM3 antibody, an anti-LAG3 antibody, an anti-OX40 antibody (OX40 agonist), an anti-CD27 antibody, an anti-CD70 antibody, an anti-CD47 antibody, an anti-41BB antibody, an anti-PD-1 antibody, an anti-CD20 antibody, an anti-CD3 antibody, an anti-CD3/anti-CD20 bispecific or multispecific antibody, an anti-HER2 antibody, an anti-CD79b antibody, an anti-CD47 antibody, an antibody that binds T cell immunoglobulin and ITIM domain protein (TIGIT), an anti-ST2 antibody, an anti-beta7 integrin (e.g., an anti-alpha4-beta7 integrin and/or alphaE beta7 integrin), a CDK inhibitor, an oncolytic virus, an indoleamine 2,3-dioxygenase (IDO) inhibitor, and/or a PARP inhibitor. Examples of useful oncolytic viruses include, adenovirus, reovirus, measles, herpes simplex, Newcastle disease virus, senecavirus, enterovirus and vaccinia. In certain embodiments, the oncolytic virus is engineered for tumor selectivity.

In some embodiments, determination or selection of therapeutic approach for combination therapy that suits particular cancer types or patient population may involve the following: a) considerations regarding cancer types for which a standard-of-care therapy is available (e.g., immunotherapy-approved indications); b) considerations regarding treatment-resistant subpopulations (e.g., immune excluded); and c) considerations regarding cancers/tumors that are or generally suspected to be “TGFβ1 pathway-active” or otherwise at least in part TGFβ1-dependent (e.g., TGFβ1 inhibition-sensitive). For example, many cancer samples show that TGFβ1 is the predominant isoform by, for instance, TCGA RNAseq. In some embodiments, DNA- and/or RNA-based assays (e.g. RNAseq or Nanostring) may be used to evaluate the level of TGFβ signaling (e.g. TGFβ1 signaling) in tumor samples. In some embodiments, over 50% (e.g., over 50%, 60%, 70%, 80% and 90%) of samples from each tumor type are positive for TGFβ1 isoform expression. In some embodiments, the cancers/tumors that are “TGFβ1 pathway-active” or otherwise at least in part TGFβ1-dependent (e.g., TGFβ1 inhibition-sensitive) contain at least one Ras mutation, such as mutations in K-ras, N-ras and/or H-ras. In some embodiments, the cancer/tumor comprises at least one K-ras mutation.

Confirmation of TGFβ1 expression in clinical samples collected from patients (such as biopsy samples) is not prerequisite to TGFβ1 inhibition therapy, where the particular condition has been generally known or suspected to involve the TGFβ pathway.

In some embodiments, a TGFβ inhibitor such as those described herein is administered in conjunction with checkpoint inhibitory therapy to patients diagnosed with cancer for which one or more checkpoint inhibitor therapies are approved or shown effective. These include, but are not limited to: bladder urothelial carcinoma (such as metastatic urothelial carcinoma), squamous cell carcinoma (such as head & neck), kidney clear cell carcinoma, kidney papillary cell carcinoma, liver hepatocellular carcinoma, lung adenocarcinoma, skin cutaneous melanoma, and stomach adenocarcinoma. In certain embodiments, such patients are poorly responsive or non-responsive to the checkpoint inhibitor therapy. In some embodiments, the poor responsiveness is due to primary resistance. In some embodiments, the cancer that is resistant to checkpoint blockade shows downregulation of TCF7 expression. In some embodiments, TCF7 downregulation in checkpoint inhibition-resistant tumor may be correlated with a low number of intratumoral CD8+ T cells.

A TGFβ inhibitor such as those described herein may be used in the treatment of chemotherapy- or radiotherapy-resistant cancers. Thus, in some embodiments, a TGFβ1 inhibitor, e.g., Ab6, may be administered to patients diagnosed with cancer for which they receive or have received chemotherapy and/or radiation therapy (such as a radiotherapeutic agent). In particular, the use of the TGFβ1 inhibitor is advantageous where the cancer (patient) is resistant to such therapy. In some embodiments, such cancer comprises quiescent tumor propagating cancer cells (TPCs), in which TGFβ signaling controls their reversible entry into a growth arrested state, which protects TPCs from chemotherapy or radiation therapy (such as a radiotherapeutic agent). It is contemplated that upon pharmacological inhibition of TGFβ1, TPCs with compromised fail to enter quiescence and thus rendered susceptible to chemotherapy and/or radiation therapy (such as a radiotherapeutic agent). Such cancer includes various carcinomas, e.g., squamous cell carcinomas. See, for example, Brown et al., (2017) “TGF-β-Induced Quiescence Mediates Chemoresistance of Tumor-Propagating Cells in Squamous Cell Carcinoma.” Cell Stem Cell. 21(5):650-664.

In some embodiments, a TGFβ inhibitor such as an isoform-selective TGFβ1 inhibitor (e.g., Ab6) may be used to treat (e.g., reduce) anemia in a subject, e.g., in a cancer patient. In some embodiments, a TGFβ inhibitor such as an isoform-selective TGFβ1 inhibitor (e.g., Ab6) may be used in combination with a BMP inhibitor (e.g., a BMP6 inhibitor, e.g., a RGMc inhibitor, e.g., any of the RGMc inhibitor disclosed in WO/2020/086736, the content of which is hereby incorporated in its entirety) to treat (e.g., reduce) anemia, e.g., in the subject. In some embodiments, the anemia results from reduced or impaired red blood cell production (e.g., as a result of myelofibrosis or cancer), iron restriction (e.g., as a result of cancer or treatment-induced anemia, such as chemotherapy-induced anemia), or both. In some embodiments, the combination of a TGFβ inhibitor and a BMP inhibitor (antagonist) may be administered at a therapeutically effective amount or amounts that is/are sufficient to relieve one or more anemia-related symptom and/or complication in the subject, e.g., a cancer patient. In some embodiments, the combination of a TGFβ inhibitor and a BMP inhibitor (antagonist) may be administered at a therapeutically effective amount that is sufficient to increase or normalize red blood cell production and/or reduce iron restriction. Without wishing to be bound by theory, it is contemplated that TGFβ1 inhibitors (e.g., Ab6) may alleviate symptoms and/or complications related to anemia through their hematopoiesis-promoting effects and that BMP inhibitors (antagonists) (e.g., a BMP6 inhibitor, e.g., a RGMc inhibitor) may improve iron-deficiency anemia (e.g., chemotherapy-induced anemia). In some embodiments, the treatment for anemia further comprises administering one or more JAK inhibitor (e.g., Jak1/2 inhibitor, Jak1 inhibitor, and/or Jak2 inhibitor).

In some embodiments, the BMP inhibitor is an antagonist of the kinase associated with the BMP receptor (e.g., type I receptor and/or type II receptor).

In some embodiments, the BMP inhibitor is a “ligand trap” that binds (or sequesters) the BMP growth factor(s), including BMP6.

In some embodiments, the BMP inhibitor is an antibody that neutralizes the BMP growth factor(s), including BMP6. Examples include anti-BMP6 antibodies (e.g., WO 2016/098079, Novartis; and, KY-1070, KyMab).

In some embodiments, the BMP inhibitor is an inhibitor of a BMP6 co-receptor, such as RGMc. For example, such inhibitor may include an antibody that binds RGMa/c. (Bdser et al. AAPS J. 2015 July; 17(4): 930-938). More preferably, such inhibitor is an antibody that selectively binds RGMc (see, for example, WO 2020/086736). Therapeutic Indications and/or Subjects Likely to Benefit from a Therapy Comprising a TGFβ-Inhibitor

The current disclosure encompasses methods of treating cancer and predicting or monitoring therapeutic efficacy using a TGFβ inhibitor, e.g., Ab6. In some embodiments, the identification/screening/selection of suitable indications and/or patient populations for which TGFβ inhibitors, such as those described herein, are likely to have advantageous therapeutic benefits comprise: i) whether the disease is driven by or dependent predominantly on the TGFβ1 isoform over the other isoforms in human (or at least co-dominant); ii) whether the condition (or affected tissue) is associated with an immunosuppressive phenotype (e.g., an immune-excluded tumor); and, iii) whether the disease involves both matrix-associated and cell-associated TGFβ1 function.

Differential expression of the three known TGFβ isoforms, namely, TGFβ1, TGFβ2, and TGFβ3, has been observed under normal (healthy; homeostatic) as well as disease conditions in various tissues. Nevertheless, the concept of isoform selectivity has neither been fully exploited nor robustly achieved with conventional approaches that favor pan-inhibition of TGFβ across multiple isoforms. Moreover, expression patterns of the isoforms may be differentially regulated, not only in normal (homeostatic) vs abnormal (pathologic) conditions, but also in different subpopulations of patients. Because most preclinical studies are conducted in a limited number of animal models, which may or may not recapitulate human conditions, data obtained with the use of such models may be biased, resulting in misinterpretations of data or misleading conclusions as to the translatability for purposes of developing therapeutics.

Previous analyses of human tumor samples implicated TGFβ signaling as an important contributor to primary resistance to disease progression and treatment response, including checkpoint blockade therapy (“CBT”) for various types of malignancies. Studies reported in literature reveal that the TGFβ gene expression may be particularly relevant to treatment resistance, suggesting that activity of this isoform may be driving TGFβ signaling in these diseases. As detailed in PCT/US2019/041373, across the majority of human tumor types profiled at The Cancer Genome Atlas (TCGA), TGFB1 expression appears to be the most prevalent, suggesting that selection of preclinical models that more closely recapitulate human disease expression patterns of TGFβ isoforms may be beneficial.

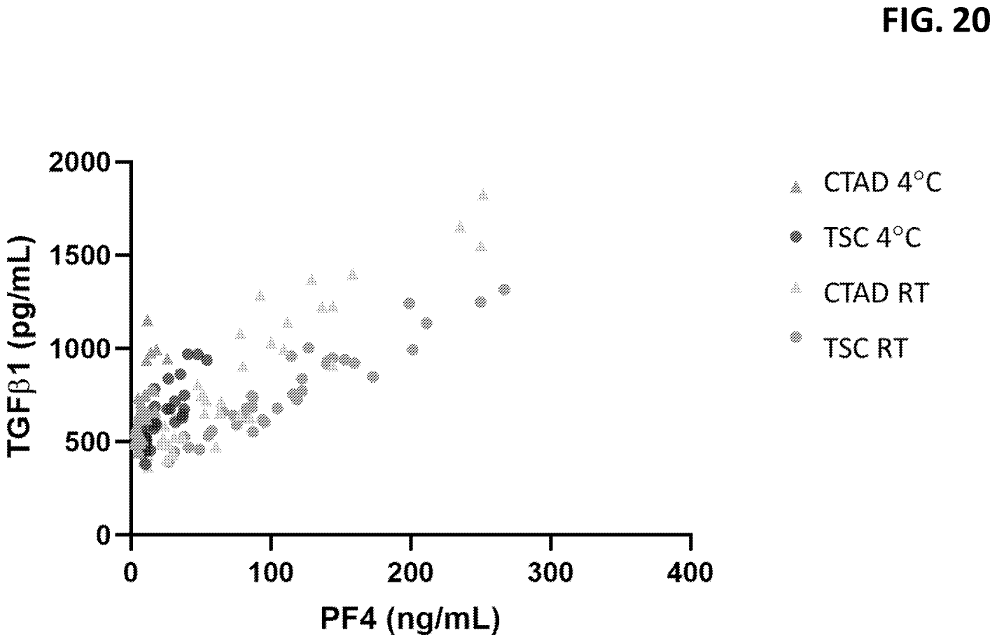

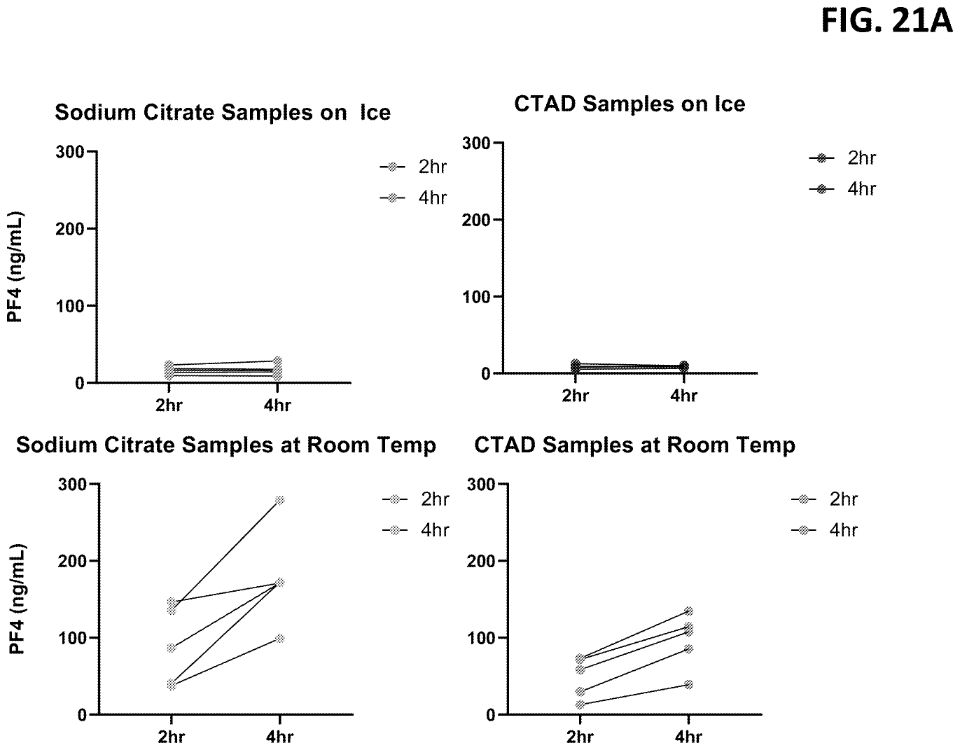

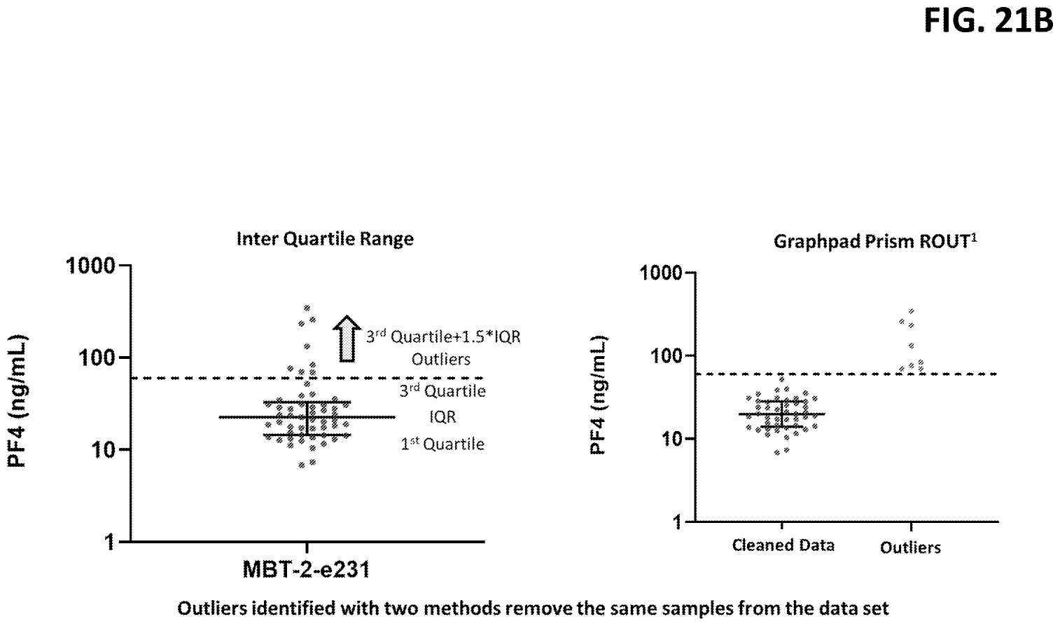

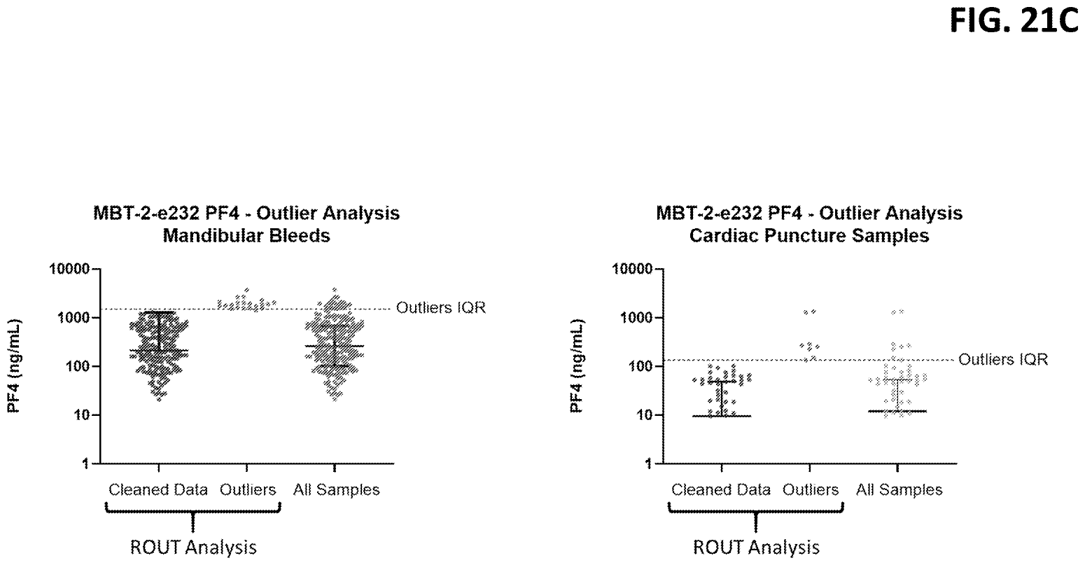

Without being bound by theory, TGFβ1 and TGFβ3 are often co-dominant (co-expressed at similar levels) in certain murine syngeneic cancer models (e.g., EMT-6 and 4T1) that are widely used in preclinical studies (e.g., see PCT/US2019/041373 at FIG. 21B). By contrast, numerous other cancer models (e.g., S91, B16 and MBT-2) express almost exclusively TGFβ1, similar to that observed in many human tumors, in which TGFβ1 appears to be more frequently the dominant isoform over TGFβ2/3 (see PCT/US2019/041373 at FIGS. 20 and 21A). Furthermore, the TGFβ isoform(s) predominantly expressed under homeostatic conditions may not be the disease-associated isoform(s). For example, in normal lung tissues in healthy rats, tonic TGFβ signaling appears to be mediated mainly by TGFβ3. However, TGFβ1 appears to become markedly upregulated in disease conditions, such as lung fibrosis. Taken together, while not prerequisite, it may be beneficial to test or confirm relative expression of TGFβ isoforms in clinical samples so as to select suitable therapeutics to which the patient is likely to respond. In some embodiments, determination of relative isoform expression may be made post-treatment. In such circumstances, patients' responsiveness (e.g., clinical response/benefit) in response to TGFβ1 inhibition therapy may be correlated with relative expression levels of TGFβ isoforms. In some embodiments, overexpression of the TGFβ1 isoform shown ex post facto correlates with greater responsiveness to the treatment.

Whilst inhibition of TGFβ1 alone appears to be sufficient to overcome primary resistance to cancer immunotherapy as demonstrated in a tumor model expressing both TGFβ1 and TGFβ3, findings disclosed herein suggests that inhibition of TGFβ3 may in fact be harmful. Surprisingly, in a murine liver fibrosis model, mice treated with an isoform-selective inhibitor of TGFβ3 manifest exacerbation of fibrosis. A significant increase of collagen deposits in liver sections of these animals suggest that inhibition of TGFβ3 in fact may result in greater dysregulation of the ECM. Without being bound by theory, this suggests that TGFβ3 inhibition may promote a pro-fibrotic phenotype.

A hallmark of pro-fibrotic phenotypes is increased deposition and/or accumulation of collagens in the ECM, which is associated with increased stiffness of tissue ECMs. This has been observed during pathological progression of cancer, fibrosis and cardiovascular disease. Consistent with this, Applicant previously demonstrated the role of matrix stiffness on integrin-dependent activation of TGFβ, using primary fibroblasts grown on silicon-based substrates with defined stiffness (e.g., 5 kPa, 15 kPa or 100 kPa) (see WO 2018/129329). Matrices with greater stiffness enhanced TGFβ1 activation, and this was suppressed by isoform-specific inhibitors of TGFβ1. These observations suggest that the pharmacologic inhibition of TGFβ3 may exert opposing effects to TGFβ1 inhibition by creating a pro-tumor microenvironment, where greater stiffness of the tissue matrix may support cancer progression.

Given the common pathways involved in fibrotic phenotypes and many aspects of cancer progression such as increased invasiveness and metastasis (see, for example: Chakravarthy et al., Nat Com (2018)9:4692. “TGF-β-associated extracellular matrix genes link cancer-associated fibroblasts to immune evasion and immunotherapy failure”), pro-fibrotic effects of TGFβ3 inhibition observed in a fibrosis model may be applicable to cancer contexts.

The finding mentioned above therefore raises the possibility that TGFβ inhibitors with inhibitory potency against TGFβ3 may not only be ineffective in treating cancer but may in fact be detrimental. In some embodiments, TGFβ3 inhibition is avoided in patients suffering from a cancer type that is statistically highly metastatic. Cancer types that are typically considered highly metastatic include, but are not limited to, colorectal cancer, lung cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, and thyroid cancer. Moreover, TGFβ3 inhibition may be best avoided in patients having or are at risk of developing a fibrotic condition and/or cardiovascular disease. Such patients at risk of developing a fibrotic condition and/or cardiovascular disease include, but are not limited to, those with metabolic disorders, such as NAFLD and NASH, obesity, and type 2 diabetes. Similarly, TGFβ3 inhibition may be best avoided in patients diagnosed with or at risk of developing myelofibrosis. Those at risk of developing myelofibrosis include those with one or more genetic mutations implicated in the pathogenesis of myelofibrosis.

In addition to the possible concerns of inhibiting TGFβ3 addressed above, Takahashi et al. (Nat Metab. 2019, 1(2): 291-303) recently reported a beneficial role of TGFβ2 in regulating metabolism. The authors identified TGFβ2 as an exercise-induced adipokine, which stimulated glucose and fatty acid uptake in vitro, as well as tissue glucose uptake in vivo; which improved metabolism in obese mice; and, which reduced high fat diet-induced inflammation. Moreover, the authors observed that lactate, a metabolite released from muscle during exercise, stimulated TGFβ2 expression in human adipocytes and that a lactate-lowering agent reduced circulating TGFβ2 levels and reduced exercise-stimulated improvements in glucose tolerance. Thus, in some embodiments, a TGFβ inhibitor may be used in treating a subject that does not have inhibitory activity towards the TGFβ2 isoform, e.g., to avoid a potentially harmful impact on one or more metabolic functions of a treated subject.

More recently, a potential link between cancer and various metabolic conditions has been recognized. For example, as reviewed by Braun et al., an enhanced risk of cancer mortality is associated with metabolic syndrome among men (Braun et al. Int J Biol Sci. 2011; 7(7): 1003-1015). Similarly, the authors noted “metabolic dysregulation may play an important role in the etiology and progression of certain cancer types and worse outcome for some cancers. Obesity and diabetes, individually, have been associated with breast, endometrial, colorectal, pancreatic, hepatic and renal cancer” (Braun et al. Int J Biol Sci. 2011; 7(7): 1003-1015).

Accordingly, in various embodiments, a TGFβ inhibitor may be used in the treatment of a TGFβ-related indication (e.g., cancer) in a subject, wherein, the TGFβ inhibitor inhibits TGFβ1 but does not inhibit TGFβ2 at the therapeutically effective dose administered. In some embodiments, the subject benefits from improved metabolism after such treatment, wherein optionally, the subject has or is at risk of developing a metabolic disease, such as obesity, high fat diet-induced inflammation, and glucose dysregulation (e.g., diabetes). In some embodiments, the TGFβ-related indication is cancer, wherein optionally the cancer comprises a solid tumor, such as locally advanced cancer and metastatic cancer.

In some embodiments, the TGFβ inhibitor is TGFβ1-selective (e.g., it does not inhibit TGFβ2 and/or TGFβ3 signaling at a therapeutically effective dose). In certain embodiments, a TGFβ1-selective inhibitor is selected for use in treating a cancer patient. In some embodiments, such a treatment: i) avoids TGFβ3 inhibition to reduce the risk of exacerbating ECM dysregulation (which may contribute to tumor growth and invasiveness) and ii) avoids TGFβ2 inhibition to reduce the risk of increasing metabolic burden in the patients. Related methods for selecting a TGFβ inhibitor for therapeutic use are also encompassed herein.

The disclosure includes methods for selecting a TGFβ inhibitor for use in the treatment of cancer, wherein the TGFβ inhibitor has no or little inhibitory potency against TGFβ3 (e.g., the TGFβ inhibitor does not target TGFβ3). In certain embodiments, the TGFβ inhibitor is a TGFβ1-selective inhibitor (e.g., antibodies or antigen binding fragments that do not inhibit TGFβ2 and/or TGFβ3 signaling at therapeutically effective doses). It is contemplated that this selection strategy may reduce the risk of exacerbating ECM dysregulation in cancer patients and still provide benefits of TGFβ1 inhibition to treat cancer. In some embodiments, the cancer patients are also treated with a cancer therapy, such as immune checkpoint inhibitors. In some embodiments, the cancer patient is at risk of developing a metabolic disease, such as fatty liver, obesity, high fat diet-induced inflammation, and glucose or insulin dysregulation (e.g., diabetes).

The present disclosure also includes related methods for selecting and/or treating suitable patient populations who may be candidates for receiving a TGFβ inhibitor capable of inhibiting TGFβ3. Such methods include use of a TGFβ inhibitor capable of inhibiting TGFβ3 for the treatment of cancer in subjects who are not diagnosed with a fibrotic disorder (such as organ fibrosis), who are not diagnosed with myelofibrosis, who are not diagnosed with a cardiovascular disease and/or those who are not at risk of developing such conditions. Similarly, such methods include use of a TGFβ inhibitor capable of inhibiting TGFβ3 for the treatment of cancer in subjects, wherein the cancer is not considered to be highly metastatic. The TGFβ inhibitor capable of inhibiting TGFβ3 may include pan-inhibitors of TGFβ (such as low molecular weight antagonists of TGFβ receptors, e.g., ALK5 inhibitors, and neutralizing antibodies that bind TGFβ1/2/3), isoform-non-selective inhibitors such as antibodies that bind TGFβ1/3 and engineered fusion proteins capable of binding TGFβ1/3, e.g., ligand traps, and integrin inhibitors (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

The surprising notion that TGFβ3 inhibition may in fact be disease-promoting suggests that patients who have been previously treated with or currently undergoing treatment with a TGFβ inhibitor with inhibitory activity towards TGFβ3 may benefit from additional treatment with a TGFβ1-selective inhibitor to counter the possible pro-fibrotic effects of the TGFβ3 inhibitor. Accordingly, the disclosure includes a TGFβ1-selective inhibitor for use in the treatment of cancer in a subject, wherein the subject has been treated with a TGFβ inhibitor that inhibits TGFβ3 in conjunction with a checkpoint inhibitor, comprising the step of: administering to the subject a TGFβ1-selective inhibitor, wherein optionally the cancer is a metastatic cancer, a desmoplastic tumor, myelofibrosis, and/or, wherein the subject has a fibrotic disorder or is at risk of developing a fibrotic disorder and/or cardiovascular disease, wherein optionally the subject at risk of developing a fibrotic disorder or cardiovascular disease suffers from a metabolic condition, wherein optionally the metabolic condition is NAFLD, NASH, obesity or diabetes.

As described herein, the isoform-selective TGFβ1 inhibitors are particularly advantageous for the treatment of diseases in which the TGFβ1 isoform is predominantly expressed relative to the other isoforms (e.g., referred to as TGFβ1-dominant). As an example, a non-limiting list of human cancer clinical samples with relative expression levels of TGFB1 (left), TGFβ2 (center) and TGFβ3 (right) were previously described in PCT/US2019/041373, e.g., at FIGS. 20 and 21A. Each horizontal line across the three isoforms represents a single patient. As can be seen, overall TGFβ1 expression (TGFB1) is significantly higher in most of these human tumors/cancers than the other two isoforms across many tumor/cancer types, suggesting that TGFβ1-selective inhibition may be beneficial in these disease types. Taken together, these lines of evidence support the notion that selective inhibition of TGFβ1 activity may overcome primary resistance to CBT. Generation of highly selective TGFβ1 inhibitors will also enable evaluation of whether such an approach will address key safety issues observed with pan-TGFβ inhibition, which will be important for assessment of their therapeutic utility.

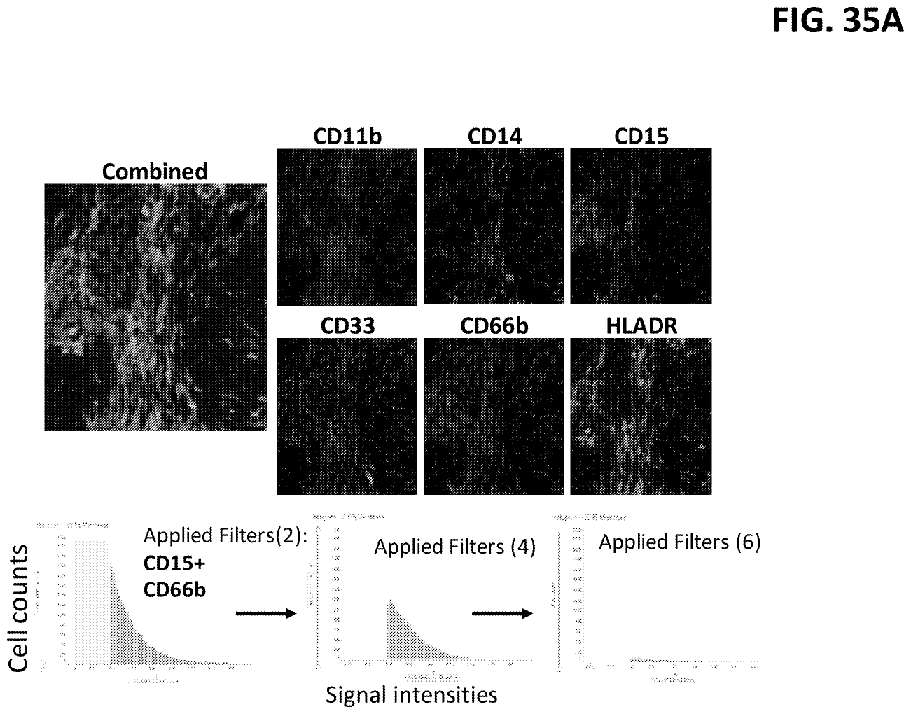

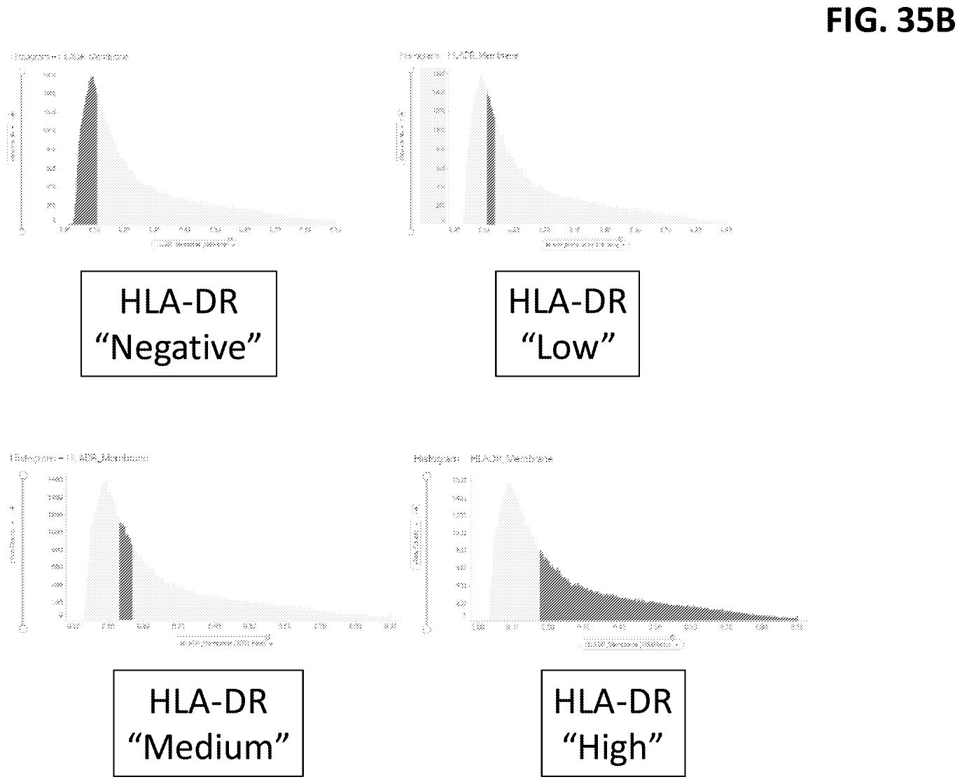

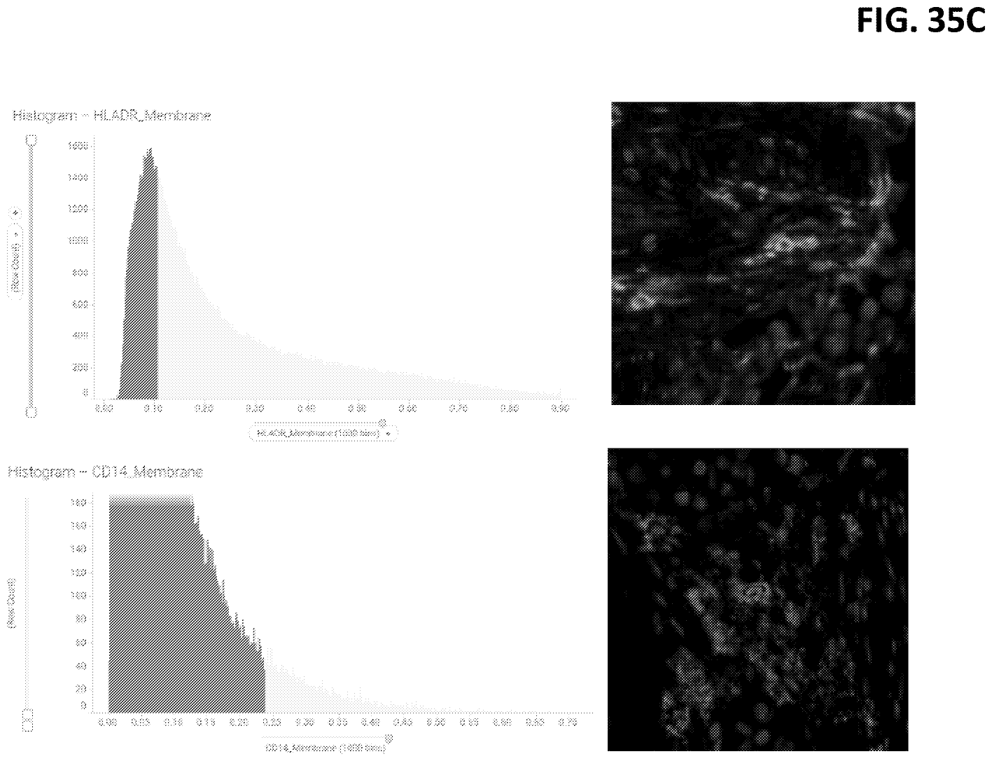

It was previously considered that TGFβ1 inhibitors may not be efficacious, particularly in cancer types in which TGFβ1 is co-dominant with another isoform or in which TGFβ2 and/or TGFβ3 expression is significantly greater than TGFβ1. However more recently, the inventors of the present application have made an unexpected finding that TGFβ inhibitors, e.g., TGFβ1 inhibitors, such as a TGFβ1-selective inhibitor (e.g., Ab6), used in conjunction with a checkpoint inhibitor (e.g., anti-PD-1 antibody), is capable of causing significant tumor regression in the EMT-6 model, which is known to express both TGFβ1 and TGFβ3 at similar levels. The co-dominance has been confirmed by both RNA measurements and ELISA assays (see PCT/US2019/041373 at FIG. 35). This observation was surprising because it had been previously hypothesized that in order to achieve material efficacy in tumors co-expressing TGFβ1 and TGFβ3 in a checkpoint blockade context, both of the co-dominant isoforms would have to be specifically inhibited. Accordingly, methods of treatment disclosed herein include the use of TGFβ1 inhibitor for promoting tumor regression, where the tumor is TGFβ1+/TGFβ3+. Such tumor may include, for example, cancers of epithelial origin, i.e., carcinoma (e.g., basal cell carcinoma, squamous cell carcinoma, renal cell carcinoma, ductal carcinoma in situ (DCIS), invasive ductal carcinoma, and adenocarcinoma). In some embodiments, TGFβ1 is predominantly the disease-associated isoform, whilst TGFβ3 supports homeostatic function in the tissue, such as epithelia.

Aberrant activity of the TGFβ signaling pathway has been reported to impact gene expressions involved in both fibrotic and cancer processes. For instance, dysregulation of the TGFβ1 signal transduction pathway has been observed to alter genes such as SNAI1, MMP2, MMP9, and TIMP1, all of which are important for cellular processes like adhesion and extracellular matrix remodeling and have been implicated in fibrosis and the epithelial mesenchymal transition (EMT) process in cancer. Accordingly, in some embodiments, the methods of treatment herein, e.g., of fibrosis-related cancer indications, comprise the administration of a TGFβ inhibitor that does not inhibit TGFβ3, e.g., using a TGFβ1-selective antibody, e.g., Ab6. Certain tumors, such as various carcinomas, may be characterized as low mutational burden tumors (MBTs). Such tumors are often poorly immunogenic and fail to elicit sufficient T cell response. Cancer therapies that include chemotherapy, radiation therapy (such as a radiotherapeutic agent), cancer vaccines and/or oncolytic virus, may be helpful to elicit T cell immunity in such tumors. Therefore, TGFβ1 inhibition therapy detailed herein can be used in conjunction with one or more of these cancer therapies to increase anti-tumor effects. Essentially, such combination therapy is aimed at converting “cold” tumors (e.g., poorly immunogenic tumors) into “hot” tumors by promoting neo-antigens and facilitating effector cells to attack the tumor. Examples of such tumors include breast cancer, ovarian cancer, and pancreatic cancer, e.g., pancreatic ductal adenocarcinoma (PDAC). Accordingly, any one or more of the antibodies or fragments thereof described herein may be used to treat poorly immunogenic tumor (e.g., an “immune-excluded” tumor) sensitized with a cancer therapy aimed to promote T cell immunity.

In immune-excluded tumors where effector T cells are kept away from the site of tumor (hence “excluded”), the immunosuppressive tumor environment may be mediated in a TGFβ1-dependent fashion. These are tumors that are typically immunogenic; however, T cells cannot sufficiently infiltrate, proliferate, and elicit their cytotoxic effects due to the immune-suppressed environment. Typically, such tumors are poorly responsive to cancer therapies such as CBTs. As suggested by data provided herein and previously in PCT/US2019/041373, adjunct therapy comprising a TGFβ1 inhibitor may overcome the immunosuppressive phenotype, allowing T cell infiltration, proliferation, and anti-tumor function, thereby rendering such tumor more responsive to cancer therapy such as CBT.

Thus, the second inquiry is drawn to identification or selection of patients who have immunosuppressive tumor(s), who are likely to benefit from a TGFβ inhibitor therapy, e.g., a TGFβ1 inhibitor such as Ab6. The presence or the degree of frequencies of effector T cells in a tumor is indicative of anti-tumor immunity. Therefore, detecting anti-tumor cells such as CD8+ cells in a tumor provides useful information for assessing whether the patient may benefit from a CBT and/or TGFβ1 inhibitor therapy.

Detection may be carried out by known methods such as immunohistochemical analysis of tumor biopsy samples, including digital pathology methods. More recently, non-invasive imaging methods are being developed which will allow the detection of cells of interest (e.g., cytotoxic T cells) in vivo. See for example, http://www.imaginab.com/technology/; Tavare et al., (2014) PNAS, 111(3): 1108-1113; Tavare et al., (2015) J Nucl Med 56(8): 1258-1264; Rashidian et al., (2017) J Exp Med 214(8): 2243-2255; Beckford Vera et al., (2018) PLoS ONE 13(3): e0193832; and Tavare et al., (2015) Cancer Res 76(1): 73-82, each of which is incorporated herein by reference. Typically, antibodies or antibody-like molecules engineered with a detection moiety (e.g., radiolabel) can be infused into a patient, which then will distribute and localize to sites of the particular marker (for instance CD8+). In this way, it is possible to determine whether the tumor has an immune-excluded phenotype. If the tumor is determined to have an immune-excluded phenotype, cancer therapy (such as CBT) alone may not be efficacious because the tumor lacks sufficient cytotoxic cells within the tumor environment. Add-on therapy with a TGFβ inhibitor such as those described herein may reduce immuno-suppression thereby rendering the cancer therapy-resistant tumor more responsive to a cancer therapy.

Non-invasive in vivo imaging techniques may be applied in a variety of suitable methods for purposes of diagnosing patients; selecting or identifying patients who are likely to benefit from TGFβ inhibitor therapy, e.g., a TGFβ inhibitor therapy; and/or, monitoring patients for therapeutic response upon treatment. Any cells with a known cell-surface marker may be detected/localized by virtue of employing an antibody or similar molecules that specifically bind to the cell marker. Typically, cells to be detected by the use of such techniques are immune cells, such as cytotoxic T lymphocytes, regulatory T cells, MDSCs, tumor-associated macrophages, NK cells, dendritic cells, and neutrophils. Antibodies or engineered antibody-like molecules that recognize such markers can be coupled to a detection moiety.

Non-limiting examples of suitable immune cell markers include monocyte markers, macrophage markers (e.g., M1 and/or M2 macrophage markers), CTL markers, suppressive immune cell markers, MDSC markers (e.g., markers for G- and/or M-MDSCs), including but are not limited to: CD8, CD3, CD4, CD11 b, CD33, CD163, CD206, CD68, CD14, CD15, CD66b, CD34, CD25, and CD47. In some embodiments, the in vivo imaging comprises T cell tracking, such as cytotoxic CD8-positive T cells. Accordingly, any one of the TGFβ inhibitors of the present disclosure may be used in the treatment of cancer in a subject with a solid tumor, wherein the treatment comprises: i) carrying out an in vivo imaging analysis to detect T cells in the subject, wherein optionally the T cells are CD8+ T cells, and if the solid tumor is determined to be an immune-excluded solid tumor based on the in vivo imaging analysis of step (i), then, administering to the subject a therapeutically effective amount of a TGFβ inhibitor, e.g., Ab6. In some embodiments, the subject has received a CBT, wherein optionally the solid tumor is resistant to the CBT. In some embodiments, the subject is administered with a CBT in conjunction with the TGFβ1 inhibitor, as a combination therapy. The combination may comprise administration of a single formulation that comprises both a checkpoint inhibitor and a TGFβ inhibitor. The TGFβ inhibitor may be a TGFβ1 inhibitor, such as a TGFβ1-selective inhibitor, e.g., Ab6, an isoform-non-selective inhibitor, e.g., low molecular weight ALK5 antagonists, neutralizing antibodies that bind two or more of TGFβ1/2/3, e.g., GC1008 and variants, antibodies that bind TGFβ1/3, ligand traps, e.g., TGFβ1/3 inhibitors, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). Alternatively, the combination therapy may comprise administration of a first formulation comprising a checkpoint inhibitor and a second formulation comprising a TGFβ inhibitor, wherein the TGFβ inhibitor may be a TGFβ1 inhibitor, such as a TGFβ31-selective inhibitor, e.g., Ab6, an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, a ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

In some embodiments, the in vivo imaging comprises MDSC tracking, such as G-MDSCs and M-MDSCs. For example, MDSCs may be enriched at a disease site (such as fibrotic tissues and solid tumors) at the baseline. Upon therapy (e.g., TGFβ1 inhibitor therapy), fewer MDSCs may be observed, as measured by reduced intensity of the label (such as radioisotope and fluorescence), indicative of therapeutic effects.

In some embodiments, the in vivo imaging comprises tracking or localization of LRRC33-positive cells. LRRC33-positive cells include, for example, MDSCs and activated M2-like macrophages (e.g., TAMs and activated macrophages associated with fibrotic tissues). For example, LRRC33-positive cells may be enriched at a disease site (such as fibrotic tissues and solid tumors) at the baseline. Upon therapy (e.g., TGFβ1 inhibitor therapy), fewer cells expressing cell surface LRRC33 may be observed, as measured by reduced intensity of the label (such as radioisotope and fluorescence), indicative of therapeutic effects.

In some embodiments, the in vivo imaging comprises the use of PET-SPECT, MRI and/or optical fluorescence/bioluminescence in order to detect target of interest (e.g., molecules or entities which can be bound by the labeled reagent, such as cells and tissues expressing appropriate marker(s)).

In some embodiments, labeling of antibodies or antibody-like molecules with a detection moiety may comprise direct labeling or indirect labeling.

In some embodiments, the detection moiety may be a tracer. In some embodiments, the tracer may be a radioisotope, wherein optionally the radioisotope may be a positron-emitting isotope. In some embodiments, the radioisotope is selected from the group consisting of: ¹⁸F. ¹¹C ¹³N, ¹⁵O, ⁶⁸Ga, ¹⁷⁷Lu, ¹⁸F and ⁸⁹Zr.

Thus, such methods may be employed to carry out in vivo imaging with the use of labeled antibodies in immune-PET.

In some embodiments, such in vivo imaging is performed for monitoring a therapeutic response to the TGFβ1 inhibition therapy in the subject. For example, the therapeutic response may comprise conversion of an immune excluded tumor into an inflamed tumor, which correlates with increased immune cell infiltration into a tumor. This may be visualized by increased intratumoral immune cell frequency or degree of detection signals, such as radiolabeling and fluorescence.

Accordingly, the disclosure includes a method for treating cancer which may comprise the following steps: i) selecting a patient diagnosed with cancer comprising a solid tumor, wherein the solid tumor is or is suspected to be an immune excluded tumor; and, ii) administering to the patient an antibody or the fragment encompassed herein in an amount effective to treat the cancer. In some embodiments, the patient has received, or is a candidate for receiving a cancer therapy such as immune checkpoint inhibition therapies (e.g., PD-(L)1 antibodies), chemotherapies, radiation therapies, engineered immune cell therapies, and cancer vaccine therapies. In some embodiments, the selection step (i) comprises detection of immune cells or one or more markers thereof, wherein optionally the detection comprises a tumor biopsy analysis, serum marker analysis, and/or in vivo imaging.

In some embodiments, the patient is diagnosed with cancer for which a CBT has been approved, wherein optionally, statistically a similar patient population with the particular cancer shows relatively low response rates to the approved CBT, e.g., under 25%. For example, the response rates for the CBT may be between about 10-25%, for example about 10-15%. Such cancer may include, for example, ovarian cancer, gastric cancer, and triple-negative breast cancer. The TGFβ inhibitors of the present disclosure may be used in the treatment of such cancer, where the subject has not yet received a CBT. The TGFβ1 inhibitor may be administered to the subject in combination with a CBT. In some embodiments, the subject may receive or may have received additional cancer therapy, such as chemotherapy and radiation therapy (including a radiotherapeutic agent).

In vivo imaging techniques described above may be employed to detect, localize, and/or track certain MDSCs in a patient diagnosed with a TGFβ-associated disease, such as cancer. Healthy individuals have no or low frequency of MDSCs in circulation. With the onset of or progression of such a disease, elevated levels of circulating and/or disease-localized MDSCs may be detected. For example, CCR2-positive M-MDSCs have been reported to accumulate to tissues with inflammation and may cause progression of fibrosis in the tissue (such as pulmonary fibrosis), and this is shown to correlate with TGFβ1 expression. Similarly, MDSCs are enriched in a number of solid tumors (including triple-negative breast cancer) and in part contribute to the immunosuppressive phenotype of the TME. Therefore, treatment response to TGFβ inhibition, such as TGFβ1 inhibition, according to the present disclosure may be monitored by localizing or tracking circulating MDSCs. Reduction of or low frequency of circulating MDSC levels is typically indicative of therapeutic benefits or better prognosis. Accordingly, the current disclosure provides methods of predicting and monitoring therapeutic efficacy of TGFβ inhibitor therapy, e.g., combination therapy of a TGFβ1 inhibitor and a checkpoint inhibitor, by measuring circulating MDSCs in the blood or a blood component of the subject. The current disclosure also provides methods of selecting patients, e.g., patients with immunosuppressive cancers and determining treatment regimens based on levels of circulating MDSCs measured. The TGFβ inhibitor may be a TGFβ1 inhibitor, such as a TGFβ1-selective inhibitor, e.g., Ab6, an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, a ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

The TGFβ inhibitors of the present disclosure may be used in the treatment of cancer in a subject, wherein the cancer is characterized by immune suppression, wherein the cancer optionally comprises a solid tumor that is TGFβ1-positive and TGFβ3-positive. Such subject may be diagnosed with carcinoma. In some embodiments, the carcinoma is breast carcinoma, wherein optionally the breast carcinoma is triple-negative breast cancer (TNBC). Such treatment can further comprise a cancer therapy, including, without limitation, chemotherapies, radiation therapies, cancer vaccines, engineered immune cell therapies (such as CAR-T), and immune checkpoint blockade therapies, such as anti-PD(L)-1 antibodies. The TGFβ inhibitor may be a TGFβ1 inhibitor, such as a TGFβ1-selective inhibitor, e.g., Ab6, or an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, a ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3).

In some embodiments, a cold tumor is identified, in which few effector cells are present both inside and outside the tumor or is known to be a type of cancer characterized as poorly immunogenic (e.g., a tumor characterized as an immune desert). A subject/patient with such a tumor is treated with an immune-sensitizing cancer therapy, such as chemotherapy, radiation therapy (such as a radiotherapeutic agent), oncolytic viral therapy, and cancer vaccine, in order to elicit stronger T cell response to tumor antigens (e.g., neo-antigens). This step may convert the cold tumor into an “immune excluded” tumor. The subject optionally further receives a CBT, such as anti-PD-(L)1. The subject is further treated with a TGFβ1 inhibitor, such as the antibodies disclosed herein. This may convert the cold or immune excluded tumor into an “inflamed” or “hot” tumor, which confers responsiveness to immunotherapy. Non-limiting examples of poorly immunogenic cancers include breast cancer (such as TNBC), prostate cancer (such as Castration resistant prostate cancer (CRPC)) and pancreatic cancer (such as pancreatic adenocarcinoma (PDAC)).

As Applicant has previously shown, high affinity, isoform-selective inhibitors of TGFβ1 of the present disclosure, such as Ab6, can inhibit Plasmin-induced activation of TGFβ1. The plasmin-plasminogen axis has been implicated in certain tumorigenesis, invasion and/or metastasis, of various cancer types, carcinoma in particular, such as breast cancer. Therefore, it is possible that the TGFβ inhibitors such as those described herein may exert the inhibitory effects via this mechanism in tumors or tumor models, such as EMT6, involving the epithelia. Indeed, Plasmin-dependent destruction or remodeling of epithelia may contribute to the pathogenesis of conditions involving epithelial injuries and invasion/dissemination of carcinoma. The latter may be triggered by epithelial to mesenchymal transition (“EMT”). It has been reported that plasminogen activation and plasminogen-dependent invasion were more prominent in epithelial-like cells and were partly dictated by the expression of S100A10 and PAI-1 (Bydoun et al., (2018) Scientific Reports, 8:14091).

The TGFβ inhibitors of the present disclosure (e.g., a TGFβ1 inhibitor, e.g., Ab6) may be used in the treatment of anemia in a subject in need thereof. In some embodiments, the subject is diagnosed with cancer. In some embodiments, the subject is diagnosed with a myeloproliferative disorder (e.g., myelofibrosis). In some embodiments, a TGFβ inhibitor (e.g., Ab6) is used alone to treat anemia. In some embodiments, the TGFβ inhibitor is used in combination with an additional agent, e.g., a BMP antagonist (e.g., a BMP6 inhibitor, e.g., a RGMc inhibitor). In some embodiments, a combination comprising a TGFβ1 inhibitor (e.g., Ab6) and a BMP antagonist (e.g., a BMP6 inhibitor, e.g., a RGMc inhibitor) is used to improve anemia resulting from insufficient erythrocyte production, iron deficiency, and/or chemotherapy. In some embodiments, the treatment for anemia further comprises administering one or more JAK inhibitor (e.g., Jak1/2 inhibitor, Jak1 inhibitor, and/or Jak2 inhibitor).

The disclosure includes a method for selecting a patient population or a subject who is likely to respond to a therapy comprising a TGFβ inhibitor such as those described herein. Subjects selected according to such methods may be the subjects treated according to the various aspects of the present disclosure. Such method may comprise the steps of: providing a biological sample (e.g., clinical sample) collected from a subject, determining (e.g., measuring or assaying) relative levels of TGFβ1, TGFβ2 and TGFβ3 in the sample, and, administering to the subject a composition comprising a TGFβ inhibitor, such as a TGFβ1 inhibitor described herein, if TGFβ1 is the dominant isoform over TGFβ2 and TGFβ3; and/or, if TGFβ1 is significantly overexpressed or upregulated as compared to control. In some embodiments, such method comprises the steps of obtaining information on the relative expression levels of TGFβ1, TGFβ2 and TGFβ3 which was previously determined; identifying a subject to have TGFβ1-positive, preferably TGFβ1-dominant, disease; and administering to the subject a composition comprising a TGFβ inhibitor disclosed herein. In some embodiments, such subject has a disease (such as cancer) that is resistant to a therapy (such as cancer therapy). In some embodiments, such subject shows intolerance to the therapy and therefore has or is likely to discontinue the therapy. Addition of the TGFβ inhibitor to the therapeutic regimen may enable reducing the dosage of the first therapy and still achieve clinical benefits in combination. In some embodiments, the TGFβ inhibitor may delay or reduce the need for surgeries. In some embodiments, the TGFβ inhibitor is a TGFβ1 inhibitor described herein, e.g., Ab6.

Relative levels of the isoforms may be determined by RNA-based assays and/or protein-based assays, which are well-known in the art. In some embodiments, the step of administration may also include another therapy, such as immune checkpoint inhibitors, or other agents provided elsewhere herein. Such methods may optionally include a step of evaluating a therapeutic response by monitoring changes in relative levels of TGFβ1, TGFβ2 and TGFβ3 at two or more time points. In some embodiments, clinical samples (such as biopsies) are collected both prior to and following administration. In some embodiments, clinical samples (such as biopsies) are collected multiple times following treatment to assess in vivo effects over time.

In addition to the above inquiries, the third inquiry interrogates the breadth of TGFβ function, such as TGFβ1 function, involved in a particular disease. In particular, this may be represented by the number of TGFβ1 contexts, namely, which presenting molecule(s) mediate disease-associated TGFβ1 function. TGFβ1-specific, broad-context inhibitors, such as context-independent inhibitors, are advantageous for the treatment of diseases that involve both an ECM component and an immune component of TGFβ1 function. Such disease may be associated with dysregulation in the ECM as well as perturbation in immune cell function or immune response. Thus, the TGFβ1 inhibitors described herein are capable of targeting ECM-associated TGFβ1 (e.g., presented by LTBP1 or LTBP3) as well as immune cell-associated TGFβ1 (e.g., presented by GARP or LRRC33). Such inhibitors inhibit all four of the therapeutic targets (e.g., “context-independent” inhibitors): GARP-associated pro/latent TGFβ1; LRRC33-associated pro/latent TGFβ1; LTBP1-associated pro/latent TGFβ1; and, LTBP3-associated pro/latent TGFβ1, so as to broadly inhibit TGFβ1 function in these contexts.

Whether or not a particular condition of a patient involves or is driven by multiple aspects of TGFβ1 function may be assessed by evaluating expression profiles of the presenting molecules, in a clinical sample collected from the patient. Various assays are known in the art, including RNA-based assays and protein-based assays, which may be performed to obtain expression profiles. Relative expression levels (and/or changes/alterations thereof) of LTBP1, LTBP3, GARP, and LRRC33 in the sample(s) may indicate the source and/or context of TGFβ1 activities associated with the condition. For instance, a biopsy sample taken from a solid tumor may exhibit high expression of all four presenting molecules. For example, LTBP1 and LTBP3 may be highly expressed in CAFs within the tumor stroma, while GARP and LRRC33 may be highly expressed by tumor-associated immune cells, such as Tregs and leukocyte infiltrate, respectively.

Accordingly, the disclosure includes a method for determining (e.g., testing or confirming) the involvement of TGFβ1 in the disease, relative to TGFβ2 and TGFβ3. In some embodiments, the method further comprises a step of: identifying a source (or context) of disease-associated TGFβ1. In some embodiments, the source/context is assessed by determining the expression of TGFβ presenting molecules, e.g., LTBP1, LTBP3, GARP and LRRC33 in a clinical sample taken from patients. In some embodiments, such methods are performed ex post facto.

With respect to LRRC33-positive cells, Applicant of the present disclosure has recognized that there can be a significant discrepancy between RNA expression and protein expression of LRRC33. In particular, while a select cell type appears to express LRRC33 at the RNA level, only a subset of such cells express the LRRC33 protein on the cell-surface. It is contemplated that LRRC33 expression may be highly regulated via protein trafficking/localization, for example, in terms of plasma membrane insertion and rapid internalization. Therefore, in certain embodiments, LRRC33 protein expression may be used as a marker associated with a diseased tissue (such as tumor tissues) enriched with, for example, activated/M2-like macrophages and MDSCs.

In a related aspect, the present disclosure provides therapeutic use and related treatment methods comprising an immune checkpoint inhibitor, e.g., a PD-(L)1 antibody. Non-limiting examples of useful checkpoint inhibitors include: ipilimumab (Yervoy®); nivolumab (Opdivo®); pembrolizumab (Keytruda®); avelumab (Bavencio®); cemiplimab (Libtayo®); atezolizumab (Tecentriq®); budigalimab (ABBV-181); durvalumab (Imfinzi®), etc.

According to the present disclosure, a cancer treatment method may include a checkpoint inhibitor for use in the treatment of cancer in a subject, wherein the treatment comprises administration of a checkpoint inhibitor to the subject who is treated with a TGFβ inhibitor or vice versa (i.e., administration of a TGFβ inhibitor to a subject who is treated with a checkpoint inhibitor), wherein, upon treatment of the TGFβ inhibitor, circulating MDSC levels in a sample collected from the subject are reduced, as compared to prior to the treatment. The sample may be a blood sample or a sample of blood component. The checkpoint inhibitor may be a PD-1 antibody. The checkpoint inhibitor may be a PD-L1 antibody. The checkpoint inhibitor may be a CTLA4 antibody. In some embodiments, the checkpoint inhibitor is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181); and durvalumab (e.g., Imfinzi®).

According to the present disclosure, a cancer treatment method may include a checkpoint inhibitor for use in the treatment of cancer in a subject who is poorly responsive to the checkpoint inhibitor, or wherein the subject has a cancer with primary resistant to the checkpoint inhibitor, wherein the treatment comprises administering to the subject a TGFβ inhibitor, measuring circulating MDSC levels before and after the administration of the TGFβ inhibitor, and if circulating MDSCs are reduced after the TGFβ inhibitor administration, further administering a checkpoint inhibitor to the subject in an amount sufficient to treat cancer. The checkpoint inhibitor may be a PD-1 antibody. The checkpoint inhibitor may be a PD-L1 antibody. The checkpoint inhibitor may be a CTLA4 antibody. In some embodiments, the checkpoint inhibitor is selected from the group consisting of ipilimumab (e.g., Yervoy®); nivolumab (e.g., Opdivo®); pembrolizumab (e.g., Keytruda®); avelumab (e.g., Bavencio®); cemiplimab (e.g., Libtayo®); atezolizumab (e.g., Tecentriq®); budigalimab (ABBV-181); and durvalumab (e.g., Imfinzi®). Optionally, the TGFβ inhibitor is an isoform-selective inhibitor of TGFβ1, wherein optionally the inhibitor is an activation inhibitor of TGFβ1 or neutralizing antibody that selectively binds TGFβ1; or an isoform-non-selective inhibitor (e.g., inhibitors of TGFβ1/2/3, TGFβ1/3, TGFβ1/2).

### Combination Therapy

Disclosed herein are pharmaceutical compositions of a TGFβ inhibitor, e.g., an antibody or antigen-binding portion thereof, described herein, and related methods used as, or referring to, combination therapies for treating subjects who may benefit from TGFβ inhibition in vivo. In any of these embodiments, such subjects may receive combination therapies that include a first composition comprising at least one TGFβ inhibitor, e.g., Ab6, in conjunction with at least a second composition comprising at least one additional therapeutic intended to treat the same or overlapping disease or clinical condition. In some embodiments, such subjects may receive an additional third composition comprising at least one additional therapeutic intended to treat the same or overlapping disease or clinical condition. The TGFβ inhibitor may be a TGFβ1 inhibitor, such as a TGFβ1-selective inhibitor (e.g., one which does not inhibit TGFβ2 and/or TGFβ3 signaling at a therapeutically effective dose), e.g., Ab6, or an isoform-non-selective inhibitor, e.g., a low molecular weight ALK5 antagonist, a neutralizing antibody that bind two or more of TGFβ1/2/3, e.g., GC1008 or variants, an antibody that bind TGFβ1/3, ligand trap, e.g., a TGFβ1/3 inhibitor, and/or an integrin inhibitor (e.g., an antibody that binds to αVβ1, αVβ3, αVβ5, αVβ6, αVβ8, α5β1, αIIbβ3, or α8β1 integrins, and inhibits downstream activation of TGFβ. e.g., selective inhibition of TGFβ1 and/or TGFβ3). The first, second, and third compositions may both act on the same cellular target, or discrete cellular targets. In some embodiments, the first, second, and third compositions may treat or alleviate the same or overlapping set of symptoms or aspects of a disease or clinical condition. In some embodiments, the first, second, and third compositions may treat or alleviate a separate set of symptoms or aspects of a disease or clinical condition. In some embodiments, the combination therapy may comprise more than three compositions, which may act on the same target or discrete cellular targets, and which may treat or alleviate the same or overlapping set of symptoms or aspects of a disease or clinical condition. To give but one example, the first composition may treat a disease or condition associated with TGFβ signaling, while the second composition may treat inflammation or fibrosis associated with the same disease, etc. As another example, the first composition may treat a disease or condition associated with TGFβ signaling, while the second and third compositions may have anti-neoplastic effects and/or help reverse immune suppression. In certain embodiments, the first composition may be a TGFβ inhibitor (e.g., a TGFβ1 inhibitor described herein), the second composition may be a checkpoint inhibitor, and the third composition may be a checkpoint inhibitor distinct from the second composition. In certain embodiments, a first composition comprising a TGFβ inhibitor (e.g., a TGFβ1 inhibitor described herein) is combined with a checkpoint inhibitor and a chemotherapeutic agent. In certain embodiments, a first composition comprising a TGFβ inhibitor (e.g., a TGFβ1 inhibitor described herein) is combined with two distinct checkpoint inhibitors and a chemotherapeutic agent. Such combination therapies may be administered in conjunction with each other. As noted above, the phrase “in conjunction with,” in the context of combination therapies, means that therapeutic effects of a first therapy overlap temporally and/or spatially with therapeutic effects of a second therapy in the subject receiving the combination therapy. The first, second, and/or additional compositions may be administered concurrently (e.g., simultaneously), separately, or sequentially. Thus, the combination therapies may be formulated as a single formulation for concurrent or simultaneous administration, or as separate formulations for concurrent (e.g., simultaneous), separate, or sequential administration of the therapies. As used herein, a combination therapy may comprise two or more therapies (e.g., compositions) given in a single bolus or administration, or in a single patient visit (e.g., to or with a medical professional) but in two or more separate boluses or administrations, or in separate patient visits (and, e.g., in two or more separate boluses or administrations). For instance, the therapies may be given less than about 5 minutes apart, or 1 minute apart. The therapies may be given less than about 30 minutes or 1 hour apart (e.g., in a single patient visit). In some embodiments, the therapies may be given more than about 1 minute, about 2 minutes, about 5 minutes, about 10 minutes, about 15 minutes, about 30 minutes, about 45 minutes, about 1 hour, about 2 hour, about 4 hours, about 6 hours, about 8 hours, about 10 hours, about 1 day, about 2 day, about 3 days, about 5 days, about 1 week, about 2 weeks, about 3 weeks, about 1 month, or more, apart. In some embodiments, the therapies may be given more than about 1 day apart (e.g., in separate visits). The therapies may be given within 3 months (e.g., within 1 month) of one another. In some embodiments, a therapy may be given according to the dosing schedule of one or more approved therapeutics fortreating the condition (e.g., administered at the same frequency as for an approved checkpoint inhibitor or other chemotherapeutic agent).

In certain embodiments, the TGFβ inhibitor (e.g., a TGFβ1 inhibitor described herein) may be administered in an amount of about 3000 mg, 2400 mg, 2000 mg, 1600 mg, 800 mg, 240 mg, 80 mg, or less. In certain embodiments, the TGFβ inhibitor (e.g., Ab6) may be administered in an amount of about 3000 mg, 2400 mg, 2000 mg, 1600 mg, 800 mg, 240 mg, 80 mg, or less, e.g., at a frequency of once every two weeks, three weeks, or any multiples of two weeks or three weeks (e.g., once every four weeks, once every six weeks), wherein the TGFβ inhibitor (e.g., Ab6) is administered alone or in combination with a checkpoint inhibitor therapy, (e.g., any approved checkpoint inhibitor therapy, including, but not limited to, antibodies or other agents against cytotoxic T-lymphocyte antigen-4 (CTLA-4), programmed cell death protein 1 (PD-1), programmed cell death receptor ligand 1 (PD-L1), T-cell immunoglobulin domain and mucin domain-3 (TIM3), lymphocyte-activation gene 3 (LAG3), killer cell immunoglobulin-like receptor (KIR), glucocorticoid-induced tumor necrosis factor receptor (GITR), or V-domain immunoglobulin (Ig)-containing suppressor of T-cell activation (VISTA)).

In certain embodiments, the TGFβ inhibitor (e.g., Ab6) may be administered alone at 3000 mg once every six weeks. In certain embodiments, the TGFβ inhibitor (e.g., Ab6) may be administered at 3000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every six weeks. In certain embodiments, the TGFβ inhibitor (e.g., Ab6) may be administered at 2400 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every six weeks. In certain embodiments, the TGFβ inhibitor (e.g., Ab6) may be administered at 2000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every six weeks. In certain embodiments, the TGFβ inhibitor (e.g., Ab6) may be administered at 1600 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every six weeks. In certain embodiments, the TGFβ inhibitor (e.g., Ab6) may be administered at 800 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every six weeks. In certain embodiments, the TGFβ inhibitor (e.g., Ab6) may be administered at 240 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every six weeks. In certain embodiments, the TGFβ inhibitor (e.g., Ab6) may be administered at 80 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every six weeks. In certain embodiments, the TGFβ inhibitor (e.g., Ab6) may be administered at an amount of less than 80 mg once every six weeks, wherein the TGFβ inhibitor (e.g., Ab6) is administered in combination with an anti-PD-(L)1 therapy. In some embodiments, the TGFβ inhibitor (e.g., Ab6) may be administered every six weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every four weeks. In certain embodiments, the TGFβ inhibitor (e.g., Ab6) may be administered at 3000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every four weeks. In certain embodiments, the TGFβ inhibitor (e.g., Ab6) may be administered at 2400 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every four weeks. In certain embodiments, the TGFβ inhibitor (e.g., Ab6) may be administered at 2000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every four weeks. In certain embodiments, the TGFβ inhibitor (e.g., Ab6) may be administered at 1600 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every four weeks. In certain embodiments, the TGFβ inhibitor (e.g., Ab6) may be administered at 800 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every four weeks. In certain embodiments, the TGFβ inhibitor (e.g., Ab6) may be administered at 240 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every four weeks. In certain embodiments, the TGFβ inhibitor (e.g., Ab6) may be administered at 80 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every four weeks. In certain embodiments, the TGFβ inhibitor (e.g., Ab6) may be administered at an amount of less than 80 mg once every four weeks, wherein the TGFβ inhibitor (e.g., Ab6) is administered in combination with an anti-PD-(L)1 therapy. In some embodiments, the TGFβ inhibitor (e.g., Ab6) may be administered every four weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every three weeks. In certain embodiments, the TGFβ inhibitor (e.g., Ab6) may be administered at 3000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every three weeks. In certain embodiments, the TGFβ inhibitor (e.g., Ab6) may be administered at 2400 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every three weeks. In certain embodiments, the TGFβ inhibitor (e.g., Ab6) may be administered at 2000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every three weeks. In certain embodiments, the TGFβ inhibitor (e.g., Ab6) may be administered at 1600 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every three weeks. In certain embodiments, the TGFβ inhibitor (e.g., Ab6) may be administered at 800 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 800 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every three weeks. In certain embodiments, the TGFβ inhibitor (e.g., Ab6) may be administered at 240 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every three weeks. In certain embodiments, the TGFβ inhibitor (e.g., Ab6) may be administered at 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every three weeks. In certain embodiments, the TGFβ inhibitor (e.g., Ab6) may be administered at an amount of less than 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of three weeks, wherein the TGFβ inhibitor (e.g., Ab6) is administered in combination with an anti-PD-(L)1 therapy. In some embodiments, the TGFβ inhibitor (e.g., Ab6) may be administered every three weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every two weeks. In certain embodiments, the TGFβ inhibitor (e.g., Ab6) may be administered at 3000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every two weeks. In certain embodiments, the TGFβ inhibitor (e.g., Ab6) may be administered at 2400 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every two weeks. In certain embodiments, the TGFβ inhibitor (e.g., Ab6) may be administered at 2000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every two weeks. In certain embodiments, the TGFβ inhibitor (e.g., Ab6) may be administered at 1600 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every two weeks. In certain embodiments, the TGFβ inhibitor (e.g., Ab6) may be administered at 800 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 800 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every two weeks. In certain embodiments, the TGFβ inhibitor (e.g., Ab6) may be administered at 240 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every two weeks. In certain embodiments, the TGFβ inhibitor (e.g., Ab6) may be administered at 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every two weeks. In certain embodiments, the TGFβ inhibitor (e.g., Ab6) may be administered at an amount of less than 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of two weeks, wherein the TGFβ inhibitor (e.g., Ab6) is administered in combination with an anti-PD-(L)1 therapy. In some embodiments, the TGFβ inhibitor (e.g., Ab6) may be administered every two weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, a TGFβ inhibitor (e.g., a TGFβ1 inhibitor described herein, e.g, Ab6) may be administered in combination or in conjunction with a genotoxic therapy. Without wishing to be bound by theory, in some embodiments, a subject receiving a genotoxic therapy may have elevated levels of TGFβ1, for example where administration of the genotoxic therapy may cause an increase in TGFβ1 levels in the subject. Thus, in some embodiments, administration of a TGFβ inhibitor (e.g., a TGFβ1 inhibitor described herein, e.g, Ab6) in combination or in conjunction with a genotoxic therapy may provide added therapeutic benefit by binding to this increased TGFβ1 following treatment with the genotoxic therapy. In some embodiments, the genotoxic therapy may be a chemotherapy and/or a radiation therapy.

In certain embodiments, a TGFβ inhibitor (e.g., a TGFβ1 inhibitor described herein, e.g, Ab6) may be administered in combination or in conjunction with checkpoint inhibitor therapy, e.g., an anti-PD-(L)1 therapy), to a subject who is a non-responder to checkpoint inhibitor therapy, e.g., an anti-PD-(L)1 therapy). In certain embodiments, the subject has not previously received a checkpoint inhibitor therapy. Exemplary checkpoint inhibitors include, but are not limited to, nivolumab (Opdivo®, anti-PD-1 antibody), pembrolizumab (Keytruda®, anti-PD-1 antibody), cemiplimab (Libtayo®, anti-PD-1 antibody), budigalimab (ABBV-181, anti-PD-1 antibody); BMS-936559 (anti-PD-L1 antibody), atezolizumab (Tecentriq®, anti-PD-L1 antibody), avelumab (Bavencio®, anti-PD-L1 antibody), durvalumab (Imfinzi®, anti-PD-L1 antibody), ipilimumab (Yervoy®, anti-CTLA4 antibody), tremelimumab (anti-CTLA4 antibody), IMP-321 (eftilgimod alpha or “ImmuFact®”, anti-LAG3 large molecule), BMS-986016 (Relatlimab, anti-LAG3 antibody), and lirilumab (anti-KIR2DL-1, -2, -3 antibody).

In certain embodiments, the TGFβ inhibitor (e.g., Ab6) is administered alone to a subject having advanced solid cancer. In certain embodiments, the TGFβ inhibitor (e.g., Ab6) is administered in combination with a checkpoint inhibitor therapy to a subject having advanced solid cancer. In certain embodiments, the TGFβ inhibitor (e.g., Ab6) is administered in combination with a checkpoint inhibitor therapy to a subject having advanced solid cancer, wherein the subject is a non-responder to prior checkpoint inhibitor therapy. In some embodiments, the subject has non-small cell lung cancer (NSCLC), melanoma (MEL), or urothelial carcinoma (UC), including metastatic urothelial carcinoma (mUC). In some embodiments, the subject has ovarian cancer, colorectal cancer (CRC), bladder cancer, renal cell carcinoma (RCC), including clear cell RCC, papillary RCC, chromophobe RCC, collecting duct RCC, or unclassified RCC, or head and neck cancer (e.g., head and neck squamous cell carcinoma (HNSCC) or oropharynx cancer)). In some embodiments, the subject has esophageal cancer, gastric cancer, hepatocellular carcinoma (HCC), triple-negative breast cancer (TNBC), cervical cancer, endometrial cancer, basal cell carcinoma (BCC), cutaneous squamous cell carcinoma (CSCC), merkel cell carcinoma (MCC), small-cell lung cancer (SCLC), primary mediastinal large B-cell lymphoma (PMBCL), Hodgkin's lymphoma, microsatellite instability high cancer (MSI-H) (e.g., MSI-H CRC), mismatch repair deficient cancer (dMMR)(e.g., dMMR CRC), tumor mutational burden-high (TMB-H) cancer, or malignant pleural mesothelioma (MPM). In certain embodiments, the TGFβ inhibitor (e.g., Ab6) is administered in combination with a checkpoint inhibitor therapy to a subject having urothelial carcinoma (UC), including metastatic urothelial carcinoma (mUC), melanoma (MEL), or non-small cell lung cancer NSCLC. In certain embodiments, the subject is a non-responder to checkpoint inhibitor therapy. In certain embodiments, the checkpoint inhibitor therapy is pembrolizumab (e.g., Keytruda®). In certain embodiments, the checkpoint inhibitor therapy is nivolumab (e.g., Opdivo®). In certain embodiments, the checkpoint inhibitor therapy is cemiplimab (e.g., Libtayo®). In certain embodiments, the checkpoint inhibitor therapy is atezolizumab (e.g., Tecentriq®). In certain embodiments, the checkpoint inhibitor therapy is avelumab (e.g., Bavencio®). In certain embodiments, the checkpoint inhibitor therapy is durvalumab (e.g., Imfinzi®). In certain embodiments, the checkpoint inhibitor therapy is budigalimab (e.g., ABBV-181).

In certain embodiments, the TGFβ inhibitor (e.g., Ab6) is administered at 3000 mg, 2400 mg, 2000 mg, 1600 mg, 800 mg, 240 mg, 80 mg, or less in combination with pembrolizumab at a frequency of once every two weeks, three weeks, or any multiples of two weeks or three weeks (e.g., once every four weeks, once every six weeks). In certain embodiments, the TGFβ inhibitor (e.g., Ab6) is administered in combination with pembrolizumab to a subject having NSCLC, UC, MEL, esophageal cancer, gastric cancer, HNSCC, HCC, cervical cancer, SCLC, PMBCL, Hodgkin's lymphoma, MSI-H or dMMR cancer, or TMB-H cancer. In certain embodiments, the subject is a non-responder to pembrolizumab. In certain embodiments, the subject has not received pembrolizumab previously. In certain embodiments, the TGFβ inhibitor (e.g., Ab6) is administered in combination with pembrolizumab to a subject having NSCLC who is a non-responder to pembrolizumab treatment. In certain embodiments, the subject having NSCLC has not received pembrolizumab previously. In certain embodiments, the TGFβ inhibitor (e.g., Ab6) is administered in combination with pembrolizumab to a subject having MEL who is a non-responder to pembrolizumab treatment. In certain embodiments, the subject having MEL has not received pembrolizumab previously. In certain embodiments, the TGFβ inhibitor (e.g., Ab6) is administered in combination with pembrolizumab to a subject having UC or mUC who is a non-responderto pembrolizumab treatment. In certain embodiments, the subject having UC or mUC has not received pembrolizumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with nivolumab at a frequency of once every two weeks, three weeks, or any multiples of two weeks or three weeks (e.g., once every four weeks, once every six weeks). In certain embodiments, the TGFβ inhibitor (e.g., Ab6) is administered in combination with nivolumab to a subject having NSCLC, UC, MEL, esophageal cancer, HNSCC, HCC, RCC, Hodgkin's lymphoma, MSI-H or dMMR CRC, or MPM. In certain embodiments, the subject is a non-responder to nivolumab. In certain embodiments, the subject has not received nivolumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with cemiplimab at a frequency of once every two weeks, three weeks, or any multiples of two weeks or three weeks (e.g., once every four weeks, once every six weeks). In certain embodiments, the TGFβ inhibitor (e.g., Ab6) is administered in combination with cemiplimab to a subject having BCC or CSCC. In certain embodiments, the subject is a non-responder to cemiplimab. In certain embodiments, the subject has not received cemiplimab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with atezolizumab at a frequency of once every two weeks, three weeks, or any multiples of two weeks or three weeks (e.g., once every four weeks, once every six weeks). In certain embodiments, the TGFβ inhibitor (e.g., Ab6) is administered in combination with atezolizumab to a subject having NSCLC, MEL, HCC, TNBC, or SCLC. In certain embodiments, the subject is a non-responder to atezolizumab. In certain embodiments, the subject has not received atezolizumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with avelumab at a frequency of once every two weeks, three weeks, or any multiples of two weeks or three weeks (e.g., once every four weeks, once every six weeks). In embodiments, the TGFβ inhibitor (e.g., Ab6) is administered in combination with avelumab to a subject having UC or MCC. In certain embodiments, the subject is a non-responder to avelumab. In certain embodiments, the subject has not received avelumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with durvalumab at a frequency of once every two weeks, three weeks, or any multiples of two weeks or three weeks (e.g., once every four weeks, once every six weeks). In embodiments, the TGFβ inhibitor (e.g., Ab6) is administered in combination with durvalumab to a subject having NSCLC or SCLC. In certain embodiments, the subject is a non-responder to durvalumab. In certain embodiments, the subject has not received durvalumab previously.

In certain embodiments, the TGFβ inhibitor (e.g., Ab6) is administered at 3000 mg, 2400 mg, 2000 mg, 1600 mg, 800 mg, 240 mg, 80 mg, or less in combination with budigalimab (e.g., ABBV-181), e.g., at a dose of 250 mg, 375 mg, or 500 mg at a frequency of once every two weeks, three weeks, or any multiples of two weeks or three weeks (e.g., once every four weeks, once every six weeks). In certain embodiments, budigalimab (e.g., ABBV-181) is administered at 250 mg once every two weeks. In certain embodiments, budigalimab (e.g., ABBV-181) is administered at 375 mg once every three weeks. In certain embodiments, budigalimab (e.g., ABBV-181) is administered at 500 mg once every four weeks. In certain embodiments, the TGFβ inhibitor (e.g., Ab6) is administered in combination with budigalimab to a subject having a locally advanced or metastatic solid tumor. See, e.g., NCT 03821935 (Study to determine the safety, tolerability, pharmacokinetics and recommended phase 2 dose (RP2D) of ABBV-151 as a single agent and in combination with ABBV-181 in participants with locally advanced or metastatic solid tumors. https://clinicaltrials.gov/ct2/show/NCT03821935). In certain embodiments, budigalimab is administered once every four weeks. In certain embodiments, the TGFβ inhibitor (e.g., Ab6) is administered in combination with budigalimab to a subject having triple-negative breast cancer (TNBC), pancreatic adenocarcinoma, urothelial cancer, or Hepatocellular carcinoma (HCC). In certain embodiments, the TGFβ inhibitor (e.g., Ab6) is administered in combination with budigalimab to a subject having non-small cell lung cancer or head and neck squamous cell carcinoma. Italiano et al. Cancer Immunol Immunother. 2022 February; 71(2):417-431. In certain embodiments, the subject is a non-responder to budigalimab. In certain embodiments, the subject has not received budigalimab previously.

In certain embodiments, combination therapies produce synergistic effects in the treatment of a disease. The term “synergistic” refers to effects that are greater than additive effects (e.g., greater efficacy) of each monotherapy in aggregate.

In some embodiments, combination therapies comprising a pharmaceutical composition described herein produce efficacy that is overall equivalent to that produced by another therapy (such as monotherapy of a second agent) but are associated with fewer unwanted adverse effect or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients.

The disclosure provides pharmaceutical compositions and methods for use in, and as, combination therapies for the reduction of TGFβ1 protein activation and the treatment or prevention of diseases or conditions associated with TGFβ1 signaling, as described herein. Accordingly, the methods or the pharmaceutical compositions may further comprise a second therapy. In some embodiments, the methods or pharmaceutical compositions disclosed herein may further comprise a third therapy. In some embodiments, the second therapy and/or the third therapy may be useful in treating or preventing diseases or conditions associated with TGFβ1 signaling. The second therapy and/or the third therapy may diminish or treat at least one symptom(s) associated with the targeted disease. The first, second, and third therapies may exert their biological effects by similar or unrelated mechanisms of action; or either one or both of the first and second therapies may exert their biological effects by a multiplicity of mechanisms of action. In some embodiments, the second therapy and a TGFβ inhibitor disclosed herein (e.g., a TGFβ1-selective inhibitor disclosed herein) are present in a single formulation or in separate formulations contained within in a single package or kit. In some embodiments, the second therapy, the third therapy, and a TGFβ inhibitor disclosed herein (e.g., a TGFβ1-selective inhibitor disclosed herein) are present in a single formulation or in separate formulations contained within in a single package or kit. In some embodiments, the second therapy, and a TGFβ inhibitor disclosed herein (e.g., a TGFβ1-selective inhibitor disclosed herein) are comprised in a single molecule, e.g., in a bispecific antibody or other multispecific construct or, wherein the checkpoint inhibitor is a small molecule, in an antibody-drug conjugate. In some embodiments, the second therapy, the third therapy, and a TGFβ inhibitor disclosed herein (e.g., a TGFβ1-selective inhibitor disclosed herein) are comprised in a single molecule, e.g., in a bispecific antibody or other multispecific construct or, wherein the checkpoint inhibitor is a small molecule, in an antibody-drug conjugate. Examples of engineered constructs with TGFβ inhibitory activities include M7824 (Bintrafusp alfa) and AVID200. M7824 is a bifunctional fusion protein composed of 2 extracellular domains of TGF-βRII (a TGF-β “trap”) fused to a human IgG1 monoclonal antibody against PD-L1. AVID200 is an engineered TGF-β ligand trap comprised of TGF-β receptor ectodomains fused to a human Fc domain.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered concurrently (e.g., simultaneously), separately, or sequentially within described embodiments.

The one or more anti-TGFβ antibodies, or antigen binding portions thereof, of the disclosure may be used in conjunction with one or more of additional therapeutic agents. Examples of the additional therapeutic agents which can be used with an anti-TGFβ antibody of the disclosure include, but are not limited to: cancer vaccines, engineered immune cell therapies, chemotherapies, radiation therapies (e.g., radiotherapeutic agents), a modulator of a member of the TGFβ superfamily, such as a myostatin inhibitor (e.g., a myostatin inhibitor disclosed in WO2016/073853 and WO2017/049011, the contents of which are hereby incorporated in their entirety), and a GDF11 inhibitor; a VEGF agonist; a VEGF inhibitor (such as bevacizumab); an IGF1 agonist; an FXR agonist; a CCR2 inhibitor; a CCR5 inhibitor; a dual CCR2/CCR5 inhibitor; CCR4 inhibitor, a lysyl oxidase-like-2 inhibitor; an ASK1 inhibitor; an Acetyl-CoA Carboxylase (ACC) inhibitor; a p38 kinase inhibitor; pirfenidone; nintedanib; an M-CSF inhibitor (e.g., M-CSF receptor antagonist and M-CSF neutralizing agents); a MAPK inhibitor (e.g., Erk inhibitor), an immune checkpoint agonist or antagonist; an IL-11 antagonist; and IL-6 antagonist, and the like. Other examples of the additional therapeutic agents which can be used with the TGFβ inhibitors include, but are not limited to, an indoleamine 2,3-dioxygenase (IDO) inhibitor, an arginase inhibitor, a tyrosine kinase inhibitor, Ser/Thr kinase inhibitor, a dual-specific kinase inhibitor. In some embodiments, such an agent may be a PI3K inhibitor, a PKC inhibitor, or a JAK inhibitor.

While checkpoint inhibitor (CPI) therapies have transformed the treatment of solid tumors, less than half of cancer patients are eligible for treatment with an approved CPI and of those, <13% respond to CPI therapy (Haslam 2019). Given these data, there remains a significant unmet need across solid tumor indications with approved and unapproved therapies.

Recent data suggest that the effectiveness of immunomodulatory strategies require the presence of a baseline immune response. Tumors lacking a pre-existing immune response or tumors with low numbers of T cells in the tumor core and an enrichment of T cells in the invasive margin or stroma (e.g., in an immune-excluded tumor) have been associated with poor response to CPI (Galon and Bruni 2019. Nat Rev Drug Discov. 18(3): 197-218). The TGFβ pathway has been implicated in mediating primary resistance to CPI therapies, and as such, combination therapy with an anti-latent TGFβ monoclonal antibody may increase efficacy in patients with an inadequate response to CPI monotherapy.

The current disclosure includes use of a TGFβ inhibitor, e.g., Ab6, as a potential anti-cancer therapy alone or in combination with other therapies for the treatment of solid tumors and rare hematological malignancies for which TGFβ signaling dysregulation has been implicated as a mediator of the disease process. In some embodiments, combination therapy comprising a TGFβ inhibitor, e.g., Ab6, and at least one additional agent may be efficacious in patients with advanced solid tumors such as cutaneous melanoma, urothelial carcinoma (UC), non-small cell lung cancer (NSCLC), and head and neck cancer. In some embodiments, combination therapy comprising a TGFβ inhibitor, e.g., Ab6, and at least one additional agent may be efficacious in patients with immune-excluded tumors such as non-small cell lung cancer, melanoma, renal cell carcinoma, triple-negative breast cancer, gastric cancer, microsatellite stable-colorectal cancer, pancreatic cancer, small cell lung cancer, HER2-positive breast cancer, or prostate cancer.

In some embodiments, the at least one additional agent (e.g., cancer therapy agent) used in a method or composition disclosed herein is a checkpoint inhibitor. In some embodiments, the at least one additional agent is selected from the group consisting of a PD-1 antagonist, a PD-L1 antagonist, a PD-L1 or PD-L2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody (OX40 agonist), an anti-CD27 antibody, an anti-CD70 antibody, an anti-CD47 antibody, an anti-41BB antibody, an anti-PD-1 antibody, an oncolytic virus, and a PARP inhibitor. Exemplary checkpoint inhibitors include, but are not limited to, nivolumab (Opdivo®, anti-PD-1 antibody), pembrolizumab (Keytruda®, anti-PD-1 antibody), cemiplimab (Libtayo®, anti-PD-1 antibody), budigalimab (ABBV-181, anti-PD-1 antibody), BMS-936559 (anti-PD-L1 antibody), atezolizumab (Tecentriq®, anti-PD-L1 antibody), avelumab (Bavencio®, anti-PD-L1 antibody), durvalumab (Imfinzi®, anti-PD-L1 antibody), ipilimumab (Yervoy®, anti-CTLA4 antibody), tremelimumab (anti-CTLA4 antibody), IMP-321 (eftilgimod alpha or “ImmuFact®”, anti-LAG3 large molecule), BMS-986016 (Relatlimab, anti-LAG3 antibody), and lirilumab (anti-KIR2DL-1, -2, -3 antibody). In some embodiments, the TGFβ inhibitors disclosed herein is used in the treatment of cancer in a subject who is a poor responder or non-responder of a checkpoint inhibition therapy, such as those listed herein. In some embodiments, the checkpoint inhibitor and a TGFβ inhibitor (e.g., a TGFβ1-selective inhibitor disclosed herein) are comprised in a single molecule, e.g., in a bispecific antibody or other multispecific construct or, wherein the checkpoint inhibitor is a small molecule, in an antibody-drug conjugate.

In some embodiments, the disclosure encompasses use of a TGFβ inhibitor, e.g., Ab6, in combination with at least one checkpoint inhibitor therapy for the treatment of solid tumors and/or hematological malignancies for which TGFβ signaling dysregulation has been implicated as a mediator of the disease process. In certain embodiments, the combination therapy may be administered to patients who are not responsive to checkpoint inhibitor therapy (e.g., anti-PD-1 or anti-PD-L1 therapy). Such patients may include, but are not limited to, those diagnosed with non-small cell lung cancer, urothelial bladder carcinoma, melanoma, triple-negative breast cancer, or other advance solid cancers. In certain embodiments, the combination therapy may comprise a TGFβ inhibitor, e.g., Ab6, and a checkpoint inhibitor therapy (e.g., pembrolizumab). In certain embodiments, the combination therapy may be administered to immunotherapy-naïve patients (e.g., patients who have not previously received a checkpoint inhibitor therapy) diagnosed with a cancer that has received FDA approval for treatment with a checkpoint inhibitor therapy. Such cancer may be gastric cancer (e.g., metastatic gastric cancer), urothelial bladder carcinoma, lung cancer, triple-negative breast cancer, renal cell carcinoma, including clear cell RCC or papillary RCC, cervical cancer, or head and neck squamous cell carcinoma. In certain embodiments, the combination therapy may comprise a TGFβ inhibitor, e.g., Ab6, and a checkpoint inhibitor therapy (e.g., pembrolizumab). certain embodiments, the combination therapy may further comprise an additional agent, e.g., an additional checkpoint inhibitory and/or another chemotherapeutic agent. In certain embodiments, the combination therapy may be administered to immunotherapy-naïve patients (e.g., patients who have not previously received a checkpoint inhibitor therapy) diagnosed with a cancer that has not received FDA approval for treatment with a checkpoint inhibitor therapy. Such cancer may be a microsatellite-stable colorectal cancer or pancreatic cancer. In certain embodiments, the combination therapy may comprise a TGFβ inhibitor, e.g., Ab6, a checkpoint inhibitor therapy (e.g., pembrolizumab), and at least one chemotherapeutic agent (e.g., axitinib, paclitaxel, cisplatin, and/or 5-fluorouracil). In certain embodiments, the checkpoint inhibitor therapy may be pembrolizumab, nivolumab, and/or atezolizumab. In certain embodiments, the combination therapy is administered to patients who have cancers characterized as exhibiting an immune-excluded phenotype. In certain embodiments, additional analyses of a patient's cancer may be carried out to further inform treatment, and such analyses may use known cancer-specific markers including microsatellite instability levels, PD-1 and/or PD-L1 expression level, and/or the presence of mutations in known cancer driver genes such as EGFR, ALK, ROS1, BRAF. In certain embodiments, tumor PD-L1 expression may be used as a biomarker of therapeutic response.

In certain embodiments, the TGFβ inhibitor, e.g., Ab6, may be administered in an amount of about 3000 mg, 2400 mg, 2000 mg, 1600 mg, 800 mg, 240 mg, 80 mg, or less. In certain embodiments, the TGFβ inhibitor (e.g., Ab6) may be administered in an amount of about 3000 mg, 2400 mg, 2000 mg, 1600 mg, 800 mg, 240 mg, 80 mg, or less, e.g., at a frequency of once every six weeks, once every four weeks, once every three weeks, once every two weeks, wherein the TGFβ inhibitor (e.g., Ab6) is administered alone or in combination with a checkpoint inhibitor therapy, e.g., an anti-PD-(L)1 therapy. In certain embodiments, the TGFβ inhibitor (e.g., Ab6) may be administered alone at 3000 mg once every three weeks.

In certain embodiments, the TGFβ inhibitor (e.g., Ab6) may be administered alone at 3000 mg once every six weeks. In certain embodiments, the TGFβ inhibitor (e.g., Ab6) may be administered at 3000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every six weeks. In certain embodiments, the TGFβ inhibitor (e.g., Ab6) may be administered at 2400 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every six weeks. In certain embodiments, the TGFβ inhibitor (e.g., Ab6) may be administered at 2000 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every six weeks. In certain embodiments, the TGFβ inhibitor (e.g., Ab6) may be administered at 1600 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every six weeks. In certain embodiments, the TGFβ inhibitor (e.g., Ab6) may be administered at 800 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every six weeks. In certain embodiments, the TGFβ inhibitor (e.g., Ab6) may be administered at 240 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every six weeks. In certain embodiments, the TGFβ inhibitor (e.g., Ab6) may be administered at 80 mg once every six weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every six weeks. In certain embodiments, the TGFβ inhibitor (e.g., Ab6) may be administered at an amount of less than 80 mg once every six weeks, wherein the TGFβ inhibitor (e.g., Ab6) is administered in combination with an anti-PD-(L)1 therapy. In some embodiments, the TGFβ inhibitor (e.g., Ab6) may be administered every six weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every four weeks. In certain embodiments, the TGFβ inhibitor (e.g., Ab6) may be administered at 3000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every four weeks. In certain embodiments, the TGFβ inhibitor (e.g., Ab6) may be administered at 2400 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every four weeks. In certain embodiments, the TGFβ inhibitor (e.g., Ab6) may be administered at 2000 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every four weeks. In certain embodiments, the TGFβ inhibitor (e.g., Ab6) may be administered at 1600 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every four weeks. In certain embodiments, the TGFβ inhibitor (e.g., Ab6) may be administered at 800 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every four weeks. In certain embodiments, the TGFβ inhibitor (e.g., Ab6) may be administered at 240 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every four weeks. In certain embodiments, the TGFβ inhibitor (e.g., Ab6) may be administered at 80 mg once every four weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every four weeks. In certain embodiments, the TGFβ inhibitor (e.g., Ab6) may be administered at an amount of less than 80 mg once every four weeks, wherein the TGFβ inhibitor (e.g., Ab6) is administered in combination with an anti-PD-(L)1 therapy. In some embodiments, the TGFβ inhibitor (e.g., Ab6) may be administered every four weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every three weeks. In certain embodiments, the TGFβ inhibitor (e.g., Ab6) may be administered at 3000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every three weeks. In certain embodiments, the TGFβ inhibitor (e.g., Ab6) may be administered at 2400 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every three weeks. In certain embodiments, the TGFβ inhibitor (e.g., Ab6) may be administered at 2000 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every three weeks. In certain embodiments, the TGFβ inhibitor (e.g., Ab6) may be administered at 1600 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every three weeks. In certain embodiments, the TGFβ inhibitor (e.g., Ab6) may be administered at 800 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 800 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every three weeks. In certain embodiments, the TGFβ inhibitor (e.g., Ab6) may be administered at 240 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every three weeks. In certain embodiments, the TGFβ inhibitor (e.g., Ab6) may be administered at 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of three weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every three weeks. In certain embodiments, the TGFβ inhibitor (e.g., Ab6) may be administered at an amount of less than 80 mg once every three weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of three weeks, wherein the TGFβ inhibitor (e.g., Ab6) is administered in combination with an anti-PD-(L)1 therapy. In some embodiments, the TGFβ inhibitor (e.g., Ab6) may be administered every three weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) may be administered alone at 3000 mg once every two weeks. In certain embodiments, the TGFβ inhibitor (e.g., Ab6) may be administered at 3000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 3000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2400 mg once every two weeks. In certain embodiments, the TGFβ inhibitor (e.g., Ab6) may be administered at 2400 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2400 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 2000 mg once every two weeks. In certain embodiments, the TGFβ inhibitor (e.g., Ab6) may be administered at 2000 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 2000 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 1600 mg once every two weeks. In certain embodiments, the TGFβ inhibitor (e.g., Ab6) may be administered at 1600 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 1600 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 800 mg once every two weeks. In certain embodiments, the TGFβ inhibitor (e.g., Ab6) may be administered at 800 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 800 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 240 mg once every two weeks. In certain embodiments, the TGFβ inhibitor (e.g., Ab6) may be administered at 240 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 240 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at 80 mg once every two weeks. In certain embodiments, the TGFβ inhibitor (e.g., Ab6) may be administered at 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at 80 mg at a frequency of any multiples of two weeks, wherein the TGFβ inhibitor (e.g., Ab6) is administered in combination with an anti-PD-(L)1 therapy. In certain embodiments, the TGFβ inhibitor (e.g., Ab6) may be administered alone at an amount of less than 80 mg once every two weeks. In certain embodiments, the TGFβ inhibitor (e.g., Ab6) may be administered at an amount of less than 80 mg once every two weeks, wherein the TGFβ inhibitor (e.g., Ab6) is administered in combination with an anti-PD-(L)1 therapy. In certain embodiments, the TGFβ inhibitor (e.g., Ab6) may be administered at less than 80 mg at a frequency of any multiples of two weeks, wherein the TGFβ inhibitor (e.g., Ab6) is administered in combination with an anti-PD-(L)1 therapy. In some embodiments, the TGFβ inhibitor (e.g., Ab6) may be administered every two weeks at a dose of 50-3000 mg, e.g., 200-3000 mg, 200-1000 mg, 250-750 mg, 500-2000 mg, 750-2000 mg, 1000-2000 mg, 250-2500 mg, 1000-3000 mg, 1500-2500 mg, 2000-3000 mg.

In certain embodiments, the TGFβ inhibitor (e.g., Ab6) is administered in combination with pembrolizumab to a subject having NSCLC, UC, MEL, esophageal cancer, gastric cancer, HNSCC, HCC, cervical cancer, SCLC, PMBCL, Hodgkin's lymphoma, MSI-H or dMMR cancer, or TMB-H cancer. In certain embodiments, the subject is a non-responder to pembrolizumab. In certain embodiments, the subject has not received pembrolizumab previously. In certain embodiments, the TGFβ inhibitor (e.g., Ab6) is administered in combination with pembrolizumab to a subject having NSCLC who is a non-responder to pembrolizumab treatment. In certain embodiments, the subject having NSCLC has not received pembrolizumab previously. In certain embodiments, the TGFβ inhibitor (e.g., Ab6) is administered in combination with pembrolizumab to a subject having MEL who is a non-responder to pembrolizumab treatment. In certain embodiments, the subject having MEL has not received pembrolizumab previously. In certain embodiments, the TGFβ inhibitor (e.g., Ab6) is administered in combination with pembrolizumab to a subject having UC or mUC who is a non-responder to pembrolizumab treatment. In certain embodiments, the subject having UC or mUC has not received pembrolizumab previously.

In certain embodiments, the TGFβ inhibitor (e.g., Ab6) is administered in combination with nivolumab to a subject having NSCLC, UC, MEL, esophageal cancer, HNSCC, HCC, RCC, Hodgkin's lymphoma, MSI-H or dMMR CRC, or MPM. In certain embodiments, the subject is a non-responder to nivolumab. In certain embodiments, the subject has not received nivolumab previously.

In certain embodiments, the TGFβ inhibitor (e.g., Ab6) is administered in combination with cemiplimab to a subject having BCC or CSCC. In certain embodiments, the subject is a non-responder to cemiplimab. In certain embodiments, the subject has not received cemiplimab previously.

In certain embodiments, the TGFβ inhibitor (e.g., Ab6) is administered in combination with atezolizumab to a subject having NSCLC, MEL, HCC, TNBC, or SCLC. In certain embodiments, the subject is a non-responder to atezolizumab. In certain embodiments, the subject has not received atezolizumab previously.

In certain embodiments, the TGFβ inhibitor (e.g., Ab6) is administered in combination with avelumab to a subject having UC or MCC. In certain embodiments, the subject is a non-responder to avelumab. In certain embodiments, the subject has not received avelumab previously.

In certain embodiments, the TGFβ inhibitor (e.g., Ab6) is administered in combination with durvalumab to a subject having NSCLC or SCLC. In certain embodiments, the subject is a non-responder to durvalumab. In certain embodiments, the subject has not received durvalumab previously.

In certain embodiments, the TGFβ inhibitor (e.g., Ab6) is administered in combination with a checkpoint inhibitor therapy to a subject having a solid tumor for which a checkpoint inhibitor therapy has been approved. In certain embodiments, the subject has a tumor type that has been approved for treatment with a combination of a checkpoint inhibitor therapy and a chemotherapy. In certain embodiments, the subject has a tumor type that typically exhibits immune exclusion in more than 50% of the tumor area (e.g., tumor nests). In certain embodiments, the immune excluded tumor types include triple-negative breast cancer or renal cell carcinoma.

In certain embodiments, the TGFβ inhibitor (e.g., Ab6) is administered in combination with a checkpoint inhibitor therapy to a subject having a solid tumor for which a checkpoint inhibitor monotherapy has been approved. In certain embodiments, the subject has a tumor type that typically exhibits immune exclusion in more than 50% of the tumor area (e.g., tumor nests), such as non-small cell lung cancer, urothelial carcinoma, gastric cancer, and renal cell carcinoma. In certain embodiments, the subject has a tumor type that typically exhibits immune exclusion in less than 50% of the tumor area (e.g., tumor nests), such as small-cell lung cancer or melanoma.

In certain embodiments, the TGFβ inhibitor (e.g., Ab6) is administered in combination with a checkpoint inhibitor therapy to a subject having a solid tumor for which a checkpoint inhibitor has not been approved. In certain embodiments, the subject has a tumortype that typically exhibits immune exclusion in more than 50% of the tumor area (e.g., tumor nests), such as microsatellite stable colorectal cancer, pancreatic cancer, and prostate cancer. Without wishing to be bound by theory, it is possible that tumor types that do not have an approved checkpoint inhibitor therapy respond poorly to checkpoint inhibitor therapy with or without a conventional chemotherapy. Thus, it is contemplated that such tumor types, particularly tumors exhibiting immune exclusion, may have better response to checkpoint inhibitor therapy when combined with a TGFβ inhibitor therapy (e.g., Ab6).

Thus, TGFβ inhibitors (e.g., Ab6) may be used in conjunction (e.g., in combination) with a checkpoint inhibitor therapy for the treatment of cancer in a subject, wherein the cancer comprises an immunosuppressive tumor, and wherein the immunosuppressive tumor is resistant to checkpoint inhibitor therapy. Non-limiting examples of immunosuppressive tumors include acute myeoid leukemia, adrenocortical cancer, brain lower grade glioma, cholangiocarcinoma, colon adenocarcinoma, diffuse large B-cell lymphoma, esophageal carcinoma, glioblastoma multiforme, kidney chromophobe, lung squamous cell carcinoma, mesothelioma, ovarian serous cystadenocarcinoma, pheochromocytoma and paraganglioma, prostate adenocarcinoma, rectum adenocarcinoma, sarcoma, testicular germ cell tumor, thymoma, thyroid carcinoma, uterine carcinosarcoma, uterine corpus endometrioid carcinoma, or uveal melanoma.

In some embodiments, the at least one additional agent binds a T-cell costimulation molecule, such as inhibitory costimulation molecules and activating costimulation molecules. In some embodiments, the at least one additional agent is selected from the group consisting of an anti-CD40 antibody, an anti-CD38 antibody, an anti-KIR antibody, an anti-CD33 antibody, an anti-CD137 antibody, and an anti-CD74 antibody.

In some embodiments, the at least one additional therapy is radiation. In some embodiments, the at least one additional agent is a radiotherapeutic agent. In some embodiments, the at least one additional agent is a chemotherapeutic agent. In some embodiments, the chemotherapeutic agent is Taxol. In some embodiments, the at least one additional agent is an anti-inflammatory agent. In some embodiments, the at least one additional agent inhibits the process of monocyte/macrophage recruitment and/or tissue infiltration. In some embodiments, the at least one additional agent is an inhibitor of hepatic stellate cell activation. In some embodiments, the at least one additional agent is a chemokine receptor antagonist, e.g., CCR2 antagonists and CCR5 antagonists. In some embodiments, such chemokine receptor antagonist is a dual specific antagonist, such as a CCR2/CCR5 antagonist. In some embodiments, the at least one additional agent to be administered as combination therapy is or comprises a member of the TGFβ superfamily of growth factors or regulators thereof. In some embodiments, such agent is selected from modulators (e.g., inhibitors and activators) of GDF8/myostatin and GDF11. In some embodiments, such agent is an inhibitor of GDF8/myostatin signaling. In some embodiments, such agent is a monoclonal antibody that specifically binds a pro/latent myostatin complex and blocks activation of myostatin. In some embodiments, the monoclonal antibody that specifically binds a pro/latent myostatin complex and blocks activation of myostatin does not bind free, mature myostatin; see, for example, WO 2017/049011.

In some embodiments, an additional therapy comprises cell therapy, such as CAR-T therapy and CAR-NK therapy.

In some embodiments, an additional therapy comprises administering an anti-VEGF therapy, such as a VEGF inhibitor, e.g., bevacizumab. In some embodiments, inhibitors of TGFβ contemplated herein may be used in conjunction with (e.g., combination therapy, add-on therapy, etc.) a VEGF inhibitor (e.g., bevacizumab) for the treatment of solid cancer (e.g., ovarian cancer). In some embodiments, inhibitors of TGFβ contemplated herein may be used in conjunction with (e.g., combination therapy, add-on therapy, etc.) a VEGF inhibitor (e.g., bevacizumab) for the treatment of hematopoietic cancers.

In some embodiments, an additional therapy is a cancer vaccine. Numerous clinical trials that tested peptide-based cancer vaccines have targeted hematological malignancies (cancers of the blood), melanoma (skin cancer), breast cancer, head and neck cancer, gastroesophageal cancer, lung cancer, pancreatic cancer, prostate cancer, ovarian cancer, and colorectal cancers. The antigens included peptides from HER2, telomerase (TERT), survivin (BIRC5), and Wilms' tumor 1 (WT1). Several trials also used “personalized” mixtures of 12-15 distinct peptides. That is, they contain a mixture of peptides from the patient's tumor that the patient exhibits an immune response against. Some trials are targeting solid tumors, glioma, glioblastoma, melanoma, and breast, cervical, ovarian, colorectal, and non-small lung cell cancers and include antigens from MUC1, IDO1 (Indoleamine 2,3-dioxygenase), CTAG1 B, and two VEGF receptors, FLT1 and KDR. Notably, the IDO1 vaccine is tested in patients with melanoma in combination with the immune checkpoint inhibitor ipilimumab and the BRAF (gene) inhibitor vemurafenib.

Non-limiting examples of tumor antigens useful as cancer vaccines include: NY-ESO-1, HER2, HPV16 E7 (Papillomaviridae #E7), CEA (Carcinoembryonic antigen), WT1, MART-1, gp100, tyrosinase, URLC10, VEGFR1, VEGFR2, surviving, MUC1 and MUC2.

Activated immune cells primed by such cancer vaccine may, however, be excluded from the TME in part through TGFβ31-dependent mechanisms. To overcome the immunosuppression, use of TGFβ1 inhibitors of the present disclosure may be considered so as to unleash the potential of the vaccine.

Combination therapies contemplated herein may advantageously utilize lower dosages of the administered therapeutic agents, thus avoiding possible toxicities or complications associated with the various monotherapies. In some embodiments, use of an isoform-specific inhibitor of TGFβ1 described herein may render those who are poorly responsive or not responsive to a therapy (e.g., standard of care) more responsive. In some embodiments, use of an isoform-specific inhibitor of TGFβ1 described herein may allow reduced dosage of the therapy (e.g., standard of care) which still produces equivalent clinical efficacy in patients but fewer or lesser degrees of drug-related toxicities or adverse events.

In some embodiments, inhibitors of TGFβ contemplated herein may be used in conjunction with (e.g., combination therapy, add-on therapy, etc.) a selective inhibitor of myostatin (GDF8). In some embodiments, the selective inhibitor of myostatin is an inhibitor of pro/latent myostatin activation. See, for example, the antibodies disclosed in WO 2017/049011, such as apitegromab.

### Advantages of TGFβ1 Inhibitors as a Therapeutic

It has been recognized that various diseases involve heterogeneous populations of cells as sources of TGFβ1 that collectively contribute to the pathogenesis and/or progression of the disease. More than one types of TGFβ31-containing complexes (“contexts”) likely coexist within the same disease microenvironment. In particular, such diseases may involve both an ECM (or “matrix”) component of TGFβ1 signaling (e.g., ECM dysregulation) and an immune component of TGFβ1 signaling. In such situations, selectively targeting only a single TGFβ1 context (e.g., TGFβ1 associated with one particular type of presenting molecule) may provide limited relief. Thus, broadly inhibitory TGFβ1 antagonists are desirable for therapeutic use. Previously described inhibitory antibodies that broadly targeted multiple latent complexes of TGFβ1 exhibited skewed binding profiles among the target complexes (see, for example, WO 2018/129329 and WO 2019/075090). The inventors therefore set out to identify more uniformly inhibitory antibodies that selectively inhibit TGFβ1 activation, irrespective of particular presenting molecule linked thereto. It was reasoned that particularly for immune-oncology applications, it is advantageous to potently inhibit both matrix-associated TGFβ1 and immune cell-associated TGFβ1.

In various embodiments, context-independent inhibitors of TGFβ1 are used in the treatments and methods disclosed herein to target the pro/latent forms of TGFβ1. More specifically, in one modality, the inhibitor targets ECM-associated TGFβ1 (LTBP1/3-TGFβ1 complexes). In another modality, the inhibitor targets immune cell-associated TGFβ1. This includes GARP-presented TGFβ1, such as GARP-TGFβ1 complexes expressed on Treg cells and LRRC33-TGFβ1 complexes expressed on macrophages and other myeloid/lymphoid cells, as well as certain cancer cells.

Such antibodies may include isoform-specific inhibitors of TGFβ1 that bind and prevent activation (or release) of mature TGFβ1 growth factor from a pro/latent TGFβ1 complex in a context-independent manner, such that the antibodies can inhibit activation (or release) of TGFβ1 associated with multiple types of presenting molecules. In particular, the present disclosure provides antibodies capable of blocking ECM-associated TGFβ1 (LTBP-presented and LTBP3-presented complexes) and cell-associated TGFβ1 (GARP-presented and LRRC33-presented complexes).

Various disease conditions have been suggested to involve dysregulation of TGFβ signaling as a contributing factor. Indeed, the pathogenesis and/or progression of certain human conditions appear to be predominantly driven by or dependent on TGFβ1 activities. In particular, many such diseases and disorders involve both an ECM component and an immune component of TGFβ1 function, suggesting that TGFβ1 activation in multiple contexts (e.g., mediated by more than one type of presenting molecules) is involved. Moreover, it is contemplated that there is crosstalk among TGFβ1-responsive cells. In some cases, interplays between multifaceted activities of the TGFβ1 axis may trigger a cascade of events that lead to disease progression, aggravation, and/or suppression of the host's ability to combat disease. For example, certain disease microenvironments, such as tumor microenvironment (TME) and fibrotic microenvironment (FME), may be associated with TGFβ1 presented by multiple different presenting molecules, e.g., LTBP1-proTGFβ1, LTBP3-proTGFβ1, GARP-proTGFβ1, LRRC33-proTGFβ1, and any combinations thereof. TGFβ1 activities of one context may in turn regulate or influence TGFβ1 activities of another context, raising the possibility that when dysregulated, this may result in exacerbation of disease conditions. Therefore, it is desirable to broadly inhibit across multiple modes of TGFβ1 function (i.e., multiple contexts) while selectively limiting such inhibitory effects to the TGFβ1 isoform. The aim is not to perturb homeostatic TGFβ signaling mediated by the other isoforms, including TGFβ3, which plays an important role in would healing.

Immune components of TGFβ1 activities are largely mediated by cell-associated TGFβ1 (e.g., GARP-proTGFβ1 and LRRC33-proTGFβ1). Both the GARP- and LRRC33-arms of TGFβ1 function are associated with immunosuppressive features that contribute to the progression of many diseases. Thus, TGFβ inhibitors such as the TGFβ1 inhibitors described herein, may be used to inhibit TGFβ1 associated with immunosuppressive cells. The immunosuppressive cells include regulatory T-cells (Tregs), M2 macrophages/tumor-associated macrophages, and MDSCs. The TGFβ inhibitors of the current disclosure may inhibit, reduce, or reverse immunosuppressive phenotype at a disease site such as the tumor microenvironment.

In some embodiments, the TGFβ1 inhibitor inhibits TGFβ1 associated with a cell expressing the GARP-TGFβ1 complex or the LRRC33-TGFβ1 complex, wherein optionally the cell may be a T-cell, a fibroblast, a myofibroblast, a macrophage, a monocyte, a dendritic cell, an antigen presenting cell, a neutrophil, a myeloid-derived suppressor cell (MDSC), a lymphocyte, a mast cell, or a microglial cell. The T-cell may be a regulatory T cell (e.g., immunosuppressive T cell). The neutrophil may be an activated neutrophil. The macrophage may be an activated (e.g., polarized) macrophage, including profibrotic and/or tumor-associated macrophages (TAM), e.g., M2c subtype and M2d subtype macrophages. In some embodiments, macrophages are exposed to tumor-derived factors (e.g., cytokines, growth factors, etc.) which may further induce pro-cancer phenotypes in macrophages. In some embodiments, such tumor-derived factor is CSF-1/M-CSF.

In some embodiments, the cell expressing the GARP-TGFβ1 complex or the LRRC33-TGFβ1 complex is a cancer cell, e.g., circulating cancer cells and tumor cells.

### TGFβ Inhibitors Useful for Carrying Out the Compositions and Methods of the Disclosure

TGFβ inhibitors suitable for the therapeutic use and related methods disclosed herein include small molecule (i.e., low molecular weight) antagonists and biologics. Such inhibitors include isoform-selective inhibitors and isoform-non-selective inhibitors. Biologics inhibitors include antibodies, antigen-binding fragments thereof, antibody-based or immunoglobulin-like molecules, as well as other engineered constructs, typically fusion proteins, such as ligand traps. Ligand traps typically include a ligand-binding moiety that is derived from ligand-binding portion or portions of TGFβ receptor(s). Such biologics may be multifunctional constructs, such as bi-functional fusion proteins and bispecific antibodies.

In some embodiments, methods disclosed herein may employ one or more of the following: low molecular weight antagonists of TGFβ receptors, e.g., ALK5 antagonists, such as Galunisertib (LY2157299 monohydrate); monoclonal antibodies (such as neutralizing antibodies) that inhibit all three isoforms (“pan-inhibitor” antibodies) (see, for example, WO 2018/134681); monoclonal antibodies that preferentially inhibit two of the three isoforms (e.g., antibodies against TGFβ1/2 (for example WO 2016/161410) and TGFβ1/3 (for example WO 2006/116002); and engineered molecules (e.g., fusion proteins) such as ligand traps (for example, WO 2018/029367; WO 2018/129331 and WO 2018/158727). In some embodiments, methods disclosed herein may employ one or more of the TGFβ inhibitors disclosed in Batlle and Massague (Immunity, 2019. Apr. 16; 50(4):924-940), the content of which is incorporated herein in its entirety.

In some embodiments, the TGFβ inhibitor is a TGFβ1-selective inhibitor, such as a TGFβ1-selective inhibitor disclosed herein, e.g., Ab6. In some embodiments, the TGFβ1-selective inhibitor is one disclosed in PCT/US2019/041390, PCT/US2019/041373, or PCT/US2021/012930, the contents of each of which are hereby incorporated by reference in their entirety.

In some embodiments, the low molecular weight antagonists of TGFβ receptors may include Vactosertib (TEW-7197, EW-7197), LY3200882, PF-06952229, AZ 12601011, and/or AZ 12799734.

In some embodiments, the neutralizing pan-TGFβ antibody is GC1008, also known as fresolimumab, or a derivative thereof. In some embodiments, such antibody comprises the sequence in accordance with the disclosure of WO/2018/134681. In some embodiments, the pan-TGFβ antibody is SAR439459 or a derivative thereof.

In some embodiments, the TGFβ1/2 antibodies include XPA-42-089 or a derivative thereof.

In some embodiments, the antibody is a neutralizing antibody that specifically binds both TGFβ1 and TGFβ3. In some embodiments such antibody preferentially binds TGFβ1 over TGFβ3. For example, the antibody comprises the sequence in accordance with the disclosure of WO/2006/116002. In some embodiments, the antibody is 21D1.

In some embodiments, the antibody is a neutralizing antibody that specifically binds both TGFβ1 and TGFβ2. In some embodiments, the antibody comprises the sequence in accordance with the disclosure of WO/2016/161410. In some embodiments, the antibody is XOMA-089, or NIS-793.

In some embodiments, the antibody is an activation inhibitor antibody that is selective for TGFβ1. In some embodiments, the antibody comprises the sequence in accordance with the disclosure of WO/2015/015003, WO/2019/075090 or WO/2016/115345.

In some embodiments, the antibody is a neutralizing antibody that is selective for TGFβ1. In some embodiments, the antibody comprises the sequence in accordance with the disclosure of WO/2013/134365 or WO/2018/043734.

In some embodiments, the TGFβ inhibitor is a ligand trap. In some embodiments, the ligand trap comprises the structure in accordance with the disclosure of WO/2018/158727. In some embodiments, the ligand trap comprises the structure in accordance with the disclosure of WO 2018/029367; WO 2018/129331. In some embodiments, the ligand trap is a construct known as CTLA4− TGFbRII. In some embodiments, the ligand trap is a bi-functional fusion protein comprising a checkpoint inhibitor function and a TGFβ inhibitor function. In some embodiments, the bi-functional fusion protein is a construct known as M7824 or PDL1-TGFbRII. In some embodiments, the TGFβ inhibitor is a receptor based TGFβ trap, e.g., AVID200.

In some embodiments, the TGFβ inhibitor is an integrin inhibitor. In some embodiments, the TGFβ inhibitor is an inhibitor of an integrin such as αVβ1, αVβ3, αVβ5, αVβ6, αVβ8, α5β1, αIIbβ3, and/or α8β1. Integrin inhibitors include small molecule inhibitors and antibodies that bind to an integrin and/or inhibit the binding of an integrin to the RGD motif of proTGFβ1 and/or proTGFβ3.

In some embodiments, the TGFβ inhibitor is an inhibitor of latent TGFβ (e.g., latent TGFβ1 or latent TGFβ3). In some embodiments, the TGFβ inhibitor is an inhibitor that binds the RGD motif of proTGFβ1 and/or proTGFβ3.

In some embodiments, the TGFβ inhibitor is an LTBP-selective inhibitor, such as an LTBP-selective inhibitor disclosed in PCT/US2020/015915. In some embodiments, the TGFβ inhibitor is an LTBP1-selective inhibitor. In some embodiments, the TGFβ inhibitor is an LTBP3-selective inhibitor. In some embodiments, the TGFβ inhibitor is an LTBP-1 and LTBP3-selective inhibitor.

Isoform-Selective Antibodies of proTGFβ1

Preferably, the therapeutic use and related methods in accordance with the present disclosure are carried out with an isoform-selective inhibitor of TGFβ1, e.g., Ab6 (the sequence of which is as disclosed in PCT/US2019/041373, the contents of which are herein incorporated by reference in its entirety).

Applicant previously disclosed improved antibodies which embody all or most of the following features: 1) selectivity towards TGFβ1 is maintained to minimize unwanted toxicities associated with pan-inhibition (“isoform-selectivity”) (see, for example, PCT/US2017/021972); 2) exhibit broad binding activities across various biological contexts, or, both matrix-associated and cell-associated categories (“context-independent”) (see, for example, WO 2018/129329); 3) achieve more even or unbiased affinities across multiple antigen complexes (“uniformity”); 4) show strong binding activities for each of the antigen complexes, (“high-affinity”) and have robust inhibitory activities for each context (“potency”) (see, for example, PCT/US2019/041373); and, 5) the preferred mechanism of action is to inhibit the activation step so the inhibitor can target a tissue-tethered, latent TGFβ1 complex, so as to preemptively prevent downstream activation events to achieve durable effects, rather than to directly target soluble/free growth factors (“durability”). As disclosed in PCT/US2019/041373, such TGFβ1 inhibitors are highly potent and highly selective inhibitor of latent TGFβ1 activation. Data presented therein demonstrated, inter alia, that this mechanism of isoform-selective inhibition is sufficient to overcome primary resistance to anti-PD-1 in syngeneic mouse models that closely recapitulate some of the features of primary resistance to CBT found in human cancers. In addition, 6) such inhibitors have an improved safety profile as compared to pan-inhibitors or other isoform-non-selective inhibitors of TGFβ, Together, these efficacy and safety data provide a rationale for exploring the therapeutic use of selective TGFβ1 inhibition to broaden and enhance clinical responses to checkpoint blockade in cancer immunotherapy, as well as to treat a number of additional TGFβ31-related indications.

### General Features of Certain TGF61 Inhibitors

Exemplary antibodies that may be used for carrying out the present disclosure are disclosed in WO2020014460, the content of which is incorporated herein by reference in its entirety.

Preferred antibodies and corresponding nucleic acid sequences that encode such antibodies useful for carrying out the present disclosure include one or more of the CDR amino acid sequences shown in Tables 1 and 2. Each set of the H-CDRs (H-CDR1, H-CDR2 and H-CDR3) listed in Table 1 can be combined with the L-CDRs (L-CDR1, L-CDR2 and L-CDR3) provided in Table 2.

Thus, the disclosure provides an antibody or antigen binding fragment thereof, e.g., an isolated antibody or antigen-binding fragment thereof, comprising six CDRs (e.g., an H-CDR1, an H-CDR2, an H-CDR3, an L-CDR1, an L-CDR2 and an L-CDR3) as listed in Table 1, and wherein the L-CDR1 comprises QASQDITNYLN (SEQ ID NO: 78), the L-CDR2 comprises DASNLET (SEQ ID NO: 79), and the L-CDR3 comprises QQADNHPPWT (SEQ ID NO: 6), wherein optionally, the H-CDR1 may comprise FTFSSFSMD (SEQ ID NO: 80); the H-CDR-2 may comprise YISPSADTIYYADSVKG (SEQ ID NO: 76); and/or, the H-CDR3 may comprise ARGVLDYGDMLMP (SEQ ID NO: 3). In some embodiments, the antibody or the fragment comprises H-CDR1 having the amino acid sequence FTFSSFSMD (SEQ ID NO: 80), H-CDR2 having the amino acid sequence YISPSADTIYYADSVKG (SEQ ID NO: 76), and H-CDR-3 having the amino acid sequence ARGVLDYGDMLMP (SEQ ID NO: 3); L-CDR1 having the amino acid sequence QASQDITNYLN (SEQ ID NO: 78), L-CDR2 having the amino acid sequence DASNLET (SEQ ID NO: 79), and L-CDR3 having the amino acid sequence QQADNHPPWT (SEQ ID NO: 6).

Additional examples of antibodies or antigen-binding fragments thereof encompassed by the present disclosure are provided in PCT/US2019/041390, PCT/US2019/041373, PCT/US2021/012930, the contents of each of which are herein incorporated by reference in their entirety.

TABLE 1
Complementary determining regions of the heavy chain of exemplary antibodies,
as determined using the numbering scheme described in Lu et al.

| Ab | H-CDR1 | H-CDR2 | H-CDR3 |
|---|---|---|---|
| Ab4 | FTFSSYSMN | YISSSSSTIYYADSVKG | ARGVLDYGDMLDP |
| | (SEQ ID NO: 81) | (SEQ ID NO: 82) | (SEQ ID NO: 83) |
| Ab5 | FTFSSFSMD | YISPDASTIYYADSVKG | ARGVLDYGDMLDP |
| | (SEQ ID NO: 80) | (SEQ ID NO: 84) | (SEQ ID NO: 83) |
| Ab6 | FTFSSFSMD | YISPSADTIYYADSVKG | ARGVLDYGDMLMP |
| | (SEQ ID NO: 80) | (SEQ ID NO: 76) | (SEQ ID NO: 3) |
| Ab21 | FTFSSFSMD | YISPDASTIYYADSVKG | ARGVLDYGDMLDP |
| | (SEQ ID NO: 80) | (SEQ ID NO: 84) | (SEQ ID NO: 83) |
| Ab22 | FTFGSFSMN | YIHSDASTIYYADSVKG | ARGVLDYGDMLDP |
| | (SEQ ID NO: 88) | (SEQ ID NO: 86) | (SEQ ID NO: 83) |
| Ab23 | FTFSSFSMN | YISPSADTIYYADSVKG | ARGVLDYGDMLDP |
| | (SEQ ID NO: 87) | (SEQ ID NO: 76) | (SEQ ID NO: 83) |
| Ab24 | FTFSSFAMY | YISPDASTIYYADSVKG | ARGVLDYGDMLDP |
| | (SEQ ID NO: 179) | (SEQ ID NO: 84) | (SEQ ID NO: 83) |
| Ab25 | FTFGSFSMD | YISPDASTIYYADSVKG | ARGVLDYGDMLDP |
| | (SEQ ID NO: 180) | (SEQ ID NO: 84) | (SEQ ID NO: 83) |
| Ab26 | FTFSSFSMD | YISPDASTIYYADSVKG | ARGVLDYGDMLDP |
| | (SEQ ID NO: 80) | (SEQ ID NO: 84) | (SEQ ID NO: 83) |
| Ab27 | FTFSFYAMN | YISPDASTIYYADSVKG | ARGVLDYGDMLDP |
| | (SEQ ID NO: 90) | (SEQ ID NO: 84) | (SEQ ID NO: 83) |
| Ab28 | FTFSSFSMD | YISPDASTIYYADSVKG | VRGVLDYGDMLDP |
| | (SEQ ID NO: 80) | (SEQ ID NO: 84) | (SEQ ID NO: 91) |
| Ab29 | FTFSSFAMN | YISPDASTIYYAGSVKG | VRAVLDYGDMLDP |
| | (SEQ ID NO: 92) | (SEQ ID NO: 93) | (SEQ ID NO: 94) |
| Ab30 | FTFSSFSMD | YISPDASTIYYADSVKG | ARGTLDYGDMLDP |
| | (SEQ ID NO: 80) | (SEQ ID NO: 84) | (SEQ ID NO: 95) |
| Ab31 | FTFSSFSMD | YISPDASTIYYADSVKG | ARAVLDYGDMLDP |
| | (SEQ ID NO: 80) | (SEQ ID NO: 84) | (SEQ ID NO: 96) |
| Ab32 | FTFSSFSMN | YISPSADTIYYADSVKG | ARGVWDMGDMLDP |
| | (SEQ ID NO: 87) | (SEQ ID NO: 76) | (SEQ ID NO: 97) |
| Ab33 | FTFSSFSMN | YISPSADTIYYADSVKG | AHGVLDYGDMLDP |
| | (SEQ ID NO: 87) | (SEQ ID NO: 76) | (SEQ ID NO: 98) |
| Ab34 | FTFAFYSMN | YISPDASTIYYADSVKG | ARGVLDYGDMLDP |
| | (SEQ ID NO: 99) | (SEQ ID NO: 84) | (SEQ ID NO: 83) |

TABLE 2
Complementary determining regions of the light
chain of exemplary antibodies, as determined
using the Kabat numbering scheme or the
numbering system of Lu et al.

| L-CDR1 | L-CDR2 | L-CDR3 |
|---|---|---|
| QASQDITNYLN | DASNLET | QQADNHPPWT |
| (SEQ ID NO: 78) | (SEQ ID NO: 79) | (SEQ ID NO: 6) |

Determination of CDR sequences within an antibody depends on the particular numbering scheme being employed. Commonly used systems include but are not limited to: Kabat numbering system, IMTG numbering system, Chothia numbering system, and others such as the numbering scheme described by Lu et al., (Lu X et al., MAbs. 2019 January; 11(1):45-57). To illustrate, 6 CDR sequences ofAb6 as defined by four different numbering systems are exemplified below. Any art-recognized CDR numbering systems may be used to define CDR sequences of the antibodies of the present disclosure.

TABLE 3
Six CDRs of an exemplary antibody (Ab6) based on four numbering schemes

| | IMTG numbering | Kabat numbering | Chothia numbering | System of Lu et al. |
|---|---|---|---|---|
| H-CDR1 | GFTFSSFS | SFSMD | GFTFSSF | FTFSSFSMD |
| | (SEQ ID NO: 1) | (SEQ ID NO: 75) | (SEQ ID NO: 168) | (SEQ ID NO: 80) |
| H-CDR2 | ISPSADTI | YISPSADTIYYADSVKG | SPSADT | YISPSADTIYYADSVKG |
| | (SEQ ID NO: 2) | (SEQ ID NO: 76) | (SEQ ID NO: 169) | (SEQ ID NO: 76) |
| H-CDR3 | ARGVLDYGDMLMP | GVLDYGDMLMP | GVLDYGDMLMP | ARGVLDYGDMLMP |
| | (SEQ ID NO: 3) | (SEQ ID NO: 77) | (SEQ ID NO: 77) | (SEQ ID NO: 3) |
| L-CDR1 | QDITNY | QASQDITNYLN | QASQDITNYLN | QASQDITNYLN |
| | (SEQ ID NO: 4) | (SEQ ID NO: 78) | (SEQ ID NO: 78) | (SEQ ID NO: 78) |
| L-CDR2 | DAS | DASNLET | DASNLET | DASNLET |
| | (SEQ ID NO: 5) | (SEQ ID NO: 79) | (SEQ ID NO: 79) | (SEQ ID NO: 79) |
| L-CDR3 | QQADNHPPWT | QQADNHPPWT | QQADNHPPWT | QQADNHPPWT |
| | (SEQ ID NO: 6) | (SEQ ID NO: 6) | (SEQ ID NO: 6) | (SEQ ID NO: 6) |

Amino acid sequences of the heavy chain variable domain and the light chain variable domain of exemplary antibodies of the present disclosure are provided in Table 4. Thus, in some embodiments, the isoform-selective TGFβ1 inhibitor of the present disclosure may be an antibody or an antigen-binding fragment thereof comprising a heavy chain variable domain (VH) and a light chain variable domain (VW), wherein the VH and the VL sequences are selected from any one of the sets of VH and VL sequences listed in Table 4 below.

TABLE 4
Heavy chain variable domains and light chain variable domains of exemplary antibodies

| | Heavy Chain Variable Domain (VH) | Light Chain Variable Domain (VL) |
|---|---|---|
| Ab4 | EVQLVESGGGLVQPGGSLRLSCAASGFTFSS | DIQMTQSPSSLSASVGDRVTITCQASQDITNYLN |
| | YSMNWVRQAPGKGLEWVSYISSSSSTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 100) | (SEQ ID NO: 8) |
| Ab5 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 101) | (SEQ ID NO: 8) |
| Ab6 | EVQLVESGGGLVQPGGSLRLSCTASGFTFSS | DIQMTQSPSSLSASVGDRVTITCQASQDITNYLN |
| | FSMDWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVLDYGDMLMPWGQGTLVTVSS | GTKVEIK |
| | (SEQ ID NO: 7) | (SEQ ID NO: 8) |
| Ab21 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 102) | (SEQ ID NO: 8) |
| Ab22 | EVQLVESGGGLVQPGGSLRLSCAASGFTFGS | DIQMTQSPSSLSASVGDRVTITCQASQDITNYLN |
| | FSMNWVRQAPGKGLEWVSYIHSDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 103) | (SEQ ID NO: 8) |
| Ab23 | EVQLVESGGGLVQPGGSLRLSCAASGFTFSS | DIQMTQSPSSLSASVGDRVTITCQASQDITNYLN |
| | FSMNWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 104) | (SEQ ID NO: 8) |
| Ab24 | EVQLVESGGGLVQPGRSLRLSCAASGFTFSS | DIQMTQSPSSLSASVGDRVTITCQASQDITNYLN |
| | FAMYWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 105) | (SEQ ID NO: 8) |
| Ab25 | EVQLVESGGGLVQPGGSLRLSCAASGFTFGS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 106) | (SEQ ID NO: 8) |
| Ab26 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 107) | (SEQ ID NO: 8) |
| Ab27 | EVQLVESGGGLVQPGGSLRLSCAASGFTFSF | DIQMTQSPSSLSASVGDRVTITCQASQDITNYLN |
| | YAMNWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 108) | (SEQ ID NO: 8) |
| Ab28 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCVRGVLDYGDMLDPWGQGTLVTVSS | GTKVEIK |
| | (SEQ ID NO: 109) | (SEQ ID NO: 8) |
| Ab29 | EVQLVESGGGLVQPGRSLRLSCAASGFTFSS | DIQMTQSPSSLSASVGDRVTITCQASQDITNYLN |
| | FAMNWVRQAPGKGLEWVSYISPDASTIYYAG | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCVRAVLDYGDMLDPWGQGTLVTVSS | GTKVEIK |
| | (SEQ ID NO: 110) | (SEQ ID NO: 8) |
| Ab30 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGTLDYGDMLDPWGQGTLVTVSS | GTKVEIK |
| | (SEQ ID NO: 111) | (SEQ ID NO: 8) |
| Ab31 | EVQLVESGGGLVQPGGSLRLSCAASGFTFSS | DIQMTQSPSSLSASVGDRVTITCQASQDITNYLN |
| | FSMDWVRQAPGKGLEWVSYISPDASTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARAVLDYGDMLDPWGQGTLVTVSS | GTKVEIK |
| | (SEQ ID NO: 112) | (SEQ ID NO: 8) |
| Ab32 | EVQLVESGGGLVQPGGSLRLSCAASGFTFSS | DIQMTQSPSSLSASVGDRVTITCQASQDITNYLN |
| | FSMNWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCARGVWDMGDMLDPWGQGTLVTVSS | GTKVEIK |
| | (SEQ ID NO: 113) | (SEQ ID NO: 8) |
| Ab33 | EVQLVESGGGLVQPGGSLRLSCAASGFTFSS | DIQMTQSPSSLSASVGDRVTITCQASQDITNYLN |
| | FSMNWVRQAPGKGLEWVSYISPSADTIYYAD | WYQQKPGKAPKLLIYDASNLETGVPSRFSGSGS |
| | SVKGRFTISRDNAKNTLYLQMNSLRAEDTAVY | GTDFTFTISSLQPEDIATYYCQQADNHPPWTFGG |
| | YCAHGVLDYGDMLDPWGQGTLVTVSS | GTKVEIK |
| | (SEQ ID NO: 114) | (SEQ ID NO: 8) |
| Ab34 | EVQLVESGGGLVQPGGSLRLSCAASGFTFAF | DIQMTQSPSSLSASVGDRVTITCQASQDITNYLN |
| | YSMNWVRQAPGKGLEWVSYISPDASTIYYAD | WYQQKPGKAPKLLIYDASNLETGVPSRFSGSGS |
| | SVKGRFTISRDNAKNSLYLQMNSLRAEDTAVY | GTDFTFTISSLQPEDIATYYCQQADNHPPWTFGG |
| | YCARGVLDYGDMLDPWGQGTLVTVSS | GTKVEIK |
| | (SEQ ID NO: 115) | (SEQ ID NO: 8) |

In some embodiments, an antibody or an antigen-binding fragment thereof is disclosed that comprises a heavy chain variable domain and a light chain variable domain, wherein, the heavy chain variable domain has at least 90% (e.g., at least 90%, 91%, 92%, 93%, 94%, 95%, 96%, 97%, 98% 99% and 100%) sequence identity with any one of the sequences selected from the group consisting of: Ab4, Ab5, Ab6, Ab21, Ab22, Ab23, Ab24, Ab25, Ab26, Ab27, Ab28, Ab29, Ab30, Ab31, Ab32, Ab33, and Ab34; and, wherein the light chain variable domain has at least 90% identity with any one of the sequences selected from Ab4, Ab5, Ab6, Ab21, Ab22, Ab23, Ab24, Ab25, Ab26, Ab27, Ab28, Ab29, Ab30, Ab31, Ab32, Ab33, and Ab34, wherein, optionally, the heavy chain variable domain may optionally have at least 95% sequence identity, and/or, the light chain variable domain may have at least 95% (e.g., at least 95%, 96%, 97%, 98% 99% and 100%) sequence identity. In some embodiments, the heavy chain variable domain of the antibody or the fragment has at least 90% sequence identity with SEQ ID NO: 7, and wherein optionally, the light chain variable domain of the antibody or the fragment has at least 90% sequence identity with SEQ ID NO: 8. In some embodiments, the heavy chain variable domain of the antibody or the fragment has at least 95% sequence identity with SEQ ID NO: 7, and wherein optionally, the light chain variable domain of the antibody or the fragment has at least 95% sequence identity with SEQ ID NO: 8. In some embodiments, the heavy chain variable domain of the antibody or the fragment has at least 98% sequence identity with SEQ ID NO: 7, and wherein optionally, the light chain variable domain of the antibody or the fragment has at least 98% sequence identity with SEQ ID NO: 8. In some embodiments, the heavy chain variable domain of the antibody or the fragment has 100% sequence identity with SEQ ID NO: 7, and wherein optionally, the light chain variable domain of the antibody or the fragment has 100% sequence identity with SEQ ID NO: 8.

In various embodiments, an antibody or an antigen-binding fragment thereof disclosed herein comprises 6 CDRs from, or the full sequences of, the heavy and light chain variable domains of SEQ ID Nos: 7 and 8, respectively. In some embodiments, the antibody or an antigen-binding fragment thereof comprises heavy and light chain variable domain sequences with at least 90% sequence identity (e.g., at least 95% identity) to SEQ ID NOs: 7 and 8, respectively. For instance, the antibody or an antigen-binding fragment thereof may comprise a set of 6 respective H- and L-CDRs selected from those set out in Tables 1 and 2 above. In some certain embodiments, the antibody or antigen-binding fragment thereof comprises a set of 6 respective H- and L-CDRs as set out in Table 3 (e.g., using the system of Lu et al.).

Alternatively, or in addition, the antibody or an antigen-binding fragment thereof used in the context of the present disclosure may comprise heavy and light chain variable domains with at least 90% sequence identity (e.g., at least 95% identity) to SEQ ID Nos: 7 and 8, respectively, and specifically binds a proTGFβ1 complex at (i) a first binding region comprising at least a portion of Latency Lasso (SEQ ID NO: 126); and ii) a second binding region comprising at least a portion of Finger-1 (SEQ ID NO: 124); characterized in that when bound to the proTGFβ1 complex in a solution, the antibody or the fragment protects the binding regions from solvent exposure as determined by hydrogen-deuterium exchange mass spectrometry (HDX-MS). The first binding region may comprise PGPLPEAV (SEQ ID NO: 134) or a portion thereof and the second binding region may comprise RKDLGWKW (SEQ ID NO: 143) or a portion thereof. As used herein, protection of the binding region refers to protein-protein interactions, such as antibody-antigen binding, the degree by which a protein (e.g., a region of a protein containing an epitope) is exposed to a solvent as assessed by an HDX-MS-based assay of protein-protein interactions. Protection of binding may be determined by the level of proton exchange occurring at a binding site, which is inversely correlates with the degree of binding/interaction. Therefore, when an antibody described herein binds to a region of an antigen, the binding region is “protected” from being exposed to the solvent because the protein-protein interaction precludes the binding region from being accessible by the surrounding solvent. The protected region is thus indicative of a site of interaction. The antibody or the fragment may further bind the proTGFβ1 complex at one or more of the following binding regions or a portion thereof:

(SEQ ID NO: 132)
LVKRKRIEA;
(SEQ ID NO: 126)
LASPPSQGEVPPGPL;
(SEQ ID NO: 135)
LALYNSTR;
(SEQ ID NO: 136)
REAVPEPVL;
(SEQ ID NO: 137)
YQKYSNNSWR;
(SEQ ID NO: 144)
RKDLGWKWIHE;
(SEQ ID NO: 145)
HEPKGYHANF;
(SEQ ID NO: 139)
LGPCPYIWS;
(SEQ ID NO: 140)
ALEPLPIV;
and,
(SEQ ID NO: 141)
VGRKPKVEQL.

In some embodiments, the antibody or antigen-binding fragments may further be characterized in that it cross-blocks (cross-competes) for binding to TGFβ1 (e.g., to pro- and/or latent-TGFβ1) with an antibody having the heavy chain variable domain of SEQ ID NO: 7, and the light chain variable domain of SEQ ID NO: 8. In some embodiments, the antibody that cross-blocks or cross-competes comprises heavy and light chain variable domains that are at least about 90% (e.g., 95% or 99%) identical to those of SEQ ID NOs 7 and 8, respectively.

In some embodiments, the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises a heavy chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 7, and a light chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 8. In some embodiments, the antibody or antigen binding portion thereof, comprises a heavy chain variable domain amino acid sequence encoded by the nucleic acid sequence set forth in SEQ ID NO: 7, and a light chain variable domain amino acid sequence encoded by the nucleic acid sequence set forth in SEQ ID NO: 8.

In some examples, any of the antibodies of the disclosure that specifically bind to a GARP-TGFβ1 complex, a LTBP1-TGFβ1 complex, a LTBP3-TGFβ1 complex, and/or a LRRC33-TGFβ1 complex include any antibody (including antigen binding portions thereof) having one or more CDR (e.g., CDRH or CDRL) sequences substantially similar to CDRH1, CDRH2, CDRH3, CDRL1, CDRL2, and/or CDRL3. For example, the antibodies may include one or more CDR sequences as shown in Table 1 containing up to 5, 4, 3, 2, or 1 amino acid residue variations as compared to the corresponding CDR region in any one of SEQ ID NOs: 3, 6, 76, 78, 79, 80, 81, 82, 83 84, 85, 86, 87, 88, 89, 90, 91, 92, 93, 94, 95, 96, 97, 98, and 99. In some embodiments, one or more of the six CDR sequences contain up to three (3) amino acid changes as compared to the sequences provided in Table 1. Such antibody variants comprising up to 3 amino acid changes per CDR are encompassed by the present disclosure. In some embodiments, such variant antibodies are generated by the process of optimization, such as affinity maturation. The complete amino acid sequences for the heavy chain variable region and light chain variable region of the antibodies listed in Table 4 (e.g., Ab6), as well as nucleic acid sequences encoding the heavy chain variable region and light chain variable region of certain antibodies are provided below:

Ab6-Heavy chain variable region amino acid sequence
(SEQ ID NO: 7)
EVQLVESGGGLVQPGGSLRLSCTASGFTFSSFSMDWVRQAPGKGLEWVSYISPSADTIYYADSVKGRFTISRDN
AKNTLYLQMNSLRAEDTAVYYCARGVLDYGDMLMPWGQGTLVTVSS
Ab6-Light chain variable region amino acid sequence
(SEQ ID NO: 8)
DIQMTQSPSSLSASVGDRVTITCQASQDITNYLNWYQQKPGKAPKLLIYDASNLETGVPSRFSGSGSGTDFTFTIS
SLQPEDIATYYCQQADNHPPWTFGGGTKVEIK
Ab6-Heavy chain amino acid sequence
(SEQ ID NO: 9)
EVQLVESGGGLVQPGGSLRLSCTASGFTFSSFSMDWVRQAPGKGLEWVSYISPSADTIYYADSVKGRFTISRDN
AKNTLYLQMNSLRAEDTAVYYCARGVLDYGDMLMPWGQGTLVTVSSASTKGPSVFPLAPCSRSTSESTAALGC
LVKDYFPEPVTVSWNSGALTSGVHTFPAVLQSSGLYSLSSVVTVPSSSLGTKTYTCNVDHKPSNTKVDKRVESK
YGPPCPPCPAPEFLGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEALHNHYTQKSLSL
SLG
Ab6-Heavy chain nucleic acid sequence
(SEQ ID NO: 10)
GAGGTGCAGCTGGTGGAGTCTGGGGGAGGCTTGGTACAGCCTGGGGGGTCCCTGAGACTCTCCTGTACAG
CCTCTGGATTCACCTTCAGTAGCTTCAGCATGGACTGGGTCCGCCAGGCTCCAGGGAAGGGGCTGGAGTG
GGTTTCATACATTAGTCCCAGTGCAGACACCATATACTACGCAGACTCTGTGAAGGGCCGATTCACCATCTC
CAGAGACAATGCCAAGAACACGCTGTATCTGCAAATGAACAGCCTGAGAGCCGAGGACACGGCGGTGTACT
ACTGCGCCAGAGGGGTGCTCGACTACGGAGACATGTTAATGCCATGGGGCCAGGGAACCCTGGTCACCGT
CTCCTCAGCGTCGACCAAGGGCCCTTCCGTGTTCCCTCTGGCCCCTTGCTCCCGGTCCACCTCCGAGTCCA
CCGCCGCTCTGGGCTGTCTGGTGAAGGACTACTTCCCTGAGCCTGTGACCGTGAGCTGGAACTCTGGCGC
CCTGACCTCCGGCGTGCACACCTTCCCTGCCGTGCTGCAGTCCTCCGGCCTGTACTCCCTGTCCTCCGTGG
TGACCGTGCCTTCCTCCTCCCTGGGCACCAAGACCTACACCTGCAACGTGGACCACAAGCCTTCCAACACC
AAGGTGGACAAGCGGGTGGAGTCCAAGTACGGCCCTCCTTGCCCTCCCTGCCCTGCCCCTGAGTTCCTGG
GCGGACCCTCCGTGTTCCTGTTCCCTCCTAAGCCTAAGGACACCCTGATGATCTCCCGGACCCCTGAGGTG
ACCTGCGTGGTGGTGGACGTGTCCCAGGAAGATCCTGAGGTCCAGTTCAATTGGTACGTGGATGGCGTGG
AGGTGCACAACGCCAAGACCAAGCCTCGGGAGGAACAGTTCAACTCCACCTACCGGGTGGTGTCTGTGCT
GACCGTGCTGCACCAGGACTGGCTGAACGGCAAGGAATACAAGTGCAAGGTCAGCAACAAGGGCCTGCCC
TCCTCCATCGAGAAAACCATCTCCAAGGCCAAGGGCCAGCCTCGCGAGCCTCAGGTGTACACCCTGCCTCC
TAGCCAGGAAGAGATGACCAAGAATCAGGTGTCCCTGACATGCCTGGTGAAGGGCTTCTACCCTTCCGATA
TCGCCGTGGAGTGGGAGAGCAACGGCCAGCCAGAGAACAACTACAAGACCACCCCTCCTGTGCTGGACTC
CGACGGCTCCTTCTTCCTGTACTCCAGGCTGACCGTGGACAAGTCCCGGTGGCAGGAAGGCAACGTCTTTT
CCTGCTCCGTGATGCACGAGGCCCTGCACAACCACTACACCCAGAAGTCCCTGTCCCTGTCTCTGGGC
Ab6-Light chain amino acid sequence
(SEQ ID NO: 11)
DIQMTQSPSSLSASVGDRVTITCQASQDITNYLNWYQQKPGKAPKLLIYDASNLETGVPSRFSGSGSGTDFTFTIS
SLQPEDIATYYCQQADNHPPWTFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
Ab6-Light chain nucleic acid sequence (human kappa)
(SEQ ID NO: 12)
GACATCCAGATGACCCAGTCTCCATCCTCCCTGTCTGCATCTGTAGGAGACAGAGTCACCATCACTTGCCAG
GCGAGTCAGGACATTACCAACTATTTAAATTGGTATCAGCAGAAACCAGGGAAAGCCCCTAAGCTCCTGATC
TACGATGCATCCAATTTGGAAACAGGGGTCCCATCAAGGTTCAGTGGAAGTGGATCTGGGACAGATTTTACT
TTCACCATCAGCAGCCTGCAGCCTGAAGATATTGCAACATATTACTGTCAGCAGGCCGACAATCACCCTCCT
TGGACTTTTGGCGGAGGGACCAAGGTTGAGATCAAACGTACGGTGGCTGCACCATCTGTCTTCATCTTCCC
GCCATCTGATGAGCAGTTGAAATCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGAGAAACACAA
AGTCTACGCCTGCGAAGTCACCCATCAGGGCCTGAGCTCGCCCGTCACAAAGAGCTTCAACAGGGGAGAG
TGT

In some embodiments, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al., J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In any of the antibodies or antigen-binding fragments described herein, one or more conservative mutations can be introduced into the CDRs or framework sequences at positions where the residues are not likely to be involved in an antibody-antigen interaction. In some embodiments, such conservative mutation(s) can be introduced into the CDRs or framework sequences at position(s) where the residues are not likely to be involved in interacting with a GARP-TGFβ1 complex, a LTBP1-TGFβ1 complex, a LTBP3-TGFβ1 complex, and a LRRC33-TGFβ1 complex as determined based on the crystal structure. In some embodiments, likely interface (e.g., residues involved in an antigen-antibody interaction) may be deduced from known structural information on another antigen sharing structural similarities.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, NewYork, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like (CPPCP (SEQ ID NO: 43)) hinge sequence. Accordingly, any of the antibodies may include a stabilizing ‘Adair’ mutation or the amino acid sequence CPPCP (SEQ ID NO: 43).

Isoform-specific, context-independent inhibitors of TGFβ1 of the present disclosure may optionally comprise antibody constant regions or parts thereof. For example, a VL domain may be attached at its C-terminal end to a light chain constant domain like CK or CA. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

Additionally or alternatively, such antibodies may or may not include the framework region of the antibodies of SEQ ID NOs: 100, 101, 102, 103, 104, 105, 106, 107, 108, 109, 110, 111, 112, 113, 114, 115, and 8. In some embodiments, antibodies that specifically bind to a GARP-TGFβ1 complex, a LTBP1-TGFβ1 complex, a LTBP3-TGFβ1 complex, and a LRRC33-TGFβ1 complex are murine antibodies and include murine framework region sequences.

In some embodiments, such antibodies bind to a GARP-TGFβ1 complex, a LTBP1-TGFβ1 complex, a LTBP3-TGFβ1 complex, and a LRRC33-TGFβ1 complex with relatively high affinity, e.g., with a K_{D} less than 10-9 M, 10⁻¹⁰ M, 10-11 M or lower. For example, such antibodies may bind a GARP-TGFβ1 complex, a LTBP1-TGFβ1 complex, a LTBP3-TGFβ1 complex, and/or a LRRC33-TGFβ1 complex with an affinity between 5 pM and 1 nM, e.g., between 10 pM and 1 nM, e.g., between 10 pM and 500 pM. The disclosure also includes antibodies or antigen binding fragments that compete with any of the antibodies described herein for binding to a GARP-TGFβ1 complex, a LTBP1-TGFβ1 complex, a LTBP3-TGFβ1 complex, and/or a LRRC33-TGFβ1 complex and that have a KD value of 1 nM or lower (e.g., 1 nM or lower, 500 pM or lower, 100 pM or lower). The affinity and binding kinetics of the antibodies that specifically bind to a GARP-TGFβ1 complex, a LTBP1-TGFβ1 complex, a LTBP3-TGFβ1 complex, and/or a LRRC33-TGFβ1 complex can be tested using any suitable method including but not limited to biosensor-based technology (e.g., OCTET® or Biacore®) and solution equilibrium titration-based technology (e.g., MSD-SET). In some embodiments, affinity and binding kinetics are measured by SPR, such as Biacore systems. In preferred embodiments, such antibodies dissociate from each of the aforementioned large latent complex with an OFF rate of 10e-4 or less.

In some embodiments, inhibitors of cell-associated TGFβ1 (e.g., GARP-presented TGFβ1 and LRRC33-presented TGFβ1) according to the disclosure include antibodies or fragments thereof that specifically bind such complex (e.g., GARP-pro/latent TGFβ1 and LRRC33-pro/latent TGFβ1) and trigger internalization of the complex. This mode of action causes removal or depletion of the inactive TGFβ1 complexes (e.g., GARP-proTGFβ1 and LRRC33-proTGFβ1) from the cell surface (e.g., Treg, macrophages, etc.), hence reducing TGFβ1 available for activation. In some embodiments, such antibodies or fragments thereof bind the target complex in a pH-dependent manner such that binding occurs at a neutral or physiological pH, but the antibody dissociates from its antigen at an acidic pH; or, dissociation rates are higher at acidic pH than at neutral pH. Such antibodies or fragments thereof may function as recycling antibodies.

Antibodies Competing with the Preferred Antibodies of TGFβ1

Aspects of the disclosure relate to antibodies that compete or cross-compete with any of the antibodies provided herein. The term “compete”, as used herein with regard to an antibody, means that a first antibody binds to an epitope (e.g., an epitope of a GARP-proTGFβ1 complex, a LTBP1-proTGFβ1 complex, a LTBP3-proTGFβ1 complex, and a LRRC33-proTGFβ1 complex) in a manner sufficiently similar to or overlapping with the binding of a second antibody, such that the result of binding of the first antibody with its epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its epitope or ligand, whether to the same, greater, or lesser extent, the antibodies are said to “cross-compete” with each other for binding of their respective epitope(s). Both competing and cross-competing antibodies are within the scope of this disclosure. Regardless of the mechanism by which such competition or cross-competition occurs (e.g., steric hindrance, conformational change, or binding to a common epitope, or portion thereof), the skilled artisan would appreciate that such competing and/or cross-competing antibodies are encompassed and can be useful for the methods and/or compositions provided herein. The term “cross-blocking” and “cross-competing” may be used interchangeably.

Two different monoclonal antibodies (or antigen-binding fragments) that bind the same antigen may be able to simultaneously bind to the antigen if the binding sites are sufficiently further apart in the three-dimensional space such that each binding does not interfere with the other binding. By contrast, two different monoclonal antibodies may have binding regions of an antigen that are the same or overlapping, in which case, binding of the first antibody may prevent the second antibody from being able to bind the antigen, or vice versa. In the latter case, the two antibodies are said to “cross-block” with each other with respect to the same antigen.

Antibody “binning” experiments are useful for classifying multiple antibodies that are made against the same antigen into various “bins” based on the relative cross-blocking activities. Each “bin” therefore represents a discrete binding region(s) of the antigen. Antibodies in the same bin by definition cross-block each other. Binning can be examined by standard in vitro binding assays, such as Biacore or Octet®, using standard test conditions, e.g., according to the manufacturer's instructions (e.g., binding assayed at room temperature, ˜20-25° C.).

Aspects of the disclosure relate to antibodies that compete or cross-compete with any of the specific antibodies, or antigen binding portions thereof, as provided herein. In some embodiments, an antibody, or antigen binding portion thereof, binds at or near the same epitope as any of the antibodies provided herein. In some embodiments, an antibody, or antigen binding portion thereof, binds near an epitope if it binds within 15 or fewer amino acid residues of the epitope. In some embodiments, any of the antibody, or antigen binding portion thereof, as provided herein, binds within 1, 2, 3, 4, 5, 6, 7, 8, 9, 10, 11, 12, 13, 14 or 15 amino acid residues of an epitope that is bound by any of the antibodies provided herein.

In another embodiment, provided herein is an antibody, or antigen binding portion thereof, competes or cross-competes for binding to any of the antigens provided herein (e.g., a GARP-TGFβ1 complex, a LTBP1-TGFβ1 complex, a LTBP3-TGFβ1 complex, and/or a LRRC33-TGFβ1 complex) with an equilibrium dissociation constant, K_{D}, between the antibody and the protein of less than 10-8 M. In other embodiments, an antibody competes or cross-competes for binding to any of the antigens provided herein with a K_{D} in a range from 10⁻¹² M to 10-9 M. In some embodiments, provided herein is an anti-TGFβ1 antibody, or antigen binding portion thereof that competes for binding with an antibody, or antigen binding portion thereof, described herein. In some embodiments, provided herein is an anti-TGFβ1 antibody, or antigen binding portion thereof, that binds to the same epitope as an antibody, or antigen binding portion thereof, described herein.

Any of the antibodies provided herein can be characterized using any suitable methods. For example, one method is to identify the epitope to which the antigen binds, or “epitope mapping.” There are many suitable methods for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). In some embodiments, the epitope is a TGFβ1 epitope that is only available for binding by the antibody, or antigen binding portion thereof, described herein, when the TGFβ1 is in a GARP-proTGFβ1 complex, a LTBP1-proTGFβ1 complex, a LTBP3-proTGFβ1 complex, or a LRRC33-proTGFβ1 complex. Peptides of varying lengths (e.g., at least 4-6 amino acids long) can be isolated or synthesized (e.g., recombinantly) and used for binding assays with an antibody. In another example, the epitope to which the antibody binds can be determined in a systematic screen by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the GARP-proTGFβ1 complex, a LTBP1-proTGFβ1 complex, a LTBP3-proTGFβ1 complex, and/or a proLRRC33-TGFβ1 complex have been replaced (swapped) with sequences from a closely related, but antigenically distinct protein, such as another member of the TGFβ protein family (e.g., GDF11).

Alternatively, competition assays can be performed using other antibodies known to bind to the same antigen to determine whether an antibody binds to the same epitope as the other antibodies. Competition assays are well known to those of skill in the art.

In some embodiments, a pharmaceutical composition may be made by a process comprising a step of: selecting an antibody or antigen-binding fragment thereof, which cross-competes with an antibody having a heavy chain variable domain of SEQ ID NO: 7 and a light chain variable domain of SEQ ID NO: 8 for binding to TGFβ1 (e.g., to pro-TGFβ1 and/or latent TGFβ1).

In some embodiments, a pharmaceutical composition may be made by the process comprising a step of: selecting an antibody or antigen-binding fragment thereof, which cross-competes with the antibody selected from the group consisting of Ab4, Ab5, Ab6, Ab21, Ab22, Ab23, Ab24, Ab25, Ab26, Ab27, Ab28, Ab29, Ab30, Ab31, Ab32, Ab33 and Ab34; and, formulating into a pharmaceutical composition.

Preferably, the antibody selected by the process is a high-affinity binder characterized in that the antibody or the antigen-binding fragment is capable of binding to each of human LLCs (e.g., hLTBP1-proTGFβ1, hLTBP3-proTGFβ1, hGARP-proTGFβ1 and hLRRC33-proTGFβ1) with a K_{D} of ≤1 nM, as measured by solution equilibrium titration. Such cross-competing antibodies may be used in the treatment of TGFβ31-related indications a subject in accordance with the present disclosure.

### Various Modifications and Variations of Antibodies

Non-limiting variations, modifications, and features of any of the antibodies or antigen-binding fragments thereof encompassed by the present disclosure are briefly discussed below. Embodiments of related analytical methods are also provided.

Naturally-occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human antibody light chains are typically classified as kappa and lambda light chains. Heavy chains are typically classified as mu, delta, gamma, alpha, or epsilon, and define the isotype of the antibody. An antibody can be of any type (e.g., IgM, IgD, IgG, IgA, IgY, and IgE) and class (e.g., IgG₁, IgG₂, IgG₃, IgG₄, IgM₁, IgM₂, IgA₁, and IgA₂). Within full-length light and heavy chains, typically, the variable and constant regions are joined by a “J” region of about 12 or more amino acids, with the heavy chain also including a “D” region of about 10 more amino acids (see, e.g., Fundamental Immunology, Ch. 7 (Paul, W., ed., 2nd ed. Raven Press, N.Y. (1989)) (incorporated by reference in its entirety)). The variable regions of each light/heavy chain pair typically form the antigen binding site.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk (1987) J. Mol. Biol. 196: 901-917; Chothia et al., (1989) Nature 342: 878-883. The CDRs of a light chain can also be referred to as CDR-L1, CDR-L2, and CDR-L3, and the CDRs of a heavy chain can also be referred to as CDR-H1, CDR-H2, and CDR-H3. In some embodiments, an antibody can comprise a small number of amino acid deletions from the carboxy end of the heavy chain(s). In some embodiments, an antibody comprises a heavy chain having 1-5 amino acid deletions in the carboxy end of the heavy chain. In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In some embodiments, various methods of analysis can be employed to identify or approximate the CDR regions. Examples of such methods include, but are not limited to, the Kabat definition, the Chothia definition, the AbM definition, the definition described by Lu et al (see above), and the contact definition.

An “affinity matured” antibody is an antibody with one or more alterations in one or more CDRs thereof, which result in an improvement in the affinity of the antibody for antigen compared to a parent antibody, which does not possess those alteration(s). Exemplary affinity matured antibodies will have nanomolar or even picomolar affinities (e.g., K_{D} of ˜10⁻⁹ M-10⁻¹² M range) for the target antigen. Affinity matured antibodies are produced by procedures known in the art. Marks et al., (1992) Bio/Technology 10: 779-783 describes affinity maturation by VH and VL domain shuffling. Random mutagenesis of CDR and/or framework residues is described by Barbas, et al., (1994) Proc Nat. Acad. Sci. USA 91: 3809-3813; Schier et al., (1995) Gene 169: 147-155; Yelton et al., (1995) J. Immunol. 155: 1994-2004; Jackson et al., (1995) J. Immunol. 154(7): 3310-9; and Hawkins et al., (1992) J. Mol. Biol. 226: 889-896; and selective mutation at selective mutagenesis positions, contact or hypermutation positions with an activity enhancing amino acid residue is described in U.S. Pat. No. 6,914,128. Typically, a parent antibody and its affinity-matured progeny (e.g., derivatives) retain the same binding region within an antigen, although certain interactions at the molecular level may be altered due to amino acid residue alternation(s) introduced by affinity maturation.

The term “CDR-grafted antibody” refers to antibodies, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “chimeric antibody” refers to antibodies, which comprise heavy and light chain variable region sequences from one species and constant region sequences from another species, such as antibodies having murine heavy and light chain variable regions linked to human constant regions.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

In some embodiments, the antibody or antigen-binding fragment thereof comprises a heavy chain framework region 1 (H-FR1) having the following amino acid sequence with optionally 1, 2 or 3 amino acid changes: EVQLVESGGGLVQPGGSLRLSCAASG (SEQ ID NO: 147). For example, the Gly residue at position 16 may be replaced with an Arg (R); and/or, the Ala residue at position 23 may be replaced with a Thr (T).

In some embodiments, the antibody or antigen-binding fragment thereof comprises a heavy chain framework region 2 (H-FR2) having the following amino acid sequence with optionally 1, 2 or 3 amino acid changes: WVRQAPGKGLEWVS (SEQ ID NO: 148).

In some embodiments, the antibody or antigen-binding fragment thereof comprises a heavy chain framework region 3 (H-FR3) having the following amino acid sequence with optionally 1, 2 or 3 amino acid changes: RFTISRDNAKNSLYLQMNSLRAEDTAVYYC (SEQ ID NO: 149). For example, the Ser residue at position 12 may be replaced with a Thr (T).

In some embodiments, the antibody or antigen-binding fragment thereof comprises a heavy chain framework region 4 (H-FR4) having the following amino acid sequence with optionally 1, 2 or 3 amino acid changes: WGQGTLVTVSS (SEQ ID NO: 150).

In some embodiments, the antibody or antigen-binding fragment thereof comprises a light chain framework region 1 (L-FR1) having the following amino acid sequence with optionally 1, 2 or 3 amino acid changes: DIQMTQSPSSLSASVGDRVTITC (SEQ ID NO: 151).

In some embodiments, the antibody or antigen-binding fragment thereof comprises a light chain framework region 2 (L-FR2) having the following amino acid sequence with optionally 1, 2 or 3 amino acid changes: WYQQKPGKAPKLLIY (SEQ ID NO: 152).

In some embodiments, the antibody or antigen-binding fragment thereof comprises a light chain framework region 3 (L-FR3) having the following amino acid sequence with optionally 1, 2 or 3 amino acid changes: GVPSRFSGSGSGTDFTFTISSLQPEDIATYYC (SEQ ID NO: 153).

In some embodiments, the antibody or antigen-binding fragment thereof comprises a light chain framework region 4 (L-FR4) having the following amino acid sequence with optionally 1, 2 or 3 amino acid changes: FGGGTKVEIK (SEQ ID NO: 154).

In some embodiments, the antibody, or antigen binding portion thereof, comprises a heavy chain immunoglobulin constant domain of a human IgM constant domain, a human IgG constant domain, a human IgG1 constant domain, a human IgG2 constant domain, a human IgG2A constant domain, a human IgG2B constant domain, a human IgG2 constant domain, a human IgG3 constant domain, a human IgG3 constant domain, a human IgG4 constant domain, a human IgA constant domain, a human IgA1 constant domain, a human IgA2 constant domain, a human IgD constant domain, or a human IgE constant domain. In some embodiments, the antibody, or antigen binding portion thereof, comprises a heavy chain immunoglobulin constant domain of a human IgG1 constant domain or a human IgG4 constant domain. In some embodiments, the antibody, or antigen binding portion thereof, comprises a heavy chain immunoglobulin constant domain of a human IgG4 constant domain. In some embodiments, the antibody, or antigen binding portion thereof, comprises a heavy chain immunoglobulin constant domain of a human IgG4 constant domain having a backbone substitution of Ser to Pro that produces an IgG1-like hinge and permits formation of inter-chain disulfide bonds.

In some embodiments, the antibody or antigen binding portion thereof, further comprises a light chain immunoglobulin constant domain comprising a human Ig lambda constant domain or a human Ig kappa constant domain.

In some embodiments, the antibody is an IgG having four polypeptide chains which are two heavy chains and two light chains.

In some embodiments, wherein the antibody is a humanized antibody, a diabody, or a chimeric antibody. In some embodiments, the antibody is a humanized antibody. In some embodiments, the antibody is a human antibody. In some embodiments, the antibody comprises a framework having a human germline amino acid sequence.

In some embodiments, the antigen binding portion is a Fab fragment, a F(ab′)2 fragment, a scFab fragment, or an scFv fragment.

In some embodiments, the antibody contains one or more amino acid modifications in the Fc region. In some embodiments, modifications in the Fc region may provide altered properties, such as altered half life in circulation, e.g., by altering affinity for Fc receptors such as FcRn (Front Immunol. 2019 Jun. 7; 10:1296). In some embodiments, modifications to the Fc region of the antibody provides increased FcγR binding. In some embodiments, modifications in the Fc region provide improved antibody effector function. In some embodiments, modifications in the Fc region provide increased in vivo half-life for the antibody. In some embodiments, the half life of the antibody may be increased by increasing its affinity for binding to FcRn at low pH (e.g., pH<6.5). In some embodiments, such Fc modifications may include Met252Tyr, Ser254Thr, and Thr256Glu substitutions (see, e.g., U.S. Pat. No. 7,083,784).

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin (see, e.g., Shapiro et al., (2002) Crit. Rev. Immunol. 22(3): 183-200; Marchalonis et al., (2001) Adv. Exp. Med. Biol. 484: 13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “neutralizing” refers to counteracting the biological activity of an antigen (e.g., target protein) when a binding protein specifically binds to the antigen. In an embodiment, the neutralizing binding protein binds to the antigen/target, e.g., cytokine, kinase, growth factor, cell surface protein, soluble protein, phosphatase, or receptor ligand, and reduces its biologically activity by at least about 20%, 40%, 60%, 80%, 85%, 90%, 95%, 96%, 97%, 98%, 99% or more. In some embodiments, a neutralizing antibody to a growth factor specifically binds a mature, soluble growth factor that has been released from a latent complex, thereby preventing its ability to bind its receptor to elicit downstream signaling. In some embodiments, the mature growth factor is TGFβ1 or TGFβ3. The term “binding protein” as used herein includes any polypeptide that specifically binds to an antigen (e.g., TGFβ1), including, but not limited to, an antibody, or antigen binding portions thereof, and a bispecific or multispecific construct that comprises an antigen binding region (e.g., a region capable of binding TGFβ1) and a region capable of binding one or more additional antigens or additional epitopes on a single antigen. Examples include a DVD-Ig™, a TVD-Ig, a RAb-Ig, a bispecific antibody, and a dual specific antibody. A binding protein may also comprise an antibody-drug conjugate, e.g., wherein a second agent (e.g., a small molecule checkpoint inhibitor) is linked to an antibody or antigen-binding fragment thereof capable of binding TGFβ1 (e.g., capable of binding pro- and/or latent-TGFβ1)

The term “monoclonal antibody” or “mAb” when used in a context of a composition comprising the same may refer to an antibody preparation obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “recombinant human antibody,” as used herein, is intended to include all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. R. (1997) TIB Tech. 15: 62-70; Azzazy, H. and Highsmith, W. E. (2002) Clin. Biochem. 35: 425-445; Gavilondo, J. V. and Larrick, J. W. (2002) BioTechniques 29: 128-145; Hoogenboom, H. and Chames, P. (2000) Immunol. Today 21: 371-378, incorporated herein by reference), antibodies isolated from an animal (e.g., a mouse) that is transgenic for human immunoglobulin genes (see, Taylor, L. D. et al., (1992) Nucl. Acids Res. 20: 6287-6295; Kellermann, S-A. and Green, L. L. (2002) Cur. Opin. in Biotechnol. 13: 593-597; Little, M. et al., (2000) Immunol. Today 21: 364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “Dual Variable Domain Immunoglobulin” or “DVD-Ig™” and the like include binding proteins comprising a paired heavy chain DVD polypeptide and a light chain DVD polypeptide with each paired heavy and light chain providing two antigen binding sites. Each binding site includes a total of 6 CDRs involved in antigen binding per antigen binding site. A DVD-Ig™ is typically has two arms bound to each other at least in part by dimerization of the CH3 domains, with each arm of the DVD being bispecific, providing an immunoglobulin with four binding sites. DVD-Ig™ are provided in US Patent Publication Nos. 2010/0260668 and 2009/0304693, each of which are incorporated herein by reference including sequence listings.

As used herein, “Triple Variable Domain Immunoglobulin” or “TVD-Ig” and the like are binding proteins comprising a paired heavy chain TVD binding protein polypeptide and a light chain TVD binding protein polypeptide with each paired heavy and light chain providing three antigen binding sites. Each binding site includes a total of 6 CDRs involved in antigen binding per antigen binding site. A TVD binding protein may have two arms bound to each other at least in part by dimerization of the CH3 domains, with each arm of the TVD binding protein being trispecific, providing a binding protein with six binding sites.

As used herein, “Receptor-Antibody Immunoglobulin” or “RAb-Ig” and the like are binding proteins comprising a heavy chain RAb polypeptide, and a light chain RAb polypeptide, which together form three antigen binding sites in total. One antigen binding site is formed by the pairing of the heavy and light antibody variable domains present in each of the heavy chain RAb polypeptide and the light chain RAb polypeptide to form a single binding site with a total of 6 CDRs providing a first antigen binding site. Each the heavy chain RAb polypeptide and the light chain RAb polypeptide include a receptor sequence that independently binds a ligand providing the second and third “antigen” binding sites. A RAb-Ig typically has two arms bound to each other at least in part by dimerization of the CH3 domains, with each arm of the RAb-Ig being trispecific, providing an immunoglobulin with six binding sites. RAb-Igs are described in US Patent Application Publication No. 2002/0127231, the entire contents of which including sequence listings are incorporated herein by reference).

In various embodiments, the present disclosure provides, in part, novel antibodies and antigen-binding fragments that may be used alone, linked to one or more additional agents (e.g., as ADCs), or as part of a larger macromolecule (e.g., a bispecific antibody, dual-specific antibody, or as a multispecific antibody, or as part of a construct further comprising a ligand trap, e.g., in combination with a TGFB ligand trap such as M7824 (Merck) and AVID200 (Forbius)), or as part of a bifunctional or multifunctional engineered construct (e.g., fusion proteins and ligand traps) and may be administered as part of pharmaceutical compositions or combination therapies.

The term “bispecific antibody,” as used herein, and as differentiated from a “bispecific half-Ig binding protein” or “bispecific (half-Ig) binding protein”, refers to full-length antibodies that are generated by quadroma technology (see Milstein, C. and Cuello, A. C. (1983) Nature 305(5934): p. 537-540), by chemical conjugation of two different monoclonal antibodies (see Staerz, U. D. et al., (1985) Nature 314(6012): 628-631), or by knob-into-hole or similar approaches, which introduce mutations in the Fc region that do not inhibit CH3-CH3 dimerization (see Holliger, P. et al., (1993) Proc. Natl. Acad. Sci USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen it binds to. For example, a bispecific antibody comprising two binding arms directed toward TGFβ1 and PD-1 may be used to combine a TGFβ1 inhibitor (Ab6 or Ab6-derived binding moiety) and a checkpoint inhibitor (e.g., an anti-PD1 antibody or moiety). Such a bispecific antibody may be used as an exemplary form of treatment for patients selected to receive a TGFβ1 inhibitor and checkpoint inhibitor combination therapy.

The term “dual-specific antibody,” as used herein, and as differentiated from a bispecific half-Ig binding protein or bispecific binding protein, refers to full-length antibodies that can bind two different antigens (or epitopes) in each of its two binding arms (a pair of HC/LC) (see PCT Publication No. WO 02/02773). Accordingly, a dual-specific binding protein has two identical antigen binding arms, with identical specificity and identical CDR sequences, and is bivalent for each antigen to which it binds.

The term “multispecific antibody” refers to an antibody or antigen binding fragment that displays binding specificity for two or more epitopes, where each binding site differs and recognizes a different epitope (on the same or different antigens). A bispecific antibody is an exemplary type of multispecific antibody. Higher order multispecifics (i.e., antibodies exhibiting more than two specificities) include but are not limited to trispecific antibodies in TriMAb, triple body, and tribody formats. For exemplary types of multispecific antibodies and/or methods of generating the same, see, e.g., Castoldi et al., Protein Eng Des Sel 2012; 25:551-9; Schubert et al., MAbs 2011; 3:21-30; Kugler et al., Br J Haematol 2010; 150:574-86; Schoonjans et al., J Immunol 2000; 165:7050-7; and Egan et al., MAbs 2017; 9(1):68-84, which are all incorporated herein by reference for such types and methods.

The term “Kₒₙ,” as used herein, is intended to refer to the on rate constant for association of a binding protein (e.g., an antibody) to the antigen to form the, e.g., antibody/antigen complex as is known in the art. The “Kₒₙ” also is known by the terms “association rate constant,” or “ka,” as used interchangeably herein. This value indicating the binding rate of an antibody to its target antigen or the rate of complex formation between an antibody and antigen also is shown by the equation: Antibody (“Ab”)+Antigen (“Ag”)→Ab-Ag.

The term “Koff,” as used herein, is intended to refer to the off rate constant for dissociation of a binding protein (e.g., an antibody) from the, e.g., antibody/antigen complex as is known in the art. The “Koff” also is known by the terms “dissociation rate constant” or “kd” as used interchangeably herein. This value indicates the dissociation rate of an antibody from its target antigen or separation of Ab-Ag complex over time into free antibody and antigen as shown by the equation: Ab+Ag←Ab-Ag.

The terms “equilibrium dissociation constant” or “K_{D},” as used interchangeably herein, refer to the value obtained in a titration measurement at equilibrium, or by dividing the dissociation rate constant (koff) by the association rate constant (kon). The association rate constant, the dissociation rate constant, and the equilibrium dissociation constant are used to represent the binding affinity of a binding protein, e.g., antibody, to an antigen. Methods for determining association and dissociation rate constants are well known in the art. Using fluorescence-based techniques offers high sensitivity and the ability to examine samples in physiological buffers at equilibrium. Other experimental approaches and instruments, such as a Biacore® (biomolecular interaction analysis) assay, can be used (e.g., instrument available from Biacore International AB, a GE Healthcare company, Uppsala, Sweden). Additionally, a KinExA® (Kinetic Exclusion Assay) assay, available from Sapidyne Instruments (Boise, Idaho), can also be used.

The terms “crystal” and “crystallized” as used herein, refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ed., pp. 201-16, Oxford University Press, New York, New York, (1999). The term “linker” is used to denote polypeptides comprising two or more amino acid residues joined by peptide bonds and are used to link one or more antigen binding portions. Such linker polypeptides are well known in the art (see, e.g., Holliger, P. et al., (1993) Proc. Natl. Acad. Sci. USA 90: 6444-6448; Poljak, R. J. et al., (1994) Structure 2:1121-1123). Exemplary linkers include, but are not limited to, ASTKGPSVFPLAP (SEQ ID NO: 44), ASTKGP (SEQ ID NO: 45); TVAAPSVFIFPP (SEQ ID NO: 46); TVAAP (SEQ ID NO: 47); AKTTPKLEEGEFSEAR (SEQ ID NO: 48); AKTTPKLEEGEFSEARV (SEQ ID NO: 49); AKTTPKLGG (SEQ ID NO: 50); SAKTTPKLGG (SEQ ID NO: 51); SAKTTP (SEQ ID NO: 52); RADAAP (SEQ ID NO: 53); RADAAPTVS (SEQ ID NO: 54); RADAAAAGGPGS (SEQ ID NO: 55); RADAAAA(G4S)4 (SEQ ID NO: 56); SAKTTPKLEEGEFSEARV (SEQ ID NO: 57); ADAAP (SEQ ID NO: 58); ADAAPTVSIFPP (SEQ ID NO: 59); QPKAAP (SEQ ID NO: 60); QPKAAPSVTLFPP (SEQ ID NO: 61); AKTTPP (SEQ ID NO: 62); AKTTPPSVTPLAP (SEQ ID NO: 63); AKTTAP (SEQ ID NO: 64); AKTTAPSVYPLAP (SEQ ID NO: 65); GGGGSGGGGSGGGGS (SEQ ID NO: 66); GENKVEYAPALMALS (SEQ ID NO: 67); GPAKELTPLKEAKVS (SEQ ID NO: 68); GHEAAAVMQVQYPAS (SEQ ID NO: 69); TVAAPSVFIFPPTVAAPSVFIFPP (SEQ ID NO: 70); and ASTKGPSVFPLAPASTKGPSVFPLAP (SEQ ID NO: 71).

“Label” and “detectable label” or “detectable moiety” mean a moiety attached to a specific binding partner, such as an antibody or an analyte, e.g., to render the reaction between members of a specific binding pair, such as an antibody and an analyte, detectable, and the specific binding partner, e.g., antibody or analyte, so labeled is referred to as “detectably labeled.” Thus, the term “labeled binding protein” as used herein, refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ¹⁸F. ¹¹C, ¹³N, ¹⁵O, ⁶⁸Ga, ¹⁸F, ⁸⁹Zr, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm); chromogens; fluorescent labels (e.g., FITC, rhodamine, and lanthanide phosphors); enzymatic labels (e.g., horseradish peroxidase, luciferase, and alkaline phosphatase); chemiluminescent markers; biotinyl groups; predetermined polypeptide epitopes recognized by a secondary reporter (e.g., leucine zipper pair sequences, binding sites for secondary antibodies, metal binding domains, and epitope tags); and magnetic agents, such as gadolinium chelates. Representative examples of labels commonly employed for immunoassays include moieties that produce light, e.g., acridinium compounds, and moieties that produce fluorescence, e.g., fluorescein. Other labels are described herein. In this regard, the moiety itself may not be detectably labeled but may become detectable upon reaction with yet another moiety. Use of “detectably labeled” is intended to encompass the latter type of detectable labeling.

In some embodiments, the binding affinity of an antibody, or antigen binding portion thereof, to an antigen (e.g., protein complex), such as presenting molecule-proTGFβ1 complexes, is determined using BLI (e.g., an Octet® assay). A BLI (e.g., Octet®) assay is an assay that determines one or more a kinetic parameters indicative of binding between an antibody and antigen. In some embodiments, an Octet® system (Fort6Bio®, Menlo Park, CA) is used to determine the binding affinity of an antibody, or antigen binding portion thereof, to presenting molecule-proTGFβ1 complexes. For example, binding affinities of antibodies may be determined using the ForteBio Octet® QKe dip and read label free assay system utilizing bio-layer interferometry. In some embodiments, antigens are immobilized to biosensors (e.g., streptavidin-coated biosensors) and the antibodies and complexes (e.g., biotinylated presenting molecule-proTGFβ1 complexes) are presented in solution at high concentration (50 μg/mL) to measure binding interactions. In some embodiments, the binding affinity of an antibody, or antigen binding portion thereof, to a presenting molecule-proTGFβ1 complex is determined using the protocol outlined herein.

Characterization of Exemplary Antibodies Against proTGFβ1

### Binding Profiles

Exemplary antibodies according to the present disclosure include those having enhanced binding activities (e.g., subnanomolar KD). Included are a class of high-affinity, context-independent antibodies capable of selectively inhibiting TGFβ1 activation. Note that the term “context independent” is used herein with a greater degree of stringency as compared to previous more general usage. According to the present disclosure, the term confers a level of uniformity in relative affinities (i.e., unbias) that the antibody can exert towards different antigen complexes. Thus, the context-independent antibody of the present disclosure is capable of targeting multiple types of TGFβ1 precursor complexes (e.g., presenting molecule-proTGFβ1 complexes) and of binding to each such complex with equivalent affinities (i.e., no greater than three-fold differences in relative affinities across the complexes) with K_{D} values lower than 10 nM, preferably lower than 5 nM, more preferably lower than 1 nM, even more preferably lower than 100 pM, as measured by, for example, MSD-SET. As presented below, many antibodies encompassed by the disclosure have K_{D} values in a sub-nanomolar range.

Thus, the antibodies are capable of specifically binding to each of the human presenting molecule-proTGFβ1 complexes (sometimes referred to as “Large Latency Complex” which is a ternary complex comprised of a proTGFβ1 dimer coupled to a single presenting molecule), namely, LTBP1-proTGFβ1, LTBP3-proTGFβ1, GARP-proTGFβ1 and LRRC33-proTGFβ1. Typically, recombinantly produced, purified protein complexes are used as antigens (e.g., antigen complexes) to evaluate or confirm the ability of an antibody to bind the antigen complexes in suitable in vitro binding assays. Such assays are well known in the art and include, but are not limited to Bio-Layer Interferometry (BLI)-based assays (such as Octet®) and solution equilibrium titration-based assays (such as MSD-SET).

BLI-based binding assays are widely used in the art for measuring affinities and kinetics of antibodies to antigens. It is a label-free technology in which biomolecular interactions are analyzed on the basis of optical interference. One of the proteins, for example, an antibody being tested, can be immobilized on the biosensor tip. When the other protein in solution, for example, an antigen, becomes bound to the immobilized antibody, it causes a shift in the interference pattern, which can be measured in real-time. This allows the monitoring of binding specificity, rates of association and dissociation, as well as concentration dependency. Thus, BLI is a kinetic measure that reveals the dynamics of the system. Due to its ease of use and fast results, BLI-based assays such as the Octet® system (available from Fort6Bio®/Molecular Devices®, Fremont California), are particularly convenient when used as an initial screening method to identify and separate a pool of “binders” from a pool of “non-binders” or “weak binders” in the screening process.

BLI-based binding assays revealed that the novel antibodies are characterized as “context-balanced/context-independent” antibodies when binding affinity is measured by Octet®. As can be seen in Table summarizing BLI-based binding profiles of non-limiting examples of antibodies, these antibodies show relatively uniform KD values in a sub-nanomolar range across the four target complexes, with relatively low matrix-to-cell differentials (no greater than five-fold bias) (see column (H)). This can be contrasted against the previously identified antibody Ab3, provided as a reference antibody, which shows significantly higher relative affinities towards matrix-associated complexes (27+fold bias) over cell-associated complexes.

Table 5 below provides non-limiting examples of context-independent proTGFβ1 antibodies encompassed by the present disclosure. The table provides representative results from in vitro binding assays, as measured by Octet®. Similar results are also obtained by an SPR-based technique (Biacore® System).

Column (A) of the table lists monoclonal antibodies with discrete amino acid sequences. Ab3 (shown in bold) is a reference antibody identified previously, which was shown to be potent in cell-based assays; efficacious in various animal models; and, with a clean toxicology profile (disclosed in: WO 2018/129329). Columns (B), (D), (E) and (F) provide affinities of each of the listed antibodies, measured in K_{D}. Column (B) shows the affinity to a recombinant human LTBP1-proTGFβ1 complex; column (C) shows the affinity to a recombinant human LTBP3-proTGFβ1 complex; (E) shows the affinity to a recombinant human GARP-proTGFβ1 complex; and (F) shows the affinity to a recombinant human LRRC33-proTGFβ1 complex, of each of the antibodies. Average K_{D} values of (B) and (C) are shown in the corresponding column (D), which collectively represents affinities of the antibodies to ECM- or matrix-associated proTGFβ1 complexes. Similarly, Average K_{D} values of (E) and (F) are shown in the corresponding column (G), which collectively represents affinities of the antibodies to cell-surface or cell-associated proTGFβ1 complexes. Finally, relative ratios between the average K_{D} values from columns (D) and (G) are expressed as “fold bias” in column (H). Thus, the greater the number of column (H) is, the greater bias exists for the particular antibody, when comparing binding preferences of the antibody for matrix-associated complexes and cell-surface complexes. This is one way of quantitatively representing and comparing inherent bias of antibodies to their target complexes. Such analyses may be useful in guiding the selection process for a candidate antibody for particular therapeutic use.

TABLE 5
Non-limiting examples of context-independent
TGFβ1 antibodies and KD values measured by BLI

| | Matrix-associated proTGFb1 | | | Cell-associated proTGFb1 | | | (H) |
|---|---|---|---|---|---|---|---|
| | | | (D) | | | (G) | G/D |
| (A) | (B) | (C) | ECM AVRG | (E) | (F) | Cell AVRG | (fold |
| Ab Ref | hLTBP1 | hLTBP3 | (nM) | hGARP | hLRRC33 | (nM) | bias) |
| Ab3 | 4.70E−10 | 4.59E−10 | 0.4645 | 1.73E−08 | 8.52E−09 | 12.91 | 27.79 |
| Ab21 | 2.25E−10 | 2.68E−10 | 0.2465 | 8.33E−10 | 4.55E−10 | 0.644 | 2.613 |
| Ab22 | 3.18E−10 | 3.29E−10 | 0.3235 | 9.74E−10 | 4.15E−10 | 0.6945 | 2.147 |
| Ab23 | 4.17E−10 | 4.68E−10 | 0.4425 | 1.34E−09 | 4.55E−10 | 0.8975 | 2.028 |
| Ab24 | 2.46E−10 | 1.98E−10 | 0.222 | 6.65E−10 | 4.10E−10 | 0.5375 | 2.421 |
| Ab25 | 2.17E−10 | 1.52E−10 | 0.1845 | 4.88E−10 | 4.09E−10 | 0.4485 | 2.431 |
| Ab26 | 2.21E−10 | 1.73E−10 | 0.197 | 6.25E−10 | 3.60E−10 | 0.4925 | 2.500 |
| Ab27 | 1.78E−10 | 2.38E−10 | 0.208 | 4.24E−10 | 2.99E−10 | 0.3615 | 1.738 |
| Ab28 | 3.40E−10 | 3.16E−10 | 0.328 | 7.97E−10 | 4.09E−10 | 0.603 | 1.838 |
| Ab29 | 1.89E−10 | 1.21E−10 | 0.155 | 3.07E−10 | 3.02E−10 | 0.3045 | 1.965 |
| AB30 | 3.32E−10 | 2.61E−10 | 0.2965 | 8.33E−10 | 5.35E−10 | 0.684 | 2.307 |
| Ab31 | 2.36E−10 | 1.81E−10 | 0.2085 | 5.81E−10 | 4.10E−10 | 0.4955 | 2.376 |
| Ab6 | 2.07E−10 | 1.23E−10 | 0.165 | 4.04E−10 | 3.36E−10 | 0.37 | 2.242 |
| Ab32 | 2.69E−10 | 2.15E−10 | 0.242 | 4.96E−10 | 6.98E−10 | 0.597 | 2.467 |
| Ab33 | 1.79E−10 | 1.11E−10 | 0.145 | 2.65E−10 | 3.39E−10 | 0.302 | 2.083 |

The disclosure provides a class of high-affinity, context-independent antibodies, each of which is capable of binding with equivalent affinities to each of the four known presenting molecule-proTGFβ1 complexes, namely, LTBP1-proTGFβ1, LTBP3-proTGFβ1, GARP-proTGFβ1, and LRRC33-proTGFβ1. In some embodiments, the antibody binds each of the presenting molecule-proTGFβ1 complexes with equivalent or higher affinities, as compared to the previously described reference antibody, Ab3. According to the disclosure, such antibody specifically binds each of the aforementioned complexes with an affinity (determined by K_{D}) of ≤5 nM as measured by a suitable in vitro binding assay, such as Biolayer Interferometry and surface plasmon resonance. In some embodiments, the antibody or the fragment binds a human LTBP1-proTGFβ1 complex with an affinity of ≤5 nM, ≤4 nM, ≤3 nM, ≤2 nM, ≤1 nM, ≤5 nM or ≤0.5 nM. In some embodiments, the antibody or the fragment binds a human LTBP3-proTGFβ1 complex with an affinity of ≤5 nM, ≤4 nM, ≤3 nM, ≤2 nM, ≤1 nM, ≤5 nM or ≤0.5 nM. In some embodiments, the antibody or the fragment binds a human GARP-proTGFβ1 complex with an affinity of ≤5 nM, ≤4 nM, ≤3 nM, ≤2 nM, ≤1 nM, ≤5 nM or ≤0.5 nM. In some embodiments, the antibody or the fragment binds a human LRRC33-proTGFβ1 complex with an affinity of ≤5 nM, ≤4 nM, ≤3 nM, ≤2 nM, ≤1 nM or ≤0.5 nM.

In certain embodiments, such antibody is human- and murine-cross-reactive. Thus, in some embodiments, the antibody or the fragment binds a murine LTBP1-proTGFβ1 complex with an affinity of ≤5 nM, ≤4 nM, ≤3 nM, ≤2 nM, ≤1 nM, ≤5 nM or ≤0.5 nM. In some embodiments, the antibody or the fragment binds a murine LTBP3-proTGFβ1 complex with an affinity of ≤5 nM, ≤4 nM, ≤3 nM, ≤2 nM, ≤1 nM or ≤0.5 nM. In some embodiments, the antibody or the fragment binds a murine GARP-proTGFβ1 complex with an affinity of ≤5 nM, ≤4 nM, ≤3 nM, ≤2 nM, ≤1 nM or ≤0.5 nM. In some embodiments, the antibody or the fragment binds a murine LRRC33-proTGFβ1 complex with an affinity of ≤5 nM, ≤4 nM, ≤3 nM, ≤2 nM, ≤1 nM or ≤0.5 nM.

As shown, the proTGFβ1 antibodies of the present disclosure have particularly high affinities for matrix-associated proTGFβ1 complexes. In some embodiments, the average K_{D} value of the matrix-associated complexes (i.e., LTBP1-proTGFβ1 and LTBP3-proTGFβ1) is ≤1 nM or ≤0.5 nM.

As shown, the proTGFβ1 antibodies of the present disclosure have high affinities for cell-associated proTGFβ1 complexes. In some embodiments, the average K_{D} value of the cell-associated complexes (i.e., GARP-proTGFβ1 and LRRC33-proTGFβ1) is ≤2 nM or ≤1 nM.

The high-affinity proTGFβ1 antibodies of the present disclosure are characterized by their uniform (unbiased) affinities towards the all four antigen complexes (compare, for example, to Ab3). No single antigen complex among the four known presenting molecule-proTGFβ1 complexes described herein deviates significantly in K_{D}. In other words, more uniform binding activities have been achieved by the present disclosure relative to previously described proTGFβ1 antibodies (including Ab3) in that each such antibody shows equivalent affinities across the four antigen complexes. In some embodiments, the antibody or the fragment shows unbiased or uniform binding profiles, characterized in that the difference (or range) of affinities of the antibody or the fragments across the four proTGFβ1 antigen complexes is no more than five-fold between the lowest and the highest K_{D} values. In some embodiments, the relative difference (or range) of affinities is no more than three-fold.

The concept of “uniformity” or lack of bias is further illustrated in Table 5. Average K_{D} values between the two matrix-associated and cell-associated complexes are calculated, respectively (see columns (D) and (G)). These average K_{D} values can then be used to ask whether bias in binding activities exists between complexes associated with matrix vs. complexes associated with cell surface (e.g., immune cells). Bias may be expressed as “fold-difference” in the average K_{D} values, as illustrated in Table 5. As compared to the previously described antibody, Ab3, the high-affinity, context-independent proTGFβ1 antibodies encompassed by the present disclosure are remarkably unbiased in that many show no more than three-fold difference in average K_{D} values between matrix- and cell-associated complexes (compare this to 25+fold bias in Ab3).

Accordingly, a class of context-independent monoclonal antibodies or fragments is provided, each of which is capable of binding with equivalent affinities to each of the following presenting molecule-proTGFβ1 complexes with an affinity of: 1 nM as measured by Biolayer Interferometry or surface plasmon resonance: LTBP1-proTGFβ1, LTBP3-proTGFβ1, GARP-proTGFβ1, and LRRC33-proTGFβ1. Such antibody specifically binds each of the aforementioned complexes with an affinity of ≤5 nM as measured by Biolayer Interferometry or surface plasmon resonance, wherein the monoclonal antibody or the fragment shows no more than a three-fold bias in affinity towards any one of the above complexes relative to the other complexes, and wherein the monoclonal antibody or the fragment inhibits release of mature TGFβ1 growth factor from each of the proTGFβ1 complexes but not from proTGFβ2 or proTGFβ3 complexes.

Whilst the kinetics of binding profiles (e.g., “on” and “off” rates) obtainable from BLI-based assays provide useful information, Applicant of the present disclosure contemplated that, based on the mechanism of action of the activation inhibitors disclosed herein, that is, antibodies that work by binding to a tethered (e.g., tissue-localized) inactive (e.g., latent) target thereby preventing it from getting activated, binding properties measured at equilibrium might more accurately reflect their in vivo behavior and potency. To put this in perspective, as an example, antibodies with fast “on” rate (“Kₒₙ”) which would be reflected in binding measurements obtained by BLI, may provide relevant parameters for evaluating neutralizing antibodies (e.g., antibodies that directly target and must rapidly sequester the active, soluble growth factor itself for them to function as effective inhibitors). However, the same may not necessarily apply for antibodies that function as activation inhibitors, such as those disclosed herein. As described, the mechanism of action of the novel TGFβ1 inhibitors of the present disclosure is via the inhibition of the activation step, which is achieved by targeting the tissue/cell-tethered latent complex, as opposed to sequestration of soluble, post-activation growth factor. This is because an activation inhibitor of TGFβ1 targets the inactive precursor localized to respective tissues (e.g., within the ECM, immune cell surface, etc.) thereby preemptively prevent the mature growth factor from being released from the complex. This mechanism of action is thought to allow the inhibitor to achieve target saturation (e.g., equilibrium) in vivo, without the need for rapidly competing for transient growth factor molecules against endogenous receptors as required by conventional neutralizing inhibitors.

Taking this difference in the mechanism of action into consideration, further evaluation of binding properties was carried out by the use of another mode of in vitro binding assays that allows the determination of affinity at equilibrium.

In view of this, it is contemplated that assays that measure binding affinities of such antibodies at equilibrium may more accurately represent the mode of target engagement in vivo. Thus, MSD-SET-based binding assays (or other suitable assays) may be performed, as exemplified in Table 6 below.

Solution equilibrium titration (“SET”) is an assay whereby binding between two molecules (such as an antigen and an antibody that binds the antigen) can be measured at equilibrium in a solution. For example, Meso-Scale Discovery (“MSD”)-based SET, or MSD-SET, is a useful mode of determining dissociation constants for particularly high-affinity protein-protein interactions at equilibrium (see, for example: Ducata et al., (2015) J Biomolecular Screening 20(10): 1256-1267). The SET-based assays are particularly useful for determining KD values of antibodies with sub-nanomolar (e.g., picomolar) affinities.

TABLE 6
Non-limiting examples of high-affinity context-independent TGFβ1 antibodies
(hIgG4) and KD values measured by MSD-SET (“h” denotes human complex)

| | Matrix-associated proTGFβ1 | | | Cell-associated proTGFβ1 | | | (H) |
|---|---|---|---|---|---|---|---|
| | | | (D) | | | (G) | G/D |
| (A) | (B) | (C) | ECM AVRG | (E) | (F) | Cell AVRG | (fold |
| Ab Ref | hLTBP1 | hLTBP3 | (nM) | hGARP | hLRRC33 | (nM) | bias) |
| C1 | 3.30E−08 | 1.40E−08 | 23.2 | 5.10E−09 | 2.20E−09 | 3.65 | 0.16 |
| C2 | 2.10E−08 | 1.20E−08 | 16.5 | 8.80E−09 | 6.10E−09 | 7.45 | 0.48 |
| Ab3 | 1.30E−08 | 1.62E−08 | 14.6 | 2.80E−08 | 3.50E−08 | 31.5 | 2.16 |
| Ab6 | 1.8E−11 | 2.9E−11 | 0.024 | 2.7E−11 | 6.3E−11 | 0.045 | 1.88 |
| Ab22 | 5.00E−11 | 3.30E−11 | 0.042 | 2.70E−11 | 2.00E−10 | 0.114 | 2.71 |
| Ab24 | 2.40E−11 | 2.10E−11 | 0.023 | 1.90E−11 | 1.80E−10 | 0.100 | 4.35 |
| Ab26 | 2.80E−11 | 2.30E−11 | 0.026 | 1.40E−11 | 1.30E−10 | 0.072 | 2.77 |
| Ab29 | 1.20E−11 | 1.10E−11 | 0.012 | 5.50E−12 | 4.30E−11 | 0.024 | 2.00 |
| Ab30 | 3.10E−11 | 2.60E−11 | 0.029 | 2.20E−11 | 1.40E−10 | 0.081 | 2.80 |
| Ab31 | 1.90E−11 | 1.40E−11 | 0.017 | 1.90E−11 | 9.60E−11 | 0.058 | 3.41 |
| Ab32 | 3.70E−11 | 2.60E−11 | 0.032 | 1.50E−11 | 8.70E−11 | 0.051 | 1.60 |
| Ab33 | 1.10E−11 | 7.00E−12 | 0.009 | 7.80E−12 | 4.60E−11 | 0.027 | 3.00 |
| Ab4 | 4.6E−9 | 5.5E−9 | 5.05 | 2.5E−9 | 2.1E−9 | 2.3 | 0.42 |

Table 6 also includes three previously described TGFβ1-selective antibodies (C1, C2 and Ab3) as reference antibodies. C1 and C2 were first disclosed in PCT/US2017/021972 published as WO 2017/156500 (corresponding to “Ab1” and “Ab2” therein), and Ab3 was described in PCT/US2018/012601 published as WO 2018/129329 (corresponding to “Ab3” therein).

As can be seen from the affinity data provide in Table 6, binding activities of the novel antibodies according to the present disclosure are significantly higher than the previously identified reference antibodies. Moreover, the novel TGFβ1 antibodies are “context-independent” in that they bind to each of the human LLC complexes with equivalent affinities (e.g., ˜sub-nanomolar range, e.g., with K_{D} of ≤1 nM). The high-affinity, context-independent binding profiles suggest that these antibodies may be advantageous for use in the treatment of TGFβ1-related indications that involve dysregulation of both the ECM-related and immune components, such as cancer.

For solution equilibrium titration-based binding assays, protein complexes that comprise one of the presenting molecules such as those shown above may be employed as antigen (presenting molecule-TGFβ1 complex, or an LLC). Test antibodies are allowed to form antigen-antibody complex in solution. Antigen-antibody reaction mixtures are incubated to allow an equilibrium to be reached; the amount of the antigen-antibody complex present in the assay reactions can be measured by suitable means well known in the art. As compared to BLI-based assays, SET-based assays are less affected by on/off rates of the antigen-antibody complex, allowing sensitive detection of very high affinity interactions. As shown in Table 6, in the present disclosure, certain high-affinity inhibitors of TGFβ1 show a sub-nanomolar (e.g., picomolar) range of affinities across all large latent complexes tested, as determined by SET-based assays.

Accordingly, a class of context-independent monoclonal antibodies or fragments is provided, each of which is capable of binding with equivalent affinities to each of the following human presenting molecule-proTGFβ1 complexes with a K_{D} of <1 nM as measured by a solution equilibrium titration assay, such as MSD-SET: hLTBP1-proTGFβ1, hLTBP3-proTGFβ1, hGARP-proTGFβ1, and hLRRC33-proTGFβ1. Such antibody specifically binds each of the aforementioned complexes with a K_{D} of ≤1 nM as measured by MSD-SET, and wherein the monoclonal antibody or the fragment inhibits release of mature TGFβ1 growth factor from each of the proTGFβ1 complexes but not from proTGFβ2 or proTGFβ3 complexes. In certain embodiments, such antibody or the fragment binds each of the aforementioned complexes with a K_{D} of 500 pM or less (i.e., ≤500 pM), 250 pM or less (i.e., ≤250 pM), or 200 pM or less (i.e., ≤200 pM). Even more preferably, such antibody or the fragment binds each of the aforementioned complexes with a K_{D} of 100 pM or less (i.e., ≤100 pM). In some embodiments, the antibody or the fragment does not bind to free TGFβ1 growth factor which is not associated with the prodomain complex. In some embodiments, the antibody or the fragment does not bind to LTBP1/TGFβ2 or LTBP3/TGFβ3 LLCs. This can be tested or confirmed by suitable in vitro binding assays known in the art, such as biolayer interferometry.

In further embodiments, such antibodies or the fragments are also cross-reactive with murine (e.g., rat and/or mouse) and/or non-human primate (e.g., cyno) counterparts. To give but one example, Ab6 is capable of binding with high affinity to each of the large latent complexes of multiple species, including: human, murine, rat, and cynomolgus monkey, as exemplified in Table 7 below.

TABLE 7
Non-limiting example of a TGFβ1 antibody with cross-species reactivities
as measured by MSD-SET (“h” denotes human; “m” denotes murine)

| Ag | hLTBP1- | hLTBP3- | hGARP- | hLRRC33- | mLTBP1- | mLTBP3- | mGARP- | mLRRC33- |
|---|---|---|---|---|---|---|---|---|
| complex | proTGFβ1 | proTGFβ1 | proTGFβ1 | proTGFβ1 | proTGFβ1 | proTGFβ1 | proTGFβ1 | proTGFβ1 |
| Ab6 | 1.80E−11 | 2.90E−11 | 2.70E−11 | 6.30E−11 | 2.40E−11 | 2.80E−11 | 2.10E−11 | 4.80E−11 |

Surface plasmon resonance (SPR) provides useful binding kinetics information with good resolution and sensitivity, which enables detection of unlabeled biomolecular interactants (such as antibody-antigen interactions) in real time. The SPR-based biosensors (such as Biacore systems) can be used in determination of active concentration as well as characterization of molecular interactions in terms of both affinity and chemical kinetics. With respect to antibodies that target latent prodomain complex and inhibit the activation step of growth factor from the latent complex, in addition to having high overall affinities (typically expressed as the equilibrium dissociation constant or K_{D}), it may be particularly advantageous to have slow off rates, or K_{OFF}, As previously exemplified in Example 1 of PCT/US2019/041373, Ab6, which is an activation inhibitor of TGFβ1, binds each LLC with a K_{D} of less than 0.5 nM with K_{OFF} of less than 10.0E-4 (1/s), as measured by SPR. The off rate of an antibody may therefore be an important binding kinetics criterion for selection consideration for a therapeutic antibody to be manufactured and for use in human therapy described herein.

Accordingly, the invention includes a TGFβ inhibitor which is an antibody or antigen-binding fragment thereof, for use in the treatment of cancer in a subject (according to the present disclosure), wherein the antibody or the fragment with a K_{OFF} of less than 10.0E-4 (1/s) is selected, wherein optionally the selected antibody has a K_{D} of less than 0.5 nM as measured by SPR.

The invention further includes a method for manufacturing a pharmaceutical composition comprising a TGFβ inhibitor which is an antibody or antigen-binding fragment thereof, for use in the treatment of cancer in a subject (according to the present disclosure), the method comprising the step of selecting an antibody or antigen-binding fragment which has a K_{OFF} of less than 10.0E-4 (1/s) and optionally has a K_{D} of less than 0.5 nM as measured by SPR.

### Potency

Antibodies disclosed herein may be broadly characterized as “functional antibodies” for their ability to inhibit TGFβ1 signaling. As used herein, “a functional antibody” confers one or more biological activities by virtue of its ability to bind a target protein (e.g., antigen), in such a way as to modulate its function. Functional antibodies therefore broadly include those capable of modulating the activity/function of target molecules (i.e., antigen). Such modulating antibodies include inhibiting antibodies (or inhibitory antibodies) and activating antibodies. The present disclosure is drawn to antibodies which can inhibit a biological process mediated by TGFβ signaling associated with multiple contexts of TGFβ1. Inhibitory agents used to carry out the present disclosure, such as the antibodies described herein, are intended to be TGFβ1-selective and not to target or interfere with TGFβ2 and TGFβ3 when administered at a therapeutically effective dose (dose at which sufficient efficacy is achieved within acceptable toxicity levels). The novel antibodies of the present disclosure have enhanced inhibitory activities (potency) as compared to previously identified activation inhibitors of TGFβ1.

In some embodiments, potency of an inhibitory antibody may be measured in suitable cell-based assays, such as CAGA reporter cell assays described herein. Generally, cultured cells, such as heterologous cells and primary cells, may be used for carrying out cell-based potency assays. Cells that express endogenous TGFβ1 and/or a presenting molecule of interest, such as LTBP1, LTBP3, GARP and LRRC33, may be used. Alternatively, exogenous nucleic acids encoding protein(s) of interest, such as TGFβ1 and/or a presenting molecule of interest, such as LTBP1, LTBP3, GARP and LRRC33, may be introduced into such cells for expression, for example by transfection (e.g., stable transfection or transient transfection) or by viral vector-based infection. In some embodiments, LN229 cells are employed for such assays. The cells expressing TGFβ1 and a presenting molecule of interest (e.g., LTBP1, LTBP3, GARP or LRRC33) are grown in culture, which “present” the large latent complex either on cell surface (when associated with GARP or LRRC33) or deposit into the ECM (when associated with an LTBP). Activation of TGFβ1 may be triggered by integrin, expressed on another cell surface. The integrin-expressing cells may be the same cells co-expressing the large latent complex or a separate cell type. Reporter cells are added to the assay system, which incorporates a TGFβ-responsive element. In this way, the degree of TGFβ activation may be measured by detecting the signal from the reporter cells (e.g., TGFβ-responsive reporter genes, such as luciferase coupled to a TGFβ-responsive promoter element) upon TGFβ activation. Using such cell-based assay systems, inhibitory activities of the antibodies can be determined by measuring the change (reduction) or difference in the reporter signal (e.g., luciferase activities as measured by fluorescence readouts) either in the presence or absence of test antibodies. Such assays are exemplified in PCT/US2019/041373 at Example 2.

Thus, in some embodiments, the inhibitory potency (IC₅₀) of the novel antibodies of the present disclosure calculated based on cell-based reporter assays for measuring TGFβ1 activation (such as LN229 cell assays described elsewhere herein) may be 5 nM or less, measured against each of the hLTBP1-proTGFβ1, hLTBP3-proTGFβ1, hGARP-proTGFβ1 and hLRRC33-proTGFβ1 complexes. In some embodiments, the antibodies have an IC₅₀ of 2 nM or less (i.e., ≤2 nM) measured against each of the LLCs. In certain embodiments, the IC₅₀ of the antibody measured against each of the LLC complexes is 1 nM or less. In some embodiments, the antibody has an IC₅₀ of less than 1 nM against each of the hLTBP1-proTGFβ1, hLTBP3-proTGFβ1, hGARP-proTGFβ1 and hLRRC33-proTGFβ1 complexes.

TABLE 8
Inhibitory potencies (in IC50) of select antibodies
as measured by reporter cell assays

IC₅₀ (nM)

| Ab | hLTBP1- | hLTBP3- | hGARP- | hLRRC33- |
|---|---|---|---|---|
| Ref. | proTGFβ1 | proTGFβ1 | proTGFβ1 | proTGFβ1 |
| Ab4 | 5.2 | 5.6 | 0.8 | 3.5 |
| Ab5 | 1.3 | 1.0 | 0.1 | 0.6 |
| Ab6 | 1.0-2.7 | 0.8-2.7 | 0.3-1.6 | 0.5-1.9 |
| Ab21 | 1.6 | 0.8 | 0.4 | 0.6 |
| Ab23 | 0.8 | 0.9 | 0.3 | 0.6 |
| Ab25 | 6.1 | 0.51 | 0.4 | 0.7 |
| Ab26 | 0.7 | 0.7 | 0.3 | 0.3 |
| Ab29 | 0.5 | 0.8 | 0.3 | 0.5 |
| Ab33 | 1.6 | 1.1 | 0.2 | 0.7 |

Activation of TGFβ1 may be triggered by an integrin-dependent mechanism or protease-dependent mechanism. The inhibitory activities (e.g., potency) of the antibodies according to the present disclosure may be evaluated for the ability to block TGFβ1 activation induced by one or both of the modes of activation. The reporter cell assays described above are designed to measure the ability of the antibodies to block or inhibit integrin-dependent activation of TGFβ1 activation. Inhibitory potency may also be assessed by measuring the ability of the antibodies to block protease-induced activation of TGFβ1. Non-limiting embodiments of such assays were previously disclosed. See, e.g., PCT/US2019/041373 at Example 3. Accordingly, in some embodiments of the disclosure, the isoform-selective inhibitor according to the present disclosure is capable of inhibiting integrin-dependent activation of TGFβ1 and protease-dependent activation of TGFβ1. Such inhibitor may be used to treat a TGFβ31-related indication characterized by EDM dysregulation involving protease activities. For example, such TGFβ31-related indication may be associated with elevated myofibroblasts, increased stiffness of the ECM, excess or abnormal collagen deposition, or any combination thereof. Such conditions include, for example, fibrotic disorders and cancer comprising a solid tumor (such as metastatic carcinoma) or myelofibrosis.

In some embodiments, potency may be evaluated in suitable in vivo models as a measure of efficacy and/or pharmacodynamics effects. For example, if the first antibody is efficacious in an in vivo model at a certain concentration, and the second antibody is equally efficacious at a lower concentration than the first in the same in vivo model, then, the second antibody can be said to me more potent than the first antibody. Any suitable disease models known in the art may be used to assess relative potencies of TGFβ1 inhibitors, depending on the particular indication of interest, e.g., cancer models and fibrosis models. Preferably, multiple doses or concentrations of each test antibody are included in such studies.

Similarly, pharmacodynamics (PD) effects may be measured to determine relative potencies of inhibitory antibodies. Commonly used PD measures for the TGFβ signaling pathway include, without limitation, phosphorylation of SMAD2/3 and expression of downstream effector genes, the transcription of which is sensitive to TGFβ activation, such as those with a TGFβ-responsive promoter element (e.g., Smad-binding elements). In some embodiments, the antibodies of the present disclosure are capable of completely blocking disease-induced SMAD2/3 phosphorylation in preclinical fibrosis models when the animals are administered at a dose of 3 mg/kg or less. In some embodiments, the antibodies of the present disclosure are capable of reducing and/or completely blocking disease-induced SMAD2/3 phosphorylation. In some embodiments, the antibodies of the present disclosure are capable of reducing and/or completely blocking disease-induced SMAD2 phosphorylation (e.g., regardless of any change in SMAD3), e.g., in the nucleus. In some embodiments, reduction is measured as a ratio of phosphorylated SMAD2/3 over total SMAD2/3. In some embodiments, reduction is measured as a ratio of phosphorylated SMAD2 over total SMAD2. In some embodiments, the antibodies of the present disclosure are capable of reducing nuclear localization of phosphorylated SMAD2, as measured, for example, by IHC.

In some embodiments, measurement of nuclear localization of phosphorylated Smad2 (P-Smad2) can use one or more digital image analysis parameters to identify degrees of P-Smad2 nucleus staining intensities (e.g., a digital image analysis parameter that allows visualization and measurement of IHC signal intensity of an individual nucleus, e.g., a nucleus mask). P-Smad2 positive nuclei can be sorted into categories (e.g., 1+, 2+, 3+) based on chromogenic intensity relative to normalized scanning parameters. The analysis can allow for data to be considered in multiple contexts (e.g., % Nuclear Positivity, H-Score, Density), and can be combined with additional analysis parameters (e.g., compartment area, total number of quantified cells, number of P-Smad2 positive cells, and/or number or percent of 0 P-Smad2, 1+P-Smad2, 2+P-Smad2, or 3+P-Smad2 cells). Pseudocolor image masks can be applied to each staining category for visualization and QC purposes. Exemplary tools for analyzing nuclear masking include, but are not limited to, software developed by Visiopharm, Indica Labs (e.g., HALO® imaging analysis platform), and Flagship Biosciences. Without being bound by theory, in some embodiments, measuring SMAD2 phosphorylation e.g., nuclear SMAD2 phosphorylation (without measuring SMAD3) may improve the accurate detection of a treatment-related effect (e.g., therapeutic efficacy and/or target engagement). Denis et al., Development 143: 3481-90 (2016); Liu et al., J. Biol. Chem. 278: 11721-8 (2003); David et al., Oncoimmunology 6: e1349589 (2017).

In some embodiments, P-Smad2 nuclear translocation may be used in a method of determining therapeutic efficacy in a subject administered one or more doses of a TGFβ inhibitor (e.g., Ab6). In some embodiments, a level of P-Smad2 nuclear translocation is determined in a tumor sample obtained from the subject before and after administering one or more doses of a TGFβ inhibitor (e.g., Ab6), wherein a decrease in P-Smad2 nuclear translocation after the administration as compared to before the administration indicates therapeutic efficacy. In some embodiments, the P-Smad2 nuclear translocation may be determined using immunohistochemistry. In some embodiments, the P-Smad2 nuclear translocation may be determined using nuclear masking. In some embodiments, the P-Smad2 nuclear translocation may be determined using a digital image analysis tool, such as one developed by Flagship Biosciences, Visiopharm, or Indica Labs. In some embodiments, a decrease of at least 1.1-fold, 1.2-fold, 1.3-fold, 1.4-fold, 1.5-fold, 1.6-fold, 1.7-fold, 1.8-fold, 1.9-fold, 2-fold, 2.2-fold, 2.4-fold, 2.6-fold, 2.8-fold, 3-fold, 3.5-fold, 4-fold, 4.5-fold, 5-fold, or more, in P-Smad2 nuclear translocation levels after the administration as compared to before the administration indicates therapeutic efficacy. In some embodiments, one or more additional doses of the TGFβ inhibitor (e.g., Ab6) is administered if a decrease is detected.

In some embodiments, P-Smad2 nuclear translocation may be used in a method of determining target engagement in a subject administered one or more doses of a TGFβ inhibitor (e.g., Ab6). In some embodiments, a level of P-Smad2 nuclear translocation is determined in a tumor sample obtained from the subject before and after administering one or more doses of a TGFβ inhibitor (e.g., Ab6), wherein a decrease in P-Smad2 nuclear translocation after the administration as compared to before the administration indicates target engagement. In some embodiments, the P-Smad2 nuclear translocation may be determined using immunohistochemistry. In some embodiments, the P-Smad2 nuclear translocation may be determined using nuclear masking. In some embodiments, the P-Smad2 nuclear translocation may be determined using a digital image analysis tool, such as one developed by Flagship Biosciences, Visiopharm, or Indica Labs. In some embodiments, a decrease of at least 1.1-fold, 1.2-fold, 1.3-fold, 1.4-fold, 1.5-fold, 1.6-fold, 1.7-fold, 1.8-fold, 1.9-fold, 2-fold, 2.2-fold, 2.4-fold, 2.6-fold, 2.8-fold, 3-fold, 3.5-fold, 4-fold, 4.5-fold, 5-fold, or more, in P-Smad2 nuclear translocation levels after the administration as compared to before the administration indicates target engagement. In some embodiments, one or more additional doses of the TGFβ inhibitor (e.g., Ab6) is administered if a decrease is detected.

In some embodiments, P-Smad2 nuclear translocation may be used in conjunction with other biomarkers, such as tumor CD8+ cells (e.g., percent CD8+ cells in various tumor compartments, e.g., tumor nests, stroma, margin), to determine therapeutic efficacy and/or target engagement in a subject administered one or more doses of a TGFβ inhibitor (e.g., Ab6). In some embodiments, therapeutic efficacy and/or target engagement may be indicated by, inter alia, a decrease in P-Smad2 nuclear translocation in a tumor sample obtained from the subject after the administration of a TGFβ inhibitor (e.g., Ab6) as compared to before the administration. In some embodiments, therapeutic efficacy and/or target engagement may be indicated by a decrease in P-Smad2 nuclear translocation and an increase in CD8+ cells in a tumor sample obtained from the subject after the administration as compared to before the administration. In some embodiments, therapeutic efficacy and/or target engagement may be indicated by a decrease in P-Smad2 nuclear translocation and an increase in total tumor area comprising immune-inflamed tumor nests in a tumor sample obtained from the subject after the administration as compared to before the administration. In some embodiments, more than one additional marker is used to assess therapeutic efficacy and/or target engagement in conjunction with P-Smad2 nuclear translocation. In some embodiments, the TGFβ inhibitor (e.g., Ab6) is administered in conjunction with a checkpoint inhibitor therapy. In some embodiments, additional doses of the TGFβ inhibitor (e.g., Ab6) may be administered to patients whose tumors show therapeutic efficacy and/or target engagement.

In some embodiments, the antibodies of the present disclosure are capable of significantly suppressing fibrosis-induced expression of a panel of marker genes including Acta2, Collal, Col3a1, Fn1, Itga11, Lox, Lox12, when the animals are administered at a dose of 10 mg/kg or less in the UUO model of kidney fibrosis.

In some embodiments, the selection process of an antibody or antigen-binding fragment thereof for therapeutic use may therefore include identifying an antibody or fragment that shows sufficient inhibitory potency. For example, the selection process may include a step of carrying out a cell-based TGFβ1 activation assay to measure potency (e.g., ICso) of one or more test antibodies or fragments thereof, and, selecting a candidate antibody or fragment thereof that shows desirable potency. In some embodiments, ICso for each of the human LLCs 5 nM or less. The selected antibody or the fragment may then be used in the treatment of a TGFβ1-related indication described herein.

### Binding Regions

In the context of the present disclosure, “binding region(s)” of an antigen provides a structural basis for the antibody-antigen interaction. As used herein, a “binding region” refers to the areas of interface between the antibody and the antigen, such that, when bound to the proTGFβ1 complex (“antigen”) in a physiological solution, the antibody or the fragment protects the binding region from solvent exposure, as determined by suitable techniques, such as hydrogen-deuterium exchange mass spectrometry (HDX-MS). Identification of binding regions is useful in gaining insight into the antigen-antibody interaction and the mechanism of action for the particular antibody. Identification of additional antibodies with similar or overlapping binding regions may be facilitated by cross-blocking experiments that enable epitope binning. Optionally, X-ray crystallography may be employed to identify the exact amino acid residues of the epitope that mediate antigen-antibody interactions.

The art is familiar with HDX-MS, which is a widely used technique for exploring protein conformation or protein-protein interactions in solution. This method relies on the exchange of hydrogens in the protein backbone amide with deuterium present in the solution. By measuring hydrogen-deuterium exchange rates, one can obtain information on protein dynamics and conformation (reviewed in: Wei et al., (2014) “Hydrogen/deuterium exchange mass spectrometry for probing higher order structure of protein therapeutics: methodology and applications.” Drug Discov Today. 19(1): 95-102; incorporated by reference). The application of this technique is based on the premise that when an antibody-antigen complex forms, the interface between the binding partners may occlude solvent, thereby reducing or preventing the exchange rate due to steric exclusion of solvent.

The present disclosure includes antibodies or antigen-binding fragments thereof that bind a human LLC at a region (“binding region”) comprising Latency Lasso or a portion thereof. Latency Lasso is a protein module within the prodomain. It is contemplated that many potent activation inhibitors may bind this region of a proTGFβ1 complex in such a way that the antibody binding would “lock in” the growth factor thereby preventing its release. Interestingly, this is the section of the complex where the butterfly-like elongated regions of the growth factor (e.g., corresponding to, for example, Finger-1 and Finger-2) closely interact with the cage-like structure of the prodomain. Based on the data previously disclosed in PCT/US2019/041373, it is envisaged that an antibody that tightly wraps around the binding regions identified may effectively prevent the proTGFβ1 complex from disengaging (i.e., releasing the growth factor), thereby blocking activation.

Using the HDX-MS technique, binding regions of proTGFβ1 can be determined. In some embodiments, a portion on proTGFβ1 identified to be important in binding an antibody or fragment includes at least a portion of the prodomain and at least a portion of the growth factor domain. Antibodies or fragments that bind a first binding region (“Region 1” in FIG. 41) comprising at least a portion of Latency Lasso are preferable. More preferably, such antibodies or fragments further bind a second binding region (“Region 2” in FIG. 41) comprising at least a portion of the growth factor domain at Finger-1 of the growth factor domain. Such antibodies or fragments may further bind a third binding region (“Region 3” in FIG. 41) comprising at least a portion of Finger-2 of the growth factor domain.

Additional regions within the proTGFβ1 may also contribute, directly or indirectly, to the high-affinity interaction of these antibodies disclosed herein. Regions that are considered important for mediating the high-affinity binding of the antibody to the proTGFβ1 complex may include, but are not limited to: LVKRKRIEA (SEQ ID NO: 132); LASPPSQGEVP (SEQ ID NO: 133); PGPLPEAV (SEQ ID NO: 134); LALYNSTR (SEQ ID NO: 135); REAVPEPVL (SEQ ID NO: 136); YQKYSNNSWR (SEQ ID NO: 137); RKDLGWKWIHEPKGYHANF (SEQ ID NO: 138); LGPCPYIWS (SEQ ID NO: 139); ALEPLPIV (SEQ ID NO: 140); and, VGRKPKVEQL (SEQ ID NO: 141) (based on the native sequence of human proTGFβ1).

Among regions that may contribute to the antibody-antigen interaction, in some embodiments, the high-affinity antibody of the present disclosure may bind an epitope that comprises at least one residue of the amino acid sequence KLRLASPPSQGEVPPGPLPEAVL (“Region 1”) (SEQ ID NO: 142).

In some embodiments, the high-affinity antibody of the present disclosure may bind an epitope that comprises at least one residue of the amino acid sequence RKDLGWKWIHEPKGYHANF (“Region 2”) (SEQ ID NO: 138).

In some embodiments, the high-affinity antibody of the present disclosure may bind an epitope that comprises at least one residue of the amino acid sequence VGRKPKVEQL (“Region 3”) (SEQ ID NO: 141).

In some embodiments, the high-affinity antibody of the present disclosure may bind an epitope that comprises at least one residue of the amino acid sequence KLRLASPPSQGEVPPGPLPEAVL (“Region 1”) (SEQ ID NO: 142) and at least one residue of the amino acid sequence RKDLGWKWIHEPKGYHANF (“Region 2”) (SEQ ID NO: 138).

In some embodiments, the high-affinity antibody of the present disclosure may bind an epitope that comprises at least one residue of the amino acid sequence KLRLASPPSQGEVPPGPLPEAVL (“Region 1”) (SEQ ID NO: 142) and at least one residue of the amino acid sequence VGRKPKVEQL (“Region 3”) (SEQ ID NO: 141).

In some embodiments, the high-affinity antibody of the present disclosure may bind an epitope that comprises at least one residue of the amino acid sequence KLRLASPPSQGEVPPGPLPEAVL (“Region 1”) (SEQ ID NO: 142), at least one residue of the amino acid sequence RKDLGWKWIHEPKGYHANF (“Region 2”) (SEQ ID NO: 138), and, at least one residue of the amino acid sequence VGRKPKVEQL (“Region 3”) (SEQ ID NO: 141).

In addition to contributions from Regions 1, 2 and/or 3, such epitope may further include at least one amino acid residues from a sequence selected from the group consisting of: LVKRKRIEA (SEQ ID NO: 132); LASPPSQGEVP (SEQ ID NO: 133); PGPLPEAV (SEQ ID NO: 134); LALYNSTR (SEQ ID NO: 135); REAVPEPVL (SEQ ID NO: 136); YQKYSNNSWR (SEQ ID NO: 137); RKDLGWKWIHEPKGYHANF (SEQ ID NO: 138); LGPCPYIWS (SEQ ID NO: 139); ALEPLPIV (SEQ ID NO: 140); and, VGRKPKVEQL (SEQ ID NO: 141).

Notably, many of the binding regions identified in structural studies using four representative isoform-selective TGFβ1 antibodies are found to be overlapping, pointing to certain regions within the proTGFβ1 complex that may be particularly important in maintaining latency of the proTGFβ1 complex. Thus, advantageously, antibodies or fragments thereof may be selected at least in part on the basis of their binding region(s) that include the overlapping portions identified across multiple inhibitors described herein. These overlapping portions of binding regions include, for example, SPPSQGEVPPGPLPEAVL (SEQ ID NO: 165), WKWIHEPKGYHANF (SEQ ID NO: 166), and PGPLPEAVL (SEQ ID NO: 167). Thus, the high-affinity, isoform-selective TGFβ1 inhibitor according to the present disclosure may bind a proTGFβ1 complex (e.g., human LLCs) at an epitope that comprises one or more amino acid residues of SPPSQGEVPPGPLPEAVL (SEQ ID NO: 165), WKWIHEPKGYHANF (SEQ ID NO: 166), and/or PGPLPEAVL (SEQ ID NO: 167).

Thus, any of the antibody or antigen-binding fragment encompassed by the present disclosure, such as antibodies or fragments of Categories 1 through 5 disclosed herein, may bind one or more of the binding regions identified herein. Such antibodies may be used in the treatment of a TGFβ1 indication in a subject as described herein. Accordingly, selection of an antibody or antigen-binding fragment thereof suitable for therapeutic use in accordance with the present disclosure may include identifying or selecting an antibody or a fragment thereof that binds SPPSQGEVPPGPLPEAVL (SEQ ID NO: 165), WKWIHEPKGYHANF (SEQ ID NO: 166), PGPLPEAVL (SEQ ID NO: 167), or any portion(s) thereof.

Non-limiting examples of protein domains or motifs of human proTGFβ1 as previously described (WO 2014/182676) are provided in Table 9.

TABLE 9
Select protein domains/motifs of human TGFβ1-related polypeptides

| Human TGFβ1 | | SEQ |
|---|---|---|
| domain/module | Amino Acid Sequence | ID NO |
| Latency Associated | LSTCKTIDMELVKRKRIEAIRGQILSKLRLASPPSQGEVPPGPLPEAVLA | 119 |
| Peptide (LAP) | LYNSTRDRVAGESAEPEPEPEADYYAKEVTRVLMVETHNEIYDKFKQS | |
| (prodomain) | THSIYMFFNTSELREAVPEPVLLSRAELRLLRLKLKVEQHVELYQKYSN | |
| | NSWRYLSNRLLAPSDSPEWLSFDVTGVVRQWLSRGGEIEGFRLSAHC | |
| | SCDSRDNTLQVDINGFTTGRRGDLATIHGMNRPFLLLMATPLERAQHL | |
| | QSSRHRR | |
| | (“First binding region” is underlined) | |
| Straight Jacket | LSTCKTIDMELVKRKRIEAIRGQILSKLRLASPPSQGEVPPGPLP | 120 |
| | (“Latency Lasso” is underlined) | |
| Growth Factor | ALDTNYCFSSTEKNCCVRQLYIDFRKDLGWKWIHEPKGYHANFCLGP | 121 |
| Domain | CPYIWSLDTQYSKVLALYNQHNPGASAAPCCVPQALEPLPIVYYVGRK | |
| | PKVEQLSNMIVRSCKCS | |
| | (“Finger-1” and “Finger-2” are underlined, respectively) | |
| Fastener | residues 74-76, YYA | n/a |
| Furin cleavage site | RHRR | 122 |
| Arm | EAVLALYNSTRDRVAGESAEPEPEPEADYYAKEVTRVLMVETHNEIYD | 123 |
| | KFKQSTHSIYMFFNTSELREAVPEPVLLSRAELRLLRLKLKVEQHVELY | |
| | QKYSNNSWRYLSNRLLAPSDSPEWLSFDVTGVVRQWLSRGGEIEGFR | |
| | LSAHCSCDSRDNTLQVDINGFTTGRRGDLATIHGMNRPFLLLMATPLE | |
| | RAQHLQSSRHRR | |
| Finger-1 | CVRQLYIDFRKDLGWKWIHEPKGYHANFC | 124 |
| | (“Second binding region” is underlined) | |
| Finger-2 | CVPQALEPLPIVYYVGRKPKVEQLSNMIVRSCKCS | 125 |
| | (“Third binding region” is underlined) | |
| Residue for | Cys 4 | n/a |
| presenting molecule | | |
| association | | |
| Latency Lasso | LASPPSQGEVPPGPL | 126 |
| | (Portion of the binding regions shared across 4 different | |
| | isoform-selective proTGFβ1 antibodies is underlined) | |
| Extended Latency | LASPPSQGEVPPGPLPEAVLALYNSTR | 127 |
| Lasso | (Portion of the binding regions shared across 4 different | |
| | isoform-selective proTGFβ1 antibodies is underlined) | |
| Alpha-1 Helix | LSTCKTIDMELVKRKRIEAIRGQILSKLR | 128 |
| Alpha-2 Helix | AVLALYNSTR | 129 |
| Trigger Loop | NGFTTGRRGDLATIHGMNRP | 130 |
| Integrin binding | residue 215-217, RGD | n/a |
| Bowtie | CSCDSRDNTLQVD | 131 |

### Safety/Toxicology

The development of TGFβ inhibitors remains challenging due to the need to identify a therapy with the desired pharmacological effects and sufficient therapeutic window, which also eliminates on-target toxicities. The majority of TGFβ inhibitors, including monoclonal antibodies and small molecule kinase inhibitors (SMIs), non-selectively target either multiple TGFβ isoforms or the TGFβ receptor, which mediates signaling from all three TGFβ isoforms. Unfortunately, these inhibitors have not demonstrated promising clinical data in cancer patients mainly due to a lack of efficacy (Akhurst 2017; Cohn 2014; Voelker 2017), an unfavorable safety profile, or both (Tolcher 2017; Volker 2017; Cohn 2014). The toxicities associated with these molecules include cardiovascular abnormalities, epithelial hyperplasia, gastrointestinal abnormalities, and skin lesions. Each of these toxicities have been characterized in multiple animal species (e.g., rodents, dogs, and cynomolgus monkeys) in studies ranging in duration from 1-2 weeks up to 6-months (Lonning 2011; Stauber 2014; Mitra 2020). Amongst these toxicities, the irreversible cardiovascular inflammatory lesions, hemorrhage and hyperplasia in heart valves, and arterial lesions that include the aorta and coronary arteries, are of major concern.

Conventional pan-inhibitors of TGFβ capable of antagonizing multiple isoforms have been known to cause a number of toxicities, including, for example, cardiovascular toxicities (cardiac lesions, most notably valvulopathy) reported across multiple species including dogs and rats. These include, hyperplasia in aortic valve, right AV valve, and left AV valve; inflammation in aortic valve, left AV valve, and ascending aorta; hemorrhage in ascending aorta, aortic valve and left AV valve; connective tissue degeneration in ascending aorta (see for example, Strauber et al., (2014) “Nonclinical safety evaluation of a Transforming Growth Factor β receptor I kinase inhibitor in Fischer 344 rats and beagle dogs” J. Clin. Pract 4(3): 1000196).

In addition, neutralizing antibodies that bind all three TGFβ isoforms have been associated with certain epithelial toxicities observed across multiple species, some of which are summarized below.

TABLE 10
Epithelial toxicities associated with pan-inhibitors of TGFβ

| | Mice | Cyno | Human |
|---|---|---|---|
| Toxicities | Hyperplasia and | Hyperplasia of gingiva, | Gigival bleeding |
| | inflammation of tongue, | nasal epithelium, and | Epistaxis |
| | gingiva, and esophagus. | bladder | Headache |
| | Findings not reversible | Anemia lead to | Fatigue |
| | (12 wk recovery) | cessation of treatment | Various skin disorders, |
| | | Changes were | including |
| | | reversible (except | keratoacanthomas (KA), |
| | | bladder) | hyperkeratosis, |
| | | | cutaneous SCC, and |
| | | | basal cell carcinoma |
| Drug/Dose/ | 1D11 | GC1008 | GC1008 |
| Duration | Dosing: 50 mg/kg (3x/week) | Dosing: 10 and 50 mg/kg | Dose: 0.1, 0.3, 1, |
| | Duration: 9-12 weeks | Duration: 6 months | 3, 10, 15 mg/kg |
| | | | Duration: 4 monthly doses |
| Exposure | Serum conc. = 1-2 mg/mL | Not disclosed | Half life: 21.7 d |
| | (over 4-12 weeks) | | DN Cmax ~(350 ng/mL)mg |

*Vitsky et. al., Am. J Pathology vol. 174, 2009; and Lonning et. al., Current Pharmaceutical Biotech 12, 2176-2189, 2011

Building upon the earlier recognition by the applicant of the present disclosure (see PCT/US2017/021972) that lack of isoform-specificity of conventional TGFβ antagonists may underlie the source of toxicities associated with TGFβ inhibition, the present inventors sought to further achieve broad-spectrum TGFβ1 inhibition for treating various diseases that manifest multifaceted TGFβ1 dysregulation, while maintaining the safety/tolerability aspect of isoform-selective inhibitors.

In clinical setting, therapeutic benefit is achieved only when the minimum effective concentrations (MEC) of a drug (e.g., monoclonal antibody) are below the minimum toxic concentrations (MTC) of the drug. This was not achieved with most, if not all, conventional pan-inhibitors of TGFβ, which in fact appeared to cause dose-limiting toxicities. Applicant's previous work described isoform-selective inhibitors of TGFβ1 that showed markedly improved safety profile, as compared to conventional pan-inhibitors, such as small molecule receptor antagonists and neutralizing antibodies. WO 2017/156500 disclosed an isoform-selective inhibitor of TGFβ1 activation, which, when administered at a dose of up to 100 mg/kg per week for 4 weeks in rats, no test article-related toxicities were observed, establishing the NOAEL for the antibody as the highest dose tested, i.e., 100 mg/kg. Applicant's subsequent work also showed that an antibody with enhanced function also showed the equivalent safety profiles. Here, one of the objectives was to identify antibodies with even higher affinities and potencies, but with at least the same or equivalent levels of safety.

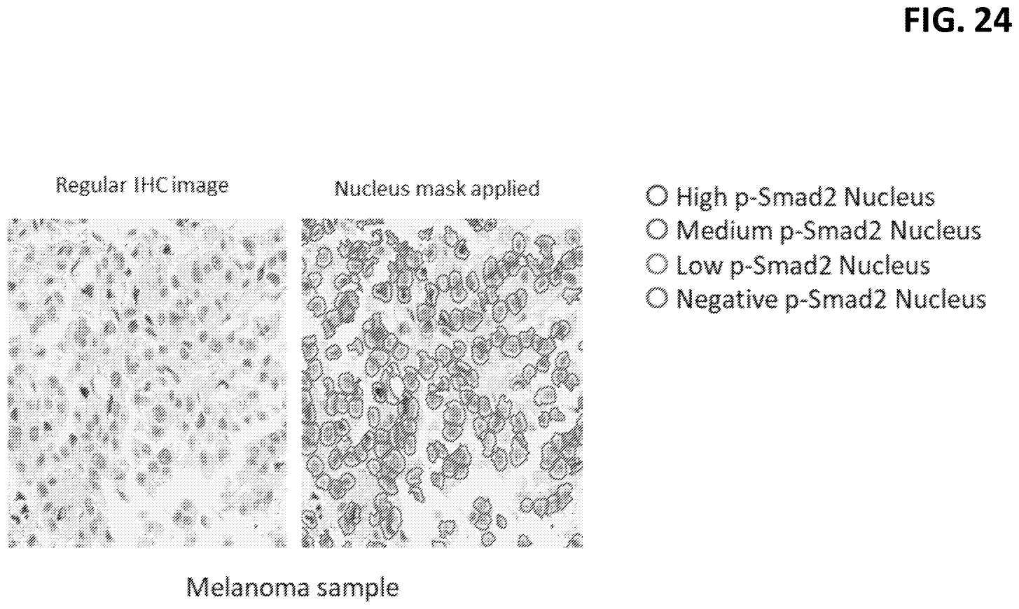

Results from four-week rat toxicology studies were previously disclosed in PCT/US2019/041373, see, e.g., data provided in FIG. 24B. Two isoform-selective TGFβ1 inhibitors (Ab3 and Ab6) were tested in separate studies, together with a small molecule ALK5 inhibitor and a monoclonal neutralizing antibody as control. No test article-related toxicities were noted with either of the isoform-selective antibodies, while the non-selective inhibitors as expected caused a variety of adverse events consistent with published studies. In contrast to treatments that broadly block TGFβ signaling, Ab6 showed no cardiac toxicities in a 4-week, non-GLP pilot toxicology study in rats, suggesting that selective inhibition of the TGFβ1 isoform may have an improved safety profile compared to pan-TGFβ inhibitors. Moreover, Ab6 was shown to be safe (e.g., no observed adverse events) at a dose level as high as 300 mg/kg in cynomolgus monkeys when dosed weekly for 4 weeks. Since Ab6 has been shown to be efficacious in a number of in vivo models at a dose as low as 3 mg/kg, this offers an up to 100-fold of a therapeutic window. Importantly, this demonstrates that high potency does not have to mean greater risk of toxicity. Without wishing to be bound by a particular theory, it is contemplated that the highly selective nature of the antibodies disclosed herein likely account for the lack of observed toxicities.

Thus, in some embodiments, the novel antibody according to the present disclosure has the maximally tolerated dose (MTD) of >100 mg/kg when dosed weekly for at least 4 weeks. In some embodiments, the novel antibody according to the present disclosure has the no-observed-adverse-effect level (NOAEL) of up to 100 mg/kg when dosed weekly for at least 4 weeks. Suitable animal models to be used for conducting safety/toxicology studies for TGFβ inhibitors and TGFβ1 inhibitors include, but are not limited to: rats, dogs, cynos, and mice. In certain embodiments, the minimum effective amount of the antibody based on a suitable preclinical efficacy study is below the NOAEL. More preferably, the minimum effective amount of the antibody is about one-third or less of the NOAEL. In certain embodiments, the minimum effective amount of the antibody is about one-sixth or less of the NOAEL. In some embodiments, the minimum effective amount of the antibody is about one-tenth or less of the NOAEL.

In some embodiments, the disclosure encompasses an isoform-selective antibody capable of inhibiting TGFβ1 signaling, which, when administered to a subject, does not cause cardiovascular or known epithelial toxicities at a dose effective to treat a TGFβ31-related indication. In some embodiments, the antibody has a minimum effective amount of about 3-10 mg/kg administered weekly, biweekly or monthly. Preferably, the antibody causes no to minimum toxicities at a dose that is at least six-times the minimum effective amount (e.g., a six-fold therapeutic window). More preferably, the antibody causes no to minimum toxicities at a dose that is at least ten-times the minimum effective amount (e.g., a ten-fold therapeutic window). Even more preferably, the antibody causes no to minimum toxicities at a dose that is at least fifteen-times the minimum effective amount (e.g., a fifteen-fold therapeutic window).

Therapeutic agents that engage immune cells pose the potential risk of activating immune cells when administered to patients. In selecting a TGFβ inhibitor for therapeutic use, it is therefore important to determine or confirm that a candidate inhibitor does not trigger a proinflammatory cytokine response (e.g., cytokine release) in human peripheral blood mononuclear cells (PBMCs). Proinflammatory cytokines include, for example, IFNγ, IL-2, IL-1, TNFβ, CCL2 and IL-6. In some embodiments, acceptable levels of cytokine release triggered by a test agent (candidate inhibitor) are within 2.5-fold of the response as compared to vehicle control (e.g., IgG).

Accordingly, the present disclosure provides a TGFβ inhibitor for use in the treatment of a TGFβ-related condition (e.g., cancer, myelofibrosis, fibrosis, etc.) in a human patient, which includes i) selection of a TGFβ inhibitor, which has been shown not to trigger unsafe levels of proinflammatory cytokine release in human PBMCs; and, ii) administration of a composition comprising a therapeutically effective amount of the TGFβ inhibitor to the patient, to treat the condition, In some embodiments, the TGFβ inhibitor does not trigger unsafe levels of cytokine release from human PBMCs at an amount that is at least three times the therapeutically effective amount. Preferably, at least five times the therapeutically effective amount of the TGFβ inhibitor does not cause unsafe levels of cytokine release in human PBMCs.

Human platelets have been reported to express latent TGFβ1. Pharmacological intervention that targets platelets may cause unwanted effects on platelet function, such as platelet aggregation and activation, which could result in blood coagulation dysregulation. Therefore, it is important to determine or confirm that a candidate inhibitor does not cause unwanted platelet activation or interfere with the normal function of platelets.

Accordingly, the present disclosure provides a TGFβ inhibitor for use in the treatment of a TGFβ-related condition (e.g., cancer, myelofibrosis, fibrosis, etc.) in a human patient, which includes i) selection of a TGFβ inhibitor, which has been shown not to cause platelet aggregation or activation; and, ii) administration of a composition comprising a therapeutically effective amount of the TGFβ inhibitor to the patient, to treat the condition, In some embodiments, the TGFβ inhibitor does not cause spontaneous or ADP-induced platelet activation in a dose-dependent manner at an amount that is at least three times the therapeutically effective amount. Preferably, at least five times the therapeutically effective amount of the TGFβ inhibitor does not cause platelet activation. In certain embodiments, the TGFβ inhibitor does not inhibit ADP-induced platelet activation in a dose-dependent manner at an amount that is at least three times the therapeutically effective amount. Preferably, at least five times the therapeutically effective amount of the TGFβ inhibitor does not inhibit platelet activation.

The present disclosure includes a TGFβ inhibitor for use in the treatment of cancer in a human patient, wherein the treatment comprises: i) selecting a TGFβ inhibitor shown to be both efficacious and safe in a preclinical model(s), and, ii) administering to the human patient an effective dose of the TGFβ inhibitor, wherein optionally the TGFβ inhibitor is effective to reduce tumor burden when used in conjunction with a checkpoint inhibitor, wherein further optionally the TGFβ inhibitor does not trigger platelet activation in human blood samples and does not cause inflammatory cytokine release in PBMCs at doses greater than a minimum efficacious dose; and, further optionally the TGFβ inhibitor does not cause unacceptable adverse events as evaluated in a standard toxicology study in one or more preclinical models in which NOAEL is at least 10 times the minimum efficacious dose.

Thus, selection of an antibody or an antigen-binding fragment thereof for therapeutic use may include: selecting an antibody or antigen-binding fragment that meets the criteria of one or more of Categories 1-5 described herein; carrying out an in vivo efficacy study in a suitable preclinical model to determine an effective amount of the antibody or the fragment; carrying out an in vivo safety/toxicology study in a suitable model to determine an amount of the antibody that is safe or toxic (e.g., MTD, NOAEL, cytokine release, effects on platelets, or any art-recognized parameters for evaluating safety/toxicity); and, selecting the antibody or the fragment that provides at least a three-fold therapeutic window (preferably 6-fold, more preferably a 10-fold therapeutic window, even more preferably a 15-fold therapeutic window). In preferred embodiments, the in vivo efficacy study is carried out in two or more suitable preclinical models that recapitulate human conditions. In some embodiments, such preclinical models comprise TGFβ31-positive cancer, which may optionally comprise an immunosuppressive tumor. The immunosuppressive tumor may be resistant to a cancer therapy such as CBT, chemotherapy and radiation therapy (such as a radiotherapeutic agent). In some embodiments, the preclinical models are selected from MBT-2, Cloudman S91 and EMT6 tumor models.

The selected antibody or the fragment may be used in the manufacture of a pharmaceutical composition comprising the antibody or the fragment. Such pharmaceutical composition may be used in the treatment of a TGFβ1 indication in a subject as described herein. For example, the TGFβ1 indication may be a proliferative disorder and/or a fibrotic disorder.

### Mechanism of Action

Antibodies of the present disclosure that are useful as therapeutics are inhibitory antibodies of TGFβ1. Further, the antibodies are activation inhibitors, that is, the antibodies block the activation step of TGFβ1, rather than directly chasing after already activated growth factor.

In a broad sense, the term “inhibiting antibody” refers to an antibody that antagonizes or neutralizes the target function, e.g., growth factor activity. Advantageously, certain inhibitory antibodies of the present disclosure are capable of inhibiting mature growth factor release from a latent complex, thereby reducing growth factor signaling. Inhibiting antibodies include antibodies targeting any epitope that reduces growth factor release or activity when associated with such antibodies. Such epitopes may lie on the prodomains of TGFβ proteins (e.g., TGFβ1), growth factors or other epitopes that lead to reduced growth factor activity when bound by antibody. Inhibiting antibodies of the present disclosure include, but are not limited to, TGFβ1-inhibiting antibodies. In some embodiments, inhibitory antibodies of the present disclosure specifically bind a combinatory epitope, i.e., an epitope formed by two or more components/portions of an antigen or antigen complex. For example, a combinatorial epitope may be formed by contributions from multiple portions of a single protein, i.e., amino acid residues from more than one non-contiguous segments of the same protein. Alternatively, a combinatorial epitope may be formed by contributions from multiple protein components of an antigen complex. In some embodiments, inhibitory antibodies of the present disclosure specifically bind a conformational epitope (or conformation-specific epitope), e.g., an epitope that is sensitive to the three-dimensional structure (i.e., conformation) of an antigen or antigen complex.

Traditional approaches to antagonizing TGFβ signaling have been to i) directly neutralize the mature growth factor after it has already become active so as to deplete free ligands (e.g., released from its latent precursor complex) that are available for receptor binding; ii) employ soluble receptor fragments capable of sequestering free ligands (e.g., so-called ligand traps); or, iii) target its cell-surface receptor(s) to block ligand-receptor interactions. Each of these conventional approaches requires the antagonist to compete against endogenous counterparts. Moreover, the first two approaches (i and ii) above target the active ligand, which is a transient species. Therefore, such antagonist must be capable of kinetically outcompeting the endogenous receptor during the brief temporal window. The third approach may provide a more durable effect in comparison but inadvertently results in unwanted inhibitory effects (hence possible toxicities) because many growth factors (e.g., up to ˜20) signal via the same receptor(s).

To provide solutions to these drawbacks, and to further enable greater selectivity and localized action, the mechanism of action underlining the inhibitory antibodies such as those described herein acts upstream of TGFβ1 activation and ligand-receptor interaction. Thus, it is contemplated that high-affinity, isoform-specific, context-independent inhibitors of TGFβ1 suitable for carrying outthe present disclosure should preferably targetthe inactive (e.g., latent) precursor TGFβ1 complex (e.g., a complex comprising pro/latent TGFβ1) priorto its activation, in order to block the activation step at its source (such as in a disease microenvironment, e.g., TME). According to certain embodiments of the disclosure, such inhibitors target with equivalent affinities both ECM-associated and cell surface-tethered pro/latent TGFβ1 complexes, rather than free ligands that are transiently available for receptor binding.

Advantages of locally targeting tissue/cell-tethered complex at the source, as opposed to soluble active species (i.e., mature growth factors after being released from the source), are further supported by a recent study. Ishihara et al., (Sci. Transl. Med. 11, eaau3259 (2019) “Targeted antibody and cytokine cancer immunotherapies through collagen affinity”) reported that when systemically administered drugs are targeted to the tumor sites by conjugating with a collagen-binding moiety, they were able to enhance anti-tumor immunity and reduce treatment-related toxicities, as compared to non-targeted counterparts.

The mechanism of action achieved by the antibodies of the present disclosure may further contribute to enhanced durability of effect, as well as overall greater potency and safety.

Interestingly, these antibodies may exert additional inhibitory activities toward cell-associated TGFβ1 (LRRC33-proTGFβ1 and GARP-proTGFβ1). Applicant has found that LRRC33-binding antibodies tend to become internalized upon binding to cell-surface LRRC33. Whether the internalization is actively induced by antibody binding, or alternatively, whether this phenomenon results from natural (e.g., passive) endocytic activities of macrophages is unclear. However, the high-affinity, isoform-selective TGFβ1 inhibitor, Ab6, is capable of becoming rapidly internalized in cells transfected with LRRC33 and proTGFβ1, and the rate of internalization achieved with Ab6 is significantly higher than that with a reference antibody that recognizes cell-surface LRRC33. Similar results are obtained from primary human macrophages. These observations raise the possibility that Ab6 can induce internalization upon binding to its target, LRRC33-proTGFβ1, thereby removing the LRRC33-containing complexes from the cell surface. At the disease loci, this may reduce the availability of activatable latent LRRC33-proTGFβ1 levels. Therefore, the isoform-selective TGFβ1 inhibitors may inhibit the LRRC33 arm of TGFβ1 via two parallel mechanisms of action: i) blocking the release of mature growth factor from the latent complex; and, ii) removing LRRC33-proTGFβ1 complexes from cell-surface via internalization. It is possible that similar inhibitory mechanisms of action may apply to GARP-proTGFβ1.

In some embodiments, the antibody is a pH-sensitive antibody that binds its antigen with higher affinity at a neutral pH (such as pH of around 7) than at an acidic pH (such as pH of around 5). Such antibodies may have higher dissociation rates at acidic conditions than neutral or physiological conditions. For example, the ratio between dissociation rates measured at an acidic pH and dissociation rates measured at neutral pH (e.g., K_{off} at pH5 over K_{off} at pH 7) may be at least 1.2. Optionally, the ratio is at least 1.5. In some embodiments, the ratio is at least 2. Such pH-sensitive antibodies may be useful as recycling antibodies. Upon target engagement on cell surface, the antibody may trigger antibody-dependent internalization of (hence removal of) membrane-bound proTGFβ1 complexes (associated with LRRC33 or GARP). Subsequently, in an acidic intracellular compartment such as lysosome, the antibody-antigen complex dissociates, and the free antibody may be transported back to the extracellular domain.

Thus, in some embodiments, selection of an antibody or an antigen-binding fragment for therapeutic use may be in part based on the ability to induce antibody-dependent internalization and/or pH-dependency of the antibody.

### Antigen Complexes and Components Thereof

The novel antibodies of the present disclosure specifically bind each of the four known human large latency complexes (e.g., hLTBP1-proTGFβ1, hLTBP3-proTGFβ1, hGARP-proTGFβ1 and hLRRC33-proTGFβ1), selectively inhibits TGFβ1 activation.

Screening (e.g., identification and selection) of such antibodies involves the use of suitable antigen complexes, which are typically recombinantly produced. Useful protein components that may comprise such antigen complexes are provided, including TGFβ isoforms and related polypeptides, fragments and variants, presenting molecules (e.g., LTBPs, GARP, LRRC33) and related polypeptides, fragments and variants. These components may be expressed, purified, and allowed to form a protein complex (such as large latent complexes), which can be used in the process of antibody screening. The screening may include positive selection, in which desirable binders are selected from a pool or library of binders and non-binders, and negative selection, in which undesirable binders are removed from the pool. Typically, at least one matrix-associated complex (e.g., LTBP1-proTGFβ1 and/or LTBP1-proTGFβ1) and at least one cell-associated complex (e.g., GARP-proTGFβ1 and/or LRRC33-proTGFβ1) are included for positive screening to ensure that binders being selected have affinities for both such biological contexts.

In some embodiments, the TGFβ1 comprises a naturally occurring mammalian amino acid sequence. In some embodiment, the TGFβ1 comprises a naturally occurring human amino acid sequence. In some embodiments, the TGFβ1 comprises a human, a monkey, a rat or a mouse amino acid sequence. In some embodiments, an antibody, or antigen binding portion thereof, described herein does not specifically bind to TGFβ2. In some embodiments, an antibody, or antigen binding portion thereof, described herein does not specifically bind to TGFβ3. In some embodiments, an antibody, or antigen binding portion thereof, described herein does not specifically bind to TGFβ2 or TGFβ3. In some embodiments, an antibody, or antigen binding portion thereof, described herein specifically binds to a TGFβ1 comprising the amino acid sequence set forth in SEQ ID NO: 23. The amino acid sequences of TGFβ2, and TGFβ3 amino acid sequence are set forth in SEQ ID NOs: 27 and 21, respectively. In some embodiments, an antibody, or antigen binding portion thereof, described herein specifically binds to a TGFβ1 comprising a non-naturally-occurring amino acid sequence (otherwise referred to herein as a non-naturally-occurring TGFβ1). For example, a non-naturally-occurring TGFβ1 may comprise one or more recombinantly generated mutations relative to a naturally-occurring TGFβ1 amino acid sequence. In some embodiments, a TGFβ1, TGFβ2, or TGFβ3 amino acid sequence comprises the amino acid sequence as set forth in SEQ ID NOs: 13-24, as shown in Table 11. In some embodiments, a TGFβ1, TGFβ2, or TGFβ3 amino acid sequence comprises the amino acid sequence as set forth in SEQ ID NOs: 25-32, as shown in Table 12. PGP-36 DNA

TGFβ1 (prodomain + growth factor domain)
(SEQ ID NO: 13)
LSTCKTIDMELVKRKRIEAIRGQILSKLRLASPPSQGEVPPGPLPEAVLALYNSTRDRVAGESAEPEPEPEADYYA
KEVTRVLMVETHNEIYDKFKQSTHSIYMFFNTSELREAVPEPVLLSRAELRLLRLKLKVEQHVELYQKYSNNSWRY
LSNRLLAPSDSPEWLSFDVTGVVRQWLSRGGEIEGFRLSAHCSCDSRDNTLQVDINGFTTGRRGDLATIHGMNR
PFLLLMATPLERAQHLQSSRHRRALDTNYCFSSTEKNCCVRQLYIDFRKDLGWKWIHEPKGYHANFCLGPCPYI
WSLDTQYSKVLALYNQHNPGASAAPCCVPQALEPLPIVYYVGRKPKVEQLSNMIVRSCKCS
TGFβ2 (prodomain + growth factor domain)
(SEQ ID NO: 17)
SLSTCSTLDMDQFMRKRIEAIRGQILSKLKLTSPPEDYPEPEEVPPEVISIYNSTRDLLQEKASRRAAACERERSDE
EYYAKEVYKIDMPPFFPSENAIPPTFYRPYFRIVRFDVSAMEKNASNLVKAEFRVFRLQNPKARVPEQRIELYQILK
SKDLTSPTQRYIDSKVVKTRAEGEWLSFDVTDAVHEWLHHKDRNLGFKISLHCPCCTFVPSNNYIIPNKSEELEAR
FAGIDGTSTYTSGDQKTIKSTRKKNSGKTPHLLLMLLPSYRLESQQTNRRKKRALDAAYCFRNVQDNCCLRPLYI
DFKRDLGWKWIHEPKGYNANFCAGACPYLWSSDTQHSRVLSLYNTINPEASASPCCVSQDLEPLTILYYIGKTPKI
EQLSNMIVKSCKCS
TGFβ3 (prodomain + growth factor domain)
(SEQ ID NO: 21)
SLSLSTCTTLDFGHIKKKRVEAIRGQILSKLRLTSPPEPTVMTHVPYQVLALYNSTRELLEEMHGEREEGCTQENT
ESEYYAKEIHKFDMIQGLAEHNELAVCPKGITSKVFRFNVSSVEKNRTNLFRAEFRVLRVPNPSSKRNEQRIELFQI
LRPDEHIAKQRYIGGKNLPTRGTAEWLSFDVTDTVREWLLRRESNLGLEISIHCPCHTFQPNGDILENIHEVMEIKF
KGVDNEDDHGRGDLGRLKKQKDHHNPHLILMMIPPHRLDNPGQGGQRKKRALDTNYCFRNLEENCCVRPLYID
FRQDLGWKWVHEPKGYYANFCSGPCPYLRSADTTHSTVLGLYNTLNPEASASPCCVPQDLEPLTILYYVGRTPK
VEQLSNMVVKSCKCS

TABLE 11
Exemplary TGFβ1, TGFβ2, and TGFβ3 amino acid sequences

| | | SEQ ID |
|---|---|---|
| Protein | Sequence | NO |
| proTGFβ1 | LSTCKTIDMELVKRKRIEAIRGQILSKLRLASPPSQGEVPPGPLPEAVLAL | 13 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHRRALDTNYCFSSTEKNCCVRQLYIDFRKDLGWKWIHEPKGYHANFC | |
| | LGPCPYIWSLDTQYSKVLALYNQHNPGASAAPCCVPQALEPLPIVYYVG | |
| | RKPKVEQLSNMIVRSCKCS | |
| proTGFβ1 C4S | LSTSKTIDMELVKRKRIEAIRGQILSKLRLASPPSQGEVPPGPLPEAVLAL | 14 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHRRALDTNYCFSSTEKNCCVRQLYIDFRKDLGWKWIHEPKGYHANFC | |
| | LGPCPYIWSLDTQYSKVLALYNQHNPGASAAPCCVPQALEPLPIVYYVG | |
| | RKPKVEQLSNMIVRSCKCS | |
| proTGFβ1 D2G | LSTCKTIDMELVKRKRIEAIRGQILSKLRLASPPSQGEVPPGPLPEAVLAL | 15 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHGALDTNYCFSSTEKNCCVRQLYIDFRKDLGWKWIHEPKGYHANFCL | |
| | GPCPYIWSLDTQYSKVLALYNQHNPGASAAPCCVPQALEPLPIVYYVGR | |
| | KPKVEQLSNMIVRSCKCS | |
| proTGFβ1 C4S D2G | LSTSKTIDMELVKRKRIEAIRGQILSKLRLASPPSQGEVPPGPLPEAVLAL | 16 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHGALDTNYCFSSTEKNCCVRQLYIDFRKDLGWKWIHEPKGYHANFCL | |
| | GPCPYIWSLDTQYSKVLALYNQHNPGASAAPCCVPQALEPLPIVYYVGR | |
| | KPKVEQLSNMIVRSCKCS | |
| proTGFβ2 | SLSTCSTLDMDQFMRKRIEAIRGQILSKLKLTSPPEDYPEPEEVPPEVISI | 17 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKKRALDAAYCFRNVQDN | |
| | CCLRPLYIDFKRDLGWKWIHEPKGYNANFCAGACPYLWSSDTQHSRVL | |
| | SLYNTINPEASASPCCVSQDLEPLTILYYIGKTPKIEQLSNMIVKSCKCS | |
| proTGFβ2 C5S | SLSTSSTLDMDQFMRKRIEAIRGQILSKLKLTSPPEDYPEPEEVPPEVISI | 18 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKKRALDAAYCFRNVQDN | |
| | CCLRPLYIDFKRDLGWKWIHEPKGYNANFCAGACPYLWSSDTQHSRVL | |
| | SLYNTINPEASASPCCVSQDLEPLTILYYIGKTPKIEQLSNMIVKSCKCS | |
| proTGFβ2 C5S D2G | SLSTSSTLDMDQFMRKRIEAIRGQILSKLKLTSPPEDYPEPEEVPPEVISI | 19 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKGALDAAYCFRNVQDNC | |
| | CLRPLYIDFKRDLGWKWIHEPKGYNANFCAGACPYLWSSDTQHSRVLS | |
| | LYNTINPEASASPCCVSQDLEPLTILYYIGKTPKIEQLSNMIVKSCKCS | |
| proTGFβ2 D2G | SLSTCSTLDMDQFMRKRIEAIRGQILSKLKLTSPPEDYPEPEEVPPEVISI | 20 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKGALDAAYCFRNVQDNC | |
| | CLRPLYIDFKRDLGWKWIHEPKGYNANFCAGACPYLWSSDTQHSRVLS | |
| | LYNTINPEASASPCCVSQDLEPLTILYYIGKTPKIEQLSNMIVKSCKCS | |
| proTGFβ3 | SLSLSTCTTLDFGHIKKKRVEAIRGQILSKLRLTSPPEPTVMTHVPYQVLA | 21 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKKRALDTNYCFRNLEENCC | |
| | VRPLYIDFRQDLGWKWVHEPKGYYANFCSGPCPYLRSADTTHSTVLGL | |
| | YNTLNPEASASPCCVPQDLEPLTILYYVGRTPKVEQLSNMVVKSCKCS | |
| proTGFβ3 C7S | SLSLSTSTTLDFGHIKKKRVEAIRGQILSKLRLTSPPEPTVMTHVPYQVLA | 22 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKKRALDTNYCFRNLEENCC | |
| | VRPLYIDFRQDLGWKWVHEPKGYYANFCSGPCPYLRSADTTHSTVLGL | |
| | YNTLNPEASASPCCVPQDLEPLTILYYVGRTPKVEQLSNMVVKSCKCS | |
| proTGFβ3 C7S D2G | SLSLSTSTTLDFGHIKKKRVEAIRGQILSKLRLTSPPEPTVMTHVPYQVLA | 23 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKGALDTNYCFRNLEENCCV | |
| | RPLYIDFRQDLGWKWVHEPKGYYANFCSGPCPYLRSADTTHSTVLGLY | |
| | NTLNPEASASPCCVPQDLEPLTILYYVGRTPKVEQLSNMVVKSCKCS | |
| proTGFβ3 D2G | SLSLSTCTTLDFGHIKKKRVEAIRGQILSKLRLTSPPEPTVMTHVPYQVLA | 24 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKGALDTNYCFRNLEENCCV | |
| | RPLYIDFRQDLGWKWVHEPKGYYANFCSGPCPYLRSADTTHSTVLGLY | |
| | NTLNPEASASPCCVPQDLEPLTILYYVGRTPKVEQLSNMVVKSCKCS | |

TABLE 12
Exemplary non-human amino acid sequences

| | | | SEQ ID |
|---|---|---|---|
| Protein | Species | Sequence | NO |
| proTGFβ1 | Mouse | LSTCKTIDMELVKRKRIEAIRGQILSKLRLASPPSQGEVPPGPLPEAVL | 25 |
| | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRRALDTNYCFSSTEKNCCVRQLYIDFRKDLGWK | |
| | | WIHEPKGYHANFCLGPCPYIWSLDTQYSKVLALYNQHNPGASASPC | |
| | | CVPQALEPLPIVYYVGRKPKVEQLSNMIVRSCKCS | |
| proTGFβ1 | Cyno | LSTCKTIDMELVKRKRIEAIRGQILSKLRLASPPSQGEVPPGPLPEAVL | 26 |
| | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRRALDTNYCFSSTEKNCCVRQLYIDFRKDLGWKWI | |
| | | HEPKGYHANFCLGPCPYIWSLDTQYSKVLALYNQHNPGASAAPCCV | |
| | | PQALEPLPIVYYVGRKPKVEQLSNMIVRSCKCS | |
| TGFβ1 LAP | Mouse | LSTSKTIDMELVKRKRIEAIRGQILSKLRLASPPSQGEVPPGPLPEAVL | 27 |
| C4S | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRR | |
| TGFβ1 LAP | Cyno | LSTSKTIDMELVKRKRIEAIRGQILSKLRLASPPSQGEVPPGPLPEAVL | 28 |
| C4S | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRR | |
| proTGFβ1 | Mouse | LSTSKTIDMELVKRKRIEAIRGQILSKLRLASPPSQGEVPPGPLPEAVL | 29 |
| C4S D2G | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHGALDTNYCFSSTEKNCCVRQLYIDFRKDLGWKWI | |
| | | HEPKGYHANFCLGPCPYIWSLDTQYSKVLALYNQHNPGASASPCCV | |
| | | PQALEPLPIVYYVGRKPKVEQLSNMIVRSCKCS | |
| proTGFβ1 | Mouse | LSTSKTIDMELVKRKRIEAIRGQILSKLRLASPPSQGEVPPGPLPEAVL | 30 |
| C4S | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRRALDTNYCFSSTEKNCCVRQLYIDFRKDLGWK | |
| | | WIHEPKGYHANFCLGPCPYIWSLDTQYSKVLALYNQHNPGASASPC | |
| | | CVPQALEPLPIVYYVGRKPKVEQLSNMIVRSCKCS | |
| proTGFβ1 | Cyno | LSTSKTIDMELVKRKRIEAIRGQILSKLRLASPPSQGEVPPGPLPEAVL | 31 |
| C4S | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRRALDTNYCFSSTEKNCCVRQLYIDFRKDLGWKWI | |
| | | HEPKGYHANFCLGPCPYIWSLDTQYSKVLALYNQHNPGASAAPCCV | |
| | | PQALEPLPIVYYVGRKPKVEQLSNMIVRSCKCS | |
| proTGFβ1 | Cyno | LSTSKTIDMELVKRKRIEAIRGQILSKLRLASPPSQGEVPPGPLPEAVL | 32 |
| C4S D2G | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHGALDTNYCFSSTEKNCCVRQLYIDFRKDLGWKWIH | |
| | | EPKGYHANFCLGPCPYIWSLDTQYSKVLALYNQHNPGASAAPCCVP | |
| | | QALEPLPIVYYVGRKPKVEQLSNMIVRSCKCS | |
| LTBP3 | CYNO | GPAGERGAGGGGALARERFKVVFAPVICKRTCLKGQCRDSCQQGS | 33 |
| | | NMTLIGENGHSTDTLTGSGFRVVVCPLPCMNGGQCSSRNQCLCPP | |
| | | DFTGRFCQVPAGGAGGGTGGSGPGLSRAGALSTGALPPLAPEGDS | |
| | | VASKHAIYAVQVIADPPGPGEGPPAQHAAFLVPLGPGQISAEVQAPP | |
| | | PVVNVRVHHPPEASVQVHRIESSNAEGAAPSQHLLPHPKPSHPRPP | |
| | | TQKPLGRCFQDTLPKQPCGSNPLPGLTKQEDCCGSIGTAWGQSKC | |
| | | HKCPQLQYTGVQKPGPVRGEVGADCPQGYKRLNSTHCQDINECAM | |
| | | PGVCRHGDCLNNPGSYRCVCPPGHSLGPSRTQCIADKPEEKSLCF | |
| | | RLVSPEHQCQHPLTTRLTRQLCCCSVGKAWGARCQRCPADGTAAF | |
| | | KEICPAGKGYHILTSHQTLTIQGESDFSLFLHPDGPPKPQQLPESPS | |
| | | QAPPPEDTEEERGVTTDSPVSEERSVQQSHPTATTSPARPYPELIS | |
| | | RPSPPTMRWFLPDLPPSRSAVEIAPTQVTETDECRLNQNICGHGEC | |
| | | VPGPPDYSCHCNPGYRSHPQHRYCVDVNECEAEPCGPGRGICMN | |
| | | TGGSYNCHCNRGYRLHVGAGGRSCVDLNECAKPHLCGDGGFCINF | |
| | | PGHYKCNCYPGYRLKASRPPVCEDIDECRDPSSCPDGKCENKPGS | |
| | | FKCIACQPGYRSQGGGACRDVNECAEGSPCSPGWCENLPGSFRC | |
| | | TCAQGYAPAPDGRSCVDVDECEAGDVCDNGICTNTPGSFQCQCLS | |
| | | GYHLSRDRSHCEDIDECDFPAACIGGDCINTNGSYRCLCPQGHRLV | |
| | | GGRKCQDIDECTQDPGLCLPHGACKNLQGSYVCVCDEGFTPTQDQ | |
| | | HGCEEVEQPHHKKECYLNFDDTVFCDSVLATNVTQQECCCSLGAG | |
| | | WGDHCEIYPCPVYSSAEFHSLCPDGKGYTQDNNIVNYGIPAHRDIDE | |
| | | CMLFGAEICKEGKCVNTQPGYECYCKQGFYYDGNLLECVDVDECL | |
| | | DESNCRNGVCENTRGGYRCACTPPAEYSPAQRQCLSPEEMDVDE | |
| | | CQDPAACRPGRCVNLPGSYRCECRPPWVPGPSGRDCQLPESPAE | |
| | | RAPERRDVCWSQRGEDGMCAGPQAGPALTFDDCCCRQGRGWGA | |
| | | QCRPCPPRGAGSQCPTSQSESNSFWDTSPLLLGKPRRDEDSSEED | |
| | | SDECRCVSGRCVPRPGGAVCECPGGFQLDASRARCVDIDECRELN | |
| | | QRGLLCKSERCVNTSGSFRCVCKAGFARSRPHGACVPQRRR | |
| LTBP3 | Mouse | GPAGERGTGGGGALARERFKVVFAPVICKRTCLKGQCRDSCQQGS | 34 |
| | | NMTLIGENGHSTDTLTGSAFRVVVCPLPCMNGGQCSSRNQCLCPP | |
| | | DFTGRFCQVPAAGTGAGTGSSGPGLARTGAMSTGPLPPLAPEGES | |
| | | VASKHAIYAVQVIADPPGPGEGPPAQHAAFLVPLGPGQISAEVQAPP | |
| | | PVVNVRVHHPPEASVQVHRIEGPNAEGPASSQHLLPHPKPPHPRPP | |
| | | TQKPLGRCFQDTLPKQPCGSNPLPGLTKQEDCCGSIGTAWGQSKC | |
| | | HKCPQLQYTGVQKPVPVRGEVGADCPQGYKRLNSTHCQDINECAM | |
| | | PGNVCHGDCLNNPGSYRCVCPPGHSLGPLAAQCIADKPEEKSLCFR | |
| | | LVSTEHQCQHPLTTRLTRQLCCCSVGKAWGARCQRCPADGTAAFK | |
| | | EICPGKGYHILTSHQTLTIQGESDFSLFLHPDGPPKPQQLPESPSRAP | |
| | | PLEDTEEERGVTMDPPVSEERSVQQSHPTTTTSPPRPYPELISRPSP | |
| | | PTFHRFLPDLPPSRSAVEIAPTQVTETDECRLNQNICGHGQCVPGPS | |
| | | DYSCHCNAGYRSHPQHRYCVDVNECEAEPCGPGKGICMNTGGSY | |
| | | NCHCNRGYRLHVGAGGRSCVDLNECAKPHLCGDGGFCINFPGHYK | |
| | | CNCYPGYRLKASRPPICEDIDECRDPSTCPDGKCENKPGSFKCIAC | |
| | | QPGYRSQGGGACRDVNECSEGTPCSPGWCENLPGSYRCTCAQYE | |
| | | PAQDGLSCIDVDECEAGKVCQDGICTNTPGSFQCQCLSGYHLSRDR | |
| | | SRCEDIDECDFPAACIGGDCINTNGSYRCLCPLGHRLVGGRKCKKDI | |
| | | DECSQDPGLCLPHACENLQGSYVCVCDEGFTLTQDQHGCEEVEQP | |
| | | HHKKECYLNFDDTVFCDSVLATNVTQQECCCSLGAGWGDHCEIYP | |
| | | CPVYSSAEFHSLVPDGKRLHSGQQHCELCIPAHRDIDECILFGAEICK | |
| | | EGKCVNTQPGYECYCKQGFYYDGNLLECVDVDECLDESNCRNGVC | |
| | | ENTRGGYRCACTPPAEYSPAQAQCLIPERWSTPQRDVKCAGASEE | |
| | | RTACVWGPWAGPALTFDDCCCRQPRLGTQCRPCPPRGTGSQCPT | |
| | | SQSESNSFWDTSPLLLGKSPRDEDSSEEDSDECRCVSGRCVPRPG | |
| | | GAVCECPGGFQLDASRARCVDIDECRELNQRGLLCKSERCVNTSG | |
| | | SFRCVCKAGFTRSRPHGPACLSAAADDAAIAHTSVIDHRGYFH | |
| LTBP1S | Cyno | NHTGRIKVVFTPSICKVTCTKGSCQNSCEKGNTTTLISENGHAADTLT | 35 |
| | | ATNFRVVLCHLPCMNGGQCSSRDKCQCPPNFTGKLCQIPVHGASV | |
| | | PKLYQHSQQPGKALGTHVIHSTHTLPLTVTSQQGVKVKFPPNIVNIH | |
| | | VKHPPEASVQIHQVSRIDGPTGQKTKEAQPGQSQVSYQGLPVQKTQ | |
| | | TIHSTYSHQQVIPHVYPVAAKTQLGRCFQETIGSQCGKALPGLSKQE | |
| | | DCCGTVGTSWGFNKCQKCPKKPSYHGYNQMMECLPGYKRVNNTF | |
| | | CQDINECQLQGVCPNGECLNTMGSYRCTCKIGFGPDPTFSSCVPDP | |
| | | PVISEEKGPCYRLVSSGRQCMHPLSVHLTKQLCCCSVGKAWGPHC | |
| | | EKCPLPGTAAFKEICPGGMGYTVSGVHRRRPIHHHVGKGPVFVKPK | |
| | | NTQPVAKSTHPPPLPAKEEPVEALTFSREHGPGVAEPEVATAPPEK | |
| | | EIPSLDQEKTKLEPGQPQLSPGISTIHLHPQFPVVIEKTSPPVPVEVAP | |
| | | EASTSSASQVIAPTQVTEINECTVNPDICGAGHCINLPVRYTCICYEG | |
| | | YKFSEQQRKCVDIDECTQVQHLCSQGRCENTEGSFLCICPAGFMAS | |
| | | EEGTNCIDVDECLRPDVCGEGHCVNTVGAFRCEYCDSGYRMTQRG | |
| | | RCEDIDECLNPSTCPDEQCVNSPGSYQCVPCTEGFRGWNGQCLDV | |
| | | DECLEPNVCTNGDCSNLEGSYMCSCHKGYTRTPDHKHCKDIDECQ | |
| | | QGNLCVNGQCKNTEGSFRCTCGQGYQLSAAKDQCEDIDECQHHHL | |
| | | CAHGQCRNTEGSFQCVCDQGYRASGLGDHCEDINECLEDKSVCQR | |
| | | GDCINTAGSYDCTCPDGFQLDDNKTCQDINECEHPGLCGPQGECL | |
| | | NTEGSFHCVCQQGFSISADGRTCEDIDECVNNTVCDSHGFCDNTAG | |
| | | SFRCLCYQGFQAPQDGQGCVDVNECELLSGVCGEAFCENVEGSFL | |
| | | CVCADENQEYSPMTGQCRSRTSTDLDVEQPKEEKKECYYNLNDAS | |
| | | LCDNVLAPNVTKQECCCTSGAGWGDNCEIFPCPVLGTAEFTEMCPK | |
| | | GKGFVPAGESSSEAGGENYKDADECLLFGQEICKNGFCLNTRPGYE | |
| | | CYCKQGTYYDPVKLQCFDMDECQDPSSCIDGQCVNTEGSYNCFCT | |
| | | HPMVLDASEKRCIRPAESNEQIEETDVYQDLCWEHLSDEYVCSRPL | |
| | | VGKQTTYTECCCLYGEAWGMQCALCPMKDSDDYAQLCNIPVTGRR | |
| | | QPYGRDALVDFSEQYAPEADPYFIQDRFLNSFEELQAEECGILNGCE | |
| | | NGRCVRVQEGYTCDCFDGYHLDTAKMTCVDVNECDELNNRMSLCK | |
| | | NAKCINTEGSYKCLCLPGYVPSDKPNYCTPLNTALNLEKDSDLE | |
| LTBP1S | mouse | NHTGRIKVVFTPSICKVTCTKGNCQNSCQKGNTTTLISENGHAADTL | 36 |
| | | TATNFRVVICHLPCMNGGQCSSRDKCQCPPNFTGKLCQIPVLGASM | |
| | | PKLYQHAQQQGKALGSHVIHSTHTLPLTMTSQQGVKVKFPPNIVNIH | |
| | | VKHPPEASVQIHQVSRIDSPGGQKVKEAQPGQSQVSYQGLPVQKT | |
| | | QTVHSTYSHQQLIPHVYPVAAKTQLGRCFQETIGSQCGKALPGLSK | |
| | | QEDCCGTVGTSWGFNKCQKCPKKQSYHGYTQMMECLQGYKRVN | |
| | | NTFCQDINECQLQGVCPNGECLNTMGSYRCSCKMGFGPDPTFSSC | |
| | | VPDPPVISEEKGPCYRLVSPGRHCMHPLSVHLTKQICCCSVGKAWG | |
| | | PHCEKCPLPGTAAFKEICPGGMGYTVSGVHRRRPIHQHIGKEAVYV | |
| | | KPKNTQPVAKSTHPPPLPAKEEPVEALTSSWEHGPRGAEPEVVTAP | |
| | | PEKEIPSLDQEKTRLEPGQPQLSPGVSTIHLHPQFPVVVEKTSPPVP | |
| | | VEVAPEASTSSASQVIAPTQVTEINECTVNPDICGAGHCINLPVRYTC | |
| | | ICYEGYKFSEQLRKCVDIDECAQVRHLCSQGRCENTEGSFLCVCPA | |
| | | GFMASEEGTNCIDVDECLRPDMCRDGRCINTAGAFRCEYCDSGYR | |
| | | MSRRGYCEDIDECLKPSTCPEEQCVNTPGSYQCVPCTEGFRGWNG | |
| | | QCLDVDECLQPKVCTNGSCTNLEGSYMCSCHRGYSPTPDHRHCQ | |
| | | DIDECQQGNLCMNGQCRNTDGSFRCTCGQGYQLSAAKDQCEDIDE | |
| | | CEHHHLCSHGQCRNTEGSFQCVCNQGYRASVLGDHCEDINECLED | |
| | | SSVCQGGDCINTAGSYDCTCPDGFQLNDNKGCQDINECAQPGLCG | |
| | | SHGECLNTQGSFHCVCEQGFSISADGRTCEDIDECVNNTVCDSHGF | |
| | | CDNTAGSFRCLCYQGFQAPQDGQGCVDVNECELLSGVCGEAFCE | |
| | | NVEGSFLCVCADENQEYSPMTGQCRSRVTEDSGVDRQPREEKKEC | |
| | | YYNLNDASLCDNVLAPNVTKQECCCTSGAGWGDNCEIFPCPVQGT | |
| | | AEFTEMCPRGKGLVPAGESSYDTGGENYKDADECLLFGEEICKNGY | |
| | | CLNTQPGYECYCKQGTYYDPVKLQCFDMDECQDPNSCIDGQCVNT | |
| | | EGSYNCFCTHPMVLDASEKRCVQPTESNEQIEETDVYQDLCWEHLS | |
| | | EEYVCSRPLVGKQTTYTECCCLYGEAWGMQCALCPMKDSDDYAQL | |
| | | CNIPVTGRRRPYGRDALVDFSEQYGPETDPYFIQDRFLNSFEELQAE | |
| | | ECGILNGCENGRCVRVQEGYTCDCFDGYHLDMAKMTCVDVNECSE | |
| | | LNNRMSLCKNAKCINTEGSYKCLCLPGYIPSDKPNYCTPLNSALNLD | |
| | | KESDLE | |
| GARP | mouse | ISQRREQVPCRTVNKEALCHGLGLLQVPSVLSLDIQALYLSGNQLQSI | 37 |
| | | LVSPLGFYTALRHLDLSDNQISFLQAGVFQALPYLEHLNLAHNRLAT | |
| | | GMALNSGGLGRLPLLVSLDLSGNSLHGNLVERLLGETPRLRTLSLAE | |
| | | NSLTRLARHTFWGMPAVEQLDLHSNVLMDIEDGAFEALPHLTHLNLS | |
| | | RNSLTCISDFSLQQLQVLDLSCNSIEAFQTAPEPQAQFQLAWLDLRE | |
| | | NKLLHFPDLAVFPRLIYLNVSNNLIQLPAGLPRGSEDLHAPSEGWSA | |
| | | SPLSNPSRNASTHPLSQLLNLDLSYNEIELVPASFLEHLTSLRFLNLS | |
| | | RNCLRSFEARQVDSLPCLVLLDLSHNVLEALELGTKVLGSLQTLLLQ | |
| | | DNALQELPPYTFASLASLQRLNLQGNQVSPCGGPAEPGPPGCVDFS | |
| | | GIPTLHVLNMAGNSMGMLRAGSFLHTPLTELDLSTNPGLDVATGALV | |
| | | GLEASLEVLELQGNGLTVLRVDLPCFLRLKRLNLAENQLSHLPAWTR | |
| | | AVSLEVLDLRNNSFSLLPGNAMGGLETSLRRLYLQGNPLSCCGNGW | |
| | | LAAQLHQGRVDVDATQDLICRFGSQEELSLSLVRPEDCEKGGLKNV | |
| | | NLILLLSFTLVSAIVLTTLATICFLRRQKLSQQYKA | |
| SGARP | mouse | ISQRREQVPCRTVNKEALCHGLGLLQVPSVLSLDIQALYLSGNQLQSI | 38 |
| | | LVSPLGFYTALRHLDLSDNQISFLQAGVFQALPYLEHLNLAHNRLAT | |
| | | GMALNSGGLGRLPLLVSLDLSGNSLHGNLVERLLGETPRLRTLSLAE | |
| | | NSLTRLARHTFWGMPAVEQLDLHSNVLMDIEDGAFEALPHLTHLNLS | |
| | | RNSLTCISDFSLQQLQVLDLSCNSIEAFQTAPEPQAQFQLAWLDLRE | |
| | | NKLLHFPDLAVFPRLIYLNVSNNLIQLPAGLPRGSEDLHAPSEGWSA | |
| | | SPLSNPSRNASTHPLSQLLNLDLSYNEIELVPASFLEHLTSLRFLNLS | |
| | | RNCLRSFEARQVDSLPCLVLLDLSHNVLEALELGTKVLGSLQTLLLQ | |
| | | DNALQELPPYTFASLASLQRLNLQGNQVSPCGGPAEPGPPGCVDFS | |
| | | GIPTLHVLNMAGNSMGMLRAGSFLHTPLTELDLSTNPGLDVATGALV | |
| | | GLEASLEVLELQGNGLTVLRVDLPCFLRLKRLNLAENQLSHLPAWTR | |
| | | AVSLEVLDLRNNSFSLLPGNAMGGLETSLRRLYLQGNPLSCCGNGW | |
| | | LAAQLHQGRVDVDATQDLICRFGSQEELSLSLVRPEDCEKGGLKNV | |
| | | N | |

In some embodiments, antigenic protein complexes (e.g., a LTBP-TGFβ1 complex) may comprise one or more presenting molecules, such as LTBP proteins (e.g., LTBP1, LTBP2, LTBP3, and LTBP4), GARP proteins, LRRC33 proteins, or fragment(s) thereof. Typically, a minimum required fragment suitable for carrying out the embodiments disclosed herein includes at least 50 amino acids, preferably at least 100 amino acids, of a presenting molecule protein, comprising at least two cysteine residues capable of forming disulfide bonds with a proTGFβ1 complex. Specifically, these Cys residues form covalent bonds with Cysteine resides present near the N-terminus of each monomer of the proTGFβ1 complex. In the three-dimensional structure of a proTGFβ1 dimer complex, the N-terminal so-called “Alpha-1 Helix” of each monomer comes in close proximity to each other, setting the distance between the two cysteine residues (one from each helix) required to form productive covalent bonds with a corresponding pair of cysteines present in a presenting molecule (see, for example, Cuende et al., (2015) Sci. Trans. Med. 7: 284ra56). Therefore, when a fragment of a presenting molecule is used to form an LLC in the screening process (e.g., immunization, library screening, identification, and selection), such fragment should include the cysteine residues separated by the right distance, which will allow proper disulfide bond formation with a proTGFβ1 complex in order to preserve correct conformation of the resulting LLC. LTBPs (e.g., LTBP1, LTBP3 and LTBP4), for example, may contain “cysteine-rich domains” to mediate covalent interactions with proTGFβ1.

An antibody, or antigen binding portion thereof, as described herein, is capable of binding to a LTBP1-TGFβ1 complex. In some embodiments, the LTBP1 protein is a naturally-occurring protein or fragment thereof. In some embodiments, the LTBP1 protein is a non-naturally occurring protein or fragment thereof. In some embodiments, the LTBP1 protein is a recombinant protein. Such recombinant LTBP1 protein may comprise LTBP1, alternatively spliced variants thereof and/or fragments thereof. Recombinant LTBP1 proteins may also be modified to comprise one or more detectable labels. In some embodiments, the LTBP1 protein comprises a leader sequence (e.g., a native or non-native leader sequence). In some embodiments, the LTBP1 protein does not comprise a leader sequence (i.e., the leader sequence has been processed or cleaved). Such detectable labels may include, but are not limited to biotin labels, polyhistidine tags, myc tags, HA tags and/or fluorescent tags. In some embodiments, the LTBP1 protein is a mammalian LTBP1 protein. In some embodiments, the LTBP1 protein is a human, a monkey, a mouse, or a rat LTBP1 protein. In some embodiments, the LTBP1 protein comprises an amino acid sequence as set forth in SEQ ID NOs: 35 and 36 in Table 12. In some embodiments, the LTBP1 protein comprises an amino acid sequence as set forth in SEQ ID NO: 39 in Table 14.

An antibody, or antigen binding portion thereof, as described herein, is capable of binding to a LTBP3-TGFβ1 complex. In some embodiments, the LTBP3 protein is a naturally-occurring protein or fragment thereof. In some embodiments, the LTBP3 protein is a non-naturally occurring protein or fragment thereof. In some embodiments, the LTBP3 protein is a recombinant protein. Such recombinant LTBP3 protein may comprise LTBP3, alternatively spliced variants thereof and/or fragments thereof. In some embodiments, the LTBP3 protein comprises a leader sequence (e.g., a native or non-native leader sequence). In some embodiments, the LTBP3 protein does not comprise a leader sequence (i.e., the leader sequence has been processed or cleaved). Recombinant LTBP3 proteins may also be modified to comprise one or more detectable labels. Such detectable labels may include, but are not limited to biotin labels, polyhistidine tags, myc tags, HA tags and/or fluorescent tags. In some embodiments, the LTBP3 protein is a mammalian LTBP3 protein. In some embodiments, the LTBP3 protein is a human, a monkey, a mouse, or a rat LTBP3 protein. In some embodiments, the LTBP3 protein comprises an amino acid sequence as set forth in SEQ ID NOs: 33 and 34 in Table 12. In some embodiments, the LTBP1 protein comprises an amino acid sequence as set forth in SEQ ID NO: 40 in Table 14.

An antibody, or antigen binding portion thereof, as described herein, is capable of binding to a GARP-TGFβ1 complex. In some embodiments, the GARP protein is a naturally-occurring protein or fragment thereof. In some embodiments, the GARP protein is a non-naturally occurring protein or fragment thereof. In some embodiments, the GARP protein is a recombinant protein. Such a GARP may be recombinant, referred to herein as recombinant GARP. Some recombinant GARPs may comprise one or more modifications, truncations and/or mutations as compared to wild type GARP. Recombinant GARPs may be modified to be soluble. In some embodiments, the GARP protein comprises a leader sequence (e.g., a native or non-native leader sequence). In some embodiments, the GARP protein does not comprise a leader sequence (i.e., the leader sequence has been processed or cleaved). In other embodiments, recombinant GARPs are modified to comprise one or more detectable labels. In further embodiments, such detectable labels may include, but are not limited to biotin labels, polyhistidine tags, flag tags, myc tags, HA tags and/or fluorescent tags. In some embodiments, the GARP protein is a mammalian GARP protein. In some embodiments, the GARP protein is a human, a monkey, a mouse, or a rat GARP protein. In some embodiments, the GARP protein comprises an amino acid sequence as set forth in SEQ ID NOs: 37-38 in Table 12. In some embodiments, the GARP protein comprises an amino acid sequence as set forth in SEQ ID NOs: 41 and 42 in Table 14. In some embodiments, the antibodies, or antigen binding portions thereof, described herein do not bind to TGFβ1 in a context-dependent manner, for example binding to TGFβ1 would only occur when the TGFβ1 molecule was complexed with a specific presenting molecule, such as GARP. Instead, the antibodies, and antigen-binding portions thereof, bind to TGFβ1 in a context-independent manner. In other words, the antibodies, or antigen-binding portions thereof, bind to TGFβ1 when bound to any presenting molecule: GARP, LTBP1, LTBP3, and/or LRRC33.

An antibody, or antigen binding portion thereof, as described herein, is capable of binding to a LRRC33-TGFβ1 complex. In some embodiments, the LRRC33 protein is a naturally-occurring protein or fragment thereof. In some embodiments, the LRRC33 protein is a non-naturally occurring protein or fragment thereof. In some embodiments, the LRRC33 protein is a recombinant protein. Such a LRRC33 may be recombinant, referred to herein as recombinant LRRC33. Some recombinant LRRC33 proteins may comprise one or more modifications, truncations and/or mutations as compared to wild type LRRC33. Recombinant LRRC33 proteins may be modified to be soluble. For example, in some embodiments, the ectodomain of LRRC33 may be expressed with a C-terminal His-tag in order to express soluble LRRC33 protein (sLRRC33; see, e.g., SEQ ID NO: 73). In some embodiments, the LRRC33 protein comprises a leader sequence (e.g., a native or non-native leader sequence). In some embodiments, the LRRC33 protein does not comprise a leader sequence (i.e., the leader sequence has been processed or cleaved). In other embodiments, recombinant LRRC33 proteins are modified to comprise one or more detectable labels. In further embodiments, such detectable labels may include, but are not limited to biotin labels, polyhistidine tags, flag tags, myc tags, HAtags and/or fluorescent tags. In some embodiments, the LRRC33 protein is a mammalian LRRC33 protein. In some embodiments, the LRRC33 protein is a human, a monkey, a mouse, or a rat LRRC33 protein. In some embodiments, the LRRC33 protein comprises an amino acid sequence as set forth in SEQ ID NOs: 72, 73, and 74 in Table 14.

TABLE 13
Exemplary LTBP amino acid sequences

| | | SEQ |
|---|---|---|
| Protein | Sequence | ID NO |
| LTBP1S | NHTGRIKVVFTPSICKVTCTKGSCQNSCEKGNTTTLISENGHAADTLT | 39 |
| | ATNFRVVICHLPCMNGGQCSSRDKCQCPPNFTGKLCQIPVHGASVP | |
| | KLYQHSQQPGKALGTHVIHSTHTLPLTVTSQQGVKVKFPPNIVNIHVK | |
| | HPPEASVQIHQVSRIDGPTGQKTKEAQPGQSQVSYQGLPVQKTQTIH | |
| | STYSHQQVIPHVYPVAAKTQLGRCFQETIGSQCGKALPGLSKQEDCC | |
| | GTVGTSWGFNKCQKCPKKPSYHGYNQMMECLPGYKRVNNTFCQDI | |
| | NECQLQGVCPNGECLNTMGSYRCTCKIGFGPDPTFSSCVPDPPVISE | |
| | EKGPCYRLVSSGRQCMHPLSVHLTKQLCCCSVGKAWGPHCEKCPL | |
| | PGTAAFKEICPGGMGYTVSGVHRRRPIHHHVGKGPVFVKPKNTQPV | |
| | AKSTHPPPLPAKEEPVEALTFSREHGPGVAEPEVATAPPEKEIPSLDQ | |
| | EKTKLEPGQPQLSPGISTIHLHPQFPVVIEKTSPPVPVEVAPEASTSSA | |
| | SQVIAPTQVTEINECTVNPDICGAGHCINLPVRYTCICYEGYRFSEQQ | |
| | RKCVDIDECTQVQHLCSQGRCENTEGSFLCICPAGFMASEEGTNCID | |
| | VDECLRPDVCGEGHCVNTVGAFRCEYCDSGYRMTQRGRCEDIDECL | |
| | NPSTCPDEQCVNSPGSYQCVPCTEGFRGWNGQCLDVDECLEPNVC | |
| | ANGDCSNLEGSYMCSCHKGYTRTPDHKHCRDIDECQQGNLCVNGQ | |
| | CKNTEGSFRCTCGQGYQLSAAKDQCEDIDECQHRHLCAHGQCRNT | |
| | EGSFQCVCDQGYRASGLGDHCEDINECLEDKSVCQRGDCINTAGSY | |
| | DCTCPDGFQLDDNKTCQDINECEHPGLCGPQGECLNTEGSFHCVCQ | |
| | QGFSISADGRTCEDIDECVNNTVCDSHGFCDNTAGSFRCLCYQGFQ | |
| | APQDGQGCVDVNECELLSGVCGEAFCENVEGSFLCVCADENQEYSP | |
| | MTGQCRSRTSTDLDVDVDQPKEEKKECYYNLNDASLCDNVLAPNVT | |
| | KQECCCTSGVGWGDNCEIFPCPVLGTAEFTEMCPKGKGFVPAGESS | |
| | SEAGGENYKDADECLLFGQEICKNGFCLNTRPGYECYCKQGTYYDP | |
| | VKLQCFDMDECQDPSSCIDGQCVNTEGSYNCFCTHPMVLDASEKRC | |
| | IRPAESNEQIEETDVYQDLCWEHLSDEYVCSRPLVGKQTTYTECCCL | |
| | YGEAWGMQCALCPLKDSDDYAQLCNIPVTGRRQPYGRDALVDFSEQ | |
| | YTPEADPYFIQDRFLNSFEELQAEECGILNGCENGRCVRVQEGYTCD | |
| | CFDGYHLDTAKMTCVDVNECDELNNRMSLCKNAKCINTDGSYKCLCL | |
| | PGYVPSDKPNYCTPLNTALNLEKDSDLE | |
| LTBP3 | GPAGERGAGGGGALARERFKVVFAPVICKRTCLKGQCRDSCQQGS | 40 |
| | NMTLIGENGHSTDTLTGSGFRVVVCPLPCMNGGQCSSRNQCLCPPD | |
| | FTGRFCQVPAGGAGGGTGGSGPGLSRTGALSTGALPPLAPEGDSVA | |
| | SKHAIYAVQVIADPPGPGEGPPAQHAAFLVPLGPGQISAEVQAPPPVV | |
| | NVRVHHPPEASVQVHRIESSNAESAAPSQHLLPHPKPSHPRPPTQKP | |
| | LGRCFQDTLPKQPCGSNPLPGLTKQEDCCGSIGTAWGQSKCHKCPQ | |
| | LQYTGVQKPGPVRGEVGADCPQGYKRLNSTHCQDINECAMPGVCR | |
| | HGDCLNNPGSYRCVCPPGHSLGPSRTQCIADKPEEKSLCFRLVSPEH | |
| | QCQHPLTTRLTRQLCCCSVGKAWGARCQRCPTDGTAAFKEICPAGK | |
| | GYHILTSHQTLTIQGESDFSLFLHPDGPPKPQQLPESPSQAPPPEDTE | |
| | EERGVTTDSPVSEERSVQQSHPTATTTPARPYPELISRPSPPTMRWF | |
| | LPDLPPSRSAVEIAPTQVTETDECRLNQNICGHGECVPGPPDYSCHC | |
| | NPGYRSHPQHRYCVDVNECEAEPCGPGRGICMNTGGSYNCHCNRG | |
| | YRLHVGAGGRSCVDLNECAKPHLCGDGGFCINFPGHYKCNCYPGYR | |
| | LKASRPPVCEDIDECRDPSSCPDGKCENKPGSFKCIACQPGYRSQG | |
| | GGACRDVNECAEGSPCSPGWCENLPGSFRCTCAQGYAPAPDGRSC | |
| | LDVDECEAGDVCDNGICSNTPGSFQCQCLSGYHLSRDRSHCEDIDE | |
| | CDFPAACIGGDCINTNGSYRCLCPQGHRLVGGRKCQDIDECSQDPSL | |
| | CLPHGACKNLQGSYVCVCDEGFTPTQDQHGCEEVEQPHHKKECYL | |
| | NFDDTVFCDSVLATNVTQQECCCSLGAGWGDHCEIYPCPVYSSAEF | |
| | HSLCPDGKGYTQDNNIVNYGIPAHRDIDECMLFGSEICKEGKCVNTQ | |
| | PGYECYCKQGFYYDGNLLECVDVDECLDESNCRNGVCENTRGGYR | |
| | CACTPPAEYSPAQRQCLSPEEMDVDECQDPAACRPGRCVNLPGSY | |
| | RCECRPPWVPGPSGRDCQLPESPAERAPERRDVCWSQRGEDGMC | |
| | AGPLAGPALTFDDCCCRQGRGWGAQCRPCPPRGAGSHCPTSQSES | |
| | NSFWDTSPLLLGKPPRDEDSSEEDSDECRCVSGRCVPRPGGAVCEC | |
| | PGGFQLDASRARCVDIDECRELNQRGLLCKSERCVNTSGSFRCVCK | |
| | AGFARSRPHGACVPQRRR | |

TABLE 14
Exemplary GARP and LRRC33 amino acid sequences

| | | SEQ |
|---|---|---|
| Protein | Sequence | ID NO |
| GARP | AQHQDKVPCKMVDKKVSCQVLGLLQVPSVLPPDTETLDLSGNQLRSILA | 41 |
| | SPLGFYTALRHLDLSTNEISFLQPGAFQALTHLEHLSLAHNRLAMATALS | |
| | AGGLGPLPRVTSLDLSGNSLYSGLLERLLGEAPSLHTLSLAENSLTRLTR | |
| | HTFRDMPALEQLDLHSNVLMDIEDGAFEGLPRLTHLNLSRNSLTCISDFS | |
| | LQQLRVLDLSCNSIEAFQTASQPQAEFQLTWLDLRENKLLHFPDLAALP | |
| | RLIYLNLSNNLIRLPTGPPQDSKGIHAPSEGWSALPLSAPSGNASGRPLS | |
| | QLLNLDLSYNEIELIPDSFLEHLTSLCFLNLSRNCLRTFEARRLGSLPCLM | |
| | LLDLSHNALETLELGARALGSLRTLLLQGNALRDLPPYTFANLASLQRLN | |
| | LQGNRVSPCGGPDEPGPSGCVAFSGITSLRSLSLVDNEIELLRAGAFLH | |
| | TPLTELDLSSNPGLEVATGALGGLEASLEVLALQGNGLMVLQVDLPCFIC | |
| | LKRLNLAENRLSHLPAWTQAVSLEVLDLRNNSFSLLPGSAMGGLETSLR | |
| | RLYLQGNPLSCCGNGWLAAQLHQGRVDVDATQDLICRFSSQEEVSLSH | |
| | VRPEDCEKGGLKNINLIIILTFILVSAILLTTLAACCCVRRQKFNQQYKA | |
| sGARP | AQHQDKVPCKMVDKKVSCQVLGLLQVPSVLPPDTETLDLSGNQLRSILA | 42 |
| | SPLGFYTALRHLDLSTNEISFLQPGAFQALTHLEHLSLAHNRLAMATALS | |
| | AGGLGPLPRVTSLDLSGNSLYSGLLERLLGEAPSLHTLSLAENSLTRLTR | |
| | HTFRDMPALEQLDLHSNVLMDIEDGAFEGLPRLTHLNLSRNSLTCISDFS | |
| | LQQLRVLDLSCNSIEAFQTASQPQAEFQLTWLDLRENKLLHFPDLAALP | |
| | RLIYLNLSNNLIRLPTGPPQDSKGIHAPSEGWSALPLSAPSGNASGRPLS | |
| | QLLNLDLSYNEIELIPDSFLEHLTSLCFLNLSRNCLRTFEARRLGSLPCLM | |
| | LLDLSHNALETLELGARALGSLRTLLLQGNALRDLPPYTFANLASLQRLN | |
| | LQGNRVSPCGGPDEPGPSGCVAFSGITSLRSLSLVDNEIELLRAGAFLH | |
| | TPLTELDLSSNPGLEVATGALGGLEASLEVLALQGNGLMVLQVDLPCFIC | |
| | LKRLNLAENRLSHLPAWTQAVSLEVLDLRNNSFSLLPGSAMGGLETSLR | |
| | RLYLQGNPLSCCGNGWLAAQLHQGRVDVDATQDLICRFSSQEEVSLSH | |
| | VRPEDCEKGGLKNIN | |
| LRRC33 (also known | MELLPLWLCLGFHFLTVGWRNRSGTATAASQGVCKLVGGAADCRGQ | 72 |
| as NRROS; Uniprot | SLASVPSSLPPHARMLTLDANPLKTLWNHSLQPYPLLESLSLHSCHLERI | |
| Accession No. | SRGAFQEQGHLRSLVLGDNCLSENYEETAAALHALPGLRRLDLSGNAL | |
| Q86YC3) | TEDMAALMLQNLSSLRSVSLAGNTIMRLDDSVFEGLERLRELDLQRNYI | |
| | FEIEGGAFDGLAELRHLNLAFNNLPCIVDFGLTRLRVLNVSYNVLEWFLA | |
| | TGGEAAFELETLDLSHNQLLFFPLLPQYSKLRTLLLRDNNMGFYRDLYN | |
| | TSSPREMVAQFLLVDGNVTNITTVSLWEEFSSSDLADLRFLDMSQNQF | |
| | QYLPDGFLRKMPSLSHLNLHQNCLMTLHIREHEPPGALTELDLSHNQLS | |
| | ELHLAPGLASCLGSLRLFNLSSNQLLGVPPGLFANARNITTLDMSHNQIS | |
| | LCPLPAASDRVGPPSCVDFRNMASLRSLSLEGCGLGALPDCPFQGTSL | |
| | TYLDLSSNWGVLNGSLAPLQDVAPMLQVLSLRNMGLHSSFMALDFSGF | |
| | GNLRDLDLSGNCLTTFPRFGGSLALETLDLRRNSLTALPQKAVSEQLSR | |
| | GLRTIYLSQNPYDCCGVDGWGALQHGQTVADWAMVTCNLSSKIIRVTE | |
| | LPGGVPRDCKWERLDLGLLYLVLILPSCLTLLVACTVIVLTFKKPLLQVIK | |
| | SRCHWSSVY | |
| | *Native signal peptide is depicted in bold font. | |
| soluble LRRC33 | MDMRVPAQLLGLLLLWFSGVLGWRNRSGTATAASQGVCKLVGGAAD | 73 |
| (sLRRC33) | CRGQSLASVPSSLPPHARMLTLDANPLKTLWNHSLQPYPLLESLSLHSC | |
| | HLERISRGAFQEQGHLRSLVLGDNCLSENYEETAAALHALPGLRRLDLS | |
| | GNALTEDMAALMLQNLSSLRSVSLAGNTIMRLDDSVFEGLERLRELDLQ | |
| | RNYIFEIEGGAFDGLAELRHLNLAFNNLPCIVDFGLTRLRVLNVSYNVLE | |
| | WFLATGGEAAFELETLDLSHNQLLFFPLLPQYSKLRTLLLRDNNMGFYR | |
| | DLYNTSSPREMVAQFLLVDGNVTNITTVSLWEEFSSSDLADLRFLDMSQ | |
| | NQFQYLPDGFLRKMPSLSHLNLHQNCLMTLHIREHEPPGALTELDLSHN | |
| | QLSELHLAPGLASCLGSLRLFNLSSNQLLGVPPGLFANARNITTLDMSH | |
| | NQISLCPLPAASDRVGPPSCVDFRNMASLRSLSLEGCGLGALPDCPFQ | |
| | GTSLTYLDLSSNWGVLNGSLAPLQDVAPMLQVLSLRNMGLHSSFMALD | |
| | FSGFGNLRDLDLSGNCLTTFPRFGGSLALETLDLRRNSLTALPQKAVSE | |
| | QLSRGLRTIYLSQNPYDCCGVDGWGALQHGQTVADWAMVTCNLSSKII | |
| | RVTELPGGVPRDCKWERLDLGLHHHHHH | |
| | *Modified human kappa light chain signal peptide is depicted in | |
| | bold font. | |
| | **Histidine tag is underlined. | |
| Human LRRC33-GARP | MDMRVPAQLLGLLLLWFSGVLGWRNRSGTATAASQGVCKLVGGAAD | 74 |
| chimera | CRGQSLASVPSSLPPHARMLTLDANPLKTLWNHSLQPYPLLESLSLHSC | |
| | HLERISRGAFQEQGHLRSLVLGDNCLSENYEETAAALHALPGLRRLDLS | |
| | GNALTEDMAALMLQNLSSLRSVSLAGNTIMRLDDSVFEGLERLRELDLQ | |
| | RNYIFEIEGGAFDGLAELRHLNLAFNNLPCIVDFGLTRLRVLNVSYNVLE | |
| | WFLATGGEAAFELETLDLSHNQLLFFPLLPQYSKLRTLLLRDNNMGFYR | |
| | DLYNTSSPREMVAQFLLVDGNVTNITTVSLWEEFSSSDLADLRFLDMSQ | |
| | NQFQYLPDGFLRKMPSLSHLNLHQNCLMTLHIREHEPPGALTELDLSHN | |
| | QLSELHLAPGLASCLGSLRLFNLSSNQLLGVPPGLFANARNITTLDMSH | |
| | NQISLCPLPAASDRVGPPSCVDFRNMASLRSLSLEGCGLGALPDCPFQ | |
| | GTSLTYLDLSSNWGVLNGSLAPLQDVAPMLQVLSLRNMGLHSSFMALD | |
| | FSGFGNLRDLDLSGNCLTTFPRFGGSLALETLDLRRNSLTALPQKAVSE | |
| | QLSRGLRTIYLSQNPYDCCGVDGWGALQHGQTVADWAMVTCNLSSKII | |
| | RVTELPGGVPRDCKWERLDLGLLIILTFILVSAILLTTLAACCCVRRQKFN | |
| | QQYKA | |
| | *Modified human kappa light chain signal peptide is depicted in | |
| | bold font. | |
| | **LRRC33 ectodomain is underlined. | |
| | #GARP transmembrane domain is italicized. | |
| | ##GARP intracellular tail is double underlined. | |

### Pharmaceutical Compositions and Formulations

The disclosure further provides pharmaceutical compositions used as a medicament suitable for administration in human and non-human subjects. One or more high-affinity, context-independent antibodies encompassed by the disclosure can be formulated or admixed with a pharmaceutically acceptable carrier (excipient), including, for example, a buffer, to form a pharmaceutical composition. Such formulations may be used for the treatment of a disease or disorder that involves TGFβ signaling. In certain embodiments, such formulations may be used for immuno-oncology applications.

The pharmaceutical compositions of the disclosure may be administered to patients for alleviating a TGFβ-related indication (e.g., fibrosis, immune disorders, and/or cancer). “Acceptable” means that the carrier is compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. In one example, a pharmaceutical composition described herein contains more than one antibody that specifically binds a GARP-proTGFβ1 complex, a LTBP1-proTGFβ1 complex, a LTBP3-proTGFβ1 complex, and a LRRC33-proTGFβ1 complex where the antibodies recognize different epitopes/residues of the complex.

The pharmaceutical compositions to be used in the present methods can comprise pharmaceutically acceptable carriers, excipients, or stabilizers in the form of lyophilized formulations or aqueous solutions (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONIC® or polyethylene glycol (PEG). Pharmaceutically acceptable excipients are further described herein.

The disclosure also includes pharmaceutical compositions that comprise an antibody or fragment thereof according to the present disclosure, and a pharmaceutically acceptable excipient.

Thus, the antibody or a molecule comprising an antigen-binding fragment of such antibody can be formulated into a pharmaceutical composition suitable for human administration.

The pharmaceutical formulation may include one or more excipients. In some embodiments, excipient(s) may be selected from the list provided in the following: https://www.accessdata.fda.gov/scripts/cder/ig/index.Cfm?event=browseByLetter.page&Letter=A

The pharmaceutical composition is typically formulated to a final concentration of the active biologic (e.g., monoclonal antibody, engineered binding molecule comprising an antigen-binding fragment, etc.) to be between about 20 mg/mL and about 200 mg/mL. For example, the final concentration (wt/vol) of the formulations may range between about 20-200, 20-180, 20-160, 20-150, 20-120, 20-100, 20-80, 20-70, 20-60, 20-50, 20-40, 30-200, 30-180, 30-160, 30-150, 30-120, 30-100, 30-80, 30-70, 30-60, 30-50, 30-40, 40-200, 40-180, 40-160, 40-150, 40-120, 40-100, 40-80, 40-70, 40-60, 40-50, 50-200, 50-180, 50-160, 50-150, 50-120, 50-100, 50-80, 50-70, 50-60, 60-200, 60-180, 60-160, 60-150, 60-120, 60-100, 60-80, 60-70, 70-200, 70-180, 70-160, 70-150, 70-120, 70-100, 70-80 mg/mL. In some embodiments, the final concentration of the biologic in the formulation is about 10, 15, 20, 25, 30, 35, 40, 45, 50, 55, 60, 65, 70, 75, 80, 85, 90, 95, 100, 110, 120, 130, 140, 150, 160, 170, 180, 190, or 200 mg/mL.

The pharmaceutical compositions of the present disclosure are preferably formulated with suitable buffers. Suitable buffers include but are not limited to: phosphate buffer, citric buffer, and histidine buffer.

The final pH of the formulation is typically between pH 5.0 and 8.0. For example, the pH of the pharmaceutical composition may be about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7 or 7.8.

The pharmaceutical composition of the present disclosure may comprise a surfactant, such as nonionic detergent, approved for the use in pharmaceutical formulations. Such surfactants include, for example, polysorbates, such as Polysorbate 20 (Tween™-20), Polysorbate 80 (Tween-80) and NP-40.

The pharmaceutical composition of the present disclosure may comprise a stabilizer. For liquid-protein preparations, stability can be enhanced by selection of pH-buffering salts, and often amino acids can also be used. It is often interactions at the liquid/air interface or liquid/solid interface (with the packaging) that lead to aggregation following adsorption and unfolding of the protein. Suitable stabilizers include but are not limited to: sucrose, maltose, sorbitol, as well as certain amino acids such as histidine, glycine, methionine and arginine.

The pharmaceutical composition of the present disclosure may contain one or any combinations of the following excipients: Sodium Phosphate, Arginine, Sucrose, Sodium Chloride, Tromethamine, Mannitol, Benzyl Alcohol, Histidine, Sucrose, Polysorbate 80, Sodium Citrate, Glycine, Polysorbate 20, Trehalose, Poloxamer 188, Methionine, Trehalose, Hyaluronidase, Sodium Succinate, Potassium Phosphate, Disodium Edetate, Sodium Chloride, Potassium Chloride, Maltose, Histidine Acetate, Sorbitol, Pentetic Acid, Human Serum Albumin, Pentetic Acid.

In some embodiments, the pharmaceutical composition of the present disclosure may contain a preservative.

The pharmaceutical composition of the present disclosure is typically presented as a liquid or a lyophilized form. Typically, the products can be presented in vial (e.g., glass vial). Products available in syringes, pens, or autoinjectors may be presented as pre-filled liquids in these container/closure systems.

In some examples, the pharmaceutical composition described herein comprises liposomes containing an antibody that specifically binds a GARP-proTGFβ1 complex, a LTBP1-proTGFβ1 complex, a LTBP3-proTGFβ1 complex, and a LRRC33-proTGFβ1 complex, which can be prepared by any suitable method, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, liposomes with targeting properties are selected to preferentially deliver or localize the pharmaceutical composition to certain tissues or cell types. For example, certain nanoparticle-based carriers with bone marrow-targeting properties may be employed, e.g., lipid-based nanoparticles or liposomes. See, for example, Sou (2012) “Advanced drug carriers targeting bone marrow”, ResearchGate publication 232725109.

In some embodiments, pharmaceutical compositions of the disclosure may comprise or may be used in conjunction with an adjuvant. It is contemplated that certain adjuvant can boost the subject's immune responses to, for example, tumor antigens, and facilitate T effector function, DC differentiation from monocytes, enhanced antigen uptake and presentation by APCs, etc. Suitable adjuvants include but are not limited to retinoic acid-based adjuvants and derivatives thereof, oil-in-water emulsion-based adjuvants, such as MF59 and other squalene-containing adjuvants, Toll-like receptor (TRL) ligands (e.g., CpGs), α-tocopherol (vitamin E) and derivatives thereof.

The antibodies described herein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid^{T}M Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an antibody of the disclosure with Intralipid™ or the components thereof (soybean oil, egg phospholipids, glycerol and water).

### Kits for Use in Detecting, Monitoring or Alleviating a TGFβ-Related Indication

The present disclosure also provides kits for use in treating, diagnosing, or predicting response in a subject having a diseases/disorders associated with a TGFβ-related indication. Such kits can include one or more containers comprising an antibody, or antigen binding portion thereof, that specifically binds to TGFβ (e.g., TGFβ1 or latent TGFβ1), phosphorylated SMAD (e.g., phosphorylated SMAD2), or any one of the surface markers described herein for identifying immune cells such as MDSCs (e.g., mMDSCs and/or gMDSCs) or CD8+ T cells.

In some embodiments, the kit can comprise instructions for use in accordance with any of the methods described herein. The included instructions can comprise a description of using the antibody, or antigen binding portion thereof, that specifically binds TGFβ (e.g., TGFβ1 or latent TGFβ1), phosphorylated SMAD (e.g., phosphorylated SMAD2), or any one of the surface markers described herein for identifying immune cells such as MDSCs (e.g., mMDSCs and/or gMDSCs) or CD8+ T cells to treat, diagnose, or predict response in a subject having a target disease described herein. In some embodiments, the kit may comprise a description of selecting an individual suitable for treatment based on identifying whether that individual has the target disease or is likely to achieve clinical efficacy following treatment with a particular regimen, e.g., a treatment comprising a TGFβ1 inhibitor, e.g., Ab6, alone or in conjunction with a checkpoint inhibitor therapy or a chemotherapy. In still other embodiments, the instructions comprise a description for determining target engagement or therapeutic efficacy in an individual treated with a TGFβ1 inhibitor, e.g., Ab6.

In some embodiments, the instructions may include information such as use of the antibodies or antigen-binding fragments binding to biomarkers described herein, e.g., TGFβ (e.g., TGFβ1 or latent TGFβ1), phosphorylated SMAD (e.g., phosphorylated SMAD2), or any one of the surface markers described herein for identifying immune cells such as MDSCs (e.g., mMDSCs and/or gMDSCs) or CD8+ T cells, as part of a treatment regimen. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, diagnosing, or predicting response for a disease or disorder associated with a TGFβ-related indication. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the disclosure provides articles of manufacture comprising contents of the kits described above.

### Process of Screening, Identification and Manufacture of Preferred Isoform-Specific Inhibitors of TGFβ1

The disclosure encompasses screening/selection methods, production methods and manufacture processes of antibodies or fragments thereof capable of binding each of: a GARP-proTGFβ1 complex, a LTBP1-proTGFβ1 complex, a LTBP3-proTGFβ1 complex, and a LRRC33-proTGFβ1 complex with equivalent affinities, and pharmaceutical compositions and related kits comprising the same. In some embodiments, for screening purposes, at least one of the LTBP1-proTGFβ1 and LTBP3-proTGFβ1 complexes and at least one of the GARP-proTGFβ1 and LRRC33-proTGFβ1 complexes are included. Antibodies or fragments thereof identified in the screening process are preferably further tested to confirm its ability to bind each of the LLCs of interest with high affinity.

Numerous methods may be used for obtaining antibodies, or antigen binding fragments thereof, of the disclosure. For example, antibodies can be produced using recombinant DNA methods. Monoclonal antibodies may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET® or BIACORE) analysis, to identify one or more hybridomas that produce an antibody that specifically binds to a specified antigen. Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al., (1991) Nature, 352: 624-628; Marks et al., (1991) J. Mol. Biol., 222: 581-597; WO 92/18619; WO 91/17271; WO 92/20791; WO 92/15679; WO 93/01288; WO 92/01047; WO 92/09690; and WO 90/02809.

In addition to the use of display libraries, the specified antigen (e.g., presenting molecule-TGFβ1 complexes) can be used to immunize a non-human host, e.g., rabbit, guinea pig, rat, mouse, hamster, sheep, goat, chicken, camelid, as well as non-mammalian hosts such as shark. In one embodiment, the non-human animal is a mouse.

Immunization of a non-human host may be carried out with the use of a purified recombinant protein complex as an immunogen, such as proTGFβ1 with or without a presenting molecule (or fragment thereof) associated thereto. These include, but are not limited to: LTBP1-proTGFβ1, LTBP3-proTGFβ1, GARP-proTGFβ1 and LRRC33-proTGFβ1. The associated presenting molecule need not be full length counterpart but preferably includes the two cysteine residues that form covalent bonds with the proTGFβ1 dimer complex.

Alternatively, immunization of a non-human host may be carried out with the use of a cell-based antigen. The term cell-based antigen refers to cells (e.g., heterologous cells) expressing the proTGFβ1 protein complex. This may be achieved by overexpression of proTGFβ1, optionally with co-expression of a presenting molecule. In some embodiments, endogenous counterpart(s) may be utilized as cell-based antigen. Cell-surface expression of the proteins that form the proTGFβ1-containing protein complex may be confirmed by well-known methods such as FACS. Upon immunization of the host with such cells (a cell-based antigen), immune responses to the antigen are elicited in the host, allowing antibody production and subsequent screening. In some embodiments, suitable knockout animals are used to facilitate stronger immune responses to the antigen. Alternatively, structural differences among different species may be sufficient to trigger antibody production in the host.

In another embodiment, a monoclonal antibody is obtained from the non-human animal, and then modified, e.g., chimeric, using suitable recombinant DNA techniques. Avariety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

For additional antibody production techniques, see Antibodies: A Laboratory Manual, eds. Harlow et al., Cold Spring Harbor Laboratory, 1988. The present disclosure is not necessarily limited to any particular source, method of production, or other special characteristics of an antibody.

Some aspects of the present disclosure relate to host cells transformed with a polynucleotide or vector. Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies, antigen binding fragments or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments. Large-scale production of the antibody or antibody fragments (for example, about 250 L or greater, e.g., 1000L, 2000L, 3000L, 4000L or greater) is suitable for commercial-scale manufacture of pharmaceutical compositions comprising the antibody and is typically carried out in a culture system, such as a suspension cell culture. Such culture may be a eukaryotic cell culture, wherein optionally the eukaryotic cell culture is a mammalian cell culture, plant cell culture, or an insect cell culture. In some embodiments, the mammalian cell culture comprises a CHO cell, MDCK cell, NS0 cell, Sp2/0 cell, BHK cell, Murine C127 cell, Vero cell, HEK293 cell, HT-1080 cell, or PER.C6 cell.

The transformed host cells can be grown in fermenters and cultured using any suitable techniques to achieve optimal cell growth. Once expressed, the whole antibodies, their dimers, individual light and heavy chains, other immunoglobulin forms, or antigen binding fragments, can be purified according to standard procedures of the art, including ammonium sulfate precipitation, affinity columns, column chromatography, gel electrophoresis and the like; see, Scopes, Protein Purification, Springer Verlag, N.Y. (1982). The antibody or antigen binding fragments can then be isolated from the growth medium, cellular lysates, or cellular membrane fractions. The isolation and purification of the, e.g., microbially expressed antibodies or antigen binding fragments may be by any conventional means such as, for example, preparative chromatographic separations and immunological separations such as those involving the use of monoclonal or polyclonal antibodies directed, e.g., against the constant region of the antibody.

Aspects of the disclosure relate to a hybridoma, which provides an indefinitely prolonged source of monoclonal antibodies. As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. In some embodiments, heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment may be undertaken by culturing a modified recombinant host under culture conditions appropriate for the growth of the host cells and the expression of the coding sequences. The antibodies or antigen binding fragments may be recovered by isolating them from the culture. The expression systems may be designed to include signal peptides so that the resulting antibodies are secreted into the medium; however, intracellular production is also possible.

The disclosure also includes a polynucleotide encoding at least a variable region of an immunoglobulin chain of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast orthe CMV-promoter, SV40-promoter, RSV-promoter (Rous sarcoma virus), CMV-enhancer, SV40-enhancer or a globin intron in mammalian and other animal cells.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and have been described previously. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains or only one. Likewise, polynucleotides may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen binding fragment; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen binding fragment). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

The screening methods may include a step of evaluating or confirming desired activities of the antibody or fragment thereof. In some embodiments, the step comprises selecting for the ability to inhibit target function, e.g., inhibition of release of mature/soluble growth factor (e.g., TGFβ1) from a latent complex. In certain embodiments, such step comprises a cell-based potency assay, in which inhibitory activities of test antibody or antibodies are assayed by measuring the level of growth factor released in the medium (e.g., assay solution) upon activation, when proTGFβ complex is expressed on cell surface. The level of growth factor released into the medium/solution can be assayed by, for example, measuring TGFβ activities. Non-limiting examples of useful cell-based potency assays are described in PCT/US2019/041373, e.g., at Example 2.

In some embodiments, the step of screening desirable antibodies or fragments comprises selecting for antibodies or fragments thereof that promote internalization and subsequent removal of antibody-antigen complexes from the cell surface. In some embodiments, the step comprises selecting for antibodies or fragments thereof that induce ADCC. In some embodiments, the step comprises selecting for antibodies or fragments thereof that accumulate to a desired site(s) in vivo (e.g., cell type, tissue or organ). In some embodiments, the step comprises selecting for antibodies or fragments thereof with the ability to cross the blood brain barrier. The methods may optionally include a step of optimizing one or more antibodies or fragments thereof to provide variant counterparts that possess desirable profiles, as determined by criteria such as stability, binding affinity, functionality (e.g., inhibitory activities, Fc function, etc.), immunogenicity, pH sensitivity and developability (e.g., high solubility, low self-association, etc.).

The process for making a composition comprising an antibody or a fragment according to the disclosure may include optimization of an antibody or antibodies that are identified to possess desirable binding and functional (e.g., inhibitory) properties. Optimization may comprise affinity maturation of an antibody or fragment thereof. Further optimization steps may be carried out to provide physicochemical properties that are advantageous for therapeutic compositions. Such steps may include, but are not limited to, mutagenesis or engineering to provide improved solubility, lack of self-aggregation, stability, pH sensitivity, Fc function, and so on. The resulting optimized antibody is preferably a fully human antibody or humanized antibody suitable for human administration.

Manufacture process for a pharmaceutical composition comprising such an antibody or fragment thereof may comprise the steps of purification, formulation, sterile filtration, packaging, etc. Certain steps such as sterile filtration, for example, are performed in accordance with the guidelines set forth by relevant regulatory agencies, such as the FDA. Such compositions may be made available in a form of single-use containers, such as pre-filled syringes, or multi-dosage containers, such as vials.

### Modifications

Antibodies, or antigen binding portions thereof, of the disclosure may be modified with a detectable label or detectable moiety, including, but not limited to, an enzyme, prosthetic group, fluorescent material, luminescent material, bioluminescent material, radioactive material, positron emitting metal, nonradioactive paramagnetic metal ion, and affinity label for detection and isolation of a GARP-proTGFβ1 complex, a LTBP1-proTGFβ1 complex, a LTBP3-proTGFβ1 complex, and/or a LRRC33-proTGFβ1 complex. The detectable substance or moiety may be coupled or conjugated either directly to the polypeptides of the disclosure or indirectly, through an intermediate (such as, for example, a linker (e.g., a cleavable linker)) using suitable techniques. Non-limiting examples of suitable enzymes include horseradish peroxidase, alkaline phosphatase, β-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (131I, 125I, 123I, 121I), carbon (14C), sulfur (35S), tritium (3H), indium (115mIn, 113mIn, 112In, 111In), and technetium (99Tc, 99mTc), thallium (201Ti), gallium (68Ga, 67Ga), palladium (103Pd), molybdenum (99Mo), xenon (133Xe), fluorine (18F), 153Sm, Lu (177Lu), 159Gd, 149Pm, 140La, 175Yb, 166Ho, 90Y, 47Sc, 86R, 188Re, 142Pr, 105Rh, 97Ru, 68Ge, 57Co, 65Zn, 85Sr, 32P, 153Gd, 169Yb, 51Cr, 54Mn, 75Se, Zirconium (⁸⁹Zr) and tin (113Sn, 117Sn). In some embodiments, the radio label may be selected from the group consisting of: ¹¹C, ¹³N, ¹⁵O, ⁶⁸Ga, ¹⁷⁷Lu, ¹⁸F and ⁸⁹Zr. In some embodiments, useful labels are positron-emitting isotopes, which may be detected by positron-emission tomography. The detectable substance may be coupled or conjugated either directly to the antibodies of the disclosure that bind specifically to a GARP-proTGFβ1 complex, a LTBP1-proTGFβ1 complex, a LTBP3-proTGFβ1 complex, and/or a LRRC33-proTGFβ1 complex, or indirectly, through an intermediate (such as, for example, a linker) using suitable techniques. Any of the antibodies provided herein that are conjugated to a detectable substance may be used for any suitable diagnostic assays, such as those described herein.

In addition, antibodies, or antigen binding portions thereof, of the disclosure may also be modified with a drug. The drug may be coupled or conjugated either directly to the polypeptides of the disclosure, or indirectly, through an intermediate (such as, for example, a linker (e.g., a cleavable linker)) using suitable techniques.

### Targeting Agents

In some embodiments methods of the present disclosure comprise the use of one or more targeting agents to target an antibody, or antigen binding portion thereof, as disclosed herein, to a particular site in a subject for purposes of modulating mature TGFβ release from a GARP-proTGFβ1 complex, a LTBP1-proTGFβ1 complex, a LTBP3-proTGFβ1 complex, and/or a LRRC33-proTGFβ1 complex. For example, LTBP1-proTGFβ1 and LTBP3-proTGFβ1 complexes are typically localized to extracellular matrix. Thus, in some embodiments, antibodies disclosed herein can be conjugated to extracellular matrix targeting agents for purposes of localizing the antibodies to sites where LTBP-associated TGFβ1 complexes reside. In such embodiments, selective targeting of antibodies leads to selective modulation of LTBP1-proTGFβ1 and LTBP3-proTGFβ1 complexes. In some embodiments, extracellular matrix targeting agents include heparin binding agents, matrix metalloproteinase binding agents, lysyl oxidase binding domains, fibrillin-binding agents, hyaluronic acid binding agents, and others.

Similarly, GARP-proTGFβ1 and LRRC33-proTGFβ1 complexes are typically localized and anchored to the surface of cells. The former is expressed on activated FOXP3+ regulatory T cells (Tregs), while the latter is expressed on certain myeloid cells and some cancer cells such as AML. Thus, in some embodiments, antibodies disclosed herein can be conjugated to immune cell (e.g., Treg cell, activated macrophages, etc.) binding agents for purposes of localizing antibodies to sites where these cell-associated proTGFβ1 complexes reside. In such embodiments, selective targeting of antibodies leads to selective inhibition of cell associated-proTGFβ1 complexes (e.g., selective inhibition of the release of mature TGFβ1 for purposes of immune modulation, e.g., in the treatment of cancer). In such embodiments, immune cell targeting agents may include, for example, CCL22 and CXCL12 proteins or fragments thereof.

In some embodiments, bispecific antibodies may be used having a first portion that selectively binds a proTGFβ1 complex and a second portion that selectively binds a component of a target site, e.g., a component of the ECM (e.g., fibrillin) or a component of a Treg cell (e.g., CTLA-4).

As further detailed herein, the present disclosure contemplates that isoform-selective TGFβ1 inhibitors, such as those described herein, may be used for promoting or restoring hematopoiesis in the bone marrow. Accordingly, in some embodiments, a composition comprising such an inhibitor (e.g., high-affinity, isoform-selective inhibitor of TGFβ1) may be targeted to the bone marrow. One mode of achieving bone marrow targeting is the use of certain carriers that preferentially target the bone marrow localization or accumulation. For example, certain nanoparticle-based carriers with bone marrow-targeting properties may be employed, e.g., lipid-based nanoparticles or liposomes. See, for example, Sou (2012) “Advanced drug carriers targeting bone marrow”, ResearchGate publication 232725109.

In some embodiments, targeting agents include immune-potentiators, such as adjuvants comprising squalene and/or α-tocopherol and adjuvants comprising a TLR ligand/agonist (such as TLR3 ligands/agonists). For example, squalene-containing adjuvant may preferentially target certain immune cells such as monocytes, macrophages and antigen-presenting cells to potentiate priming, antigen processing and/or immune cell differentiation to boost host immunity. In some embodiments, such adjuvant may stimulate host immune responses to neo-epitopes for T cell activation.

### Therapeutic Targets and In Vivo Mechanisms of Action

Accordingly, the TGFβ inhibitors (e.g., high-affinity, isoform-selective TGFβ1 inhibitors) disclosed herein may be used to inhibit TGFβ1 in any suitable biological systems, such as in vitro, ex vivo and/or in vivo systems. Related methods may comprise contacting a biological system with the TGFβ1 inhibitor. The biological system may be an assay system, a biological sample, a cell culture, and so on. In some cases, these methods include modifying the level of free growth factor in the biological system.

Accordingly, such pharmaceutical compositions and formulations may be used to target TGFβ-containing latent complexes accessible by the inhibitors in vivo. Thus, the antibody of the disclosure is aimed to target the following complexes in a disease site (e.g., TME) where it preemptively binds the latent complex thereby preventing the growth factor from being released: i) proTGFβ1 presented by GARP; ii) proTGFβ1 presented by LRRC33; iii) proTGFβ1 presented by LTBP1; and iv) proTGFβ1 presented by LTBP3. Typically, complexes (i) and (ii) above are present on cell surface because both GARP and LRRC33 are transmembrane proteins capable of anchoring or tethering latent proTGFβ1 on the extracellular face of the cell expressing LRRC33, whilst complexes (iii) and (iv) are components of the extracellular matrix. In this way, the inhibitors embodied herein do away with having to complete binding with endogenous high affinity receptors for exerting inhibitory effects. Moreover, targeting upstream of the ligand/receptor interaction may enable more durable effects since the window of target accessibility is longer and more localized to relevant tissues than conventional inhibitors that target transient, soluble growth factors only after it has been released from the latent complex. Thus, targeting the latent complex tethered to certain niches may facilitate improved target engagement in vivo, as compared to conventional neutralizing antibodies that must compete binding with endogenous receptors during its short half-life as a soluble (free) growth factor, e.g., ˜two minutes, once it is released from the latent complex.

A number of studies have shed light on the mechanisms of TGFβ1 activation. Three integrins, αVβ1, αVβ6, αVβ8, and αVβ1 have been demonstrated to be key activators of latent TGFβ1 (Reed, N. I., et al., Sci Transl Med, 2015. 7(288): p. 288ra79; Travis, M. A. and D. Sheppard, Annu Rev Immunol, 2014. 32: p. 51-82; Munger, J. S., et al., Cell, 1999. 96(3): p. 319-28; Sheppard. Cancer Metastasis Rev, 2005. 24(3): 395-402). αV integrins bind the RGD sequence present in TGFβ1 and TGFβ1 LAPs with high affinity (Dong, X., et al., Nat Struct Mol Biol, 2014. 21(12): p. 1091-6). Transgenic mice with a mutation in the TGFβ1 RGD site that prevents integrin binding, but not secretion, phenocopy the TGFβ1−/− mouse (Yang, Z., et al., J Cell Biol, 2007. 176(6): p. 787-93). Mice that lack both β36 and β8 integrins recapitulate all essential phenotypes of TGFβ1 and TGFβ3 knockout mice, including multiorgan inflammation and cleft palate, confirming the essential role of these two integrins for TGFβ1 activation in development and homeostasis (Aluwihare, P., et al., J Cell Sci, 2009. 122(Pt 2): p. 227-32). Key for integrin-dependent activation of latent TGFβ1 is the covalent tether to presenting molecules; disruption of the disulfide bonds between GARP and TGFβ1 LAP by mutagenesis does not impair complex formation, but completely abolishes TGFβ1 activation by αVβ6 (Wang, R., et al., Mol Biol Cell, 2012. 23(6): p. 1129-39). The recent structure study of latent TGFβ1 illuminates how integrins enable release of active TGFβ1 from the latent complex: the covalent link of latent TGFβ1 to its presenting molecule anchors latent TGFβ1, either to the ECM through LTBPs, or to the cytoskeleton through GARP or LRRC33. Integrin binding to the RGD sequence results in a force-dependent change in the structure of LAP, allowing active TGFβ1 to be released and bind nearby receptors (Shi, M., et al., Nature, 2011. 474(7351): p. 343-9). The importance of integrin-dependent TGFβ1 activation in disease has also been well validated. A small molecule inhibitor of αVβ1 protects against bleomycin-induced lung fibrosis and carbon tetrachloride-induced liver fibrosis (Reed, N. I., et al., Sci Transl Med, 2015. 7(288): p. 288ra79), and αVβ6 blockade with an antibody or loss of integrin 136 expression suppresses bleomycin-induced lung fibrosis and radiation-induced fibrosis (Munger, J. S., et al., Cell, 1999. 96(3): p. 319-28); Horan, G. S., et al., Am J Respir Crit Care Med, 2008. 177(1): p. 56-65).

In addition to integrins, other mechanisms of TGFβ1 activation have been implicated, including thrombospondin-1 and activation by proteases such as Plasmin, matrix metalloproteinases (MMPs, e.g., MMP2, MMP9 and MMP12), cathepsin D, kallikrein, thrombin, and the ADAMs family of zinc proteases (e.g., ADAM10, ADAM12 and ADAM17). Knockout of thrombospondin-1 recapitulates some aspects of the TGFβ1−/− phenotype in some tissues, but is not protective in bleomycin-induced lung fibrosis, known to be TGFβ-dependent (Ezzie, M. E., et al., Am J Respir Cell Mol Biol, 2011. 44(4): p. 556-61). Additionally, knockout of candidate proteases did not result in a TGFβ1 phenotype (Worthington, J. J., J. E. Klementowicz, and M. A. Travis, Trends Biochem Sci, 2011. 36(1): p. 47-54). This could be explained by redundancies or by these mechanisms being critical in specific diseases rather than development and homeostasis.

Thus, the TGFβ inhibitors (e.g., high-affinity, isoform-specific inhibitors of TGFβ1) described herein include inhibitors that work by preventing the step of TGFβ1 activation. In some embodiments, such inhibitors can inhibit integrin-dependent (e.g., mechanical or force-driven) activation of TGFβ1. In some embodiments, such inhibitors can inhibit protease-dependent or protease-induced activation of TGFβ1. The latter includes inhibitors that inhibit the TGFβ1 activation step in an integrin-independent manner. In some embodiments, such inhibitors can inhibit TGFβ1 activation irrespective of the mode of activation, e.g., inhibit both integrin-dependent activation and protease-dependent activation of TGFβ1. Non-limiting examples of proteases which may activate TGFβ1 include serine proteases, such as Kallikreins, Chemotrypsin, Trypsin, Elastases, Plasmin, thrombin, as well as zinc metalloproteases (MMP family) such as MMP-2, MMP-9 and MMP-13. Kallikreins include plasma-Kallikreins and tissue Kallikreins, such as KLK1, KLK2, KLK3, KLK4, KLK5, KLK6, KLK7, KLK8, KLK9, KLK10, KLK11, KLK12, KLK13, KLK14 and KLK15. Data previously presented in PCT/US2019/041373 demonstrate examples of an isoform-specific TGFβ1 inhibitors, capable of inhibiting Kallikrein-dependent activation of TGFβ1 in vitro. In some embodiments, inhibitors of the present disclosure prevent release or dissociation of active (mature) TGFβ1 growth factor from the latent complex. In some embodiments, such inhibitors may work by stabilizing the inactive (e.g., latent) conformation of the complex. Applicant's previous data further demonstrated that a high-affinity, context-independent TGFβ1 inhibitor (e.g, Ab6) can also inhibit Plasmin-dependent TGFβ1 activation. Surprisingly, however, a context-biased TGFβ1 inhibitor (Ab3) was less effective to inhibit this process. Both Ab3 and Ab6 have similar affinities for matrix-associated proTGFβ1 complexes. However, Ab3 has a significantly weaker binding affinity for cell-associated proTGFβ1 complexes. The relative difference between the two categories is more than 20-fold (“bias”). By comparison, Ab6 shows equivalent high affinities towards both categories of the antigen complexes. One possible explanation is that the observed functional difference may stem from the bias feature of Ab3. Another possible explanation is that it is mediated by differences in epitopes or binding regions.

The disclosure is particularly useful for therapeutic use for certain diseases that are associated with multiple biological roles of TGFβ signaling that are not limited to a single context of TGFβ function. In such situations, it may be beneficial to inhibit TGFβ1 effects across multiple contexts. Thus, the present disclosure provides methods for targeting and inhibiting TGFβ1 in an isoform-specific manner, rather than in a context-specific manner. Such agents may be referred to as “isoform-specific, context-independent” TGFβ1 modulators.

A body of evidence supports the notion that many diseases manifest complex perturbations of TGFβ signaling, which likely involve participation of heterogeneous cell types that confer different effects of TGFβ function, which are mediated by its interactions with so-called presenting molecules. At least four such presenting molecules have been identified, which can “present” TGFβ in various extracellular niches to enable its activation in response to local stimuli. In one category, TGFβ is deposited into the ECM in association with ECM-associated presenting molecules, such as LTBP1 and LTBP3, which mediate ECM-associated TGFβ activities. In another category, TGFβ is tethered onto the surface of immune cells, via presenting molecules such as GARP and LRRC33, which mediate certain immune function. These presenting molecules show differential expression, localization and/or function in different tissues and cell types, indicating that triggering events and outcome of TGFβ activation will vary, depending on the microenvironment. Based on the notion that many TGFβ effects may interact and contribute to disease progression, therapeutic agents that can antagonize multiple facets of TGFβ function may provide greater efficacy.

### Lrrc33-proTGFβ1 as Target

LRRC33 is expressed in selective cell types, in particular those of myeloid lineage, including monocytes and macrophages. Monocytes originated from progenitors in the bone marrow and circulate in the bloodstream and reach peripheral tissues. Circulating monocytes can then migrate into tissues where they become exposed to the local environment (e.g., tissue-specific, disease-associated, etc.) that includes a panel of various factors, such as cytokines and chemokines, triggering differentiation of monocytes into macrophages, dendritic cells, etc. These include, for example, alveolar macrophages in the lung, osteoclasts in bone marrow, microglia in the CNS, histiocytes in connective tissues, Kupffer cells in the liver, and brown adipose tissue macrophages in brown adipose tissues. In a solid tumor, infiltrated macrophages may be tumor-associated macrophages (TAMs), tumor-associated neutrophils (TANs), and myeloid-derived suppressor cells (MDSCs), etc. Such macrophages may activate and/or be associated with activated fibroblasts, such as carcinoma-associated (or cancer-associated) fibroblasts (CAFs) and/or the stroma. Thus, inhibitors of TGFβ1 activation described herein which inhibits release of mature TGFβ1 from LRRC33-containing complexes can target any of these cells expressing LRRC33-proTGFβ1 on cell surface. At a fibrotic microenvironment, LRRC33-expressing cells may include M2 macropahges, tissue resident macrophages, and/or MDSCs.

In some embodiments, the LRRC33-TGFβ1 complex is present at the outer surface of profibrotic (M2-like) macrophages. In some embodiments, the profibrotic (M2-like) macrophages are present in the fibrotic microenvironment. In some embodiments, targeting of the LRRC33-TGFβ1 complex at the outer surface of profibrotic (M2-like) macrophages provides a superior effect as compared to solely targeting LTBP1-TGFβ1 and/or LTBP1-TGFβ1 complexes. In some embodiments, M2-like macrophages, are further polarized into multiple subtypes with differential phenotypes, such as M2c and M2d TAM-like macrophages. In some embodiments, macrophages may become activated by various factors (e.g., growth factors, chemokines, cytokines and ECM-remodeling molecules) present in the tumor microenvironment, including but are not limited to TGFβ1, CCL2 (MCP-1), CCL22, SDF-1/CXCL12, M-CSF (CSF-1), IL-6, IL-8, IL-10, IL-11, CXCR4, VEGF, PDGF, prostaglandin-regulating agents such as arachidonic acid and cyclooxygenase-2 (COX-2), parathyroid hormone-related protein (PTHrP), RUNX2, HIF1α, and metalloproteinases. Exposures to one or more of such factors may further drive monocytes/macrophages into pro-tumor phenotypes. To give but one example, CCL2 and VEGF co-expression in tumors has been shown to be correlated with increased TAM and poor diagnosis. In turn, activated tumor-associated cells may also facilitate recruitment and/or differentiation of other cells into pro-tumor cells, e.g., CAFs, TANs, MDSCs, and the like. Stromal cells may also respond to macrophage activation and affect ECM remodeling, and ultimately vascularization, invasion, and metastasis. For example, CCL2 not only functions as a monocyte attractant but also promotes cell adhesion by upregulating MAC-1, which is a receptor for ICAM-1, expressed in activated endothelium. This may lead to CCL2-dependent arteriogenesis and cancer progression. Thus, TGFβ1 inhibitors described herein may be used in a method for inhibiting arteriogenesis by interfering with the CCL2 signaling axis.

A subset of myeloid cells express cell surface LRRC33, including M2-polarized macrophages and myeloid-derived suppressor cells (MDSCs), both of which have immunosuppressive phenotypes and are enriched at disease environments (e.g., TME and FME). Bone marrow-derived circulating monocytes do not appear to express cell surface LRRC33. The restrictive expression of LRRC33 makes this a particularly appealing therapeutic target. While a majority of studies available in the literature have focused on effector T cell biology (e.g., CD8+ cytotoxic cells) in cancer, increasing evidence (such as data presented in PCT/US2019/041373) points to important roles of suppressive myeloid cell populations in diseases. Importantly, the highly selective TGFβ1 inhibitory antibodies disclosed herein, are capable of targeting this arm of TGFβ1 function in vivo. More specifically, tumor-associated M2 macrophages and MDSCs express cell-surface LRRC33, with a strong correlation to disease progression. The high-affinity, TGFβ1-selective antibodies disclosed herein are capable of overcoming primary resistance to checkpoint blockade therapy (CBT) of tumors in multiple pharmacological models. Indeed, anti-tumor efficacy coincides with a significant decrease in tumor-associated macrophages and MDSC levels, suggesting that targeting this facet of TGFβ1 function may contribute to therapeutically beneficial effects. This is likely applicable to other disease where these immunosuppressive cells are enriched. A number of fibrotic conditions are also associated with elevated local frequencies of these cell populations. Thus, the high-affinity, TGFβ1-selective antibodies are expected to exert similar in vivo effects in such indications. Notably, the new findings provided herein identify LRRC33 as a novel cell surface marker for MDSCs in circulation, this, coupled with Applicant's previous findings that i) the degree of tumor burden correlates with tumor associated MDSCs, and that ii) tumor-associated MDSCs correlate with circulatory MDSCs, points to a new use for LRRC33 as a blood-based biomarker, e.g., indicative of immune suppression. As described herein, an immune-suppressed tumor may be particularly responsive to treatment comprising a TGFβ inhibitor (e.g., TGFβ1-selective inhibitor, e.g., Ab6) and optionally in combination with a checkpoint inhibitor therapy.

### TGFβ1-Related Indications

### General Features of TGFβ1-Related Indications

TGFβ1 inhibitors, such as isoform-selective inhibitors described herein, may be used to treat a wide variety of diseases, disorders and/or conditions that are associated with TGFβ1 dysregulation (i.e., “TGFβ1-related indications”) in human subjects. As used herein, “disease (disorder or condition) associated with TGFβ1 dysregulation” or “TGFβ1-related indication” means any disease, disorder and/or condition related to expression, activity and/or metabolism of a TGFβ1 or any disease, disorder and/or condition that may benefit from inhibition of the activity and/or levels TGFβ1. A plethora of evidence exists in literature pointing to the dysregulation of the TGFβ signaling pathway in pathologies such as cancer and fibrosis.

Based on the inventors' recognition that TGFβ1 appears to be the predominant disease-associated isoform, the present disclosure includes the use of an isoform-selective, context-independent TGFβ1 inhibitor in a method for treating a TGFβ1-related indication in a human subject. Such inhibitor is typically formulated into a pharmaceutical composition that further comprises a pharmaceutically acceptable excipient. Advantageously, the inhibitor targets both ECM-associated TGFβ1 and immune cell-associated TGFβ1 but does not target TGFβ2 or TGFβ3 in vivo. In some embodiments, the inhibitor inhibits the activation step of TGFβ1. The disease may be characterized by dysregulation or impairment in at least two of the following attributes: a) regulatory T cells (Treg); b) effector T cell (Teff) proliferation or function; c) myeloid cell proliferation or differentiation; d) monocyte recruitment or differentiation; e) macrophage function; f) epithelial-to-mesenchymal transition (EMT) and/or endothelial-to-mesenchymal transition (EndMT); g) gene expression in one or more of marker genes selected from the group consisting of: PAI-1, ACTA2, CCL2, Col1a1, Col3a1, FN-1, CTGF, and TGFB1; h) ECM components or function; i) fibroblast differentiation. A therapeutically effective amount of such inhibitor is administered to the subject suffering from or diagnosed with the disease.

In some embodiments, such therapeutic use incorporates the step of diagnosing and/or monitoring treatment response as detailed herein. For example, circulating MDSCs and/or circulating latent_TGFβ1 may be used as biomarker, in accordance with the present disclosure. Such therapeutic use may further include a step of selecting a suitable TGFβ inhibitor as therapy and/or selecting a patient or patient population likely to benefit from such therapy.

In some embodiments, a disease treated herein may involve dysregulation or impairment of ECM components or function comprises that show increased collagen I deposition. In some embodiments, the dysregulation of the ECM includes increased stiffness of the matrix. In some embodiments, the dysregulation of the ECM involves fibronectin and/or fibrillin.

In some embodiments, the dysregulation or impairment of fibroblast differentiation comprises increased myofibroblasts or myofibroblast-like cells. In some embodiments, the myofibroblasts or myofibroblast-like cells are cancer-associated fibroblasts (CAFs). In some embodiments, the CAFs are associated with a tumor stroma and may produce CCL2/MCP-1 and/or CXCL12/SDF-1. In some embodiments, the myofibroblasts or myofibroblast-like cells are localized to a fibrotic tissue.

In some embodiments, the dysregulation or impairment of regulatory T cells comprises increased Treg activity.

In some embodiments, the dysregulation or impairment of effector T cell (Teff) proliferation or function comprises suppressed CD4+/CD8+ cell proliferation.

In some embodiments, the dysregulation or impairment of myeloid cell proliferation or differentiation comprises increased proliferation of myeloid progenitor cells. The increased proliferation of myeloid cells may occur in a bone marrow,

In some embodiments, the dysregulation or impairment of monocyte differentiation comprises increased differentiation of bone marrow-derived and/or tissue resident monocytes into macrophages at a disease site, such as a fibrotic tissue and/or a solid tumor.

In some embodiments, the dysregulation or impairment of monocyte recruitment comprises increased bone marrow-derived monocyte recruitment into a disease site such as TME, leading to increased macrophage differentiation and M2 polarization, followed by increased TAMs.

In some embodiments, the dysregulation or impairment of macrophage function comprises increased polarization of the macrophages into M2 phenotypes.

In some embodiments, the dysregulation or impairment of myeloid cell proliferation or differentiation comprises an increased number of Tregs, MDSCs and/or TANs.

TGFβ-related indications may include conditions comprising an immune-excluded disease microenvironment, such as tumor or cancerous tissue that suppresses the body's normal defense mechanism/immunity in part by excluding effector immune cells (e.g., CD4+ and/or CD8+ T cells). In some embodiments, such immune-excluding conditions are associated with poor responsiveness to treatment (e.g., cancer therapy). Non-limiting examples of the cancer therapies, to which patients are poorly responsive, include but are not limited to: checkpoint inhibitor therapy, cancer vaccines, chemotherapy, and radiation therapy (such as a radiotherapeutic agent). Without intending to be bound by particular theory, it is contemplated that TGFβ inhibitors, such as those described herein, may help counter the tumor's ability to evade or exclude anti-cancer immunity by restoring immune cell access, e.g., T cell (e.g., CD8+ cells) and macrophage (e.g., F4/80+ cells, M1-polarized macrophages) access by promoting T cell expansion and/or infiltration into tumor.

Thus, TGFβ inhibition may overcome treatment resistance (e.g., immune checkpoint resistance, cancer vaccine resistance, CAR-T resistance, chemotherapy resistance, radiation therapy resistance (such as resistance to a radiotherapeutic agent), etc.) in immune-excluded disease environment (such as TME) by unblocking and restoring effector T cell access and cytotoxic effector functions. Such effects of TGFβ inhibition may further provide long-lasting immunological memory mediated, for example, by CD8+ T cells.

In some embodiments, tumor is poorly immunogenic (e.g., “desert” or “cold” tumors). Patients may benefit from cancer therapy that triggers neo-antigens or promote immune responses. Such therapies include, but are not limited to, chemotherapy, radiation therapy (such as a radiotherapeutic agent), oncolytic viral therapy, oncolytic peptides, tyrosine kinase inhibitors, neo-epitope vaccines, anti-CTLA4, instability inducers, DDR agents, NK cell activators, and various adjuvants such as TLR ligands/agonists. TGFβ1 inhibitors, such as those described herein, can be used in conjunction to boost the effects of cancer therapies. One mode of action for TGFβ1 inhibitors may be to normalize or restore MHC expression, thereby promoting T cell immunity.

Non-limiting examples of TGFβ-related indications include: fibrosis, including organ fibrosis (e.g., kidney fibrosis, liver fibrosis, cardiac/cardiovascular fibrosis, muscle fibrosis, skin fibrosis, uterine fibrosis/endometriosis and lung fibrosis), scleroderma, Alport syndrome, cancer (including, but not limited to: blood cancers such as leukemia, myelofibrosis, multiple myeloma, colon cancer, renal cancer, breast cancer, malignant melanoma, glioblastoma), fibrosis associated with solid tumors (e.g., cancer desmoplasia, such as desmoplastic melanoma, pancreatic cancer-associated desmoplasia and breast carcinoma desmoplasia), stromal fibrosis (e.g., stromal fibrosis of the breast), radiation-induced fibrosis (e.g., radiation fibrosis syndrome), facilitation of rapid hematopoiesis following chemotherapy, bone healing, wound healing, dementia, myelofibrosis, myelodysplasia (e.g., myelodysplasic syndrome or MDS), a renal disease (e.g., end-stage renal disease or ESRD), unilateral ureteral obstruction (UUO), tooth loss and/or degeneration, endothelial proliferation syndromes, asthma and allergy, gastrointestinal disorders, anemia of the aging, aortic aneurysm, orphan indications (such as Marfan's syndrome and Camurati-Engelmann disease), obesity, diabetes, arthritis, multiple sclerosis, muscular dystrophy, bone disorders, amyotrophic lateral sclerosis (ALS), Parkinson's disease, osteoporosis, osteoarthritis, osteopenia, metabolic syndromes, nutritional disorders, organ atrophy, chronic obstructive pulmonary disease (COPD), and anorexia.

Evidence suggests that the ectonucleotidases CD39 and CD73 may at least in part contribute to elevated levels of adenosine in disease conditions. Notably, the CD39/CD73-TGFβ axis may play a role in modulating immune cells implicated in the TGFβ signaling, including Tregs and MDSCs. Both regulatory T cells (Tregs) and myeloid-derived suppressive cells (MDSCs) generally exhibit immunosuppressive phonotypes. In many pathologic conditions (e.g., cancer, fibrosis), these cells are enriched at disease sites and may contribute to creating and/or maintaining an immunosuppressive environment. This may be at least in part mediated by the ectonucleotidases CD39 and CD73 which together participates in the breakdown of ATP into nucleoside adenosine, leading to elevated local concentrations of adenosine in the disease environment, such as tumor microenvironment and fibrotic environment. Adenosine can bind to its receptors expressed on target cells such as T cells and NK cell, which in turn suppress anti-tumor function of these target cells.

Diseases with Aberrant Gene Expression; Biomarkers

It has been observed that abnormal activation of the TGFβ signal transduction pathway in various disease conditions is associated with altered gene expression of a number of markers. These gene expression markers (e.g., as measured by mRNA) include, but are not limited to: Serpine 1 (encoding PAI-1), MCP-1 (also known as CCL2), Col1a1, Col3a1, FN1, TGFB1, CTGF, ACTA2 (encoding α-SMA), SNAI1 (drives EMT in fibrosis and metastasis by downregulating E-cadherin (Cdh1), MMP2 (matrix metalloprotease associated with EMT), MMP9 (matrix metalloprotease associated with EMT), TIMP1 (matrix metalloprotease associated with EMT), FOXP3 (marker of Treg induction), CDH1 (E cadherin (marker of epithelial cells) which is downregulated by TGFβ), and, CDH2 (N cadherin (marker of mesenchymal cells) which is upregulated by TGFβ). Interestingly, many of these genes are implicated to play a role in a diverse set of disease conditions, including various types of organ fibrosis, as well as in many cancers, which include myelofibrosis. Indeed, pathophysiological link between fibrotic conditions and abnormal cell proliferation, tumorigenesis and metastasis has been suggested. See for example, Cox and Erler (2014) Clinical Cancer Research 20(14): 3637-43 “Molecular pathways: connecting fibrosis and solid tumor metastasis”; Shiga et al., (2015) Cancers 7:2443-2458 “Cancer-associated fibroblasts: their characteristics and their roles in tumor growth”; Wynn and Barron (2010) Semin. Liver Dis. 30(3): 245-257 “Macrophages: master regulators of inflammation and fibrosis”, contents of which are incorporated herein by reference. Without wishing to be bound by a particular theory, the inventors of the present disclosure contemplate that the TGFβ1 signaling pathway may in fact be a key link between these broad pathologies.

The ability of chemotactic cytokines (or chemokines) to mediate leukocyte recruitment (e.g., monocytes/macrophages) to injured or disease tissues has crucial consequences in disease progression. Members of the C—C chemokine family, such as monocyte chemoattractant protein 1 (MCP-1), also known as CCL2, macrophage inflammatory protein 1-alpha (MIP-1a), also known as CCL3, and MIP-1p, also known as CCL4, and MIP-2a, also known as CXCL2, have been implicated in this process.

For example, MCP-1/CCL2 is thought to play a role in both fibrosis and cancer. MCP-1/CCL2 is characterized as a profibrotic chemokine and is a monocyte chemoattractant, and evidence suggests that it may be involved in both initiation and progression of cancer. In fibrosis, MCP-1/CCL2 has been shown to play an important role in the inflammatory phase of fibrosis. For example, neutralization of MCP-1 resulted in a dramatic decrease in glomerular crescent formation and deposition of type I collagen. Similarly, passive immunotherapy with either anti-MCP-1 or anti-MIP-1 alpha antibodies is shown to significantly reduce mononuclear phagocyte accumulation in bleomycin-challenged mice, suggesting that MIP-1 alpha and MCP-1 contribute to the recruitment of leukocytes during the pulmonary inflammatory response (Smith, Biol Signals. 1996 July-August; 5(4):223-31, “Chemotactic cytokines mediate leukocyte recruitment in fibrotic lung disease”). Elevated levels of MIP-1alpha in patients with cystic fibrosis and multiple myeloma have been reported (see, for example: Mrugacz et al., J Interferon Cytokine Res. 2007 June; 27(6):491-5), supporting the notion that MIP-1a is associated with localized or systemic inflammatory responses.

Lines of evidence point to the involvement of C—C chemokines in tumor progression/metastasis. For example, tumor-derived MCP-1/CCL2 can promote “pro-cancer” phenotypes in macrophages. For example, in lung cancer, MCP-1/CCL2 has been shown to be produced by stromal cells and promote metastasis. In human pancreatic cancer, tumors secrete CCL2, and immunosuppressive CCR2-positive macrophages infiltrate these tumors. Patients with tumors that exhibit high CCL2 expression/low CD8 T-cell infiltrate have significantly decreased survival. Without wishing to be bound by particular theory, it is contemplated that monocytes that are recruited to an injured or diseased tissue environment may subsequently become polarized in response to local cues (such as in response to tumor-derived cytokines), thereby further contributing to disease progression. These M2-like macrophages are likely to contribute to immune evasion by suppressing effector cells, such as CD4+ and CD8+ T cells. In some embodiments, this process is in part mediated by LRRC33-TGFβ1 expressed by activated macrophages. In some embodiments, the process is in part mediated by GARP-TGFβ1 expressed by Tregs.

Similarly, in certain carcinomas, such as breast cancer (e.g., triple negative breast cancer), CXCL2/CCL22-mediated recruitment of MDSCs has been shown to promote angiogenesis and metastasis (see, for example, Kumar et al., (2018) J Clin Invest 128(11): 5095-5109). It is therefore contemplated that this process is at least in part mediated by TGFβ1, such as LRRC33-TGFβ1. Moreover, because proteases such as MMP9 are implicated in the process of matrix remodeling that contributes to tumor invasion and metastasis, the same or overlapping signaling pathways may also play a role in fibrosis.

Involvement of PAI-1/Serpine1 has been implicated in a variety of fibrotic conditions, cancers, angiogenesis, inflammation, as well as neurodegenerative diseases (e.g., Alzheimer's Disease). Elevated expression of PAI-1 in tumor and/or serum is correlated with poor prognosis (e.g., shorter survival, increased metastasis) in various cancers, such as breast cancer and bladder cancer (e.g., transitional cell carcinoma) as well as myelofibrosis. In the context of fibrotic conditions, PAI-1 has been recognized as an important downstream effector of TGFβ1-induced fibrosis, and increased PAI-1 expression has been observed in various forms of tissue fibrosis, including lung fibrosis (such as IPF), kidney fibrosis, liver fibrosis and scleroderma. In some embodiments, the process is in part mediated by ECM-associated TGFβ1, e.g., via LTBP1-proTGFβ1 and/or LTBP3-proTGFβ1.

In some embodiments, in vivo effects of the TGFβ1 inhibitor therapy may be assessed by measuring changes in expression levels of suitable gene markers. Suitable markers include TGFβ (e.g., TGFB1, TGFβ2, and TGFB3). Suitable markers may also include one or more presenting molecules for TGFβ (e.g., TGFβ1, TGFβ2, and TGFβ3), such as LTBP1, LTBP3, GARP (or LRRC32) and LRRC33. In some embodiments, suitable markers include mesenchymal transition genes (e.g., AXL, ROR2, WNT5A, LOXL2, TWIST2, TAGLN, and/or FAP), immunosuppressive genes (e.g., IL10, VEGFA, VEGFC), monocyte and macrophage chemotactic genes (e.g., CCL2, CCL3, CCL4, CCL7, CCL8, CCL13 and CCL22), and/or various fibrotic markers discussed herein. Exemplary markers are plasma/serum markers.

As previously described in PCT/US2019/041373, isoform-specific, context-independent inhibitors of TGFβ1 can be used to reduce expression levels of many of these markers in suitable preclinical models, including mechanistic animal models, such as UUO, which has been shown to be TGFβ31-dependent. Therefore, such inhibitors may be used to treat a disease or disorder characterized by abnormal expression (e.g., overexpression/upregulation or underexpression/downregulation) of one or more of the gene expression markers of the disease.

Thus, in some embodiments, an isoform-specific, context-independent inhibitor of TGFβ1 is used in the treatment of a disease associated with overexpression of one or more of the following: PAI-1 (encoded by Serpine1), MCP-1 (also known as CCL2), Col1a1, Col3a1, FN1, TGFB1, CTGF, α-SMA, ITGA11, and ACTA2, wherein the treatment comprises administration of the inhibitor to a subject suffering from the disease in an amount effective to treat the disease. In some embodiments, the inhibitor is used to treat a disease associated with overexpression of PAI-1, MCP-1/CCL2, CTGF, and/or α-SMA. In some embodiments, the disease is myelofibrosis. In some embodiments, the disease is cancer, for example, cancer comprising a solid tumor.

Involvement of the TGFβ1 pathway in controlling key facets of both the ECM and immune components may explain the observations that a remarkable number of dysregulated genes are shared across a wide range of pathologies such as proliferative disorders and fibrotic disorders. This supports the notion that the aberrant pattern of expression in the genes involving TGFβ1 signaling is likely a generalizable phenomenon. These marker genes may be classified into several categories such as: genes involved in mesenchymal transition (e.g., EndMT and EMT); genes involved in angiogenesis; genes involved in hypoxia; genes involved in wound healing; and genes involved in tissue injury-triggered inflammatory response.

A comprehensive study carried out by Hugo et al., (Cell, 165(1): 35-44) elegantly demonstrated the correlation between differential gene expression patterns of these classes of markers and the responsiveness to checkpoint blockade therapy (CBT) in metastatic melanoma. The authors found co-enrichment of the set of genes coined “IPRES signatures” defined a transcriptomic subset within not only melanoma, but also all major common human malignancies analyzed. Indeed, the work links tumor cell phenotypic plasticity (i.e., mesenchymal transition) and the resultant impacts on the microenvironment (e.g., ECM remodeling, cell adhesion, and angiogenesis features of immune suppressive wound healing) to CBT resistance. In addition to IPRES, other gene signatures such as TIDE (Jing et al., Nat Med. 2018 October; 24(10):1550-1558), TIS (Danaher et al., J Immunother Cancer. 2018 Jun. 22; 6(1):63), F-TBRS (Mariathasan et al., Nature. 2018 Feb. 22; 554(7693): 544-548), IMPRES (Auslander et al., Nat Med. 2018 October; 24(10): 1545-1549), and xCell (Aran et al. Genome Biol. 2017 Nov. 15; 18(1):220) may also be used to evaluate the tumor immune microenvironment.

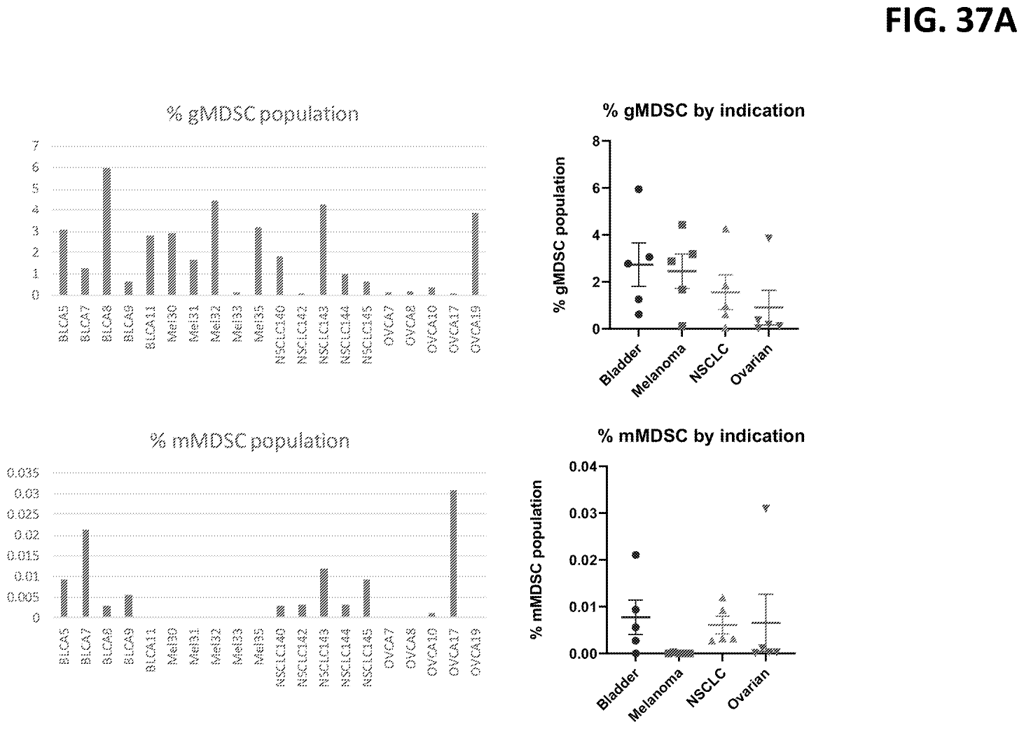

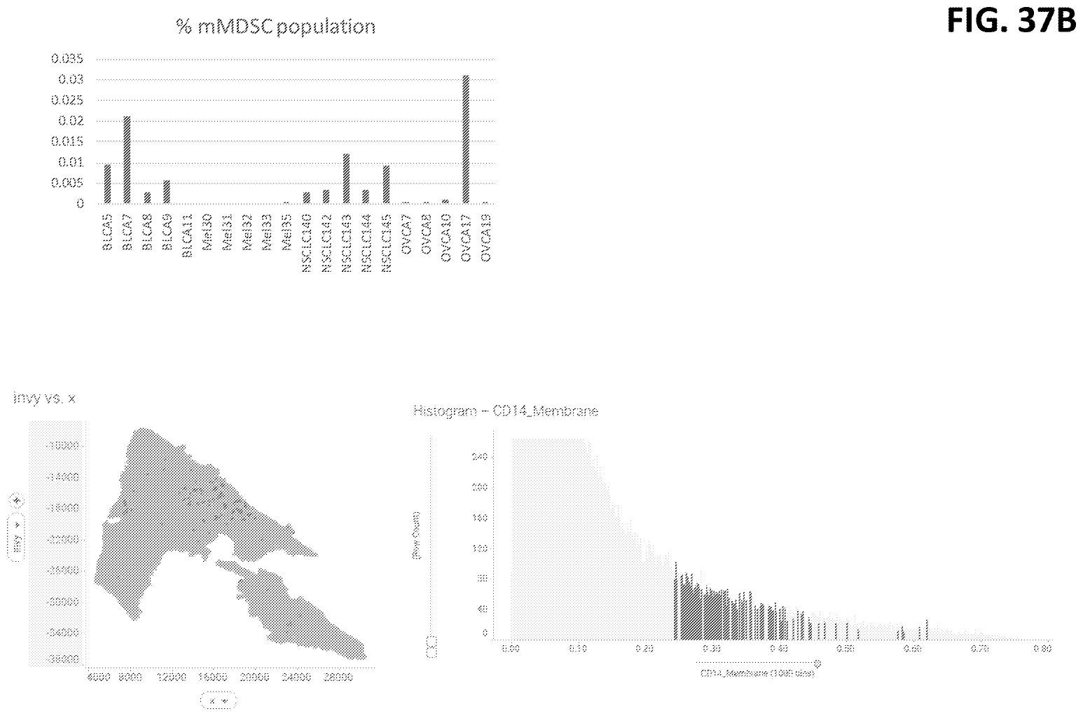

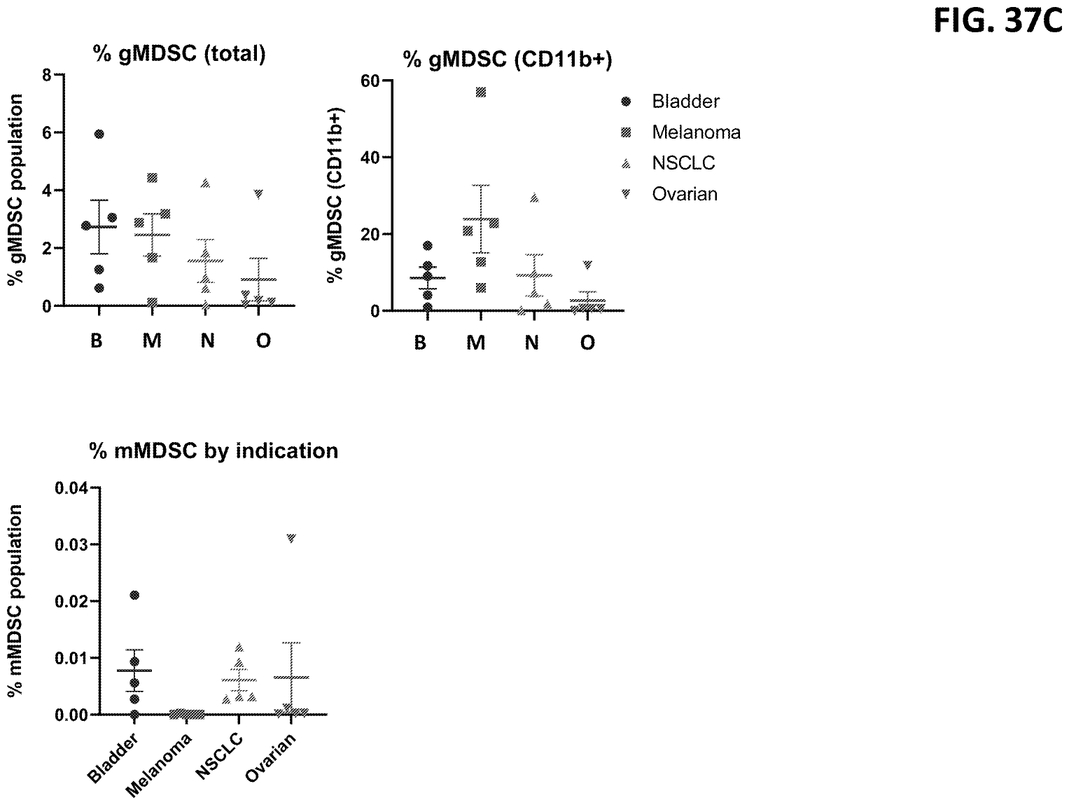

Recognizing that each of these IPRES gene categories has been implicated in disease involving TGFβ dysregulation, Applicant previously contemplated that the TGFβ1 isoform in particular may mediate these processes in disease conditions (see, for example, WO 2017/156500). Work disclosed previously further supported this notion (e.g., PCT/US2019/041373 at Example 11; FIG. 37A), further confirming that therapies that selectively target TGFβ1 (as opposed to non-selective alternatives) may offer an advantage both with respect to efficacy and safety.

Accordingly, the present disclosure includes a method/process of selecting or identifying a candidate patient or patient population likely to respond to a TGFβ1 inhibition therapy, and administering to the patient(s) an effective amount of a high-affinity isoform-selective inhibitor of TGFβ1. Observation of a patient's lack of responsiveness to a CBT (e.g., resistance) may indicate that the patient is a candidate for the TGFβ1 inhibition therapy described herein. Thus, an isoform-selective inhibitor of TGFβ1 such as Ab6 may be used in the treatment of cancer in a subject, wherein the subject is poorly responsive to a CBT. The subject may have advanced cancer, such as a locally advanced solid tumor or metastatic cancer. A patient is said to be “poorly responsive” when there is no or little meaningful therapeutic effects achieved (e.g., do not meet the criteria of partial response or compete response based on standard guidelines, such as RECIST and iRECIST) following a duration of time which is expected to be sufficient to show meaningful therapeutic effects of the particular therapy. Typically, such duration of time for CBTs is at least about 3 months of treatment, either with or without additional therapies such as chemotherapy. Such patients may be referred to as “refractory” or “non-responders.” Where such patients are poorly responsive to the initial CBT, the patients may be referred to as “primary non-responders.” Cancer (or patients with such cancer) in this category may be characterized as having “primary resistance” to the CBT. In some embodiments, the subject is a primary non-responder after receiving at least about 3 months of the CBT treatment, wherein optionally, after at least about 4 months of the CBT treatment. In some embodiments, the subject also received additional therapy in combination with the CBT, such as chemotherapy.

Upon identification of the subject as a non-responder of a CBT, the high-affinity, isoform-selective inhibitor of TGFβ1 may be administered to the subject in conjunction with a CBT, which may or may not comprise the same checkpoint inhibitor as the first CBT to which the subject failed to respond. Any suitable immune checkpoint inhibitors may be used, e.g., approved checkpoint inhibitors. In some embodiments, the high-affinity, isoform-selective inhibitor of TGFβ1 is administered to the subject in conjunction with a CBT comprising an anti-PD-1 antibody or anti-PD-L1 antibody. The high-affinity, isoform-selective inhibitor of TGFβ1 is aimed to overcome the resistance by rendering the cancer more susceptible to the CBT.

The process of selecting or identifying a candidate patient or patient population likely to respond to, or otherwise likely to benefit from, a TGFβ1 inhibition therapy may comprise a step of testing a biological sample collected from the patient (or patient population), such as biopsy samples, for the expression of one or more of the markers discussed herein. Similarly, such genetic marker(s) may be used for purposes of monitoring the patient's responsiveness to a therapy. Monitoring may include testing two or more biological samples collected from the patient, for example, before and after administration of a therapy, and during the course of a therapeutic regimen over time, to evaluate changes in gene expression levels of one or more of the markers, indicative of therapeutic response or effectiveness. In some embodiments, a liquid biopsy may be used.

In some embodiments, a method of selecting a candidate patient or patient population likely to respond to a TGFβ1 inhibition therapy may comprise a step of identifying a patient or patient population previously tested for the genetic marker(s), such as those described herein, which showed aberrant expression thereof. These same methods are also applicable to later confirming or correlating with the patients' response to the therapy.

In some embodiments, the aberrant marker expression includes elevated levels of at least one of the following: TGFβ1, LRRC33, GARP, LTBP1, LTBP3, CCL2, CCL3, PAI-1/Serpine1. In some embodiments, the patient or patient population (e.g., biological samples collected therefrom) shows elevated TGFβ1 activation, phospho-Smad2, phospho-Smad2/3, or combination thereof. In some embodiments, the patient or patient population (e.g., biological samples collected therefrom) shows elevated MDSCs. In some embodiments, such patient or patient population has cancer, which may comprise a solid tumor that is TGFβ1-positive. The solid tumor may be a TGFβ1-dominant tumor, in which TGFβ1 is the predominant isoform expressed in the tumor, relative to the other isoforms. In some embodiments, the solid tumor may be a TGFβ1-co-dominant tumor, in which TGFβ1 is the co-dominant isoform expressed in the tumor, e.g., TGFβ1+/TGFβ3+. In some embodiments, such patient or patient population exhibits resistance to a cancer therapy, such as chemotherapy, radiation therapy (such as a radiotherapeutic agent) and/or immune checkpoint therapy, e.g., anti-PD-1 (e.g., pembrolizumab, nivolumab, spartalizumab), anti-PD-L1 (e.g., atezolizumab), anti-CTLA4 (e.g., ipilimumab), engineered immune cell therapy (e.g., CAR-T), and cancer vaccines, etc. According to the disclosure, TGFβ1 inhibitors provided herein, such as Ab6, overcome the resistance by unblocking immunosuppression so as to allow effector cells to gain access to cancer cells thereby achieving anti-tumor effects. TGFβ1 inhibitor therapy may therefore promote effector cell infiltration and/or expansion in the tumor. Additionally, TGFβ1 inhibitor therapy may reduce the frequency of immunosuppressive immune cells, such as Tregs and MDSCs, in the tumor.

In some embodiments, the aberrant marker expression includes one or more panels of genes: mesenchymal transition markers (e.g., AXL, ROR2, WNT5A, LOXL2, TWIST2, TAGLN, FAP); immunosuppressive genes (e.g., IL10, VEGFA, VEGFC); monocyte and macrophage chemotactic genes (e.g., CCL2, CCL7, CCL8, CCL13); genes involved in angiogenesis and wound healing (e.g., T cell suppressive); cell adhesion markers; ECM remodeling; skeletal system and bone development markers; and genes involved in tissue injury-triggered inflammatory response.

In some embodiments, lack or downregulation of MHC expression (such as MHC class 1) may serve as a biomarker for TGFβ1-associated conditions for which the antibodies or antigen-binding fragments encompassed by the present disclosure may be used as therapy. Reduced MHC levels may signal immune escape, which may correlate with poor responsiveness of the patients to immune therapies, such as CBT. Selective inhibition of TGFβ1 therefore may at least in part restore effector cell function.

The present disclosure provides a TGFβ inhibitor (e.g., TGFβ1-selective inhibitor such as Ab6) for use in the treatment of a TGFβ-related disorder with aberrant gene expression (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Diseases Involving Mesenchymal Transition

Mesenchymal transition is a process of phenotypic shift of cells, such as epithelial cells and endothelial cells, towards a mesenchymal phenotype (such as myofibroblasts). Examples of genetic markers indicative of mesenchymal transition include AXL, ROR2, WNT5, LOXL2, TWIST2, TAGLN and FAP. In cancer, for example, mesenchymal transition (e.g., increased EndMT and EMT signatures) indicates tumor cell phenotypic plasticity. Thus, inhibitors of TGFβ, e.g., TGFβ1 inhibitors, such as Ab6, may be used to treat a disease that is initiated or driven by mesenchymal transition, such as EMT and EndMT.

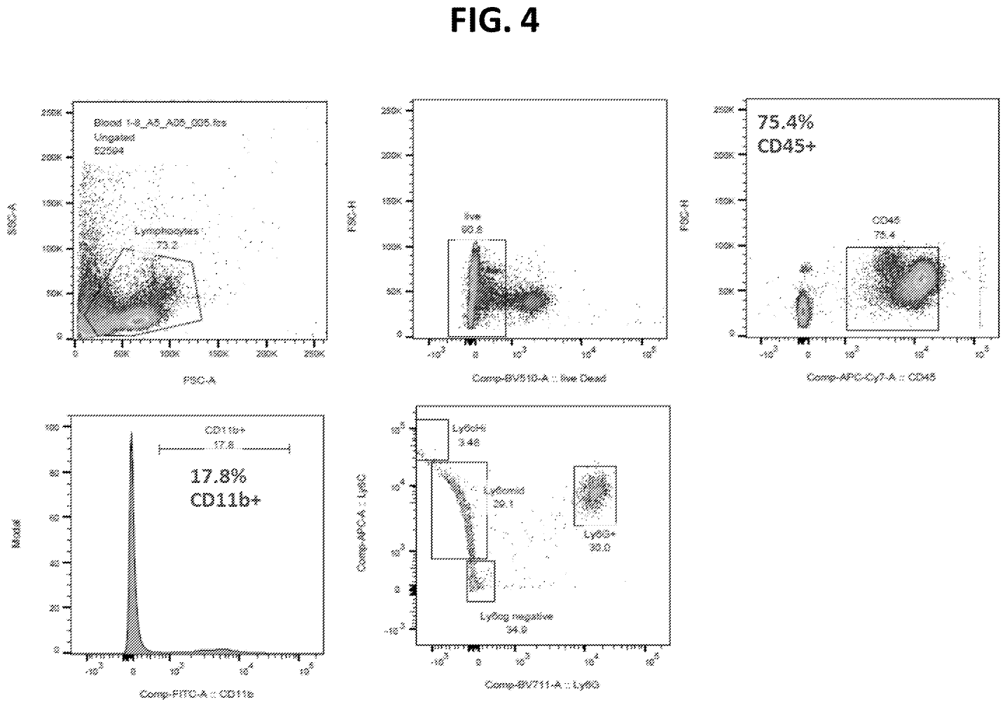

EMT (epithelial-to-mesenchymal transition) is the process by which epithelial cells with tight junctions switch to mesenchymal properties (phenotypes) such as loose cell-cell contacts. The process is observed in a number of normal biological processes as well as pathological situations, including embryogenesis, wound healing, cancer metastasis and fibrosis (reviewed in, for example, Shiga et al., (2015) “Cancer-Associated Fibroblasts: Their Characteristics and Their Roles in Tumor Growth.” Cancers, 7: 2443-2458). Generally, it is believed that EMT signals are induced mainly by TGFβ. Many types of cancer, for example, appear to involve transdifferentiation of cells towards mesenchymal phenotype (such as myofibroblasts and CAFs) which correlate with poorer prognosis. Thus, isoform-specific, context-independent inhibitors of TGFβ1, such as those described herein, may be used to treat a disease that is initiated or driven by EMT. Indeed, data exemplified in PCT/US2019/041373 (e.g., FIGS. 4-6) show that such inhibitors have the ability to suppress expression of myofibroblast/CAF markers in vivo, such as α-SMA, LOXL2, Col1 (Type I collagen), and FN (fibronectin). Thus, TGFβ inhibitors, e.g., TGFβ1 inhibitors, such as Ab6, may be used for the treatment of a disease characterized by EMT. A therapeutically effective amount of the inhibitor may be an amount sufficient to reduce expression of markers such as α-SMA/ACTA2, LOXL2Col1 (Type I collagen), and FN (fibronectin). In some embodiments, the disease is a proliferative disorder, such as cancer.

Similarly, TGFβ is also a key regulator of the endothelial-to-mesenchymal transition (EndMT) observed in normal development, such as heart formation. However, the same or similar phenomenon is also seen in many disease-associated tissues, such as cancer stroma and fibrotic sites. In some disease processes, endothelial markers such as CD31 become downregulated upon TGFβ1 exposure and instead the expression of mesenchymal markers such as FSP-1, α-SMA/ACTA2 and fibronectin becomes induced. Indeed, stromal CAFs may be derived from vascular endothelial cells. Thus, TGFβ inhibitors, e.g., TGFβ1 inhibitors, such as Ab6, may be used for the treatment of a disease characterized by EndMT. A therapeutically effective amount of the inhibitor may be an amount sufficient to reduce expression of markers such as FSP-1, α-SMA/ACTA2 and fibronectin. In some embodiments, the disease is a proliferative disorder, such as cancer.

The present disclosure provides a TGFβ inhibitor (e.g., TGFβ31-selective inhibitor such as Ab6) for use in the treatment of a TGFβ-related disorder involving mesenchymal transition (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Diseases Involving Matrix Stiffening and Remodeling

Progression of various TGFβ31-related indications, such as fibrotic conditions and cancer (e.g., tumor growth and metastasis), involves increased levels of matrix components deposited into the ECM and/or maintenance/remodeling of the ECM. It has been reported that increased deposition of ECM components such as collagens can alter the mechanophysical properties of the ECM (e.g., the stiffness of the matrix/substrate) and this phenomenon is associated with TGFβ1 signaling. Applicant previously demonstrated the role of matrix stiffness on integrin-dependent activation of TGFβ, using primary fibroblasts transfected with proTGFβ1 and LTBP1 and grown on silicon-based substrates with defined stiffness (e.g., 5 kPa, 15 kPa or 100 kPa). As disclosed in WO 2018/129329, matrices with greater stiffness enhance TGFβ1 activation, and this can be suppressed by isoform-specific inhibitors of TGFβ1. These observations suggest that TGFβ1 influences ECM properties (such as stiffness), which in turn can further induce TGFβ1 activation, reflective of disease progression.

Thus, TGFβ1 inhibitors, such as Ab6, may be used to block this process to counter disease progression involving ECM alterations, such as fibrosis, tumor growth, invasion, metastasis and desmoplasia. The LTBP-arm of such inhibitors can directly target ECM-associated pro/latent TGFβ1 complexes which are presented by LTBP1 and/or LTBP3, thereby preventing activation/release of the growth factor from the complex in the disease niche. In some embodiments, the TGFβ1 inhibitors may normalize ECM stiffness to treat a disease that involves integrin-dependent signaling. In some embodiments, the integrin comprises an all chain, 31 chain, or both. The architecture of the ECM, e.g., ECM components and organization, can also be altered by matrix-associated proteases. Thus, in some embodiments, the TGFβ1 inhibitors may normalize ECM stiffness to treat a disease that involves protease-dependent signaling associated with disease-associated ECM, e.g., in tumor and fibrotic tissues.

As reviewed in Lampi and Reinhart-King (Science Translational Medicine, 10(422): eaao0475, “Targeting extracellular matrix stiffness to attenuate disease: From molecular mechanisms to clinical trials”), increased stiffness of tissue ECMs occurs during pathological progression of cancer, fibrosis and cardiovascular disease. The mechanical properties associated with the process involve phenotypically converted myofibroblasts, TGFβ and matrix cross-linking. A major cause of increased ECM stiffness during cancer and fibrotic diseases is dysregulated matrix synthesis and remodeling by activated fibroblasts that have de-differentiated into myofibroblasts (e.g., CAFs and FAFs). Remodeling of the tumor stroma and organ fibrosis exhibit striking similarities to the wound healing response, except that in the pathological state the response is sustained. Myofibroblasts are a heterogeneous cell population with pathology-specific precursor cells originating from multiple cell sources, such as bone marrow-derived and tissue resident cells. Commonly used myofibroblast markers include alpha-smooth muscle actin (α-SMA). As previously shown in PCT/US2019/041373, high-affinity, isoform-specific TGFβ1 inhibitors are able to reduce ACTA2 expression (which encodes α-SMA), collagens, as well as FN (fibronectin) in in vivo studies. Fibronectin is important in the anchoring of LTBP-associated proTGFβ1 complexes onto the matrix structure.

The importance of the TGFβ pathway in ECM regulation is well-established. Because TGFβ1 (and TGFβ3) can be mechanically activated by certain integrins (e.g., αv integrins), the integrin-TGFβ1 interaction has become a therapeutic target. For example, a monoclonal antibody to αvβ6 has been investigated for idiopathic lung fibrosis. However, such approach is expected to also interfere with TGFβ3 signaling which shares the same integrin-binding motif, RGD, and furthermore, such antibody will not be effective in blocking TGFβ1 activated via other modes, such as protease-induced activation. In comparison, high-affinity, isoform-specific TGFβ1 inhibitors, such as Ab6, can also block protease-dependent activation of TGFβ1, as well as integrin-dependent activation of TGFβ1. Therefore, such TGFβ1 inhibitors may provide superior attributes. Based on Applicant's previous work, high-affinity isoform-selective inhibitors of TGFβ1 may be effective in treating disease associated with ECM stiffening.

Thus, the disclosure includes therapeutic use of isoform-selective inhibitors of TGFβ1 in the treatment of a disease associated with matrix stiffening, or in a method for reducing matrix stiffness, in a subject. Such use comprises administration of a therapeutically effective amount of the isoform-selective inhibitor of TGFβ1, such as Ab6.

The present disclosure provides a TGFβ inhibitor (e.g., TGFβ1-selective inhibitor such as Ab6) for use in the treatment of a TGFβ-related disorder involving matrix stiffening and remodeling (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Diseases Involving Proteases

Activation of TGFβ from its latent complex may be triggered mechanically by integrin in a force-dependent manner, and/or by proteases. Evidence suggests that certain classes of proteases may be involved in the process, including but are not limited to Ser/Thr proteases such as Kallikreins, chemotrypsin, elastases, plasmin, thrombin, as well as zinc metalloproteases of MMP family, such as MMP-2, MMP-9 and MMP-13, and the Adam family of proteases, such as Adam10 and Adam17. MMP-2 degrades the most abundant component of the basement membrane, Collagen IV, raising the possibility that it may play a role in ECM-associated TGFβ1 regulation. MMP-9 has been implicated to play a central role in tumor progression, angiogenesis, stromal remodeling and metastasis, including in carcinoma, such as breast cancer. Thus, protease-dependent activation of TGFβ1 in the ECM may be important for treating ECM-associated diseases such as fibrosis and cancer.

Kallikreins (KLKs) are trypsin- or chymotrypsin-like serine proteases that include plasma Kallikreins and tissue Kallikreins. The ECM plays a role in tissue homeostasis acting as a structural and signaling scaffold and barrier to suppress malignant outgrowth. KLKs may play a role in degrading ECM proteins and other components which may facilitate tumor expansion and invasion. For example, KLK1 is highly upregulated in certain breast cancers and can activate pro-MMP-2 and pro-MMP-9. KLK2 activates latent TGFβ1, rendering prostate cancer adjacent to fibroblasts permissive to cancer growth. KLK3 has been widely studied as a diagnostic marker for prostate cancer (PSA). KLK3 may directly activate TGFβ1 by processing plasminogen into plasmin, which proteolytically cleaves LAP, thereby causing the TGFβ1 growth factor to be released from the latent complex. KLK6 may be a potential marker for Alzheimer's disease.

Moreover, data previously provided by Applicant indicated that such proteases may be a Kallikrein. Thus, the disclosure encompasses the use of an isoform-specific, context-independent inhibitor of TGFβ1 in a method for treating a disease associated with Kallikrein or a Kallikrein-like protease. In some embodiments, the TGFβ1 inhibitor is Ab6, or derivatives thereof.

Known activators of TGFβ1, such as plasmin, TSP-1 and αVβ6 integrin, all interact directly with LAP. It is postulated that proteolytic cleavage of LAP may destabilize the LAP-TGFβ interaction, thereby releasing active TGFβ1 (the growth factor domain) from the latent complex. It has been suggested that the region containing the amino acid stretch 54-LSKLRL-59 (SEQ ID NO: 171) is important for maintaining TGFβ1 latency. Thus, agents (e.g., antibodies) that stabilize the interaction, or block the proteolytic cleavage of LAP may prevent TGFβ1 activation.

Many of these proteases associated with pathological conditions (e.g., cancer) function through distinct mechanisms of action. Thus, targeted inhibition of particular proteases, or combinations of proteases, may provide therapeutic benefits for the treatment of conditions involving the protease-TGFβ axis. Accordingly, it is contemplated that inhibitors (e.g., TGFβ1 antibodies) that selectively inhibit protease-induced activation of TGFβ1 may be advantageous in the treatment of such diseases (e.g., cancer). Similarly, selective inhibition of TGFβ1 activation by one protease over another protease may also provide therapeutic benefit, depending on the condition being treated.

Plasmin is a serine protease produced as a precursor form called Plasminogen. Upon release, Plasmin enters circulation and therefore is detected in serum. Elevated levels of serum Plasmin appear to correlate with cancer progression, possibly through mechanisms involving disruption of the extracellular matrix (e.g., basement membrane and stromal barriers) which facilitates tumor cell motility, invasion, and metastasis. Plasmin may also affect adhesion, proliferation, apoptosis, cancer nutrition, oxygen supply, formation of blood vessels, and activation of VEGF (Didiasova et al., Int. J. Mol. Sci, 2014, 15, 21229-21252). In addition, Plasmin may promote the migration of macrophages into the tumor microenvironment (Philips et al., Cancer Res. 2011 Nov. 1; 71(21):6676-83 and Choong et al., Clin. Orthop. Relat. Res. 2003, 415S, S46-S58). Indeed, tumor-associated macrophages (TAMs) are well characterized drivers of tumorigenesis through their ability to promote tumor growth, invasion, metastasis, and angiogenesis.

Plasmin activities have been primarily tied to the disruption of the ECM. However, there is mounting evidence that Plasmin also regulates downstream MMP and TGFβ activation. Specifically, Plasmin has been suggested to cause activation of TGFβ through proteolytic cleavage of the Latency Associated Peptide (LAP), which is derived from the N-terminal region of the TGFβ gene product (Horiguchi et al., J Biochem. 2012 October; 152(4):321-9), resulting in the release of active growth factor. Since TGFβ1 may promote cancer progression, this raises the possibility that plasmin-induced activation of TGFβ may at least in part mediate this process.

TGFβ1 has also been shown to regulate expression of uPA, which is a critical player in the conversion of Plasminogen into Plasmin (Santibanez, Juan F., ISRN Dermatology, 2013: 597927). uPA has independently been shown to promote cancer progression (e.g., adhesion, proliferation, and migration) by binding to its cell surface receptor (uPAR) and promoting conversion of Plasminogen into Plasmin. Moreover, studies have shown that expression of uPA and/or plasminogen activator inhibitor-1 (PAI-1) are predictors of poor prognosis in colorectal cancer (D. Q. Seetoo, et al., Journal of Surgical Oncology, vol. 82, no. 3, pp. 184-193, 2003), breast cancer (N. Harbeck et al., Clinical Breast Cancer, vol. 5, no. 5, pp. 348-352, 2004), and skin cancer (Santibanez, Juan F., ISRN Dermatology, 2013: 597927). Thus, without wishing to be bound by a particular theory, the interplay between Plasmin, TGFβ1, and uPA may create a positive feedback loop towards promoting cancer progression. Accordingly, inhibitors that selectively inhibit Plasmin-dependent TGFβ1 activation may be particularly suitable for the treatment of cancers reliant on the Plasmin/TGFβ1 signaling axis.

In one aspect of the disclosure, TGFβ inhibitors such as the isoform-specific inhibitors of TGFβ1 described herein can inhibit protease-dependent activation of TGFβ1. In some embodiments, the inhibitors can inhibit protease-dependent TGFβ1 activation in an integrin-independent manner. In some embodiments, such inhibitors can inhibit TGFβ1 activation irrespective of the mode of activation, e.g., inhibit both integrin-dependent activation and protease-dependent activation of TGFβ1. In some embodiments, the protease is selected from the group consisting of: serine proteases, such as Kallikreins, Chemotrypsin, Trypsin, Elastases, Plasmin, as well as zinc metalloproteases (MMP family) such as MMP-2, MMP-9 and MMP-13.

In some embodiments, the TGFβ inhibitors (e.g., TGFβ1 antibody) can inhibit Plasmin-induced activation of TGFβ1. In some embodiments, the inhibitors can inhibit Plasmin- and integrin-induced TGFβ1 activation. In some embodiments, the antibody is a monoclonal antibody that specifically binds proTGFβ1. In some embodiments, the antibody binds latent proTGFβ1 thereby inhibiting release of mature growth factor from the latent complex. In some embodiments, the high-affinity, context-independent inhibitor of TGFβ1 activation suitable for use in the method of inhibiting Plasmin-dependent activation of TGFβ1 is Ab6 or a derivative or variant thereof.

In some embodiments, the TGFβ inhibitor (e.g., TGFβ1 antibody) inhibits cancer cell migration. In some embodiments, the inhibitor inhibits macrophage migration. In some embodiments, the inhibitor inhibits accumulation of TAMs.

In another aspect, provided herein is a method for treating cancer in a subject in need thereof, the method comprising administering to the subject an effective amount of an TGFβ inhibitor (e.g., TGFβ1 antibody), wherein the inhibitor inhibits protease-induced activation of TGFβ1 (e.g., Plasmin), thereby treating cancer in the subject.

In another aspect, provided herein is a method of reducing tumor growth in a subject in need thereof, the method comprising administering to the subject an effective amount of an TGFβ inhibitor (e.g., TGFβ1 antibody), wherein the inhibitor inhibits protease-induced activation of TGFβ1 (e.g., Plasmin), thereby reducing tumor growth in the subject.

The present disclosure provides a TGFβ inhibitor (e.g., TGFβ1-selective inhibitor such as Ab6) for use in the treatment of a TGFβ-related disorder involving protease(s) (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Myeloproliferative Disorders/Myelofibrosis

The present disclosure provides therapeutic use of TGFβ1 inhibitors, such as Ab6, in the treatment of myeloproliferative disorders. These include, for example, myelodysplastic syndrome (MDS) and myelofibrosis (e.g., primary myelofibrosis and secondary myelofibrosis).

Myelofibrosis, also known as osteomyelofibrosis, is a relatively rare bone marrow proliferative disorder (cancer), which belongs to a group of diseases called myeloproliferative disorders. Myelofibrosis is classified into the Philadelphia chromosome-negative (−) branch of myeloproliferative neoplasms. Myelofibrosis is characterized by clonal myeloproliferation, aberrant cytokine production, extramedullary hematopoiesis, and bone marrow fibrosis. The proliferation of an abnormal clone of hematopoietic stem cells in the bone marrow and other sites results in fibrosis, or the replacement of the marrow with scar tissue. The term myelofibrosis, unless otherwise specified, refers to primary myelofibrosis (PMF). This may also be referred to as chronic idiopathic myelofibrosis (cIMF) (the terms idiopathic and primary mean that in these cases the disease is of unknown or spontaneous origin). This is in contrast with myelofibrosis that develops secondary to polycythemia vera or essential thrombocythaemia. Myelofibrosis is a form of myeloid metaplasia, which refers to a change in cell type in the blood-forming tissue of the bone marrow, and often the two terms are used synonymously. The terms agnogenic myeloid metaplasia and myelofibrosis with myeloid metaplasia (MMM) are also used to refer to primary myelofibrosis. In some embodiments, the hematologic proliferative disorders which may be treated in accordance with the present disclosure include myeloproliferative disorders, such as myelofibrosis. So-called “classical” group of BCR-ABL (Ph) negative chronic myeloproliferative disorders includes essential thrombocythemia (ET), polycythemia vera (PV) and primary myelofibrosis (PMF).

Myelofibrosis disrupts the body's normal production of blood cells. The result is extensive scarring in the bone marrow, leading to severe anemia, weakness, fatigue and often an enlarged spleen. Production of cytokines such as fibroblast growth factor by the abnormal hematopoietic cell clone (particularly by megakaryocytes) leads to replacement of the hematopoietic tissue of the bone marrow by connective tissue via collagen fibrosis. The decrease in hematopoietic tissue impairs the patient's ability to generate new blood cells, resulting in progressive pancytopenia, a shortage of all blood cell types. However, the proliferation of fibroblasts and deposition of collagen is thought to be a secondary phenomenon, and the fibroblasts themselves may not be part of the abnormal cell clone.

Myelofibrosis may be caused by abnormal blood stem cells in the bone marrow. The abnormal stem cells produce mature and poorly differentiated cells that grow quickly and take over the bone marrow, causing both fibrosis (scar tissue formation) and chronic inflammation.

Primary myelofibrosis is associated with mutations in Janus kinase 2 (JAK2), thrombopoietin receptor (MPL) and calreticulin (CALR), which can lead to constitutive activation of the JAK-STAT pathway, progressive scarring, or fibrosis, of the bone marrow occurs. Patients may develop extramedullary hematopoiesis, i.e., blood cell formation occurring in sites other than the bone marrow, as the haemopoetic cells are forced to migrate to other areas, particularly the liver and spleen. This causes an enlargement of these organs. In the liver, the abnormal size is called hepatomegaly. Enlargement of the spleen is called splenomegaly, which also contributes to causing pancytopenia, particularly thrombocytopenia and anemia. Another complication of extramedullary hematopoiesis is poikilocytosis, or the presence of abnormally shaped red blood cells.

The principal site of extramedullary hematopoiesis in myelofibrosis is the spleen, which is usually markedly enlarged in patients suffering from myelofibrosis. As a result of massive enlargement of the spleen, multiple subcapsular infarcts often occur in the spleen, meaning that due to interrupted oxygen supply to the spleen partial or complete tissue death happens. On the cellular level, the spleen contains red blood cell precursors, granulocyte precursors and megakaryocytes, with the megakaryocytes prominent in their number and in their abnormal shapes. Megakaryocytes may be involved in causing the secondary fibrosis seen in this condition.

It has been suggested that TGFβ may be involved in the fibrotic aspect of the pathogenesis of myelofibrosis (see, for example, Agarwal et al., “Bone marrow fibrosis in primary myelofibrosis: pathogenic mechanisms and the role of TGFβ” (2016) Stem Cell Investig 3:5). Bone marrow pathology in primary myelofibrosis is characterized by fibrosis, neoangeogenesis and osteosclerosis, and the fibrosis is associated with an increase in production of collagens deposited in the ECM.

A number of biomarkers have been described, alternations of which are indicative of or correlate with the disease. In some embodiments, the biomarkers are cellular markers. Such disease-associated biomarkers are useful for the diagnosis and/or monitoring of the disease progression as well as effectiveness of therapy (e.g., patients' responsiveness to the therapy). These biomarkers include a number of fibrotic markers, as well as cellular markers. In lung cancer, for example, TGFβ1 concentrations in the bronchoalveolar lavages (BAL) fluid are reported to be significantly higher in patients with lung cancer compared with patients with benign diseases (˜2+ fold increase), which may also serve as a biomarker for diagnosing and/or monitoring the progression or treatment effects of lung cancer.

Because myelofibrosis is associated with abnormal megakaryocyte development, certain cellular markers of megakaryocytes as well as their progenitors of the stem cell lineage may serve as markers to diagnose and/or monitor the disease progression as well as effectiveness of therapy. In some embodiments, useful markers include, but are not limited to: cellular markers of differentiated megakaryocytes (e.g., CD41, CD42 and Tpo R), cellular markers of megakaryocyte-erythroid progenitor cells (e.g., CD34, CD38, and CD45RA−), cellular markers of common myeloid progenitor cells (e.g., IL-3a/CD127, CD34, SCF R/c-kit and Flt-3/FIk-2), and cellular markers of hematopoietic stem cells (e.g., CD34, CD38−, Flt-3/FIk-2). In some embodiments, useful biomarkers include fibrotic markers. These include, without limitation: TGFβ1/TGFB1, PAI-1 (also known as Serpine1), MCP-1 (also known as CCL2), Collal, Col3a1, FN1, CTGF, α-SMA, ACTA2, Timp1, Mmp8, and Mmp9. In some embodiments, useful biomarkers are serum markers (e.g., proteins or fragments found and detected in serum samples).

Based on the finding that TGFβ is a component of the leukemic bone marrow niche, it is contemplated that targeting the bone marrow microenvironment with TGFβ inhibitors may be a promising approach to reduce leukemic cells expressing presenting molecules that regulate local TGFβ availability in the effected tissue.

Indeed, due to the multifaceted nature of the pathology which manifests TGFβ-dependent dysregulation in both myelo-proliferative and fibrotic aspects (as the term “myelofibrosis” itself suggests), isoform-specific, TGFβ inhibitors such as those described herein may provide particularly advantageous therapeutic effects for patients suffering from myelofibrosis. It is contemplated that the LTBP-arm of such inhibitor can target ECM-associated TGFβ1 complex in the bone marrow, whilst the LRRC33-arm of the inhibitor can block myeloid cell-associated TGFβ1. In addition, abnormal megakaryocyte biology associated with myelofibrosis may involve both GARP- and LTBP-mediated TGFβ1 activities. Thus, TGFβ inhibitors such as the isoform-specific, context-independent inhibitor of TGFβ1 disclosed herein, may be capable of targeting such complexes and thereby inhibiting release of active TGFβ1 in the niche.

TGFβ inhibitors such as the TGFβ1-selective inhibitors described herein are useful for treatment of patients with primary and secondary myelofibrosis, who have had an inadequate response to or are intolerant of other (or standard-of-care) treatments, such as hydroxyurea and JAK inhibitors. Such inhibitors are also useful for treatment of patients with intermediate or high-risk myelofibrosis (MF), including primary MF, post-polycythemia vera MF and post-essential thrombocythemia MF. In some embodiments, such TGFβ inhibitors may be used in combination with a checkpoint inhibitor therapy.

Accordingly, one aspect of the disclosure relates to methods for treating primary myelofibrosis. The method comprises administering to a patient suffering from primary myelofibrosis a therapeutically effective amount of a composition comprising a TGFβ inhibitorthat causes reduced TGFβ availability. In some embodiments, an isoform-specific, context-context-independent monoclonal antibody inhibitor of TGFβ1 activation is administered to patients with myelofibrosis. Such antibody may be administered at dosages ranging between 0.1 and 100 mg/kg, such as between 1 and 30 mg, e.g., 1 mg/kg, 3 mg/kg, 5 mg/kg, 10 mg/kg, 15 mg/kg, 20 mg/kg, 30 mg/kg, etc. For example, suitable dosing regimens include between 1-30 mg/kg administered weekly. In some embodiments, the TGFβ1 inhibitor is dosed at about 10 mg/kg per week. Optionally, the frequency of administration may be adjusted after the initial phase, for example, from about once a week (during an initial phase) to once a month (during a maintenance phase). In some embodiments, the TGFβ inhibitor (e.g., a TGFβ1 inhibitor) may be administered in combination with a checkpoint inhibitor therapy.

Exemplary routes of administration of a pharmaceutical composition comprising the antibody is intravenous or subcutaneous administration. When the composition is administered intravenously, the patient may be given the therapeutic over a suitable duration of time, e.g., approximately 30-120 minutes (e.g., 30 min, 60 min, 75 min, 90 min, and 120 min), per treatment, and then repeated every several weeks, e.g., 3 weeks, 4 weeks, 6 weeks, etc., for a total of several cycles, e.g., 4 cycles, 6, cycles, 8 cycles, 10 cycles, 12 cycles, etc. In some embodiments, patients are treated with a composition comprising the inhibitory antibody at dose level of 1-10 mg/kg (e.g., 1, 2, 3, 4, 5, 6, 7, 8, 9, or 10 mg/kg per dosing) via intravenous administration every 28 days (4 weeks) for 6 cycles or 12 cycles. In some embodiments, such treatment is administered as a chronic (long-term) therapy (e.g., to be continued indefinitely, as long as deemed beneficial) in lieu of discontinuing following a set number of cycles of administration.

While myelofibrosis is considered a type of leukemia, it is also characterized by the manifestation of fibrosis. Because TGFβ is known to regulate aspects of ECM homeostasis, the dysregulation of which can lead to tissue fibrosis, it is desirable to inhibit TGFβ activities associated with the ECM. Accordingly, antibodies or fragments thereof that bind and inhibit proTGFβ presented by LTBPs (such as LTBP1 and LTBP3) are encompassed by this disclosure. In some embodiments, antibodies or fragments thereof suitable for treating myelofibrosis are “context-independent” in that they can bind multiple contexts of proTGFβ complex, such as those associated with LRRC33, GARP, LTBP1, LTBP3, or any combination thereof. In some embodiments, such antibody is a context-independent inhibitor of TGFβ activation, characterized in that the antibody can bind and inhibit any of the following latent complexes: LTBP1-proTGFβ, LTBP3-proTGFβ, GARP-proTGFβ and LRRC33-proTGFβ. In some embodiments, such an antibody is an isoform-specific antibody that binds and inhibits such latent complexes that comprise one but not the other isoforms of TGFβ. These include, for example, LTBP1-proTGFβ1, LTBP3-proTGFβ1, GARP-proTGFβ1 and LRRC33-proTGFβ1. In some embodiments, such antibody is an isoform-selective antibody that preferentially binds with high affinity and inhibits TGFβ1 signaling.

Early in vivo data indicate that TGFβ inhibitors such as an isoform-selective context-independent inhibitor of TGFβ1 described herein, can be used to treat myelofibrosis in a translatable murine model of primary myelofibrosis. Unlike the current standard of care JAK2 inhibitor, which only provides symptomic relief but does not provide clinical or survival benefits, the TGFβ inhibitor (e.g., an isoform-selective context-independent inhibitor of TGFβ1 described herein) achieves significant anti-fibrotic effects in the bone marrow of the diseased mice and may also prolong survival, supporting the notion that the TGFβ1 inhibitor may be effective to treat myeloproliferative disorders in human patients.

Suitable patient populations of myeloproliferative neoplasms who may be treated with the compositions and methods described herein may include, but are not limited to: a) a patient population that is Philadelphia (+); b) a patient population that is Philadelphia (−); c) a patient population that is categorized “classical” (PV, ET and PMF); d) a patient population carrying the mutation JAK2V617F(+); e) a patient population carrying JAK2V617F(−); f) a patient population with JAK2 exon 12(+); g) a patient population with MPL(+); and h) a patient population with CALR(+).

In some embodiments, the patient population includes patients with intermediate-2 or high-risk myelofibrosis. In some embodiments, the patient population comprises subjects with myelofibrosis who are refractory to or not candidates for available therapy. In some embodiments, the subject has platelet counts between 100-200×10⁹/L. In some embodiments, the subject has platelet counts >200×10⁹/L prior to receiving the treatment.

In some embodiments, a subject to receive (and who may benefit from receiving) an isoform-specific, context-independent TGFβ1 inhibitor therapy is diagnosed with intermediate-1 or higher primary myelofibrosis (PMF), or post-polycythemmia vera/essential thrombocythemia myelofibrosis (post-PV/ET MF). In some embodiments, the subject has documented bone marrow fibrosis prior to the treatment. In some embodiments, the subject has MF-2 or higher as assessed by the European consensus grading score and grade 3 or higher by modified Bauermeister scale prior to the treatment. In some embodiments, the subject has the ECOG performance status of 1 prior to the treatment. In some embodiments, the subject has white blood cell count (10⁹/L) ranging between 5 and 120 prior to the treatment. In some embodiments, the subject has the JAK2V617F allele burden that ranges between 10-100%.

In some embodiments, a subject to receive (and who may benefit from receiving) an isoform-specific, context-independent TGFβ1 inhibitor therapy is transfusion-dependent (prior to the treatment) characterized in that the subject has a history of at least two units of red blood cell transfusions in the last month for a hemoglobin level of less than 8.5 g/dL that is not associated with clinically overt bleeding.

In some embodiments, a subject to receive (and who may benefit from receiving) an isoform-specific, context-independent TGFβ1 inhibitor therapy previously received a therapy to treat myelofibrosis. In some embodiments, the subject has been treated with one or more of therapies, including but are not limited to: AZD1480, panobinostat, EPO, IFNα, hydroxyurea, pegylated interferon, thalidomide, prednisone, and JAK2 inhibitor (e.g., Lestaurtinib, CEP-701).

In some embodiments, the patient has extramedullary hematopoiesis. In some embodiments, the extramedullary hematopoiesis is in the liver, lung, spleen, and/or lymph nodes. In some embodiments, the pharmaceutical composition of the present disclosure is administered locally to one or more of the localized sites of disease manifestation.

In some embodiments, a TGFβ inhibitor such as an isoform-specific, context-independent TGFβ1 inhibitor described herein is administered alone or in combination with a checkpoint inhibitor therapy to patients in an amount effective to treat myelofibrosis. The therapeutically effective amount is an amount sufficient to relieve one or more symptoms and/or complications of myelofibrosis in patients, including but are not limited to: excessive deposition of ECM in bone marrow stroma (fibrosis of the bone marrow), neoangiogenesis, osteosclerosis, splenomegaly, hematomegaly, anemia, bleeding, bone pain and other bone-related morbidity, extramedullary hematopoiesis, thrombocytosis, leukopenia, cachexia, infections, thrombosis and death. Thus, TGFβ inhibition therapies comprising the antibodies or antigen-binding fragments of the disclosure may achieve clinical benefits, which include, inter alia, anti-fibrotic effects and/or normalization of blood cell counts. Such therapy may prolong survival and/or reduce the need for bone marrow transplantation.

In some embodiments, the amount of TGFβ inhibitor is effective to reduce TGFβ1 expression and/or secretion (such as of megakaryocytic cells) in patients. Such inhibitor may therefore reduce TGFβ1 mRNA levels in treated patients. In some embodiments, such inhibitor reduces TGFβ1 mRNA levels in bone marrow, such as in mononuclear cells. PMF patients typically show elevated plasma TGFβ1 levels of above ˜2,500 μg/mL, e.g., above 3,000, 3,500, 4,000, 4,500, 5,000, 6,000, 7,000, 8,000, 9,000, and 10,000 μg/mL (contrast to normal ranges of ˜600-2,000 μg/mL as measured by ELISA) (see, for example, Mascaremhas et al., (Leukemia & Lymphoma, 2014, 55(2): 450-452)). Zingariello (Blood, 2013, 121(17): 3345-3363) quantified bioactive and total TGFβ1 contents in the plasma of PMF patients and control individuals. According to this reference, the median bioactive TGFβ1 in PMF patients was 43 ng/mL (ranging between 4-218 ng/mL) and total TGFβ1 was 153 ng/mL (32-1000 ng/mL), while in control counterparts, the values were 18 (0.05-144) and 52 (8-860), respectively. Thus, based on these reports, plasma TGFβ1 contents in PMF patients are elevated by several fold, e.g., 2-fold, 3-fold, 4-fold, 5-fold, etc., as compared to control or healthy plasma samples. Treatment with the inhibitor, e.g., following 4-12 cycles of administration (e.g., 2, 4, 6, 8, 10, 12 cycles) or chronic or long-term treatment, for example every 4 weeks, at dosage of 0.1-100 mg/kg, for example, 1-30 mg/kg monoclonal antibody) described herein may reduce the plasma TGFβ1 levels by at least 10% relative to the corresponding baseline (pre-treatment), e.g., at least 15%, 20%, 25%, 30%, 35%, 40%, 45%, and 50%.

Some of the therapeutic effects may be observed relatively rapidly following the commencement of the treatment, for example, after 1 week, 2 weeks, 3 weeks, 4 weeks, 5 weeks or 6 weeks. For example, the inhibitor may effectively increase the number of stem cells and/or precursor cells within the bone marrow of patients treated with the inhibitor within 1-8 weeks. These include hematopoietic stem cells and blood precursor cells. A bone marrow biopsy may be performed to assess changes in the frequencies/number of marrow cells. Correspondingly, the patient may show improved symptoms such as bone pain and fatigue.

Subjects suffering from a myeloproliferative disorder (e.g., myelofibrosis) may manifest an elevated level of white blood cell counts (e.g., leukemic). In some embodiments, the therapeutically effective amount of the TGFβ inhibitor (e.g., TGFβ1 inhibitor) is an amount that is effective to normalize blood cell counts. In some embodiments, the amount is effective to reduce total white cell counts in the subject, as compared to pre-treatment. In some embodiments, the amount is effective to reduce total platelet counts in the subject, as compared to pre-treatment. In some embodiments, the amount is effective to increase (e.g., normalize or restore) hemoglobin levels in the subject, as compared to pre-treatment. In some embodiments, the amount is effective to increase (e.g., normalize or restore) hematocrit levels in the subject, as compared to pre-treatment.

One of the morphological hallmarks of myelofibrosis is fibrosis in the bone marrow (e.g., marrow stroma), characterized in part by aberrant ECM. In some embodiments, the amount of TGFβ inhibitor (e.g., TGFβ1 inhibitor) is effective to reduce fibrosis, characterized by excessive collagen deposition, e.g., by mesenchymal stromal cells. In some embodiments, the TGFβ inhibitor is effective to reduce the number of CD41-positive cells, e.g., megakaryocytes, in treated subjects, as compared to control subjects that do not receive the treatment. In some embodiments, baseline frequencies of megakaryocytes in PMF bone marrow may range between 200-700 cells per square millimeters (mm²), and between 40-300 megakaryocites per square-millimeters (mm²) in PMF spleen, as determined with randomly chosen sections. In contrast, megakaryocyte frequencies in bone marrow and spleen of normal donors are fewer than 140 and fewer than 10, respectively. Treatment with the TGFβ inhibitor (e.g., TGFβ1 inhibitor) may reduce the number (e.g., frequencies) of megakaryocytes in bone marrow and/or spleen. In some embodiments, treatments with the inhibitor may reduce or inhibit autocrine TGFβ1 signaling in megakaryocytes. In some embodiments, treatments with the inhibitor may cause reduced levels of downstream effector signaling, such as phosphorylation of SMAD2/3, e.g., phosphorylation of SMAD2. In some embodiments, the TGFβ inhibitor (e.g., TGFβ1 inhibitor) is effective to reduce expression levels of fibrotic markers, such as those described herein. Patients with myelofibrosis may suffer from enlarged spleen. Thus, clinical effects of a therapeutic may be evaluated by monitoring changes in spleen size. Spleen size may be examined by known techniques, such as assessment of the spleen length by palpation and/or assessment of the spleen volume by ultrasound. In some embodiments, the subject to be treated with an isoform-specific, context-independent inhibitor of TGFβ1 has a baseline spleen length (prior to the treatment) of 5 cm or greater, e.g., ranging between 5 and 30 cm as assessed by palpation. In some embodiments, the subject to be treated with an isoform-specific, context-independent inhibitor of TGFβ1 has a baseline spleen volume (prior to the treatment) of 300 mL or greater, e.g., ranging between 300-1500 mL, as assessed by ultrasound. Treatment with the inhibitor, e.g., following 4-12 cycles of administration (e.g., 2, 4, 6, 8, 10, 12 cycles), for example every 4 weeks, at dosage of 0.1-30 mg/kg monoclonal antibody) described herein may reduce spleen size in the subject. In some embodiments, the effective amount of the inhibitor is sufficient to reduce spleen size in a patient population that receives the inhibitor treatment by at least 10%, 20%, 30%, 35%, 40%, 50%, and 60%, relative to corresponding baseline values. For example, the treatment is effective to achieve a ≥35% reduction in spleen volume from baseline in 12-24 weeks as measured by MRI or CT scan, as compared to placebo control. In some embodiments, the treatment is effective to achieve a ≥35% reduction in spleen volume from baseline in 24-48 weeks as measured by MRI or CT scan, as compare to best available therapy control. Best available therapy may include hydroxyurea, glucocorticoids, as well as no medication, anagrelide, epoetin alfa, thalidomide, lenalidomide, mercaptopurine, thioguanine, danazol, peginterferon alfa-2a, interferon-α, melphalan, acetylsalicylic acid, cytarabine, and colchicine.

In some embodiments, a patient population treated with a TGFβ inhibitor such as an isoform-specific, context-independent TGFβ1 inhibitor described herein shows a statistically improved treatment response as assessed by, for example, International Working Group for Myelofibrosis Research and Treatment (IWG-MRT) criteria, degree of change in bone marrow fibrosis grade measured by the modified Bauermeister scale and European consensus grading system after treatment (e.g., 4, 6, 8, or 12 cycles), symptom response using the Myeloproliferative Neoplasm Symptom Assessment Form (MPN-SAF).

In some embodiments, the treatment with an isoform-specific, context-independent TGFβ1 inhibitor such as those described herein, achieves a statistically improved treatment response as assessed by, for example, modified Myelofibrosis Symptom Assessment Form (MFSAF), in which symptoms are measured by the MFSAF tool (such as v2.0), a daukt diary capturing the debilitating symptoms of myelofibrosis (abdominal discomfort, early satiety, pain under left ribs, pruritus, night sweats, and bone/muscle pain) using a scale of 0 to 10, where 0 is absent and 10 is the worst imaginable. In some embodiments, the treatment is effective to achieve a 50%≥reduction in total MFSAF score from the baseline in, for example, 12-24 weeks. In some embodiments, a significant fraction of patients who receive the therapy achieves a ≥50% improvement in Total Symptom Score, as compared to patients taking placebo. For example, the fraction of the patient pool to achieve ≥50% improvement may be over 40%, 50%, 55%, 60%, 65%, 70%, 75% or 80%.

In some embodiments, the therapeutically effective amount of the inhibitor is an amount sufficient to attain clinical improvement as assessed by an anemia response. For example, an improved anemia response may include longer durations of transfusion-independence, e.g., 8 weeks or longer, following the treatment of 4-12 cycles, e.g., 6 cycles.

In some embodiments, the therapeutically effective amount of the inhibitor is an amount sufficient to maintain stable disease for a duration of time, e.g., 6 weeks, 8 weeks, 12 weeks, six months, etc. In some embodiments, progression of the disease may be evaluated by changes in overall bone marrow cellularity, the degree of reticulin or collagen fibrosis, and/or a change in JAK2V617F allele burden.

In some embodiments, a patient population treated with an isoform-specific, context-independent TGFβ1 inhibitor such as those described herein, shows statistically improved (prolonged) survival, as compared to a control population that does not receive the treatment. For example, in control groups, median survival of PMF patients is approximately six years (approximately 16 months in high-risk patients), and fewer than 20% of the patients are expected to survive 10 years or longer post-diagnosis. Treatment with the isoform-specific, context-independent TGFβ1 inhibitor such as those described herein, may prolong the survival time by, at least 6 months, 12 months, 18 months, 24 months, 30 months, 36 months, or 48 months. In some embodiments, the treatment is effective to achieve improved overall survival at 26 weeks, 52 weeks, 78 weeks, 104 weeks, 130 weeks, 144 weeks, or 156 weeks, as compared to patients who receive placebo.

Clinical benefits of the therapy, such as those exemplified above, may be seen in patients with or without new onset anemia.

One of the advantageous features of the isoform-specific, context-independent TGFβ1 inhibitors is that they maintain improved safety profiles enabled by isoform selectivity, as compared to conventional TGFβ antagonists that lack the selectivity. Therefore, it is anticipated that treatment with an isoform-specific, context-independent inhibitor, such as those described herein, may reduce adverse events in a patient population, in comparison to equivalent patient populations treated with conventional TGFβ antagonists, with respect to the frequency and/or severity of such events. Thus, the isoform-specific, context-independent TGFβ1 inhibitors may provide a greater therapeutic window as to dosage and/or duration of treatment.

Adverse events may be graded by art-recognized suitable methods, such as Common Terminology Criteria for Adverse Events (CTCAE) version 4. Previously reported adverse events in human patients who received TGFβ antagonists, such as GC1008, include: leukocytosis (grade 3), fatigue (grade 3), hypoxia (grade 3), asystole (grade 5), leukopenia (grade 1), recurrent, transient, tender erythematous, nodular skin lesions, suppurative dermatitis, and herpes zoster.

The TGFβ1 inhibitor therapy may cause less frequent and/or less severe adverse events (side effects) as compared to JAK inhibitor therapy in myelofibrosis patients, with respect to, for example, anemia, thrombocytopenia, neutropenia, hypercholesterolemia, elevated alanine transaminase (ALT), elevated aspartate transaminase (AST), bruising, dizziness, and headache, thus offering a safer treatment option.

It is contemplated that inhibitors of TGFβ signaling may be used in conjunction with one or more therapeutic agents to treat myelofibrosis as a combination (e.g., “add-on”) therapy. In some embodiments, the TGF3 inhibitor is an inhibitor of TGFβ activation, e.g., TGFβ1 activation, e.g., Ab6, which is administered in combination with one or more checkpoint inhibitors disclosed herein to a patient suffering from myelofibrosis. In some embodiments, the TGFβ inhibitor such as Ab6 is administered to a patient suffering from myelofibrosis who has received or is a candidate for receiving a JAK1 inhibitor, JAK2 inhibitor or JAK1/JAK2 inhibitor. In some embodiments, such patients are responsive to the JAK1 inhibitor, JAK2 inhibitor or JAK1/JAK2 inhibitor therapy, while in other embodiments such patients are poorly responsive or not responsive to the JAK1 inhibitor, JAK2 inhibitor or JAK1/JAK2 inhibitor therapy. In some embodiments, use of a TGFβ inhibitor such as an isoform-specific inhibitor of TGFβ1 described herein may render those who are poorly responsive or not responsive to the JAK1 inhibitor, JAK2 inhibitor or JAK1/JAK2 inhibitor therapy more responsive. In some embodiments, use of a TGFβ inhibitor such as an isoform-specific inhibitor of TGFβ1 described herein may allow reduced dosage of the JAK1 inhibitor, JAK2 inhibitor or JAK1/JAK2 inhibitor which still produces equivalent or meaningful clinical efficacy or benefits in patients but with fewer or lesser degrees of drug-related toxicities or adverse events (such as those listed above). In some embodiments, treatment with the inhibitor of TGFβ activation described herein used in conjunction with JAK1 inhibitor, JAK2 inhibitor or JAK1/JAK2 inhibitor therapy may produce synergistic or additive therapeutic effects in patients. In some embodiments, treatment with the inhibitor of TGFβ activation described herein may boost the benefits of JAK1 inhibitor, JAK2 inhibitor or JAK1/JAK2 inhibitor or other therapy given to treat myelofibrosis. In some embodiments, patients may additionally receive a therapeutic to address anemia associated with myelofibrosis.

In some embodiments, a TGFβ inhibitor described herein, such as a TGFβ31-selective inhibitor described herein (e.g., Ab6), may be used to provide therapeutic benefit in conjunction with a checkpoint inhibitor therapy for the treatment of myelofibrosis. Primary cells isolated from patients with JAK2 mutation exhibit higher PD-L1 expression as compared to primary cells from healthy donors. This indicates that constitutive activation of the JAK2/STAT pathway in megakaryocytes and platelets may contribute to immune escape via PD-L1-mediated reduction of T cell activation, metabolic activity, and cell cycle progression of T cells (Prestipino et al., Sci Transl Med 2018; 10(429)). Additionally, activation of the TGFβ signaling pathway has also been shown to increase PD-1 expression on cytotoxic T cells and decrease sensitivity to PD-1/PD-L1-mediated checkpoint blockade (Chen et al., Int J Cancer 2018; 143:2561). Without being bound by theory, these findings, along with low response rates to checkpoint inhibitor therapy (e.g., anti-PD-1 therapy) observed in myelofibrosis patients, provide support for the potential importance of TGFβ signaling in mediating clinical resistance to checkpoint inhibitor therapy.

In some embodiments, a TGFβ inhibitor such as a TGFβ1 inhibitor (e.g., Ab6) may be used in conjunction with a BMP antagonist (e.g., a BMP6 inhibitor, e.g., a RGMc inhibitor) for treating anemia in a patient with a myeloproliferative disorder such as myelofibrosis. Without wishing to be bound by theory, it is contemplated that TGFβ1 inhibitors (e.g., Ab6) may be helpful for promoting hematopoiesis, while BMP antagonists (e.g., BMP6 inhibitors, e.g., RGMc inhibitors) may reduce iron deficiency (such as a deficiency arising from a cancer and/or chemotherapy). In some embodiments, a treatment comprising a TGFβ1 inhibitor (e.g., Ab6) and a BMP antagonist (e.g., a BMP6 inhibitor, e.g., a RGMc inhibitor) may be administered at a therapeutically effective amount that is sufficient to relieve one or more anemia-related symptom and/or complication. In some embodiments, a treatment comprising a TGFβ1 inhibitor (e.g., Ab6) and a BMP antagonist (e.g., a BMP6 inhibitor, e.g., a RGMc inhibitor) may be administered at a therapeutically effective amount to increase red blood cell production and/or reduce iron restriction, in a patient with a myeloproliferative disorder (e.g., myelofibrosis). In some embodiments, the treatment for anemia further comprises administering one or more JAK inhibitor (e.g., Jaki/2 inhibitor, Jaki inhibitor, and/or Jak2 inhibitor). In some embodiments, an improved anemia response may include a longer duration of transfusion-independence, e.g., 8 weeks or longer, e.g., following treatment for 4-12 cycles, e.g., 6 cycles. In some embodiments, the treatment further includes one or more checkpoint inhibitors such as anti-PD1 antibodies, anti-PD-L1 antibodies, and/or anti-CTLA-4 antibodies.

Accordingly, the present disclosure provides a TGFβ inhibitor (e.g., TGFβ31-selective inhibitor such as Ab6) for use in the treatment of a myeloproliferative disorder such as primary myelofibrosis in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Conditions Involving MHC Downregulation or Mutation

TGFβ-related indications may also include conditions in which major histocompatibility complex (MHC) class I is deleted or deficient (e.g., downregulated). Such conditions include genetic disorders in which one or more components of the MHC-mediated signaling is impaired, as well as conditions in which MHC expression is altered by other factors, such as cancer, infections, fibrosis, and medications.

For example, MHC I downregulation in tumor is associated with tumor escape from immune surveillance. Indeed, immune escape strategies aimed to avoid T-cell recognition, including the loss of tumor MHC class I expression, are commonly found in malignant cells. Tumor immune escape has been observed to have a negative effect on the clinical outcome of cancer immunotherapy, including treatment with antibodies blocking immune checkpoint molecules (reviewed in, for example: Garrido et al., (2017) Curr Opin Immunol 39: 44-51. “The urgent need to recover MHC class I in cancers for effective immunotherapy”, incorporated by reference herein). Thus, the isoform-selective, context-independent TGFβ1 inhibitors encompassed by the present disclosure may be administered either as a monotherapy or in conjunction with another therapy (such as checkpoint inhibitor, chemotherapy, radiation therapy (such as a radiotherapeutic agent), etc.) to unleash or boost anti-cancer immunity and/or enhance responsiveness to or effectiveness of another therapy.

In some embodiments, MHC downregulation is associated with acquired resistance to a cancer therapy, such as CBT. It is contemplated that the isoform-selective inhibitors of TGFβ1 may be used to treat patients who are primary responders of a cancer therapy such as CBT, to reduce the probability of developing acquired resistance. Thus, among those treated with the TGFβ1 inhibitor, who are primary responders of cancer therapy, occurrence of secondary or acquired resistance to the cancer therapy over time may be reduced.

Downregulation of MHC class I proteins are also associated with certain infectious diseases, including viral infections such as HIV. See for example, Cohen et al., (1999) Immunity 10(6): 661-671. “The selective downregulation of class I major histocompatibility complex proteins by HIV-1 protects HIV-infected cells from NK Cells”, incorporated herein by reference. Thus, the isoform-selective, context-independent TGFβ1 inhibitors encompassed by the present disclosure may be administered either as a monotherapy or in conjunction with another therapy (such as anti-viral therapy, protease inhibitor therapy, etc.) to unleash or boost host immunity and/or enhance responsiveness to or effectiveness of another therapy.

The present disclosure provides a TGFβ inhibitor (e.g., TGFβ1-selective inhibitor such as Ab6) for use in the treatment of a TGFβ-related disorder involving MHC downregulation or mutation (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Conditions Involving Stem Cell Self-Renewal, Tissue Regeneration and Stem Cell Repopulation

Evidence suggests that the TGFβ pathway plays a role in regulating the homeostasis of various stem cell populations and their differentiation/repopulation within a tissue. During homeostasis, tissue-specific stem cells are held predominantly quiescent but are triggered to enter cell cycle upon certain stress. TGFβ1 is thought to function as a “break” during the process that tightly regulates stem cell differentiation and reconstitution, and the stress that triggers cell cycle entry coincides with TGFβ1 inhibition that removes the “break.” Thus, it is contemplated that isoform-selective inhibitors of TGFβ1, such as those described herein, may be used to skew or correct cell cycle and G0 entry decision of stem cells/progenitor cells within a particular tissue.

Accordingly, the inventors of the present disclosure contemplate the use of isoform-selective TGFβ1 inhibitors in conditions in which: i) stem cell/progenitor cell differentiation/reconstitution is halted or perturbed due to a disease or induced as a side effect of a therapy/mediation; ii) patients are on a therapy or mediation that causes healthy cells to be killed or depleted; iii) patients may benefit from increased stem cell/progenitor cell differentiation/reconstitution; iv) disease is associated with abnormal stem cell differentiation or reconstitution.

In self-renewing tissues, such as bone marrow (blood cell production) and the epidermis, the balance between proliferation and differentiation processes is tightly regulated to ensure the maintenance of the stem cell population during lifetime. Reviewed by D'Arcangel et al., (2017) Int. J Mol Sci. 18(7): 1591. TGFβ1 acts as a potent negative regulator of the cell cycle and tumor suppressor in part through induction of cyclin-dependent kinase inhibitors, p15/INK4b, p21 and p57. Evidence suggests that TGFβ1 contributes to the induction of p16/INK4a and p19/ARF to mediate growth arrest and senescence in certain cell types. Accordingly, in some embodiments, an isoform-selective inhibitor of TGFβ1 activation, such as those described herein, is used to regulate p16/INK4a-dependent cellular senescence and stem cell dynamics in various stem cell populations.

For example, mesenchymal stromal/stem cells (MSCs) are a small population of stromal cells present in most adult connective tissues, such as bone marrow, fat tissue, and umbilical cord blood. MSCs are maintained in a relative state of quiescence in vivo but, in response to a variety of physiological and pathological stimuli, are capable of proliferating then differentiating into osteoblasts, chondrocytes, adipocytes, or other mesoderm-type lineages like smooth muscle cells (SMCs) and cardiomyocytes. Multiple signaling networks orchestrate MSCs differentiating into functional mesenchymal lineages, among which TGF-β1 has emerged as a key player (reviewed for example by Zhao & Hantash (2011. Vitam Horm 87:127-41).

Similarly, hematopoietic stem cells are required for lifelong blood cell production; to prevent exhaustion, the majority of hematopoietic stem cells remain quiescent during steady-state hematopoiesis. During hematologic stress, however, these cells are rapidly recruited into cell cycle and undergo extensive self-renewal and differentiation to meet increased hematopoietic demands. TGFβ1 may work as the “switch” to control the quiescence-repopulation transition/balance.

Thus, the isoform-selective inhibitors of TGFβ1 can be used in the treatment of conditions involving hematopoietic stem cell defects and bone marrow failure. In some embodiments, depletion or impairment of the hematopoietic stem cell reservoir leads to hematopoietic failure or hematologic malignancies. In some embodiments, such conditions are DNA repair disorder characterized by progressive bone marrow failure. In some embodiments, such condition is caused by stem and progenitor cell attrition. In some embodiments, such conditions are associated with anemia. In some embodiments, such condition is Fanconi Anemia (FA). In some embodiments, such conditions are characterized by hyperactive TGFβ pathway that suppresses the survival of certain cell types upon DNA damage. Thus, it is contemplated that the isoform-selective inhibitors of TGFβ1 can be used for rescuing proliferation defects of FA hematopoietic stem cells and/or bone marrow failure in subjects with FA. See, for example, Zhang et al., (2016), Cell Stem Cell, 18: 668-681, “TGF-β inhibition rescues hematopoietic stem cell defects and bone marrow failure in Fanconi Anemia.”

The present disclosure provides a TGFβ inhibitor (e.g., TGFβ1-selective inhibitor such as Ab6) for use in the treatment of a TGFβ-related disorder involving stem cell self-renewal, tissue regeneration and/or stem cell repopulation (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

### Conditions Involving Treatment-Induced Hematopoietic Dysregulation

It is recognized that certain drugs which are designed to treat various disease conditions, often induce or exacerbate anemia in the patient being treated (e.g., treatment- or drug-induced anemia, such as chemotherapy-induced anemia and radiation therapy-induced anemia). In some embodiments, the patient is treated with a myelosuppressive drug that may cause side effects that include anemia. Such patient may benefit from pharmacological TGFβ1 inhibition in order to boost hematopoiesis. In some embodiments, the TGFβ1 inhibitor may promote hematopoiesis in patients by preventing entry into a quiescent state. In some embodiments, the patient may receive a G-CSF therapy (e.g., Filgrastim).

Accordingly, the disclosure includes the use of an isoform-selective inhibitor of TGFβ1, such as those disclosed herein, to be administered to patients who receive myelosuppressive therapy (e.g., therapy with side effects including myelosuppressive effects). Examples of myelosuppressive therapies include but are not limited to: peginterferon alfa-2a, interferon alfa-n3, peginterferon alfa-2b, aldesleukin, gemtuzumab ozogamicin, interferon alfacon-1, rituximab, ibritumomabtiuxetan, tositumomab, alemtuzumab, bevacizumab, L-Phenylalanine, bortezomib, cladribine, carmustine, amsacrine, chlorambucil, raltitrexed, mitomycin, bexarotene, vindesine, floxuridine, tioguanine, vinorelbine, dexrazoxane, sorafenib, streptozocin, gemcitabine, teniposide, epirubicin, chloramphenicol, lenalidomide, altretamine, zidovudine, cisplatin, oxaliplatin, cyclophosphamide, fluorouracil, propylthiouracil, pentostatin, methotrexate, carbamazepine, vinblastine, linezolid, imatinib, clofarabine, pemetrexed, daunorubicin, irinotecan, methimazole, etoposide, dacarbazine, temozolomide, tacrolimus, sirolimus, mechlorethamine, azacitidine, carboplatin, dactinomycin, cytarabine, doxorubicin, hydroxyurea, busulfan, topotecan, mercaptopurine, thalidomide, melphalan, fludarabine, flucytosine, capecitabine, procarbazine, arsenic trioxide, idarubicin, ifosfamide, mitoxantrone, lomustine, paclitaxel, docetaxel, dasatinib, decitabine, nelarabine, everolimus, vorinostat, thiotepa, ixabepilone, nilotinib, belinostat, trabectedin, trastuzumab emtansine, temsirolimus, bosutinib, bendamustine, cabazitaxel, eribulin, ruxolitinib, carfilzomib, tofacitinib, ponatinib, pomalidomide, obinutuzumab, tedizolid phosphate, blinatumomab, ibrutinib, palbociclib, olaparib, dinutuximab, and colchicine.

Additional TGFβ-related indications may include any of those disclosed in US Pub. No. 2013/0122007, U.S. Pat. No. 8,415,459 or International Pub. No. WO 2011/151432, the contents of each of which are herein incorporated by reference in their entirety.

In certain embodiments, antibodies, antigen binding portions thereof, and compositions of the disclosure may be used to treat a wide variety of diseases, disorders and/or conditions associated with TGFβ1 signaling. In some embodiment, target tissues/cells preferentially express the TGFβ1 isoform over the other isoforms. Thus, the disclosure includes methods for treating such a condition associated with TGFβ1 expression (e.g., dysregulation of TGFβ1 signaling and/or upregulation of TGFβ1 expression) using a pharmaceutical composition that comprises an antibody or antigen-binding portion thereof described herein.

In some embodiments, the disease involves TGFβ1 associated with (e.g., presented on or deposited from) multiple cellular sources. In some embodiments, such disease involves both an immune component and an ECM component of TGFβ1 function. In some embodiments, such disease involves: i) dysregulation of the ECM (e.g., overproduction/deposition of ECM components such as collagens and proteases; altered stiffness of the ECM substrate; abnormal or pathological activation or differentiation of fibroblasts, such as myofibroblasts, fibrocytes and CAFs); ii) immune suppression due to increased Tregs and/or suppressed effector T cells (Teff), e.g., elevated ratios of Treg/Teff; increased leukocyte infiltrate (e.g., macrophage and MDSCs) that causes suppression of CD4 and/or CD8 T cells; and/or iii) abnormal or pathological activation, differentiation, and/or recruitment of myeloid cells, such as macrophages (e.g., bone marrow-derived monocytic/macrophages and tissue resident macrophages), monocytes, myeloid-derived suppresser cells (MDSCs), neutrophils, dendritic cells, and NK cells.

In some embodiments, the condition involves TGFβ1 presented by more than one types of presenting molecules (e.g., two or more of: GARP, LRRC33, LTBP1 and/or LTBP3). In some embodiments, an affected tissues/organs/cells that include TGFβ1 from multiple cellular sources. To give but one example, a solid tumor (which may also include a proliferative disease involving the bone marrow, e.g., myelofibrosis and multiple myeloma) may include TGFβ1 from multiple sources, such as the cancer cells, stromal cells, surrounding healthy cells, and/or infiltrating immune cells (e.g., CD45+ leukocytes), involving different types of presenting molecules. Relevant immune cells include but are not limited to myeloid cells and lymphoid cells, for example, neutrophils, eosinophils, basophils, lymphocytes (e.g., B cells, T cells, and NK cells), and monocytes. Context-independent inhibitors of TGFβ1 may be useful for treating such conditions.

In some embodiments, hematopoietic dysregulation associated with cancer may cause anemia. The TGFβ therapy may further include a BMP6 inhibitor, such as a RGMc inhibitor. In some embodiments, cancer-associated anemia may be caused by the disease itself; while in some embodiments the anemia may be caused by cancer therapy (such as chemotherapy).

The present disclosure provides a TGFβ inhibitor (e.g., TGFβ1-selective inhibitor such as Ab6) for use in the treatment of a TGFβ-related disorder involving treatment-induced hematopoietic dysregulation (e.g., as described herein) in a patient, wherein the treatment comprises administration of a composition comprising the TGFβ inhibitor (e.g., TGFβ1 inhibitor) which has been selected, at least in part, on the basis of its immune safety profile. A suitable immune safety profile of the TGFβ inhibitor is characterized in that i) it does not trigger unacceptable levels of cytokine release (e.g., within 2.5-fold of control); ii) it does not promote unacceptable levels of platelet aggregation; or both in field-accepted cell-based assay(s) and/or in in vivo assay(s) (such as those described herein).

Non-limiting examples of conditions or disorders that may be treated with isoform-specific context-independent inhibitors of TGFβ1, such as antibodies or fragments thereof described herein, are provided below.

### Treatment Regimen, Administration

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation ortopical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a TGFβ-related indication, such as those noted above. Asubject having a TGFβ-related indication can be identified by routine medical examination, e.g., laboratory tests, organ functional tests, CT scans, or ultrasounds. A subject suspected of having any of such indication might show one or more symptoms of the indication. A subject at risk for the indication can be a subject having one or more of the risk factors for that indication.

As used herein, the terms “effective amount” and “effective dose” refer to any amount or dose of a compound or composition that is sufficient to fulfill its intended purpose(s), i.e., a desired biological or medicinal response in a tissue or subject at an acceptable benefit/risk ratio. For example, in certain embodiments of the present invention, the intended purpose may be to inhibit TGFβ-1 activation in vivo, to achieve clinically meaningful outcome associated with the TGFβ-1 inhibition. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a TGFβ-related indication. Alternatively, sustained continuous release formulations of an antibody that specifically binds a GARP-TGFβ1 complex, a LTBP1-TGFβ1 complex, a LTBP3-TGFβ1 complex, and/or a LRRC33-TGFβ1 complex may be appropriate. Various formulations and devices for achieving sustained release would be apparent to the skilled artisan and are within the scope of this disclosure.

In one example, dosages for an antibody as described herein may be determined empirically in individuals who have been given one or more administration(s) of the antibody. Individuals are given incremental dosages of the antagonist. To assess efficacy, an indicator of the TGFβ-related indication can be followed. For example, methods for measuring for myofiber damage, myofiber repair, inflammation levels in muscle, and/or fibrosis levels in muscle are well known to one of ordinary skill in the art.

The present disclosure encompasses the recognition that agents capable of modulating the activation step of TGFβs in an isoform-specific manner may provide improved safety profiles when used as a medicament. Accordingly, the disclosure includes antibodies and antigen-binding fragments thereof that specifically bind and inhibit activation of TGFβ1, but not TGFβ2 or TGFβ3, thereby conferring specific inhibition of the TGFβ1 signaling in vivo while minimizing unwanted side effects from affecting TGFβ2 and/or TGFβ3 signaling.

In some embodiments, the antibodies, or antigen binding portions thereof, as described herein, are not toxic when administered to a subject. In some embodiments, the antibodies, or antigen binding portions thereof, as described herein, exhibit reduced toxicity when administered to a subject as compared to an antibody that specifically binds to both TGFβ1 and TGFβ2. In some embodiments, the antibodies, or antigen binding portions thereof, as described herein, exhibit reduced toxicity when administered to a subject as compared to an antibody that specifically binds to both TGFβ1 and TGFβ3. In some embodiments, the antibodies, or antigen binding portions thereof, as described herein, exhibit reduced toxicity when administered to a subject as compared to an antibody that specifically binds to TGFβ1, TGFβ2 and TGFβ3.

Generally, for administration of any of the antibodies described herein, an initial candidate dosage can be about 1-20 mg/kg per administration, with dosing frequency of, e.g., once weekly (QW), once every 2 weeks (Q2W), once every 3 weeks (Q3W), once every 4 weeks (Q4W), monthly, etc. For example, patients may receive an injection of about 1-10 mg/kg per 1 week, per 2 weeks, per 3 weeks, or per 4 weeks, etc., in an amount effective to treat a disease (e.g., cancer) wherein the amount is well-tolerated (within acceptable toxicities or adverse events).

For the purpose of the present disclosure, a typical dosage (per administration, such as an injection and infusion) might range from about 0.1 mg/kg to 30 mg/kg, depending on the factors mentioned above. For repeated administrations over several days or longer, depending on the condition, the treatment is sustained until a desired suppression of symptoms occurs or until sufficient therapeutic levels are achieved to alleviate a TGFβ-related indication, or a symptom thereof. For example, suitable effective dosage for Ab6 may be between 1 mg/kg and 30 mg/kg, (e.g., 1-10 mg/kg, 1-15 mg/kg, 3-20 mg/kg, 5-30 mg/kg, etc.) dosed twice a week, once a week, every two weeks, every 4 weeks or once a month. Suitable effective dose for Ab6 includes, about 1 mg/kg, about 3 mg/kg, about 5 mg/kg, about 10 mg/kg, for example, dosed weekly.

An exemplary dosing regimen comprises administering an initial dose, followed by one or more of maintenance doses. For example, an initial dose may be between about 1 and 30 mg/kg, for instance, once a week or twice a week. Thereafter, maintenance dose(s) may follow, for example, between about 0.1 and 20 mg/kg, for instance, once a week, every other week, once a month, etc. However, other dosage regimens may be useful, depending on the pattern of pharmacokinetic decay that the practitioner wishes to achieve. Pharmacokinetics experiments have shown that the serum concentration of an antibody disclosed herein (e.g., Ab3) remains stable for at least 7 days after administration to a preclinical animal model (e.g., a mouse model). Without wishing to be bound by any particular theory, this stability post-administration may be advantageous since the antibody may be administered less frequently while maintaining a clinically effective serum concentration in the subject to whom the antibody is administered (e.g., a human subject). In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 1 mg/kg to 30 mg/kg, or from 80 mg to 3000 mg, e.g., 30, 50, 100, 500, 1000, 2000, or 3000 mg, may be administered. The particular dosage regimen, e.g., dose, timing, and repetition, will depend on the particular individual and that individual's medical history, as well as the properties of the individual agents (such as the half-life of the agent, and other relevant considerations).

Serum concentrations of the TGF3 inhibitor antibody that are therapeutically effective to treat a TGFβ1-related indication in accordance with the present disclosure may be at least about 10 μg/mL, e.g., between about 10 μg/mL and 1.0 mg/mL. In some embodiments, effective amounts of the antibody as measured by serum concentrations are about 20-400 μg/mL. In some embodiments, effective amounts of the antibody as measured by serum concentrations are about 100-800 μg/mL. In some embodiments, effective amounts of the antibody as measured by serum concentrations are at least about 20 μg/mL, e.g., at least about 50 μg/mL, 100 μg/mL, 150 μg/mL, or 200 μg/mL. As shown previously, a single dose of Ab6 administered intravenously at 1, 3, 10, 30, or 37.5 mg/kg resulted in Cₘₐₓ values of about 25 μg/mL to about 900 μg/mL. Furthermore, in non-human primates, there were no observed toxicities (for example: no cardiotoxicities, hyperplasia and inflammation, dental and gingival findings) associated with Ab6 after maintaining serum concentration levels of about 2,000-3,000 μg/mL for at least 8 weeks (data provided in PCT/US2019/041373 at Example 12). Therefore, about 10-100 fold therapeutic window may be achieved.

For the purpose of the present disclosure, the appropriate dosage of an antibody that specifically binds a GARP-TGFβ1 complex, a LTBP1-TGFβ1 complex, a LTBP3-TGFβ1 complex, and/or a LRRC33-TGFβ1 complex will depend on the specific antibody (or compositions thereof) employed, the type and severity of the indication, whether the antibody is administered for preventive or therapeutic purposes, previous therapy, the patient's clinical history and response to the antagonist, and the discretion of the attending physician. In some embodiments, a clinician will administer an antibody that specifically binds a GARP-TGFβ1 complex, a LTBP1-TGFβ1 complex, a LTBP3-TGFβ1 complex, and/or a LRRC33-TGFβ1 complex, until a dosage is reached that achieves the desired result. Administration of an antibody that specifically binds a GARP-TGFβ1 complex, a LTBP1-TGFβ1 complex, a LTBP3-TGFβ1 complex, and/or a LRRC33-TGFβ1 complex can be continuous or intermittent, depending, for example, upon the recipient's physiological condition, whether the purpose of the administration is therapeutic or prophylactic, and other factors known to skilled practitioners. The administration of antibody that specifically binds a GARP-TGFβ1 complex, a LTBP1-TGFβ1 complex, a LTBP3-TGFβ1 complex, and/or a LRRC33-TGFβ1 complex may be essentially continuous over a preselected period of time or may be in a series of spaced dose, e.g., either before, during, or after developing a TGFβ-related indication.

In order to minimize potential risk and adverse events associated with TGFβ inhibition, a TGFβ inhibitor such as any one of the antibodies disclosed herein may be administered intermittently. For instance, the TGFβ inhibitor may be administered on an “as needed” basis in a therapeutically effective amount sufficient to achieve and maintain clinical benefit (e.g., reduction of tumor volume and/or reversal or reduction of immunosuppression). In some embodiments, administration of a TGFβ inhibitor such as any one of the antibodies disclosed herein (e.g., a TGFβ1 inhibitor, e.g., Ab6) may be used in combination with a method of determining or monitoring therapeutic efficacy (e.g., measuring of circulating MDSCs). In some embodiments, the TGFβ inhibitor is administered in patients only when clinical benefit from additional doses of the TGFβ inhibitor is determined, e.g., when an elevation in circulating MDSCs is detected.

As used herein, the term “treating” refers to the application or administration of a composition including one or more active agents to a subject, who has a TGFβ-related indication, a symptom of the indication, or a predisposition toward the indication, with the purpose to cure, heal, alleviate, relieve, alter, remedy, ameliorate, improve, or affect the indication, the symptom of the indication, or the predisposition toward the indication.

Alleviating a TGFβ-related indication with an antibody that specifically binds a GARP-TGFβ1 complex, a LTBP1-TGFβ1 complex, a LTBP3-TGFβ1 complex, and/or a LRRC33-TGFβ1 complex includes delaying the development or progression of the indication, or reducing indication's severity. Alleviating the indication does not necessarily require curative results. As used therein, “delaying” the development of an indication associated with a TGFβ-related indication means to defer, hinder, slow, retard, stabilize, and/or postpone progression of the indication. This delay can be of varying lengths of time, depending on the history of the indication and/or individuals being treated. A method that “delays” or alleviates the development of an indication, or delays the onset of the indication, is a method that reduces probability of developing one or more symptoms of the indication in a given time frame and/or reduces extent of the symptoms in a given time frame, when compared to not using the method. Such comparisons are typically based on clinical studies, using a number of subjects sufficient to give a statistically significant result.

### Diagnostics, Patient Selection, Monitoring

Therapeutic methods that include TGFβ inhibition therapy may comprise diagnosis of a TGFβ indication and/or selection of patients likely to respond to such therapy. Additionally, patients who receive the TGFβ inhibitor may be monitored for therapeutic effects of the treatment, which typically involves measuring one or more suitable parameters, which are indicative of the condition, and which can be measured (e.g., assayed) before and after the treatment, and evaluating treatment-related changes in the parameters. For example, such parameters may include levels of biomarkers present in biological samples collected from the patients. Biomarkers may be RNA-based, protein-based, cell-based, and/or tissue-based. For example, genes that are overexpressed in certain disease conditions may serve as the biomarkers to diagnose and/or monitor the disease or response to the therapy. Cell-surface proteins of disease-associated cell populations may serve as biomarkers. Such methods may include the direct measurements of disease parameters indicative of the extent of the particular disease, such as tumor size/volume. Any suitable sampling methods may be employed, such as serum/blood samples, biopsies, and imaging. The biopsy may include tissue biopsies (such as tumor biopsy, e.g., core needle biopsy) and liquid biopsies.

While biopsies have traditionally been the standard for diagnosing and monitoring various diseases, such as proliferative disorders (e.g., cancer), less invasive alternatives may be preferred. For example, many non-invasive in vivo imaging techniques may be used to diagnose, monitor, and select patients for treatment. Thus, the disclosure includes the use of in vivo imaging techniques to diagnose and/or monitor disease in a patient or subject. In some embodiments, the patient or subject is receiving an isoform-specific TGFβ1 inhibitor as described herein. In other embodiments, an in vivo imaging technique may be used to select patients for treatment with an isoform-specific TGFβ1 inhibitor. In some embodiments, such techniques may be used to determine if or how patients respond to a therapy, e.g., TGFβ1 inhibition therapy.

Exemplary in vivo imaging techniques used for the methods include, but are not limited to X-ray radiography, magnetic resonance imaging (MRI), medical ultrasonography or ultrasound, endoscopy, elastography, tactile imaging, thermography, medical photography. Other imaging techniques include nuclear medicine functional imaging, e.g., positron emission tomography (PET) and Single-photon emission computed tomography (SPECT). Methods for conducting these techniques and analyzing the results are known in the art.

Non-invasive imaging techniques commonly used to diagnose and monitor cancer include, but are not limited to: magnetic resonance imaging (MRI), computed tomography (CT), ultrasound, positron emission tomography (PET), single-photon emission computed tomography (SPECT), fluorescence reflectance imaging (FRI), and fluorescence mediated tomography (FMT). Hybrid imaging platforms may also be used to diagnose and monitor cancer. For example, hybrid techniques include, but are not limited to: PET-CT, FMT-CT, FMT-MRI, and PET-MRI. Dynamic contrast enhanced MRI (DCE-MRI) is another imaging technique commonly used to detect breast cancers. Methods for conducting these techniques and analyzing the results are known in the art.

More recently, non-invasive imaging methods are being developed which will allow the detection of cells of interest (e.g., cytotoxic T cells, macrophages, and cancer cells) in vivo. See, for example, www.imaginab.com/technology/; Tavare et al., (2014) PNAS, 111(3): 1108-1113; Tavare et al., (2015) J NucI Med 56(8): 1258-1264; Rashidian et al., (2017) J Exp Med 214(8): 2243-2255; Beckford Vera et al., (2018) PLoS ONE 13(3): e0193832; and Tavare et al., (2015) Cancer Res 76(1): 73-82, each of which is incorporated herein by reference. So-called “T-cell tracking” is aimed to detect and localize anti-tumor effector T-cells in vivo. This may provide useful insights into understanding the immunosuppressive phenotype of solid tumors. Tumors that are well-infiltrated with cytotoxic T cells (“inflamed” or “hot” tumors) are likely to respond to cancer therapies such as checkpoint blockade therapy (CBT). On the other hand, tumors with immunosuppressive phenotypes tend to have poor T-cell infiltration even when there is an anti-tumor immune response. These so-called “immune excluded” tumors likely fail to respond to cancer therapies such as CBT. T-cell tracking techniques may reveal these different phenotypes and provide information to guide in therapeutic approach that would likely benefit the patients. For example, patients with an “immune excluded” tumor are likely to benefit from a TGFβ inhibitor therapy such as a TGFβ1 inhibitor therapy to help reverse the immunosuppressive phenotype. It is contemplated that similar techniques may be used to diagnose and monitor other diseases, for example, fibrosis. Typically, antibodies or antibody-like molecules engineered with a detection moiety (e.g., radiolabel, fluorescence, etc.) can be infused into a patient, which then will distribute and localize to sites of the particular marker (for instance CD8+ and M2 macrophages).

Non-invasive in vivo imaging techniques may be applied in a variety of suitable methods for purposes of diagnosing patients; selecting or identifying patients who are likely to benefit from TGFβ inhibitor therapy such as TGFβ1 inhibitor therapy; and/or, monitoring patients for therapeutic response upon treatment. Any cells with a known cell-surface marker may be detected/localized by virtue of employing an antibody or similar molecules that specifically bind to the cell marker. Typically, cells to be detected by the use of such techniques are immune cells, such as cytotoxic T lymphocytes, regulatory T cells, MDSCs, disease-associated macrophages (M2 macropahges such as TAMs and FAMs), NK cells, dendritic cells, and neutrophils.

Non-limiting examples of suitable immune cell markers include monocyte markers, macrophage markers (e.g., M1 and/or M2 macrophage markers), CTL markers, suppressive immune cell markers, MDSC markers (e.g., markers for G- and/or M-MDSCs), including but are not limited to: CD8, CD3, CD4, CD11 b, CD33, CD163, CD206, CD68, CD14, CD15, CD66b, CD34, CD25, and CD47. In some embodiments, levels of circulating MDSCs (e.g., circulating mMDSCs and/or gMDSCs) may be used to predict, determine, and monitor pharmacological effects of treatment comprising a dose ofTGFβ inhibitor (e.g., a TGFβ1-selective inhibitor, e.g., Ab6). In some embodiments, circulating mMDSCs may be identified by surface markers CD11b+, HLADR−/low, CD14+, CD15−, CD33+/high, and CD66b−. In some embodiments, gMDSCs may be identified by surface markers CD11b+, HLADR−, CD14−, CD15+, CD33+/low, and CD66b+. In some embodiments, LRRC33 is used as a marker for MDSCs. In some embodiments, LRRC33 is used as a marker for MDSCs in circulation. In some embodiments, a reduction in circulating MDSC levels following treatment with a TGFβ inhibitor (e.g., a TGFβ1-selective inhibitor, e.g., Ab6) may be indicative of therapeutic efficacy. In some embodiments, levels of circulating MDSCs may be used to predict, determine, and monitor pharmacological effects of treatment comprising a dose of TGFβ inhibitor (e.g., a TGFβ1-selective inhibitor, e.g., Ab6). Thus, LRRC33 levels in blood samples may serve as a predictive biomarker for assessing therapeutic response in conditions associated with immune suppression.

In vivo imaging techniques described above may be employed to detect, localize and/or track certain MDSCs in a patient diagnosed with a TGFβ1-associated disease, such as cancer and fibrosis. Healthy individuals have no or low frequency of MDSCs in circulation. With the onset of or progression of such a disease, elevated levels of circulating and/or disease-associated MDSCs may be detected. For example, CCR2-positive M-MDSCs have been reported to accumulate to tissues with inflammation and may cause progression of fibrosis in the tissue (such as pulmonary fibrosis), and this is shown to correlate with TGFβ1 expression. Similarly, MDSCs are enriched in a number of solid tumors (including triple-negative breast cancer) and in part contribute to the immunosuppressive phenotype of the TME. Therefore, treatment response to TGFβ inhibition therapy such as a TGFβ1 inhibitor therapy according to the present disclosure may be monitored by localizing or tracking MDSCs. Reduction of or lowfrequency of detectable MDSCs is typically indicative of therapeutic benefits or better prognosis.

Accordingly, the disclosure also includes a method for treating a TGFβ1-related disease or condition which may comprise the following steps: i) selecting a patient diagnosed with a TGFβ1-related disease or condition; and, ii) administering to the patient an antibody or the fragment encompassed herein in an amount effective to treat the disease or condition. In some embodiments, the selection step (i) comprises detection of disease markers (e.g., fibrosis or cancer markers as described herein), wherein optionally the detection comprises a biopsy analysis, serum marker analysis, and/or in vivo imaging. In some embodiments, the selection step (i) comprises an in vivo imaging technique as described herein.

The disclosure also includes a method for treating cancer which may comprise the following steps: i) selecting a patient diagnosed with cancer comprising a solid tumor, wherein the solid tumor is or is suspected to be an immune excluded tumor; and, ii) administering to the patient an antibody or the fragment encompassed herein in an amount effective to treat the cancer. Preferably, the patient has received, is receiving, or is a candidate for receiving a cancer therapy such as an immune checkpoint inhibition therapy (e.g., a PD-(L)1 antibody), a chemotherapy, a radiation therapy, an engineered immune cell therapy, or a cancer vaccine therapy. In some embodiments, the selection step (i) comprises detection of immune cells or one or more markers thereof, wherein optionally the detection comprises a tumor biopsy analysis, serum marker analysis, and/or in vivo imaging. In some embodiments, the selection step (i) comprises an in vivo imaging technique as described here. In some embodiments, the method further comprises monitoring for a therapeutic response as described herein. In certain embodiments, circulating MDSCs, such as G-MDSCs and M-MDSCs, are measured before and after (e.g., 1-7 days or 1-10 weeks before or after) administering a therapeutically effective dose of a TGFβ inhibitor such as a TGFβ1 inhibitor described herein as an indicator of therapeutic efficacy and/or a predictor of response.

In some embodiments, in vivo imaging is performed for monitoring a therapeutic response to the TGFβ1 inhibition therapy in the subject. The in vivo imaging can comprise any one of the imaging techniques described herein and measure any one of the markers and/or parameters described herein. In the case of cancer, the therapeutic response may comprise conversion of an immune excluded tumor into an inflamed tumor (which correlates with increased immune cell infiltration into a tumor), reduced tumor size, and/or reduced disease progression. Increased immune cell infiltration may be visualized by increased intratumoral immune cell frequency or degree of detection signals, such as radiolabeling and fluorescence.

In some embodiments, the in vivo imaging used for diagnosing, selecting, treating, or monitoring patients, comprises MDSC tracking, such as tracking G-MDSCs (also known as PMN-MDSCs) and M-MDSCs. For example, MDSCs (e.g., mMDSCs and/or gMDSCs) may be enriched at a disease site (such as fibrotic tissues and solid tumors) at the baseline. Upon administering a therapy (e.g., a TGFβ1 inhibitor therapy), MDSCs levels may decrease, as measured by reduced intensity of the label (such as radioisotope and fluorescence), indicative of therapeutic effects. In some embodiments, circulating MDSCs, including circulating G-MDSCs and M-MDSCs, may be detected in the blood or a blood component of the subject receiving a TGFβ inhibitor, e.g., a TGFβ1 inhibitor, e.g., Ab6. In some embodiments, MDSCs are detected, measured, or quantified by the use of an antibody that binds LRRC33. In some embodiments, mMDSCs and/or gMDSCs are detected, measured, or quantified by detecting a set of cell surface markers such as those provided herein.

In certain embodiments, assays useful in determining the efficacy and/or therapeutic response in a subject treated with a TGFβ inhibitor (e.g., Ab6) include, but are not limited to, immunohistochemical or immunofluorescence analyses of certain immune cell markers (e.g., flow cytometry or immunohistochemistry) known in the art for measuring levels of circulating MDSCs (e.g., G-MDSCs and M-MDSCs). In some embodiments, human G-MDSCs may be identified by the expression of the surface markers CD11 b+, CD14−, CD33+/low, CD15+, HLADR−, and CD66b+. In some embodiments, human G-MDSCs may also express HLA-DR, LOX-1, and/or Arginase. In some embodiments, M-MDSCs may be identified by the expression of surface markers CD11b+, CD33+/high, CD15−, HLADR−/low, CD66−, and CD14+. In some embodiments, the TGFβ inhibitors such as those encompassed herein may be used to detect reduction in circulatory MDSCs, but not levels of other circulatory monocytes, after administration to a patient in need thereof. In some embodiments, circulatory MDSCs are measured by the expression of cell surface expressed LRRC33.

In some embodiments, the in vivo imaging comprises tracking or localization of LRRC33-positive cells. LRRC33-positive cells include, for example, MDSCs and activated M2-like macrophages (e.g., TAMs and activated macrophages associated with fibrotic tissues). For example, LRRC33-positive cells may be enriched at a disease site (such as solid tumors) at the baseline. Upon therapy (e.g., TGFβ1 inhibitor therapy), fewer cells expressing cell surface LRRC33 may be observed, as measured by reduced intensity of the label (such as radioisotope and fluorescence), indicative of therapeutic effects.

In some embodiments, the in vivo imaging techniques described herein may comprise the use of PET-SPECT, MRI and/or optical fluorescence/bioluminescence in order to detect cells of interest.

In some embodiments, labeling of antibodies or antibody-like molecules with a detection moiety may comprise direct labeling or indirect labeling.

In some embodiments, the detection moiety may be a tracer. In some embodiments, the tracer may be a radioisotope, wherein optionally the radioisotope may be a positron-emitting isotope. In some embodiments, the radioisotope is selected from the group consisting of: 18F, 11C, 13N, 150, 68Ga, 177Lu, 18F and 89Zr.

Thus, such methods may be employed to carry out in vivo imaging with the use of labeled antibodies in immune-PET.

Accordingly, the disclosure also includes a method for treating a TGFβ1 indication in a subject, which method comprises a step of diagnosis, patient selection, and/or monitoring therapeutic effects using an imaging technique. In some embodiments, a TGFβ inhibitor such as an isoform-selective TGFβ1 inhibitor according to the present disclosure is used in the treatment of a TGFβ1 indication, wherein the treatment comprises administration of an effective amount of the TGFβ inhibitor (e.g., Ab6) to treat the indication, and further comprising a step of monitoring therapeutic effects in the subject by in vivo imaging. Optionally, the subject may be selected as a candidate for receiving the TGFβ inhibitor therapy (e.g., a TGFβ1 inhibitor therapy), using a diagnostic or selection step that comprises in vivo imaging. The TGFβ1 indication may be a proliferative disorder (such as cancer with a solid tumor and myelofibrosis).

In some embodiments, the subject has cancer, wherein the method comprises the following steps: i) selecting a patient diagnosed with cancer comprising a solid tumor, wherein the solid tumor is or is suspected to be an immune excluded tumor; and, ii) administering to the patient an antibody or the fragment encompassed herein in an amount effective to treat the cancer. Preferably, the patient has received, or is a candidate for receiving a cancer therapy such as immune checkpoint inhibition therapies (e.g., PD-(L)1 antibodies), chemotherapies, radiation therapies, engineered immune cell therapies, and cancer vaccine therapies. In some embodiments, the selection step (i) comprises detection of immune cells or one or more markers thereof, wherein optionally the detection comprises a tumor biopsy analysis, serum marker analysis, and/or in vivo imaging. In some embodiments, the selection step (i) comprises an in vivo imaging technique as described here. In some embodiments, the method further comprises monitoring for a therapeutic response as described herein.

### Cell-Based Assays for Measuring TGFβ Activation

Activation of TGFβ (and inhibition thereof by a TGFβ test inhibitor, such as an antibody) may be measured by any suitable method known in the art. For example, integrin-mediated activation of TGFβ can be utilized in a cell-based potency assay, such as the “CAGA12” reporter (e.g., luciferase) assay, described in more detail herein. As shown, such an assay system may comprise the following components: i) a source of TGFβ (recombinant, endogenous or transfected); ii) a source of activator such as integrin (recombinant, endogenous, or transfected); and iii) a reporter system that responds to TGFβ activation, such as cells expressing TGFβ receptors capable of responding to TGFβ and translating the signal into a readable output (e.g., luciferase activity in CAGA12 cells or other reporter cell lines). In some embodiments, the reporter cell line comprises a reporter gene (e.g., a luciferase gene) under the control of a TGFβ-responsive promoter (e.g., a PAI-1 promoter). In some embodiments, certain promoter elements that confer sensitivity may be incorporated into the reporter system. In some embodiments, such promoter element is the CAGA12 element. Reporter cell lines that may be used in the assay have been described, for example, in Abe et al., (1994) Anal Biochem. 216(2): 276-84, incorporated herein by reference. In some embodiments, each of the aforementioned assay components are provided from the same source (e.g., the same cell). In some embodiments, two of the aforementioned assay components are provided from the same source, and a third assay component is provided from a different source. In some embodiments, all three assay components are provided from different sources. For example, in some embodiments, the integrin and the latent TGFβ complex (proTGFβ and a presenting molecule) are provided for the assay from the same source (e.g., the same transfected cell line). In some embodiments, the integrin and the TGF are provided for the assay from separate sources (e.g., two different cell lines, a combination of purified integrin and a transfected cell). When cells are used as the source of one or more of the assay components, such components of the assay may be endogenous to the cell, stably expressed in the cell, transiently transfected, or any combination thereof.

A skilled artisan could readily adapt such assays to various suitable configurations. For instance, a variety of sources of TGFβ may be considered. In some embodiments, the source of TGFβ is a cell that expresses and deposits TGFβ (e.g., a primary cell, a propagated cell, an immortalized cell or cell line, etc.). In some embodiments, the source of TGFβ is purified and/or recombinant TGFβ immobilized in the assay system using suitable means. In some embodiments, TGFβ immobilized in the assay system is presented within an extracellular matrix (ECM) composition on the assay plate, with or without de-cellularization, which mimics fibroblast-originated TGFβ. In some embodiments, TGFβ is presented on the cell surface of a cell used in the assay. Additionally, a presenting molecule of choice may be included in the assay system to provide suitable latent-TGFβ complex. One of ordinary skill in the art can readily determine which presenting molecule(s) may be present or expressed in certain cells or cell types. Using such assay systems, relative changes in TGFβ activation in the presence or absence of a test agent (such as an antibody) may be readily measured to evaluate the effects of the test agent on TGFβ activation in vitro. Data from exemplary cell-based assays are provided in the Example section below.

Such cell-based assays may be modified or tailored in a number of ways depending on the TGFβ isoform being studied, the type of latent complex (e.g., presenting molecule), and the like. In some embodiments, a cell known to express integrin capable of activating TGFβ may be used as the source of integrin in the assay. Such cells include SW480/P6 cells (e.g., clone 1E7). In some embodiments, integrin-expressing cells may be co-transfected with a plasmid encoding a presenting molecule of interest (such as GARP, LRRC33, LTBP (e.g., LTBP1 or LTBP3), etc.) and a plasmid encoding a pro-form of the TGFβ isoform of interest (such as proTGFβ1). After transfection, the cells are incubated for sufficient time to allow for the expression of the transfected genes (e.g., about 24 hours), cells are washed, and incubated with serial dilutions of a test agent (e.g., an antibody). Then, a reporter cell line (e.g., CAGA12 cells) is added to the assay system, followed by appropriate incubation time to allow TGFβ signaling. After an incubation period (e.g., about 18-20 hours) following the addition of the test agent, signal/read-out (e.g., luciferase activity) is detected using suitable means (e.g., for luciferase-expressing reporter cell lines, the Bright-Glo reagent (Promega) can be used). In some embodiments, Luciferase fluorescence may be detected using a BioTek (Synergy H1) plate reader, with autogain settings.

Data demonstrating that exemplary antibodies of the disclosure which are capable of selectively inhibiting the activation of TGFβ1 in a context-independent manner can be found, e.g., in PCT/US2019/041390, PCT/US2019/041373, and PCT/US2021/012930.

### Nucleic Acids

In some embodiments, antibodies, antigen binding portions thereof, and/or compositions of the present disclosure may be encoded by nucleic acid molecules. Such nucleic acid molecules include, without limitation, DNA molecules, RNA molecules, polynucleotides, oligonucleotides, mRNA molecules, vectors, plasmids and the like. In some embodiments, the present disclosure may comprise cells programmed or generated to express nucleic acid molecules encoding compounds and/or compositions of the present disclosure. In some cases, nucleic acids of the disclosure include codon-optimized nucleic acids. Methods of generating codon-optimized nucleic acids are known in the art and may include, but are not limited to, those described in U.S. Pat. Nos. 5,786,464 and 6,114,148, the contents of each of which are herein incorporated by reference in their entirety.

### LIST OF CERTAIN EMBODIMENTS

1. A method of treating cancer in a subject, wherein the treatment comprises administering to the subject a TGFβ inhibitor in an amount sufficient to reduce circulating MDSC levels.

2. The method of embodiment 1, wherein the reduced circulating MDSCs are G-MDSCs.

3. The method of embodiment 1 or 2, wherein the G-MDSCs express one or more of LOX-1+, CD11 b+, HLADR−, CD14−, CD15+, CD33+/low, and CD66+.

4. The method of any one of embodiments 1-3, wherein the treatment further comprises administering a cancer therapy.

5. The method of any one of embodiments 1-4, wherein the TGFβ inhibitor and the cancer therapy are administered concurrently (e.g., simultaneously), separately, or sequentially.

6. The method of embodiment 4 or 5, comprising determining whether a subject has a reduction in circulating MDSC levels following administration of the TGFβ inhibitor, and administering the cancer therapy if MDSC levels have been reduced.

7. The method of any one of embodiments 1-6, wherein the cancer therapy comprises a checkpoint inhibitor therapy, optionally an agent targeting PD-1 or PD-L1, optionally an anti-PD-1 or anti-PD-L1 antibody.

8. A method of predicting therapeutic efficacy in a subject having cancer, comprising:
(i) determining circulating MDSC levels in the subject prior to administering a TGFβ inhibitor (alone or in combination with a cancer therapy);
(ii) administering to the subject a therapeutically effective amount of the TGFβ inhibitor (alone or in combination with a cancer therapy); and
iii) determining circulating MDSC levels in the subject after the administration, wherein a reduction in circulating MDSC levels after administration, as compared to circulating MDSC levels before administration, predicts pharmacological effects.

9. A method of treating cancer in a subject, comprising the steps of:
(i) determining circulating MDSC levels in the subject prior to administering a TGFβ inhibitor;
(ii) administering to the subject a first therapeutically effective dose of the TGFβ inhibitor;
(iii) determining circulating MDSC levels in the subject after administering the TGFβ inhibitor;
(iv) administering to the subject a second therapeutically effective dose of the TGFβ inhibitor if the circulating MDSC levels measured after administering the first therapeutically effective dose of the TGFβ inhibitor are reduced as compared to the circulating MDSC levels measured prior to administering the first therapeutically effective dose of the TGFβ1 inhibitor.

10. The method of embodiment 8 or embodiment 9, comprising administering a checkpoint inhibitor therapy concurrently (e.g., simultaneously), separately, or sequentially with the TGFβ inhibitor.

11. A method of treating cancer in a subject, comprising the steps of:
(i) determining circulating MDSC levels in the subject prior to administering a combination therapy comprising a therapeutically effective amount of a TGFβ inhibitor and a therapeutically effective amount of a checkpoint inhibitor therapy;
(ii) administering to the subject the combination therapy;
(iii) determining circulating MDSC levels in the subject after administering the combination therapy;
(iv) continuing the combination therapy if the circulating MDSC levels measured after administering the first therapeutically effective dose of the combination therapy are reduced as compared to the circulating MDSC levels measured prior to administering the first therapeutically effective dose.

12. The method of embodiment 11, wherein the combination therapy comprises administering the TGFβ inhibitor concurrently (e.g., simultaneously), separately, or sequentially with the checkpoint inhibitor therapy.

13. A method of treating advanced cancer in a human subject, the method comprising the steps of
i) selecting a subject with advanced cancer comprising a locally advanced tumor and/or metastatic cancer with primary resistance to a checkpoint inhibitor therapy,
ii) administering a TGFβ inhibitor; and,
ii) administering to the subject a checkpoint inhibitor therapy.

14 The method of embodiment 13, wherein the checkpoint inhibitor therapy is administered concurrently (e.g., simultaneously), separately, or sequentially with the TGFβ inhibitor.

15. A method for treating advanced cancer in a human subject, the method comprising the steps of
i) selecting a subject with advanced cancer comprising a locally advanced tumor and/or metastatic cancer with primary resistance to a CPI therapy, wherein the subject has received a TGFβ inhibitor which is a TGFβ1-selective inhibitor or a TGFβ inhibitor that does not inhibit TGFβ3; and,
ii) administering to the subject a CPI therapy, optionally in conjunction with the TGFβ inhibitor.

16 The method of any one of embodiments 13-15, further comprising measuring the levels of circulating MDSC levels before and after administering the treatment, wherein a reduction in circulating MDSC levels is indicative of a treatment response.

17. The method of embodiment 16, further comprising continuing the treatment if a reduction in circulating MDSC levels is determined.

18. The method of any one of embodiments 8-17, wherein the reduced circulating MDSCs are G-MDSCs.

19. The method of embodiment 18, wherein the G-MDSCs express one or more of LOX-1+, CD11 b+, HLADR−, CD14−, CD15+, CD33+/low, and CD66+.

20. The method of any one of embodiments 1-19, wherein the circulating MDSC levels are determined from whole blood or a blood component collected from the subject.

21 The method of any one of embodiments 1-20, wherein the treatment reduces circulating MDSC levels by at least 10%, optionally by at least 15%, 20%, 25%, or more.

22. The method of any one of embodiments 1-21, wherein the TGFβ inhibitor is a TGFβ1 inhibitor, optionally a TGFβ1-specific inhibitor.

23 The method of any one of embodiments 1-21, wherein the subject has circulating MDSC levels at least 2-fold above circulating MDSC levels in a healthy subject prior to a treatment.

24 A method of selecting a subject for treatment, wherein the subject has circulating MDSC levels at least 2-fold above circulating MDSC levels in a healthy subject prior to treatment.

25 The method of 24, wherein the subject has or is suspected of having cancer.

26 A method of treating a subject for cancer, wherein the subject has circulating MDSC levels at least 2-fold above circulating MDSC levels in a healthy subject prior to treatment, comprising administering to the subject a TGFβ inhibitor in an amount sufficient to reduce circulating MDSC levels.

27 The method of any one of embodiments 1-26, wherein the level of circulating MDSC cells is determined within 3-6 weeks, e.g., within or at about 3 weeks, following administration of a TGFβ inhibitor.

28 The method of any one of embodiments 1-27, wherein the level of circulating MDSC cells is determined within 2 weeks, e.g., 10 days, following administration of the TGFβ inhibitor.

29. The method of any one of embodiments 1-28, further comprising the steps of:
(i) determining the levels of tumor-associated immune cells in the subject prior to administering a treatment;
(ii) administering the treatment to the subject; and
(iii) determining the levels of tumor-associated immune cells in the subject after administering the treatment;
   wherein a change in the level of one or more tumor-associated immune cell populations after inhibitor administration, as compared to the levels of tumor-associated immune cells before administration, indicates therapeutic efficacy.

30. The method of embodiment 29, wherein a change in levels of tumor-associated immune cells in step (iii) indicates reduction or reversal of immune suppression in the cancer.

31. The method of embodiment 29 or 30, wherein the tumor-associated immune cells comprise CD8+ T cells and/or tumor-associated macrophages (TAMs).

32. The method of embodiments 29-31, wherein the change in the levels of tumor-associated immune cells comprises at least a 10%, optionally at least a 15%, 20%, 25%, or more, increase in CD8+ T cell levels.

33. The method of embodiments 29-32, wherein the change in the levels of tumor-associated immune cells comprises at least a 10%, optionally at least a 15%, 20%, 25%, or more, increase in the level of TAMs.

34. The method of any one of embodiments 29-33, wherein the levels of tumor-associated immune cells are determined in a sample collected from the subject by immunohistochemistry analysis.

35. The method of any one of embodiments 29-34, wherein the levels of tumor-associated immune cells are determined by in vivo imaging.

36. The method of any one of embodiments 29-34, wherein the sample is a tumor biopsy sample. 37. The method of any one of embodiments 29-36, further comprising continuing to administer the treatment

if a change in the level of one or more tumor-associated immune cell populations is detected.

38. The method of any one of embodiments 1-37, further comprising the steps of:
(i) determining the levels of circulating latent TGFβ in the subject prior to administering a treatment;
(ii) administering the treatment to the subject; and
(iii) determining the levels of circulating latent TGFβ in the subject after administering the treatment; and
wherein an increase in circulating latent TGFβ after inhibitor administration, as compared to circulating latent TGFβ before administration, indicates therapeutic efficacy.

39. The method of embodiment 38, further comprising continuing to administer the treatment if a change in the level of circulating latent TGFβ is detected.

40 The method of 38 or 39, wherein the level of circulating latent TGFβ is determined in a sample obtained from the subject.

41 The method of 40, wherein the sample is a whole blood sample or a blood component.

42 The method of any one of embodiments 39-41, wherein the circulating latent TGFβ is circulating latent TGFβ1.

43. A method of treating cancer, comprising administering to a subject a TGFβ inhibitor in a therapeutically effective amount that does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1β), and chemokine C—C motif ligand 2 (CCL2)/monocyte chemoattractant protein 1 (MCP-1).

44. A method for identifying whether a TGFβ inhibitor will be tolerated in a patient, comprising contacting a cell culture or fluid sample with the TGFβ inhibitor and determining whether it causes a significant release of one or more cytokines selected from interferon gamma (IFNγ), interleukin 2 (IL-2), interleukin 6 (IL-6), tumor necrosis factor alpha (TNFα), interleukin 1 beta (IL-1p) and chemokine C—C motif ligand 2 (CCL2)/monocyte chemoattractant protein 1 (MCP-1), wherein a significant release indicates the TGFβ inhibitor will not be well tolerated.

45. The method of embodiment 43 or 44, wherein cytokine release is assessed in an in vitro cytokine release assay, optionally an assay in peripheral blood mononuclear cells (PBMCs) or whole blood, optionally wherein the PBMCs or whole blood are obtained from the subject prior to administering a TGFβ inhibitor therapy.

46. The method of any one of embodiments 43-45, wherein cytokine release is assessed in an in vitro cytokine release assay of peripheral blood mononuclear cells (PBMCs) or whole blood obtained from a healthy subject.

47. The method of embodiment 45 or 46, wherein the cytokine release assay comprises a soluble phase and/or a solid phase assay format.

48. The method of any one of embodiments 45-47, wherein the cytokine release assay comprises: i) a solid phase assay, ii) a high-density PBMC pre-culture assay, and/or iii) a PBL-HUVEC co-culture assay.

49. The method of any one of embodiments 45-48, wherein the cytokine release assay comprises a multiplex array, e.g., a Luminex® array system.

50. The method of any one of embodiments 45-49, wherein the cytokine release assay comprises comparing cytokine release from a TGFβ inhibitor to release from one or more control antibodies selected from an anti-CD3 antibody and an anti-CD28 antibody, optionally wherein the CD28 antibody optionally comprises TGN1412.

51. The method of any one of embodiments 43-50, wherein the TGFβ inhibitor does not induce more than a 10-fold increase in IL-6 levels, optionally less than a 2-fold, 4-fold, 6-fold, or 8-fold increase in IL-6 levels, as compared to levels in the absence of the inhibitor or in the presence of a control antibody.

52. The method of any one of embodiments 43-51, wherein the TGFβ inhibitor does not induce more than a 10-fold increase in IFNγ levels, optionally less than a 2-fold, 4-fold, 6-fold, or 8-fold increase in IFNγ levels, as compared to levels in the absence of the inhibitor or in the presence of a control antibody.

53. The method of embodiments 43-52, wherein the TGFβ inhibitor does not induce more than a 10-fold increase in TNFα levels, optionally less than a 2-fold, 4-fold, 6-fold, or 8-fold increase in TNFα levels, as compared to levels in the absence of the inhibitor or in the presence of a control antibody.

54. The method of embodiment any one of embodiments 43-53, wherein the TGFβ inhibitor is administered in a therapeutically effective amount that does not cause a significant release of one or more cytokines in an animal model comprising a non-human primate.

55. The method of embodiments 43-54, wherein the therapeutically effective amount of the TGFβ inhibitor is an amount sufficient to reduce circulating MDSC levels.

56. The method of embodiment 55, wherein the reduced MDSCs are G-MDSCs.

57. The method of embodiment 56, wherein the G-MDSCs express one or more of LOX-1+, CD11 b+, HLADR−, CD14−, CD15+, CD33+/low, and CD66+.

58. The method of any one of embodiments 55-57, wherein the circulating MDSC levels are determined from whole blood or a blood component collected from the subject.

59. The method of any one of embodiments 43-58, wherein the TGFβ inhibitor is a TGFβ1 inhibitor, optionally a TGFβ1-specific inhibitor, e.g., Ab6.

60. A TGFβ inhibitor for use in the treatment of cancer in a subject, wherein the treatment comprises administration of a dose of said TGFβ inhibitor to the subject having cancer, wherein said TGFβ inhibitor does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1p) and chemokine C—C motif ligand 2 (CCL2)/monocyte chemoattractant protein 1 (MCP-1).

61. A combination therapy comprising a dose of a TGFβ inhibitor and a cancer therapy agent for use in the treatment of cancer, wherein the treatment comprises simultaneous, separate or sequential administration to a subject of a dose of the TGFβ inhibitor and the cancer therapy agent, wherein said TGFβ inhibitor does not cause a significant release of one or more cytokines selected from interferon gamma (IFNγ), interleukin 2 (IL-2), interleukin 6 (IL-6), tumor necrosis factor alpha (TNFα), interleukin 1 beta (IL-1p) and chemokine C—C motif ligand 2 (CCL2)/monocyte chemoattractant protein 1 (MCP-1).

62. The TGFβ inhibitor for use according to embodiment 60 or the combination therapy for use according to embodiment 61, wherein the TGFβ inhibitor is administered in a therapeutically effective amount that does not cause a significant release of one or more cytokines in an animal model comprising a non-human primate.

63. The TGFβ inhibitor for use according to embodiment 60 or 62, or the combination therapy for use according to embodiment 61, wherein the TGFβ inhibitor is administered in a therapeutically effective amount that is sufficient to reduce circulating MDSC levels.

64 The TGFβ inhibitor for use or the combination therapy for use according to embodiment 63, wherein the reduced MDSCs are G-MDSCs.

65 The TGFβ inhibitor for use or the combination therapy for use according to embodiment 64, wherein the G-MDSCs express one or more of LOX-1+, CD11b+, HLADR−, CD14−, CD15+, CD33+/low, and CD66+.

66 The TGFβ inhibitor for use or the combination therapy for use according to any one of embodiments 63-65, wherein the circulating MDSC levels are determined from whole blood or a blood component collected from the subject.

67. The method of embodiment 44 or any embodiment dependent thereon, the TGFβ inhibitor for use according to embodiment 60 or any embodiment dependent thereon, or the combination therapy for use according to embodiment 61 or any embodiment dependent thereon, wherein the TGFβ inhibitor is a TGFβ1 inhibitor, optionally a TGFβ1-specific inhibitor.

68. The TGFβ inhibitor for use according to embodiment 60 or any embodiment dependent thereon, or the combination therapy for use according to embodiment 61 or any embodiment dependent thereon, wherein the TGFβ inhibitor does not cause significant release of one or more cytokines as determined by the method of embodiment 44 or any embodiment dependent thereon.

69. A method of treating cancer, comprising administering to a subject a TGFβ inhibitor in a therapeutically effective amount that does not induce a significant increase in platelet binding, activation, and/or aggregation.

70. The method of embodiment 69, wherein platelet binding, activation, and/or aggregation is measured in a sample of plasma or whole blood.

71. A method for determining whether a TGFβ inhibitor causes a significant increase in platelet binding, activation and/or aggregation following exposure of the sample to said TGFβ inhibitor, which method comprises measuring platelet binding, activation and/or aggregation in a blood sample.

72 The method of any one of embodiments 70 or 71, wherein the sample is obtained from the subject prior to administering a TGFβ inhibitor therapy.

73. The method of any one of embodiment 69-72, wherein the sample is obtained from a healthy subject.

74. The method of any one of embodiments 69-73, wherein administering the TGFβ inhibitor does not increase platelet binding by more than 10% as compared to binding in the absence of the TGFβ inhibitor and/or in the presence of a buffer or isotype control.

75. The method of any one of embodiments 69-74, wherein administering the TGFβ inhibitor does not increase platelet activation by more than 10%, as compared to activation in the absence of the inhibitor.

76. The method of any one of embodiments 69-75, wherein administering the TGFβ inhibitor does not increase platelet aggregation in vitro by more than 10% of the activation induced by a known platelet aggregation agonist, e.g., adenosine diphosphate (ADP).

77. The method of any one of embodiments 69-76, wherein administering the TGFβ inhibitor does not increase platelet aggregation by more than 10%, as compared to aggregation caused by a negative control.

78. The method of embodiments any one of 69-77, wherein the therapeutically effective amount of the TGFβ inhibitor is an amount sufficient to reduce levels of circulating MDSCs.

79. The method of embodiment 78, wherein the reduced MDSCs are G-MDSCs.

80. The method of embodiment 79, wherein the G-MDSCs express one or more of LOX-1+, CD11 b+, HLADR−, CD14−, CD15+, CD33+/low, and CD66+.

81. The method of any one of embodiments 78-80, wherein the circulating MDSC levels are determined from whole blood or a blood component collected from the subject.

82. The method of any one of embodiments 69-81, wherein the TGFβ inhibitor is a TGFβ1 inhibitor, optionally a TGFβ1-specific inhibitor.

83. A TGFβ inhibitor for use in the treatment of cancer by administering to a subject a dose of said TGFβ inhibitor, wherein said TGFβ inhibitor does not cause a significant increase in platelet binding, activation and/or aggregation.

84. A combination therapy comprising a dose of a TGFβ inhibitor and a cancer therapy agent for the treatment of cancer, wherein the treatment comprises simultaneous, separate, or sequential administration to a subject of a dose of the TGFβ inhibitor and the cancer therapy agent, wherein said TGFβ inhibitor does not cause a significant increase in platelet binding, activation and/or aggregation.

85. The TGFβ inhibitor for use according to embodiment 83 or the combination therapy for use according to embodiment 84, wherein the TGFβ inhibitor is administered in a therapeutically effective amount that is sufficient to reduce circulating MDSC levels.

86 The TGFβ inhibitor for use or the combination therapy for use according to embodiment 85, wherein the reduced MDSCs are G-MDSCs.

87 The TGFβ inhibitor for use or the combination therapy for use according to embodiment 85, wherein the M-MDSCs express one or more of LOX-1+, CD11b+, HLADR−, CD14−, CD15+, CD33+/low, and CD66+.

88 The TGFβ inhibitor for use or the combination therapy for use according to embodiment 85, wherein the reduced MDSCs are M-MDSCs that express one or more of CD11b+, HLADR−/low, CD14+, CD15−, CD33+/high, and CD66b−.

89. The method of embodiment 71 or any embodiment dependent thereon, the TGFβ inhibitor for use according to embodiment 83 or any embodiment dependent thereon, or the combination therapy for use according to embodiment 84 or any embodiment dependent thereon, wherein the TGFβ inhibitor is a TGFβ1 inhibitor, optionally a TGFβ1-specific inhibitor.

90. The TGFβ inhibitor for use according to embodiment 83 or any embodiment dependent thereon, or the combination therapy for use according to embodiment 84 or any embodiment dependent thereon, wherein the TGFβ inhibitor has been determined not to cause a significant increase in platelet binding, activation and/or aggregation by the method of embodiment 71 or any embodiment dependent thereon.

91. The TGFβ inhibitor for use according to embodiment 83 or any embodiment dependent thereon, or the combination therapy for use according to embodiment 84 or any embodiment dependent thereon, wherein the TGFβ inhibitor is a TGFβ inhibitor according to any one of embodiments 60-68.

92. A method of making a TGFβ inhibitor for treating cancer in a subject, comprising the steps of selecting a TGFβ inhibitor which satisfies one or more, e.g., all of, the following criteria:
a) the TGFβ inhibitor is efficacious in one or more preclinical models;
b) the TGFβ inhibitor does not cause valvulopathies or epithelial hyperplasia in toxicology studies in one or more animal species at a dose at least greater than a minimum efficacious dose; and
c) the TGFβ inhibitor does not induce significant cytokine release from human PBMCs or whole blood in an in vitro cytokine release assay at the minimum efficacious dose as determined in the one or more preclinical models of (a);

93 A method of making a TGFβ inhibitor for treating cancer in a subject, comprising the steps of selecting a TGFβ inhibitor which satisfies one or more, e.g., all of, the following criteria:
a) the TGFβ inhibitor is efficacious in one or more preclinical models;
b) the TGFβ inhibitor does not cause valvulopathies or epithelial hyperplasia in toxicology studies in one or more animal species at a dose at least greater than a minimum efficacious dose;
c) the TGFβ inhibitor does not induce significant cytokine release from human PBMCs or whole blood in an in vitro cytokine release assay at the minimum efficacious dose as determined in the one or more preclinical models of (a);
d) the TGFβ inhibitor does not induce a significant increase in platelet binding, activation, and/or aggregation at the minimum efficacious dose as determined in the one or more preclinical models of (a); and
e) the TGFβ inhibitor reduces circulating MDSCs at the minimum efficacious dose as determined in the one or more preclinical models of (a),
   wherein the method further comprises manufacturing a pharmaceutical composition comprising the TGFβ inhibitor and a pharmaceutically acceptable excipient.

94. The method of embodiment 92 or 93, wherein the TGFβ inhibitor is a TGFβ1 inhibitor, optionally a TGFβ1-specific inhibitor.

95. A method of treating cancer in a subject, comprising administering a therapeutically effective amount of the TGFβ inhibitor manufactured according to the method of any one of embodiments 92-94.

96. A TGFβ inhibitor for use in an intermittent dosing regimen for cancer immunotherapy in a patient, wherein the intermittent dosing regimen comprises:
(i) measuring circulating MDSCs in a first sample, e.g., a blood sample, collected from the patient prior to a TGFβ inhibitor treatment,
(ii) administering a TGFβ inhibitor to the patient treated with a cancer therapy, wherein the cancer therapy is optionally a checkpoint inhibitor therapy,
(iii) measuring circulating MDSCs in a second sample collected from the patient after the TGFβ inhibitor treatment,
(iv) continuing with the cancer therapy if the second sample shows reduced levels of circulating MDSCs as compared to the first sample; and
(v) repeating the process as needed after a further blood sample from a patient shows elevated levels of circulating MDSC levels.

97 The method of embodiment 96, further comprising measuring circulating MDSCs in a third sample, and administering to the patient an additional dose of a TGFβ inhibitor if the third sample shows elevated levels of circulating MDSC levels as compared to the second sample.

98. The method of embodiment 96 or 97, wherein the TGFβ inhibitor inhibits TGFβ1 signaling.

99. The method of embodiment 96 or 97, wherein the TGFβ inhibitor inhibits TGFβ1 signaling but does not inhibit TGFβ2 signaling and/or TGFβ3 signaling at a therapeutically effective dose.

100. The method of embodiment 96 or 97, wherein the TGFβ inhibitor is TGFβ31-selective.

101. The method of embodiment 96 or 97, wherein the TGFβ inhibitor is an integrin inhibitor.

102. The method of 101, wherein the integrin inhibitor inhibits integrin αVβ1, αVβ3, αVβ5, αVβ6, αVβ8, α5β1, αIIbβ3, and/or α8β1.

103. The method of 101 or 102, wherein the integrin inhibitor inhibits downstream TGFβ1/3 activation.

104. A TGFβ1-selective inhibitor for use in the treatment of cancer in a subject, wherein the subject has been treated with a TGFβ inhibitor that inhibits TGFβ3 in conjunction with a checkpoint inhibitor.

105. The method of 104, wherein the cancer is a metastatic cancer, a desmoplastic tumor, or myelofibrosis.

106. The method of 104 or 105, wherein the subject has a disorder involving dysregulated extracellular matrix (ECM) or is at risk of developing such a disorder.

107. The method of 106, wherein the disorder involving dysregulated ECM is NASH,

108. The TGFβ1-selective inhibitor for use according to any one of embodiments 104-107, wherein the prior TGFβ inhibitor inhibits TGFβ1/2/3 or TGFβ1/3.

109. A non-isoform-selective TGFβ inhibitor for use in the treatment of cancer in a subject, comprising the steps of:
(i) selecting a subject who is not diagnosed with a fibrotic disorder or who is not at high risk of developing a fibrotic disorder; and,
(ii) administering to the subject the non-isoform-selective TGFβ inhibitor in an amount effective to treat the cancer.

110. An isoform-non-selective TGFβ inhibitor for use in the treatment of cancer in a subject, wherein the treatment comprises the steps of selecting a subject whose cancer is not a highly metastatic cancer and administering to the subject the isoform-non-selective TGFβ inhibitor.

111. The method of 109 or 110, wherein the isoform-non-selective TGFβ inhibitor is an antibody that inhibits TGFβ1/2/3 or TGFβ1/3.

112 The method of any one of embodiments 109-111, wherein the isoform-non-selective TGFβ inhibitor is an integrin inhibitor binding to integrins αVβ1, αVβ6, αVβ8, and/or αVβ3.

113 The method of 112, wherein the integrin inhibitor is an inhibitor of TGFβ1/3 activation.

114. The method of 110, wherein the isoform-non-selective TGFβ inhibitor is an engineered construct comprising a TGFβ receptor ligand-binding moiety.

115. The isoform-non-selective TGFβ inhibitor for use according to embodiment 110, wherein the highly metastatic cancer is colorectal cancer, lung cancer (e.g., NSCLC), bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, or thyroid cancer.

116. A TGFβ1-selective inhibitor for use in the treatment of cancer in a subject wherein the treatment comprises the steps of
(i) selecting a subject whose cancer is a highly metastatic cancer, and
(ii) administering to the subject an isoform-selective TGFβ1 inhibitor;
   wherein the highly metastatic cancer comprises colorectal cancer, lung cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, or thyroid cancer.

117. A TGFβ1-selective inhibitor for use in the treatment of cancer in a subject wherein the treatment comprises the steps of:
(i) selecting a subject having myelofibrosis, a fibrotic disorder or is at risk of developing a fibrotic disorder, and,
(ii) administering to the subject an isoform-selective TGFβ1 inhibitor in an amount effective to treat the cancer.

118. The TGFβ1-selective inhibitor for use according to embodiment 117, wherein the subject is further treated with a cancer therapy, wherein optionally the cancer therapy comprises a checkpoint inhibitor.

119. The method, the medical use, the TGFβ inhibitor for use, or the combination therapy for use according to any one of the preceding embodiments, wherein the subject is a patient who has not received cancer therapy.

120. The method, the medical use, the TGFβ inhibitor for use, or the combination therapy for use according to any one of the preceding embodiments, wherein the subject is receiving cancer therapy.

121. The method, the medical use, the TGFβ inhibitor for use, or the combination therapy for use according to any one of the preceding embodiments, wherein the subject has previously received cancer therapy.

122. The method, the medical use, the TGFβ inhibitor for use, or the combination therapy for use according to any one of the preceding embodiments, wherein the subject is or will be receiving cancer therapy.

123. The method, the medical use, the TGFβ inhibitor for use, or the combination therapy for use according to any one of the preceding embodiments, wherein the subject has cancer that is resistant to a cancer therapy that does not comprise a TGFβ inhibitor.

124. The method, the medical use, the TGFβ inhibitor for use, or the combination therapy for use according to any one of the preceding embodiments, wherein the subject is poorly responsive to a cancer therapy that does not comprise a TGFβ inhibitor.

125. The method, the medical use, the TGFβ inhibitor for use, or the combination therapy for use according to any one of the preceding embodiments, wherein the subject is currently receiving or previously received a cancer therapy that does not comprise a TGFβ inhibitor.

126. The method, the medical use, the TGFβ inhibitor for use, or the combination therapy for use according to any one of embodiments 121-125, wherein the cancer therapy does not comprise a TGFβ inhibitor.

127. The method, the medical use, the TGFβ inhibitor for use, or the combination therapy for use according to any one of embodiments 121-126, wherein the cancer therapy comprises a chemotherapy, radiation therapy (e.g., a radiotherapeutic agent), engineered immune cell therapy (e.g., CAR-T therapy), oncolytic viral therapy, and/or cancer vaccine therapy.

128. The method, the medical use, the TGFβ inhibitor for use, or the combination therapy for use according to any one of embodiments 121-127, wherein the cancer therapy comprises immunotherapy comprising a checkpoint inhibitor therapy.

129. A method of treating a subject having a solid cancer, comprising determining the level of cytotoxic T cells (e.g., CD8+ T cells) in a sample obtained from the subject prior to administering a TGFβ inhibitor, wherein the level of cytotoxic T cells (e.g., CD8+ T cells) inside the tumor is lower than the level of cytotoxic T cells (e.g., CD8+ T cells) outside the tumor prior to treatment, and administering to the subject a therapeutically effective amount of a TGFβ inhibitor, wherein the therapeutically effective amount is an amount sufficient to increase the level of cytotoxic T cells (e.g., CD8+ T cells) inside the tumor relative to the level outside the tumor.

130. A method of treating a subject having a solid cancer, comprising determining in a sample obtained from the subject the cytotoxic T cell (e.g., CD8+ T cell) levels inside and outside the tumor, selecting a subject having a ratio of cytotoxic T cell (e.g., CD8+ T cell) levels inside the tumor to outside the tumor of less than 1, and administering to the subject a therapeutically effective amount of a TGFβ inhibitor.

131. The method of embodiment 129 or 130, wherein the level of cytotoxic T cells (e.g., CD8+ T cells) outside the tumor is determined from the tumor margin and/or stroma.

132. The method of any one of embodiments 129-131, wherein the level of cytotoxic T cells (e.g., CD8+ T cells) outside of the tumor is determined from the margin.

133. The method of embodiment 131 or embodiment 132, wherein the margin is approximately 10-100 μm in width (e.g., 50 μm in width).

134. The method of any one of embodiments 129-133, wherein the level of the cytotoxic T cells (e.g., CD8+ T cells) in the margin and/or the stroma is at least 2-fold, 3-fold, 4-fold, 5-fold, 7-fold, or 10-fold greater than the level inside the tumor.

135. A method of treating a subject having a solid cancer, comprising measuring levels of CD8+ cells in one or more tumor nests from at least one tumor tissue sample obtained from the subject, and administering to the subject a therapeutically effective amount of a TGFβ inhibitor if greater than 50% of the area of the sample measured comprises tumor nests comprising lower levels of CD8-positive cells inside the tumor nest relative to levels of CD8-positive cells outside of the tumor nest (e.g., less than 5% CD8+ cells inside the tumor nest and greater than 5% CD8+ cells outside the tumor nest).

136. A method of treating a subject having a solid cancer, comprising:
(i) determining the cytotoxic T cell (e.g., CD8+ T cell) levels inside and outside the tumor in a first sample and selecting a subject having a ratio of cytotoxic T cell (e.g., CD8+ T cell) density inside the tumor to density outside the tumor of less than 1;
(ii) administering to the subject a first dose of a TGFβ inhibitor; and
(iii) determining the level of cytotoxic T cells (e.g., CD8+ T cells) inside the tumor in a second sample; and
(iv) administering to the subject a second dose of the TGFβ inhibitor if the level of cytotoxic T cells (e.g., CD8+ T cells) inside the tumor determined in step (iii) is increased as compared to the level of cytotoxic T cells (e.g., CD8+ T cells) inside the tumor determined in step (i).

137. A method of determining therapeutic efficacy of a cancer treatment in a subject comprising:
(i) determining the level of cytotoxic T cells (e.g., CD8+ T cells) inside the tumor in a first sample;
(ii) administering to the subject a dose of a TGFβ inhibitor;
(iii) determining the level of cytotoxic T cells (e.g., CD8+ T cells) inside the tumor in a second sample; and
(iv) determining whether the level of cytotoxic T cells (e.g., CD8+ T cells) determined in step (iii) is increased as compared to step (i), such increase being indicative of therapeutic efficacy of the cancer treatment.

138. The method of embodiment 136 or embodiment 137, wherein step (ii) further comprises administering to the subject an additional cancer therapy simultaneously, separately, or sequentially to the TGFβ inhibitor.

139. The method of embodiment 138, wherein the cancer therapy comprises a checkpoint inhibitor therapy (e.g., an agent targeting PD-1 or PD-L1, or an anti-PD-1 or anti-PD-L1 antibody).

140. The method of any one of embodiments 136-139, wherein the level of cytotoxic T cells (e.g., CD8+ T cells) in the tumor is increased by at least 10%, 15%, 20%, 25%, or more.

141. The method of any one of embodiments 129-140, wherein the TGFβ inhibitor is a TGFβ1-selective inhibitor, e.g., Ab6.

142. The method of any one of embodiments 129-141, wherein the sample is a tumor biopsy sample.

143. The method of embodiment 142, wherein the tumor biopsy sample is a core needle biopsy sample of the tumor.

144. The method of any of embodiments 129-143, wherein the level of cytotoxic T cells (e.g., CD8+ T cells) are determined by immunohistochemical analysis.

145. The method of embodiment 136, further comprising determining the level of circulating MDSCs before and after administration of the first dose of the TGFβ inhibitor, wherein a second dose of the TGFβ inhibitor is administered if a reduction of MDSC levels is determined after the administration of the first dose of the TGFβ inhibitor and the level of cytotoxic T cells (e.g., CD8+ T cells) inside the tumor determined in step (iii) is increased as compared to the level of cytotoxic T cells (e.g., CD8+ T cells) inside the tumor determined in step (i).

146. The method of embodiment 137, further comprising determining the level of circulating MDSCs before and after administration of the TGFβ inhibitor, wherein a reduction of MDSC levels and/or an increase in cytotoxic T cells (e.g., CD8+ T cells) levels inside the tumor after the administration indicates therapeutic efficacy.

147 The method of embodiment 145 or 146, wherein the circulating MDSCs are G-MDSCs.

148. The method of embodiment 147, wherein the G-MDSCs express one or more of LOX-1+, CD11b+, HLADR−, CD14−, CD15+, CD33+/low, and CD66+.

149. The method of any one of embodiments 145-148, wherein the circulating MDSC levels are determined from whole blood or a blood component collected from the subject.

150. The method of any one of embodiments 145-149, wherein the level of circulating MDSC levels is reduced by at least 10%, optionally by at least 15%, 20%, 25%, or more.

151. The method of any one of embodiments 145-150, wherein the level of cytotoxic T cells (e.g., CD8+ T cells) inside the tumor is increased by at least 10%, 15%, 20%, 25%, or more, and the level of circulating MDSCs is decreased by at least 15%, 20%, 25%, or more.

152. The method of any one of embodiments 129-151, wherein the level of cytotoxic T cells (e.g., CD8+ T cells) is the percentage of CD8+ T cells or the CD8+ cell density (e.g., number of CD8+ T cells per millimeter squared).

153. The method of any one of embodiments 129-152, wherein the therapeutically effective amount of the TGFβ inhibitor is between 0.1 mg/kg to 30 mg/kg per dose.

154. The method of any one of embodiments 129-153, wherein the therapeutically effective amount of the TGFβ inhibitor is between 1 mg/kg and 10 mg/kg per dose.

155. The method of any one of embodiments 129-154, wherein the therapeutically effective amount of the TGFβ inhibitor is between 2 mg/kg and 7 mg/kg per dose.

156. The method of any one of embodiments 129-155, wherein the TGFβ inhibitor is dosed weekly, every 2 weeks, every 3 weeks, every 4 weeks, monthly, every 6 weeks, every 8 weeks, bimonthly, every 10 weeks, every 12 weeks, every 3 months, every 4 months, every 6 months, every 8 months, every 10 months, or once a year.

157. The method of any one of embodiments 129-156, wherein the TGFβ inhibitor is dosed about every 3 weeks.

158. The method of any one of embodiments 129-157, wherein the TGFβ inhibitor is administered intravenously or subcutaneously.

159. The method of any one of embodiments 129-158, wherein the cancer is non-small cell lung cancer, melanoma, renal cell carcinoma, triple-negative breast cancer, gastric cancer, microsatellite stable-colorectal cancer, pancreatic cancer, small cell lung cancer, HER2-positive breast cancer, or prostate cancer.

160. A method of determining therapeutic efficacy of a cancer treatment in a subject, wherein the treatment comprises administering to the subject a combination therapy for simultaneous, separate or sequential administration comprising a dose of a TGFβ inhibitor and a cancer therapy, which method comprises:
(i) determining the circulating myeloid-derived suppressor cell (MDSC) level in a sample obtained from the subject prior to administering the TGFβ inhibitor, wherein optionally the MDSC level is determined by the use of an antibody that binds LRRC33;
(ii) determining the circulating MDSC level in a sample obtained from the subject after the administration of the TGFβ inhibitor, wherein optionally the MDSC level is determined by the use of an antibody that binds LRRC33; and
(iii) determining whether the level determined in step (ii) is reduced compared to the level determined in step (i), such reduction being indicative of therapeutic efficacy of the cancer treatment.

161. The method of embodiment 160, wherein the level of circulating MDSC cells is determined within 3-6 weeks following administration of the dose of TGFβ inhibitor, optionally within 3 weeks or at about 3 weeks following administration of the dose of TGFβ inhibitor.

162. The method of embodiment 160, wherein the level of circulating MDSC cells is determined within 2 weeks following administration of the dose of TGFβ inhibitor, optionally at about 10 days following administration of the dose of TGFβ inhibitor.

163. The method of any one of embodiments 160-162, wherein the subject in step (i) or (ii) has not received previous cancer therapy, optionally wherein the subject in steps (i) and (ii) has not received previous cancer therapy.

164. The method of any one of embodiments 160-163, wherein the subject is to receive the cancer therapy if circulating MDSC levels are determined to be reduced.

165. The method of any one of embodiments 160-164, wherein the subject in step (i) or (ii) has received previous cancer therapy or is receiving cancer therapy, optionally wherein the subject in step (i) and (ii) has received previous cancer therapy or is receiving cancer therapy.

166. The method of embodiment 165, wherein the subject is to receive further cancer therapy if circulating MDSC levels are determined to be reduced.

167. The method of any one of embodiments 160-166, wherein the subject receives more than one dose of the TGFβ inhibitor prior to step (ii).

168. The method of any one of embodiments 160-167, wherein the sample is a whole blood sample or a blood component.

169. A cancer therapy agent for use in the treatment of cancer in a subject, wherein the subject has received a dose of a TGFβ inhibitor and wherein the circulating MDSC level in the subject measured after the administration of the TGFβ inhibitor has been determined to be reduced as compared to the circulating MDSC level measured in the subject prior to administering the dose of the TGFβ inhibitor, wherein optionally the circulating MDSC level is measured using an antibody that binds LRRC33.

170. A combination therapy comprising a dose of a TGFβ inhibitor and a cancer therapy agent for use in the treatment of cancer, wherein the treatment comprises simultaneous, separate, or sequential administration to a subject of a dose of the TGFβ inhibitor and the cancer therapy agent, and wherein the circulating MDSC level in the subject measured after the administration of the TGFβ inhibitor has been determined to be reduced as compared to the circulating MDSC level measured in the subject prior to administering the dose of the TGFβ inhibitor, wherein optionally the circulating MDSC level is measured using an antibody that binds LRRC33.

171. The combination therapy for use according to embodiment 170, wherein the subject has not received previous cancer therapy and wherein the circulating MDSC level in the subject has been determined to be reduced prior to administration of the cancer therapy agent.

172. The combination therapy for use according to embodiment 170 or embodiment 171, wherein the subject has not received previous cancer therapy, wherein the subject receives the TGFβ inhibitor prior to the cancer therapy agent.

173. The combination therapy for use according to embodiment 170, wherein the subject receives the cancer therapy agent prior to the TGFβ inhibitor.

174. A TGFβ inhibitor for use in the treatment of cancer in a subject, wherein the subject has received at least a first dose of the TGFβ inhibitor, and wherein the treatment comprises administering a further dose of the TGFβ inhibitor, provided that: the circulating MDSC level in the subject measured after the administration of the at least first dose of the TGFβ inhibitor is reduced as compared to the circulating MDSC level measured in the subject prior to administering a dose of the TGFβ inhibitor, wherein optionally the circulating MDSC level is measured using an antibody that binds LRRC33.

175. A TGFβ inhibitor for use in the treatment of cancer in a subject, wherein the subject is administered a dose of the TGFβ inhibitor, and wherein the TGFβ inhibitor reduces or reverses immune suppression in the cancer, wherein said reduced or reversed immune suppression has been determined by a reduction in the circulating MDSC level in the subject measured after the administration of the TGFβ inhibitor as compared to the circulating MDSC level measured in the subject prior to administering the dose of the TGFβ inhibitor, wherein optionally the circulating MDSC level is measured using an antibody that binds LRRC33.

176. The cancer therapy agent for use according to embodiment 169, the combination therapy for use according to embodiment 170 or any embodiment dependent thereon, or the TGFβ inhibitor for use according to embodiment 174 or embodiment 175, wherein the subject has received more than one dose of the TGFβ inhibitor prior to the determination that the circulating MDSC levels are reduced.

177. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the level of circulating MDSC cells is determined within 3-6 weeks following administration of the dose of TGFβ inhibitor, optionally within 3 weeks or at about 3 weeks, optionally within 2 weeks or at about 10 days, following administration of the dose of TGFβ inhibitor, optionally wherein said dose of TGFβ inhibitor is the first dose of the TGFβ inhibitor that the subject has received.

178. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has not received previous cancer therapy.

179. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject is receiving cancer therapy.

180. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject will be receiving cancer therapy.

181. The cancer therapy agent for use according to embodiment 169 or any embodiment dependent thereon, the combination therapy for use according to embodiment 170 or any embodiment dependent thereon, or the TGFβ inhibitor for use according to any one of embodiments 178-180, wherein the cancer therapy comprises immunotherapy, chemotherapy, radiation therapy, engineered immune cell therapy (e.g., CAR-T therapy), cancer vaccine therapy and/or oncolytic viral therapy.

182. The cancer therapy agent for use according to embodiment 169 or any embodiment dependent thereon, the combination therapy for use according to embodiment 170 or any embodiment dependent thereon, or the TGFβ inhibitor for use according to any one of embodiments 178-180, wherein the cancer therapy is immunotherapy comprising checkpoint inhibitor therapy, optionally wherein the checkpoint inhibitor comprises an agent targeting programmed cell death protein 1 (PD-1) or programmed cell death protein 1 ligand (PD-L1), optionally wherein the checkpoint inhibitor comprises an anti-PD-(L)1 antibody.

183. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the circulating MDSCs are G-MDSCs.

184. The method of determining therapeutic efficacy, the cancer therapy agent for use, the combination therapy for use, or TGFβ inhibitor for use according to embodiment 183, wherein the G-MDSCs express one or more of LOX-1+, CD11b+, HLADR−, CD14−, CD15+, CD33+/low, and CD66+.

185. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the circulating MDSC levels are reduced by at least 10%, optionally by at least 15%, 20%, 25%, or more.

186. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the circulating MDSC levels have been determined from a whole blood sample or a blood component obtained from the subject.

187. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the TGFβ inhibitor is a TGFβ1 inhibitor, optionally wherein the TGFβ inhibitor is a TGFβ1-specific inhibitor.

188. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has circulating MDSC levels at least 2-fold above circulating MDSC levels in a healthy subject prior to a treatment.

189. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has or is suspected of having cancer.

190. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the level of a tumor-associated immune cell in the subject measured after the administration of the first dose of the TGFβ inhibitor is changed as compared to the level of said tumor-associated immune cell in the subject measured prior to the administration of the first dose of the TGFβ inhibitor.

191. The method of determining therapeutic efficacy according to embodiment 160 or any embodiment dependent thereon, which further comprises:
(iv) determining the level of one or more tumor-associated immune cells in a sample obtained from the subject prior to administering the TGFβ inhibitor;
(v) determining the level of one or more tumor-associated immune cells in a sample obtained from the subject after the administration of the TGFβ inhibitor; and
(vi) determining whether the level determined in step (v) is changed compared to the level determined in step (iv), such change being indicative of therapeutic efficacy of the cancer treatment.

192. The method according to embodiment 191, wherein a change in level of one or more tumor-associated immune cells indicates reduction or reversal of immune suppression in the cancer.

193. The method according to embodiment 191 or embodiment 192, wherein the tumor-associated immune cells comprise CD8+ T cells and/or tumor-associated macrophages (TAMs).

194. The method according to any one of embodiments 191-193, wherein the change in the levels of one or more tumor-associated immune cells comprises at least a 10%, optionally at least a 15%, 20%, 25%, or more, increase in CD8+ T cell levels.

195. The method according to any one of embodiments 191-194, wherein the change in the levels of one or more tumor-associated immune cells comprises at least a 10%, optionally at least a 15%, 20%, 25%, or more, increase in the level of TAMs.

196. The method according to any one of embodiments 191-195, wherein the level of one or more tumor-associated immune cells is determined, in a sample obtained from the subject, by immunohistochemistry analysis.

197. The method according to any one of embodiments 191-196, wherein the level of one or more tumor-associated immune cells is determined by in vivo imaging.

198. The method according to any one of embodiments 191-197, wherein the sample is a tumor biopsy sample, wherein the tumor biopsy sample is optionally a core needle biopsy sample of the tumor.

199. The cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the level of circulating latent TGFβ (e.g., circulating latent TGFβ1) in the subject measured after the administration of the first dose of the TGFβ inhibitor is changed as compared to the level of said circulating latent TGFβ in the subject measured prior to the administration of the first dose of the TGFβ inhibitor.

200. The method of determining therapeutic efficacy according to embodiment 160 or embodiment 191 or any embodiment dependent thereon, which further comprises:
(vii) determining the level of circulating latent TGFβ in a sample obtained from the subject prior to administering the TGFβ inhibitor;
(viii) determining the level of circulating latent TGFβ in a sample obtained from the subject after the administration of the TGFβ inhibitor; and
(ix) determining whether the level determined in step (viii) is increased compared to the level determined in step (vii), such increase being indicative of therapeutic efficacy of the cancer treatment.

201. The method according to embodiment 200, wherein the level of circulating latent TGFβ is determined in a sample obtained from the subject and wherein the sample is a whole blood sample or a blood component.

202. The method according to embodiment 200, wherein the circulating latent TGFβ is circulating latent TGFβ1.

203. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has cancer that is resistant to a cancer therapy that does not comprise a TGFβ inhibitor.

204. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject is poorly responsive to a cancer therapy that does not comprise a TGFβ inhibitor.

205. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject is currently receiving or previously received a cancer therapy that does not comprise a TGFβ inhibitor.

206. The method of determining therapeutic efficacy according to embodiment 160 or any embodiment dependent thereon, the cancer therapy agent for use according to embodiment 169 or any embodiment dependent thereon, the combination therapy for use according to embodiment 170 or any embodiment dependent thereon, or the TGFβ inhibitor for use according to embodiment 174 or embodiment 175 or any embodiment dependent thereon, wherein the subject has cancer that is resistant to a cancer therapy that does not comprise a TGFβ inhibitor.

207. The method, the medical use, the cancer therapy agent for use, the TGFβ inhibitor for use, or the combination therapy for use according to any one of the preceding embodiments, wherein the TGFβ inhibitor is administered to the subject intravenously.

208. The method, the medical use, the cancer therapy agent for use, the TGFβ inhibitor for use, or the combination therapy for use according to any one of the preceding embodiments, wherein the TGFβ inhibitor is administered at a concentration of about 37.5 mg/kg, 30 mg/kg, 20 mg/kg, 10 mg/kg, 7 mg/kg, 6 mg/kg, 5 mg/kg, 4 mg/kg, 3 mg/kg, 2 mg/kg, 1 mg/kg, or less.

209. The method, the medical use, the cancer therapy agent for use, the TGFβ inhibitor for use, or the combination therapy for use according to any one of the preceding embodiments, wherein the TGFβ inhibitor is administered in an amount of about 3000 mg, 2400 mg, 1600 mg, 800 mg, 240 mg, 80 mg, or less.

210. The method, the medical use, the cancer therapy agent for use, the TGFβ inhibitor for use, or the combination therapy for use according to embodiment 204 or embodiment 205, wherein the TGFβ inhibitor is administered about every three weeks.

211. The method, the medical use, the cancer therapy agent for use, the TGFβ inhibitor for use, or the combination therapy for use according to any one of the preceding embodiments, wherein the cancer comprises an immune-excluded tumor.

212. The method, the medical use, the cancer therapy agent for use, the TGFβ inhibitor for use, or the combination therapy for use according to any one of the preceding embodiments, wherein the cancer is a myeloproliferative disorder, wherein optionally the myeloproliferative disorder is myelofibrosis.

213. The method, the medical use, the cancer therapy agent for use, the TGFβ inhibitor for use, or the combination therapy for use according to any one of the preceding embodiments, wherein the cancer is a highly metastatic cancer.

214. The method, the medical use, the cancer therapy agent for use, the TGFβ inhibitor for use, or the combination therapy for use according to any one of the preceding embodiments, wherein the cancer is colorectal cancer, lung cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer, or thyroid cancer.

215. The method, the medical use, the cancer therapy agent for use, the TGFβ inhibitor for use, or the combination therapy for use according to any one of the preceding embodiments, wherein the subject is at risk of developing aortic stenosis.

216. The method, the medical use, the cancer therapy agent for use, the TGFβ inhibitor for use, or the combination therapy for use according to any one of the preceding embodiments, wherein the cancer is TGFβ1-positive.

217. The method, the medical use, the cancer therapy agent for use, the TGFβ inhibitor for use, or the combination therapy for use according to any one of the preceding embodiments, wherein the cancer co-expresses TGFβ1 and TGFβ3.

218. The method, the medical use, the cancer therapy agent for use, the TGFβ inhibitor for use, or the combination therapy for use according to any one of the preceding embodiments, wherein the tumor is a TGFβ1-dominant tumor.

219. A method for manufacturing a pharmaceutical composition comprising a TGFβ inhibitor, the method comprising the steps of:
i) providing a TGFβ inhibitor that meets the following criteria:
   a) the TGFβ inhibitor is a monoclonal antibody, an antigen-binding fragment thereof, or a multispecific construct that is capable of binding a TGFβ;
   b) the TGFβ inhibitor binds the TGFβ with a K_{D} of <1.0 nM, preferably K_{D}<500 pM, as measured by a SPR-based assay (e.g., Biacore) and inhibits TGFβ1;
   c) the TGFβ inhibitor is effective in vivo in a preclinical model at a dose that does not cause a toxicity associated with pan-inhibition of TGFβ when dosed with at least 10 times the minimum efficacious amount for at least 4 weeks in an animal model;
ii) carrying out an immune safety assessment comprising:
   a) a cytokine release assay (in vitro and/or in vivo); and/or,
   b) a platelet assay
iii) producing the TGFβ inhibitor at a scale of 250L or larger; and,
iv) formulating the TGFβ inhibitor into a pharmaceutical composition with one or more excipients.

220. The method of embodiment 219, wherein the TGFβ is TGFβ1.

221. The method of embodiment 219, wherein the TGFβ is a proTGFβ complex, mature TGFβ growth factor, or a ligand-binding domain of a TGFβ receptor.

222. The method of embodiment 219, wherein the TGFβ inhibitor is effective in causing tumor growth regression, prolonged survival, and/or normalized gene expression of PAI-1, CCL2, FN-1, ACTA2, Col1a1, Col3a1, FN-1, CTGF, and/or TGFβ1.

223. The method of embodiment 222, wherein the tumor is a TGFβ1-dominant tumor, wherein optionally the tumor further expresses TGFβ3.

224. The method of embodiment 219 wherein the toxicity associated with pan-inhibition of TGFβ comprises one or more of a cardiovascular toxicity (e.g., a valvulopathy), epithelial hyperplasia, bleeding, and skin lesion.

225. The method of embodiment 219, wherein the immune safety assessment comprises an in vitro cytokine release assay.

226. The method of embodiment 219, wherein the scale of the production is at least 500L, at least 1000L, at least 2000L.

227. The method of any one of embodiments 219 to 226, wherein the production comprises a eukaryotic cell culture, wherein optionally the eukaryotic cell culture is a mammalian cell culture, plant cell culture, or an insect cell culture.

228. The method of embodiment 227, wherein the mammalian cell culture comprises a CHO cell, MDCK cell, NSO cell, Sp2/0 cell, BHK cell, Murine C127 cell, Vero cell, HEK293 cell, HT-1080 cell, or PER.C6 cell.

229. A method of treating a TGFβ-related disorder in a subject, the method comprising administering to the subject a therapeutically effective amount of a TGFβ inhibitor to treat the disorder, wherein the therapeutically effective amount is an amount sufficient to increase the level of circulating latent TGFβ after the administration.

230. A method of treating a TGFβ-related disorder in a subject, the method comprising administering a TGFβ inhibitor and monitoring levels of circulating latent TGFβ after administration.

231. The method of embodiments 229 or 230, wherein the TGFβ-related disorder is a TGFβ1-related disorder.

232. The method of embodiment 231, wherein the TGFβ1-related disorder is a cancer.

233. The method of embodiment 231, wherein the TGFβ1-related disorder is an immune disorder

234. The method of any one of embodiments 229-233, wherein if the level of circulating latent TGFβ after the administration of the TGFβ inhibitor is increased, e.g., by at least 2-fold, 3-fold, 4-fold, 5-fold, 6-fold, 7-fold, 8-fold, 9-fold, 10-fold, or more, relative to the level prior to the administration, an additional dose of the TGFβ inhibitor is administered.

235. The method of any one of embodiments 229-234, wherein the level of circulating latent TGFβ after the administration of the TGFβ inhibitor is increased to a maximum of at least 1000 μg/ml.

236. The method of any one of embodiments 229-235, wherein the level of circulating latent TGFβ after the administration of the TGFβ inhibitor is increased to a maximum of about 1000 μg/ml to about 8000 μg/ml.

237. The method of any one of embodiments 229-236, wherein the level of circulating latent TGFβ after the administration of the TGFβ inhibitor is increased to a maximum about 2000 μg/ml to about 6500 μg/ml.

238. The method of any one of 229-237, wherein the level of circulating latent TGFβ after the administration of the TGFβ inhibitor is increased by a minimum of about 1.5-fold.

239. The method of any one of embodiments 229-238, wherein the level of circulating latent TGFβ is measured about 8 to about 672 hours following administration of the TGFβ inhibitor.

240. The method of any one of embodiments 229-239, wherein the level of circulating latent TGFβ is measured about 24 hours to about 336 hours following administration of the TGFβ inhibitor.

241. The method of any one of embodiments 229-240, wherein the level of circulating latent TGFβ is measured about 72 hours to about 240 hours following administration of the TGFβ inhibitor.

242. The method of any one of embodiments 229-241, wherein the TGFβ inhibitor is administered at a dose of about 1 mg/kg to about 30 mg/kg.

243. The method of any one of embodiments 229-242, wherein the TGFβ inhibitor is administered at a dose of about 5 mg/kg to about 20 mg/kg.

244. The method of any one of embodiments 229-243, wherein the TGFβ inhibitor is administered at a dose of about 2 mg/kg to about 7 mg/kg.

245. The method of any one of embodiments 229-245, wherein the TGFβ inhibitor is administered about every three weeks.

246. A method of determining the efficacy of a cancer treatment in a subject, comprising determining the level of circulating latent TGFβ1 in a first sample from the subject, administering a dose of a TGFβ1 inhibitor to the subject, and determining the level of circulating latent TGFβ in a second sample from the subject after administration, wherein an increase of at least 2-fold, 3-fold, 4-fold, 5-fold, 6-fold, 7-fold, 8-fold, 9-fold, 10-fold, or more in circulating latent TGFβ levels between the first sample and the second sample indicates treatment efficacy.

247. A method of treating a subject with a solid cancer, comprising determining the level of circulating latent TGFβ1 in a first sample from the subject, administering to the subject a dose of a TGFβ1 inhibitor, and determining the level of circulating latent TGFβ in a second sample from the subject after administration.

248. The method of embodiment 247, wherein if the subject has a ratio of circulating latent TGFβ after the administration to before the administration of at least 1.2, an additional dose of the TGFβ inhibitor is administered.

249. The method of any one of embodiments 246-248, wherein the second sample is collected from the subject 24 hours to 56 days after the administration.

250. The method of any one of embodiments 229-249, wherein the TGFβ inhibitor is a TGFβ activation inhibitor, e.g., a TGFβ1-selective inhibitor.

251. The method of embodiment 2250, wherein the TGFβ inhibitor is Ab6.

252. The method of any one of embodiments 229-251, wherein the therapeutically effective amount of the TGFβ inhibitor is between 0.1 mg/kg to 30 mg/kg per dose.

253. The method of any one of embodiments 229-252, wherein the therapeutically effective amount of the TGFβ inhibitor is between 1 mg/kg and 10 mg/kg per dose.

254. The method of any one of embodiments 229-253, wherein the therapeutically effective amount of the TGFβ inhibitor is between 2 mg/kg and 7 mg/kg per dose.

255. The method of any one of embodiments 229-254, wherein the TGFβ inhibitor is dosed weekly, every 2 weeks, every 3 weeks, every 4 weeks, monthly, every 6 weeks, every 8 weeks, bimonthly, every 10 weeks, every 12 weeks, every 3 months, every 4 months, every 6 months, every 8 months, every 10 months, or once a year.

256. The method of any one of embodiments 229-255, wherein the TGFβ inhibitor is dosed about every 3 weeks.

257. The method of any one of embodiments 229-256, wherein the TGFβ inhibitor is administered intravenously or subcutaneously.

258. The method of any one of embodiments 229-257, wherein the latent TGFβ is latent TGFβ1. 259. The method of any one of embodiments 229-258, wherein the level of circulating latent TGFβ is measured in a blood sample.

260. The method of any one of embodiments 229-259, wherein the blood sample is a serum sample or a plasma sample.

261. The method of any one of embodiments 229-260, wherein the circulating latent TGFβ levels are measured by ELISA.

262. The method of any one of embodiments 229-261, further comprising determining the levels of circulating MDSCs in the subject prior to and after administration of the TGFβ inhibitor, wherein optionally the circulating MDSC levels are determined with the use of an antibody that binds LRRC33.

263. The method of embodiment 262, wherein a reduction in the levels of circulating MDSCs after the administration as compared to before the administration indicates therapeutic efficacy and, optionally, one or more additional treatments comprising the TGFβ inhibitor is administered.

264. The method of embodiment 262, wherein the circulating MDSCs are G-MDSCs.

265. The method of embodiment 264, wherein the G-MDSCs express one or more of CD11 b, CD33, CD15, LOX-1, CD66b, and HLA-DR^{lo/-}.

266. The method of any one of embodiments 243-246, wherein the circulating MDSC levels are determined from whole blood or a blood component collected from the subject.

267. The method of any one of embodiments 262-266, wherein administration of the TGFβ inhibitor reduces circulating MDSC levels by at least 10%, optionally by at least 15%, 20%, 25%, or more.

268. The method of any one of embodiments 262-267, wherein circulating latent TGFβ levels are increased by at least 50% and circulating MDSC levels are decreased by at least 15%, 20%, 25%, or more.

269. The method, the medical use, the cancer therapy agent for use, the TGFβ inhibitor for use, or the combination therapy for use according to any one of the preceding embodiments, wherein the TGFβ inhibitor inhibits TGFβ1 signaling.

270. The method, the medical use, the cancer therapy agent for use, the TGFβ inhibitor for use, or the combination therapy for use according to any one of the preceding embodiments, wherein the TGFβ inhibitor Inhibits TGFβ1 signaling but does not inhibit TGFβ3 signaling at a therapeutically effective dose.

271. The method, the medical use, the cancer therapy agent for use, the TGFβ inhibitor for use, or the combination therapy for use according to any one of the preceding embodiments, wherein the TGFβ inhibitor does not inhibit TGFβ2 signaling and TGFβ3 signaling at a therapeutically effective dose.

272. The method, the medical use, the cancer therapy agent for use, the TGFβ inhibitor for use, or the combination therapy for use according to any one of the preceding embodiments, wherein the TGFβ inhibitor does not bind to free TGFβ growth hormones.

273. The method, the medical use, the cancer therapy agent for use, the TGFβ inhibitor for use, or the combination therapy for use according to any one of the preceding embodiments, wherein the TGFβ inhibitor binds to pro- and/or -latent TGFβ1.

274. The method, the medical use, the cancer therapy agent for use, the TGFβ inhibitor for use, or the combination therapy for use according to any one of the preceding embodiments, wherein the TGFβ inhibitor binds to at least a portion of a Latency Lasso in TGFβ1.

275. The method, the medical use, the cancer therapy agent for use, the TGFβ inhibitor for use, or the combination therapy for use according to any one of the preceding embodiments, wherein the TGFβ inhibitor binds to at least a portion of a Finger-1 domain in TGFβ1.

276. The method, the medical use, the cancer therapy agent for use, the TGFβ inhibitor for use, or the combination therapy for use according to any one of the preceding embodiments, wherein the TGFβ inhibitor is a neutralizing antibody or a ligand trap.

277. The method, the medical use, the cancer therapy agent for use, the TGFβ inhibitor for use, or the combination therapy for use according to any one of the preceding embodiments, wherein the TGFβ inhibitor binds selectively to TGFβ1, optionally selectively to a pro- and/or latent-TGFβ1.

278. The method, the medical use, the cancer therapy agent for use, the TGFβ inhibitor for use, or the combination therapy for use according to any one of the preceding embodiments, wherein the TGFβ inhibitor is an isolated antibody or antigen-binding fragment thereof which is capable of specifically binding a proTGFβ1 complex at (i) a first binding region comprising at least a portion of Latency Lasso (SEQ ID NO: 126); and ii) a second binding region comprising at least a portion of Finger-1 (SEQ ID NO: 124).

279. The method, the medical use, the cancer therapy agent for use, the TGFβ inhibitor for use, or the combination therapy for use according to embodiment 278, wherein the first binding region further comprises an amino acid sequence of SEQ ID NO: 134 or a portion thereof.

280. The method, the medical use, the cancer therapy agent for use, the TGFβ inhibitor for use, or the combination therapy for use according to embodiment 278, wherein the second binding region further comprises an amino acid sequence of SEQ ID NO: 143 or a portion thereof.

281. The method, the medical use, the cancer therapy agent for use, the TGFβ inhibitor for use, or the combination therapy for use according to any one of the preceding embodiments, wherein the TGFβ inhibitor comprises an isolated antibody or antigen-binding fragment thereof, comprising three heavy chain complementarity determining regions comprising amino acid sequences of SEQ ID NO: 1 (H-CDR1), SEQ ID NO: 2 (H-CDR2), and SEQ ID NO: 3 (H-CDR3), and three light chain complementarity determining regions comprising amino acid sequences of SEQ ID NO: 4 (L-CDR1), SEQ ID NO: 5 (L-CDR2), and SEQ ID NO: 6 (L-CDR3), as defined by the IMTG numbering system.

282. The method, the medical use, the cancer therapy agent for use, the TGFβ inhibitor for use, or the combination therapy for use according to any one of the preceding embodiments, wherein the TGFβ inhibitor comprises an isolated antibody or antigen-binding fragment thereof, comprising three heavy chain complementarity determining regions (H-CDR1, H-CDR2, and H-CDR3) from a heavy chain variable region comprising an amino acid sequence of SEQ ID NO: 7, and three light chain complementarity determining regions (L-CDR1, L-CDR2, and L-CDR3) from a light chain variable region comprising an amino acid sequence of SEQ ID NO: 8.

283. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising three heavy chain complementarity determining regions (H-CDR1, H-CDR2, and H-CDR3) from a heavy chain variable region that is at least 90% identical to an amino acid sequence of SEQ ID NO: 7, and three light chain complementarity determining regions (L-CDR1, L-CDR2, and L-CDR3) from a light chain variable region that is at least 90% identical to an amino acid sequence of SEQ ID NO: 8.

284. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising a heavy chain variable region that is at least 90% identical to an amino acid sequence of SEQ ID NO: 7 and a light chain variable region that is at least 90% identical to an amino acid sequence of SEQ ID NO: 8.

285. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising a heavy chain variable region comprising an amino acid sequence of SEQ ID NO: 7 and a light chain variable region comprising an amino acid sequence of SEQ ID NO: 8.

286. The method, the medical use, the cancer therapy agent for use, the TGFβ inhibitor for use, or the combination therapy for use according to any of the preceding embodiments, wherein the TGFβ inhibitor comprises an isolated antibody or antigen-binding fragment thereof, comprising a heavy chain comprising an amino acid sequence of SEQ ID NO: 9 and a light chain comprising an amino acid sequence of SEQ ID NO: 11.

287. The method, the medical use, the cancer therapy agent for use, the TGFβ inhibitor for use, or the combination therapy for use according to any of the preceding embodiments, wherein the TGFβ inhibitor cross-blocks and/or competes for binding to TGFβ1 with an antibody or antigen-binding fragment comprising a heavy chain variable domain of SEQ ID NO: 7, and a light chain variable domain of SEQ ID NO: 8.

288. The method, the medical use, the cancer therapy agent for use, the TGFβ inhibitor for use, or the combination therapy for use according to any one of the preceding embodiments, wherein the TGFβ inhibitor is a monoclonal antibody, optionally a fully human or humanized antibody, or an antigen binding fragment thereof.

289. The method, the medical use, the cancer therapy agent for use, the TGFβ inhibitor for use, or the combination therapy for use according to any one of the preceding embodiments, wherein the TGFβ inhibitor is present in a multispecific or bispecific construct.

290. The method, the medical use, the cancer therapy agent for use, the TGFβ inhibitor for use, or the combination therapy for use according to embodiment 289, wherein the multispecific or bispecific construct is also capable of binding to an immune cell-surface antigen,

291. The method, the medical use, the cancer therapy agent for use, the TGFβ inhibitor for use, or the combination therapy for use according to embodiment 290, wherein the immune cell-surface antigen is PD-1, PD-L1, CTLA4, or LAG3.

292. The method, the medical use, the cancer therapy agent for use, the TGFβ inhibitor for use, or the combination therapy for use according to embodiment 290 or 291, wherein the immune cell-surface antigen is PD-1 or PD-L1, optionally comprising an anti-PD-1 or anti-PD-L1 antibody or antigen binding fragment thereof.

293. The method, the medical use, the cancer therapy agent for use, the TGFβ inhibitor for use, or the combination therapy for use according to any of the preceding embodiments, wherein the TGFβ inhibitor comprises a human IgG₄ or IgG₁ constant region.

294. The method, the medical use, the cancer therapy agent for use, the TGFβ inhibitor for use, or the combination therapy for use according to any of the preceding embodiments, wherein the subject is a human patient and wherein the patient has a historically documented solid tumor that is metastatic or locally advanced, for which standard-of-care therapy does not exist, has failed in the patient, or is not tolerated by the patient, or for which the patient has been assessed as not suitable candidate or otherwise ineligible for the standard-of-care therapy.

295. The method, the medical use, the cancer therapy agent for use, the TGFβ inhibitor for use, or the combination therapy for use according to any of the preceding embodiments, wherein the subject is a human patient and wherein the patient has a history of primary anti-PD-(L)1 antibody nonresponse presenting either as progressive disease or stable disease (e.g., not improving, but also not worsening, clinically or radiographically) after at least 3 cycles of treatment with an anti-PD-(L)1 antibody therapy (optionally alone or in combination with chemotherapy) approved for that tumor type.

296. The method, the medical use, the cancer therapy agent for use, the TGFβ inhibitor for use, or the combination therapy for use according to any of the preceding embodiments, wherein the subject is a human patient and wherein the patient has received the most recent dose of anti-PD-(L)1 antibody therapy within 6 months of the administration of the TGFβ inhibitor.

297. The method, the medical use, the cancer therapy agent for use, the TGFβ inhibitor for use, or the combination therapy for use according to any of the preceding embodiments, wherein the subject is a human patient and wherein the patient has NSCLC and has genomic tumor aberrations for which a targeted therapy is available (wherein optionally the targeted therapy targets anaplastic lymphoma kinase and/or EGFR), wherein further optionally the patient has progressed on an approved therapy for these aberrations or did not tolerate an approved therapy for these aberrations, or was not considered suitable candidates or was otherwise ineligible for an approved therapy for these aberrations.

298. The method, the medical use, the cancer therapy agent for use, the TGFβ inhibitor for use, or the combination therapy for use according to any of the preceding embodiments, wherein the subject is a human patient and wherein the patient has measurable disease as determined by Response Evaluation Criteria in Solid Tumor (RECIST) v1.1.

299. The method, the medical use, the cancer therapy agent for use, the TGFβ inhibitor for use, or the combination therapy for use according to any of the preceding embodiments, wherein the subject is a human patient and wherein the patient has an Eastern Cooperative Oncology Group performance status (PS) 0-1.

300. The method, the medical use, the cancer therapy agent for use, the TGFβ inhibitor for use, or the combination therapy for use according to any of the preceding embodiments, wherein the subject is a human patient and wherein the patient has a predicted life expectancy of ≥3 months.

301. The method of embodiment 1, wherein the reduced circulating MDSCs are M-MDSCs.

302. The method of embodiment 1 or embodiment 2, wherein the M-MDSCs express one or more of CD11b+, HLADR−/low, CD14+, CD15−, CD33+/high, and CD66b−.

303. The composition, composition for use, or method of any one of the preceding embodiments, wherein the TGFβ inhibitor is shown to cause no significant adverse events (e.g., dose-limiting toxicities) in a preclinical animal model when dosed at up to 100, 200, or 300 μg/kg weekly for 4 weeks, 8 weeks or up to12 weeks, as assessed by standard toxicology analyses or according to the present disclosure.

304. The composition for use, the TGFβ inhibitor for use, or the method according to any one of the preceding embodiments, wherein selection of the composition or the TGFβ inhibitor comprises in vivo efficacy and safety criteria, wherein the safety criteria includes: i) lack of platelet aggregation, activation and/or binding when assessed under the condition according to the present disclosure, and, ii) lack of significant (e.g., within 2.5-fold of control) cytokine release, when assessed under the condition according to the present disclosure.

305. The composition for use, the TGFβ inhibitor for use, or the method according to any one of the preceding embodiments, wherein the pharmacodynamics of the TGFβ inhibitor are assessed by measuring circulatory latent TGFβ1 levels before and after the administration of the TGFβ1 inhibitor in blood (serum) samples collected from the subject.

### LIST OF CERTAIN OTHER EMBODIMENTS

1. A TGFβ inhibitor for use in the treatment of a cancer in a subject, wherein the treatment comprises determining a level of circulating TGFβ in a blood sample collected from the subject and administering the TGFβ inhibitor to the subject, wherein the circulating TGFβ level is determined or has been determined by processing the blood sample below room temperature in a sample tube comprising an anticoagulant.

2. A method of treating a cancer in a subject, comprising:
(i) determining a level of circulating TGFβ in the subject prior to administering a TGFβ inhibitor;
(ii) administering to the subject a therapeutically effective amount of the TGFβ inhibitor;
(iii) determining a level of circulating TGFβ in the subject after administration; and
wherein the circulating TGFβ level is determined or has been determined by processing a blood sample from the subject below room temperature in a sample tube comprising an anticoagulant.

3. A method of determining therapeutic efficacy in a subject being treated for a cancer, comprising:
(i) determining a level of circulating TGFβ in the subject prior to administering a TGFβ inhibitor;
(ii) administering to the subject a therapeutically effective amount of the TGFβ inhibitor; and
(iii) determining a level of circulating TGFβ in the subject after administration;
wherein an increase in circulating TGFβ levels after administration as compared to before administration indicates therapeutic efficacy, and wherein the circulating TGFβ level is determined or has been determined by processing a blood sample from the subject below room temperature in a sample tube comprising an anticoagulant.

4. The method of embodiment 3, wherein an increase of at least 1.5-fold, at least 2-fold, at least 2.5-fold, at least 3-fold, at least 4-fold, at least 5-fold, or more in circulating TGFβ levels after administration as compared to before administration indicates therapeutic efficacy.

5. A method of determining targeting engagement in a subject having cancer, comprising:
(i) determining a level of circulating TGFβ in the subject prior to administering a TGFβ inhibitor;
(ii) administering to the subject a therapeutically effective amount of the TGFβ inhibitor;
(iii) determining a level of circulating TGFβ in the subject after administration; and
wherein an increase in circulating TGFβ levels after administration as compared to before administration indicates target engagement of the TGFβ inhibitor, and wherein a circulating TGFβ level is determined or has been determined by processing a blood sample from the subject below room temperature in a sample tube comprising an anticoagulant.

6. The method of embodiment 4, wherein an increase of at least 1.5-fold, at least 2-fold, at least 2.5-fold, at least 3-fold, at least 4-fold, at least 5-fold, or more in circulating TGFβ levels after administration as compared to before administration indicates target engagement of the TGFβ inhibitor.

7. The method of any one of embodiments 2-6, wherein the administration of the TGFβ inhibitor is continued if the level of circulating TGFβ after the administration is increased by at least 1.5-fold, at least 2-fold, at least 2.5-fold, at least 3-fold, at least 4-fold, at least 5-fold, or more as compared to the level of circulating TGFβ before the administration.

8. A method of treating a cancer in a subject, comprising administering a second dose of a TGFβ inhibitor to a subject having an elevated level of circulating TGFβ after receiving a first dose the TGFβ inhibitor, wherein the level of TGFβ has been measured by processing a blood sample from the subject below room temperature in a sample tube comprising an anticoagulant.

9. A method of treating a cancer in a subject, comprising:
(i) determining a level of circulating TGFβ in the subject prior to administering a TGFβ inhibitor;
(ii) administering to the subject a first dose of TGFβ inhibitor;
(iii) determining a level of circulating TGFβ in the subject after administration; and
(iv) administering a second dose of the TGFβ inhibitor to the subject if the level of circulating TGFβ is elevated,
wherein determining the level of TGFβ comprises processing a blood sample from the subject below room temperature in a sample tube comprising an anticoagulant.

10. The method of embodiment 8 or 9, wherein the level of circulating TGFβ after the first dose of the TGFβ inhibitor is elevated by at least 1.5-fold, at least 2-fold, at least 2.5-fold, at least 3-fold, at least 4-fold, at least 5-fold, or more as compared the level of circulating TGFβ before the first dose of the TGFβ inhibitor.

11. The TGFβ inhibitor or method of any one of embodiments 1-10, wherein the cancer comprises a solid tumor, wherein optionally the solid tumor is selected from: melanoma (e.g., metastatic melanoma), renal cell carcinoma, triple-negative breast cancer, HER2-positive breast cancer, colorectal cancer (e.g., microsatellite stable-colorectal cancer), lung cancer (e.g., metastatic non-small cell lung cancer, small cell lung cancer), esophageal cancer, pancreatic cancer, bladder cancer, kidney cancer (e.g., transitional cell carcinoma, renal sarcoma, and renal cell carcinoma (RCC), including clear cell RCC, papillary RCC, chromophobe RCC, collecting duct RCC, or unclassified RCC, uterine cancer, prostate cancer, stomach cancer (e.g., gastric cancer), head and neck squamous cell cancer, urothelial carcinoma, hepatocellular carcinoma, or thyroid cancer.

12. The TGFβ inhibitor or method of any one of embodiments 1-11, comprising administering a checkpoint inhibitor therapy concurrently (e.g., simultaneously), separately, or sequentially with the TGFβ inhibitor, wherein the checkpoint inhibitor is optionally an anti-PD-1 antibody, anti-PD-L1 antibody, anti-CTLA-4-antibody, anti-LAG3 antibody, or an antigen-binding fragment thereof.

13. The TGFβ inhibitor or method of embodiment 12, wherein the cancer has an immune excluded phenotype, e.g., characterized by containing less than 5% intratumor CD8+ cells and greater than 5% margin CD8+ cells as assessed by an immunohistochemistry analysis capable of detecting individual tumor nest(s) within the tumor.

14. The TGFβ inhibitor or method of embodiment 13, wherein the cancer is characterized by having greater than 50% tumor area comprising tumor nest(s) comprising lower levels of CD8+ cells inside the tumor nest(s) relative to levels of CD8+ cells outside of the tumor nest, e.g., less than 5% CD8+ cells inside the tumor nest(s) and greater than 5% CD8+ cells outside the tumor nest(s), e.g., in the margin.

15. The TGFβ inhibitor or method of any one of embodiments 1-14, wherein processing the blood samples comprises one or more centrifugation steps at a speed of greater than 100×g and/or one or more centrifugation steps at a speed of below 15000×g.

16. The TGFβ inhibitor or method of any one of embodiments 1-15, wherein processing the blood samples comprises a centrifugation protocol comprising:
i) a first step of 5-25 minutes at 100-500×g and a second step of 10-40 minutes at 1000-5000×g; or
ii) a first step of 5-25 minutes at 1000-5000×g and a second step of 10-40 minutes at 1000-5000×g; or
iii) a first step of 5-25 minutes at 500-3000×g and a second step of 2-10 minutes at 7500-15000×g.

17. The TGFβ inhibitor or method of any one of embodiments 1-16, wherein processing the blood samples comprises a centrifugation protocol comprising:
i) a first step of 10 minutes at 150×g and a second step of 20 minutes at 2500×g; or
ii) a first step of 10 minutes at 2500×g and a second step of 20 minutes at 2500×g; or
iii) a first step of 10 minutes at 1500×g and a second step of 5 minutes at 12000×g.

18. The TGFβ inhibitor or method of any one of embodiments 1-17, wherein processing the blood samples comprises a centrifugation protocol comprising a first step of 10 minutes at 2500×g and a second step of 20 minutes at 2500×g.

19. The TGFβ inhibitor or method of any one of embodiments 1-18, wherein the anticoagulant comprises 0.1-0.5 M buffered trisodium citrate.

20. The TGFβ inhibitor or method of any one of embodiments 1-19, wherein the anticoagulant comprises 10-20 M theophylline.

21. The TGFβ inhibitor or method of any one of embodiments 1-20, wherein the anticoagulant comprises 2-5 M adenosine.

22. The TGFβ inhibitor or method of any one of embodiments 1-21, wherein the anticoagulant comprises 0.1-0.25 M dipyridamole.

23. The TGFβ inhibitor or method of any one of embodiments 1-22, wherein the anticoagulant comprises a citrate-theophylline-adenosine-dipyridamole (CTAD) solution, optionally comprising about 0.11 M buffered trisodium citrate solution, about 15 M theophylline, about 3.7 M adenosine, and about 0.198 M dipyridamole.

24. The TGFβ inhibitor or method of any one of embodiments 1-23, wherein the collection tube is coated with a solution that has a pH of 4.0-6.0, e.g., about 5.0.

25. The TGFβ inhibitor or method of any one of embodiments 1-24, wherein the collection tube is glass or plastic.

26. The TGFβ inhibitor or method of any one of embodiments 1-25, wherein the collection tube comprises a silicone coating.

27. The TGFβ inhibitor or method of any one of embodiments 1-26, wherein the collection tube has a Hemogard™ closure.

28. The TGFβ inhibitor or method of any one of embodiments 1-27, wherein the collection tube has a volume capacity of 2-3 mL, e.g., 2.7 mL.

29. The TGFβ inhibitor or method of any one of embodiments 1-28, wherein the collection tube is sterile.

30. The TGFβ inhibitor or method of any one of embodiments 1-29, wherein the collection tube is a BD Vacutainer™ CTAD blood collection tube.

31. The TGFβ inhibitor or method of any one of embodiments 1-30, wherein the blood sample is processed at 2-8° C. 32. The TGFβ inhibitor or method of any one of embodiments 1-31, wherein the blood sample is processed at 4° C.

33. The TGFβ inhibitor or method of any one of embodiments 1-32, wherein processing the blood sample comprises determining a level of platelet factor 4 (PF4) in the same sample from which the circulating TGFβ is determined, wherein the sample is used for measuring optionally the level of circulating TGFβ in a sample is normalized based on the PF4 levels determined from the same sample if the PF4 level is 500 ng/ml or less.

34. The TGFβ inhibitor or method of any one of embodiments 1-33, wherein the circulating TGFβ is circulating TGFβ1.

35. The TGFβ inhibitor or method of any one of embodiments 1-34, wherein the circulating TGFβ is circulating latent TGFβ1.

36. The TGFβ inhibitor or method of any one of embodiments 1-35, wherein the TGFβ inhibitor is administered at a dose of 2000 mg or 3000 mg and a frequency of once every two weeks, three weeks, or any multiples of two weeks or three weeks.

37. A method of treating a cancer in a human subject comprising administering to the subject a 2000 mg or 3000 mg dose of a TGFβ inhibitor at a frequency of once every two weeks, three weeks, or any multiples of two weeks or three weeks, wherein the subject is not receiving a checkpoint inhibitor therapy.

38. A TGFβ inhibitor for use in the treatment of a cancer in a human subject, wherein the treatment comprises administering to the subject a 2000 mg or 3000 mg dose of a TGFβ inhibitor at a frequency of once every two weeks, three weeks, or any multiples of two weeks or three weeks, wherein the subject is not receiving a checkpoint inhibitor therapy.

39. A method of treating a cancer in a subject comprising administering to the subject a TGFβ inhibitor and a checkpoint inhibitor therapy, where the TGFβ inhibitor is administered at a dose of 2000 mg or 3000 mg and a frequency of once every two weeks, three weeks, or any multiples of two weeks or three weeks.

40. A TGFβ inhibitor for use in the treatment of a cancer in a subject, wherein the treatment comprises administering to the subject a TGFβ inhibitor and a checkpoint inhibitor therapy, where the TGFβ inhibitor is administered at a dose of 2000 mg or 3000 mg and a frequency of once every two weeks, three weeks, or any multiples of two weeks or three weeks.

41. The method or TGFβ inhibitor for use of any one of embodiments 37-40, wherein the subject is a non-responder to checkpoint inhibitor therapy.

42. The method or TGFβ inhibitor for use of any one of embodiments 37-41, wherein the subject has not received a checkpoint inhibitor therapy previously.

43. The method or TGFβ inhibitor for use of any one of embodiments 37-42, wherein the checkpoint inhibitor therapy is pembrolizumab, nivolumab, cemiplimab, atezolizumab, avelumab, budigalimab, or durvalumab.

44. The method or TGFβ inhibitor for use of any one of embodiments 37-43, wherein the TGFβ inhibitor is administered at a frequency of once every two weeks.

45. The method or TGFβ inhibitor for use of any one of embodiments 37-43, wherein the TGFβ inhibitor is administered at a frequency of once every three weeks.

46. The method or TGFβ inhibitor for use of any one of embodiments 37-43, wherein the TGFβ inhibitor is administered at a frequency of once every four weeks.

47. The method or TGFβ inhibitor for use of any one of embodiments 37-43, wherein the TGFβ inhibitor is administered at a frequency of once every six weeks.

48. The method or TGFβ inhibitor for use of any one of embodiments 37-47, wherein the TGFβ inhibitor is administered at a dose of 2000 mg.

49. The method or TGFβ inhibitor for use of any one of embodiments 37-47, wherein the TGFβ inhibitor is administered at a dose of 3000 mg.

50. The method or TGFβ inhibitor for use of any one of embodiments 37-49, wherein the TGFβ inhibitor is administered consecutively or concurrently with a checkpoint inhibitor, wherein the checkpoint inhibitor is optionally an anti-PD-1 antibody, anti-PD-L1 antibody, anti-CTLA-4-antibody, anti-LAG3 antibody, or an antigen-binding fragment thereof.

51. The method or TGFβ inhibitor for use of embodiment 50, wherein the TGFβ inhibitor is administered at the same frequency as the checkpoint inhibitor therapy.

52. A method of determining therapeutic efficacy in a subject being treated for a cancer, comprising:
(i) determining a level of P-Smad2 nuclear translocation in a tumor sample obtained from the subject prior to administering a TGFβ inhibitor;
(ii) administering to the subject one or more doses of the TGFβ inhibitor; and
(iii) determining a level of P-Smad2 nuclear translocation in a tumor sample obtained from the subject after the administration;
wherein a decrease in P-Smad2 nuclear translocation after the administration as compared to before the administration indicates therapeutic efficacy.

53. The method of embodiment 52, wherein a decrease of at least 1.3-fold, at least 1.5-fold, at least 2-fold, at least 3-fold, at least 4-fold, at least 5-fold, or more in P-Smad2 nuclear translocation levels after the administration as compared to before the administration indicates therapeutic efficacy.

54. A method of determining targeting engagement in a subject having cancer, comprising:
(i) determining a level of P-Smad2 nuclear translocation in a tumor sample obtained from the subject prior to administering a TGFβ inhibitor;
(ii) administering to the subject one or more doses of the TGFβ inhibitor;
(iii) determining a level of P-Smad2 nuclear translocation in a tumor sample obtained from the subject after the administration; and
wherein a decrease in P-Smad2 nuclear translocation after the administration as compared to before the administration indicates target engagement of the TGFβ inhibitor.

55. The method of embodiment 54, wherein a decrease of at least 1.3-fold, at least 1.5-fold, at least 2-fold, at least 3-fold, at least 4-fold, at least 5-fold, or more in P-Smad2 nuclear translocation levels after the administration as compared to before the administration indicates target engagement.

56. A method of treating cancer in a subject, comprising:
(i) determining a level of P-Smad2 nuclear translocation in a tumor sample obtained from the subject prior to administering a TGFβ inhibitor;
(ii) administering to the subject a first dose of the TGFβ inhibitor;
(iii) determining a level of P-Smad2 nuclear translocation in a tumor sample obtained from the subject after the administration; and
(iv) administering to the subject one or more additional doses of the TGFβ inhibitor if the P-Smad2 nuclear translocation after the administration of the first dose is decreased as compared to the P-Smad2 nuclear translocation before the administration of the first dose.

57. The method of embodiment 56, wherein P-Smad2 nuclear translocation is decreased by at least 1.3-fold, at least 1.5-fold, at least 2-fold, at least 3-fold, at least 4-fold, at least 5-fold, or more.

58. The method or TGFβ inhibitor for use according to any one of embodiments 54-57, wherein the determination of nuclear translocation of p-Smad2 comprises immunohistochemistry.

59. The method or TGFβ inhibitor for use according to any one of embodiments 54-58, wherein the determination of nuclear translocation of p-Smad2 comprises nuclear masking.

60. The TGFβ inhibitor or method of any one of embodiments 1-59, wherein the TGFβ inhibitor inhibits TGFβ1 signaling.

61. The TGFβ inhibitor or method of any one of embodiments 1-60, wherein the TGFβ inhibitor binds selectively to pro/latent-TGFβ.

62. The TGFβ inhibitor or method of embodiment 60, wherein the TGFβ inhibitor binds selectively to pro/latent-TGFβ1.

63. The TGFβ inhibitor or method of embodiment 61 or embodiment 62, wherein the TGFβ inhibitor does not bind to mature TGFβ1.

64. The TGFβ inhibitor or method of any one of embodiments 1-63, wherein the TGFβ inhibitor is a TGFβ1-selective inhibitor, wherein optionally the TGFβ1-selective inhibitor is a neutralizing antibody that binds mature TGFβ1 or an activation inhibitor that binds proTGFβ1.

65. The TGFβ inhibitor or method of embodiment 64, wherein the TGFβ1-selective inhibitor is:
a) a monoclonal antibody designated as Ab6 herein, a TGFβ1-selective variant thereof, or an antigen-binding fragment thereof or an antibody comprising the CDRs and/or variable domains from Ab6; or
b) an antibody or an antigen-binding fragment thereof that competes for binding and/or binds the same epitope as Ab6.

66. The TGFβ inhibitor or method of any one of embodiments 1-65, wherein the TGFβ inhibitor comprises an isolated antibody or antigen-binding fragment thereof comprising three heavy chain complementarity determining regions comprising amino acid sequences of SEQ ID NO: 1 (H-CDR1), SEQ ID NO: 2 (H-CDR2), and SEQ ID NO: 3 (H-CDR3) and three light chain complementarity determining regions comprising amino acid sequences of SEQ ID NO: 4 (L-CDR1), SEQ ID NO: 5 (L-CDR2), and SEQ ID NO: 6 (L-CDR3), as defined by the IMTG numbering system, wherein optionally the TGFβ inhibitor comprises an isolated antibody or antigen-binding fragment thereof comprising a heavy chain variable region comprising an amino acid sequence of SEQ ID NO: 7 and a light chain variable region comprising an amino acid sequence of SEQ ID NO: 8 or amino acid sequences 90% identical thereto.

67. The TGFβ inhibitor or method of any one of embodiments 1-66 wherein the TGFβ inhibitor is a TGFβ1/2 inhibitor, wherein optionally the inhibitor is NIS793/XOMA-089 or GC1008.

68. The TGFβ inhibitor or method of any one of embodiments 1-66, wherein the TGFβ inhibitor is a TGFβ1/3 inhibitor wherein optionally the inhibitor is M7824 (bintrafusp alpha) or AVID200.

69. The TGFβ inhibitor or method of any one of embodiments 1-68, wherein the TGFβ inhibitor is a low molecular weight antagonist of the TGFβ receptor kinase, wherein optionally the antagonist is an ALK5 inhibitor.

70. A method for treating cancer a human subject having a solid tumor, the method comprising administering to the subject a checkpoint inhibitor in an amount effective to reduce tumor growth, wherein the solid tumor has an inflamed phenotype characterized by containing greater than 5% intratumor CD8+ cells as assessed by an immunohistochemistry analysis capable of detecting individual tumor nest(s) within the tumor.

71. The method of embodiment 70, wherein the tumor is characterized by having greater than 50% tumor area comprising tumor nest(s) comprising greater than 5% CD8+ cells inside the tumor nest(s).

72. A TGFβ inhibitor for use in the treatment of cancer in a subject, wherein the treatment comprises administration of a TGFβ inhibitor, a cancer therapy, or a combination thereof, wherein optionally the cancer therapy is a checkpoint inhibitor therapy or genotoxic therapy, and wherein the treatment comprises determination of a level of MDSCs in a blood sample obtained from the subject, wherein optionally, the MDSC levels are measured with the use of an antibody that binds LRRC33, wherein further optionally, the antibody that binds LRRC33 is used in a FACS-based assay or ELISA-based assay.

73. The TGFβ inhibitor for use according to embodiment 72, wherein the treatment further comprises determination of CD8+ cells in a tumor biopsy sample obtained from the subject, optionally using an image analysis method that allows resolution of individual tumor nests in accordance with the disclosure herein.

74. The TGFβ inhibitor for use according to embodiment 72 or 73, wherein the treatment further comprises determination of circulatory TGFβ levels in a blood sample obtained from the subject, wherein the blood sample is collected and/or processed below room temperature, optionally in accordance with the disclosure herein, wherein optionally, the blood sample is processed to prepare a platelet-poor plasma (PPP) sample.

75. The TGFβ inhibitor for use according to any one of embodiments 72-74, wherein the TGFβ inhibitor is an isoform-selective inhibitor of TGFβ1, wherein optionally the isoform-selective inhibitor is a neutralizing antibody that selectively binds TGFβ1, or an antibody that binds a latent TGFβ1 complex; or wherein the TGFβ inhibitor is an inhibitor of TGFβ1 and TGFβ2, wherein optionally the inhibitor is NIS793/XOMA-089 or GC1008.

76. The TGFβ inhibitor for use according to any one of embodiments 48-50, wherein the TGFβ inhibitor is an inhibitor of TGFβ1 and TGFβ3, wherein optionally the inhibitor is M7824 (bintrafusp alpha) or AVID200.

77. The TGFβ inhibitor for use or method according to any one of embodiments 1-66 and 69-74, wherein the TGFβ inhibitor is a pan inhibitor that inhibits TGFβ1, TGFβ2 and TGFβ3, wherein optionally the pan inhibitor is GC1008 or derivative thereof, SAR439459, LY3022859, or an agent that blocks ligand-binding domain of a TGFβ receptor.

78. The TGFβ inhibitor for use or method according to any one of embodiments 1-66 and 69-74, wherein the TGFβ inhibitor is an activation inhibitor of TGFβ pathway that interferes with integrin-TGFβ interaction, wherein optionally the inhibitor is an agent that binds an integrin, or an agent that binds the RGD motif present in latent TGFβ1 and/or TGFβ3, wherein further optionally the agent is a low molecular weignt weight (small molecule) compound or an antibody or antigen-binding fragment thereof.

79. The TGFβ inhibitor for use or method according to any one of embodiments 1-66 and 69-74, wherein the TGFβ inhibitor is a low molecular weight (small molecule) antagonist of ALK5.

80. The TGFβ inhibitor for use or method according to any one of embodiments 1-66 and 69-74, wherein the TGFβ inhibitor is an RNA-based inhibitor of TGFβ1 expression.

81. The TGFβ inhibitor for use or method according to any one of embodiments 1-66 and 69-74, wherein the TGFβ inhibitor is a soluble ligand trap that comprises a ligand-binding fragment of a TGFβ receptor, capable of sequestering mature growth factor, wherein the mature growth factor is TGF@1, TGFβ2, TGFβ3, or combination thereof, wherein further optionally, the ligand trap is M7824 (bintrafusp alpha) or AVID200.

82. The TGFβ inhibitor for use or method according to any one of embodiments 70-81, wherein the checkpoint inhibitor is anti-PD-1 antibody, anti-PD-L1 antibody, anti-CTLA-4-antibody, anti-LAG3 antibody, or an antigen-binding fragment thereof.

83. The TGFβ inhibitor for use of embodiment 72, wherein the cancer therapy is a genotoxic therapy.

84. The method or TGFβ inhibitor for use of any one of embodiments 1-35, 52-69, wherein the TGFβ inhibitor is administered concurrently (e.g., simultaneously or sequentially) with a genotoxic therapy.

85. A method of treating solid cancer, comprising:
i) selecting a subject having a cancer with an elevated TGFβ level and/or TGFβ activity; and
ii) administering to the subject a therapeutically effective amount of a TGFβ inhibitor;
wherein the TGFβ inhibitor is administered concurrently (e.g., simultaneously or sequentially) with a genotoxic therapy.

86. A TGFβ inhibitor for use in treating solid cancer, comprising:
i) selecting a subject having a cancer with an elevated TGFβ level and/or signaling; and
ii) administering to the subject a therapeutically effective amount of a TGFβ inhibitor;
wherein the TGFβ inhibitor is administered concurrently (e.g., simultaneously or sequentially) with a genotoxic therapy.

87. The method or TGFβ inhibitor for use of embodiment 85 or embodiment 86, wherein the subject has intrinsic or acquired resistance to the genotoxic therapy.

88. The method or TGFβ inhibitor for use of embodiment 85 or embodiment 86, wherein TGFβ inhibitor produces synergistic effects with the genotoxic therapy.

89. The method or TGFβ inhibitor for use of any one of embodiments 83-88, wherein the genotoxic therapy is a chemotherapy or a radiation therapy.

90. The method or TGFβ inhibitor for use of any one of embodiments 83-89, wherein subject has ovarian cancer, breast cancer, bladder cancer, pancreatic cancer, e.g., pancreatic adenocarcinoma, prostate cancer, e.g., prostate adenocarcinoma, melanoma, e.g., skin cutaneous melanoma, lung cancer, e.g., lung squamous cell carcinoma and lung adenocarcinoma, liver cancer (e.g., liver hepatocellular carcinoma), uterine cancer, e.g., uterine corpus endometrial carcinoma, kidney cancer, e.g., renal clear cell carcinoma, head and neck cancer, e.g., head and neck squamous cell carcinoma, colon cancer, e.g., colon adenocarcinoma, esophageal carcinoma, or tenosynovial giant cell tumor (TGCT).

91. The method or TGFβ inhibitor for use of any one of embodiments 83-90, wherein the TGFβ inhibitor comprises an isolated antibody or antigen-binding fragment thereof comprising three heavy chain complementarity determining regions comprising amino acid sequences of SEQ ID NO: 1 (H-CDR1), SEQ ID NO: 2 (H-CDR2), and SEQ ID NO: 3 (H-CDR3) and three light chain complementarity determining regions comprising amino acid sequences of SEQ ID NO: 4 (L-CDR1), SEQ ID NO: 5 (L-CDR2), and SEQ ID NO: 6 (L-CDR3), as defined by the IMTG numbering system, wherein optionally the TGFβ inhibitor comprises an isolated antibody or antigen-binding fragment thereof comprising a heavy chain variable region comprising an amino acid sequence of SEQ ID NO: 7 and a light chain variable region comprising an amino acid sequence of SEQ ID NO: 8 or amino acid sequences 90% identical thereto.

92. A method of distinguishing MDSC populations from a biological sample obtained from a patient, wherein gMDSC populations are identified by cell surface markers of CD11b+, HLA-DR−, CD14−, CD15+, CD33+/low, and CD66b+; and wherein mMDSC populations are identified by cell surface markers of CD11 b+, HLA-DR−/low, CD14+, CD15−, CD33+/high, and CD66b−.

93. The method of embodiment 92, wherein the identification comprises binary intensity selection.

94. The method of embodiment 93, wherein the binary intensity selection comprises binning signal intensities according to one or more of the cutoff points shown in Table 38B.

95. The method of any one of embodiments 92-94, wherein the identification comprises sequential application of signal filtering.

96. The method of any one of embodiments 92-95, wherein the identification comprises signal binning according to HLD-DR signal intensity, wherein a signal intensity of <0.105 is binned as negative signal, a signal intensity of 0.105-0.125 is binned as low signal, a signal intensity of 0.125-0.155 is binned as medium signal, and a signal intensity of greater than 0.155 is binned as high signal.

97. The method of any one of embodiments 92-96, wherein the identification comprises signal binning according to CD33 signal intensity, wherein a signal intensity of <0.17 is binned as negative signal, a signal intensity of 0.17-0.18 is binned as low signal, a signal intensity of 0.18-0.19 is binned as medium signal, and a signal intensity of greater than 0.19 is binned as high signal.

### EXAMPLES

It will be readily apparent to those skilled in the art that other suitable modifications and adaptations of the composition and methods described herein may be made using suitable equivalents without departing from the scope of the disclosure or the embodiments disclosed herein. This disclosure is further illustrated by the following examples which should not be construed as limiting.

We previously observed that tumor-associated MDSCs express cell-surface LRRC33 in tumor-bearing mice. Here, we investigated whether circulating MDSCs might also express cell-surface LRRC33. Previously, we and others found that presence of LRRC33 mRNA transcripts does not always correlate with protein expression of LRRC33, particularly cell-surface LRRC33. Therefore, FACS experiments were carried out to assess whether circulating MDSCs in tumor-bearing mice express cell-surface LRRC33. A monoclonal antibody that binds cell-surface LRRC33, and an IgG control, were used to isolate/enrich MDSCs, including the G-MDSC and M-MDSC pools from blood samples collected from MBT-2 mice. Data show robust and substantially uniform expression of LRRC33 in circulating MDSCs. In fact, nearly all MDSCs, including G-MDSCs and M-MDSCs, in circulation appear to express cell-surface LRRC33 at high levels. Taken together, these data show that LRRC33 is expressed on cell surface by circulating MDSCs, as well as in tumor (i.e., tumor-associated MDSCs), that circulating monocytes do not express LRRC33, and that LRRC33 is identified as a novel blood-based marker for MDSCs. This raises the possibility that LRRC33 may serve as a surrogate blood-based biomarker for immunosuppression, particularly in cancer.

### Example 1: Detection of Circulating MDSCs

The effects of Ab6 treatment on circulating immune cell subsets in vivo were determined using an MBT-2 mouse model. Tumor-bearing mice were dosed with 10 mg/kg of Ab6 alone on days 1 and 8 or in combination with an anti-PD-1 antibody dosed on days 1, 4, and 8 at 10 mg/kg.

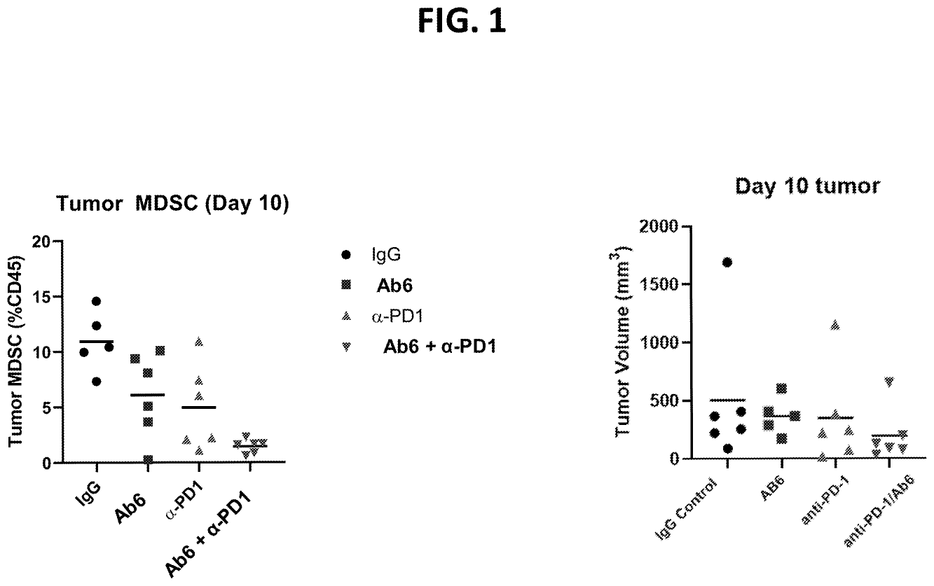

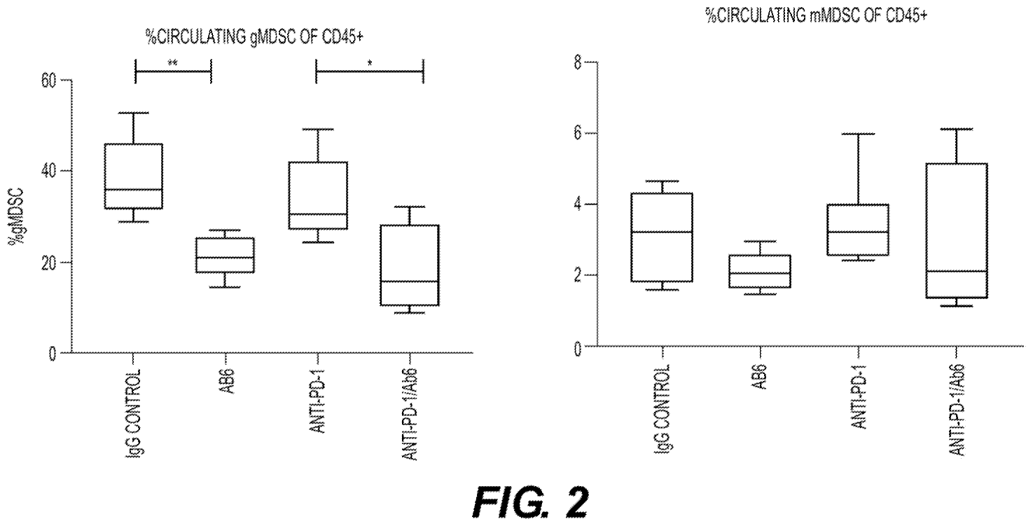

Whole blood was collected on day 10 and processed for flow cytometry analysis. Levels of circulating G-MDSCs and M-MDSCs were determined based on the expression of surface protein markers for G-MDSCs (CD45+CD11 b+Ly6G+Ly6C^{low}) and M-MDSCs (CD45+CD11 b+Ly6G-Ly6C^{high}). Values were expressed as percentages of total CD45+ cells detected in the blood. Circulating G-MDSC levels were decreased in groups treated with both Ab6 alone and combination treatments (i.e. Ab6 in combination with anti-PD-1), whereas circulating M-MDSC levels in Ab6-treated groups did not differ as compared to groups treated with IgG control or anti-PD-1 treatment alone. Tumor and circulating MDSC levels as assessed at day 10 following treatment initiation are shown in FIG. 1 and FIG. 2.

Flow cytometry analysis of T-cells was also performed at day 10 following treatment initiation from whole blood. Circulating T-cell levels were determined based on the expression of T cell surface protein markers. CD8+ and CD3+ T cell levels and values were normalized to total circulating CD45+ cells detected in whole blood. Groups treated with Ab6 alone exhibited a slight increase in both CD8+ and CD3+ circulating T-cell levels compared IgG control. Ab6 and anti-PD-1 combination treatment did not lead to significant changes in either CD8+ or CD3+ circulating T cell levels.

A second in vivo study was carried out to further evaluate the effects of Ab6 treatment on circulating and intratumoral MDSC populations. MBT-2 mice were treated with IgG control, Ab6 alone (10 mg/kg), an anti-PD-1 antibody (10 mg/kg), or a combination of Ab6 (1 mg/kg, 3 mg/kg, or 10 mg/kg) with an anti-PD-1 antibody (10 mg/kg). Treatments were administered on day 1 and 8. Whole blood was collected on day 17 prior to administering the first dose of treatment, and days 3, 6, and 10. Tumor volume was monitored throughout the study, and intratumoral MDSC analysis was carried out at day 10.

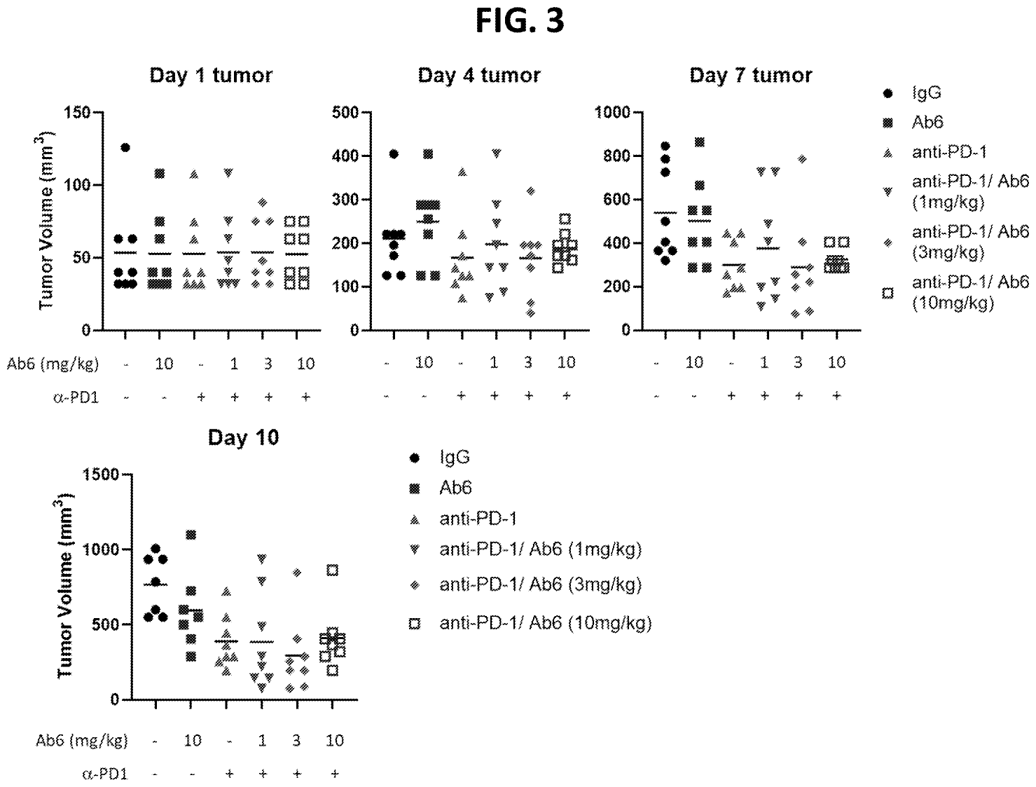

Measurement of tumor volume on days 1, 4, 7, and 10 showed a statistically significant treatment response in animals treated with anti-PD-1 antibody alone and in all animals treated with the combination of anti-PD-1 antibody and Ab6, but not in animals treated with Ab6 alone before day 10 (FIG. 3). The lack of treatment response observed in animals treated with Ab6 alone before day 10 was unlikely the result of incorrect dosing, as pharmacokinetic results confirmed that all animals were administered the correct Ab6 dosage. These results indicate that, in the case of MBT-2 tumors, concurrent inhibition of PD-1 and TGFβ1 pathways can reduce (e.g., delay or regress) tumor growth to a greater extent than inhibition of the PD-1 or TGFβ1 pathway alone.

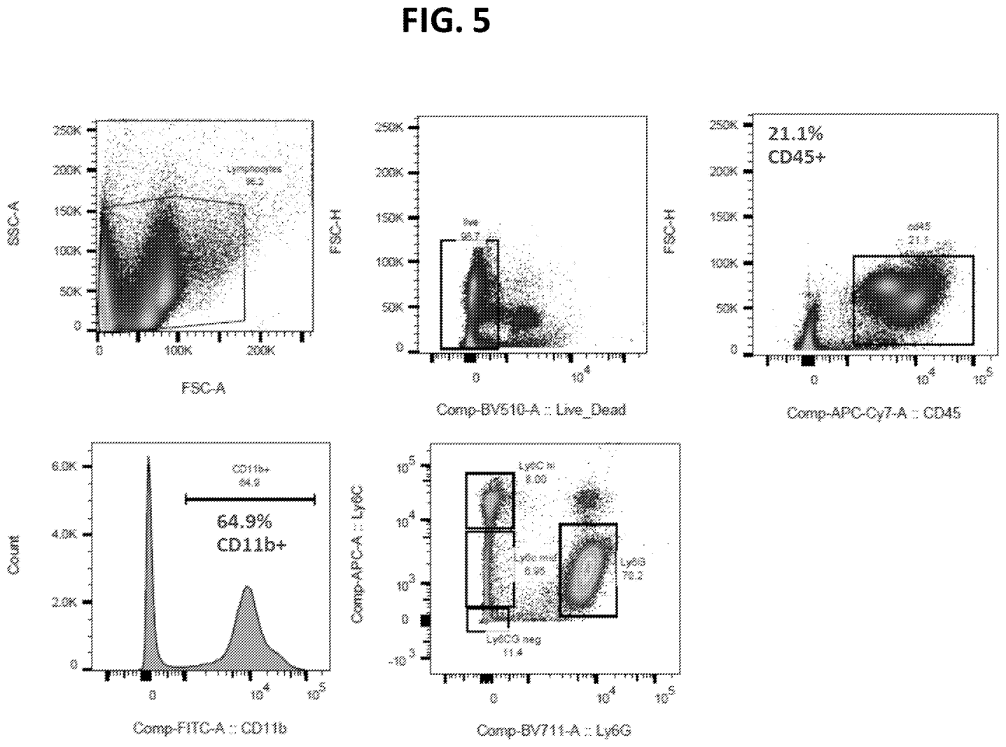

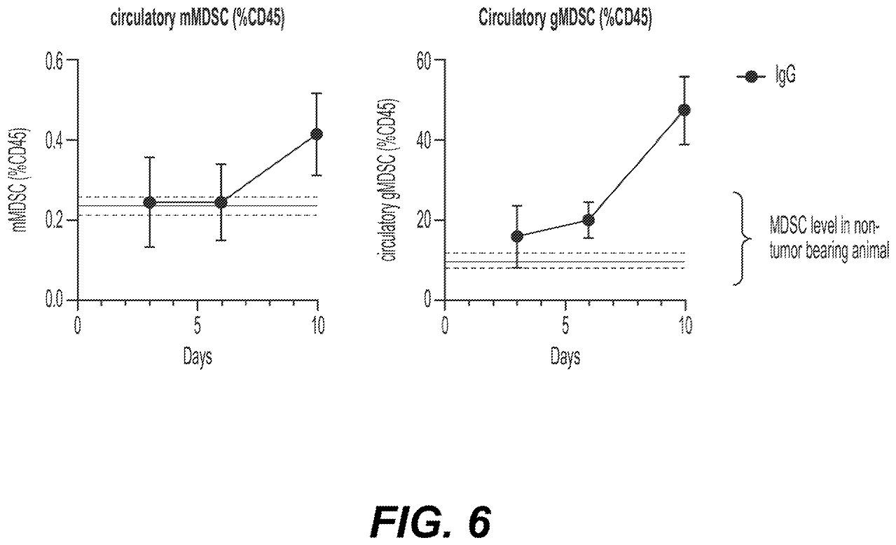

Levels of circulating immune cell populations were determined from whole blood samples via FACS analysis. Total CD11 b+ myeloid cells were identified from whole blood, from which M-MDSC populations were then identified by the expression of cell surface markers Ly6C (Ly6C^{high}), and G-MDSC populations were identified by the expression of cell surface marker Ly6G (Ly6G+). Baseline circulating MDSC levels were determined from whole blood samples collected from non-tumor bearing mice and consisted of 17.8% myeloid cells, which comprised 30% G-MDSCs and 29.1% M-MDSCs (percentages of total myeloid population) (FIG. 4). Levels of circulating immune cells were assessed on day 10 from tumor-bearing mice. Compared to baseline levels of non-tumor bearing mice, tumor-bearing mice exhibited markedly increased levels of total myeloid population and circulating G-MDSCs, but not M-MDSC cells (FIG. 6). Blood samples from tumor-bearing mice were found to consist of 64.9% myeloid cells, which comprised 70% G-MDSCs and 6.95 M-MDSCs. Furthermore, G-MDSCs were also found to make up 45.4% of the total CD45+ immune cell population in the blood of tumor-bearing mice, as compared to 5.45% of total CD45+ cells in the blood of non-tumor bearing mice (FIG. 5).

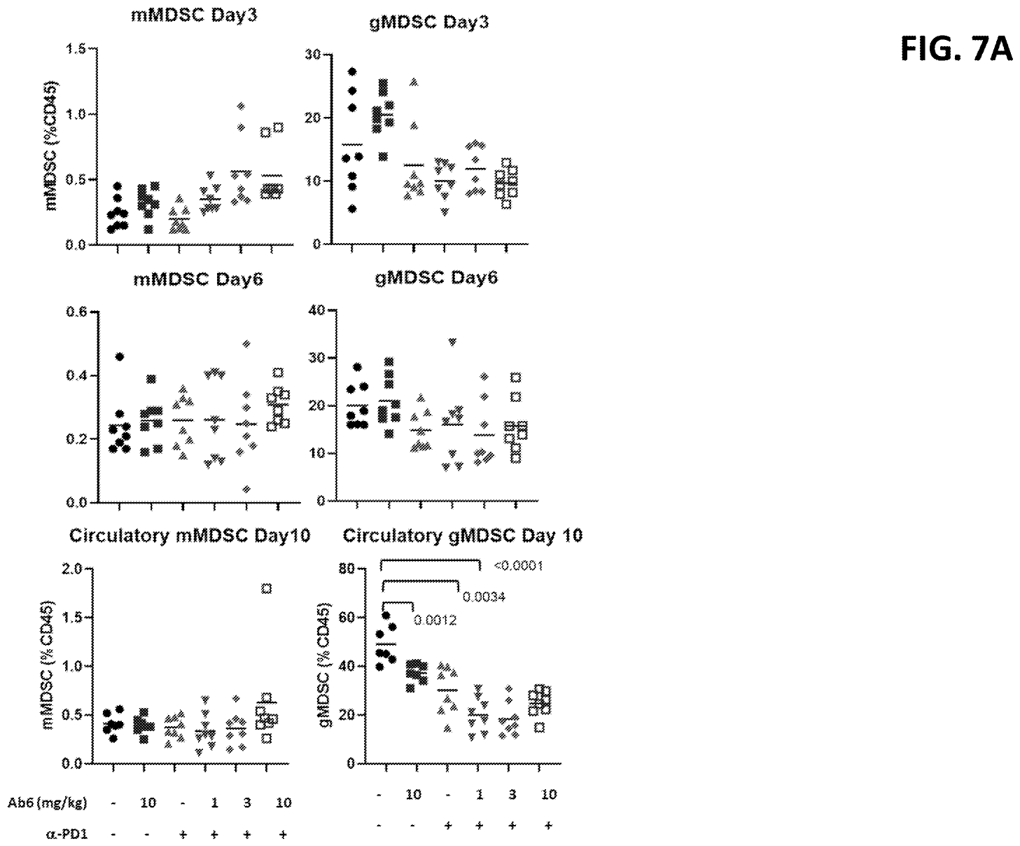

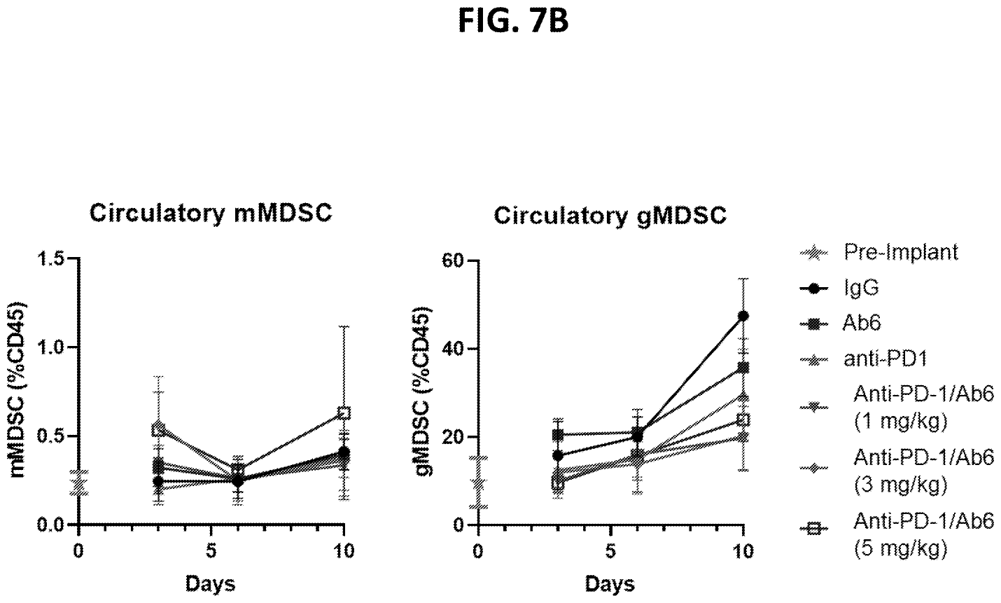

Circulating MDSC populations were evaluated in tumor-bearing animals throughout treatment. As shown in FIG. 6, levels of M-MDSCs remained low throughout treatment, whereas levels of G-MDSCs exhibited a decreasing trend, with statistically significant decreases in G-MDSC levels detected in all groups by day 10. A decrease in circulating G-MDSC levels in animals treated with Ab6 alone was not observed until day 10 (FIG. 7A and FIG. 7B). This suggests that Ab6 treatment alone may be sufficient to reduce circulating MDSC levels albeit a delayed rate as compared to a combination of Ab6 and anti-PD-1 treatment.

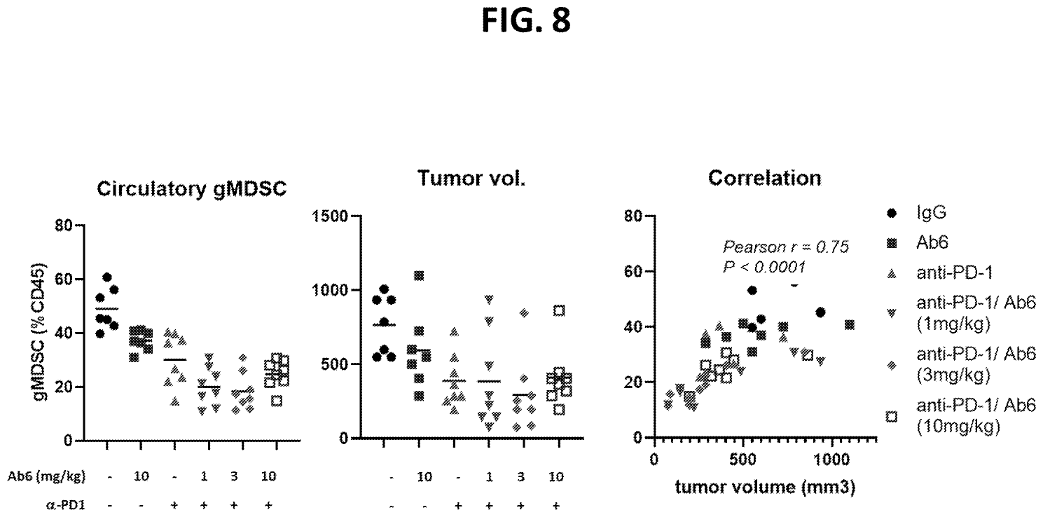

The association of circulating G-MDSC levels to tumor volume was also assessed at day 10. FIG. 8 shows a linear correlation between circulating G-MDSC levels and tumor volume in all groups.

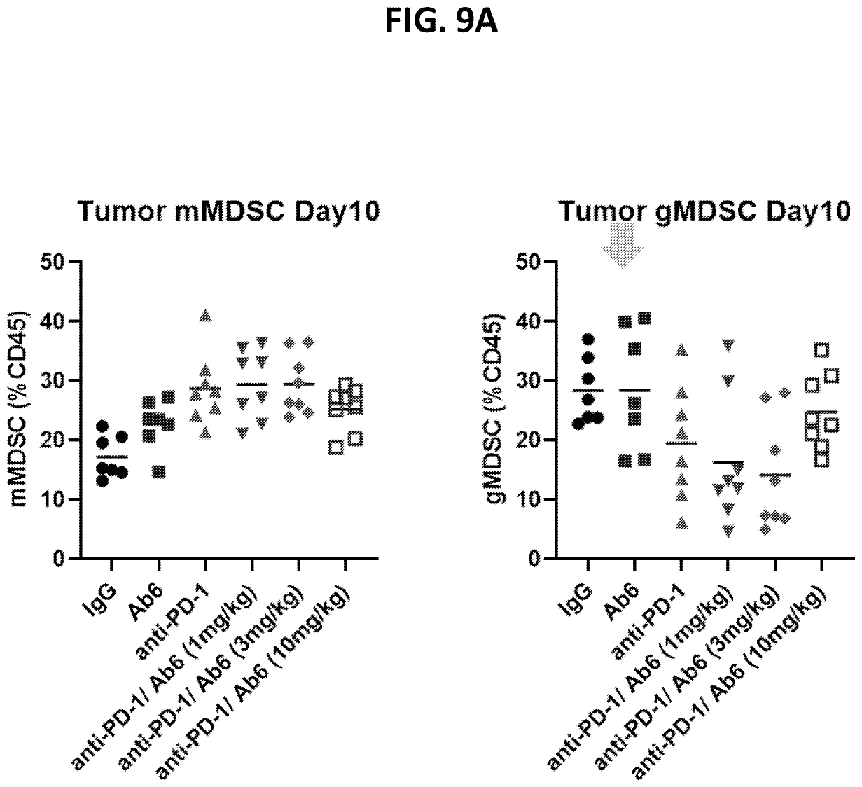

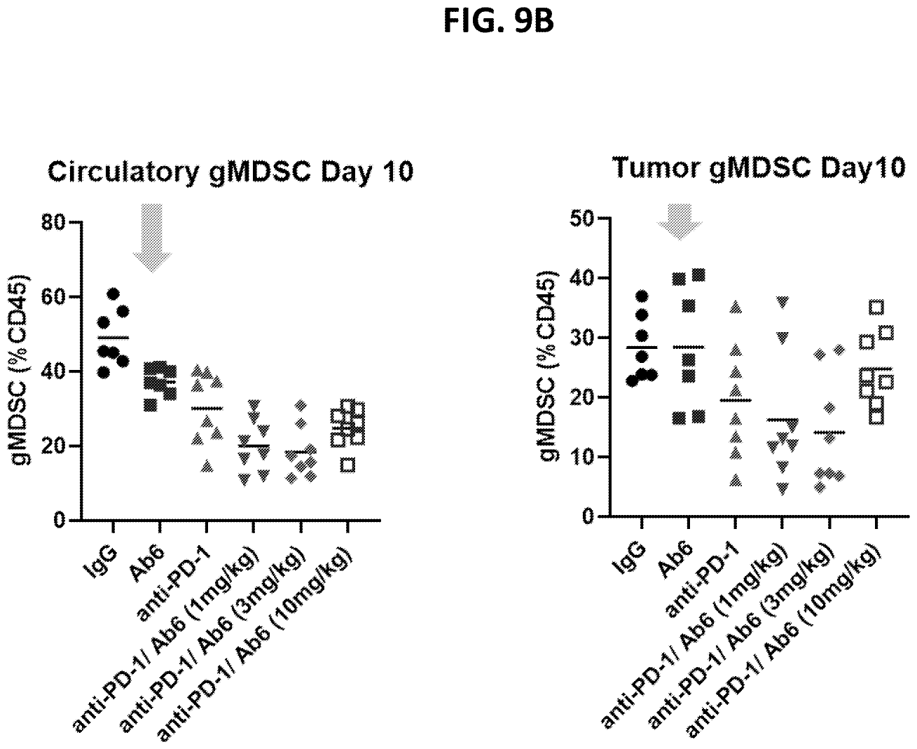

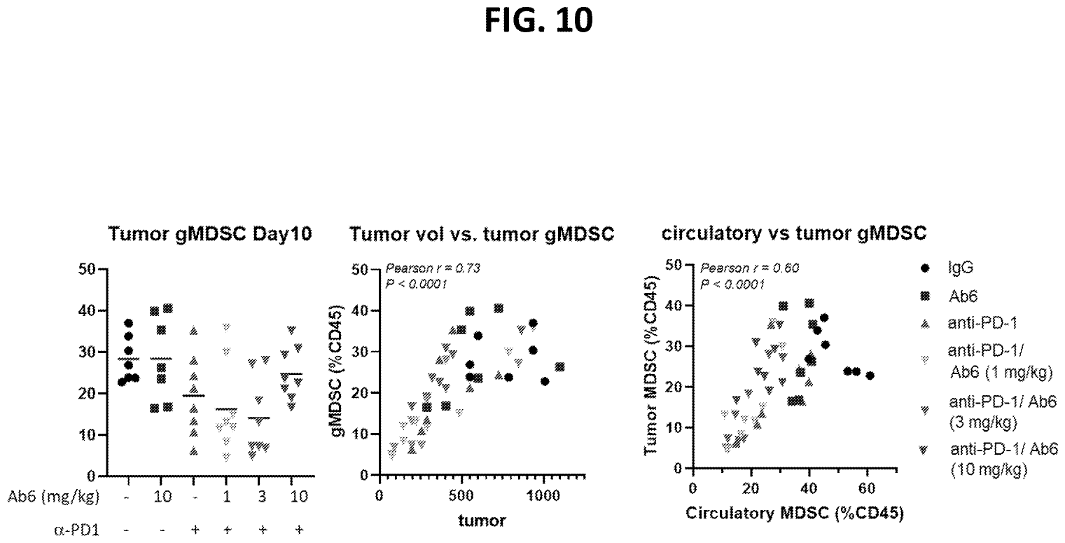

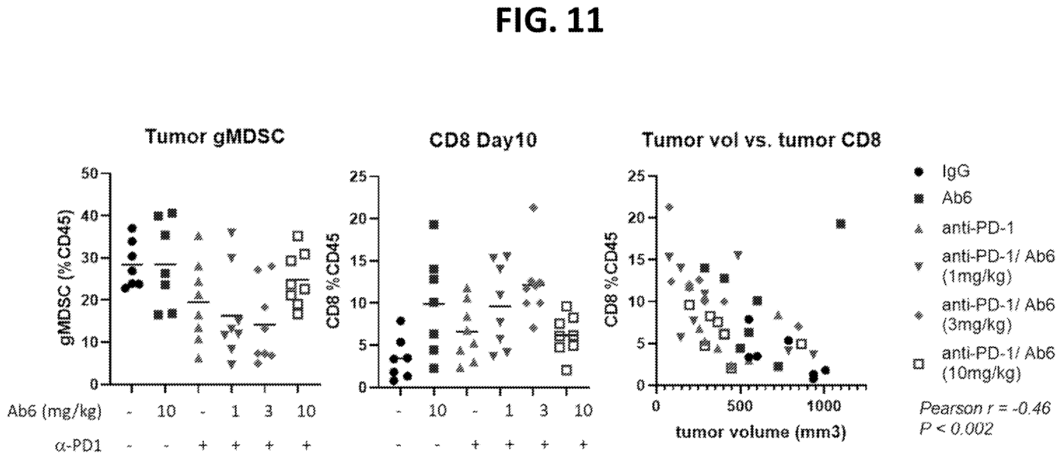

Levels of circulating and intratumorial MDSCs were compared to tumor volume measurements at day 10. Intratumoral M-MDSC levels in treated animals were similar across all treatment groups and did not decrease as compared to control animals. In contrast, intratumoral G-MDSC levels in animals treated with anti-PD-1 antibody alone or combination of anti-PD-1 antibody and Ab6 were reduced as compared to control animals (FIG. 9A). While Ab6 treatment alone resulted in a decrease in circulating G-MDSC levels, intratumoral MDSC levels were not affected by Ab6 treatment alone (FIG. 9B). A correlation of relative MDSC levels in tumor and in circulation is shown in FIG. 10. Additionally, reduced intratumoral G-MDSC levels at day 10 were found to correlate with elevated tumor CD8+ cells across all treatment groups (FIG. 11), suggesting a decrease in overall tumor immune suppression.

### Example 2: In Vivo Assessment of LRRC33 Expression on Circulating MDSC in MBT2 Syngeneic Bladder Cancer Mouse Model

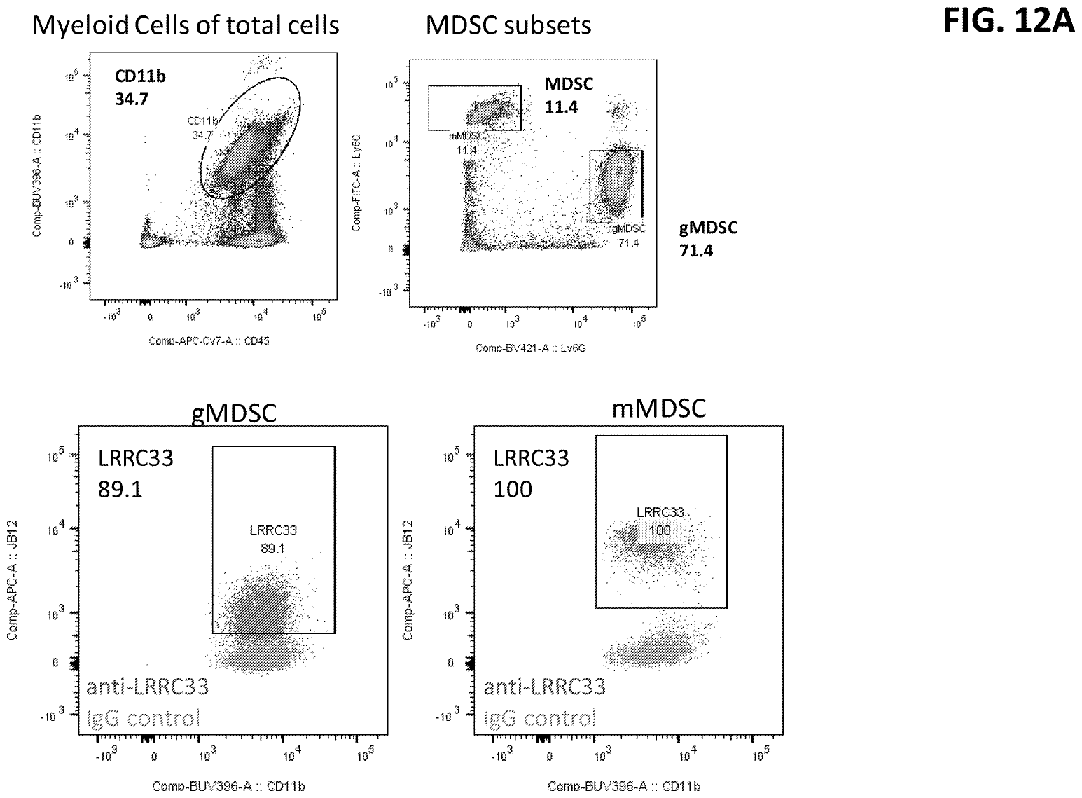

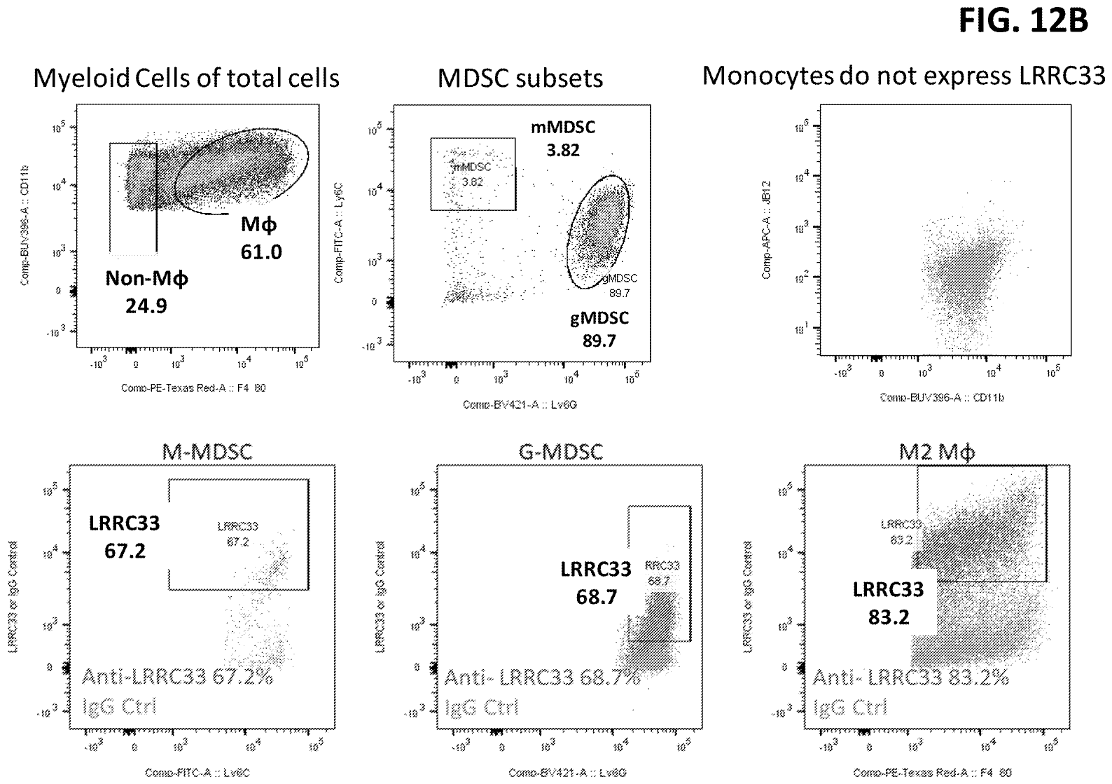

LRRC33 expression levels on circulating MDSCs were determined using FACS analysis using whole blood samples collected from mice bearing MBT2 tumors. M-MDSC and G-MDSC populations were identified on the basis of Ly6C and Ly6G expression, respectively, and then further characterized using a monoclonal antibody that binds cell-surface LRRC33, and an IgG control. Nearly all circulating MDSCs, including both G-MDSCs and M-MDSCs, appeared to express LRRC33. As shown in FIG. 12A, LRRC33 expression was detected on 89.1% of G-MDSCs and 100% of the M-MDSCs from whole blood of the tumor bearing mice. Consistent with previous observations, LRRC33 was also detected on intratumoral MDSCs, including G-MDSCs and M-MDSCs, albeit at varying levels (FIG. 12B). In contrast, LRRC33 expression was not detected on monocytes.

### Example 3: Ab6 Treatment Modulates Circulatory TGFβ1 Levels

Circulating TGFβ1 levels were assessed before and after treatment with Ab6 and/or an anti-PD-1 antibody in an MBT-2 mouse model. Tumor-bearing mice were dosed on days 1 and 8 with Ab6 alone at 10 mg/kg, a PD-1 antibody alone at 10 mg/kg, or 1 mg/kg, 3 mg/kg, or 10 mg/kg of Ab6 in combination with 10 mg/kg of the anti-PD1 antibody. Control animals were dosed with IgG control.

Blood samples were collected 14 before tumor implantation (day −14), 1 day before treatment, and on days 3, 6, and 10 following treatment. Samples were processed, including an acid treatment step, and circulatory TGFβ1 levels (pg/mL) were determined using an enzyme-linked immunosorbent assay (ELISA, e.g., R&D Systems Quantikine® assay). The acid treatment step liberates TGFβ1 from its latent complex and, without being bound by theory, it is believed that most of the TGFβ1 in circulation is present in the latent complex. Blood samples were also analyzed for platelet activation as a quality control measure. Samples with significant platelet activation were not further analyzed for circulating TGFβ levels because such samples were likely to contain TGFβ released from the activated platelets, thus likely rendering the TGFβ readout inaccurate. Platelet factor 4 (PF4) concentration was used as a surrogate marker for identifying samples containing significant platelet activation. Samples with high PF4 levels (greater than 500 ng/ml) were identified as having significant platelet activation and thus eliminated from further analysis for TGFβ concentration. Pharmacodynamic readouts such as assessment of tumor size, target engagement, and immune infiltration are carried out using tumor samples.

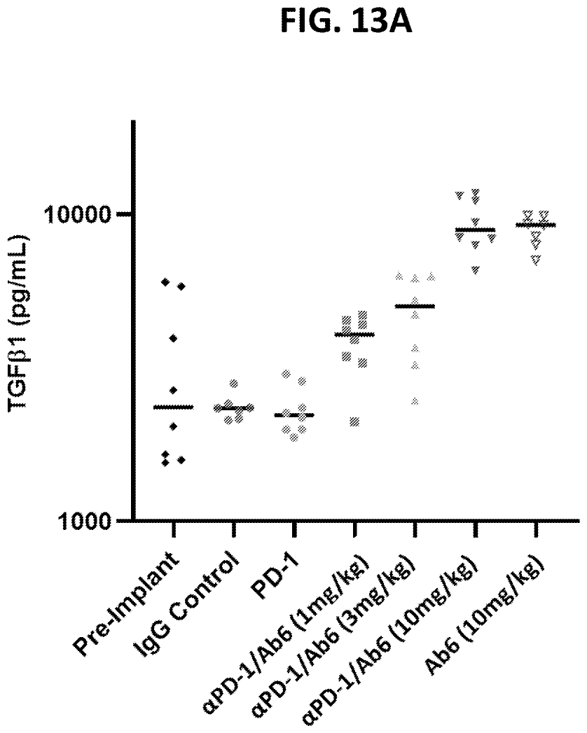

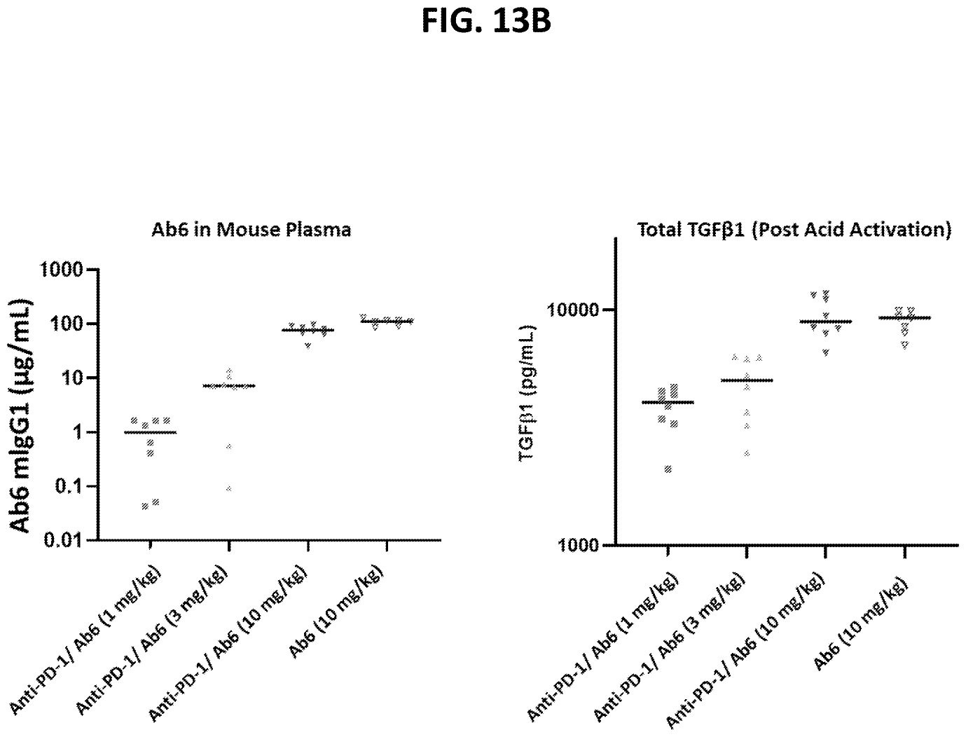

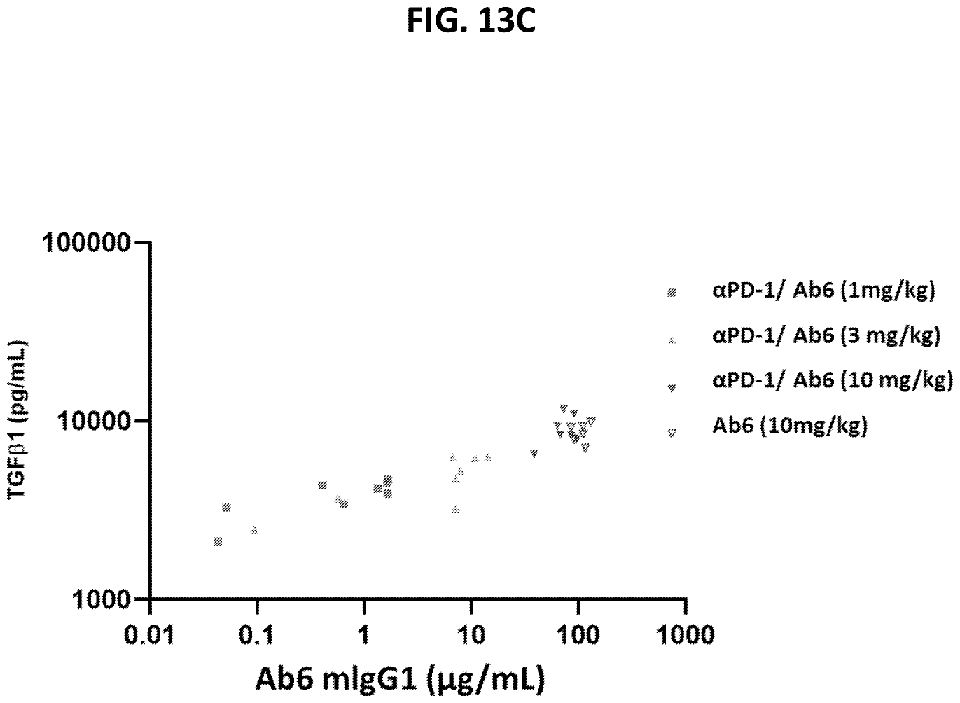

Results showed an increasing trend of circulating TGFβ1 levels in animals treated with Ab6 (alone or in combination with the anti-PD-1 antibody) as compared to circulating TGFβ1 levels pre-implantation and in IgG control-treated animals. In contrast, animals treated with the anti-PD1 antibody alone did not exhibit increased circulating TGFβ1 levels compared to controls (FIG. 13A). A statistically significant correlation was found between circulating TGFβ1 levels and plasma levels of Ab6 for each treatment group (R²=0.714) (FIG. 13B and FIG. 13C).

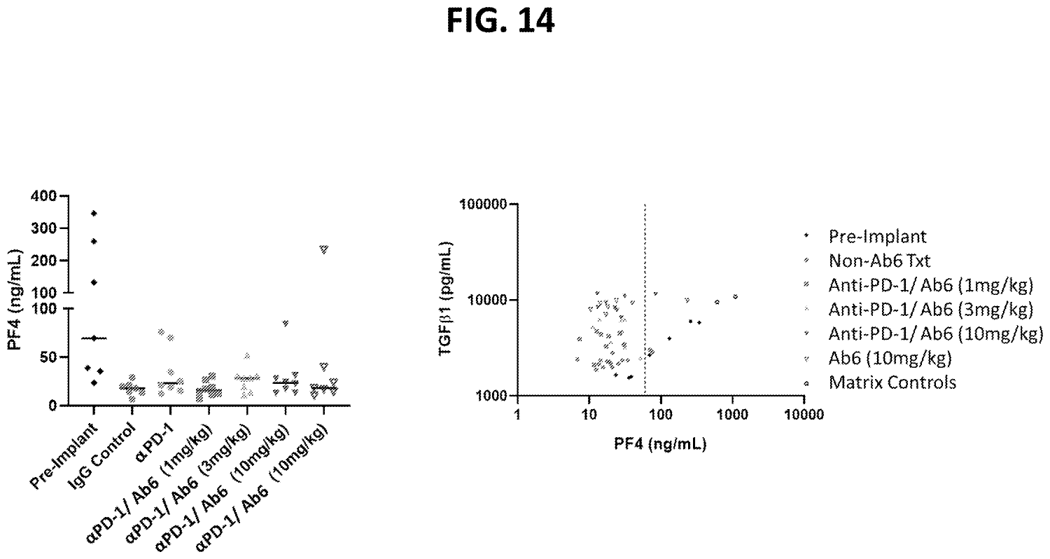

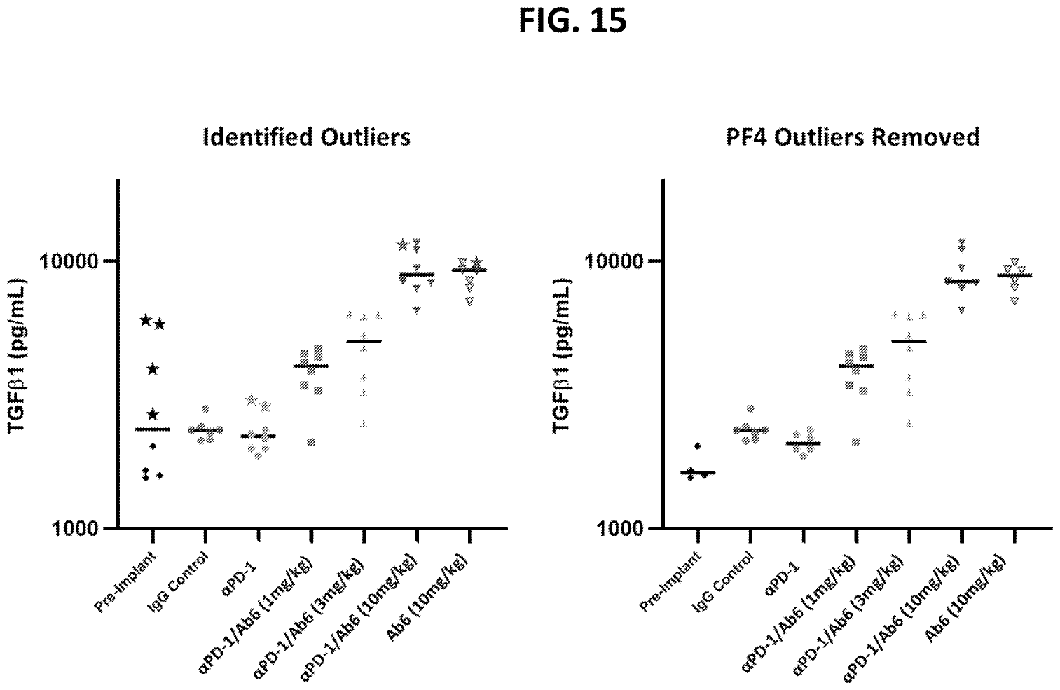

In order to avoid measuring additional circulatory TGFβ1 released as an artifact of sample collection and processing, plasma platelet factor 4 (PF4) levels were determined in each sample by ELISA and resulting PF4 levels were used to normalize circulatory TGFβ1 release. PF4 levels may be used as an indicator of platelet activation induced during sample collection that may contribute to TGFβ1 release. PF4 levels (ng/mL) were found to be low across drug-treated and IgG control samples. In comparison, pre-implant samples exhibited higher PF4 levels (FIG. 14, left panel). Sample outliers were identified using interquartile range, with an upper bound PF4 level of 60.45 ng/mL and a lower bound of 42.41 ng/mL (FIG. 14, right panel). Results corrected for PF4 outliers showed a statistically significant, dose-dependent increase in circulating TGFβ1 levels following Ab6 treatment (alone or in combination with the anti-PD-1 antibody). Furthermore, outlier-correct results also revealed elevated circulating TGFβ1 levels in tumor-bearing animals as compared to non-tumor-bearing controls (pre-implantation) (FIG. 15). As shown in FIG. 15, outlier-corrected total circulatory TGFβ1 levels were about 2000 μg/mL pre-implantation, about 3000 μg/mL in mice treated with IgG control, about 2500 μg/mL in mice treated with anti-PD-1 alone, about 9000 μg/mL in mice treated with 10 mg/kg of Ab6 alone, and above 6000 μg/mL, 7200 μg/mL, and 9000 μg/mL in mice treated with combination therapy comprising 1 mg/kg, 3 mg/kg, and 10 mg/kg of Ab6, respectively.

Without wishing to be bound by theory, PF4 levels may be useful for identifying and eliminating samples contaminated by platelet activation during sample collection and processing. In some embodiments, a sample with a PF4 concentration of greater than 500 ng/mg may indicate that the sample contains platelet activation. In some embodiments, the sample with a PF4 concentration of greater than 500 ng/mg may be treated as an outlier sample.

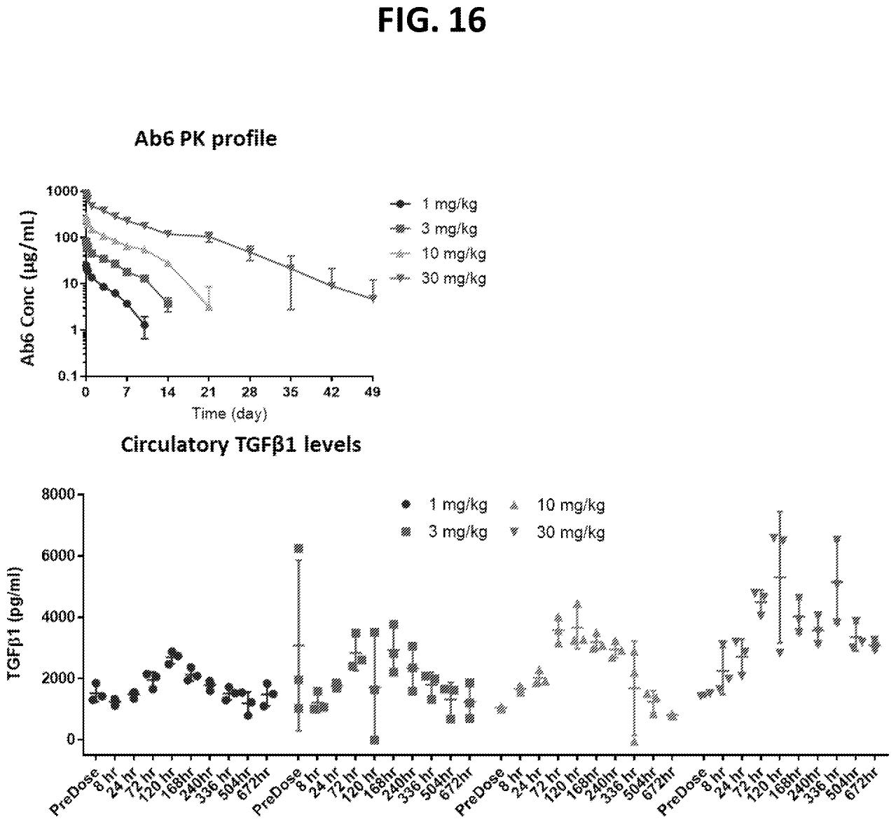

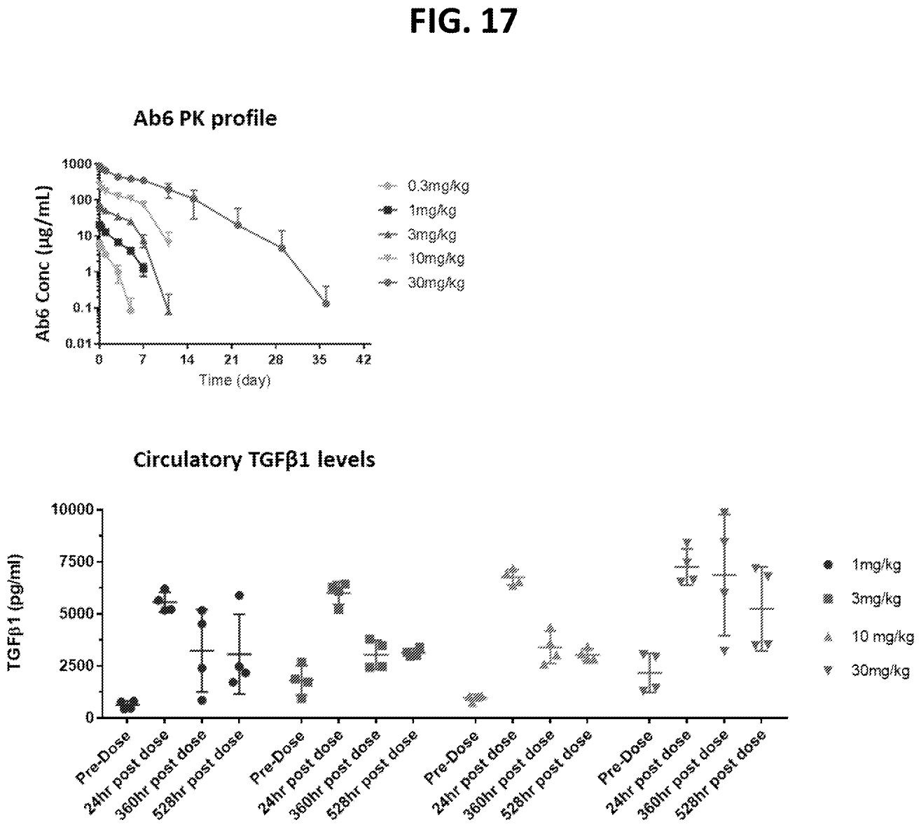

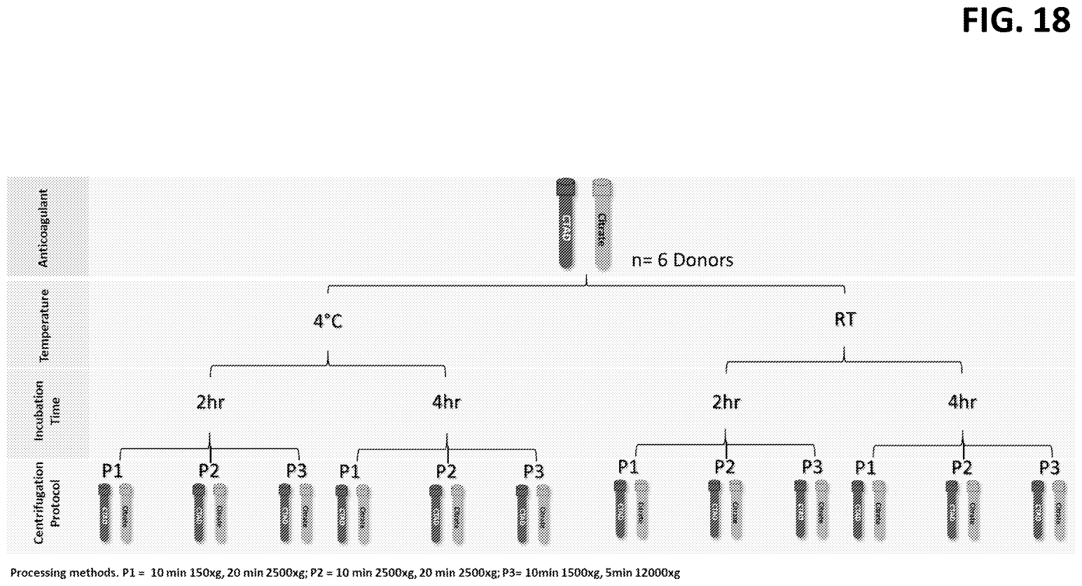

Similar trends of dose-dependent increase in circulatory TGFβ1 levels were also observed in non-human primates and rats treated with a single dose of Ab6. In non-human primates treated with 1 mg/kg, 3 mg/kg, 10 mg/kg, or 30 mg/kg of Ab6, the extent and duration of increases in circulatory TGFβ1 levels were dose-dependent. Circulatory TGFβ1 levels were measured around 72-240 hrs following Ab6 administration, and peak circulatory TGFβ1 levels were between about 2000 μg/ml to about 4000 μg/ml (FIG. 16). Similarly, rats that were treated with 0.3 mg/kg, 1 mg/kg, 3 mg/kg, 10 mg/kg, or 30 mg/kg of Ab6 also exhibited dose-dependent increase in circulatory TGFβ1 levels. Circulatory TGFβ1 levels were detected around 24-360 hrs following Ab6 administration, with peak circulatory TGFβ1 levels detected between about 5000 μg/ml to about 7500 μg/ml. Duration of circulatory TGFβ1 elevation for each treatment group appeared to be dose-dependent (FIG. 17).

### Example 4: Sample Collection and Processing Method for Assessment of Circulating Latent TGFβ1 in Human Blood Samples

A pilot study was conducted to identify improved collection and processing conditions for assessing circulation latent TGFβ1 in human plasma samples. Whole blood samples were collected from 6 healthy donors and processed under various conditions to generate platelet poor plasma (PPP), including variations in temperature, types of collection tubes, time prior to processing, and centrifugation conditions (FIG. 18, Table 15). PPP processing was assessed using tubes coated with anticoagulant citrate-theophylline-adenosine-dipyridamole (CTAD) or sodium citrate (TSC). Samples were processed after 2 hours or 4 hours of incubation following sample collection. Samples were centrifuged according to one of three centrifugation protocols: a first step of 150×g for 10 minutes followed by a second step of 2500×g for 20 minutes (P1), a first step of 2500×g for 10 minutes followed by a second step of 2500×g for 20 minutes (P2), and a first step of 1500×g for 10 minutes followed by a second step of 12000×g for 5 minutes (P3). Acceleration and deceleration of the centrifugation steps were set at 20% of centrifuge maximum. Following the first centrifugation step, the supernatant portion of each tube was transferred to a separate tube, removing approximately 70% of the volume without disturbing buffy coat and red cell layer, and the supernatant is further processed the second centrifugation step. The resulting supernatant was used to measure circulating TGFβ1 levels. Sample incubation and processing were carried out under 40C or room temperature (RT).

TABLE 15
Sample processing conditions

| Assay Component | Parameters | |
|---|---|---|
| Anticoagulant | CTAD | |
| | TSC | |
| Temperature | 4° | C. |
| | RT | |
| Incubation time | 2 | hours |
| | 4 | hours |

| | |
|---|---|
| Centrifugation | P1: 10 min at 150 × g, 20 min at 2500 × g |
| protocol | P2: 10 min at 2500 × g, 20 min at 2500 × g |
| | P3: 10 min at 1500 × g, 5 min at 12000 × g |

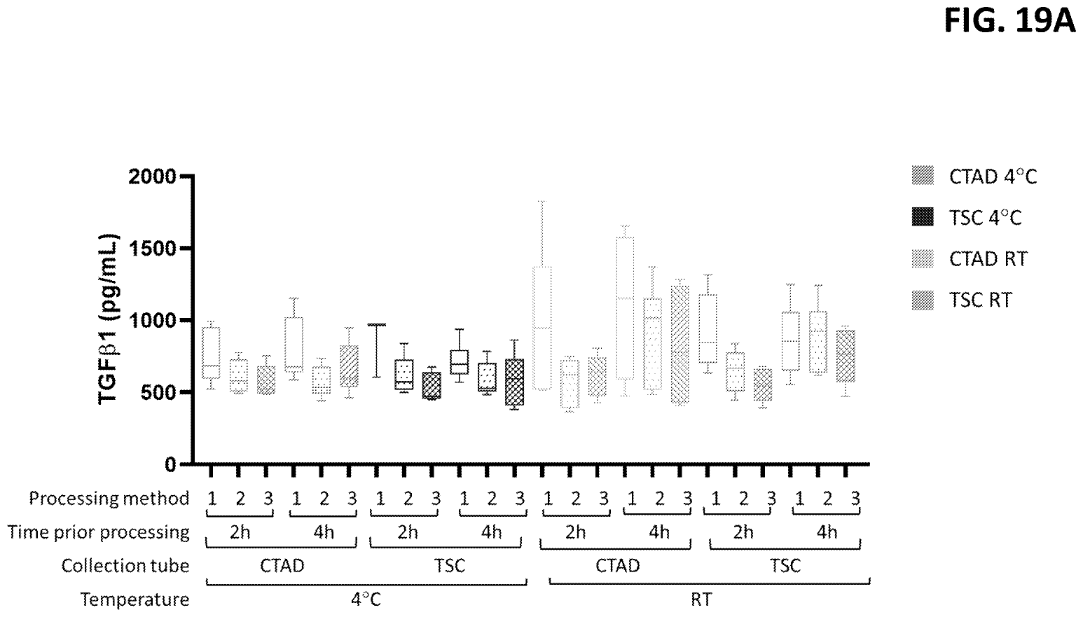

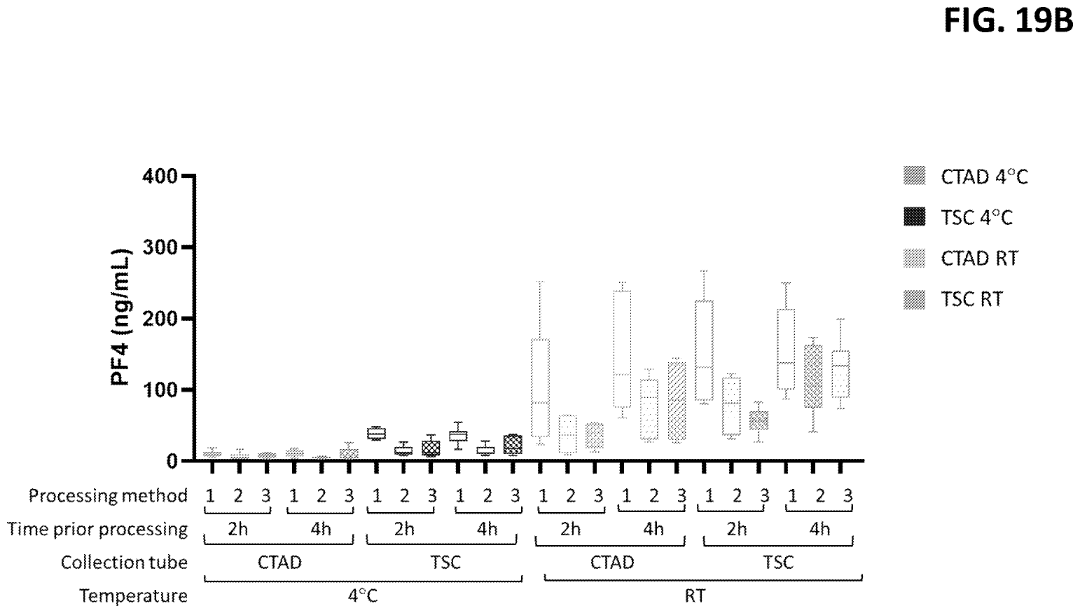

Circulatory TGFβ1 was evaluated by ELISA after acidification of PPP, using the using R&D System Quantikine assay. The average concentration of TGFβ1 ranged from 500 μg/ml to 1500 μg/ml (FIG. 19A). Samples processed at 4° C. showed significantly lower level of TGFβ1 as compared to samples processed at room temperature. Platelet Factor 4 (PF4) levels were evaluated as control for platelet-induced TGFβ1 activation. Significantly higher level of TGFβ1 and PF4 were detected in samples processed at room temperature as compared to samples processed at 4° C. (FIG. 19B). Lowest level of PF4 was observed in samples collected in CTAD tubes and processed under centrifugation condition P2 (FIG. 19B). Correlation of PF4 and TGFβ1 was observed in samples processed at room temperature (FIG. 20). Exemplary analyses of outlier removal based on high PF4 levels are shown in FIGS. 21A-21C. Regression analysis used in FIG. 21B is described in Motulsky and Brown. BMC Bioinformatics. 2006 Mar. 9; 7:123.

### Example 5: In Vivo Assessment of Circulating Latent TGFβ1 in Human Plasma Samples

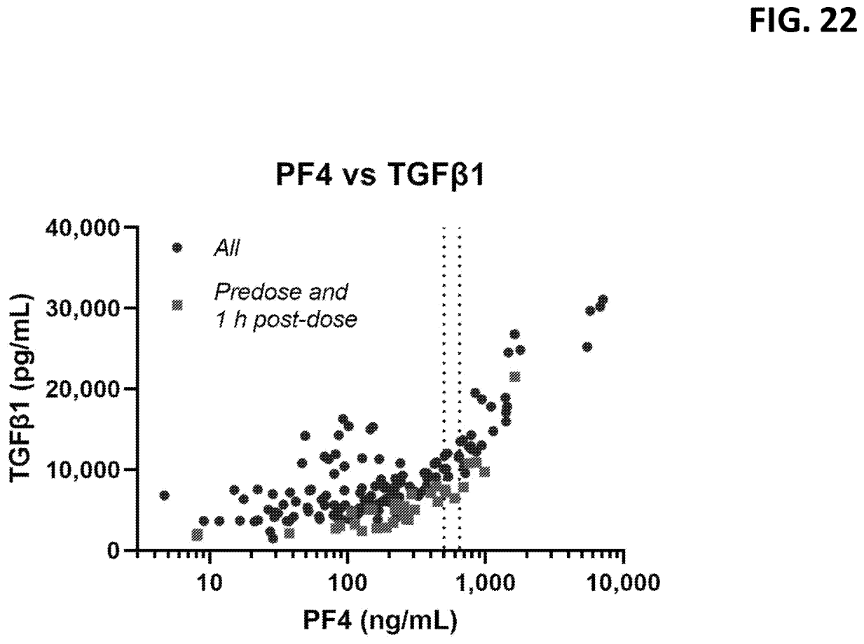

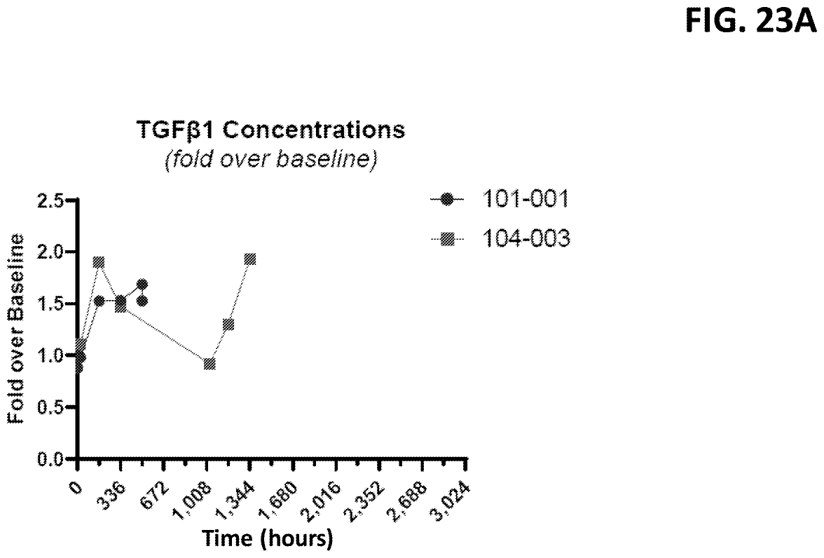

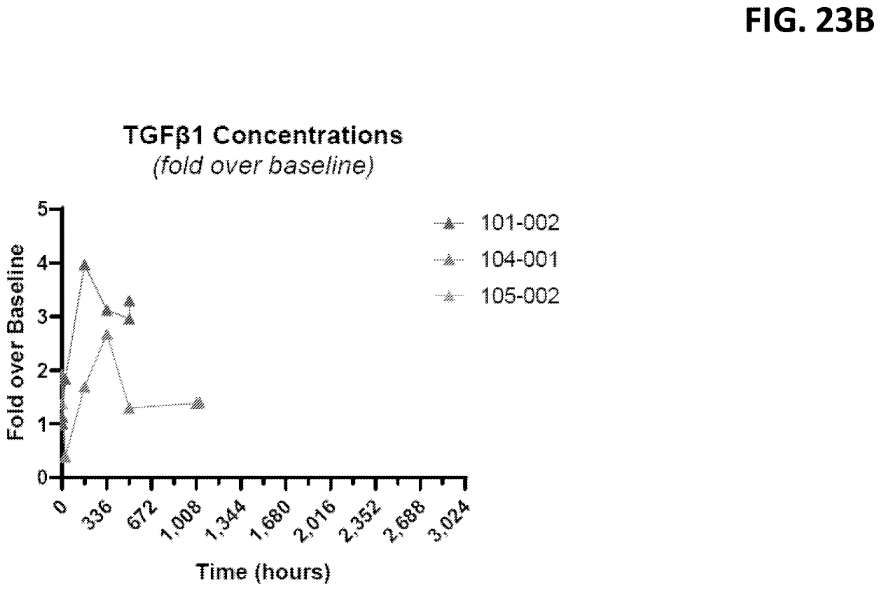

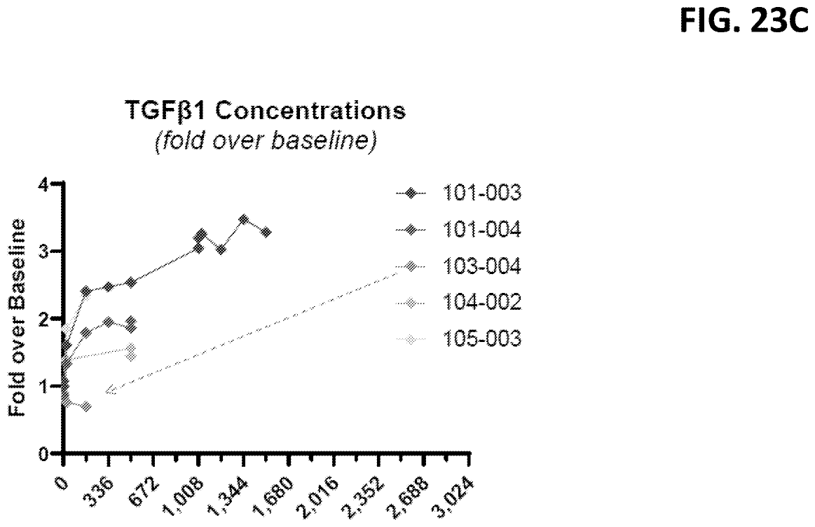

Circulating latent TGFβ levels were assessed in human platelet-poor plasma samples both before Ab6 administration and one-hour following Ab6 administration. PF4 levels were analyzed either at baseline or 1 hour after administration as quality control for platelet activation in each sample (FIG. 22). General methods for determining circulating latent TGFβ levels are provided in Example 3, above. Circulating TGFβ levels were measured at additional time points following Ab6 administration and results are shown in FIGS. 23A-C. Tables 16A-C below show exemplary baseline TGFβ and PF4 concentrations from plasma samples collected from patients treated with Ab6.

TABLE 16A
Circulatory TGFb1 level at pre-dose(pg/ml)

| | Patient | Patient |
|---|---|---|
| 80 mg to 240 mg Ab6 | 101-001 | 104-003 |
| Baseline TGFβ1 (pg/ml) | 3920 | 6060 (1 hr) |
| PF4 (pg/ml) | 266 | 451 |

TABLE 16B
Circulatory TGFb1 level at predose(pg/ml)

| | Patient | Patient | Patient |
|---|---|---|---|
| 800 mg Ab6 | 101-002 | 104-001 | 105-002 |
| Baseline TGFβ1 (pg/ml) | 2700 | 3780 | 5480 |
| PF4 (pg/ml) | 82.3 | 277 | 258 |

TABLE 16C
Circulatory TGFb1 level at predose(pg/ml)

| | Patient | Patient | Patient | Patient | Patient |
|---|---|---|---|---|---|
| 1600 mg | 101-003 | 101-004 | 103-004 | 104-002 | 105-003 |
| Baseline TGFβ1 | 4690 | 5790 | 7160 | 2850 | 3220 |
| (pg/ml) | | | (1 hr) | | |
| PF4 (pg/ml) | 145 | 223 | 442 | 192 | 113 |

### Example 6: Measurement of Latent TGFβ Activation

Inhibitory activity of Ab6 was measured as previously described in Martin et al., 2020. Briefly, LN229 cells (ATCC) were transfected with a plasmid encoding either human, rat, or cynomolgus macaque proTGFβ1. About 24 hours after cell transfection, Ab6 was added to the transfectants together with CAGA12 reporter cells (Promega, Madison, WI). Approximately 16-20 hours after setting up the co-culture, the assay was developed and luminescence read out on a plate reader. Dose-response activities were nonlinearly fit to a three-parameter log inhibitor vs. response model using Prism 8 and best-fit IC50 values calculated.

Modulation of TGFβ signaling in Ab6-treated tumors will be detected using a P-Smad 2 IHC assay, where decreased expression in P-Smad2 is indicative of TGFβ inhibition. As shown in FIG. 24, a P-Smad2 IHC assay was developed using commercially available melanoma samples and a digital nuclear masking analysis. Nucleus mask is a digital image analysis parameter that enables visualization and measurement of IHC signal intensity of an individually marked cell or nucleus. The assay was established using the commercial melanoma samples to enable identification of a range of P-Smad2 nucleus staining intensity ranging from high (red), medium (orange), low (yellow) to negative (blue). Cells were sorted into categories (e.g., 0, 1+, 2+, 3+) based on chromogenic intensity relative to scanning parameters. Variables analyzed during assay development are shown in Table 17 below. Analysis was carried out using software developed by Flagship Biosciences.

TABLE 17
Primary Data Variables for P-Smad 2 IHC Assay

| Variable Name | Description |
|---|---|
| Total Number of Quantified | Total number of cells quantified by |
| Cells | IA in compartment |
| Percent Total Cells | Percent of Cells in compartment out |
| | of All Cells |
| Number of p-Smad2 positive | Number of p-Smad2 positive cells |
| Cells | in compartment |
| Number of p-Smad2 negative | Number of p-Smad2 negative cells |
| Cells | in compartment |
| Percent Positive Cells | Percent of p-Smad2 in the compartment |
| | identified |
| Compartment Area (mm²) | Area of compartment in mm² |
| Compartment Area (Percent) | Percent of the total tissue area |
| | occupied by compartmental area |
| %0 p-Smad2 cells | Percent of cells at 0 intensity |
| %1 + p-Smad2 cells | Percent of cells at 1 + intensity |
| %2 + p-Smad2 cells | Percent of cells at 2 + intensity |
| %3 + p-Smad2 cells | Percent of cells at 3 + intensity |
| Number of 0 p-Smad2 cells | Number of cells at 0 intensity |
| Number of 1 + p-Smad2 cells | Number of cells at 1 + intensity |
| Number of 2 + p-Smad2 cells | Number of cells at 2 + intensity |
| Number of 3 + p-Smad2 cells | Number of cells at 3 + intensity |
| H-Score | Standard scoring paradigm representing |
| | both the prevalence and intensity of |
| | biomarker expression. H-Score scale |
| | ranges from 0-300. |

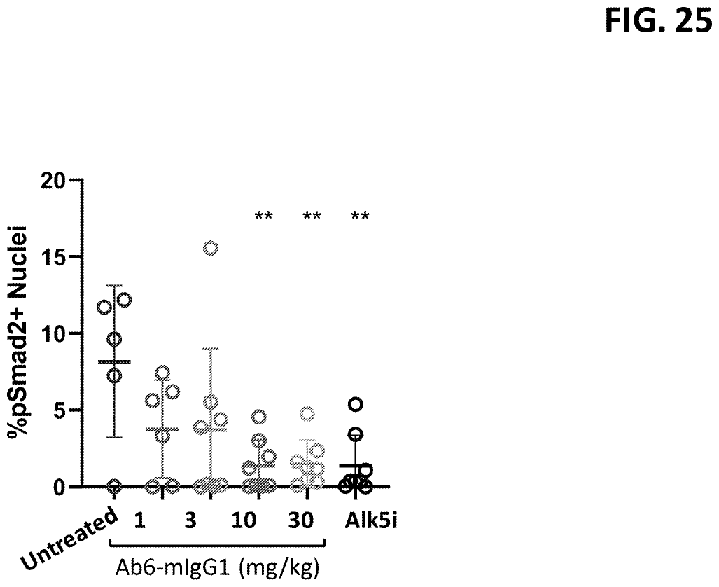

pSmad2 phosphorylation was measured in MBT2 mice, which further supported the rationale for measuring tumor phosphorylated smad 2 (p-Smad2) as a biomarker for SRK-181-mediated TGFβ1 inhibition. As shown in FIG. 25, p-Smad2 levels were analyzed in MBT-2 mice (bladder cancer) at day 10 following weekly dosing of Ab6-mIgG1 at 1 mg/kg, 3 mg/kg, 10 mg/kg, or 30 mg/kg. Ab6-mIgG1 treatment reduced tumor p-Smad2 phosphorylation, thereby supporting p-Smad2 as a pharmacodynamic biomarker.

Briefly, samples from MBT-2 mice were fixed, processed, embedded, and sectioned at 4 μm thickness. Slides were deparaffinized and stained using a Leica Bond Rx autostainer. Sections were blocked using a 1× diluted Animal Free Blocking solution from Cell Signaling Technologies (#15019). Staining was performed using a phospho-Smad2 rabbit monoclonal antibody from Cell Signaling Technologies (#3108, 138D4) at a final concentration of 0.174 ug/mL. Heat induced epitope retrieval was performed using a citrate-based solution for 20 minutes at 95° C. Polymer-Rabbit HRP was incubated as a post-primary for 25 minutes, procured from Cell Signaling Technologies (#8114S). Secondary fluorophores, spectral DAPI, and Tyramide Signal Amplification reagents were procured from Akoya Biosciences (NEL741001KT, FP1490). Sections were dried and cover-slipped with antifade mounting media (ThermoFisher, P36961), and imaged on a Zeiss AxioScan z1 with an additional Cy5 band to further separate autofluorescence signal.

Image quantification was performed using a Visiopharm analysis platform. Nuclei were segmented first on the basis of DAPI staining intensity and resulting objects were reclassified as pSmad2 strong, medium, or weakly positive. These classifications were thresholded by FITC intensity, with a background threshold of 15000 ABU and requiring at least 30% nuclear coverage. Segmented objects with area less than 23 um2 or greater than 230 um2 were discarded before pSmad2 classifications were applied. Strong, medium, and weak classifications were used for QC purposes, and total % pSmad2 positivity is reported as the sum of these groups, divided by the total number of cells, and multiplied by 100.

### Example 7: Analysis of CD8-Positive Cells in Ab6-Treated Tumors

Investigation of CD8 T cell status in biopsied tissues typically describes each tissue as one of three main phenotypes: immune desert, immune-excluded, or inflamed. Immune desert phenotypes do not express appreciable levels of CD8 throughout the tissue. Immune-excluded tissues exhibit CD8 expression, but the expression is mostly localized to the stroma or the stromal margin surrounding tumor nests. Inflamed tissues show appreciable levels of CD8 expression or CD8-positive cells within the tumor nests of the tissues. While this phenotypic categorization is beneficial, these percentages of expression are often calculated as a mean of expression through the tissue and does not take in to account the heterogeneous nature of tumor biology. This may result in a tumor containing one highly inflamed tumor nest being averaged out with multiple deserted tumor nests to categorize the tissue as excluded or deserted even though inflammation is present.

To better represent the heterogeneity of inflammation present within tumor tissues, an image analysis-based algorithm was developed which not only separated out the tumor, stroma, and tumor/stroma margin, but identified each tumor nest within the tissue as its own discrete object. This analysis allowed for the enumeration of number and size of all tumor nests within the tissue, and further quantified the percentage of CD8 expression within and outside of each tumor nest. Each tumor nest was given its own phenotypic classification of inflamed, excluded, or deserted, and the percentage of tumor nests displaying each phenotype was calculated to represent the heterogenic inflammation within tumors.

Core needle biopsy was used to obtain tumor samples from 36 subjects diagnosed with bladder cancer or melanoma. Three to four core biopsy samples were collected from each tumor using a 16- to 18-gauge needle. Samples were fixed at room temperature in a formalin container for 24 to 48 hours. Once fixation was completed, biopsies were transferred to a histocassette between sponges pre-soaked with PBS. The histocassette was then submerged in cold PBS and stored for no more than 3-4 days prior to analysis.

The percentage of CD8+ cells was determined by immunohistochemical analysis in 23 whole-tissue tumor

| | | | | | |
|---|---|---|---|---|---|
| Sample | Tumor Nest | Tumor Nest Total | Tumor Nest CD8 | Interface CD8 | Tumor Nest |
| Name | Number | Cell Count | % Positive | % Positive | Status |

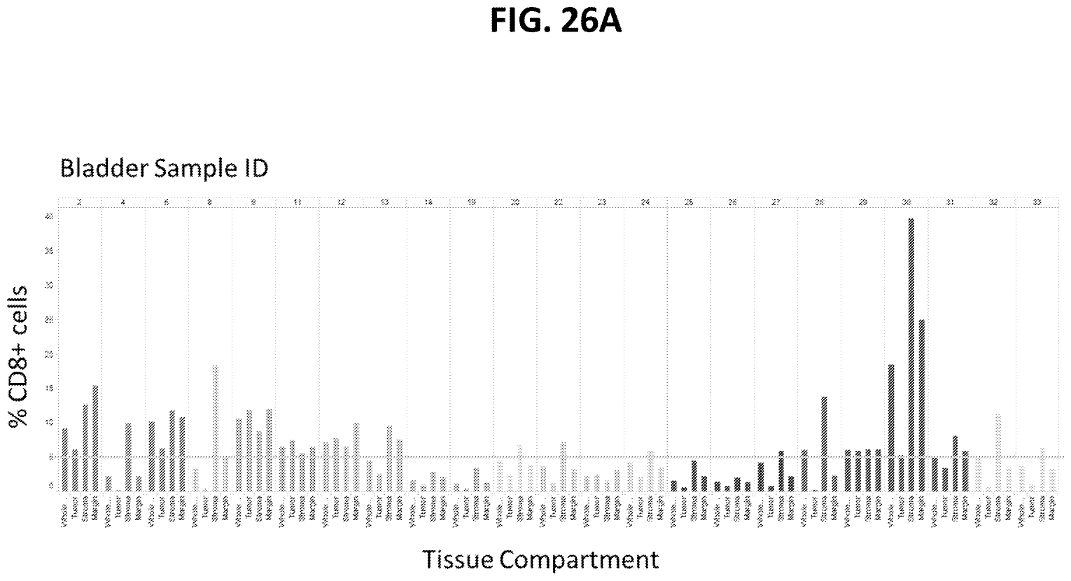

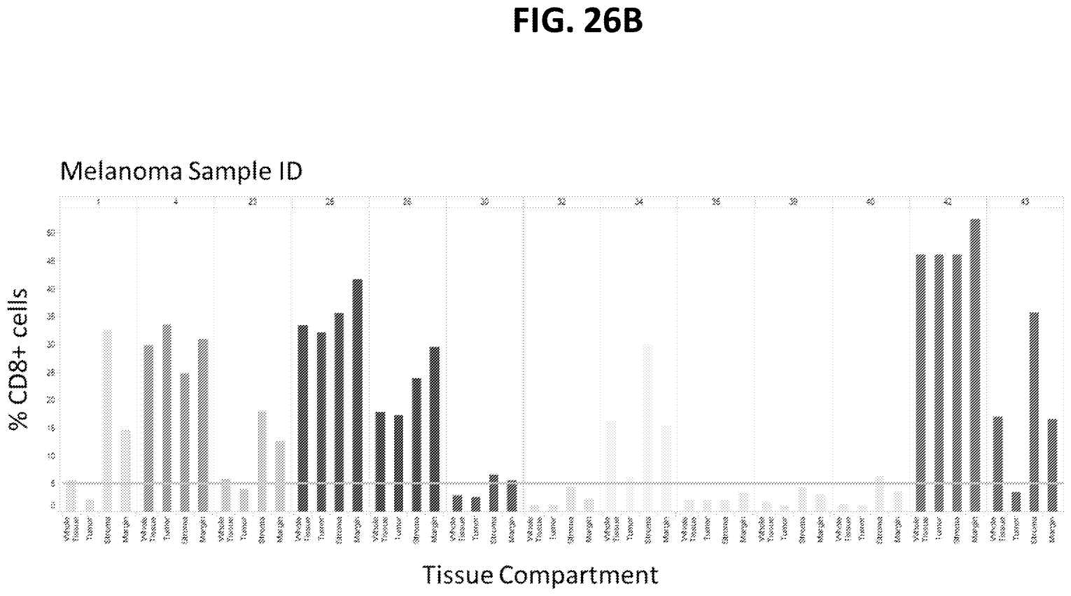

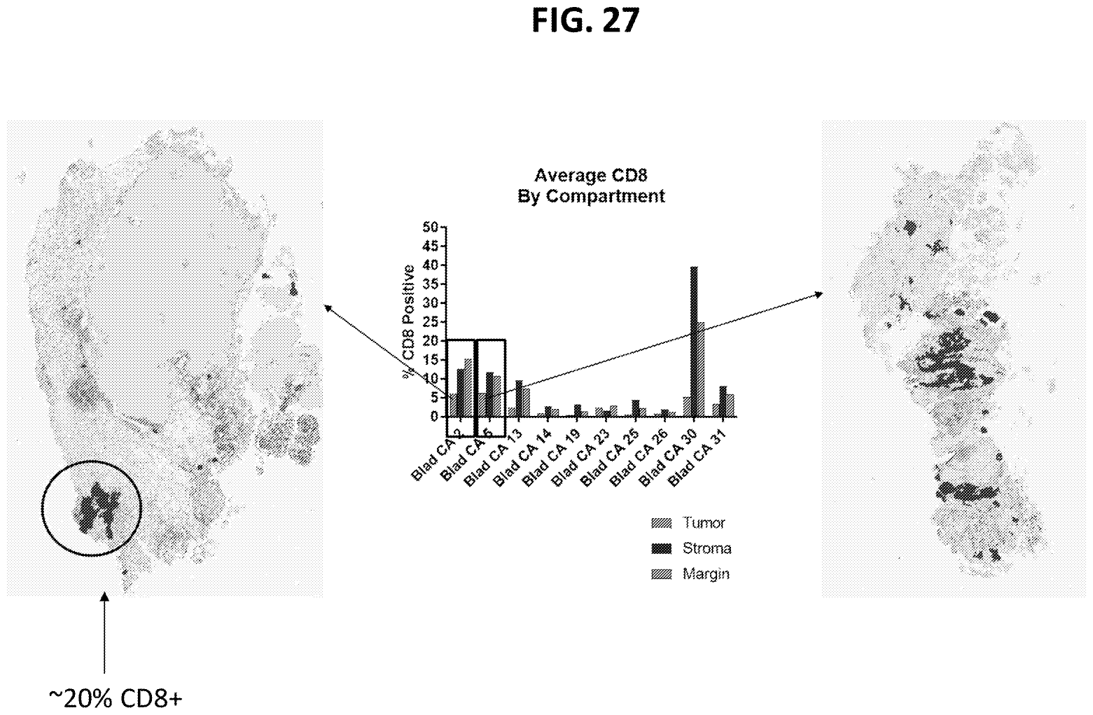

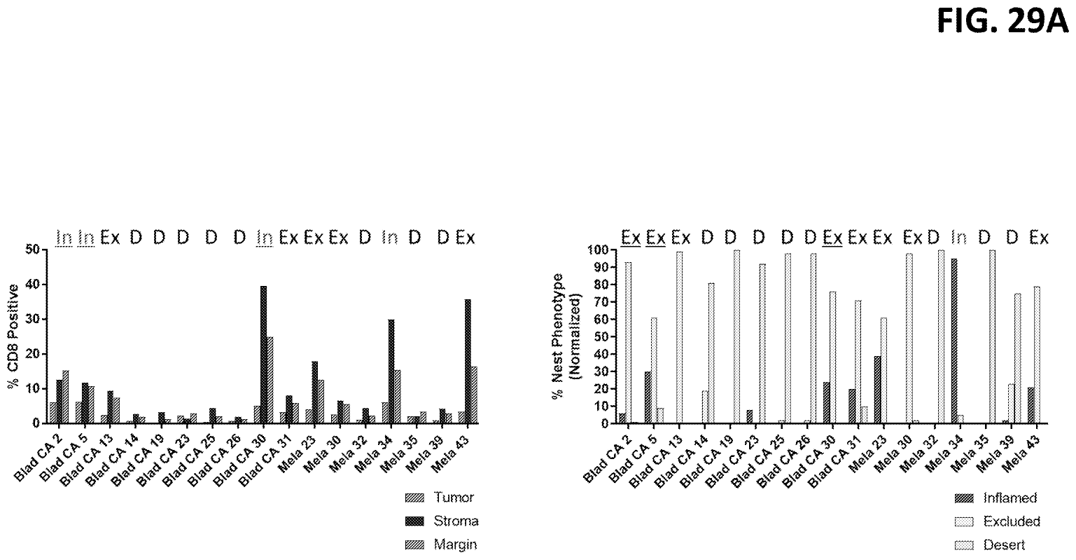

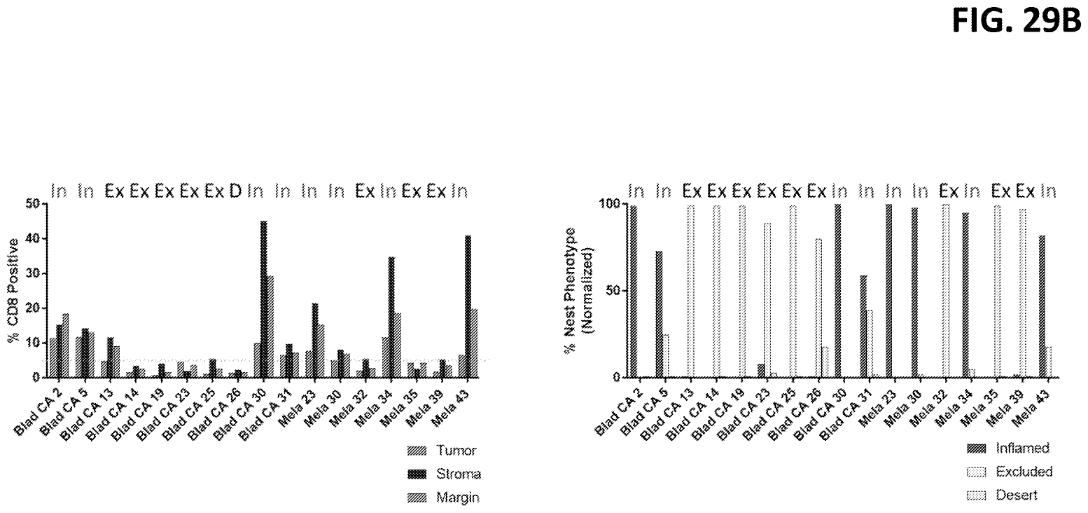

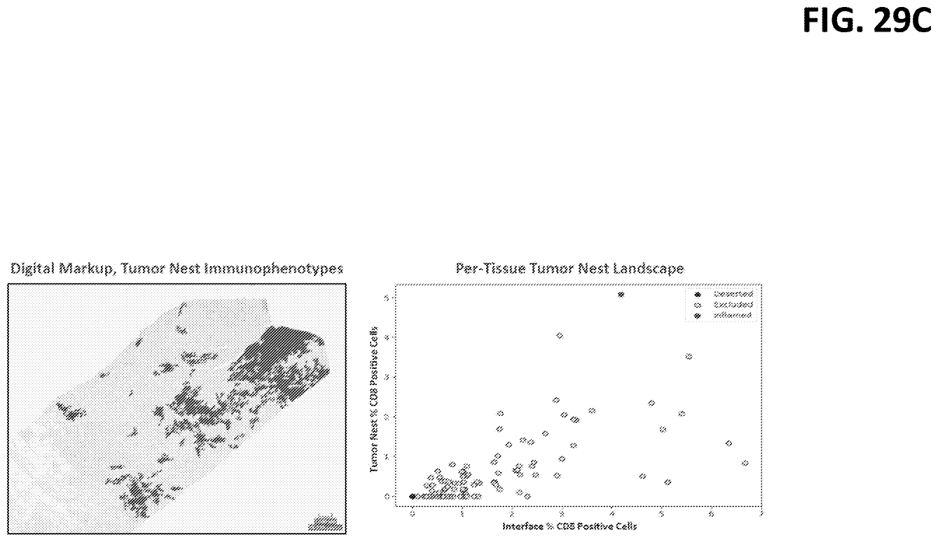

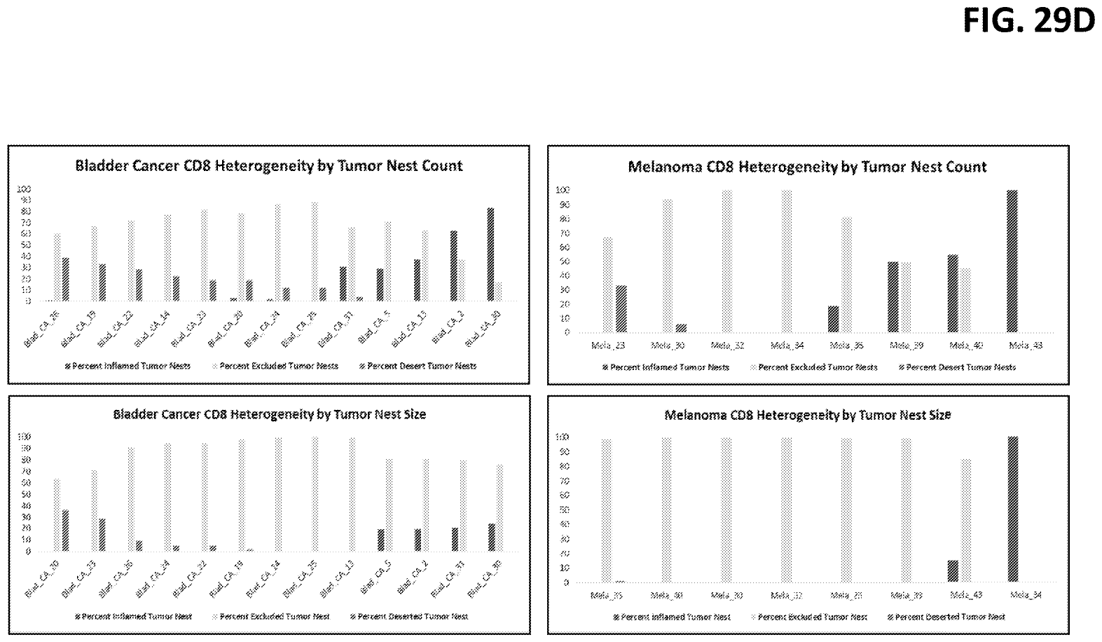

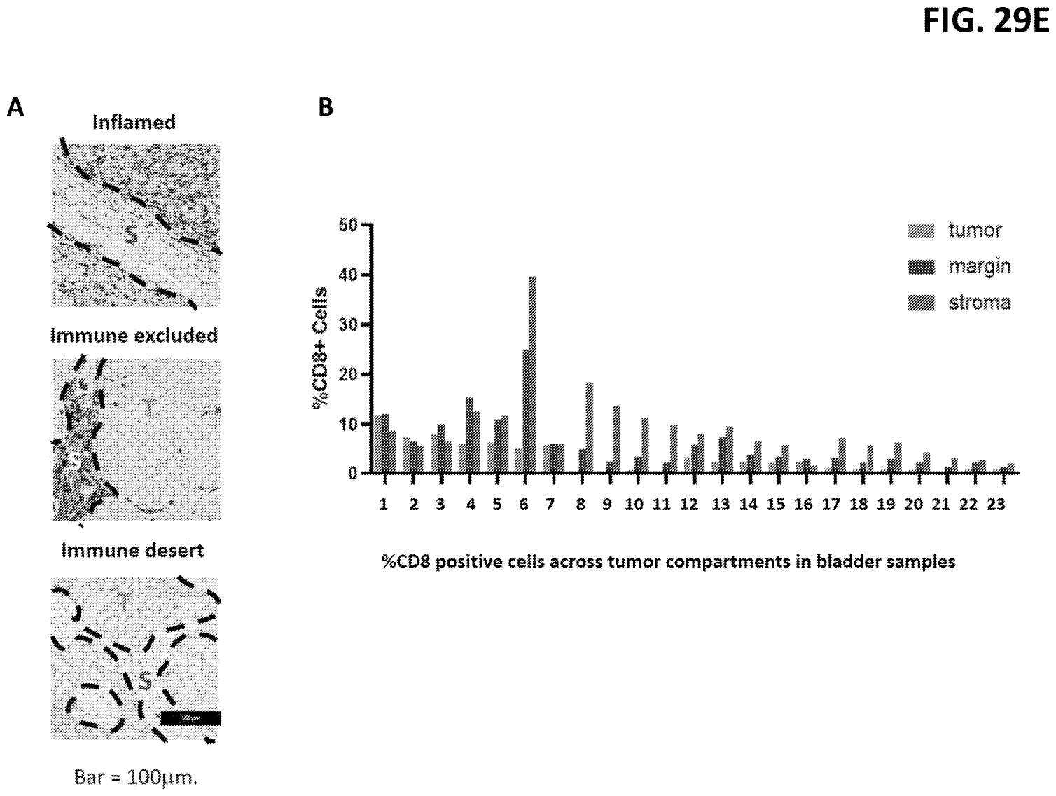

resection samples of bladder cancer and 13 samples of melanoma. Data from the whole tissue, as well as tumor, stroma, and margin (25 μm in each direction from the tumor/stromal interface) compartments of the whole tissue, were evaluated. Margins between tumor nests and the surrounding stroma and margin compartments were identified by digital analysis of the stained samples using pathologist assessment and automated machine learning software developed by Flaship Biosciences (Westminster, CO). Percentage of CD8+ cells was also evaluated for individual tumor nests throughout samples that contained tumor nests with potentially mixed immunophenotypes. For samples which were poorly-defined or distinctly lacked analyzable tumor, only whole tissue was analyzed. Results from compartment analysis demonstrated variation in the percentage of CD8+ cells among different compartments in bladder cancer (FIG. 26A) and melanoma (FIG. 26B) samples. Cell counts, compartmental area, CD8+ cell density (average number of CD8+ cells/mm²), and CD8+ cell clustering were measured. Results from tumor nest analysis are shown in FIGS. 27-29E. Cell count based on FIG. 29C is provided below.

TABLE 18

| | | | | | |
|---|---|---|---|---|---|
| BladderCA 26 | 1 | 281 | 0 | 2.308 | Excluded |
| BladderCA 26 | 2 | 585 | 0.513 | 2.899 | Excluded |
| BladderCA 26 | 3 | 703 | 2.418 | 2.883 | Excluded |
| BladderCA 26 | 4 | 256 | 5.078 | 4.181 | Inflamed |
| . . . | . . . | . . . | . . . | . . . | . . . |
| BladderCA 26 | 84 | 405 | 0 | 0 | Immune |
| | | | | | Desert |
| BladderCA 26 | 85 | 420 | 0.238 | 0.716 | Excluded |
| BladderCA 26 | 86 | 250 | 0 | 0 | Immune |
| | | | | | Desert |
| BladderCA 26 | 87 | 780 | 0 | 0.703 | Excluded |
| BladderCA 26 | 88 | 1196 | 0.0836 | 0.536 | Excluded |
| BladderCA 26 | 89 | 452 | 0 | 0.287 | Excluded |
| BladderCA 26 | 90 | 445 | 4.045 | 2.953 | Excluded |
| BladderCA 26 | 91 | 527 | 0 | 0.623 | Excluded |
| BladderCA 26 | 92 | 1068 | 0 | 0.109 | Excluded |
| BladderCA 26 | 93 | 910 | 0.33 | 0.931 | Excluded |
| BladderCA 26 | 94 | 761 | 0.657 | 2.111 | Excluded |
| BladderCA 26 | 95 | 293 | 0.341 | 1.636 | Excluded |
| BladderCA 26 | 96 | 336 | 2.083 | 5.398 | Excluded |
| BladderCA 26 | 97 | 395 | 0.759 | 2.143 | Excluded |
| BladderCA 26 | 98 | 352 | 0.284 | 1.674 | Excluded |

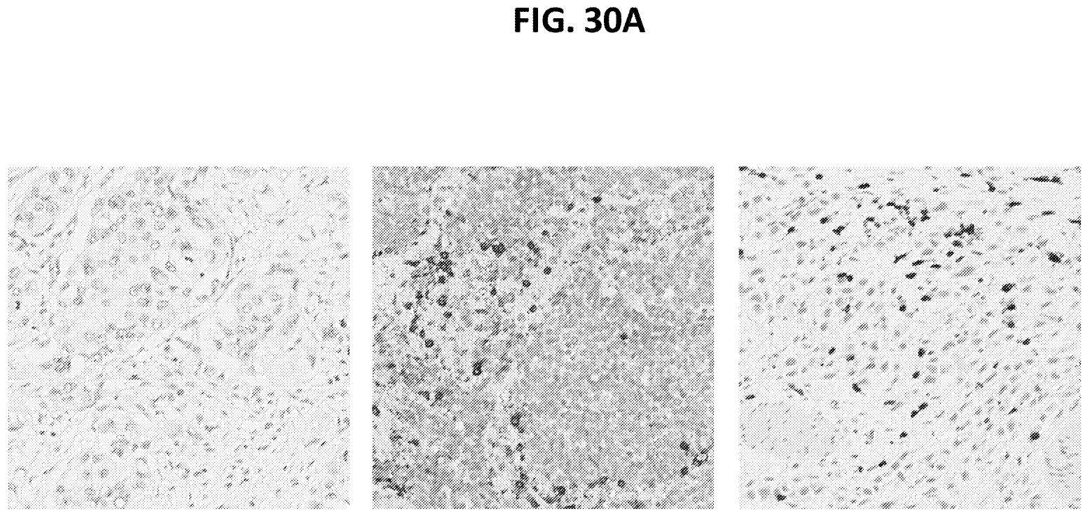

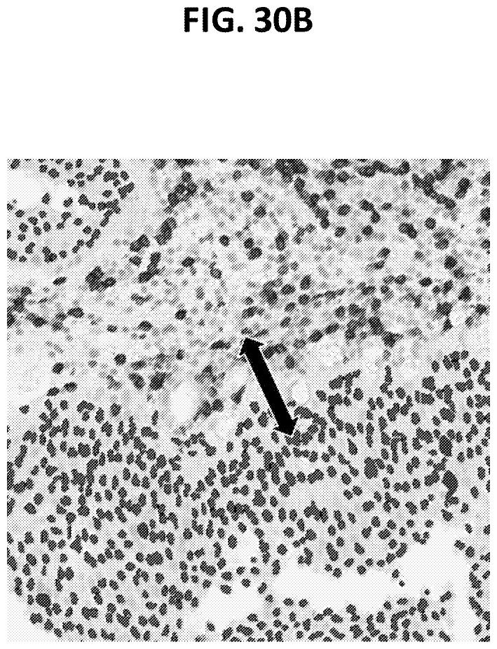

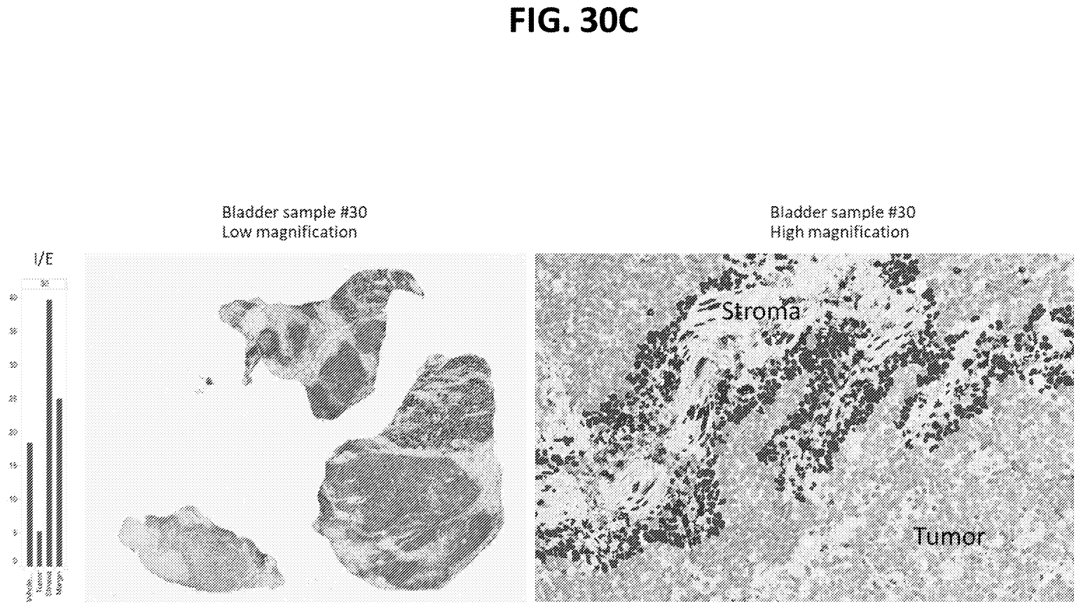

To determine the immune phenotype, the percentage of CD8+ cells in the tumor compartment was compared to that of the stromal and margin compartment. The ratio of CD8+ cells in the tumor compartment to that of the stromal or margin compartments varied across immune phenotypes. As an example, FIG. 30A shows that bladder cancer samples #26, #30, and #9 exhibited different percentages of CD8+ cells across compartments, which indicated that these tumors likely had different immune phenotypes. Bladder sample #26 (FIG. 30A, left) exhibited an immune desert phenotype, as demonstrated by low CD8+ staining across all three compartments (0.8% CD8+ staining in the tumor, 1.9% in the stroma, and 1.3% in the margin). Bladder sample #9 (FIG. 30A, right), which exhibited an immune inflamed phenotype, showed similarly high percentages of CD8+ staining across all three compartments (11% CD8+ staining in the tumor, 8.7% in the stroma, and 12% in the margin). In contrast, bladder sample #30 (FIG. 30A, middle), which exhibited an immune excluded phenotype, showed greater percentages of CD8+ cells in the stroma and margin as compared to the tumor (5.2% CD8+ staining in the tumor, compared to 39% in the stroma and 24% in the margin). In some cases, further analysis of CD8+ expressing in the stroma and margin, by subdividing the margin compartment, may provide even more information for immune phenotyping. For instance, as shown in FIG. 30B, 18.3% and 4.8% of CD8+ cells outside the tumor are located in the stroma and margin compartments, respectively, and subdividing the margin component further reveals that nearly all of the CD8+ cells in the margin lie on the stromal-facing side of the margin with almost no CD8+ cells found in the tumor-facing side. Similarly, FIG. 30C shows strong CD8 staining in the tumor margin of bladder sample #30, and subdividing the margin component further demonstrates that nearly all of the CD8 positivity is localized on the stromal side of the margin compartment and nearly no CD8 positivity is localized on the tumor side. These observations indicate that the tumor is likely immune excluded.

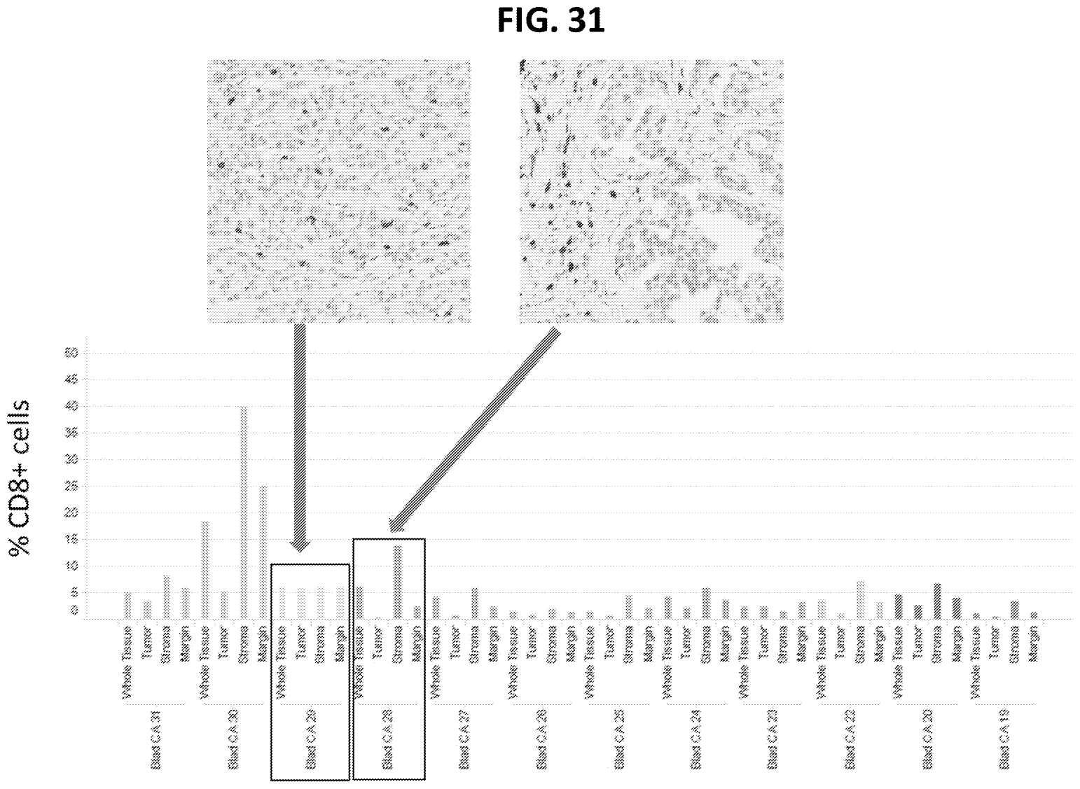

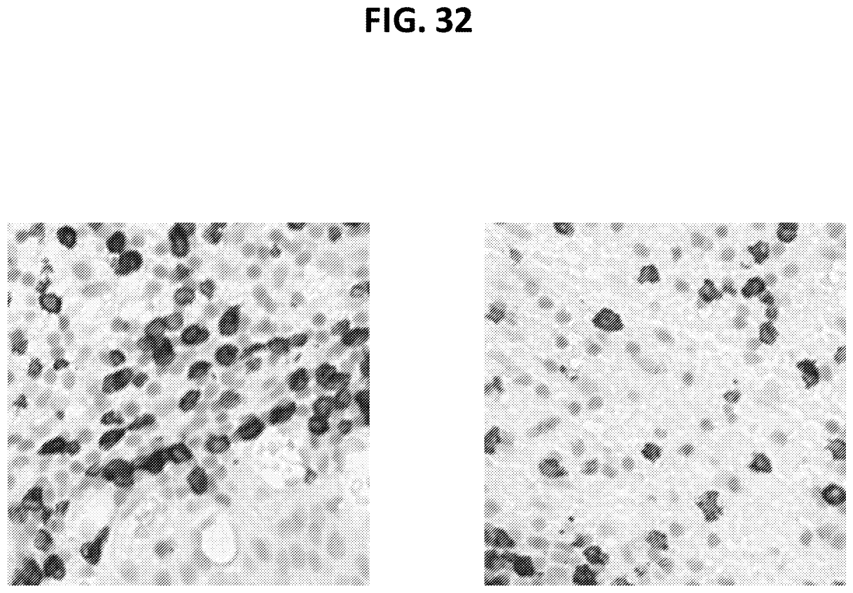

Compartment ratios of CD8+ expression for the tumor, stroma, and margin were compared to absolute percent CD8 positivity in whole tissue. As demonstrated in FIG. 31, tumors that exhibit similar percentages of CD8+ cells display distinct CD8+ expression profiles in each tumor compartment, suggesting that compartment ratios of CD8+ expression may provide more information for immune phenotyping as compared to absolute percent CD8 positivity data of the whole tumor alone. Likewise, CD8+ cell density in each tumor compartment, as determined by the number of CD8+ cells per millimeter squared, was compared to percent CD8+ expression of whole tissue. IHC staining data in FIG. 32 show two different tumor stroma sections have an approximately 10-fold difference in CD8+ cell densities despite exhibiting similar overall percentages of CD8+ expression (FIG. 32). These findings suggest that cell density may be used as an additional or alternative measurement to absolute CD8 positivity, and that in some cases, cell density data may better represent tumor immune populations for immune phenotyping.

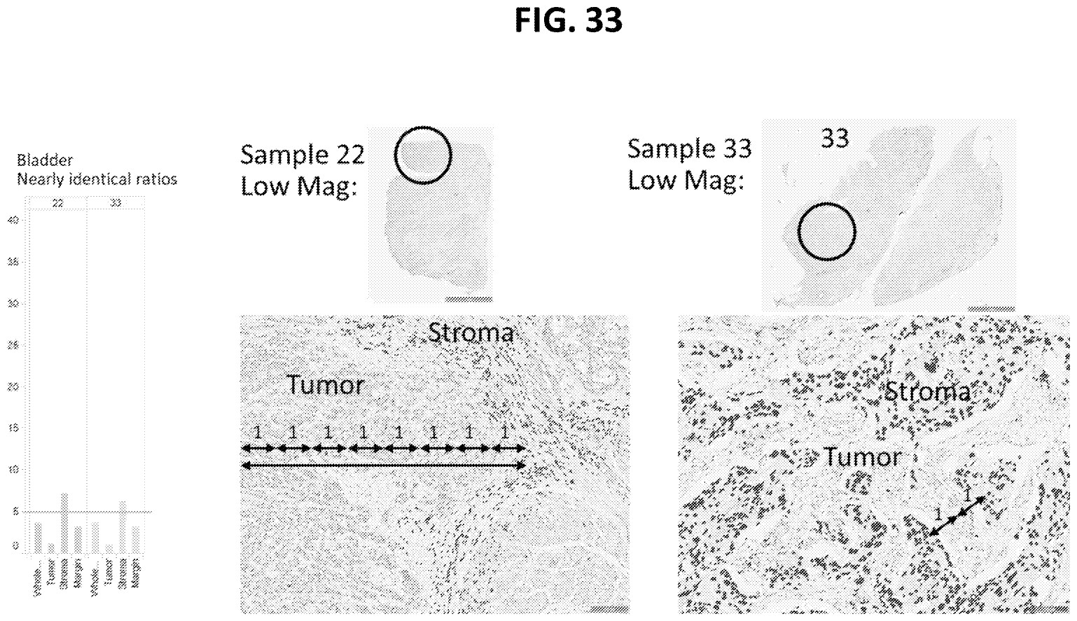

Tumor depth was determined for two bladder samples by determining the distance available for CD8+ T cell penetration in the tumor nest of a given sample. Bladder sample #22 had a tumor depth of greater than 8 (measurement continued toward opposite side of the tumor nest), whereas bladder sample #30 had a tumor depth of less than 2 (FIG. 33). Given that the percent CD8 expression in these samples were found to be similar, these results suggest that tumor depth may be a useful parameter for determining/confirming tumor immunophenotyping when used in combination with other parameters such as percent CD8 expression.

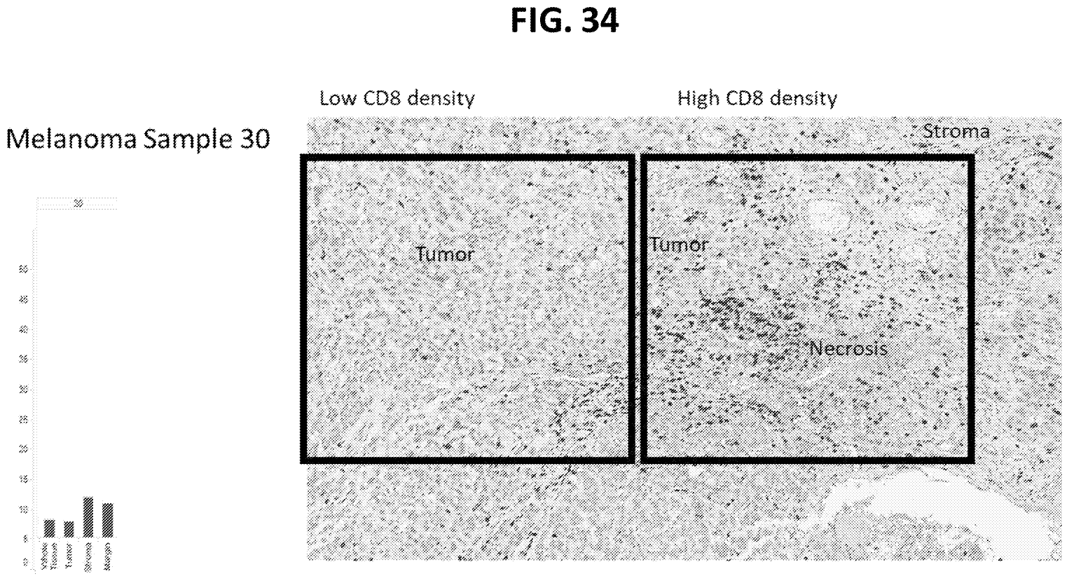

Localized CD8 expression was further analyzed for melanoma sample #30, which showed low overall CD8 percentages. Localized areas of CD8 expression showed a concentration of CD8 cells near necrotic regions, which may be indicative of potential treatment effects (FIG. 34).

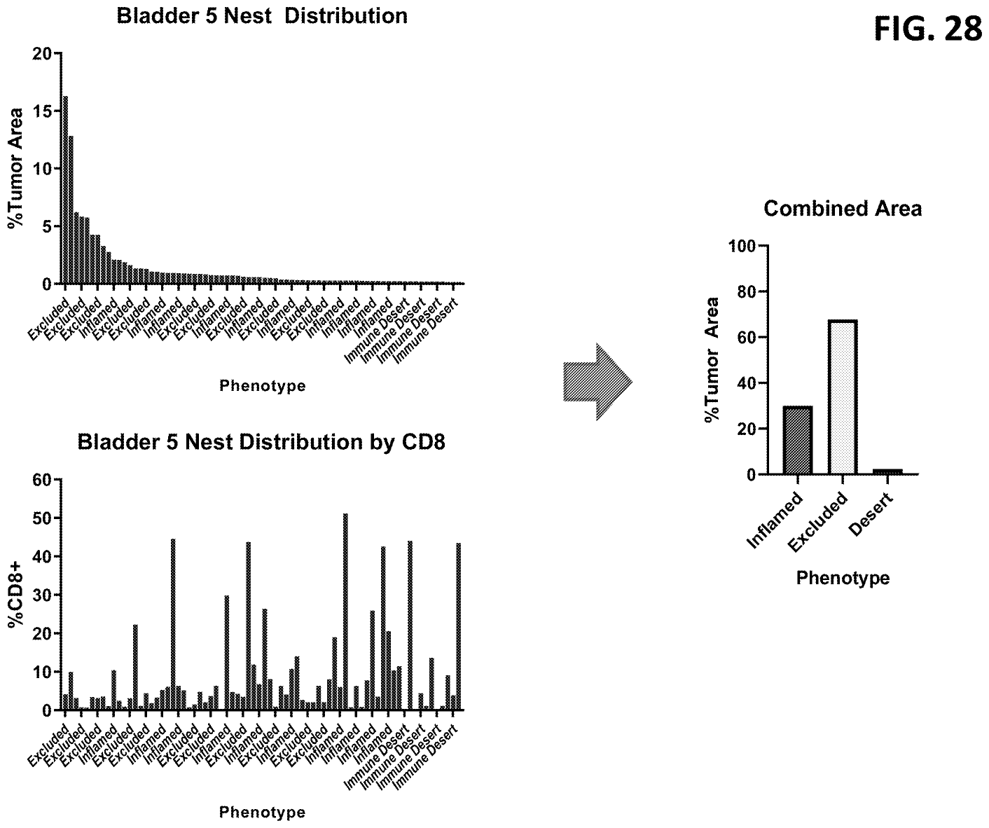

CD8 positivity (e.g., percentage of CD8+ cells) was determined for individual tumor nests and compared to CD8 positivity as measured in tumor compartments. As shown in FIG. 27, further breakdown of tumor compartments into tumor nests reveals varying degrees of CD8 positivity in different parts of the tumor and provides additional insight into the immune phenotype of the tumor. For instance, the immune phenotype of a bladder tumor sample was determined by first measuring the CD8 positivity of 74 tumor nests, then assigning each individual tumor nest a relative phenotype (e.g., immune inflamed, immune excluded, or immune desert) based on CD8 positivity, and finally calculating the combined tumor areas exhibiting each immune phenotype (FIG. 28). In some cases, immune phenotypes as determined based on tumor nest analysis differed from the immune phenotypes determined based on analysis of tumor compartments alone (FIGS. 29A-E).

### Example 7: Prevalence of Tumor MDSC in Various Solid Tumors

A signal intensity filter for detecting cell surface markers of human MDSCs was established. Multiple markers were selected and developed to distinguish MDSC subtypes from other monocytes. Cell surface markers for distinguishing human MDSCs include CD11 b, CD33, CD66b, CD14, CD15, and HLA-DR. A chromogenic assay was performed to define the immunofluorescence dynamic range of each antibody used to detect the cell surface markers. Two types of signal intensity filters were used to identify MDSCs: 1) binary intensity selection, i.e., based on positive vs. negative signal, e.g., distinguishing CD14+ mMDSC from CD15+ gMDSC; and 2) categorical binning of signal intensities to define a range of signal intensities, e.g., to distinguish HLA-DR^{low-neg}mMDSC from HLA-DR^{neg}gMDSC. Signal intensity filters were applied sequentially, and categorization was reviewed and confirmed by pathologists. Exemplary thresholds for detecting cell surface markers after application of signal intensity filters are shown in Tables 19A-C below.

TABLE 19A

| | HLADR | CD33 |
|---|---|---|
| Negative | <0.105 | <0.17 |
| Low | 0.105-0.125 | 0.17-0.18 |
| Med | 0.125-0.155 | 0.18-0.19 |
| High | >0.155 | >0.19 |

TABLE 19B

| | CD11b | CD14 | CD15 | CD66b |
|---|---|---|---|---|
| Negative | <0.145 | <0.24 | <0.19 | <0.19 |
| Positive | >0.145 | >0.24 | >0.19 | >0.19 |

TABLE 19C

| | CD11b | HLADR | CD14 | CD15 | CD33 | CD66b |
|---|---|---|---|---|---|---|
| gMDSC | + | − | − | + | +/low | + |
| mMDSC | + | −/low | + | − | +/high | − |
| classical monocyte | + | + | + | − | + | − |
| neutrophil | + | −/med | − | + | +/med | + |
| m1 macrophage | + | +/high | − | − | + | − |
| m2 macrophage | + | + | − | − | + | − |

An exemplary analysis of signal intensity filtering of MDSC markers in bladder cancer is shown in FIG. 35A. An example of HLA-DR detection using threshold cutoffs is shown in FIG. 35B. All cells were plotted to illustrate threshold cuttoffs, where the x-axis shows staining intensity (binned columns) and the y-axis shows the number of cells. Visualization of signal intensities were normalized by channel in order to reduce background and bleed through (spectral unmixing). Because of this, threshold cutoffs varied from channel to channel. Examples of signal normalization are shown in the panels of FIG. 35C.

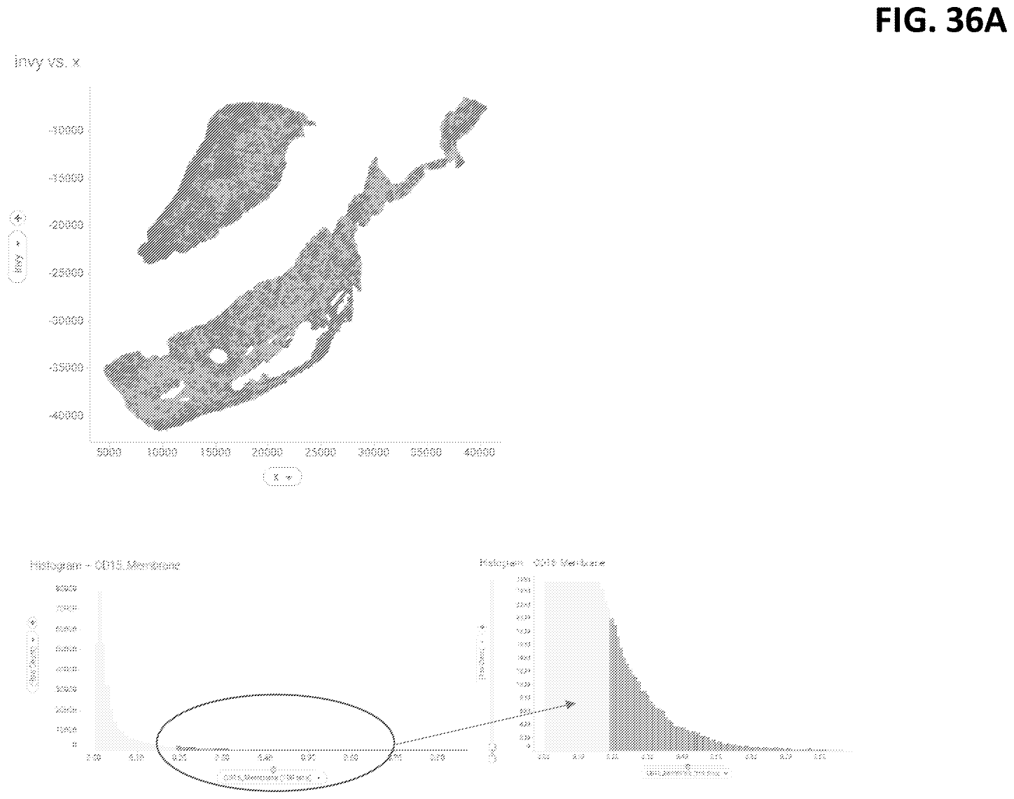

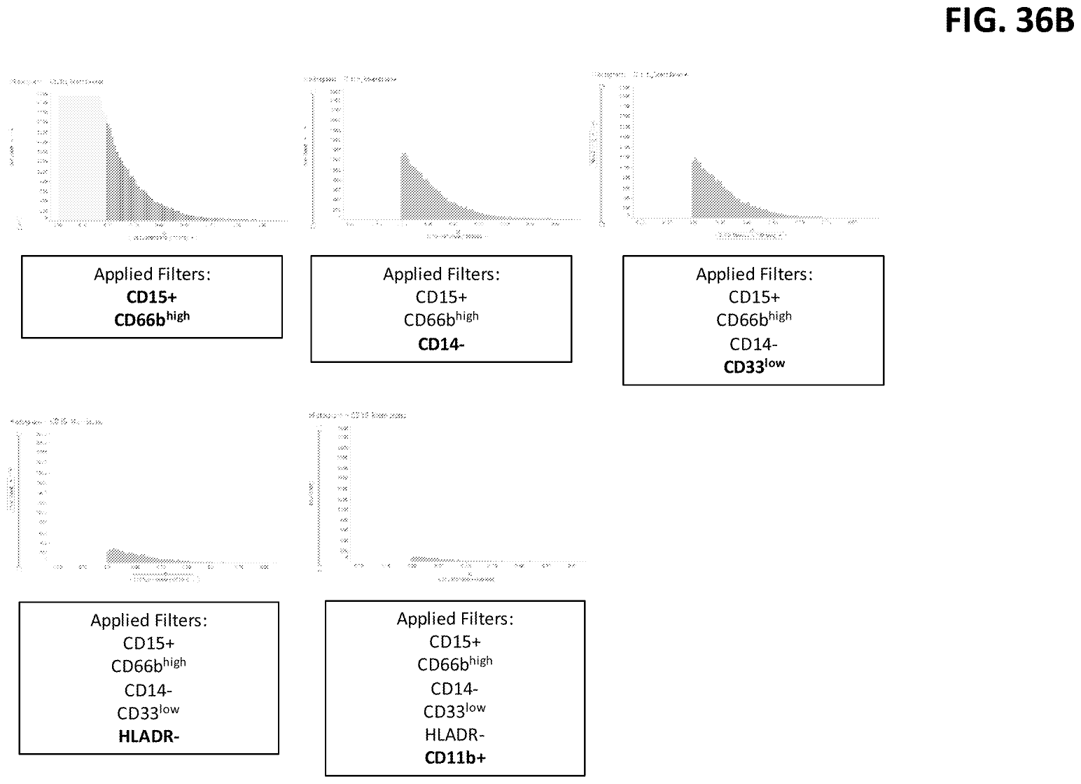

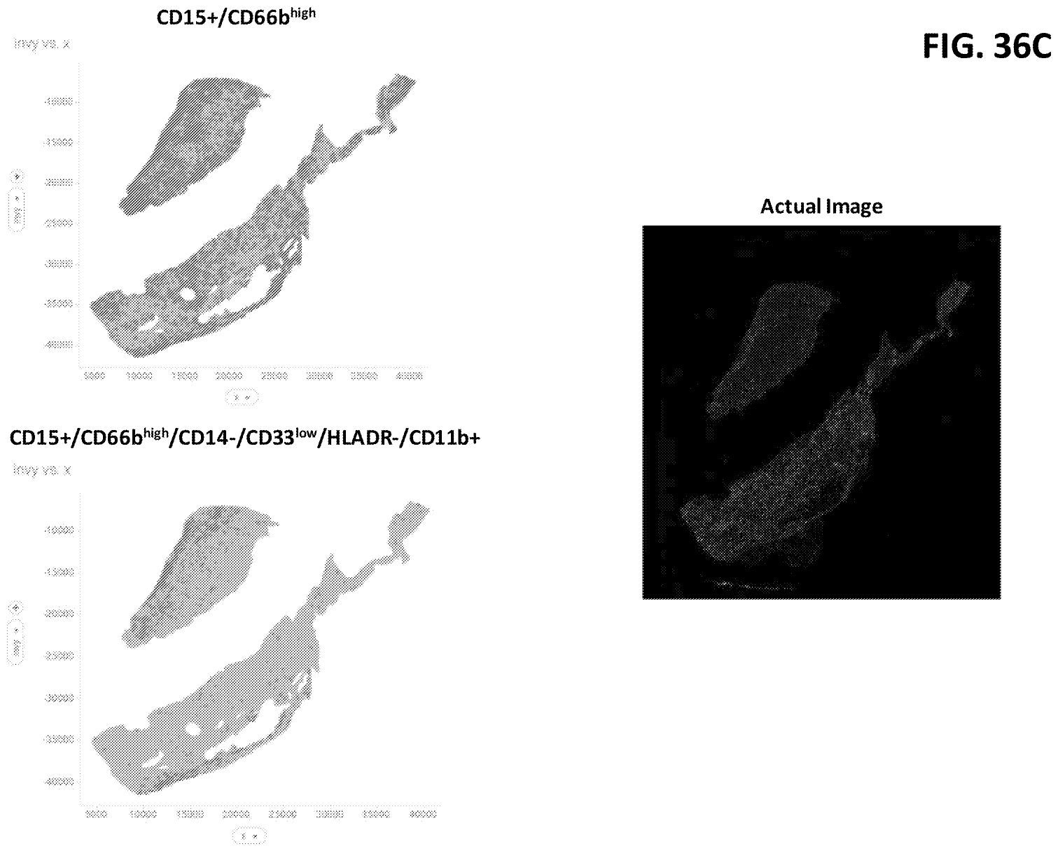

gMDSC populations in tumor samples were identified by first filtering for CD15 and CD66b positivity (FIG. 36A). This filtering distinguished gMDSCs from mMDSCs, which are CD15 and CD66b negative. Sequential application of signal filters was then applied to specify gMDSCs according to the markers and thresholds described above (FIG. 36B). A comparison of the original tumor image from a bladder cancer sample (right panel), without signal filtering, the image after applying CD15+/CD66b^{high} filtering (top left panel), and the image after applying CD15⁺/CD66b^{high}/CD14⁻/CD33^{low}/HLADR⁻/CD11b⁺filtering (bottom left panel) is shown in FIG. 36C. A large portion of the gMDSC population appeared to be at the tumor edge, near the stromal compartment.

Using the same signal filtering described above, gMDSC and mMDSC populations were analyzed in samples from bladder cancer, melanoma, NSCLC, and ovarian cancer (FIGS. 37A-C). The samples had much higher gMDSC populations as compared to mMDSC populations. Ovarian cancer exhibited the lowest gMDSC levels and the highest mMDSC levels among the various indications examined. mMDSC levels were the lowest in melanoma samples.

### Example 8: Phase 1 Clinical Study

A multi-center, open-label, Phase 1, first-in-human, dose escalation and dose expansion study (DRAGON; NCT04291079) is ongoing to evaluate the safety, tolerability, pharmacokinetics, pharmacodynamics, and efficacy of Ab6 administered alone and in combination with anti-PD-(L)1 therapy in adult patients with locally advanced or metastatic solid tumors. The study is divided into three treatment parts, Part A1, Part A2, and Part B, and a Long-Term Extension Phase (LTEP). Part A1 (dose escalation) determines the maximum tolerated dose (MTD) or maximum administered dose (MAD) of Ab6 as a single agent and the recommended Phase 2 dose (RP2D) of Ab6 as a single-agent. Part A2 (dose escalation) determines the MTD or MAD of Ab6 in combination with anti-PD-(L)1 antibody therapy and the RP2D of Ab6 in combination with anti-PD-(L)1 antibody therapy for use in Part B. In Part B (dose expansion), parallel cohorts of patients with Non-small cell lung cancer (NSCLC), Urothelial Carcinoma (UC), Cutaneous melanoma (MEL), renal cell carcinoma, or another advanced or metastatic solid tumor type that is not NSCLC, UC, or MEL, are enrolled to confirm the tolerability of the RP2D of Ab6 (determined in Part A2) and to evaluate the anti-tumor activity of Ab6 in combination with an anti-PD-(L)1 antibody therapy. Patients in Part A1 may continue treatment with Ab6 as a single agent at the RP2D in the LTEP following 3 cycles of treatment with Ab6 as a single agent in Part A1. Patients in Part A2 may continue treatment with Ab6 at the RP2D in combination with anti-PD-(L)1 antibody therapy in the LTEP following 3 cycles of treatment with Ab6 in combination with anti-PD-(L)1 antibody therapy in Part A2. Patients in Part B may continue treatment with Ab6 in combination with anti-PD-(L)1 antibody therapy in the LTEP following 9 cycles of treatment with Ab6 in combination with anti-PD-(L)1 antibody therapy in Part B.

In Part A1, patients with advanced solid tumors are administered 80 mg, 240 mg, 800 mg, 1600 mg, 2400 mg, or 3000 mg of Ab6 via intravenous infusion once every three weeks. In Part A2, patients who are non-responders to prior anti-PD-(L)1 therapy are administered a combination of an approved anti-PD-(L)1 therapy based on tumor type and Ab6, where Ab6 is administered intravenously at 240 mg, 800 mg, 1600 mg, or 2400 mg once every 3 weeks. In Part B, patients who are non-responders to prior anti-PD-(L)1 therapy are administered a combination of an anti-PD-(L)1 therapy and Ab6 via intravenous infusion. Cohort A of Part B consists of patients with non-small cell lung cancer (NSCLC) who are administered Ab6 in combination with pembrolizumab. Cohort B of Part B consists of patients with urothelial carcinoma (UC) who are administered Ab6 in combination with pembrolizumab. Cohort C of Part B consists of patients with melanoma (MEL) who are administered Ab6 in combination with pembrolizumab. Cohort D of Part B consists of patients with a cancer that is not NSCLC, UC, or MEL, and the patients are administered Ab6 in combination with an approved anti-PD-(L)1 therapy based on tumor type. Blood chemistry assays are performed as a safety assessment in Part B, including measuring blood levels of cytokines interleukin-1 beta (IL-1p), tumor necrosis factor alpha (TNBa), interleukin-6 (IL-6), interleukin-8 (IL-8), interferon gamma (IFNγ), and interleukin 10 (IL-10).

Primary outcome measures for the study include 1) evaluating the safety and tolerability of Ab6 (Part A1) or in combination with an anti-PD-(L)1 therapy (Part A2) and 2) determining the MTD or MAD, RP2D, and dose limiting toxicities (DLTs). DLTs response as assessed in the first 21 days of the study. MTD, MAD, RP2D, and DLTs are assessed using RECIST v1.1 and do not include toxicities clearly related to disease progression and intercurrent illness. Safety endpoints include adverse events, clinical observations (e.g., vital signs, physical examination), laboratory tests, electrocardiogram, and echocardiogram.

Secondary outcome measures of the study include evaluating the pharmacokinetics of Ab6 alone or in combination with an anti-PD-(L)1 therapy. Specific pharmacokinetic parameters include maximum drug concentration (Cₘₐₓ), time to Cₘₐₓ (Tₘₐₓ), last validated plasma concentration (Clast), time to Clast (Tₗₐₛₜ), and half-life (T_{1/2}). Time frame for evaluating pharmacokinetic parameters are cycles 1 and 3, where each cycle is 21 days. The anti-tumor activity of Ab6 alone or in combination with an anti-PD-(L)1 therapy is also evaluated as a secondary outcome. Specific clinical response parameters include objective response, defined as a complete response (CR) or partial response (PR), as determined by RECIST v1.1 or iRECIST v1.1. Other secondary outcome measures include evaluating anti-drug antibodies and biomarkers. Biomarker assessment includes evaluation of the immune landscape (e.g., identify immune-inflamed, immune-excluded, and immune desert tumors) and TGFβ pathway status within the tumor. Orthogonal biomarker strategies are also applied to assess multiple biologically related pathways, such assessments include as tumor-based multiplex immunohistochemistry (IHC), next-generation sequencing, and evaluation of blood-based biomarkers.

Key inclusion criteria for the study include patients who are 18 years of age, with a predicted life expectancy of ≥3 months and patients who have measurable disease per RECIST v1.1 at screening. Additionally, patients must have an Eastern Cooperative Oncology Group performance status (PS) 0-1. To meet eligibility for part A1, patients must have a histologically documented solid tumor that is metastatic or locally advanced, for which standard of care therapy does not exist, has failed in the patient, or is not tolerated by the patient, or for which the patient has been assessed by the Investigator as not being a suitable candidate or otherwise ineligible for the standard of care therapy. To meet eligibility for Part A2 and Part B, patients must have a history of primary anti-PD-(L)1 antibody nonresponse presenting (based upon the Investigator's assessment) either as progressive disease or stable disease (e.g., not improving, but also not worsening, clinically or radiographically) after at least 3 cycles of treatment with an anti-PD-(L)1 antibody therapy (alone or in combination with chemotherapy) approved for that tumor type. Additionally, patients must also have received their most recent dose of anti-PD-(L)1 antibody therapy within 6 months of enrollment (or 9 months for UC cohort) in order to meet eligibility for Part B.

Key exclusion criteria include patients who have ECOG performance status ≥2, concurrent anti-cancer treatment, a history of active metastatic CNS disease, an active or prior history of autoimmune disease, hypersensitive or presence of anti-drug antibodies to anti-PD-(L)1 antibody therapy, and/or concurrent second malignancy. Additional exclusion criteria include patients who have second malignancy at other sites with the exceptions of adequately treated in situ carcinoma, e.g., cervical carcinoma, non-melanoma skin cancer, bilateral synchronous discordance breast cancer, or indolent prostate cancer under observation. A past history of other malignancies are allowed as long as the patient has been free of recurrence for ≥2 years, or if the patient has been treated with curative intent within the past 2 years, and in the opinion of the Investigator, is unlikely to have a recurrence. Women who are pregnant or breastfeeding are also excluded. Exclusion criteria for Part A2 and Part B include patients who are receiving concurrent anticancer treatment, with the exception of an anti-PD-(L)1 antibody therapy for Part A2 or Part B, either approved or investigational, within 28 days prior to administration of Ab6, patients who have received biologic therapy (except for anti-PD-(L)1 antibody therapy for Part A2 or Part B), <28 days prior to administration of Ab6, pateints who have received systemic cytotoxic chemotherapy (except for in combination with anti-PD-(L)1 antibody therapy)<28 days prior to administration of Ab6, patients who have received targeted small molecule therapy within 5 half-lives of the compound prior to administration of Ab6, or pateints who have a history of intolerance or treatment discontinuation due to severe irAE or other adverse reaction from prior anti-PD-(L)1 antibody therapy.

Twenty patients have been dosed (14 patients in Part A1 and 6 patients in Part A2). Dose escalation phase of the study is on-going. Early results from Part A1 showed that escalation of Ab6 from 80 mg to 2400 mg resulted in no DLTs. Similarly, escalation of Ab6 from 240 mg to 800 mg, when dosed in combination with an anti-PD-(L)1 therapy, did not result in DLTs in Part A2. A dose of 3000 mg of Ab6 is currently under evaluation in Part A2.

### Example 9: Effects of TGFβ1 Inhibition on MK Differentiation in Cells Isolated from Myelofibrosis Patients

TGFβ is capable of promoting the proliferation of marrow stromal cells and collagen deposition as well as endothelial cell proliferation thereby promoting microenvironmental changes that resemble those observed in MF bone marrow. Furthermore, increased levels of TGFβ in myelofibrosis patients have been implicated in both the development of anemia and thrombocytopenia as well as disease development in patients with myelofibrosis. Thus, it is hypothesized that treatment with a TGFβ inhibitor may be able to reverse the undesired effects resulting from increased TGFβ levels in myelofibrosis patients and myelofibrotic cancers. Since megakaryocytes (MK) and platelets are the major sources of TGFβ1 (Blood 2007; 110:986-993), the therapeutic potential of the TGFβ1 inhibitor Ab6 is explored by culturing mononuclear cells or CD34+ cells from myelofibrosis patients under conditions that generate megakaryocyte-enriched populations.

Culture conditions that generate MK enriched populations are as described according to Mosoyan et al., (Leukemia. 2017 November; 31(11): 2458-2467, the contents of which are herein incorporated by reference to their entirety). Briefly, cells are cultured using a two-step liquid culture system of either mononuclear cells or CD34+ cells from healthy donors or myelofibrosis patients (MF-MNCs). Cells are suspended in IMDM medium (Invitrogen, Grand Island, NY) supplemented with 1% penicillin/streptomycin, 1% L-Glutamine (Invitrogen, Grand Island, NY), 20 mM p-mercaptoethanol, 1% bovine serum albumin (BSA) Fraction V (Sigma, St. Louis, MO), 30% serum substitute BIT 9500, 100 ng/ml recombinant human stem cell factor (hSCF), 50 nM/ml recombinant human thrombopoietin (hTPO) (R&D Systems, Minneapolis, MN, USA) (lancu-Rubin et al., Exp Hematol. 2012 July; 40(7):564-74, the contents of which are herein incorporated by reference to their entirety). MK colony forming unit (CFU-MK) assays are performed by using the MegaCult System and Detection Kit according to the manufacturer's instructions (Stem Cell Technologies, Vancouver, BC, Canada). Isolation of CD61+MK is performed using an Immunomagnetic Selection Kit as per manufacturer's recommendations (Miltenyi Biotech Inc., Auburn, CA, USA).

MF-MNC or CD34+ cells from healthy individuals or myelofibrosis patients are cultured with SCF and TPO for 7 days, after which the cells were cultured for 2-8 more days with TPO alone. Ab6, a negative control antibody (e.g., isotype control), or the TGFβR1 kinase inhibitor galunisertib is added for 24-72 hours during conditioning periods of cell cultures as described. Conditioning media is collected from each of the cultures and assayed for levels of TGFβ1, TGFβ2, and TGFβ3 by ELISA. The effects of the conditioned media treated with a negative control antibody, Ab6, or galunisertib on normal fibroblasts and endothelial cell proliferation and on collagen deposition are evaluated. Effects of the conditioned media on normal and myelofibrotic CD34+ colony formation in the presence of cytokine combination are also assessed.

To determine the effects of conditioned media treated with negative control, Ab6, or galunisertib on malignant hematopoiesis, hematopoietic colonies cloned from myelofibrotic CD34+ cells are genotyped for myelofibrosis driver mutations. To determine whether TGFβ1 inhibition eliminates downstream effects of excessive TGFβ signaling, target cells such as fibroblasts, endothelial cells and hematopoietic cells are analyzed for SMAD activation.

MK cultures are analyzed using flow cytometry and MK cells are identified based on expression of CD41 and CD42 protein markers. Treatment of MK cultures with up to 100 nM of Ab6 inhibits autocrine TGFβ1 signaling in MKs from MF-MNCs but does not inhibit pSMAD2 activation by recombinant TGFβ1. In patient cell cultures where Smad phosphorylation is demonstrated, TGFβ activation occurs in a cell-autonomous manner. Suppression of phosphorylation by Ab6 confirms that phosphorylation is induced by TGFβ1. Addition of exogenous TGFβ1 growth factor to cell cultures is used to demonstrate Smad signaling competence in cultures.

### Example 10: Exacerbation of ECM Dysregulation in Mice Treated with TGFβ3 Inhibitor

From a safety standpoint, there has been a wide recognition that pan inhibition of TGFβ can cause toxicities, which underscores the fact that no TGFβ inhibitors have been successfully developed to this day. To circumvent potentially dangerous adverse effects, a number of groups have recently turned to identifying inhibitors that target a subset—but not all—of the isoforms and still retain efficacy.

Pro-fibrotic phenotypes (e.g., increased collagen deposit into the ECM) are associated not only with fibrosis, but also with aspects of cancer progression, such as invasion and metastasis. See, for example, Chakravarthy et al., (Nature Communications, (2018) 9:4692. “TGF-β-associated extracellular matrix genes link cancer-associated fibroblasts to immune evasion and immunotherapy failure”). Diseased tissues with dysregulated ECM, including stroma of various cancer types, can express both TGFβ1 and TGFβ3. Indeed, as recently as in 2019, multiple groups are making effort to develop TGFβ inhibitors that target both of these isoforms, such as ligand traps and integrin inhibitors.

Previously, we have shown that inhibition of TGFβ1 alone is sufficient to overcome primary resistant to checkpoint blockade therapy in tumor models. To further examine in vivo role of TGFβ3 in the regulation of ECM, a TGFβ31-selective inhibitor and TGFβ33-selective inhibitor were tested in a diet-induced murine liver fibrosis model.

In control animals that received regular diet, the baseline fibrosis score as measured by percentage of PSR-positive area by histology was less than 2%. After 12 weeks of fibrosis-causing diet (antibody treatment in the last 8 weeks, with continued diet), control animals treated with IgG alone showed approximately 6.5% of PSR-positive area by histology. Animals treated with the TGFβ31-selective inhibitor reduced that to approximately 4% of PSR-positive area (p<0.001 vs IgG control group). Animals treated with the TGFβ33-selective inhibitor were found to develop significantly worse fibrosis with approximately 12.5% PSR-positive area (p<0.001 vs IgG control group), while animals treated with a combination of the TGFβ33-selective inhibitor and the TGFβ31-selective inhibitor showed milder fibrosis with approximately 8% PSR-positive area (p<0.001 vs IgG control group).

These results suggest that inhibition of TGFβ3 exacerbated ECM dysregulation as indicated by increased collagen accumulation. Data also show that concurrent inhibition of the 113 isoforms in fact attenuates the efficacy of TGFβ1 inhibition in vivo, raising the possibility that TGFβ3 inhibition may be detrimental to ECM regulation.

### EQUIVALENTS

The various features and embodiments of the present disclosure, referred to in individual sections above apply, as appropriate, to other sections, mutatis mutandis. Consequently, features specified in one section may be combined with features specified in other sections, as appropriate.

Those skilled in the art will recognize, or be able to ascertain using no more than routine experimentation, many equivalents to the specific embodiments of the disclosure described herein. Such equivalents are intended to be encompassed by the following claims.

1.-21. (canceled)

22. A method for determining a circulating TGFβ level in a blood sample or sample derived therefrom, comprising:
(a) processing the blood sample or sample derived from blood at a temperature of 2-8° C. in a sample tube comprising an anticoagulant, wherein the anticoagulant comprises a citrate-theophylline-adenosine-dipyridamole (CTAD) solution; and
(b) carrying out one or more centrifugation steps; and/or carrying out a centrifugation protocol comprising a first centrifugation and a second centrifugation, and
(c) measuring a level of platelet factor 4 (PF4) in the blood sample or sample derived therefrom.

23. The method of 22, wherein the one or more centrifugation steps are carried out at a speed of greater than 100×g or below 15000×g.

24. The method of claim 22, wherein the first centrifugation of step (b) is slower than the second centrifugation.

25. The method of claim 22, wherein the first and second centrifugations of step (b) comprise:
(i) 10 minutes at 150×g and 20 minutes at 2500×g;
(ii) 10 minutes at 2500×g and 20 minutes at 2500×g; or
(iii) 10 minutes at 1500×g and 5 minutes at 12000×g.

26. The method of claim 22, wherein the anticoagulant comprises 0.11 M buffered trisodium citrate solution, 15 M theophylline, 3.7 M adenosine, and 0.198 M dipyridamole, and wherein the anticoagulant has a pH of about 5.0.

27. The method of claim 22, wherein the sample is used for determining a circulating TGFβ level only if the PF4 level in the sample is less than 500 ng/ml.

28. The method of claim 22, wherein the circulating TGFβ is circulating latent TGFβ1.

29. A method for treating cancer in a subject, comprising:
(i) determining a level of circulating TGFβ in a blood sample from the subject according to the method of claim 22;
(ii) administering to the subject a therapeutically effective dose of a TGFβ inhibitor;
(iii) determining a further level of circulating TGFβ in a blood sample from the subject; and
(iv) administering a further therapeutically effective dose of the TGFβ inhibitor if the level of circulating TGFβ in step (iii) is increased as compared to the level of circulating TGFβ in step (i);
wherein the increase is at least 1.5-fold.

30. The method of claim 29, further comprising administering a checkpoint inhibitor and/or a genotoxic therapy.

31. The method of claim 30, wherein the checkpoint inhibitor is an anti-PD-1 antibody, an anti-PD-L1 antibody, an anti-CTLA-4-antibody, an anti-LAG3 antibody, or an antigen-binding fragment thereof; and wherein the genotoxic therapy is a chemotherapy or a radiation therapy.

32. The method of claim 31, wherein the chemotherapy is a PARP inhibitor therapy.

33. The method of claim 29, wherein the subject has an advanced cancer and/or a solid cancer.

34. The method of claim 33, wherein the advanced cancer and/or the solid cancer is selected from melanoma, breast cancer, colorectal cancer, lung cancer, esophageal cancer, pancreatic cancer, bladder cancer, kidney cancer, uterine cancer, stomach cancer, head and neck cancer, urothelial carcinoma, hepatocellular carcinoma, thyroid cancer, and tenosynovial giant cell tumor (TGCT).

35. The method of claim 34, wherein the melanoma is metastatic melanoma; the renal cell carcinoma, the breast cancer is triple-negative breast cancer or HER2-positive breast cancer; the colorectal cancer is microsatellite stable-colorectal cancer or colon adenocarcinoma; the lung cancer is metastatic non-small cell lung cancer or small cell lung cancer; the kidney cancer is transitional cell carcinoma, renal sarcoma, or renal cell carcinoma (RCC); the uterine cancer is uterine corpus endometrial carcinoma or prostate cancer; the stomach cancer is gastric cancer; and the head and neck cancer is head and neck squamous cell cancer; and
wherein the RCC is clear cell RCC, papillary RCC, chromophobe RCC, collecting duct RCC, or unclassified RCC.

36. A method for treating a proliferative disorder in a subject, comprising:
(i) determining a level of P-Smad2 nuclear translocation in a biopsy sample from the subject;
(ii) administering to the subject a therapeutically effective dose of a TGFβ inhibitor;
(iii) further determining a level of the P-Smad2 nuclear translocation in a biopsy sample from the subject; and
(iv) administering a further therapeutically effective dose of the TGFβ inhibitor if the further determined level of P-Smad2 nuclear translocation of step (iii) is decreased compared to the level of P-Smad2 determined in step (i);
wherein the decrease is at least 1.3-fold.

37. The method of 36, wherein the level of P-Smad2 nuclear translocation is determined by immunohistochemistry and nuclear masking analysis.

38. A method for identifying an mMDSC population and a gMDSC population from a biological sample obtained from a subject comprising detection of cell surface markers comprising:
(i) applying a CD15+/CD66b^{high} filter; and
(ii) applying a CD15+/CD66b^{high}/CD14⁻/CD33^{low}/HLADR⁻/CD11 b⁺ filter;
wherein the mMDSC population is identified by the cell surface markers of CD11 b+, HLA−DR−/low, CD14+, CD15−, CD33+/high, and CD66b−; and wherein the gMDSC population is identified by the cell surface markers of CD11 b+, HLA−DR−, CD14−, CD15+, CD33+/low, and CD66b+.


SEQUENCE LISTING (180 sequences)

SEQ ID NO: 1 - protein, 8 aa, Artificial Sequence
  Description of Artificial Sequence: Synthetic peptide
GFTFSSFS

SEQ ID NO: 2 - protein, 8 aa, Artificial Sequence
  Description of Artificial Sequence: Synthetic peptide
ISPSADTI

SEQ ID NO: 3 - protein, 13 aa, Artificial Sequence
  Description of Artificial Sequence: Synthetic peptide
ARGVLDYGDMLMP

SEQ ID NO: 4 - protein, 6 aa, Artificial Sequence
  Description of Artificial Sequence: Synthetic peptide
QDITNY

SEQ ID NO: 5 - protein, 3 aa, Artificial Sequence
  Description of Artificial Sequence: Synthetic peptide
DAS

SEQ ID NO: 6 - protein, 10 aa, Artificial Sequence
  Description of Artificial Sequence: Synthetic peptide
QQADNHPPWT

SEQ ID NO: 7 - protein, 120 aa, Artificial Sequence
  Description of Artificial Sequence: Synthetic polypeptide
EVQLVESGGGLVQPGGSLRLSCTASGFTFSSFSMDWVRQAPGKGLEWVSYISPSADTIYYADSVKGRFTISRDNAKNTLYLQMNSLRAEDTAVYYCARGVLDYGDMLMPWGQGTLVTVSS

SEQ ID NO: 8 - protein, 108 aa, Artificial Sequence
  Description of Artificial Sequence: Synthetic polypeptide
DIQMTQSPSSLSASVGDRVTITCQASQDITNYLNWYQQKPGKAPKLLIYDASNLETGVPSRFSGSGSGTDFTFTISSLQPEDIATYYCQQADNHPPWTFGGGTKVEIK

SEQ ID NO: 9 - protein, 446 aa, Artificial Sequence
  Description of Artificial Sequence: Synthetic polypeptide
EVQLVESGGGLVQPGGSLRLSCTASGFTFSSFSMDWVRQAPGKGLEWVSYISPSADTIYYADSVKGRFTISRDNAKNTLYLQMNSLRAEDTAVYYCARGVLDYGDMLMPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 1338 aa, Artificial Sequence
  Description of Artificial Sequence: Synthetic polypeptide
GAGGTGCAGCTGGTGGAGTCTGGGGGAGGCTTGGTACAGCCTGGGGGGTCCCTGAGACTCTCCTGTACAGCCTCTGGATTCACCTTCAGTAGCTTCAGCATGGACTGGGTCCGCCAGGCTCCAGGGAAGGGGCTGGAGTGGGTTTCATACATTAGTCCCAGTGCAGACACCATATACTACGCAGACTCTGTGAAGGGCCGATTCACCATCTCCAGAGACAATGCCAAGAACACGCTGTATCTGCAAATGAACAGCCTGAGAGCCGAGGACACGGCGGTGTACTACTGCGCCAGAGGGGTGCTCGACTACGGAGACATGTTAATGCCATGGGGCCAGGGAACCCTGGTCACCGTCTCCTCAGCGTCGACCAAGGGCCCTTCCGTGTTCCCTCTGGCCCCTTGCTCCCGGTCCACCTCCGAGTCCACCGCCGCTCTGGGCTGTCTGGTGAAGGACTACTTCCCTGAGCCTGTGACCGTGAGCTGGAACTCTGGCGCCCTGACCTCCGGCGTGCACACCTTCCCTGCCGTGCTGCAGTCCTCCGGCCTGTACTCCCTGTCCTCCGTGGTGACCGTGCCTTCCTCCTCCCTGGGCACCAAGACCTACACCTGCAACGTGGACCACAAGCCTTCCAACACCAAGGTGGACAAGCGGGTGGAGTCCAAGTACGGCCCTCCTTGCCCTCCCTGCCCTGCCCCTGAGTTCCTGGGCGGACCCTCCGTGTTCCTGTTCCCTCCTAAGCCTAAGGACACCCTGATGATCTCCCGGACCCCTGAGGTGACCTGCGTGGTGGTGGACGTGTCCCAGGAAGATCCTGAGGTCCAGTTCAATTGGTACGTGGATGGCGTGGAGGTGCACAACGCCAAGACCAAGCCTCGGGAGGAACAGTTCAACTCCACCTACCGGGTGGTGTCTGTGCTGACCGTGCTGCACCAGGACTGGCTGAACGGCAAGGAATACAAGTGCAAGGTCAGCAACAAGGGCCTGCCCTCCTCCATCGAGAAAACCATCTCCAAGGCCAAGGGCCAGCCTCGCGAGCCTCAGGTGTACACCCTGCCTCCTAGCCAGGAAGAGATGACCAAGAATCAGGTGTCCCTGACATGCCTGGTGAAGGGCTTCTACCCTTCCGATATCGCCGTGGAGTGGGAGAGCAACGGCCAGCCAGAGAACAACTACAAGACCACCCCTCCTGTGCTGGACTCCGACGGCTCCTTCTTCCTGTACTCCAGGCTGACCGTGGACAAGTCCCGGTGGCAGGAAGGCAACGTCTTTTCCTGCTCCGTGATGCACGAGGCCCTGCACAACCACTACACCCAGAAGTCCCTGTCCCTGTCTCTGGGC

SEQ ID NO: 11 - protein, 215 aa, Artificial Sequence
  Description of Artificial Sequence: Synthetic polypeptide
DIQMTQSPSSLSASVGDRVTITCQASQDITNYLNWYQQKPGKAPKLLIYDASNLETGVPSRFSGSGSGTDFTFTISSLQPEDIATYYCQQADNHPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 645 aa, Artificial Sequence
  Description of Artificial Sequence: Synthetic polypeptide
GACATCCAGATGACCCAGTCTCCATCCTCCCTGTCTGCATCTGTAGGAGACAGAGTCACCATCACTTGCCAGGCGAGTCAGGACATTACCAACTATTTAAATTGGTATCAGCAGAAACCAGGGAAAGCCCCTAAGCTCCTGATCTACGATGCATCCAATTTGGAAACAGGGGTCCCATCAAGGTTCAGTGGAAGTGGATCTGGGACAGATTTTACTTTCACCATCAGCAGCCTGCAGCCTGAAGATATTGCAACATATTACTGTCAGCAGGCCGACAATCACCCTCCTTGGACTTTTGGCGGAGGGACCAAGGTTGAGATCAAACGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGT

SEQ ID NO: 13 - protein, 361 aa, Unknown
  Description of Unknown: TGFbeta1 sequence
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 14 - protein, 361 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 15 - protein, 360 aa, Artificial Sequence
  Description of Artificial Sequence: Synthetic polypeptide
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 16 - protein, 360 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 17 - protein, 395 aa, Unknown
  Description of Unknown: TGFbeta2 seqeunce
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 18 - protein, 395 aa, Artificial Sequence
  Description of Artificial Sequence: Synthetic polypeptide
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 19 - protein, 394 aa, Artificial Sequence
  Description of Artificial Sequence: Synthetic polypeptide
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 20 - protein, 394 aa, Artificial Sequence
  Description of Artificial Sequence: Synthetic polypeptide
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 21 - protein, 392 aa, Unknown
  Description of Unknown: TGFbeta3 sequence
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 22 - protein, 392 aa, Artificial Sequence
  Description of Artificial Sequence: Synthetic polypeptide
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 23 - protein, 391 aa, Artificial Sequence
  Description of Artificial Sequence: Synthetic polypeptide
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 24 - protein, 391 aa, Artificial Sequence
  Description of Artificial Sequence: Synthetic polypeptide
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 25 - protein, 361 aa, Mus sp.
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 26 - protein, 361 aa, Macaca fascicularis
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 27 - protein, 249 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRR

SEQ ID NO: 28 - protein, 249 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRR

SEQ ID NO: 29 - protein, 360 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHGALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 30 - protein, 361 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 31 - protein, 361 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 32 - protein, 360 aa, Artificial Sequence
  Description of Artificial Sequence: Synthetic polypeptide
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 33 - protein, 1260 aa, Macaca fascicularis
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRAGALSTGALPPLAPEGDSVASKHAIYAVQVIADPPGPGEGPPAQHAAFLVPLGPGQISAEVQAPPPVVNVRVHHPPEASVQVHRIESSNAEGAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPADGTAAFKEICPAGKGYHILTSHQTLTIQGESDFSLFLHPDGPPKPQQLPESPSQAPPPEDTEEERGVTTDSPVSEERSVQQSHPTATTSPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCVDVDECEAGDVCDNGICTNTPGSFQCQCLSGYHLSRDRSHCEDIDECDFPAACIGGDCINTNGSYRCLCPQGHRLVGGRKCQDIDECTQDPGLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGAEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPQAGPALTFDDCCCRQGRGWGAQCRPCPPRGAGSQCPTSQSESNSFWDTSPLLLGKPRRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 34 - protein, 1228 aa, Mus sp.
GPAGERGTGGGGALARERFKVVFAPVICKRTCLKGQCRDSCQQGSNMTLIGENGHSTDTLTGSAFRVVVCPLPCMNGGQCSSRNQCLCPPDFTGRFCQVPAAGTGAGTGSSGPGLARTGAMSTGPLPPLAPEGESVASKHAIYAVQVIADPPGPGEGPPAQHAAFLVPLGPGQISAEVQAPPPVVNVRVHHPPEASVQVHRIEGPNAEGPASSQHLLPHPKPPHPRPPTQKPLGRCFQDTLPKQPCGSNPLPGLTKQEDCCGSIGTAWGQSKCHKCPQLQYTGVQKPVPVRGEVGADCPQGYKRLNSTHCQDINECAMPGNVCHGDCLNNPGSYRCVCPPGHSLGPLAAQCIADKPEEKSLCFRLVSTEHQCQHPLTTRLTRQLCCCSVGKAWGARCQRCPADGTAAFKEICPGKGYHILTSHQTLTIQGESDFSLFLHPDGPPKPQQLPESPSRAPPLEDTEEERGVTMDPPVSEERSVQQSHPTTTTSPPRPYPELISRPSPPTFHRFLPDLPPSRSAVEIAPTQVTETDECRLNQNICGHGQCVPGPSDYSCHCNAGYRSHPQHRYCVDVNECEAEPCGPGKGICMNTGGSYNCHCNRGYRLHVGAGGRSCVDLNECAKPHLCGDGGFCINFPGHYKCNCYPGYRLKASRPPICEDIDECRDPSTCPDGKCENKPGSFKCIACQPGYRSQGGGACRDVNECSEGTPCSPGWCENLPGSYRCTCAQYEPAQDGLSCIDVDECEAGKVCQDGICTNTPGSFQCQCLSGYHLSRDRSRCEDIDECDFPAACIGGDCINTNGSYRCLCPLGHRLVGGRKCKKDIDECSQDPGLCLPHACENLQGSYVCVCDEGFTLTQDQHGCEEVEQPHHKKECYLNFDDTVFCDSVLATNVTQQECCCSLGAGWGDHCEIYPCPVYSSAEFHSLVPDGKRLHSGQQHCELCIPAHRDIDECILFGAEICKEGKCVNTQPGYECYCKQGFYYDGNLLECVDVDECLDESNCRNGVCENTRGGYRCACTPPAEYSPAQAQCLIPERWSTPQRDVKCAGASEERTACVWGPWAGPALTFDDCCCRQPRLGTQCRPCPPRGTGSQCPTSQSESNSFWDTSPLLLGKSPRDEDSSEEDSDECRCVSGRCVPRPGGAVCECPGGFQLDASRARCVDIDECRELNQRGLLCKSERCVNTSGSFRCVCKAGFTRSRPHGPACLSAAADDAAIAHTSVIDHRGYFH

SEQ ID NO: 35 - protein, 1373 aa, Macaca fascicularis
NHTGRIKVVFTPSICKVTCTKGSCQNSCEKGNTTTLISENGHAADTLTATNFRVVLCHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYKFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCTNGDCSNLEGSYMCSCHKGYTRTPDHKHCKDIDECQQGNLCVNGQCKNTEGSFRCTCGQGYQLSAAKDQCEDIDECQHHHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVEQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPMKDSDDYAQLCNIPVTGRRQPYGRDALVDFSEQYAPEADPYFIQDRFLNSFEELQAEECGILNGCENGRCVRVQEGYTCDCFDGYHLDTAKMTCVDVNECDELNNRMSLCKNAKCINTEGSYKCLCLPGYVPSDKPNYCTPLNTALNLEKDSDLE

SEQ ID NO: 36 - protein, 1374 aa, Mus sp.
NHTGRIKVVFTPSICKVTCTKGNCQNSCQKGNTTTLISENGHAADTLTATNFRVVICHLPCMNGGQCSSRDKCQCPPNFTGKLCQIPVLGASMPKLYQHAQQQGKALGSHVIHSTHTLPLTMTSQQGVKVKFPPNIVNIHVKHPPEASVQIHQVSRIDSPGGQKVKEAQPGQSQVSYQGLPVQKTQTVHSTYSHQQLIPHVYPVAAKTQLGRCFQETIGSQCGKALPGLSKQEDCCGTVGTSWGFNKCQKCPKKQSYHGYTQMMECLQGYKRVNNTFCQDINECQLQGVCPNGECLNTMGSYRCSCKMGFGPDPTFSSCVPDPPVISEEKGPCYRLVSPGRHCMHPLSVHLTKQICCCSVGKAWGPHCEKCPLPGTAAFKEICPGGMGYTVSGVHRRRPIHQHIGKEAVYVKPKNTQPVAKSTHPPPLPAKEEPVEALTSSWEHGPRGAEPEVVTAPPEKEIPSLDQEKTRLEPGQPQLSPGVSTIHLHPQFPVVVEKTSPPVPVEVAPEASTSSASQVIAPTQVTEINECTVNPDICGAGHCINLPVRYTCICYEGYKFSEQLRKCVDIDECAQVRHLCSQGRCENTEGSFLCVCPAGFMASEEGTNCIDVDECLRPDMCRDGRCINTAGAFRCEYCDSGYRMSRRGYCEDIDECLKPSTCPEEQCVNTPGSYQCVPCTEGFRGWNGQCLDVDECLQPKVCTNGSCTNLEGSYMCSCHRGYSPTPDHRHCQDIDECQQGNLCMNGQCRNTDGSFRCTCGQGYQLSAAKDQCEDIDECEHHHLCSHGQCRNTEGSFQCVCNQGYRASVLGDHCEDINECLEDSSVCQGGDCINTAGSYDCTCPDGFQLNDNKGCQDINECAQPGLCGSHGECLNTQGSFHCVCEQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRVTEDSGVDRQPREEKKECYYNLNDASLCDNVLAPNVTKQECCCTSGAGWGDNCEIFPCPVQGTAEFTEMCPRGKGLVPAGESSYDTGGENYKDADECLLFGEEICKNGYCLNTQPGYECYCKQGTYYDPVKLQCFDMDECQDPNSCIDGQCVNTEGSYNCFCTHPMVLDASEKRCVQPTESNEQIEETDVYQDLCWEHLSEEYVCSRPLVGKQTTYTECCCLYGEAWGMQCALCPMKDSDDYAQLCNIPVTGRRRPYGRDALVDFSEQYGPETDPYFIQDRFLNSFEELQAEECGILNGCENGRCVRVQEGYTCDCFDGYHLDMAKMTCVDVNECSELNNRMSLCKNAKCINTEGSYKCLCLPGYIPSDKPNYCTPLNSALNLDKESDLE

SEQ ID NO: 37 - protein, 646 aa, Mus sp.
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVNLILLLSFTLVSAIVLTTLATICFLRRQKLSQQYKA

SEQ ID NO: 38 - protein, 611 aa, Mus sp.
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVN

SEQ ID NO: 39 - protein, 1375 aa, Unknown
  Description of Unknown: LTBP1S sequence
NHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGVGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 40 - protein, 1260 aa, Unknown
  Description of Unknown: LTBP3 sequence
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRTGALSTGALPPLAPEGDSVASKHAIYAVQVIADPPGPGEGPPAQHAAFLVPLGPGQISAEVQAPPPVVNVRVHHPPEASVQVHRIESSNAESAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPTDGTAAFKEICPAGKGYHILTSHQTLTIQGESDFSLFLHPDGPPKPQQLPESPSQAPPPEDTEEERGVTTDSPVSEERSVQQSHPTATTTPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCLDVDECEAGDVCDNGICSNTPGSFQCQCLSGYHLSRDRSHCEDIDECDFPAACIGGDCINTNGSYRCLCPQGHRLVGGRKCQDIDECSQDPSLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGSEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPLAGPALTFDDCCCRQGRGWGAQCRPCPPRGAGSHCPTSQSESNSFWDTSPLLLGKPPRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 41 - protein, 645 aa, Unknown
  Description of Unknown: GARP sequence
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 42 - protein, 610 aa, Unknown
  Description of Unknown: sGARP sequence
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNIN

SEQ ID NO: 43 - protein, 5 aa, Artificial Sequence
  Description of Artificial Sequence: Synthetic peptide
CPPCP

SEQ ID NO: 44 - protein, 13 aa, Artificial Sequence
  Description of Artificial Sequence: Synthetic peptide
ASTKGPSVFPLAP

SEQ ID NO: 45 - protein, 6 aa, Artificial Sequence
  Description of Artificial Sequence: Synthetic peptide
ASTKGP

SEQ ID NO: 46 - protein, 12 aa, Artificial Sequence
  Description of Artificial Sequence: Synthetic peptide
TVAAPSVFIFPP

SEQ ID NO: 47 - protein, 5 aa, Artificial Sequence
  Description of Artificial Sequence: Synthetic peptide
TVAAP

SEQ ID NO: 48 - protein, 16 aa, Artificial Sequence
  Description of Artificial Sequence: Synthetic peptide
AKTTPKLEEGEFSEAR

SEQ ID NO: 49 - protein, 17 aa, Artificial Sequence
  Description of Artificial Sequence: Synthetic peptide
AKTTPKLEEGEFSEARV

SEQ ID NO: 50 - protein, 9 aa, Artificial Sequence
  Description of Artificial Sequence: Synthetic peptide
AKTTPKLGG

SEQ ID NO: 51 - protein, 10 aa, Artificial Sequence
  Description of Artificial Sequence: Synthetic peptide
SAKTTPKLGG

SEQ ID NO: 52 - protein, 6 aa, Artificial Sequence
  Description of Artificial Sequence: Synthetic peptide
SAKTTP

SEQ ID NO: 53 - protein, 6 aa, Artificial Sequence
  Description of Artificial Sequence: Synthetic peptide
RADAAP

SEQ ID NO: 54 - protein, 9 aa, Artificial Sequence
  Description of Artificial Sequence: Synthetic peptide
RADAAPTVS

SEQ ID NO: 55 - protein, 12 aa, Artificial Sequence
  Description of Artificial Sequence: Synthetic peptide
RADAAAAGGPGS

SEQ ID NO: 56 - protein, 27 aa, Artificial Sequence
  Description of Artificial Sequence: Synthetic peptide
RADAAAAGGGGSGGGGSGGGGSGGGGS

SEQ ID NO: 57 - protein, 18 aa, Artificial Sequence
  Description of Artificial Sequence: Synthetic peptide
SAKTTPKLEEGEFSEARV

SEQ ID NO: 58 - protein, 5 aa, Artificial Sequence
  Description of Artificial Sequence: Synthetic peptide
ADAAP

SEQ ID NO: 59 - protein, 12 aa, Artificial Sequence
  Description of Artificial Sequence: Synthetic peptide
ADAAPTVSIFPP

SEQ ID NO: 60 - protein, 6 aa, Artificial Sequence
  Description of Artificial Sequence: Synthetic peptide
QPKAAP

SEQ ID NO: 61 - protein, 13 aa, Artificial Sequence
  Description of Artificial Sequence: Synthetic peptide
QPKAAPSVTLFPP

SEQ ID NO: 62 - protein, 6 aa, Artificial Sequence
  Description of Artificial Sequence: Synthetic peptide
AKTTPP

SEQ ID NO: 63 - protein, 13 aa, Artificial Sequence
  Description of Artificial Sequence: Synthetic peptide
AKTTPPSVTPLAP

SEQ ID NO: 64 - protein, 6 aa, Artificial Sequence
  Description of Artificial Sequence: Synthetic peptide
AKTTAP

SEQ ID NO: 65 - protein, 13 aa, Artificial Sequence
  Description of Artificial Sequence: Synthetic peptide
AKTTAPSVYPLAP

SEQ ID NO: 66 - protein, 15 aa, Artificial Sequence
  Description of Artificial Sequence: Synthetic peptide
GGGGSGGGGSGGGGS

SEQ ID NO: 67 - protein, 15 aa, Artificial Sequence
  Description of Artificial Sequence: Synthetic peptide
GENKVEYAPALMALS

SEQ ID NO: 68 - protein, 15 aa, Artificial Sequence
  Description of Artificial Sequence: Synthetic peptide
GPAKELTPLKEAKVS

SEQ ID NO: 69 - protein, 15 aa, Artificial Sequence
  Description of Artificial Sequence: Synthetic peptide
GHEAAAVMQVQYPAS

SEQ ID NO: 70 - protein, 24 aa, Artificial Sequence
  Description of Artificial Sequence: Synthetic peptide
TVAAPSVFIFPPTVAAPSVFIFPP

SEQ ID NO: 71 - protein, 26 aa, Artificial Sequence
  Description of Artificial Sequence: Synthetic peptide
ASTKGPSVFPLAPASTKGPSVFPLAP

SEQ ID NO: 72 - protein, 692 aa, Homo sapiens
MELLPLWLCLGFHFLTVGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLYLVLILPSCLTLLVACTVIVLTFKKPLLQVIKSRCHWSSVY

SEQ ID NO: 73 - protein, 660 aa, Artificial Sequence
  Description of Artificial Sequence: Synthetic polypeptide
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLHHHHHH

SEQ ID NO: 74 - protein, 689 aa, Artificial Sequence
  Description of Artificial Sequence: Synthetic polypeptide
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLIIILTFILVSAILLTTLAACCCVRRQKFNQQYKA

SEQ ID NO: 75 - protein, 5 aa, Artificial Sequence
  Description of Artificial Sequence: Synthetic peptide
SFSMD

SEQ ID NO: 76 - protein, 17 aa, Artificial Sequence
  Description of Artificial Sequence: Synthetic peptide
YISPSADTIYYADSVKG

SEQ ID NO: 77 - protein, 11 aa, Artificial Sequence
  Description of Artificial Sequence: Synthetic peptide
GVLDYGDMLMP

SEQ ID NO: 78 - protein, 11 aa, Artificial Sequence
  Description of Artificial Sequence: Synthetic peptide
QASQDITNYLN

SEQ ID NO: 79 - protein, 7 aa, Artificial Sequence
  Description of Artificial Sequence: Synthetic peptide
DASNLET

SEQ ID NO: 80 - protein, 9 aa, Artificial Sequence
  Description of Artificial Sequence: Synthetic peptide
FTFSSFSMD

SEQ ID NO: 81 - protein, 9 aa, Artificial Sequence
  Description of Artificial Sequence: Synthetic peptide
FTFSSYSMN

SEQ ID NO: 82 - protein, 17 aa, Artificial Sequence
  Description of Artificial Sequence: Synthetic peptide
YISSSSSTIYYADSVKG

SEQ ID NO: 83 - protein, 13 aa, Artificial Sequence
  Description of Artificial Sequence: Synthetic peptide
ARGVLDYGDMLDP

SEQ ID NO: 84 - protein, 17 aa, Artificial Sequence
  Description of Artificial Sequence: Synthetic peptide
YISPDASTIYYADSVKG

SEQ ID NO: 85 - skipped

SEQ ID NO: 86 - protein, 17 aa, Artificial Sequence
  Description of Artificial Sequence: Synthetic peptide
YIHSDASTIYYADSVKG

SEQ ID NO: 87 - protein, 9 aa, Artificial Sequence
  Description of Artificial Sequence: Synthetic peptide
FTFSSFSMN

SEQ ID NO: 88 - protein, 9 aa, Artificial Sequence
  Description of Artificial Sequence: Synthetic peptide
FTFGSFSMN

SEQ ID NO: 89 - skipped

SEQ ID NO: 90 - protein, 9 aa, Artificial Sequence
  Description of Artificial Sequence: Synthetic peptide
FTFSFYAMN

SEQ ID NO: 91 - protein, 13 aa, Artificial Sequence
  Description of Artificial Sequence: Synthetic peptide
VRGVLDYGDMLDP

SEQ ID NO: 92 - protein, 9 aa, Artificial Sequence
  Description of Artificial Sequence: Synthetic peptide
FTFSSFAMN

SEQ ID NO: 93 - protein, 17 aa, Artificial Sequence
  Description of Artificial Sequence: Synthetic peptide
YISPDASTIYYAGSVKG

SEQ ID NO: 94 - protein, 13 aa, Artificial Sequence
  Description of Artificial Sequence: Synthetic peptide
VRAVLDYGDMLDP

SEQ ID NO: 95 - protein, 13 aa, Artificial Sequence
  Description of Artificial Sequence: Synthetic peptide
ARGTLDYGDMLDP

SEQ ID NO: 96 - protein, 13 aa, Artificial Sequence
  Description of Artificial Sequence: Synthetic peptide
ARAVLDYGDMLDP

SEQ ID NO: 97 - protein, 13 aa, Artificial Sequence
  Description of Artificial Sequence: Synthetic peptide
ARGVWDMGDMLDP

SEQ ID NO: 98 - protein, 13 aa, Artificial Sequence
  Description of Artificial Sequence: Synthetic peptide
AHGVLDYGDMLDP

SEQ ID NO: 99 - protein, 9 aa, Artificial Sequence
  Description of Artificial Sequence: Synthetic peptide
FTFAFYSMN

SEQ ID NO: 100 - protein, 120 aa, Artificial Sequence
  Description of Artificial Sequence: Synthetic polypeptide
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGVLDYGDMLDPWGQGTLVTVSS

SEQ ID NO: 101 - protein, 120 aa, Artificial Sequence
  Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 102 - protein, 120 aa, Artificial Sequence; identical to SEQ ID NO: 101
  Description of Artificial Sequence: Synthetic polypeptide

SEQ ID NO: 103 - protein, 120 aa, Artificial Sequence
  Description of Artificial Sequence: Synthetic polypeptide
EVQLVESGGGLVQPGGSLRLSCAASGFTFGSFSMNWVRQAPGKGLEWVSYIHSDASTIYYADSVKGRFTISRDNAKNSLYLQMNSLRAEDTAVYYCARGVLDYGDMLDPWGQGTLVTVSS

SEQ ID NO: 104 - protein, 120 aa, Artificial Sequence
  Description of Artificial Sequence: Synthetic polypeptide
EVQLVESGGGLVQPGGSLRLSCAASGFTFSSFSMNWVRQAPGKGLEWVSYISPSADTIYYADSVKGRFTISRDNAKNTLYLQMNSLRAEDTAVYYCARGVLDYGDMLDPWGQGTLVTVSS

SEQ ID NO: 105 - protein, 120 aa, Artificial Sequence
  Description of Artificial Sequence: Synthetic polypeptide
EVQLVESGGGLVQPGRSLRLSCAASGFTFSSFAMYWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 106 - protein, 120 aa, Artificial Sequence
  Description of Artificial Sequence: Synthetic polypeptide
EVQLVESGGGLVQPGGSLRLSCAASGFTFGSFSMDWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 107 - protein, 120 aa, Artificial Sequence
  Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNTLYLQMNSLRAEDTAVYYCARGVLDYGDMLDPWGQGTLVTVSS

SEQ ID NO: 108 - protein, 120 aa, Artificial Sequence
  Description of Artificial Sequence: Synthetic polypeptide
EVQLVESGGGLVQPGGSLRLSCAASGFTFSFYAMNWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 109 - protein, 120 aa, Artificial Sequence
  Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNSLYLQMNSLRAEDTAVYYCVRGVLDYGDMLDPWGQGTLVTVSS

SEQ ID NO: 110 - protein, 120 aa, Artificial Sequence
  Description of Artificial Sequence: Synthetic polypeptide
EVQLVESGGGLVQPGRSLRLSCAASGFTFSSFAMNWVRQAPGKGLEWVSYISPDASTIYYAGSVKGRFTISRDNAKNSLYLQMNSLRAEDTAVYYCVRAVLDYGDMLDPWGQGTLVTVSS

SEQ ID NO: 111 - protein, 120 aa, Artificial Sequence
  Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNSLYLQMNSLRAEDTAVYYCARGTLDYGDMLDPWGQGTLVTVSS

SEQ ID NO: 112 - protein, 120 aa, Artificial Sequence
  Description of Artificial Sequence: Synthetic polypeptide
EVQLVESGGGLVQPGGSLRLSCAASGFTFSSFSMDWVRQAPGKGLEWVSYISPDASTIYYADSVKGRFTISRDNAKNTLYLQMNSLRAEDTAVYYCARAVLDYGDMLDPWGQGTLVTVSS

SEQ ID NO: 113 - protein, 120 aa, Artificial Sequence
  Description of Artificial Sequence: Synthetic polypeptide
EVQLVESGGGLVQPGGSLRLSCAASGFTFSSFSMNWVRQAPGKGLEWVSYISPSADTIYYADSVKGRFTISRDNAKNTLYLQMNSLRAEDTAVYYCARGVWDMGDMLDPWGQGTLVTVSS

SEQ ID NO: 114 - protein, 120 aa, Artificial Sequence
  Description of Artificial Sequence: Synthetic polypeptide
EVQLVESGGGLVQPGGSLRLSCAASGFTFSSFSMNWVRQAPGKGLEWVSYISPSADTIYYADSVKGRFTISRDNAKNTLYLQMNSLRAEDTAVYYCAHGVLDYGDMLDPWGQGTLVTVSS

SEQ ID NO: 115 - protein, 120 aa, Artificial Sequence
  Description of Artificial Sequence: Synthetic polypeptide
EVQLVESGGGLVQPGGSLRLSCAASGFTFAFYSMNWVRQAPGKGLEWVSYISPDASTIYYADSVKGRFTISRDNAKNSLYLQMNSLRAEDTAVYYCARGVLDYGDMLDPWGQGTLVTVSS

SEQ ID NO: 116-118 - skipped (3 entries)

SEQ ID NO: 119 - protein, 249 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 120 - protein, 45 aa, Homo sapiens
LSTCKTIDMELVKRKRIEAIRGQILSKLRLASPPSQGEVPPGPLP

SEQ ID NO: 121 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 122 - protein, 4 aa, Homo sapiens
RHRR

SEQ ID NO: 123 - protein, 204 aa, Homo sapiens
EAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 124 - protein, 29 aa, Homo sapiens
CVRQLYIDFRKDLGWKWIHEPKGYHANFC

SEQ ID NO: 125 - protein, 35 aa, Homo sapiens
CVPQALEPLPIVYYVGRKPKVEQLSNMIVRSCKCS

SEQ ID NO: 126 - protein, 15 aa, Homo sapiens
LASPPSQGEVPPGPL

SEQ ID NO: 127 - protein, 27 aa, Homo sapiens
LASPPSQGEVPPGPLPEAVLALYNSTR

SEQ ID NO: 128 - protein, 29 aa, Homo sapiens
LSTCKTIDMELVKRKRIEAIRGQILSKLR

SEQ ID NO: 129 - protein, 10 aa, Homo sapiens
AVLALYNSTR

SEQ ID NO: 130 - protein, 20 aa, Homo sapiens
NGFTTGRRGDLATIHGMNRP

SEQ ID NO: 131 - protein, 13 aa, Homo sapiens
CSCDSRDNTLQVD

SEQ ID NO: 132 - protein, 9 aa, Homo sapiens
LVKRKRIEA

SEQ ID NO: 133 - protein, 11 aa, Homo sapiens
LASPPSQGEVP

SEQ ID NO: 134 - protein, 8 aa, Homo sapiens
PGPLPEAV

SEQ ID NO: 135 - protein, 8 aa, Homo sapiens
LALYNSTR

SEQ ID NO: 136 - protein, 9 aa, Homo sapiens
REAVPEPVL

SEQ ID NO: 137 - protein, 10 aa, Homo sapiens
YQKYSNNSWR

SEQ ID NO: 138 - protein, 19 aa, Homo sapiens
RKDLGWKWIHEPKGYHANF

SEQ ID NO: 139 - protein, 9 aa, Homo sapiens
LGPCPYIWS

SEQ ID NO: 140 - protein, 8 aa, Homo sapiens
ALEPLPIV

SEQ ID NO: 141 - protein, 10 aa, Homo sapiens
VGRKPKVEQL

SEQ ID NO: 142 - protein, 23 aa, Unknown
  Description of Unknown: proTGFbeta1 Region 1 sequence
KLRLASPPSQGEVPPGPLPEAVL

SEQ ID NO: 143 - protein, 8 aa, Unknown
  Description of Unknown: antibody binding region seqeuence
RKDLGWKW

SEQ ID NO: 144 - protein, 11 aa, Unknown
  Description of Unknown: antibody binding region seqeuence
RKDLGWKWIHE

SEQ ID NO: 145 - protein, 10 aa, Unknown
  Description of Unknown: antibody binding region seqeuence
HEPKGYHANF

SEQ ID NO: 146 - skipped

SEQ ID NO: 147 - protein, 26 aa, Artificial Sequence
  Description of Artificial Sequence: Synthetic peptide
EVQLVESGGGLVQPGGSLRLSCAASG

SEQ ID NO: 148 - protein, 14 aa, Artificial Sequence
  Description of Artificial Sequence: Synthetic peptide
WVRQAPGKGLEWVS

SEQ ID NO: 149 - protein, 30 aa, Artificial Sequence
  Description of Artificial Sequence: Synthetic polypeptide
RFTISRDNAKNSLYLQMNSLRAEDTAVYYC

SEQ ID NO: 150 - protein, 11 aa, Artificial Sequence
  Description of Artificial Sequence: Synthetic peptide
WGQGTLVTVSS

SEQ ID NO: 151 - protein, 23 aa, Artificial Sequence
  Description of Artificial Sequence: Synthetic peptide
DIQMTQSPSSLSASVGDRVTITC

SEQ ID NO: 152 - protein, 15 aa, Artificial Sequence
  Description of Artificial Sequence: Synthetic peptide
WYQQKPGKAPKLLIY

SEQ ID NO: 153 - protein, 32 aa, Artificial Sequence
  Description of Artificial Sequence: Synthetic polypeptide
GVPSRFSGSGSGTDFTFTISSLQPEDIATYYC

SEQ ID NO: 154 - protein, 10 aa, Artificial Sequence
  Description of Artificial Sequence: Synthetic peptide
FGGGTKVEIK

SEQ ID NO: 155-164 - skipped (10 entries)

SEQ ID NO: 165 - protein, 18 aa, Homo sapiens
SPPSQGEVPPGPLPEAVL

SEQ ID NO: 166 - protein, 14 aa, Homo sapiens
WKWIHEPKGYHANF

SEQ ID NO: 167 - protein, 9 aa, Homo sapiens
PGPLPEAVL

SEQ ID NO: 168 - protein, 7 aa, Artificial Sequence
  Description of Artificial Sequence: Synthetic peptide
GFTFSSF

SEQ ID NO: 169 - protein, 6 aa, Artificial Sequence
  Description of Artificial Sequence: Synthetic peptide
SPSADT

SEQ ID NO: 170 - protein, 14 aa, Homo sapiens
ASPPSQGEVPPGPL

SEQ ID NO: 171 - protein, 6 aa, Unknown
  Description of Unknown: TGFbeta amino acid sequence
LSKLRL

SEQ ID NO: 172 - protein, 367 aa, Artificial Sequence
  Description of Artificial Sequence: Synthetic polypeptide
HHHHHHLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 173 - protein, 22 aa, Unknown
  Description of Unknown: proTGFbeta2 Region 1 sequence
KLKLTSPPEDYPEPEEVPPEVI

SEQ ID NO: 174 - protein, 21 aa, Unknown
  Description of Unknown: proTGFbeta3 Region 1 sequence
KLRLTSPPEPTVMTHVPYQVL

SEQ ID NO: 175 - protein, 19 aa, Unknown
  Description of Unknown: proTGFbeta2 Region 2 sequence
KRDLGWKWIHEPKGYNANF

SEQ ID NO: 176 - protein, 19 aa, Unknown
  Description of Unknown: proTGFbeta3 Region 2 sequence
RQDLGWKWVHEPKGYYANF

SEQ ID NO: 177 - protein, 10 aa, Unknown
  Description of Unknown: proTGFbeta2 Region 3 sequence
IGKTPKIEQL

SEQ ID NO: 178 - protein, 10 aa, Unknown
  Description of Unknown: proTGFbeta3 Region 3 sequence
VGRTPKVEQL

SEQ ID NO: 179 - protein, 9 aa, Artificial Sequence
  Description of Artificial Sequence: Synthetic peptide
FTFSSFAMY

SEQ ID NO: 180 - protein, 9 aa, Artificial Sequence
  Description of Artificial Sequence: Synthetic peptide
FTFGSFSMD